(19) United States
(12) Patent Application Publication
(10) Pub. No.: US 2025/0051375 A1
(43) Pub. Date: Feb. 13, 2025

(54) ANTI-HIV COMPOUNDS

(71) Applicant: Gilead Sciences, Inc., Foster City, CA (US)

(72) Inventors: Gediminas J. Brizgys, San Carlos, CA (US); Michael O’ Neil Hanrahan Clarke, Redwood City, CA (US); Raheel Fondekar, Belmont, CA (US); Bindu Goyal, Fremont, CA (US); Luisruben P. Martinez, San Mateo, CA (US); Bradley Thomas Reid, Redwood City, CA (US); Nathan D. Shapiro, Belmont, CA (US); Doris T. Tang, Burlingame, CA (US)

(21) Appl. No.: 18/677,586

(22) Filed: May 29, 2024

Related U.S. Application Data
(60) Provisional application No. 63/470,139, filed on May 31, 2023.

(51) Int. Cl. C07F 9/6561; A61K 31/675

(52) U.S. Cl. CPC C07F 9/6561; A61K 31/675

(57) ABSTRACT

The invention provides compounds having Formula (I):

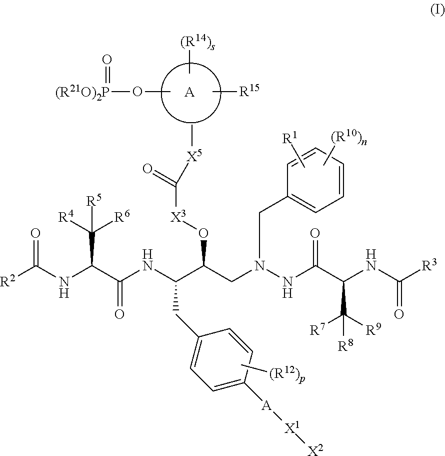

or a pharmaceutically acceptable salt thereof, as well as pharmaceutical compositions comprising the same, processes for their preparation, and methods of treating and preventing HIV infection by their administration.

### CROSS REFERENCE TO RELATED APPLICATIONS

This application claims priority to U.S. Provisional Patent Application No. 63/470,139 filed on May 31, 2023, which is hereby incorporated by reference in its entirety.

### FIELD

The present disclosure relates to compounds for use in the treatment of a Retroviridae viral infection including an infection caused by the HIV virus. The present disclosure also relates to intermediates for their preparation and to pharmaceutical compositions containing those compounds.

### BACKGROUND

Human immunodeficiency virus (HIV) infection and related diseases are a major public health problem worldwide. Human immunodeficiency virus type 1 (HIV-1) encodes three enzymes which are required for viral replication: reverse transcriptase, protease, and integrase. Several protease inhibitors (PI) are presently approved for use in AIDS or HIV. Others are in development.

Yet many protease inhibitors suffer from high rates of hepatic metabolism, which may require co-administration of a booster or more frequent dosing. Furthermore, viral resistance remains a problem. Accordingly, there is a need for new agents that inhibit the replication of HIV.

### SUMMARY

The present disclosure provides compounds and methods for the treatment of an HIV infection. Accordingly, the invention provides a compound of Formula (I):

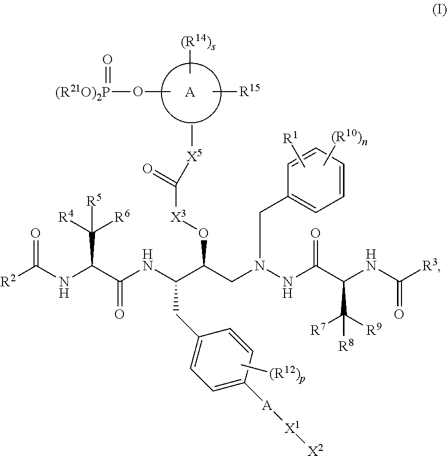

or a pharmaceutically acceptable salt thereof, wherein:
R¹ is a 5 to 10-membered heterocyclyl having 1 to 5 heteroatoms selected from N, O, and S, or a 5 to 10-membered heteroaryl having 1 to 5 heteroatoms selected from N, O, and S, wherein the 5 to 10-membered heterocyclyl or 5 to 10-membered heteroaryl is optionally substituted with 1 to 5 R^{a} groups;
R² and R³ are each independently C₁₋₄ alkyl, C₃₋₆ cycloalkyl, O—R^{2A}, C₁₋₂ alkyl-O—R^{2A}, N—(R^{3A})₂, or C₁₋₂ alkyl-N—(R^{3A})₂,
   wherein each R^{2A} is independently C₁₋₄ alkyl, C₃₋₆ cycloalkyl, or a 4 to 10-membered heterocyclyl having 1 to 5 heteroatoms selected from N, O, and S, wherein each R^{3A} is independently hydrogen, C₁₋₄ alkyl, C₃₋₆ cycloalkyl, or COO(R^{e}), wherein each R^{e} is independently hydrogen or C₁₋₄ alkyl, and wherein each C₃₋₆ cycloalkyl or 4 to 10-membered heterocyclyl is optionally substituted by 1 to 3 R^{f} groups, wherein each R^{f} is independently C₁₋₂ alkyl or halogen;
R⁴ is hydrogen, halo, C₁₋₄ alkyl, C₁₋₄ haloalkyl, C₃₋₆ cycloalkyl, C₁₋₄ alkoxy, or C₁₋₄ haloalkoxy;
R⁷ is hydrogen, halo, C₁₋₄ alkyl, C₁₋₄ haloalkyl, C₃₋₆ cycloalkyl, C₁₋₄ alkoxy, or C₁₋₄ haloalkoxy;
R⁵, R⁶, R⁸, and R⁹ are each independently hydrogen, halo, C₁₋₂ alkyl, C₁₋₂ haloalkyl, or C₃₋₆ cycloalkyl;
   and wherein two or more of R⁴, R⁵ and R⁶ or two or more of R⁷, R⁸, and R⁹ optionally join together to form one or more C₃₋₆ cycloalkyl groups that are optionally substituted with 1 to 4 groups selected from halogen, C₁₋₂ alkyl, and C₁₋₂haloalkyl;
each R¹⁰ is independently halogen, cyano, C₁₋₄ alkoxy, C₁₋₆ alkyl, or C₃₋₆ cycloalkyl;
n is 0 to 4;
each R^{a} is independently halogen, C₁₋₄ alkyl, C₁₋₄ alkyl with 1 to 2 groups selected from hydroxyl and C₁₋₄ alkoxy, C₁₋₄ haloalkyl, C₁₋₄ alkoxy, C₃₋₆ cycloalkyl, 4 to 10-membered heterocyclyl having 1 to 5 heteroatoms selected from N, O, and S which is optionally substituted with R^{a1}, or O—R^{3B},
   wherein R^{3B} is C₃₋₆ cycloalkyl optionally substituted with R^{a1} or a 4 to 10-membered heterocyclyl having 1 to 5 heteroatoms selected from N, O, and S optionally substituted with R^{a1},
   wherein each R^{a1} is independently C₁₋₄ alkyl, C₃₋₆ cycloalkyl, C₁₋₄ haloalkyl, or 4 to 8-membered heterocyclyl having 1 to 3 heteroatoms selected from N, O, and S;
A is ethynyl or a bond;
X¹ is a 6 to 10-membered aryl or a 5 to 10-membered heteroaryl having 1 to 3 heteroatoms selected from N, O, and S, wherein each 6 to 10-membered aryl or 5 to 10-membered heteroaryl is optionally substituted with 1 to 4 R^{b} groups;
X² is hydrogen or a 4 to 10-membered heterocyclyl having 1 to 5 heteroatoms selected from N, O, and S, wherein the 4 to 10-membered heterocyclyl is optionally substituted with one R¹¹ and optionally substituted with 1 to 5 R^{b} groups;
R¹¹ is —C═O(R^{e}), CH₂(R^{d}), S(O)₁₋₂(C₁₋₄ alkyl), S(O)₁₋₂—(C₃₋₆ cycloalkyl), a 4 to 10-membered heterocyclyl having 1 to 5 heteroatoms selected from N, O, and S, or a 5 to 9-membered heteroaryl having 1 to 5 heteroatoms selected from N, O, and S, wherein each 4 to 10-membered heterocyclyl or 5 to 9-membered heteroaryl is optionally substituted with 1 to 5 R^{b} groups;
   each R^{b} is independently halogen, oxo, C₁₋₄ alkyl, C₁₋₄ alkyl with 1 to 2 groups selected from hydroxyl and C₁₋₄ alkoxy, C₁₋₄ haloalkyl, C₁₋₄ alkoxy, or COO(R^{e});
   R^{e} is C₁₋₄ alkyl, C₁₋₄ haloalkyl, C₁₋₄ alkoxy, N(R^{e})₂, C₃₋₆ cycloalkyl, or a 4 to 6-membered heterocyclyl having 1 to 3 heteroatoms selected from N, O, and S, wherein the C₃₋₆ cycloalkyl and the 4 to 6-membered heterocyclyl are optionally substituted by 1 to 5 R^{b} groups;
   R^{d} is COO(R^{e}), N(R^{e})₂, C₃₋₆ cycloalkyl, or a 4 to 6-membered heterocyclyl having 1 to 3 heteroatoms selected from N, O, and S, wherein the C₃₋₆ cycloalkyl and the 4 to 6-membered heterocyclyl is optionally substituted by 1 to 5 R^{b} groups;
each R¹² is C₁₋₂ alkyl, halo, —OC₁₋₂ alkyl, or cyano;
each p is 0 to 4;
   X³ is —C(O)O—X⁴—O— or a bond;
   X⁴ is C₁₋₆ alkyl, C₂₋₆ alkenyl, or C₂₋₆ alkynyl, each optionally substituted with 1, 2, or 3 R^{x4};
   each R^{x4} is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   X⁵ is C₁₋₆ alkyl, C₂₋₆ alkenyl, or C₂₋₆ alkynyl, each optionally substituted with 1, 2, 3, or 4 R^{x5};
each R^{x5} is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   ring \textcircled{A} is 6- to 10-membered aryl or 5- to 10-membered heteroaryl;
   each R¹⁴ is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   s is 0, 1, 2, 3, or 4;
   R¹⁵ is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   each R²¹ is independently H or C₁₋₆ alkyl;
   each Z⁵ is independently C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³);
   each R²³ is independently hydrogen, C₁₋₆ alkyl, C₂₋₆ alkenyl, or C₂₋₆ alkynyl, wherein each C₁₋₆ alkyl, C₂₋₆ alkenyl, or C₂₋₆ alkynyl is optionally substituted with 1, 2, 3, or 4 Z⁶;
   each R²⁴ and R²⁵ is independently hydrogen, C₁₋₆ alkyl, C₂₋₆ alkenyl, C₂₋₆ alkynyl, C₃₋₆ cycloalkyl, 6- to 10-membered aryl, 5- to 10-membered heteroaryl, or 4 to 10-membered heterocyclyl, wherein each C₁₋₆ alkyl, C₂₋₆ alkenyl, C₂₋₆ alkynyl, C₃₋₆ cycloalkyl, 6- to 10-membered aryl, 5- to 10-membered heteroaryl and 4 to 10-membered heterocyclyl is optionally substituted with 1, 2, 3, or 4 Z⁶, or R²⁴ and R²¹, together with the nitrogen to which they are attached form a 4 to 10-membered heterocyclyl optionally substituted with 1, 2, 3, or 4 Z⁶;
   each Z⁶ is independently CN, C₁₋₆ alkyl, C₂₋₆ alkenyl, C₂₋₆ alkynyl, C₁₋₆ haloalkyl, 6- to 10-membered aryl, 5- to 10-membered heteroaryl, —OR²⁶, —C(O)R²⁶, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —OC(O)R²⁶, —OC(O)N(R²⁷)(R²⁸), P(O)(OR²⁶)₂, —OP(O)(OR²⁶)₂, —P(O)(R²⁶)(OR²⁶), —OP(O)(R²⁶)(OR²⁶), —SR²⁶, —S(O)R²⁶, —S(O)(NR²⁶)R²⁶, —S(O)₂R²⁶, S(O)₂OR²⁶, or —S(O)₂N(R²⁷)(R²⁸), or two Z6 can combine to form C₂₋₆ alkenyl, wherein each C₁₋₆ alkyl, C₂₋₆ alkenyl, C₂₋₆ alkynyl, 6- to 10-membered aryl, and 5- to 10-membered heteroaryl is optionally substituted with 1, 2, 3, or 4 Z⁷;
   each Z⁷ is independently CN, C₁₋₆ alkyl, C₁₋₆ haloalkyl, —OR²⁶, —C(O)R²⁶, —C(O)OR²⁶, —C(O)N(R²⁷)(R²⁸), —OC(O)R²⁶, —OC(O)N(R²⁷)(R²⁸), —S(O)₂N(R²⁷)(R²⁸), —P(O)(OR²⁶)₂, —OP(O)(OR²⁶)₂, —P(O)(R²⁶)(OR²⁶), or —OP(O)(R²⁶)(OR²⁶
   each R²⁶ is independently hydrogen, C₁₋₆ alkyl, C₂₋₆ alkenyl, or C₂₋₆ alkynyl; and
   each R²⁷ and R²⁸ is independently hydrogen, C₁₋₆ alkyl, C₂₋₆ alkenyl, or C₂₋₆ alkynyl.

Also provided is a pharmaceutical composition comprising a therapeutically effective amount of a compound disclosed herein, or a pharmaceutically acceptable salt thereof, and a pharmaceutically acceptable excipient. In certain embodiments, the pharmaceutical composition further comprises one, two, three, or four additional therapeutic agents selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, “antibody-like” therapeutic proteins, and combinations thereof.

Also provided is method of treating or preventing a Retroviridae viral infection (e.g., a human immunodeficiency virus (HIV) infection) comprising administering a therapeutically effective amount of a compound disclosed herein, or a pharmaceutically acceptable salt thereof, to a subject in need thereof. In some embodiments, provided herein is a method for treating or preventing an HIV infection in a patient, comprising administering to the mammal in need thereof a therapeutically effective amount of a compound of formula I, formula II, formula III, formula IV, formula V, formula VI, formula VII, formula VIII, formula IX, formula X, and/or formula XI, or a pharmaceutically acceptable salt thereof, in combination with a therapeutically effective amount of one, two, three, or four additional therapeutic agents selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, “antibody-like” therapeutic proteins, and combinations thereof. In some embodiments, provided herein is a method for treating or preventing an HIV infection in a heavily treatment-experienced patient, comprising administering to the mammal in need thereof a therapeutically effective amount of a compound of formula I, formula II, formula III, formula IV, formula V, formula VI, formula VII, formula VIII, formula IX, formula X, and/or formula XI, or a pharmaceutically acceptable salt thereof, in combination with a therapeutically effective amount of one, two, three, or four additional therapeutic agents selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, “antibody-like” therapeutic proteins, and combinations thereof.

Also provided is a compound disclosed herein, or a pharmaceutically acceptable salt thereof, for use in medical therapy (e.g., for use in treating or preventing a Retroviridae viral infection (e.g., an HIV viral infection) or the proliferation of the HIV virus or AIDS or delaying the onset of AIDS or AR^{c} symptoms in a mammal (e.g., a human)).

A compound disclosed herein, or a pharmaceutically acceptable salt thereof, for use in a method of treating or preventing a Retroviridae viral infection, a human immunodeficiency virus (HIV) infection or AIDS comprising administering a therapeutically effective amount of the compound to a patient in need thereof, is also provided. A compound disclosed herein, or a pharmaceutically acceptable salt thereof, for use in a method of treating or preventing a Retroviridae viral infection, a human immunodeficiency virus (HIV) infection or AIDS comprising administering a therapeutically effective amount of the compound to a heavily treatment-experienced patient in need thereof, is also provided.

Use of a compound disclosed herein, or a pharmaceutically acceptable salt thereof, in the manufacture of a medicament for treating or preventing a Retroviridae viral infection, a human immunodeficiency virus (HIV) infection or AIDS comprising administering a therapeutically effective amount of the compound to a patient in need thereof, is also provided. Use of a compound disclosed herein, or a pharmaceutically acceptable salt thereof, in the manufacture of a medicament for treating or preventing a Retroviridae viral infection, a human immunodeficiency virus (HIV) infection or AIDS comprising administering a therapeutically effective amount of the compound to a heavily treatment-experienced patient in need thereof, is also provided.

In certain embodiments, the current disclosure relates to an article of manufacture comprising a unit dosage of a compound disclosed herein, or a pharmaceutically acceptable salt thereof.

### DETAILED DESCRIPTION

The following is a list of abbreviations and acronyms used throughout the application:

### Abbreviation Meaning

° C. Degree Celsius
Ac Acetate
ACN Acetonitrile
AcOH Acetic acid
ATP Adenosine-5′-triphosphate
aq Aqueous
Bn Benzyl
CBz Benzyl chloroformate
d Doublet
DCE 1,2-dichloroethane
DCM Dichloromethane
dd Doublet of doublets
DIPEA N,N-diisopropylethylamine
DMA Dimethylacetamide
DME 1,2-dimethoxyethane
DMF Dimethylformamide
DMSO Dimethylsulfoxide
dppf 1,1′-Bis(diphenylphosphino)ferrocene
EDC 1-Ethyl-3-(3-dimethylaminopropyl)carbodiimide
EGTA Ethylene glycol tetraacetic acid
EtOAc Ethyl acetate
equiv/eq Equivalents
ESI Electrospray ionization
Et Ethyl
g Grams
h/hr Hours
HATU 2-(7-Aza-1H-Benzotriazole-1-yl)-1,1,3,3-tetramethyluronium hexafluorophosphate
hERG human Ether-á-go-go Related Gene
HPLC High-performance liquid chromatography
Hz Hertz
IC₅₀ The half maximal inhibitory concentration
J Coupling constant
Kg Kilogram
M Molar
m multiplet
m/z mass-to-charge ratio
M+ Mass peak
M+H Mass peak plus hydrogen
M−H Mass peak minus hydrogen
Me Methyl
MeOH Methyl alcohol/methanol
mg Milligram
MHz Megahertz
min/m Minute
mL/mL Milliliter
mM Millimolar
mmol Millimole
mol Mole
MNA 2-Methyl-4-nitroanilin
MS Mass spectroscopy
mw Microwave
N Normal
NMI N-methylimidazole
NMP N-methylpyrrolidinone
NMR Nuclear magnetic resonance
Ph Phenyl
PMB p-Methoxybenzyl
ppm Parts per million
prep Preparative
PSI Pound(s) per square inch
Rf Retention factor
RP Reverse phase
RT/rt Room temperature
s Second
s Singlet
STAB Sodium triacetoxyborohydride
t Triplet
TBAF Tetrabutylammonium fluoride
TBS tert-Butyldimethylsilyl
TCFH Chloro-N,N,N′,N′-tetramethylformamidinium
hexafluorophosphate
TEA Triethylamine
TEMPO (2,2,6,6-Tetramethylpiperidin-1-yl)oxyl or (2,2,6,6-tetramethylpiperidin-1-yl)oxidanyl
Tf Triflate
TFA Trifluoroacetic acid
THE Tetrahydrofuran
TLC Thin layer chromatography
TMS trimethylsilyl
WT Wild type
δ Chemical shift
μg Microgram
μL/μl Microliter
μM Micromolar
μm Micrometer
μmol Micromole

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

A dash at the front or end of a chemical group is a matter of convenience; chemical groups may be depicted with or without one or more dashes without losing their ordinary meaning. A wavy line drawn through a line in a structure indicates a point of attachment of a group, e.g.:

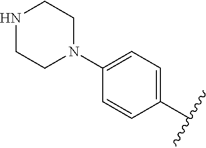 [SMILES: *c1ccc(N2CCNCC2)cc1]

A dashed line indicates an optional bond. Where multiple substituent groups are identified the point of attachment is at the terminal substituent (e.g., for “alkylaminocarbonyl” the point of attachment is at the carbonyl substituent).

The prefix “C_{x-y}” indicates that the following group has from x (e.g., 1) to y (e.g., 6) carbon atoms, one or more of which, in certain groups (e.g., heteroalkyl, heteroaryl, heteroarylalkyl, etc.), may be replaced with one or more heteroatoms or heteroatomic groups. For example, “C₁₋₆ alkyl” indicates that the alkyl group has from 1 to 6 carbon atoms. Likewise, the term “x-y membered” rings, wherein x and y are numerical ranges, such as “3 to 12-membered heterocyclyl”, refers to a ring containing x-y atoms (e.g., 3-12), of which up to 80% may be heteroatoms, such as N, O, S, P, and the remaining atoms are carbon.

Also, certain commonly used alternative chemical names may or may not be used. For example, a divalent group such as a divalent “alkyl” group, a divalent “aryl” group, etc., may also be referred to as an “alkylene” group or an “alkylenyl” group, or alkylyl group, an “arylene” group or an “arylenyl” group, or arylyl group, respectively.

“A compound disclosed herein” or “a compound of the present disclosure” refers to the compounds of Formula (I). Also included are the specific compounds of Examples 1-13.

“Alkyl” refers to any group derived from a linear or branched saturated hydrocarbon. Alkyl groups include, but are not limited to, methyl, ethyl, propyl such as propan-1-yl, propan-2-yl (iso-propyl), butyls such as butan-1-yl, butan-2-yl (sec-butyl), 2-methyl-propan-1-yl (iso-butyl), 2-methyl-propan-2-yl (t-butyl), pentyls, hexyls, octyls, dectyls, and the like. Unless otherwise specified, an alkyl group has from 1 to 10 carbon atoms, for example from 1 to 6 carbon atoms, for example from 1 to 4 carbon atoms.

“Alkenyl” refers to any group derived from a straight or branched hydrocarbon with at least one carbon-carbon double bond. Alkenyl groups include, but are not limited to, ethenyl (vinyl), propenyl (allyl), 1-butenyl, 1,3-butadienyl, and the like. Unless otherwise specified, an alkenyl group has from 2 to 10 carbon atoms, for example from 2 to 6 carbon atoms, for example from 2 to 4 carbon atoms.

“Alkynyl” refers to any group derived from a straight or branched hydrocarbon with at least one carbon-carbon triple bond and includes those groups having one triple bond and one double bond. Examples of alkynyl groups include, but are not limited to, ethynyl (—C≡C—), propargyl (—CH₂C≡C—), (E)-pent-3-en-1-ynyl, and the like. Unless otherwise specified, an alkynyl group has from 2 to 10 carbon atoms, for example from 2 to 6 carbon atoms, for example from 2 to 4 carbon atoms.

“Amino” refers to —NH₂. Amino groups may also be substituted as described herein, such as with alkyl, carbonyl or other amino groups. The term “alkylamino” refers to an amino group substituted with one or two alkyl substituents (e.g., dimethylamino or propylamino).

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1, 2, 3, 4-tetrahydronaphthyl. Aryl groups include, but are not limited to, those groups derived from acenaphthylene, anthracene, azulene, benzene, chrysene, a cyclopentadienyl anion, naphthalene, fluoranthene, fluorene, indane, perylene, phenalene, phenanthrene, pyrene and the like. Non-limiting examples of aryl groups include, but are not limited to, phenyl, indenyl, naphthyl, 1, 2, 3, 4-tetrahydronaphthyl, anthracenyl, and the like.

“Bridged” refers to a ring fusion wherein non-adjacent atoms on a ring are joined by a divalent substituent, such as an alkylenyl or heteroalkylenyl group or a single heteroatom. Quinuclidinyl and adamantanyl are examples of bridged ring systems.

The term “cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g., tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, spiro[3.3]heptane, and 1-cyclohex-3-enyl.

“Halo” and “halogen” refer to fluoro, chloro, bromo and iodo.

“Haloalkyl” refers to an alkyl wherein one or more hydrogen atoms are each replaced by a halogen. Examples include, but are not limited to, —CH₂Cl, —CH₂F, —CH₂Br, —CFClBr, —CH₂CH₂Cl, —CH₂CH₂F, —CF₃, —CH₂CF₃, —CH₂CCl₃, and the like, as well as alkyl groups such as perfluoroalkyl in which all hydrogen atoms are replaced by fluorine atoms.

“Alkoxy” or “alkoxyl” refers to a moiety of the formula —O-alkyl, wherein the alkyl portion is as defined above. For example, C₁₋₄ alkoxy refers to a moiety having 1-4 carbon alkyl group attached to the oxygen. “Haloalkoxy” or “haloalkoxyl” refers to a moiety of the formula —O-haloalkyl, wherein the haloalkyl portion is as defined above. For example, C₁₋₄ alkoxy refers to a moiety having 1-4 carbon halo alkyl group attached to the oxygen.

“Heteroalkyl” refers to an alkyl in which one or more of the carbon atoms (and any associated hydrogen atoms) are each independently replaced with the same or different heteroatom or heteroatomic group. Heteroatoms include, but are not limited to, N, P, O, S, etc. Heteroatomic groups include, but are not limited to, —NR—, —O—, —S—, —PH—, —P(O)₂—, —S(O)—, —S(O)₂—, and the like, where R is H, alkyl, aryl, cycloalkyl, heteroalkyl, heteroaryl or cycloheteroalkyl. Heteroalkyl groups include, but are not limited to, —OCH₃, —CH₂OCH₃, —SCH₃, —CH₂SCH₃, —NR^{c} H₃, —CH₂NR^{c} H₃, —CH₂OH and the like, where R is hydrogen, alkyl, aryl, arylalkyl, heteroalkyl, or heteroaryl, each of which may be optionally substituted. A heteroalkyl group comprises from 1 to 10 carbon and up to four three hetero atoms, e.g., from 1 to 6 carbon and from 1 to 2 hetero atoms.

“Heteroaryl” refers to mono or multicyclic aryl group in which one or more of the aromatic carbon atoms (and any associated hydrogen atoms) are independently replaced with the same or different heteroatom or heteroatomic group, as defined above. Multicyclic ring systems are included in heteroaryl and may be attached at the ring with the heteroatom or the aryl ring. Heteroaryl groups include, but are not limited to, groups derived from acridine, benzoimidazole, benzothiophene, benzofuran, benzoxazole, benzothiazole, carbazole, carboline, cinnoline, furan, imidazole, imidazopyridine, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyridone, pyrimidine, pyrrole, pyrrolizine, quinazoline, quinoline, quinolizine, quinoxaline, tetrazole, thiadiazole, thiazole, thiophene, triazole, xanthene, and the like. Heteroaryl groups may have 5-12 members, 5-10 members, or 5-6 members.

The term “heterocyclyl” or “heterocycle” as used herein refers to a single saturated or partially unsaturated non-aromatic ring or a non-aromatic multiple ring system that has at least one heteroatom in the ring (i.e., at least one annular heteroatom selected from oxygen, nitrogen, and sulfur). Unless otherwise specified, a heterocyclyl group has from 5 to about 20 annular atoms, for example from 3 to 12 annular atoms, for example from 3 to 10 annular atoms, for example from 5 to 10 annular atoms or for example from 5 to 6 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g., bicyclic heterocyclyl) system can be connected to each other via fused, spiro and bridged bonds when allowed by valency requirements. Heterocycles include, but are not limited to, groups derived from azetidine, aziridine, imidazolidine, morpholine, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, tetrahydro-2H-thiopyran 1,1-dioxide, quinuclidine, N-bromopyrrolidine, N-chloropiperidine, and the like. Heterocycles include spirocycles, such as, for example, aza or oxo-spiroheptanes. Heterocyclyl groups also include partially unsaturated ring systems containing one or more double bonds, including fused ring systems with one aromatic ring and one non-aromatic ring, but not fully aromatic ring systems. Examples include dihydroquinolines, e.g., 3,4-dihydroquinoline, dihydroisoquinolines, e.g., 1,2-dihydroisoquinoline, dihydroimidazole, tetrahydroimidazole, etc., indoline, isoindoline, isoindolones (e.g., isoindolin-1-one), isatin, dihydrophthalazine, quinolinone, spiro[cyclopropane-1,1′-isoindolin]-3′-one, and the like. Additional examples of heterocycles include 3,8-diazabicyclo[3.2.1]octanyl, 2,5-diazabicyclo[2.2.1]heptanyl, 3,6-diazabicyclo[3.1.1]heptanyl, 3-oxa-7,9-diazabicyclo[3.3.1]nonanyl, and hexahydropyrazino[2,1-c][1,4]oxazinyl, for example.

“Hydroxyl” and “hydroxy” are used interchangeably and refer to —OH. “Oxo” refers to

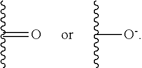

Where tautomeric forms of the compound exist, hydroxyl and oxo groups are interchangeable.

It is understood that combinations of chemical groups may be used and will be recognized by persons of ordinary skill in the art. For instance, the group “hydroxyalkyl” would refer to a hydroxyl group attached to an alkyl group. A great number of such combinations may be readily envisaged. Additional examples of substituent combinations used herein include: C₁₋₆ alkylaminocarbonyl (e.g., CH₃CH₂NHC(O)—) C₁₋₆ alkoxycarbonyl (e.g., CH₃O—C(O)—), 5-7 membered heterocyclyl-C₁₋₆ alkyl (e.g., piperazinyl-CH₂—), C₁₋₆ alkylsulfonyl-5-7 membered heterocyclyl (e.g., CH₃S(O)₂-morpholinyl-), 5-7 membered heterocyclyl C₁₋₆ alkoxy 5-7 membered heterocyclyloxy, (4-7 membered heterocyclyl)-4-7 membered heterocyclyl (e.g., oxetanyl-pyrrolidinyl-), C₃₋₆ cycloalkylaminocarbonyl (e.g., cyclopropyl-NH—C(O)—), 5-7 membered heterocyclyl-C₂₋₆ alkynyl (e.g., N-piperazinyl-CH₂C≡CCH₂_), and C₆₋₁₀ arylaminocarbonyl (e.g., phenyl-NH—C(O)—).

“Spiro” refers to a ring substituent which is joined by two bonds at the same carbon atom. Examples of spiro groups include 1,1-diethylcyclopentane, dimethyl-dioxolane, and 4-benzyl-4-methylpiperidine, wherein the cyclopentane and piperidine, respectively, are the spiro substituents. When substituents (R-groups) join together (e.g., when R⁷ and R⁸ join together) they may be taken from the same point of attachment to form a spiro ring.

The phrase “meta (3) position with respect to the point of attachment of the A ring”, refers to the position on the ring where the substituent (e.g., —CN) is adjoined and is shown below with an arrow, wherein z represents a carbon atom or nitrogen:

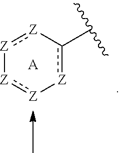

Similarly, para (4) position substitution refers to attachment of a substituent at the position indicated below, with respect to the point of attachment (e.g., of the B ring):

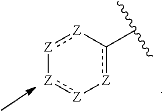

Similarly, ortho or 2-position refers to attachment of a substituent at the position indicated below, with respect to the point of attachment:

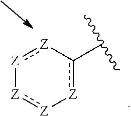

The compounds described herein include isomers, stereoisomers and the like. As used herein, the term “isomers” refers to different compounds that have the same molecular formula but differ in arrangement and configuration of the atoms. Also as used herein, the term “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound.

The term “fused” refers to a ring which is bound to an adjacent ring.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included. To the extent that compounds depicted herein are represented as having a particular stereochemistry, it is understood by one of skill in the art that such compounds may contain some detectable or undetectable levels of compounds sharing the same structure, but having different stereochemistry.

“IC₉₅” or “EC₉₅” refers to the inhibitory concentration required to achieve 95% of the maximum desired effect, which in many cases here is the inhibition of the HIV virus. This term is obtained using an in vitro assay evaluating the concentration-dependent inhibition of wild type HIV virus.

“IC₅₀” or “BC₅₀” refers to the inhibitory concentration required to achieve 50% of the maximum desired effect, which in many cases here is the inhibition of the HIV virus. This term is obtained using an in vitro assay evaluating the concentration-dependent inhibition of wild type HIV virus.

“IQ” or “inhibitory quotient” refers to the ratio between the trough drug concentration (Cₜₐᵤ) and level of drug resistance of the HIV isolate as determined by the IC₉₅ (i.e. Cₜₐᵤ/IC₉₅).

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, PA, (2005), at p. 732, Table 38-5, both of which are hereby incorporated by reference herein.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, pocket pets, rabbits, dogs, and monkeys), and the like.

As used herein, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and/or c) relieving the disease or condition, e.g., causing the regression of clinical symptoms, ameliorating the disease state, delaying the progression of the disease, increasing the quality of life, and/or prolonging survival.

As used herein, “delaying” development of a disease or condition means to defer, hinder, slow, retard, stabilize and/or postpone development of the disease or condition. This delay can be of varying lengths of time, depending on the history of the disease and/or subject being treated. As is evident to one skilled in the art, a sufficient or significant delay can, in effect, encompass prevention, in that the subject does not develop the disease or condition. For example, a method that “delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming a subject's HIV⁺ status and assessing the subject's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in subjects with functioning immune systems but does occur in AIDS patients). Development may also refer to disease progression that may be initially undetectable and includes occurrence, recurrence and onset.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable infectious agent (e.g., virus) in the subject). The subject may be an individual at risk of developing the disease or disorder, such as an individual who has one or more risk factors known to be associated with development or onset of the disease or disorder. Thus, the term “preventing HIV infection” refers to administering to a subject who does not have a detectable HIV infection an anti-HIV therapeutic substance. It is understood that the subject for anti-HIV preventative therapy may be an individual at risk of contracting the HIV virus. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease or to an amount that is effective to protect against the contracting or onset of a disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment outcome. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

The compounds of the invention include solvates, hydrates, tautomers, stereoisomers and salt forms thereof.

Provided are also compounds in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds exhibit may increase resistance to metabolism, and thus may be useful for increasing the half-life of the compounds when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

As referenced herein, darunavir is a HIV protease inhibitor having the structure:

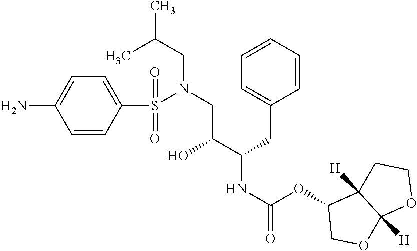 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

and having the IUPAC name [(3aS,4R,6aR)-2,3,3a,4,5,6a-hexahydrofuro[2,3-b]furan-4-yl] N-[(2S,3R)-4-[(4-aminophenyl)sulfonyl-(2-methylpropyl)amino]-3-hydroxy-1-phenylbutan-2-yl]carbamate. Darunavir (DRV) is marketed under the brand name PREZISTA®.

As referenced herein, atazanavir is a HIV protease inhibitor having the structure:

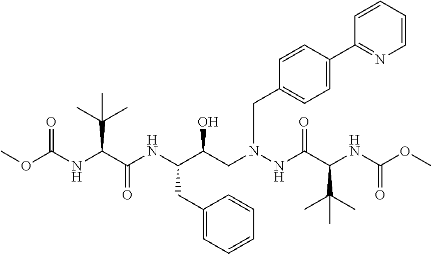

and having the IUPAC name methyl N-[(2S)-1-[2-[(2S,3S)-2-hydroxy-3-[[(2S)-2-(methoxycarbonylamino)-3,3-dimethylbutanoyl]amino]-4-phenylbutyl]-2-[(4-pyridin-2-ylphenyl)methyl]hydrazinyl]-3,3-dimethyl-1-oxobutan-2-yl]carbamate. Atazanavir (ATV) is marked under the brand name REYATAZ®.

### Compounds

The compounds disclosed herein can be used to treat or prevent, for example, HIV infection. In some embodiments, the compounds of the invention are prodrugs, which upon administration to the human body are converted to compounds having biological activity. The compounds disclosed herein may be metabolized in vivo to form one or more of the therapeutic compounds described in International Publication No. WO 2018/145021.

In certain embodiments, the compound is a compound of Formula (I):

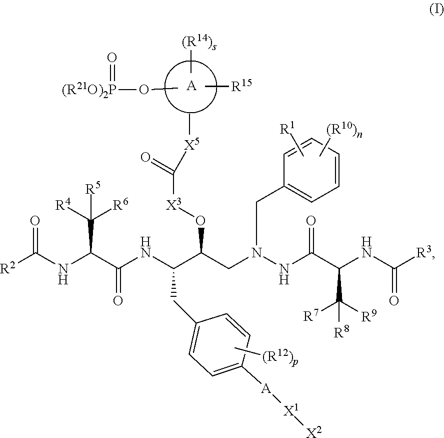

or a pharmaceutically acceptable salt thereof, wherein:
R¹ is a 5 to 10-membered heterocyclyl having 1 to 5 heteroatoms selected from N, O, and S, or a 5 to 10-membered heteroaryl having 1 to 5 heteroatoms selected from N, O, and S, wherein the 5 to 10-membered heterocyclyl or 5 to 10-membered heteroaryl is optionally substituted with 1 to 5 R^{a} groups;
R² and R³ are each independently C₁₋₄ alkyl, C₃₋₆ cycloalkyl, O—R^{2A}, C₁₋₂ alkyl-O—R^{2A}, N—(R^{3A})₂, or C₁₋₂ alkyl-N—(R^{3A})₂,
   wherein each R^{2A} is independently C₁₋₄ alkyl, C₃₋₆ cycloalkyl, or a 4 to 10-membered heterocyclyl having 1 to 5 heteroatoms selected from N, O, and S, wherein each R^{3A} is independently hydrogen, C₁₋₄ alkyl, C₃₋₆ cycloalkyl, or COO(R^{e}), wherein each R^{e} is independently hydrogen or C₁₋₄ alkyl,
   and wherein each C₃₋₆ cycloalkyl or 4 to 10-membered heterocyclyl is optionally substituted by 1 to 3 R^{f} groups, wherein each R^{f} is independently C₁₋₂ alkyl or halogen;
R⁴ is hydrogen, halo, C₁₋₄ alkyl, C₁₋₄ haloalkyl, C₃₋₆ cycloalkyl, C₁₋₄ alkoxy, or C₁₋₄ haloalkoxy;
R⁷ is hydrogen, halo, C₁₋₄ alkyl, C₁₋₄ haloalkyl, C₃₋₆ cycloalkyl, C₁₋₄ alkoxy, or C₁₋₄ haloalkoxy;
R⁵, R⁶, R⁸, and R⁹ are each independently hydrogen, halo, C₁₋₂ alkyl, C₁₋₂ haloalkyl, or C₃₋₆ cycloalkyl;
   and wherein two or more of R⁴, R⁵ and R⁶ or two or more of R⁷, R⁸, and R⁹ optionally join together to form one or more C₃₋₆ cycloalkyl groups that are optionally substituted with 1 to 4 groups selected from halogen, C₁₋₂ alkyl, and C₁₋₂haloalkyl;
each R¹⁰ is independently halogen, cyano, C₁₋₄ alkoxy, C₁₋₆ alkyl, or C₃₋₆ cycloalkyl;
n is 0 to 4;
each R^{a} is independently halogen, C₁₋₄ alkyl, C₁₋₄ alkyl with 1 to 2 groups selected from hydroxyl and C₁₋₄ alkoxy, C₁₋₄ haloalkyl, C₁₋₄ alkoxy, C₃₋₆ cycloalkyl, 4 to 10-membered heterocyclyl having 1 to 5 heteroatoms selected from N, O, and S which is optionally substituted with R^{a1}, or O—R^{3B},
   wherein R^{3B} is C₃₋₆ cycloalkyl optionally substituted with R^{a1} or a 4 to 10-membered heterocyclyl having 1 to 5 heteroatoms selected from N, O, and S optionally substituted with R^{a1},
   wherein each R^{a1} is independently C₁₋₄ alkyl, C₃₋₆ cycloalkyl, C₁₋₄ haloalkyl, or 4 to 8-membered heterocyclyl having 1 to 3 heteroatoms selected from N, O, and S;
A is ethynyl or a bond;
X¹ is a 6 to 10-membered aryl or a 5 to 10-membered heteroaryl having 1 to 3 heteroatoms selected from N, O, and S, wherein each 6 to 10-membered aryl or 5 to 10-membered heteroaryl is optionally substituted with 1 to 4 R^{b} groups;
X² is hydrogen or a 4 to 10-membered heterocyclyl having 1 to 5 heteroatoms selected from N, O, and S, wherein the 4 to 10-membered heterocyclyl is optionally substituted with one R¹¹ and optionally substituted with 1 to 5 R^{b} groups;
R¹¹ is —C═O(R^{c}), CH₂(R^{d}), S(O)₁₋₂(C₁₋₄ alkyl), S(O)₁₋₂—(C₃₋₆ cycloalkyl), a 4 to 10-membered heterocyclyl having 1 to 5 heteroatoms selected from N, O, and S, or a 5 to 9-membered heteroaryl having 1 to 5 heteroatoms selected from N, O, and S, wherein each 4 to 10-membered heterocyclyl or 5 to 9-membered heteroaryl is optionally substituted with 1 to 5 R^{b} groups;
   each R^{b} is independently halogen, oxo, C₁₋₄ alkyl, C₁₋₄ alkyl with 1 to 2 groups selected from hydroxyl and C₁₋₄ alkoxy, C₁₋₄ haloalkyl, C₁₋₄ alkoxy, or COO(R^{e});
   R^{c} is C₁₋₄ alkyl, C₁₋₄ haloalkyl, C₁₋₄ alkoxy, N(R^{e})₂, C₃₋₆ cycloalkyl, or a 4 to 6-membered heterocyclyl having 1 to 3 heteroatoms selected from N, O, and S, wherein the C₃₋₆ cycloalkyl and the 4 to 6-membered heterocyclyl are optionally substituted by 1 to 5 R^{b} groups;
   R^{d} is COO(R^{e}), N(R^{e})₂, C₃₋₆ cycloalkyl, or a 4 to 6-membered heterocyclyl having 1 to 3 heteroatoms selected from N, O, and S, wherein the C₃₋₆ cycloalkyl and the 4 to 6-membered heterocyclyl is optionally substituted by 1 to 5 R^{b} groups;
each R¹² is C₁₋₂ alkyl, halo, —OC₁₋₂ alkyl, or cyano;
each p is 0 to 4;
   X³ is —C(O)O—X⁴—O— or a bond;
   X⁴ is C₁₋₆ alkyl, C₂₋₆ alkenyl, or C₂₋₆ alkynyl, each optionally substituted with 1, 2, or 3 R^{x4};
   each R^{x4} is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   X⁵ is C₁₋₆ alkyl, C₂₋₆ alkenyl, or C₂₋₆ alkynyl, each optionally substituted with 1, 2, 3, or 4 R^{x5};
   each R^{x5} is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   ring \textcircled{A} is 6- to 10-membered aryl or 5- to 10-membered heteroaryl;
   each R¹⁴ is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   s is 0, 1, 2, 3, or 4;
   R¹⁵ is independently C₁₋₆ alkyl, C₂₋₆ alkenyl, C₂₋₆ alkynyl, C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³), wherein each C₁₋₆ alkyl, C₂₋₆ alkenyl, and C₂₋₆ alkynyl is optionally substituted with 1, 2, 3, or 4 Z⁵;
   each R²¹ is independently H or C₁₋₆ alkyl;
   each Z⁵ is independently C₁₋₆ haloalkyl, —OR²³, —C(O)R²³, —C(O)OR²³, —C(O)N(R²⁴)(R²⁵), —OC(O)R²³, —OC(O)N(R²⁴)(R²⁵), —P(O)(OR²³)₂, —OP(O)(OR²³)₂, —P(O)(R²³)(OR²³), or —OP(O)(R²³)(OR²³);
   each R²³ is independently hydrogen, C₁₋₆ alkyl, C₂₋₆ alkenyl, or C₂₋₆ alkynyl, wherein each C₁₋₆ alkyl, C₂₋₆ alkenyl, or C₂₋₆ alkynyl is optionally substituted with 1, 2, 3, or 4 Z⁶;
   each R²⁴ and R²⁵ is independently hydrogen, C₁₋₆ alkyl, C₂₋₆ alkenyl, C₂₋₆ alkynyl, C₃₋₆ cycloalkyl, 6- to 10-membered aryl, 5- to 10-membered heteroaryl, or 4 to 10-membered heterocyclyl, wherein each C₁₋₆ alkyl, C₂₋₆ alkenyl, C₂₋₆ alkynyl, C₃₋₆ cycloalkyl, 6- to 10-membered aryl, 5- to 10-membered heteroaryl and 4 to 10-membered heterocyclyl is optionally substituted with 1, 2, 3, or 4 Z⁶, or R²⁴ and R²⁵, together with the nitrogen to which they are attached form a 4 to 10-membered heterocyclyl optionally substituted with 1, 2, 3, or 4 Z⁶;
   each Z⁶ is independently CN, C₁₋₆ alkyl, C₂₋₆ alkenyl, C₂₋₆ alkynyl, C₁₋₆ haloalkyl, 6- to 10-membered aryl, 5- to 10-membered heteroaryl, —OR²⁶, —C(O)R²⁶, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —OC(O)R²⁶, —OC(O)N(R²⁷)(R²⁸), P(O)(OR²⁶)₂, —OP(O)(OR²⁶)₂, —P(O)(R²⁶)(OR²⁶), —OP(O)(R²⁶)(OR²⁶), —SR²⁶, —S(O)R²⁶, —S(O)(NR²⁶)R²⁶, —S(O)₂R²⁶, S(O)₂OR²⁶, or —S(O)₂N(R²⁷)(R²⁸), or two Z6 can combine to form C₂₋₆ alkenyl, wherein each C₁₋₆ alkyl, C₂₋₆ alkenyl, C₂₋₆ alkynyl, 6- to 10-membered aryl, and 5- to 10-membered heteroaryl is optionally substituted with 1, 2, 3, or 4 Z⁷;
   each Z⁷ is independently CN, C₁₋₆ alkyl, C₁₋₆ haloalkyl, —OR²⁶, —C(O)R²⁶, —C(O)OR²⁶, —C(O)N(R²⁷)(R²⁸), —OC(O)R²⁶, —OC(O)N(R²⁷)(R²⁸), —S(O)₂N(R²⁷)(R²⁸), —P(O)(OR²⁶)₂, —OP(O)(OR²⁶)₂, —P(O)(R²⁶)(OR²⁶), or —OP(O)(R²⁶)(OR²⁶
   each R²⁶ is independently hydrogen, C₁₋₆ alkyl, C₂₋₆ alkenyl, or C₂₋₆ alkynyl; and
   each R²⁷ and R²⁸ is independently hydrogen, C₁₋₆ alkyl, C₂₋₆ alkenyl, or C₂₋₆ alkynyl.

In certain embodiments:
X³ is a bond;
X⁵ is C₁₋₆ alkyl each optionally substituted with 1, 2, 3, or 4 R^{x5};
each R^{x5} is independently C₁₋₆ alkyl, C₂₋₆ alkenyl, C₁₋₆ haloalkyl, or —OR²³, wherein each C₁₋₆ alkyl and C₂₋₆ alkenyl is optionally substituted with 1, 2, 3, or 4 Z⁵;
ring \textcircled{A} is 6-membered aryl or 5- to 6-membered heteroaryl;
each R¹⁴ is independently C₁₋₆ alkyl, C₂₋₆ alkenyl, C₁₋₆ haloalkyl, —OR²³, —C(O)R²³, —C(O)OR²³, or —C(O)N(R²⁴)(R²⁵), wherein each C₁₋₆ alkyl and C₂₋₆ alkenyl is optionally substituted with 1, 2, 3, or 4 Z⁵;
s is 0, 1, 2, or 3;
R¹⁵ is independently C₁₋₆ alkyl, C₂₋₆ alkenyl, C₁₋₆ haloalkyl, —C(O)R²³, or —C(O)N(R²⁴)(R²⁵), wherein each C₁₋₆ alkyl and C₂₋₆ alkenyl is optionally substituted with 1, 2, 3, or 4 Z⁵;
each R²¹ is independently H or C₁₋₆ alkyl;
each Z⁵ is independently —C(O)OR²³ or —C(O)N(R²⁴)(R²⁵);
each R²³ is independently hydrogen or C₁₋₆ alkyl, wherein each C₁₋₆ alkyl is optionally substituted with 1, 2, 3, or 4 Z⁶;
each R²⁴ and R²⁵ is independently hydrogen, C₁₋₆ alkyl, 6- to 10-membered aryl, or 5- to 10-membered heteroaryl, wherein each C₁₋₆ alkyl, 6- to 10-membered aryl, or 5- to 10-membered heteroaryl is optionally substituted with 1, 2, 3, or 4 Z⁶, or R²⁴ and R²⁵, together with the nitrogen to which they are attached form a 4 to 10-membered heterocyclyl optionally substituted with 1, 2, 3, or 4 Z⁶;
each Z⁶ is independently C₁₋₆ alkyl, —C(O)R²⁶, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —P(O)(OR²⁶)₂, —OP(O)(OR²⁶)₂, —P(O)(R²⁶)(OR²⁶), —OP(O)(R²⁶)(OR²⁶), —S(O)₂R²⁶, S(O)₂OR²⁶, or —S(O)₂N(R²⁷)(R²⁸), wherein each C₁₋₆ alkyl is optionally substituted with 1, 2, 3, or 4 Z⁷;
each Z⁷ is independently —C(O)R²⁶, —C(O)OR²⁶, or —C(O)N(R²⁷)(R²⁸);
each R²⁶ is independently hydrogen, C₁₋₆ alkyl, C₂₋₆ alkenyl, or C₂₋₆ alkynyl; and
each R²⁷ and R²⁸ is independently hydrogen, C₁₋₆ alkyl, C₂₋₆ alkenyl, or C₂₋₆ alkynyl.

In certain embodiments:
X³ is a bond;

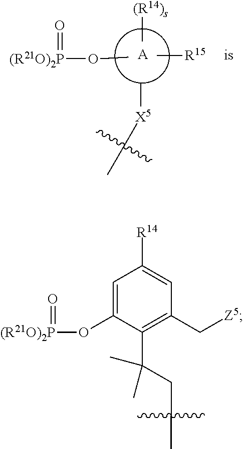

R¹⁴ is independently C₁₋₆ alkyl, C₂₋₆ alkenyl, C₁₋₆ haloalkyl, —C(O)R²³, —C(O)OR²³, or —C(O)N(R²⁴)(R²⁵), wherein each C₁₋₆ alkyl and C₂₋₆ alkenyl is optionally substituted with 1, 2, 3, or 4 Z⁵;
each R²¹ is independently H or C₁₋₄ alkyl;
each Z⁵ is independently —C(O)OR²³ or —C(O)N(R²⁴)(R²⁵);
each R²³ is independently hydrogen or C₁₋₆ alkyl, wherein each C₁₋₆ alkyl is optionally substituted with 1, 2, 3, or 4 Z⁶;
each R²⁴ and R²⁵ is independently hydrogen, C₁₋₆ alkyl, 6- to 10-membered aryl, or 5- to 10-membered heteroaryl, wherein each C₁₋₆ alkyl, 6- to 10-membered aryl, or 5- to 10-membered heteroaryl is optionally substituted with 1, 2, 3, or 4 Z⁶, or R²⁴ and R²⁵, together with the nitrogen to which they are attached form a 4 to 10-membered heterocyclyl optionally substituted with 1, 2, 3, or 4 Z⁶;
each Z⁶ is independently C₁₋₆ alkyl, —C(O)R²⁶, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —P(O)(OR²⁶)₂, —OP(O)(OR²⁶)₂, —P(O)(R²⁶)(OR²⁶), —OP(O)(R²⁶)(OR²⁶), —S(O)₂R²⁶, S(O)₂OR²⁶, or —S(O)₂N(R²⁷)(R²⁸) wherein each C₁₋₆ alkyl is optionally substituted with 1, 2, 3, or 4 Z⁷;
each Z⁷ is independently —C(O)R²⁶, —C(O)OR²⁶, or —C(O)N(R²⁷)(R²⁸);
each R²⁶ is independently hydrogen, C₁₋₆ alkyl, C₂₋₆ alkenyl, or C₂₋₆ alkynyl; and
each R²⁷ and R²⁸ is independently hydrogen, C₁₋₆ alkyl, C₂₋₆ alkenyl, or C₂₋₆ alkynyl.

In certain embodiments:
X³ is a bond;
each R^{x5} is C₁₋₄ alkyl, optionally substituted with 1 or 2 Z⁵;
q is 1 or 2;

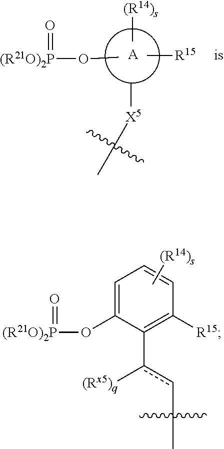

each R¹⁴ is independently C₁₋₆ alkyl or —C(O)OR²³, wherein each C₁₋₆ alkyl is optionally substituted with 1, 2, 3, or 4 Z⁵;
s is 0, 1, 2, or 3;
R¹⁵ is C₁₋₆ alkyl or C₂₋₆ alkenyl, each optionally substituted with 1 or 2 Z⁵;
each R²¹ is independently H or C₁₋₄ alkyl;
each Z⁵ is independently —C(O)OR²³ or —C(O)N(R²⁴)(R²⁵);
each R²³ is independently hydrogen or C₁₋₆ alkyl, wherein each C₁₋₆ alkyl is optionally substituted with 1, 2, 3, or 4 Z⁶;
each R²⁴ and R²⁵ is independently hydrogen, C₁₋₆ alkyl, 6- to 10-membered aryl, or 5- to 10-membered heteroaryl, wherein each C₁₋₆ alkyl, 6- to 10-membered aryl, or 5- to 10-membered heteroaryl is optionally substituted with 1, 2, 3, or 4 Z⁶, or R²⁴ and R²⁵, together with the nitrogen to which they are attached form a 4 to 10-membered heterocyclyl optionally substituted with 1, 2, 3, or 4 Z⁶;
each Z⁶ is independently C₁₋₆ alkyl, —C(O)R²⁶, —C(O)N(R²⁷)(R²⁸), —C(NOH)N(R²⁷)(R²⁸), —C(O)N(R²⁶)S(O)₂(R²⁶), —P(O)(OR²⁶)₂, —OP(O)(OR²⁶)₂, —P(O)(R²⁶)(OR²⁶), —S(O)₂R²⁶, S(O)₂OR²⁶, or —S(O)₂N(R²⁷)(R²⁸), wherein each C₁₋₆ alkyl is optionally substituted with 1 or 2 Z⁷;
each Z⁷ is —C(O)OR²⁶;
each R²⁶ is independently hydrogen, C₁₋₆ alkyl, C₂₋₆ alkenyl, or C₂₋₆ alkynyl; and
each R²⁷ and R²⁸ is independently hydrogen, C₁₋₆ alkyl, C₂₋₆ alkenyl, or C₂₋₆ alkynyl.

In certain embodiments:
X³ is a bond;

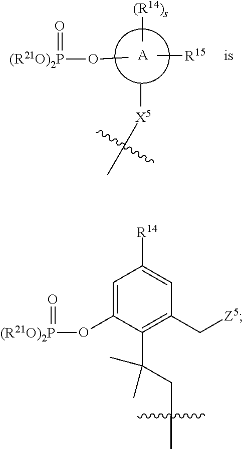

each R¹⁴ is independently C₁₋₆ alkyl or —C(O)OR²³, wherein each C₁₋₆ alkyl is optionally substituted with 1, 2, 3, or 4 Z⁵;
each R²¹ is independently H or C₁₋₄ alkyl;
each Z⁵ is independently —C(O)OR²³ or —C(O)N(R²⁴)(R²⁵);
each R²³ is independently hydrogen or C₁₋₆ alkyl, wherein each C₁₋₆ alkyl is optionally substituted with 1, 2, 3, or 4 Z⁶;
each R²⁴ and R²⁵ is independently hydrogen, C₁₋₆ alkyl, 6- to 10-membered aryl, or 5- to 10-membered heteroaryl, wherein each C₁₋₆ alkyl, 6- to 10-membered aryl, or 5- to 10-membered heteroaryl is optionally substituted with 1, 2, 3, or 4 Z⁶, or R²⁴ and R²⁵, together with the nitrogen to which they are attached form a 4 to 10-membered heterocyclyl optionally substituted with 1, 2, 3, or 4 Z⁶;
each Z⁶ is independently C₁₋₆ alkyl, —C(O)R²⁶, —C(O)OR²⁶, —C(O)N(R²⁷)(R²⁸), —C(NOH)N(R²⁷)(R²⁸), —C(O)N(R²⁶)S(O)₂(R²⁶), —P(O)(OR²⁶)₂, —OP(O)(OR²⁶)₂, —P(O)(R²⁶)(OR²⁶), —S(O)₂R²⁶, S(O)₂OR²⁶, or —S(O)₂N(R²⁷)(R²⁸), wherein each C₁₋₆ alkyl is optionally substituted with 1 or 2 Z⁷;
each Z⁷ is —C(O)OR²⁶;
each R²⁶ is independently hydrogen, C₁₋₆ alkyl, C₂₋₆ alkenyl, or C₂₋₆ alkynyl; and
   each R²⁷ and R²⁸ is independently hydrogen, C₁₋₆ alkyl, C₂₋₆ alkenyl, or C₂₋₆ alkynyl.

In certain embodiments, R² is C₁₋₄ alkyl. In certain embodiments, R² is C₃₋₆ cycloalkyl. In certain embodiments, R² is O—R^{2A}. In certain embodiments, R² is C₁₋₂ alkyl-O—R^{2A}, N—(R^{3A})₂. In certain embodiments, R² is C₁₋₂ alkyl-N—(R^{3A})₂.

In certain embodiments, R³ is C₁₋₄ alkyl. In certain embodiments, R³ is C₃₋₆ cycloalkyl. In certain embodiments, R³ is O—R^{2A}. In certain embodiments, R³ is C₁₋₂ alkyl-O—R^{2A}, N—(R^{3A})₂. In certain embodiments, R³ is C₁₋₂ alkyl-N—(R^{3A})₂.

In certain embodiments, R² and R³ are each independently C₁₋₄ alkyl, C₃₋₆ cycloalkyl, or O—R^{2A}, wherein R^{2A} is C₁₋₄ alkyl, C₃₋₆ cycloalkyl, or a 4 to 10-membered heterocyclyl having 1 to 5 heteroatoms selected from N, O, and S. In certain embodiments, at least one R² and R³ is C₁-4 alkyl, C₃₋₆ cycloalkyl, or O—R^{2A}, wherein R^{2A} is C₁₋₄ alkyl, C₃₋₆ cycloalkyl, or a 4 to 10-membered heterocyclyl having 1 to 5 heteroatoms selected from N, O, and S. In certain embodiments, R² and R³ are each independently:

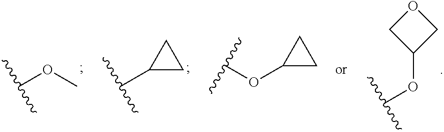

In certain embodiments, at least one of R² and R³ is:

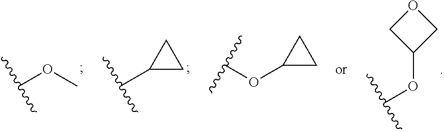

In certain embodiments, R² and R³ are each methoxy. In certain embodiments, at least one of R² and R³ is methoxy.

In certain embodiments, at least one R^{2A} is C₁₋₄ alkyl. In certain embodiments, at least one R^{2A} is C₃₋₆ cycloalkyl.

In certain embodiments, at least one R^{2A} is C₁₋₄ alkyl. In certain embodiments, at least one R^{2A} is C₃₋₆ cycloalkyl optionally substituted by 1 to 3 R^{f} groups. In certain embodiments, at least one R^{2A} is 4 to 10-membered heterocyclyl having 1 to 5 heteroatoms selected from N, O, and S. In certain embodiments, at least one R^{2A} is 4 to 10-membered heterocyclyl having 1 to 5 heteroatoms selected from N, O, and S optionally substituted by 1 to 3 R^{f} groups.

In certain embodiments, at least one R^{3A} is hydrogen. In certain embodiments, at least one R^{3A} is C₁₋₄ alkyl. In certain embodiments, at least one R^{3A} is C₃₋₆ cycloalkyl. In certain embodiments, at least one R^{3A} is C₃₋₆ cycloalkyl optionally substituted by 1 to 3 R^{f} groups. In certain embodiments, at least one R^{3A} is COO(R^{e}).

In certain embodiments, at least one R^{e} is hydrogen. In certain embodiments, at least one R^{e} is C₁₋₄ alkyl.

In certain embodiments, at least one R^{f} is C₁₋₂ alkyl. In certain embodiments, at least one R^{f} is halogen.

In certain embodiments, R⁴ is hydrogen. In certain embodiments, R⁴ is halo. In certain embodiments, R⁴ is C₁₋₄ alkyl. In certain embodiments, R⁴ is C₁₋₄ haloalkyl. In certain embodiments, R⁴ is C₃₋₆ cycloalkyl. In certain embodiments, R⁴ is C₁₋₄ alkoxy. In certain embodiments, R⁴ is C₁₋₄ haloalkoxy.

In certain embodiments, R⁴ is hydrogen, C₁₋₄ alkyl, or C₁₋₄ haloalkyl. In certain embodiments, R⁴ is C₁₋₄ haloalkyl. In certain embodiments, R⁴ is CF₃.

In certain embodiments, R⁷ is hydrogen. In certain embodiments, R⁷ is halo. In certain embodiments, R⁷ is C₁₋₄ alkyl. In certain embodiments, R⁷ is C₁₋₄ haloalkyl. In certain embodiments, R⁷ is C₃₋₆ cycloalkyl. In certain embodiments, R⁷ is C₁₋₄ alkoxy. In certain embodiments, R⁷ is C₁₋₄ haloalkoxy.

In certain embodiments, R⁷ is hydrogen, C₁₋₄ alkyl, or C₁₋₄ haloalkyl. In certain embodiments, R⁷ is C₁₋₄ haloalkyl. In certain embodiments, R⁷ is CF₃.

In certain embodiments, R⁵ is hydrogen. In certain embodiments, R⁵ is halo. In certain embodiments, R⁵ is C₁₋₂ alkyl. In certain embodiments, R⁵ is C₁₋₂haloalkyl. In certain embodiments, R⁵ is C₃₋₆ cycloalkyl.

In certain embodiments, R⁶ is hydrogen. In certain embodiments, R⁶ is halo. In certain embodiments, R⁶ is C₁₋₂ alkyl. In certain embodiments, R⁶ is C₁₋₂haloalkyl. In certain embodiments, R⁶ is C₃₋₆ cycloalkyl.

In certain embodiments, R⁵ and R⁶ are each C₁₋₂ alkyl. In certain embodiments, at least one of R⁵ and R⁶ is C₁₋₂ alkyl. In certain embodiments, R⁵ and R⁶ are each methyl. In certain embodiments, at least one of R⁵ and R⁶ is methyl.

In certain embodiments, R⁸ is hydrogen. In certain embodiments, R⁸ is halo. In certain embodiments, R⁸ is C₁₋₂ alkyl. In certain embodiments, R⁸ is C₁₋₂haloalkyl. In certain embodiments, R⁸ is C₃₋₆ cycloalkyl.

In certain embodiments, R⁹ is hydrogen. In certain embodiments, R⁹ is halo. In certain embodiments, R⁹ is C₁₋₂ alkyl. In certain embodiments, R⁹ is C₁₋₂haloalkyl. In certain embodiments, R⁹ is C₃₋₆ cycloalkyl.

In certain embodiments, R⁸ and R⁹ are each C₁₋₂ alkyl. In certain embodiments, at least one of R⁸ and R⁹ is C₁₋₂ alkyl. In certain embodiments, R⁸ and R⁹ are each methyl. In certain embodiments, at least one of R⁸ and R⁹ is methyl.

In certain embodiments, n is 0. In certain embodiments, n is 1. In certain embodiments, n is 2. In certain embodiments, n is 3. In certain embodiments, n is 4.

In certain embodiments, each R¹⁰ is halogen. In certain embodiments, each R¹⁰ is cyano. In certain embodiments, each R¹⁰ is C₁₋₄ alkoxy. In certain embodiments, each R¹⁰ is C₁₋₆ alkyl. In certain embodiments, each R¹⁰ is C₃₋₆ cycloalkyl. In certain embodiments, at least one R¹⁰ is halogen. In certain embodiments, at least one R¹⁰ is cyano. In certain embodiments, at least one R¹⁰ is C₁₋₄ alkoxy. In certain embodiments, at least one R¹⁰ is C₁₋₆ alkyl. In certain embodiments, at least one R¹⁰ is C₃₋₆ cycloalkyl.

In certain embodiments, each R¹⁰ is halogen. In certain embodiments, at least one R¹⁰ is halogen. In certain embodiments, each R¹⁰ is fluoro. In certain embodiments, at least one R¹⁰ is fluoro.

In certain embodiments, A is ethynyl. In certain embodiments, A is bond.

In certain embodiments, R¹ is a 5- to 6-membered heterocyclyl having 1 to 3 heteroatoms selected from N, O, and S, or a 5 to 6-membered heteroaryl having 1 to 3 heteroatoms selected from N, O, and S, wherein the 5 to 6-membered heterocyclyl or 5 to 6-membered heteroaryl is optionally substituted with 1 to 3 R^{a} groups. In certain embodiments, R¹ is a 5- to 6-membered heteroaryl having 1 to 3 heteroatoms selected from N, O, and S and is optionally substituted with 1 to 3 R^{a} groups. In certain embodiments, R¹ is a 5- to 6-membered heteroaryl having 1 to 3 heteroatoms selected from N, O, and S and is substituted with 1 to 3 R^{a} groups. In certain embodiments, R¹ is a 5- to 6-membered heteroaryl having 1 to 3 heteroatoms selected from N, O, and S. In certain embodiments, R¹ is independently:

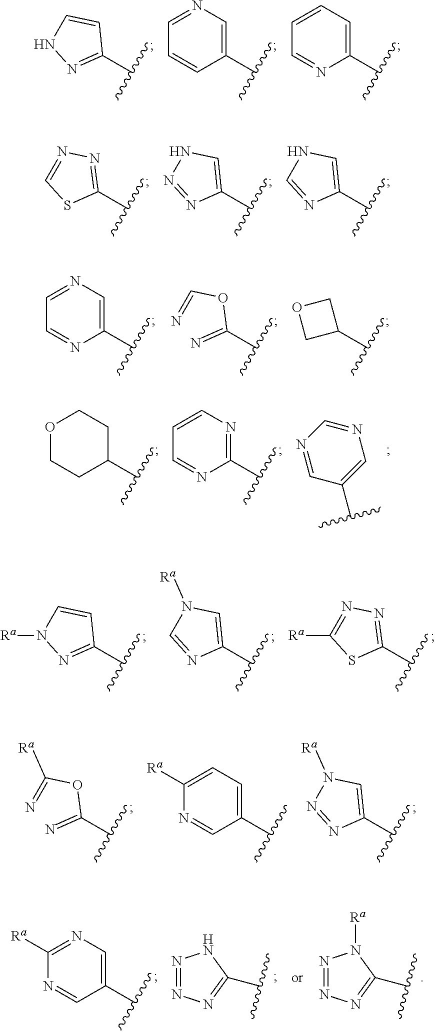

In certain embodiments, R¹ is:

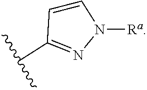

In certain embodiments, each R^{a} is halogen. In certain embodiments, each R^{a} is C₁₋₄ alkyl. In certain embodiments, each R^{a} is C₁₋₄ alkyl with 1 to 2 groups selected from hydroxyl and C₁₋₄ alkoxy, C₁₋₄ haloalkyl, C₁₋₄ alkoxy, C₃₋₆ cycloalkyl, 4 to 10-membered heterocyclyl having 1 to 5 heteroatoms selected from N, O, and S which is optionally substituted with Rai. In certain embodiments, each R^{a} is O—R^{3B}.

In certain embodiments, R^{a} is C₁₋₄ haloalkyl. In certain embodiments, R^{a} is:

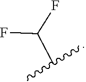 [SMILES: *C(F)F]

In certain embodiments, R^{3B} is C₃₋₆ cycloalkyl. In certain embodiments, R^{3B} is C₃₋₆ cycloalkyl optionally substituted with R^{a1} or a 4 to 10-membered heterocyclyl having 1 to 5 heteroatoms selected from N, O, and S optionally substituted with R^{a1}.

In certain embodiments, R^{a1} is C₁₋₄ alkyl. In certain embodiments, C₃₋₆ cycloalkyl. In certain embodiments, C₁₋₄ haloalkyl. In certain embodiments, 4 to 8-membered heterocyclyl having 1 to 3 heteroatoms selected from N, O, and S.

In certain embodiments, X¹ is a 6-membered aryl or a 5 to 6-membered heteroaryl having 1 to 3 heteroatoms selected from N, O, and S, wherein each 6-membered aryl or 5 to 6-membered heteroaryl is optionally substituted with 1 to 4 R^{b} groups. In certain embodiments, X¹ is a 6-membered aryl or a 5 to 6-membered heteroaryl having 1 to 3 heteroatoms selected from N, O, and S, wherein each 6-membered aryl or 5 to 6-membered heteroaryl is substituted with 1 to 4 R^{b} groups. In certain embodiments, X¹ is a 6-membered aryl or a 5 to 6-membered heteroaryl having 1 to 3 heteroatoms selected from N, O, and S, wherein each 6-membered aryl or 5 to 6-membered heteroaryl. In certain embodiments, X¹ is:

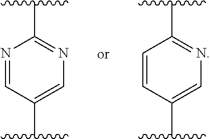 [SMILES: *c1cnc(*)nc1 ; *c1ccc(*)nc1]

In certain embodiments, X¹ is

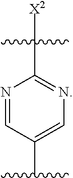

In certain embodiments, X² is hydrogen. In certain embodiments, X² is a 4 to 10-membered heterocyclyl having 1 to 3 heteroatoms selected from N, O, and S and is optionally substituted with one R¹¹ and optionally substituted with 1 to 5 R^{b} groups. In certain embodiments, X² is a 4 to 10-membered heterocyclyl having 1 to 3 heteroatoms selected from N, O, and S and is substituted with one R¹¹ and optionally substituted with 1 to 5 R^{b} groups. In certain embodiments, X² is a 4 to 10-membered heterocyclyl having 1 to 3 heteroatoms selected from N, O, and S and is optionally substituted with one R¹¹ and substituted with 1 to 5 R^{b} groups. In certain embodiments, X² is a 4 to 10-membered heterocyclyl having 1 to 3 heteroatoms selected from N, O, and S and is substituted with one R¹¹ and substituted with 1 to 5 R^{b} groups. In certain embodiments, X² is a 4 to 10-membered heterocyclyl having 1 to 3 heteroatoms selected from N, O, and S. In certain embodiments, X² is:

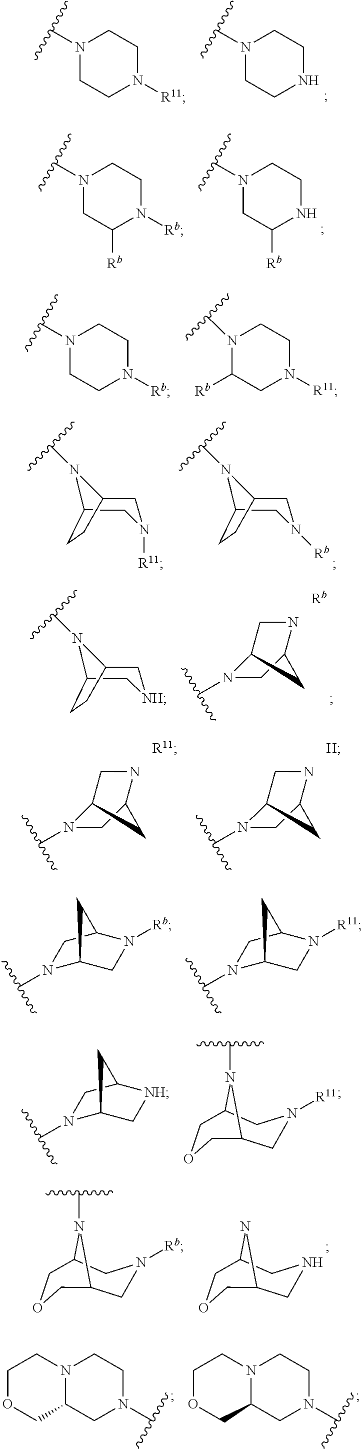

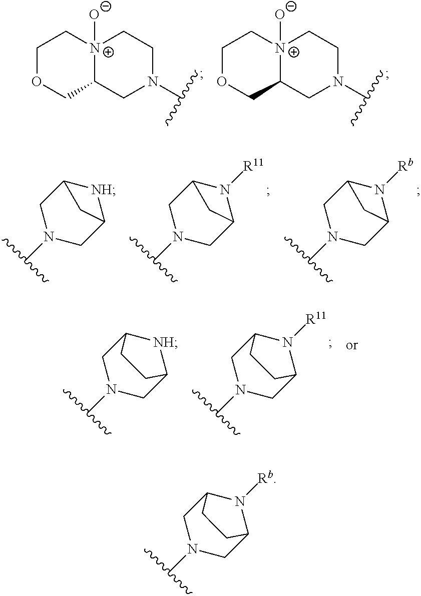

In certain embodiments, X² is:

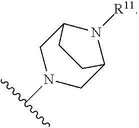

In certain embodiments, R¹¹ is —C═O(R^{c}). In certain embodiments, R¹¹ is CH₂(R^{d}). In certain embodiments, R¹¹ is S(O)₁₋₂(C₁₋₄ alkyl). In certain embodiments, R¹¹ is S(O)₁₋₂—(C₃₋₆ cycloalkyl). In certain embodiments, R¹¹ is 4 to 10-membered heterocyclyl having 1 to 5 heteroatoms selected from N, O, and S. In certain embodiments, R¹¹ is 5 to 9-membered heteroaryl having 1 to 5 heteroatoms selected from N, O, and S, wherein each 4 to 10-membered heterocyclyl. In certain embodiments, R¹¹ is 5 to 9-membered heteroaryl is optionally substituted with 1 to 5 R^{b} groups.

In certain embodiments, R¹¹ is 4 to 10-membered heterocyclyl having 1 to 3 heteroatoms selected from N, O, and S. In certain embodiments, R¹¹ is a 4 to 6-membered heterocyclyl having one oxygen. In certain embodiments, R¹¹ is oxetan-3-yl.

In certain embodiments, each R^{b} is halogen. In certain embodiments, each R^{b} is oxo. In certain embodiments, each R^{b} is C₁₋₄ alkyl. In certain embodiments, each R^{b} is C₁₋₄ alkyl with 1 to 2 groups selected from hydroxyl and C₁₋₄ alkoxy. In certain embodiments, each R^{b} is C₁₋₄ haloalkyl. In certain embodiments, each R^{b} is C₁₋₄ alkoxy. In certain embodiments, each R^{b} is COO(R^{e}).

In certain embodiments, R^{c} is C₁₋₄ alkyl. In certain embodiments, R^{c} is C₁₋₄ haloalkyl. In certain embodiments, R^{c} is C₁₋₄ alkoxy. In certain embodiments, R^{c} is N(R^{e})₂. In certain embodiments, R^{c} is C₃₋₆ cycloalkyl. In certain embodiments, R^{c} is C₃₋₆ cycloalkyl substituted by 1 to 5 R^{b} groups. In certain embodiments, R^{c} is 4 to 6-membered heterocyclyl having 1 to 3 heteroatoms selected from N, O, and S. In certain embodiments, R^{c} is 4 to 6-membered heterocyclyl having 1 to 3 heteroatoms selected from N, O, and S substituted by 1 to 5 R^{b} groups.

In certain embodiments, R^{d} is COO(R^{e}). In certain embodiments, R^{d} is N(R^{e})₂. In certain embodiments, R^{d} is C₃₋₆ cycloalkyl. In certain embodiments, R^{d} is C₃₋₆ cycloalkyl substituted by 1 to 5 R^{b} groups. In certain embodiments, R^{d} is 4 to 6-membered heterocyclyl having 1 to 3 heteroatoms selected from N, O, and S. In certain embodiments, R^{d} is 4 to 6-membered heterocyclyl having 1 to 3 heteroatoms selected from N, O, and S substituted by 1 to 5 R^{b} groups;

In certain embodiments, each R¹² is C₁₋₂ alkyl. In certain embodiments, each R¹² is halo. In certain embodiments, each R¹² is —OC₁₋₂ alkyl. In certain embodiments, each R¹² is cyano.

In certain embodiments, p is 0. In certain embodiments, p is 1. In certain embodiments, p is 2. In certain embodiments, p is 3. In certain embodiments, p is 4.

In certain embodiments, provided herein is a compound of Formula (II):

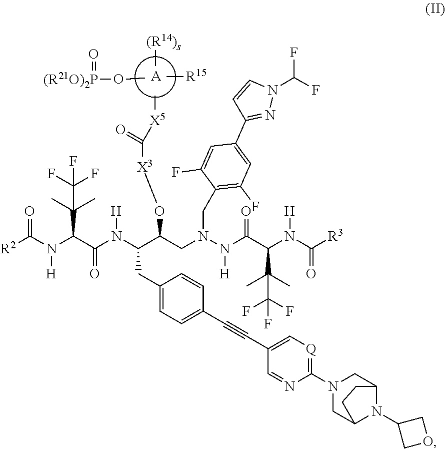

or a pharmaceutically acceptable salt thereof, wherein Q is N or CH, and R², R³, R¹⁴, R¹⁵, R²¹, X³, X⁵, ring \textcircled{A}, and s are as described herein.

In certain embodiments of a compound of Formula (II), or a pharmaceutically acceptable salt thereof, Q is N. In certain embodiments of a compound of Formula (II), or a pharmaceutically acceptable salt thereof, Q is CH.

In certain embodiments, the is a compound of Formula (III):

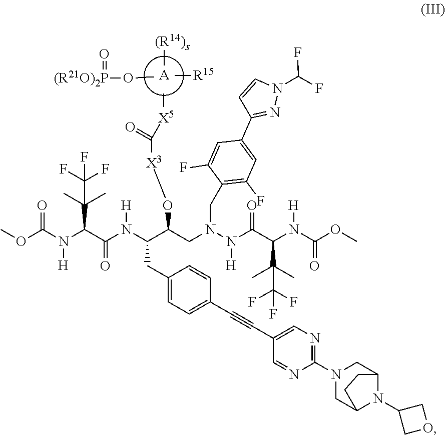

or a pharmaceutically acceptable salt thereof, and R¹⁴, R¹⁵, R²¹, X³, X⁵, ring \textcircled{A}, and s are as described herein.

In certain embodiments, a compound of any of the formulae described herein, or a pharmaceutically acceptable salt thereof, has the variables as described herein.

In certain embodiments, X³ is —C(O)O—X⁴—O—. In certain embodiments, X³ is bond.

In certain embodiments, X⁴ is C₁₋₆ alkyl optionally substituted with 1, 2, or 3 R^{x4}In certain embodiments, X⁴ is C₁₋₆ alkyl. In certain embodiments, X⁴ is C₁₋₆ alkyl substituted with 1, 2, or 3 R^{x4}.

In certain embodiments, X⁴ is C₁₋₃ alkyl optionally substituted with 1 or 2 R^{x4}. In certain embodiments, X⁴ is C₁₋₃ alkyl. In certain embodiments, X⁴ is C₁₋₃ alkyl substituted with 1 or 2 R^{x4}.

In certain embodiments, each R^{x4} is independently C₁₋₆ alkyl optionally substituted with 1, 2, 3, or 4 Z⁵. In certain embodiments, each R^{x4} is independently C₁₋₆ alkyl. In certain embodiments, each R^{x4} is independently C₁₋₆ alkyl substituted with 1, 2, 3, or 4 Z⁵.

In certain embodiments, at least one R^{x4} is independently C₁₋₆ alkyl optionally substituted with 1, 2, 3, or 4 Z⁵. In certain embodiments, at least one R^{x4} is independently C₁₋₆ alkyl. In certain embodiments, at least one R^{x4} is independently C₁₋₆ alkyl substituted with 1, 2, 3, or 4 Z⁵.

In certain embodiments, X³ is a bond.

In certain embodiments, X⁵ is C₁₋₆ alkyl optionally substituted with 1, 2, 3, or 4 R^{x5}. In certain embodiments, X⁵ is C₁₋₆ alkyl. In certain embodiments, X⁵ is C₁₋₆ alkyl substituted with 1, 2, 3, or 4 R^{x5}.

In certain embodiments, X⁵ is C₁₋₃ alkyl optionally substituted with 1 or 2 R^{x5}. In certain embodiments, X⁵ is C₁₋₃ alkyl. In certain embodiments, X⁵ is C₁₋₃ alkyl substituted with 1 or 2 R^{x5}.

In certain embodiments, ring \textcircled{A} is 6-membered aryl or 5 to 6-membered heteroaryl, and s is 0, 1, 2, 3, or 4. In certain embodiments, ring \textcircled{A} is 6-membered aryl, and s is 0, 1, 2, or 3. In certain embodiments, ring \textcircled{A} is 5 to 6-membered heteroaryl, and s is 0, 1, or 2. In certain embodiments, ring \textcircled{A} is 5-membered heteroaryl, and s is 0, 1, or 2. In certain embodiments, ring \textcircled{A} is 6-membered heteroaryl, and s is 0, 1, or 2.

In certain embodiments, the compound is a compound of Formula (IV):

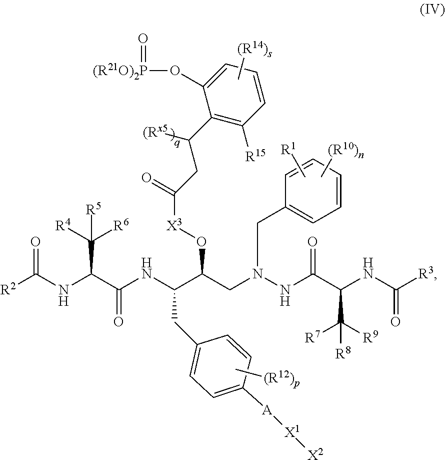

or a pharmaceutically acceptable salt thereof, wherein q is 0, 1 or 2, and R¹, R², R³, R⁴, R⁵, R⁶, R⁷, R⁸, R⁹, R¹⁰, R¹², R¹⁴, R¹⁵, R²¹, X¹, X², X³, R^{x5}, A, n, p, q, and s are as described herein.

In certain embodiments of a compound of Formula (IV), or a pharmaceutically acceptable salt thereof, q is 0. In certain embodiments of a compound of Formula (IV), or a pharmaceutically acceptable salt thereof, q is 1. In certain embodiments of a compound of Formula (IV), or a pharmaceutically acceptable salt thereof, q is 2.

In certain embodiments, the compound is a compound of Formula (V):

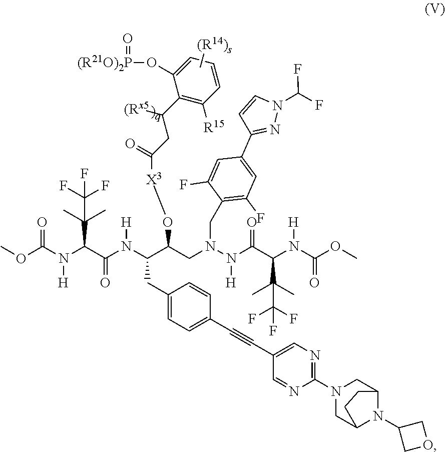

or a pharmaceutically acceptable salt thereof, wherein q is 0, 1 or 2, and R¹⁴, R¹⁵, R²¹, X³, R^{x5}, q, and s are as described herein.

In certain embodiments of a compound of Formula (V), or a pharmaceutically acceptable salt thereof, q is 0. In certain embodiments of a compound of Formula (V), or a pharmaceutically acceptable salt thereof, q is 1. In certain embodiments of a compound of Formula (V), or a pharmaceutically acceptable salt thereof, q is 2.

In certain embodiments, s is 0. In certain embodiments, s is 1.

In certain embodiments, the compound is a compound of Formula (VI):

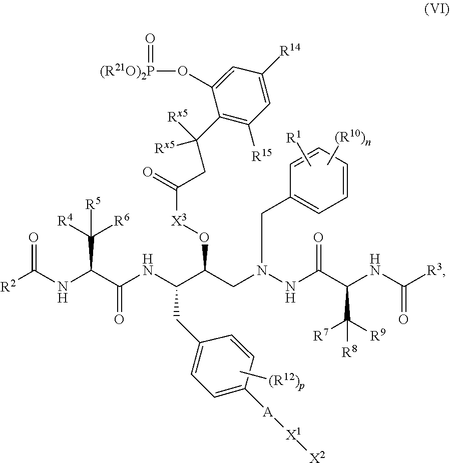

or a pharmaceutically acceptable salt thereof, and R¹, R², R³, R⁴, R⁵, R⁶, R⁷, R⁸, R⁹, R¹⁰, R¹², R¹⁴, R¹⁵, R²¹, X¹, X², X³, R^{x5}, A, n, and p are as described herein.

In certain embodiments, the compound is a compound of Formula (VII):

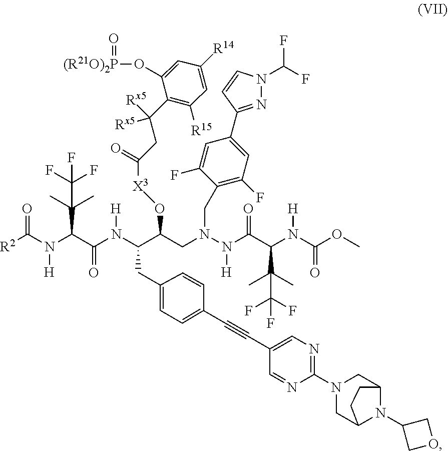

or a pharmaceutically acceptable salt thereof, and R¹⁴, R¹⁵, R²¹, X³, and R^{x5} are as described herein.

In certain embodiments, each R^{x5} is independently C₁₋₆ alkyl, C₂₋₆ alkenyl, C₁₋₆ haloalkyl, or —OR²³, and each C₁₋₆ alkyl and C₂₋₆ alkenyl is optionally substituted with 1, 2, 3, or 4 Z⁵. In certain embodiments, each R^{x5} is independently C₁₋₆ alkyl, C₂₋₆ alkenyl, C₁₋₆ haloalkyl, or —OR²³. In certain embodiments, at least one R^{x5} is independently C₁₋₆ alkyl, C₂₋₆ alkenyl, C₁₋₆ haloalkyl, or —OR²³, and each C₁₋₆ alkyl and C₂₋₆ alkenyl is optionally substituted with 1, 2, 3, or 4 Z⁵. In certain embodiments, at least one R^{x5} is independently C₁₋₆ alkyl, C₂₋₆ alkenyl, C₁₋₆ haloalkyl, or —OR²³.

In certain embodiments, each R^{x5} is independently C₁₋₃ alkyl optionally substituted with 1 or 2 Z⁵. In certain embodiments, each R^{x5} is independently C₁₋₃ alkyl. In certain embodiments, each R^{x5} is independently C₁₋₃ alkyl substituted with 1 or 2 Z⁵.

In certain embodiments, at least one R^{x5} is independently C₁₋₃ alkyl optionally substituted with 1 or 2 Z⁵. In certain embodiments, at least one R^{x5} is independently C₁₋₃ alkyl. In certain embodiments, at least one R^{x5} is independently C₁₋₃ alkyl substituted with 1 or 2 Z⁵.

In certain embodiments, each R^{x5} is methyl. In certain embodiments, at least one R^{x5} is methyl.

In certain embodiments, R¹⁵ is —C(O)OR²³.

In certain embodiments, R¹⁵ is C₁₋₆ alkyl, C₂₋₆ alkenyl, C₁₋₆ haloalkyl, —C(O)R²³, —C(O)OR²³, or —C(O)N(R²⁴)(R²⁵), and each C₁₋₆ alkyl and C₂₋₆ alkenyl is optionally substituted with 1, 2, 3, or 4 Z⁵.

In certain embodiments, R¹⁵ is C₁₋₆ alkyl, C₂₋₆ alkenyl, C₁₋₆ haloalkyl, —C(O)R²³, —C(O)OR²³, or —C(O)N(R²⁴)(R²⁵).

In certain embodiments, R¹⁵ is C₁₋₆ alkyl or C₂₋₆ alkenyl, each optionally substituted with 1 or 2 Z⁵. In certain embodiments, R¹⁵ is C₁₋₆ alkyl or C₂₋₆ alkenyl. In certain embodiments, R¹⁵ is C₁₋₆ alkyl or C₂₋₆ alkenyl, each substituted with 1 or 2 Z⁵.

In certain embodiments, R¹⁵ is C₁₋₃ alkyl substituted with one Z⁵.

In certain embodiments, the compound is a compound of Formula (VIII):

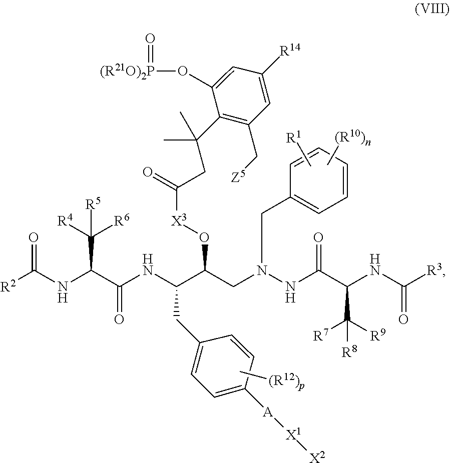

or a pharmaceutically acceptable salt thereof, and R¹, R², R³, R⁴, R⁵, R⁶, R⁷, R⁸, R⁹, R¹⁰, R¹², R¹⁴, R²¹, X¹, X², X³, X⁵, Z⁵, A, n, and p are as described herein.

In certain embodiments, the compound is a compound of Formula (IX):

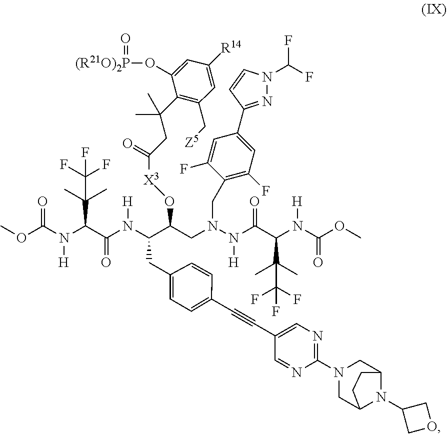

or a pharmaceutically acceptable salt thereof, and R¹⁴, R²¹, X³, and Z⁵ are as described herein.

In certain embodiments, Z⁵ is independently —C(O)OR²³ or —C(O)N(R²⁴)(R²⁵).

In certain embodiments, Z⁵ is —C(O)N(R²⁴)(R²⁵).

In certain embodiments, the compound is a compound of Formula (X):

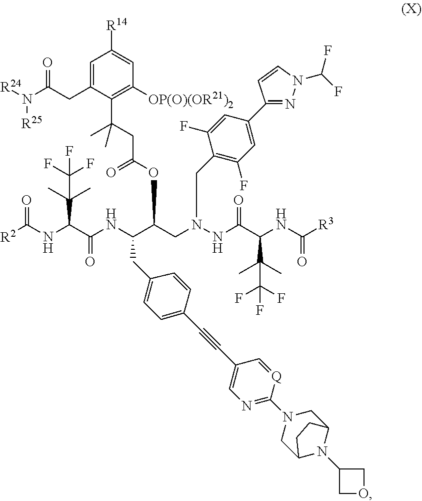

or a pharmaceutically acceptable salt thereof, wherein Q is N or CH, and R², R³, R¹⁴, R²¹, R²⁴, and R²⁵ are as described herein.

In certain embodiments of a compound of Formula (X), or a pharmaceutically acceptable salt thereof, Q is N. In certain embodiments of a compound of Formula (X), or a pharmaceutically acceptable salt thereof, Q is CH.

In certain embodiments of a compound of Formula (X), or a pharmaceutically acceptable salt thereof, R² and R³ are each independently C₁₋₄ alkyl, C₃₋₆ cycloalkyl, or O—R^{2A}, wherein R^{2A} is C₁₋₄ alkyl, C₃₋₆ cycloalkyl, or a 4 to 10-membered heterocyclyl having 1 to 5 heteroatoms selected from N, O, and S.

In certain embodiments of a compound of Formula (X), or a pharmaceutically acceptable salt thereof, R² and R³ are each independently:

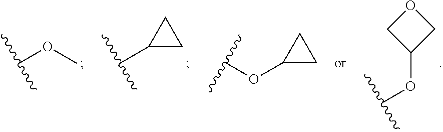

In certain embodiments of a compound of Formula (X), or a pharmaceutically acceptable salt thereof, R² and R³ are each methoxy.

In certain embodiments, the compound is a compound of Formula (XI):

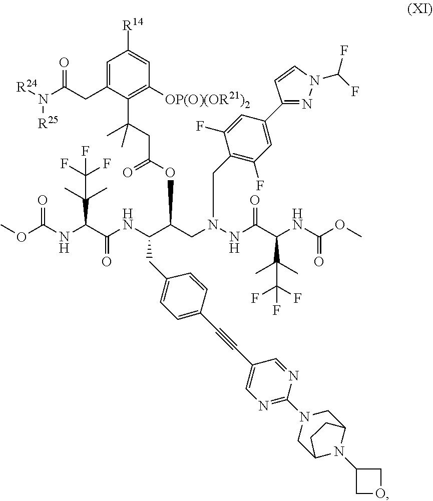

or a pharmaceutically acceptable salt thereof, and R¹⁴, R²¹, R²⁴, and R²⁵ are as described herein.

In certain embodiments, each R²¹ is hydrogen. In certain embodiments, at least one R²¹ is hydrogen.

In certain embodiments, each R¹⁴ is independently C₁₋₆ alkyl, C₂₋₆ alkenyl, C₁₋₆ haloalkyl, —C(O)R²³, —C(O)OR²³, or —C(O)N(R²⁴)(R²⁵), and each C₁₋₆ alkyl is optionally substituted with 1, 2, 3, or 4 Z⁵.

In certain embodiments, each R¹⁴ is independently C₁₋₆ alkyl, C₂₋₆ alkenyl, C₁₋₆ haloalkyl, —C(O)R²³, —C(O)OR²³, or —C(O)N(R²⁴)(R²⁵). In certain embodiments, at least one R¹⁴ is independently C₁₋₆ alkyl, C₂₋₆ alkenyl, C₁₋₆ haloalkyl, —C(O)R²³, —C(O)OR²³, or —C(O)N(R²⁴)(R²⁵), and each C₁₋₆ alkyl is optionally substituted with 1, 2, 3, or 4 Z⁵.

In certain embodiments, at least one R¹⁴ is independently C₁₋₆ alkyl, C₂₋₆ alkenyl, C₁₋₆ haloalkyl, —C(O)R²³, —C(O)OR²³, or —C(O)N(R²⁴)(R²⁵).

In certain embodiments, each R¹⁴ is independently C₁₋₆ alkyl or —C(O)OR²³, and each C₁₋₆ alkyl is optionally substituted with 1, 2, 3, or 4 Z⁵. In certain embodiments, each R¹⁴ is independently C₁₋₆ alkyl or —C(O)OR²³. In certain embodiments, at least one R¹⁴ is independently C₁₋₆ alkyl or —C(O)OR²³, and each C₁₋₆ alkyl is optionally substituted with 1, 2, 3, or 4 Z⁵. In certain embodiments, at least one R¹⁴ is independently C₁₋₆ alkyl or —C(O)OR²³.

In certain embodiments, each R²³ is independently hydrogen or C₁₋₆ alkyl, and each C₁₋₆ alkyl is optionally substituted with 1, 2, 3, or 4 Z⁶. In certain embodiments, each R²³ is independently hydrogen or C₁₋₆ alkyl. In certain embodiments, at least one R²³ is independently hydrogen or C₁₋₆ alkyl, and each C₁₋₆ alkyl is optionally substituted with 1, 2, 3, or 4 Z⁶. In certain embodiments, at least one R²³ is independently hydrogen or C₁₋₆ alkyl.

In certain embodiments, each R²⁴ and R²⁵ is independently hydrogen, C₁₋₆ alkyl, aryl, or heteroaryl, and each C₁₋₆ alkyl, aryl, and heteroaryl is optionally substituted with 1, 2, 3, or 4 Z⁶, or R²⁴ and R²⁵, together with the nitrogen to which they are attached, form a 4 to 10-membered heterocyclyl optionally substituted with 1, 2, 3, or 4 Z⁶. In certain embodiments, each R²⁴ and R²⁵ is independently hydrogen, C₁₋₆ alkyl, aryl, or heteroaryl. In certain embodiments, at least one of R²⁴ and R²⁵ is independently hydrogen, C₁₋₆ alkyl, aryl, or heteroaryl, and each C₁₋₆ alkyl, aryl, and heteroaryl is optionally substituted with 1, 2, 3, or 4 Z⁶, or R²⁴ and R²⁵, together with the nitrogen to which they are attached, form a 4 to 10-membered heterocyclyl optionally substituted with 1, 2, 3, or 4 Z⁶. In certain embodiments, at least one of R²⁴ and R²⁵ is independently hydrogen, C₁₋₆ alkyl, aryl, or heteroaryl. In certain embodiments, R²⁴ and R²⁵, together with the nitrogen to which they are attached, form a 4 to 10-membered heterocyclyl optionally substituted with 1, 2, 3, or 4 Z⁶.

In certain embodiments, each R²⁴ and R²⁵ is independently hydrogen or C₁₋₆ alkyl optionally substituted with 1 or 2 Z⁶. In certain embodiments, each R²⁴ and R²⁵ is independently hydrogen or C₁₋₆ alkyl. In certain embodiments, at least one of R²⁴ and R²⁵ is independently hydrogen or C₁₋₆ alkyl optionally substituted with 1 or 2 Z⁶. In certain embodiments, at least one of R²⁴ and R²⁵ is independently hydrogen or C₁₋₆ alkyl.

In certain embodiments, each R²⁴ and R²⁵ is independently hydrogen, 6-membered aryl, or 5 to 6-membered heteroaryl, each optionally substituted with 1 or 2 Z⁶. In certain embodiments, each R²⁴ and R²⁵ is independently hydrogen, 6-membered aryl, or 5 to 6-membered heteroaryl. In certain embodiments, at least one of R²⁴ and R²⁵ is independently hydrogen, 6-membered aryl, or 5 to 6-membered heteroaryl, each optionally substituted with 1 or 2 Z⁶. In certain embodiments, at least one of R²⁴ and R²⁵ is independently hydrogen, 6-membered aryl, or 5 to 6-membered heteroaryl.

In certain embodiments, each R²⁴ and R²⁵, together with the nitrogen to which they are attached, form a 5 or 6-membered heterocyclyl optionally substituted with 1 or 2 Z⁶. In certain embodiments, each R²⁴ and R²⁵, together with the nitrogen to which they are attached, form a 5 or 6-membered heterocyclyl. In certain embodiments, at least one of R²⁴ and R²⁵, together with the nitrogen to which they are attached, form a 5 or 6-membered heterocyclyl optionally substituted with 1 or 2 Z⁶. In certain embodiments, at least one of R²⁴ and R²⁵, together with the nitrogen to which they are attached, form a 5 or 6-membered heterocyclyl.

In certain embodiments, each Z⁶ is independently C₁₋₆ alkyl, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —P(O)(OR²⁶)₂, —OP(O)(OR²⁶)₂, —P(O)(R²⁶)(OR²⁶), —OP(O)(R²⁶)(OR²⁶), —S(O)₂R²⁶, S(O)₂OR²⁶, or —S(O)₂N(R²⁷)(R²⁸), and each C₁₋₆ alkyl is optionally substituted with 1 or 2 Z⁷. In certain embodiments, each Z⁶ is independently C₁₋₆ alkyl, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —P(O)(OR²⁶)₂, —OP(O)(OR²⁶)₂, —P(O)(R²⁶)(OR₂₆), —OP(O)(R²⁶)(OR²⁶), —S(O)₂R²⁶, S(O)₂OR²⁶, or —S(O)₂N(R²⁷)(R²⁸). In certain embodiments, at least one Z⁶ is independently C₁₋₆ alkyl, —C(O)OR²⁶, —C(O)N(R²⁷)(R²⁸) C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —P(O)(OR²⁶)₂, —OP(O)(OR²⁶)₂, —P(O)(R²⁶)(OR₂₆), —OP(O)(R²⁶)(OR²⁶), —S(O)₂R²⁶, S(O)₂OR²⁶, or —S(O)₂N(R²⁷)(R²⁸), and each C₁₋₆ alkyl is optionally substituted with 1 or 2 Z⁷. In certain embodiments, at least one Z⁶ is independently C₁₋₆ alkyl, —C(O)OR²⁶, —C(O)N(R²⁷)(R²⁸), —C(NOH)N(R²⁷)(R²⁸), —C(O)N(R²⁶)S(O)₂(R²⁶), —C(O)N(R²⁶)S(O)₂N(R²⁷)(R²⁸), —C(O)N(R²⁶)S(O)₂O(R²⁶), —P(O)(OR²⁶)₂, —OP(O)(OR²⁶)₂, —P(O)(R²⁶)(OR₂₆), —OP(O)(R²⁶)(OR²⁶), —S(O)₂R²⁶, S(O)₂OR²⁶, or —S(O)₂N(R²⁷)(R²⁸).

In certain embodiments, each Z⁶ is independently C₁₋₆ alkyl, —C(O)OR²⁶, —C(O)N(R²⁷)(R²⁸), —C(NOH)N(R²⁷)(R²⁸), —C(O)N(R²⁶)S(O)₂(R²⁶), —P(O)(OR²⁶)₂, —OP(O)(OR²⁶)₂, —S(O)₂R²⁶, S(O)₂OR²⁶, or —S(O)₂N(R²⁷)(R²⁸), and each C₁₋₆ alkyl is optionally substituted with 1 or 2 Z⁷. In certain embodiments, each Z⁶ is independently C₁₋₆ alkyl, —C(O)OR²⁶, —C(O)N(R²⁷)(R²⁸), —C(NOH)N(R²⁷)(R²⁸), —C(O)N(R²⁶)S(O)₂(R²⁶), —P(O)(OR²⁶)₂, —OP(O)(OR²⁶)₂, —S(O)₂R²⁶, S(O)₂OR²⁶, or —S(O)₂N(R²⁷)(R²⁸). In certain embodiments, at least one Z⁶ is independently C₁₋₆ alkyl, —C(O)OR²⁶, —C(O)N(R²⁷)(R²⁸), —C(NOH)N(R²⁷)(R²⁸) C(O)N(R²⁶)S(O)₂(R²⁶), —P(O)(OR²⁶)₂, —OP(O)(OR²⁶)₂, —S(O)₂R²⁶, S(O)₂OR²⁶, or —S(O)₂N(R²⁷)(R²⁸), and each C₁₋₆ alkyl is optionally substituted with 1 or 2 Z⁷. In certain embodiments, at least one Z⁶ is independently C₁₋₆ alkyl, —C(O)OR²⁶, —C(O)N(R²⁷)(R²⁸) C(NOH)N(R²⁷)(R²⁸), —C(O)N(R²⁶)S(O)₂(R²⁶), —P(O)(OR²⁶)₂, —OP(O)(OR²⁶)₂, —S(O)₂R²⁶, S(O)₂OR26, or —S(O)₂N(R²⁷)(R²⁸).

In certain embodiments, each Z⁶ is independently —C(O)OR²⁶ or —S(O)₂OR²⁶. In certain embodiments, at least one Z⁶ is independently —C(O)OR²⁶ or —S(O)₂OR²⁶.

In certain embodiments, each Z⁶ is independently —C(O)OR²⁶. In certain embodiments, at least one Z⁶ is independently —C(O)OR²⁶.

In certain embodiments, each Z⁷ is independently —C(O)R²⁶, —C(O)OR²⁶, or —C(O)N(R²⁷)(R²⁸). In certain embodiments, at least one Z⁷ is independently —C(O)R²⁶, —C(O)OR²⁶, or —C(O)N(R²⁷)(R²⁸).

In certain embodiments, each Z⁷ is independently —C(O)OR²⁶. In certain embodiments, at least one Z⁷ is independently —C(O)OR²⁶.

As disclosed above, any of the definitions for the variables provided (e.g., R¹, R², R³, R⁴, R, R⁶, R⁷, R⁸, R⁹, R¹⁰, R¹¹, R¹², R¹⁴, R¹⁵, R²¹, R²³, R²⁴, R²⁵, R²⁶, R²⁷, R²⁸, R^{a}, R^{b}, R^{c}, R^{d}, R^{e}, A R^{f}, R^{a1}, R^{2A}, R^{3A}, R^{3B}, X¹, X², X³, X⁴, X⁵, R^{x4}, R^{x5}, Z⁵, Z⁶, Z⁷, A, ring \textcircled{A}, Q, p, s, and q.) may be combined and grouped with other variables, whether or not specifically recited together.

In certain embodiments, a compound of any of the formulae described herein, or a pharmaceutically acceptable salt thereof, has the variables as described herein.

In certain embodiments, the compound is:

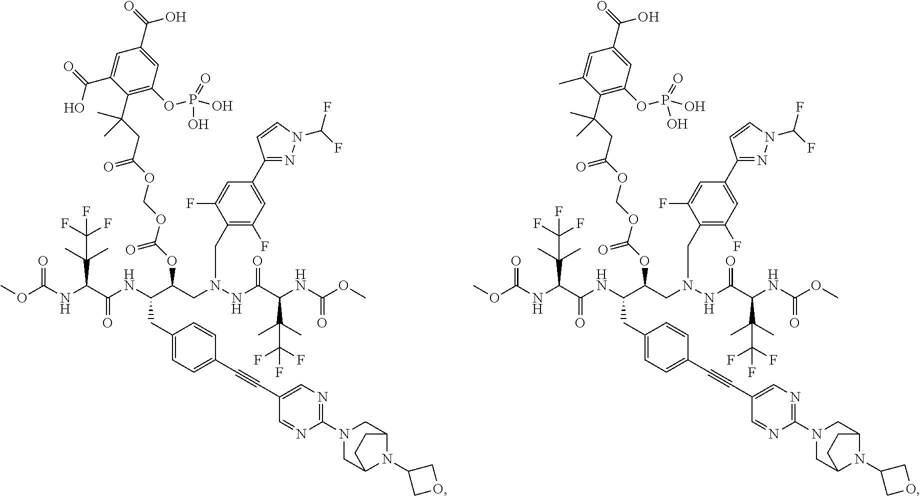 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(OP(=O)(O)O)cc(C(=O)O)cc1C(=O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(C)cc(C(=O)O)cc1OP(=O)(O)O)C(C)(C)C(F)(F)F]

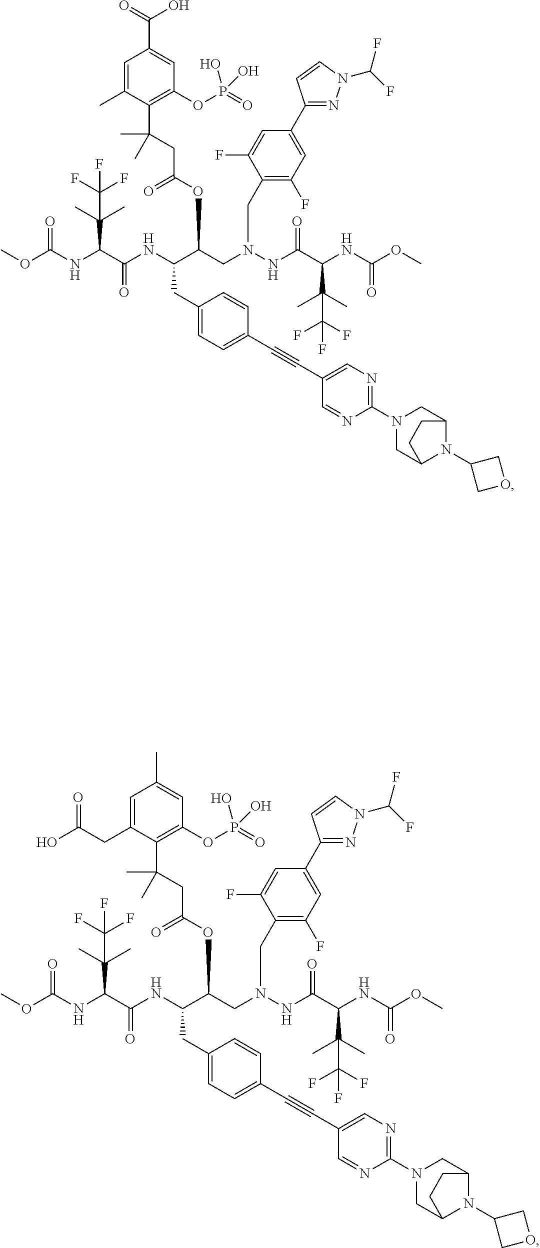 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(C)cc(C(=O)O)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F]

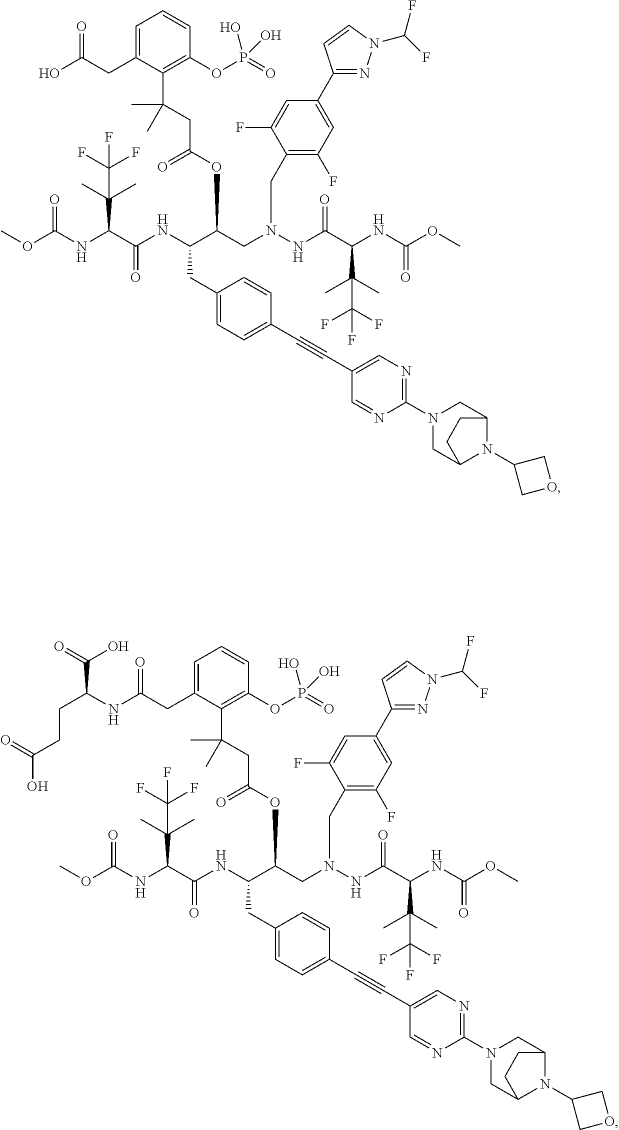 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cccc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cccc1OP(=O)(O)O)C(C)(C)C(F)(F)F]

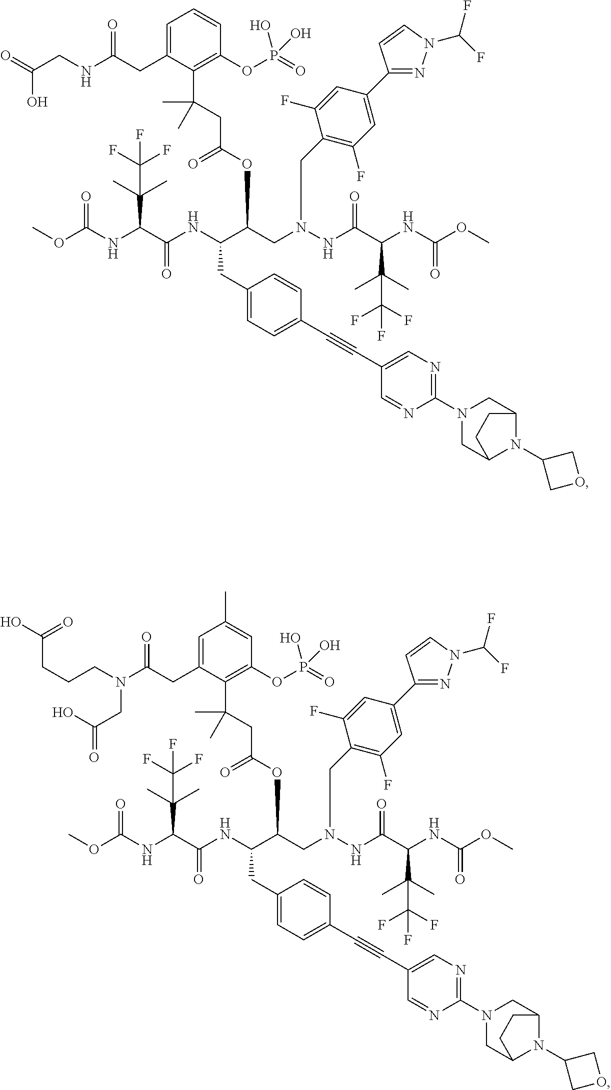 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)O)cccc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(CCCC(=O)O)CC(=O)O)cc(C)cc1OP(=O)(O)O)C(C)(C)C(F)(F)F]

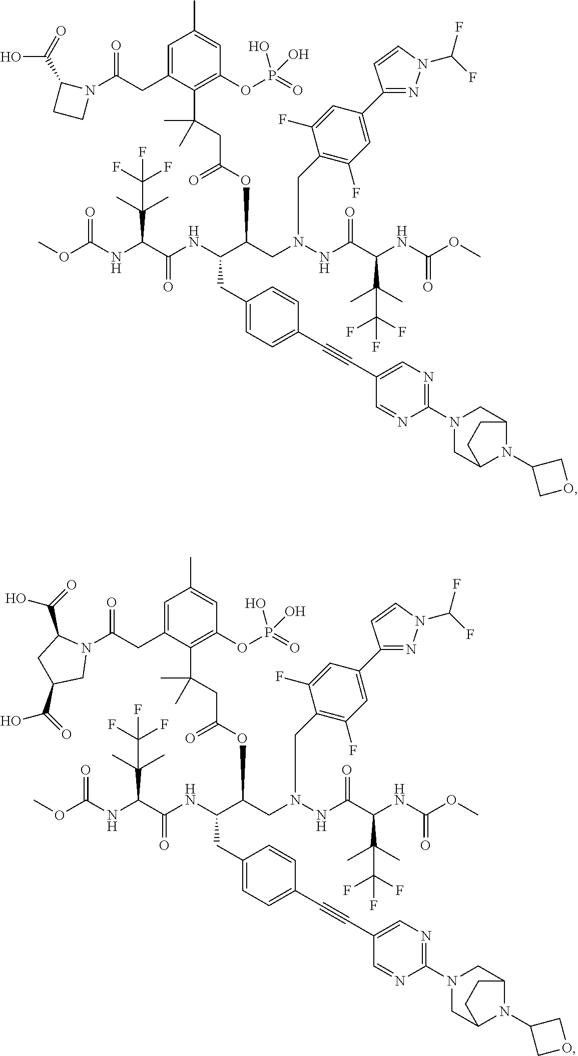 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC[C@@H]2C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2C[C@@H](C(=O)O)C[C@H]2C(=O)O)cc(C)cc1OP(=O)(O)O)C(C)(C)C(F)(F)F]

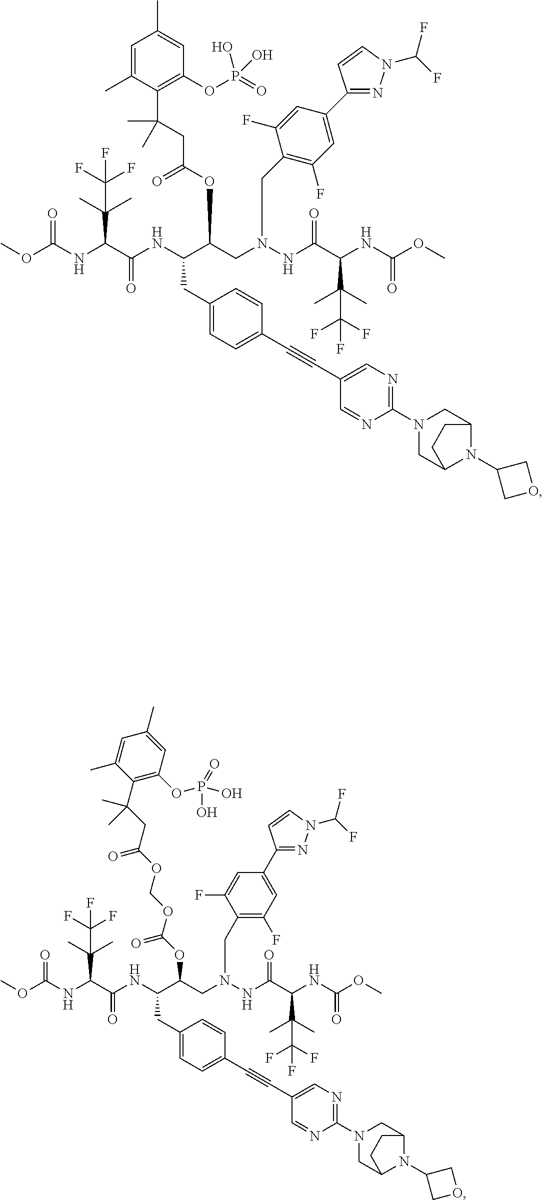 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(C)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(C)cc(C)cc1OP(=O)(O)O)C(C)(C)C(F)(F)F]

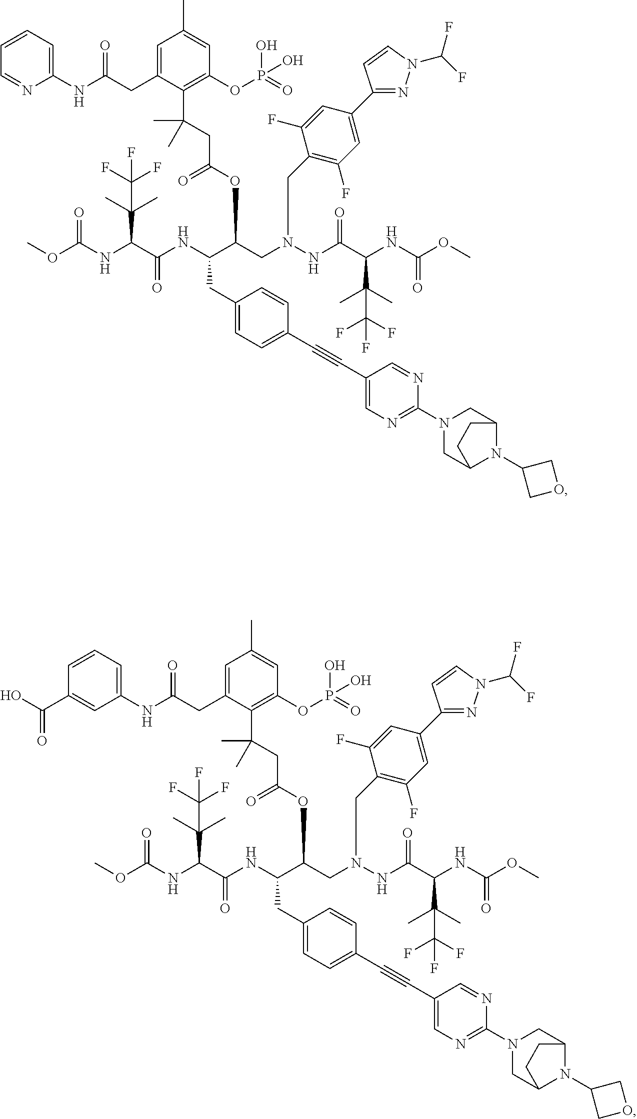 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccccn2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(C(=O)O)c2)cc(C)cc1OP(=O)(O)O)C(C)(C)C(F)(F)F]

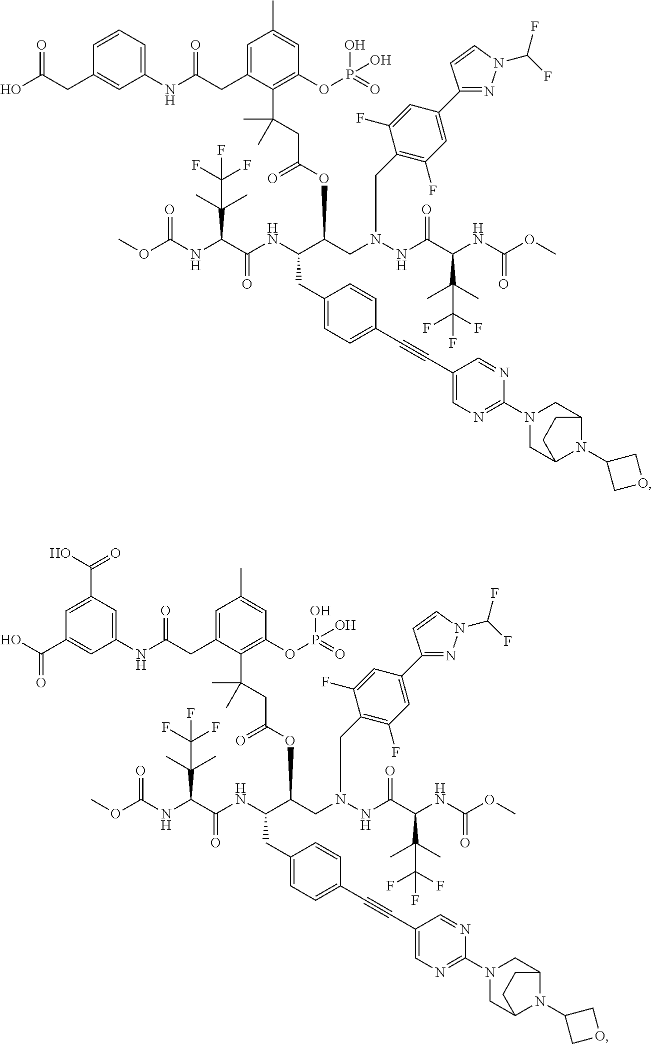 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(CC(=O)O)c2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)O)cc(C(=O)O)c2)cc(C)cc1OP(=O)(O)O)C(C)(C)C(F)(F)F]

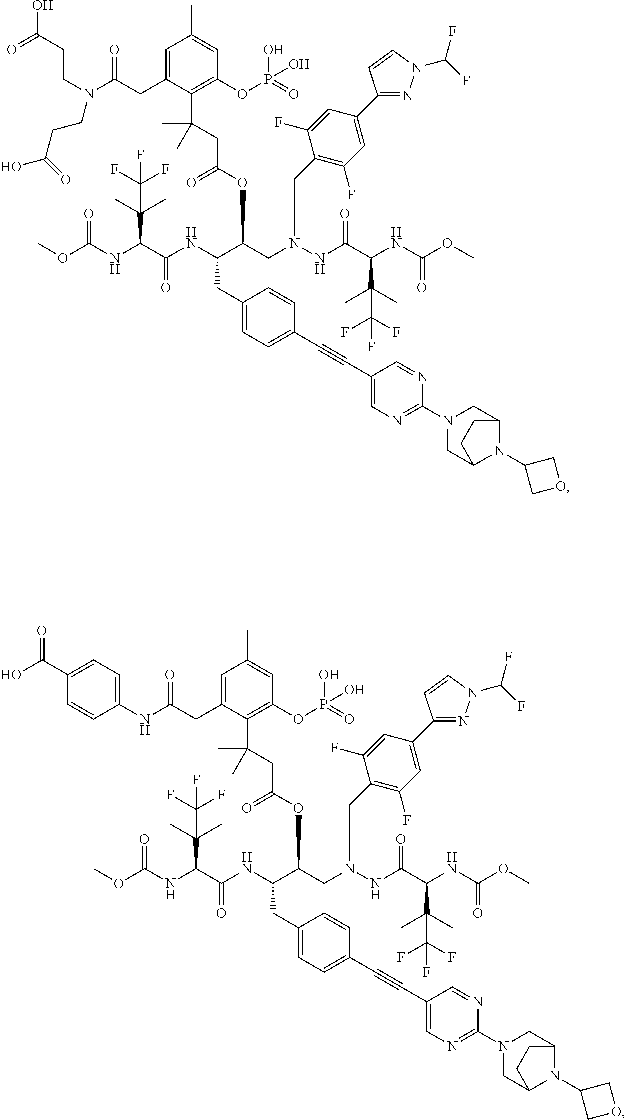 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(CCC(=O)O)CCC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccc(C(=O)O)cc2)cc(C)cc1OP(=O)(O)O)C(C)(C)C(F)(F)F]

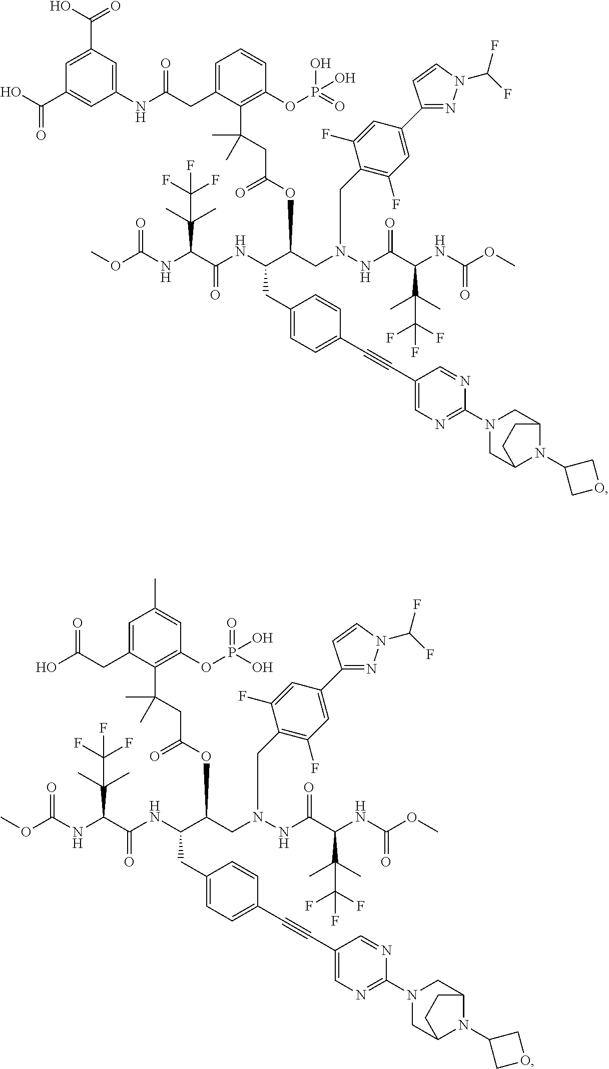 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)O)cc(C(=O)O)c2)cccc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F]

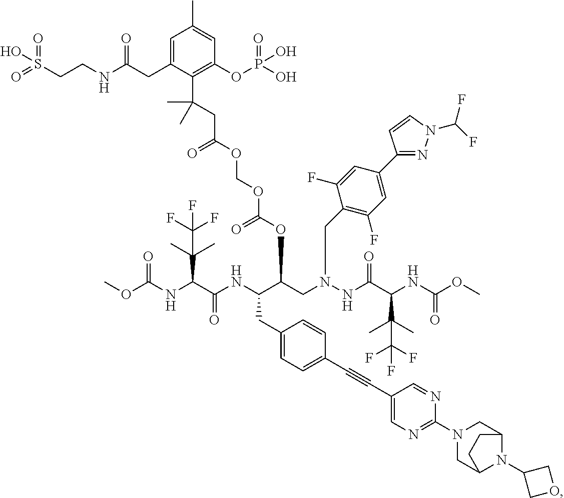 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCCS(=O)(=O)O)cc(C)cc1OP(=O)(O)O)C(C)(C)C(F)(F)F]

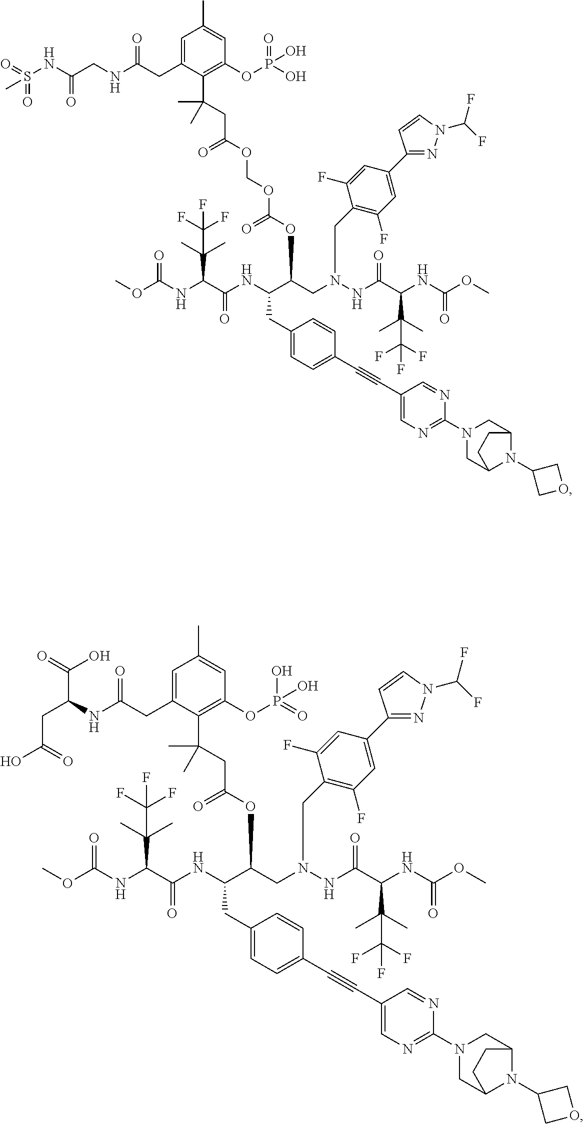 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCC(=O)NS(C)(=O)=O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CC(=O)O)C(=O)O)cc(C)cc1OP(=O)(O)O)C(C)(C)C(F)(F)F]

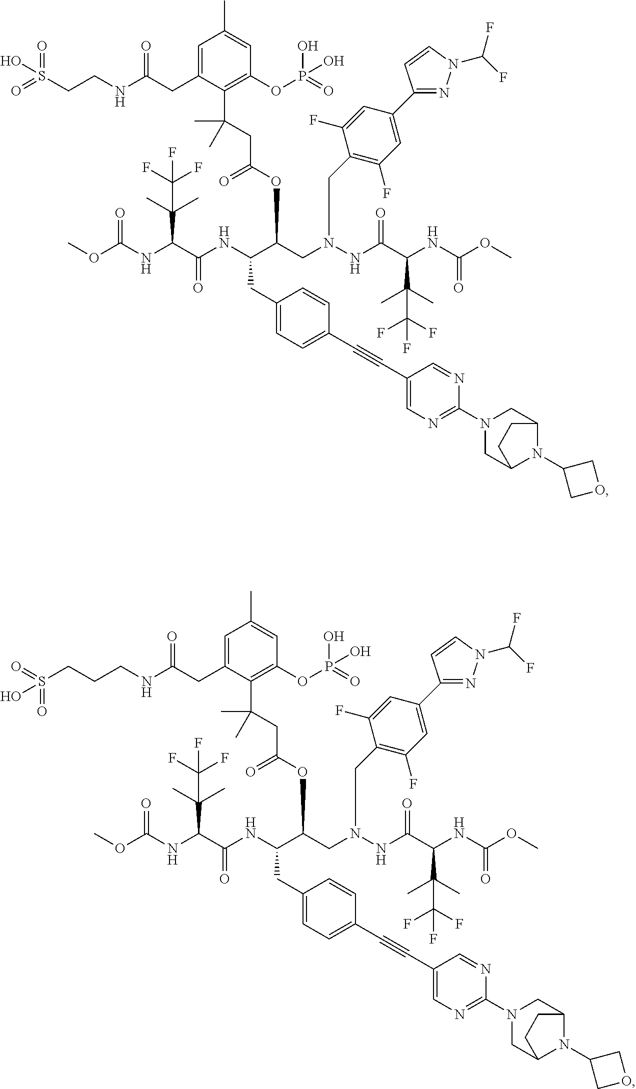 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCS(=O)(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCCS(=O)(=O)O)cc(C)cc1OP(=O)(O)O)C(C)(C)C(F)(F)F]

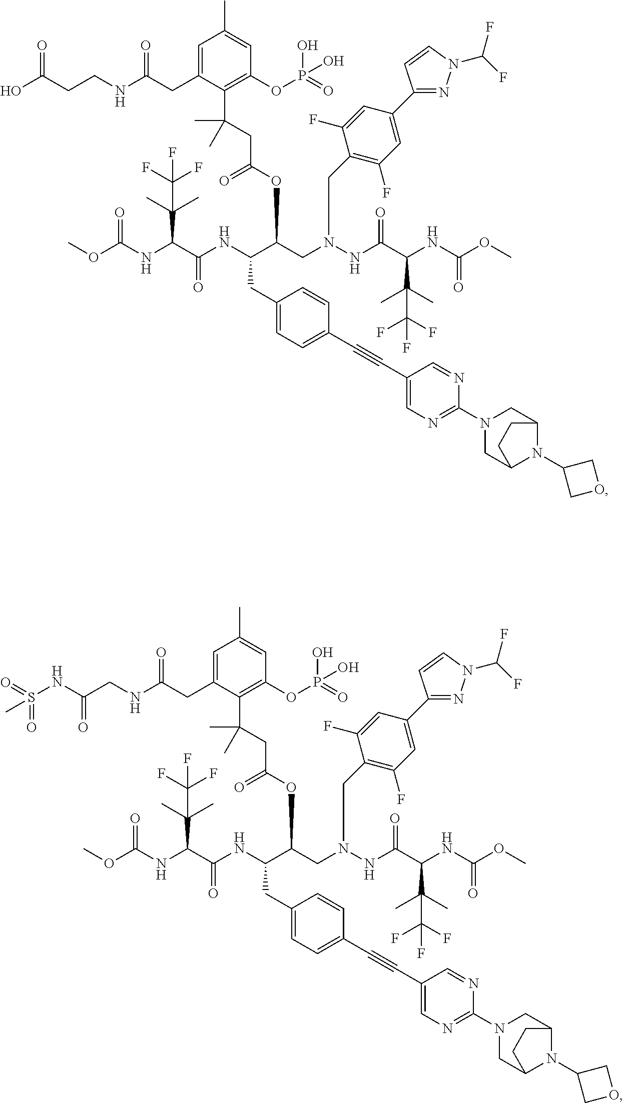 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)NS(C)(=O)=O)cc(C)cc1OP(=O)(O)O)C(C)(C)C(F)(F)F]

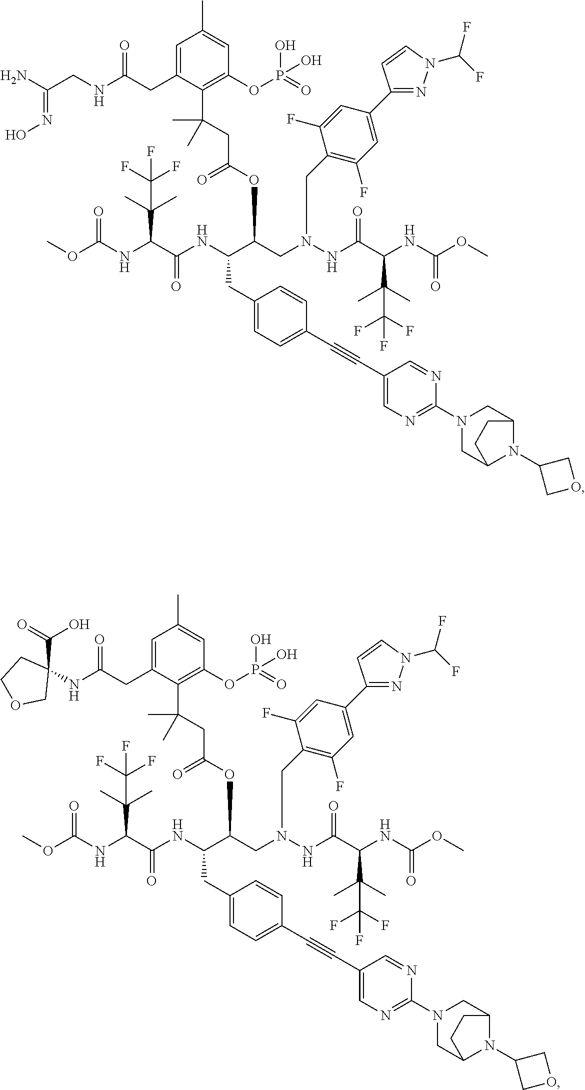 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC/C(N)=N/O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@]2(C(=O)O)CCOC2)cc(C)cc1OP(=O)(O)O)C(C)(C)C(F)(F)F]

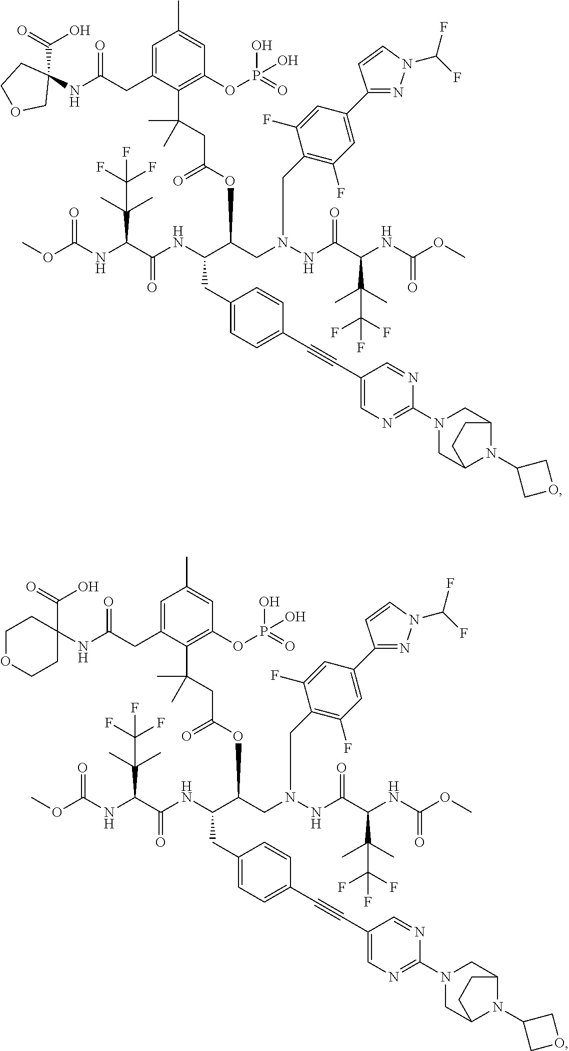 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@]2(C(=O)O)CCOC2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC2(C(=O)O)CCOCC2)cc(C)cc1OP(=O)(O)O)C(C)(C)C(F)(F)F]

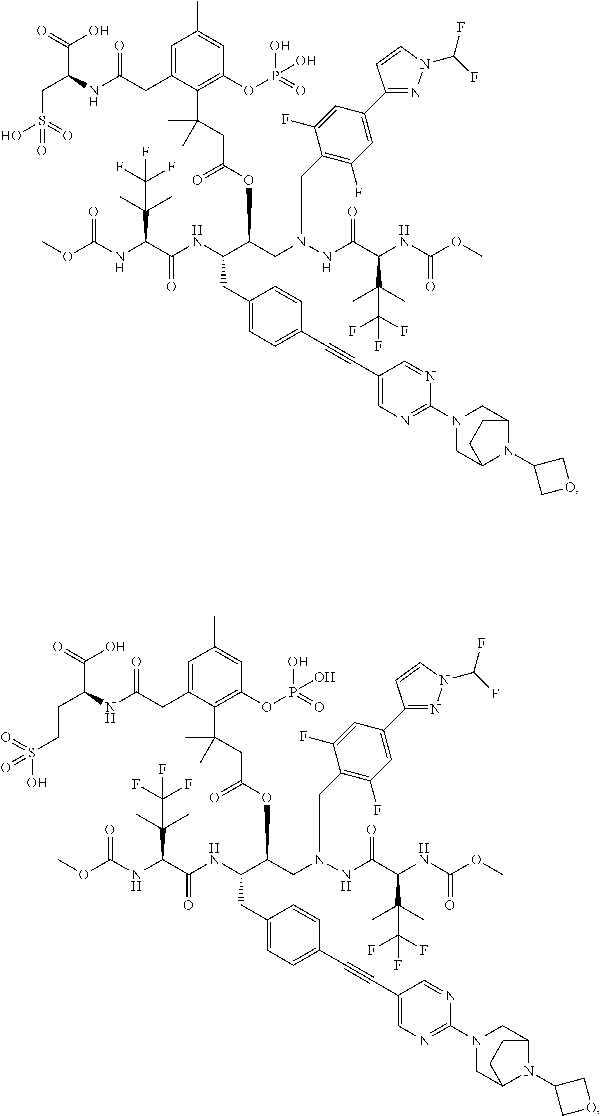 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCS(=O)(=O)O)C(=O)O)cc(C)cc1OP(=O)(O)O)C(C)(C)C(F)(F)F]

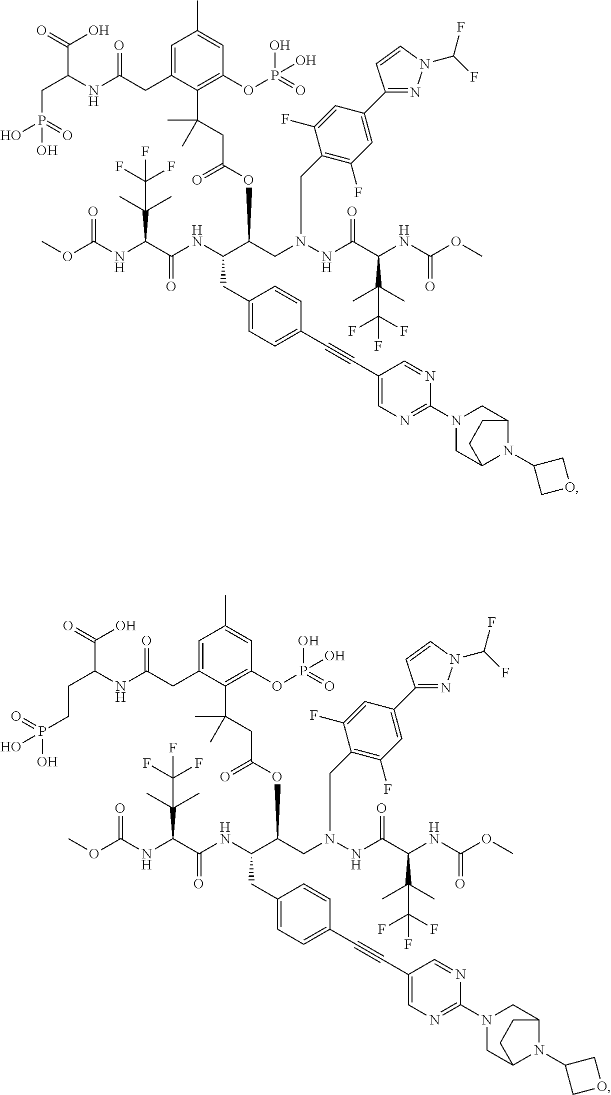 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CP(=O)(O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CCP(=O)(O)O)C(=O)O)cc(C)cc1OP(=O)(O)O)C(C)(C)C(F)(F)F]

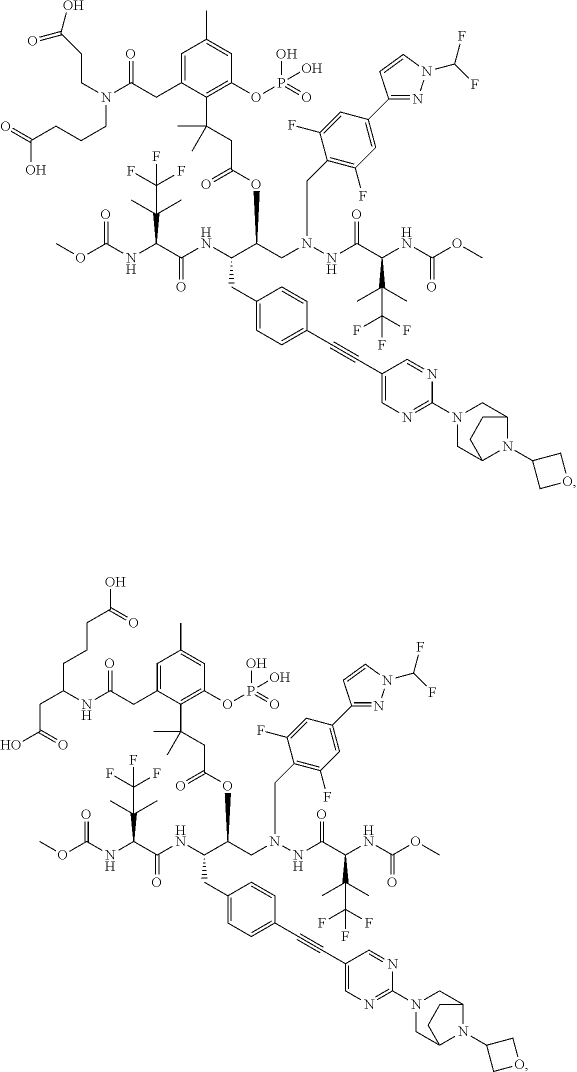 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(CCCC(=O)O)CCC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CCCC(=O)O)CC(=O)O)cc(C)cc1OP(=O)(O)O)C(C)(C)C(F)(F)F]

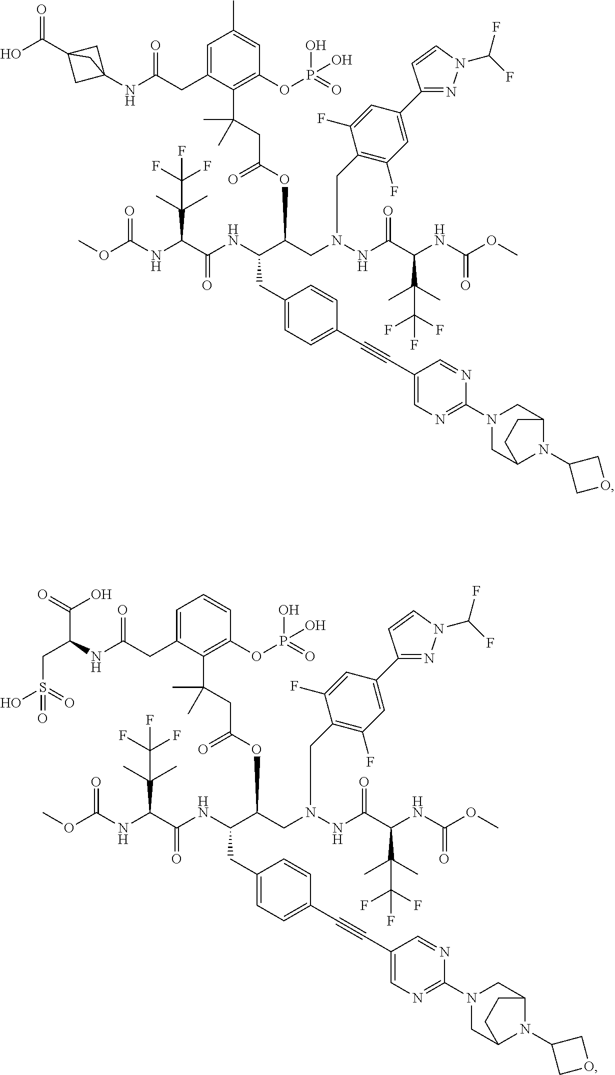 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC23CC(C(=O)O)(C2)C3)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cccc1OP(=O)(O)O)C(C)(C)C(F)(F)F]

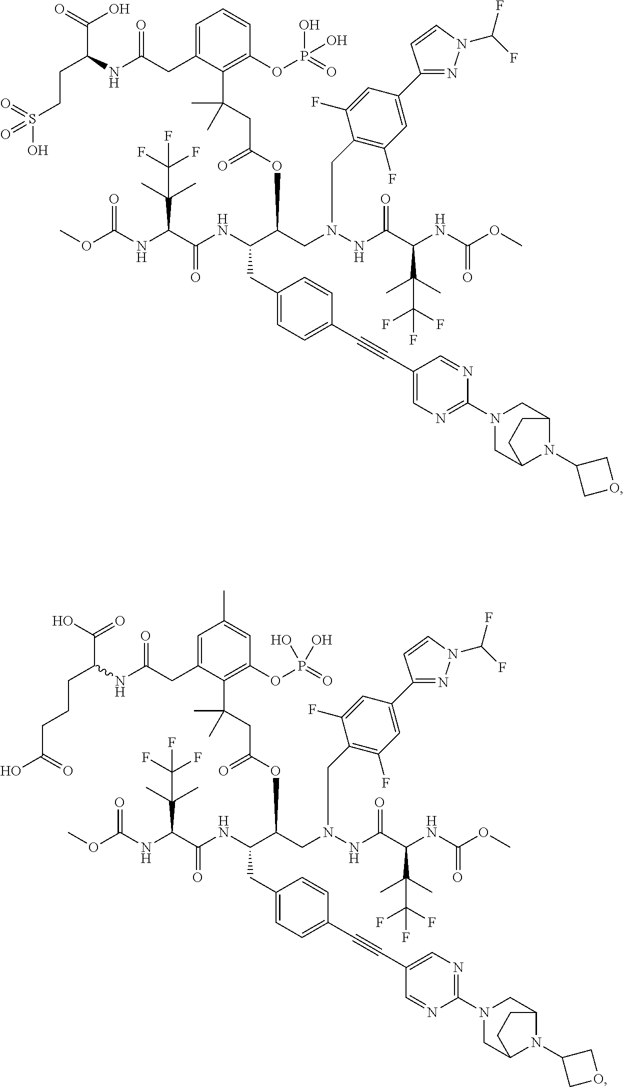 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCS(=O)(=O)O)C(=O)O)cccc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CCCC(=O)O)C(=O)O)cc(C)cc1OP(=O)(O)O)C(C)(C)C(F)(F)F]

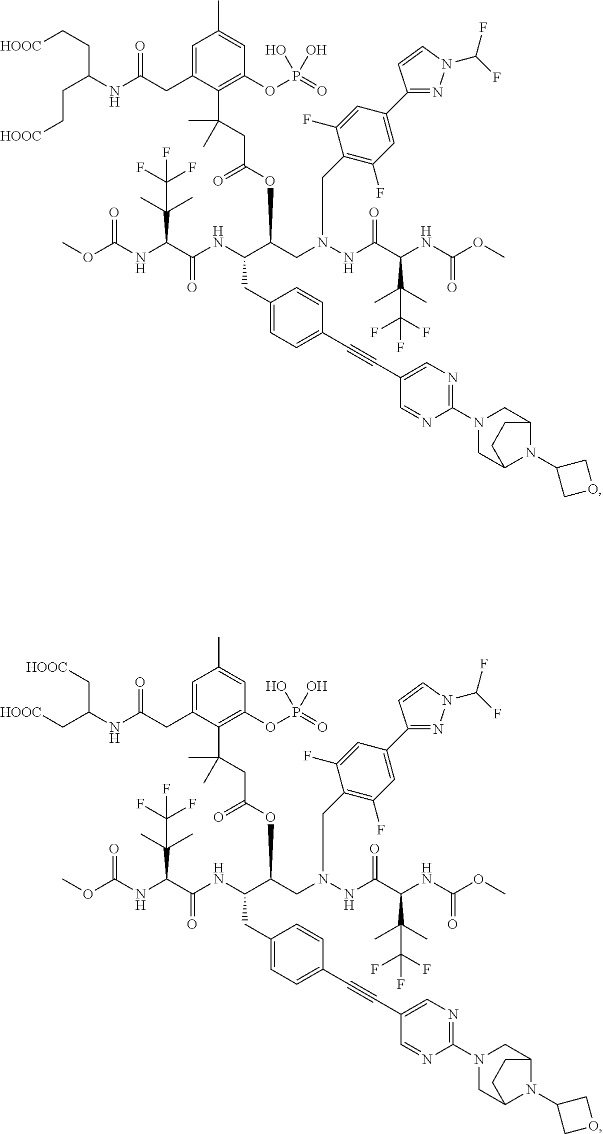 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CCC(=O)O)CCC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CC(=O)O)CC(=O)O)cc(C)cc1OP(=O)(O)O)C(C)(C)C(F)(F)F]

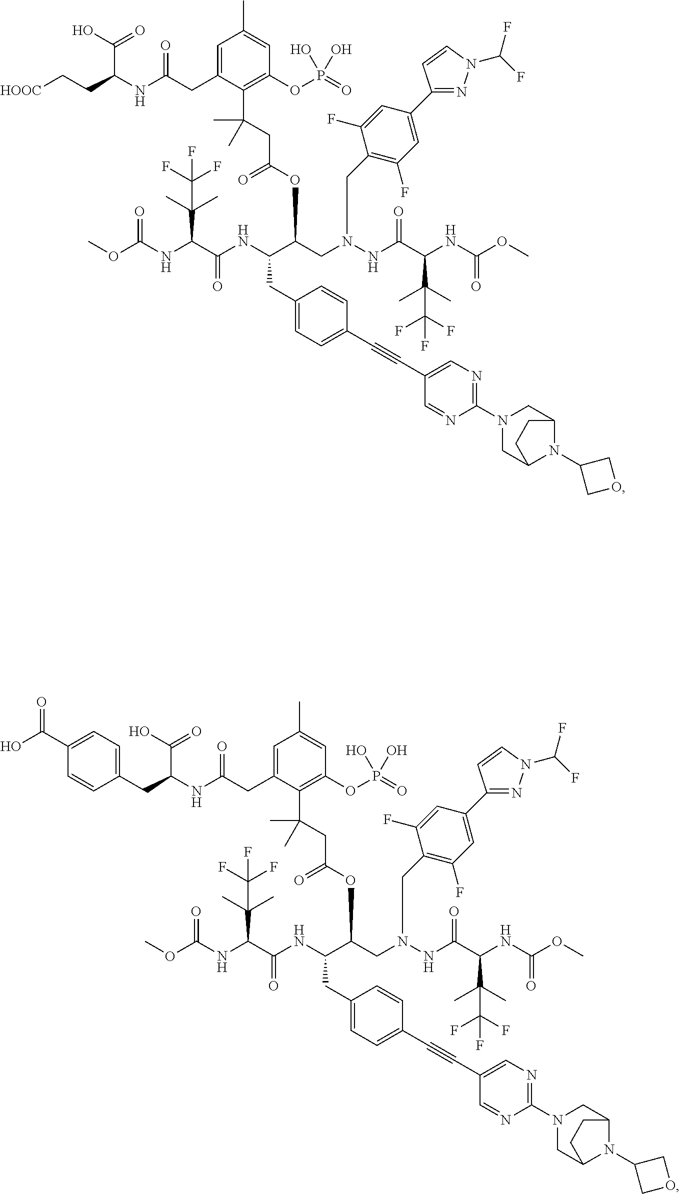 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](Cc2ccc(C(=O)O)cc2)C(=O)O)cc(C)cc1OP(=O)(O)O)C(C)(C)C(F)(F)F]

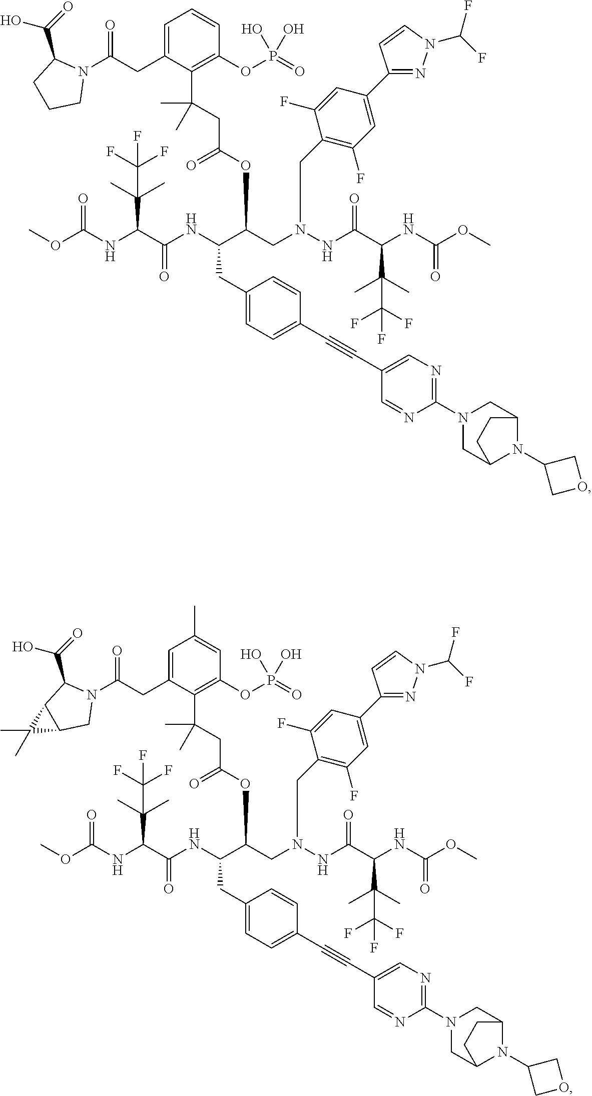 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@H]2C(=O)O)cccc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2C[C@H]3[C@@H]([C@H]2C(=O)O)C3(C)C)cc(C)cc1OP(=O)(O)O)C(C)(C)C(F)(F)F]

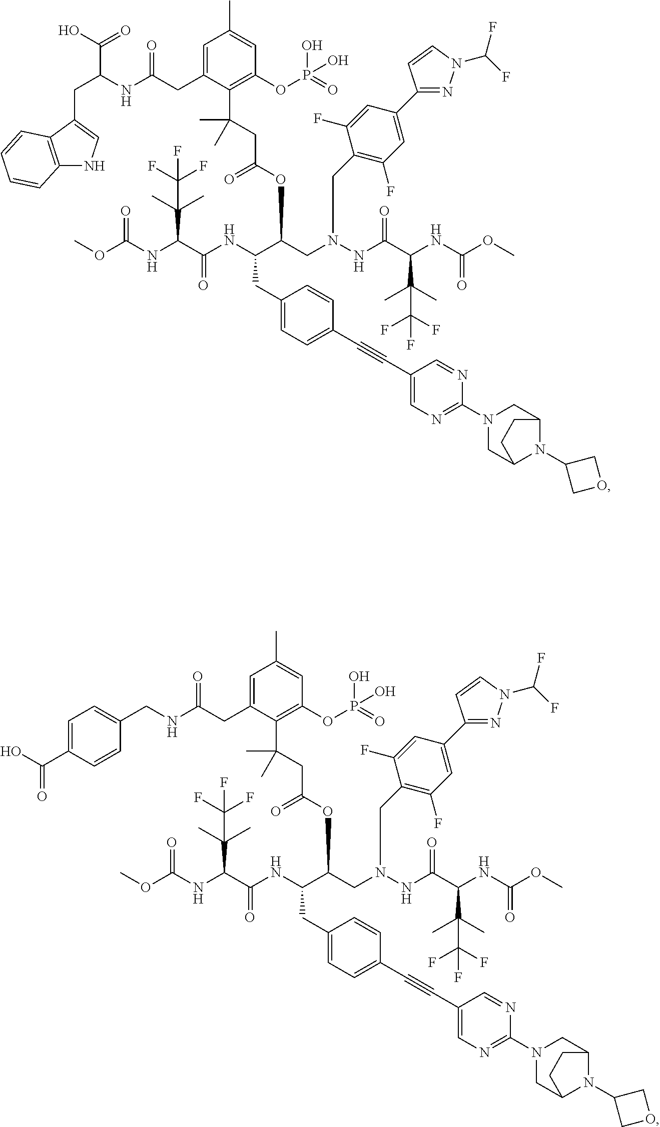 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(Cc2c[nH]c3ccccc23)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(C(=O)O)cc2)cc(C)cc1OP(=O)(O)O)C(C)(C)C(F)(F)F]

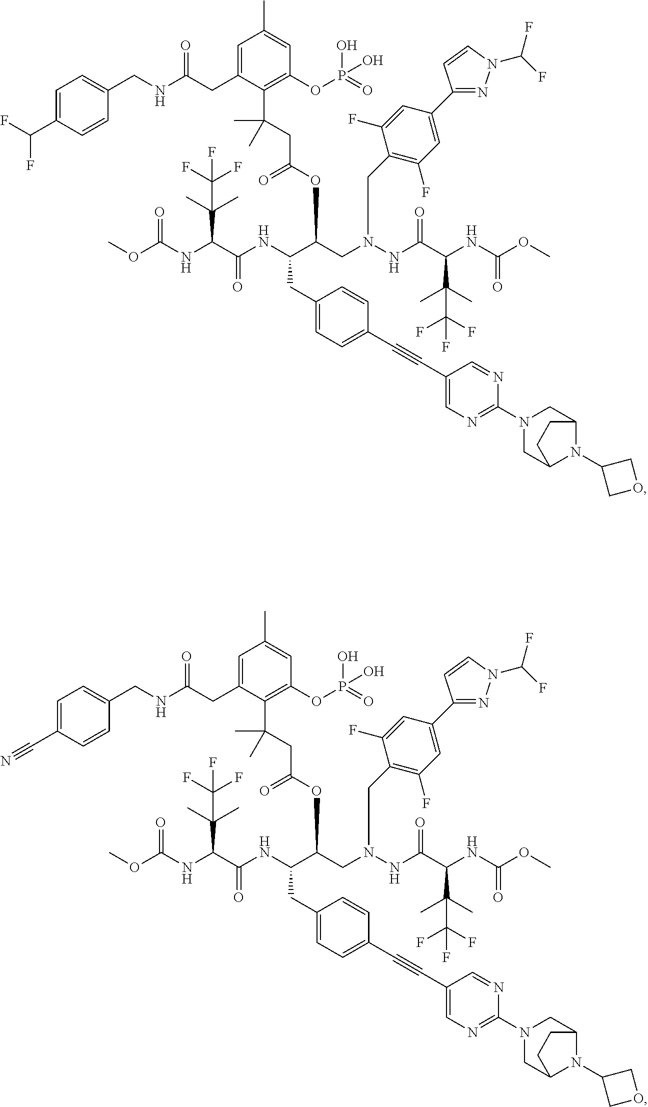 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(C(F)F)cc2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(C#N)cc2)cc(C)cc1OP(=O)(O)O)C(C)(C)C(F)(F)F]

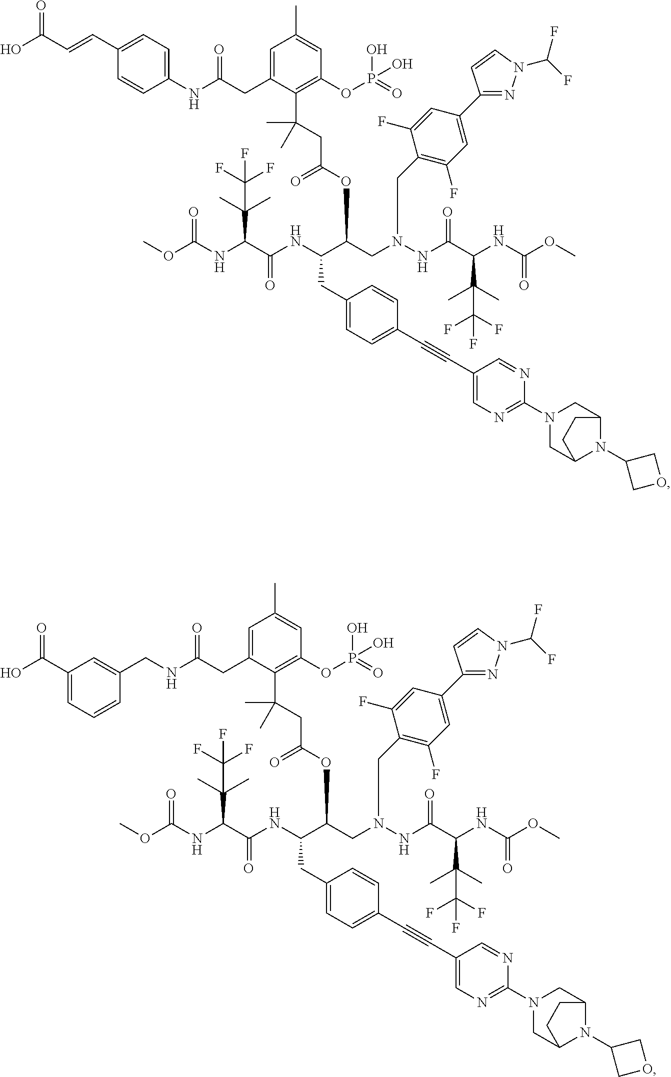 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccc(/C=C/C(=O)O)cc2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2cccc(C(=O)O)c2)cc(C)cc1OP(=O)(O)O)C(C)(C)C(F)(F)F]

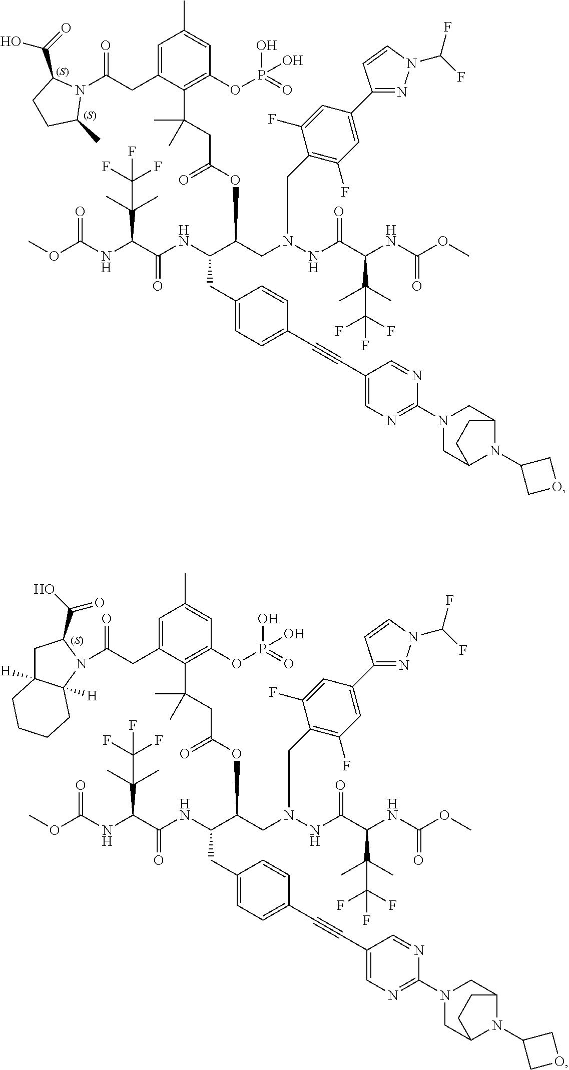

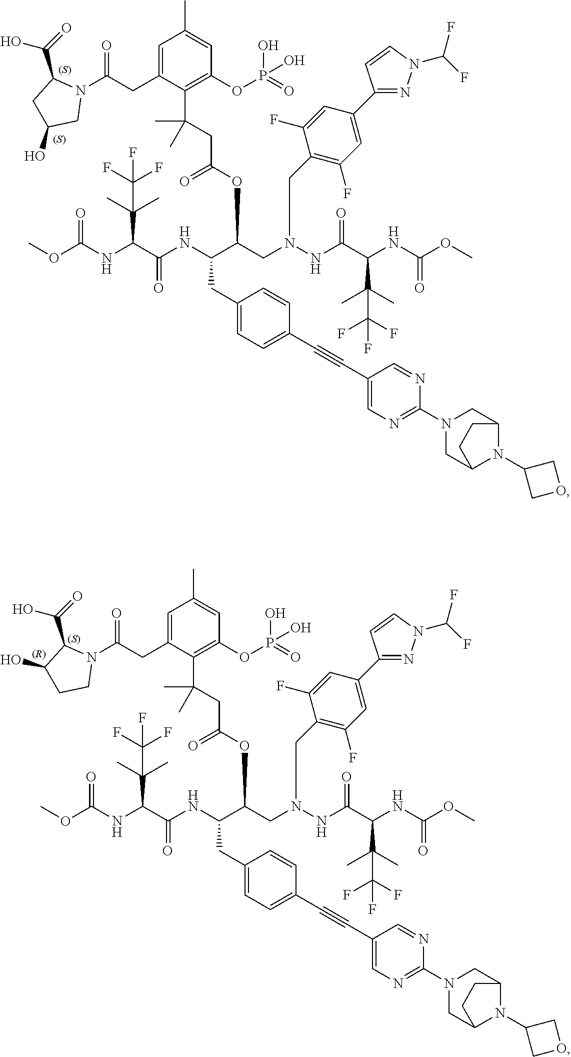

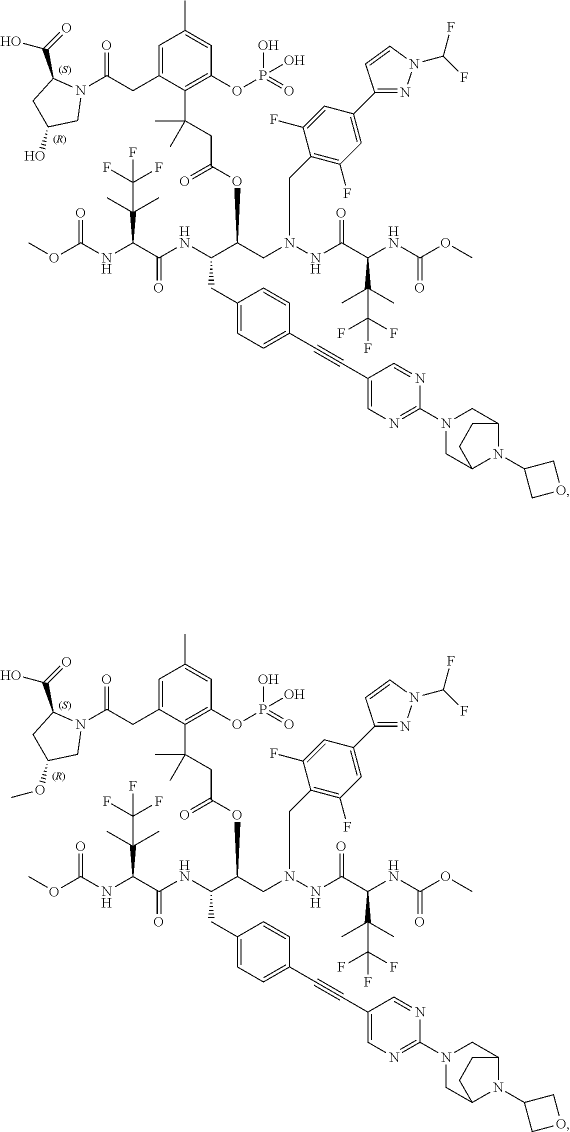

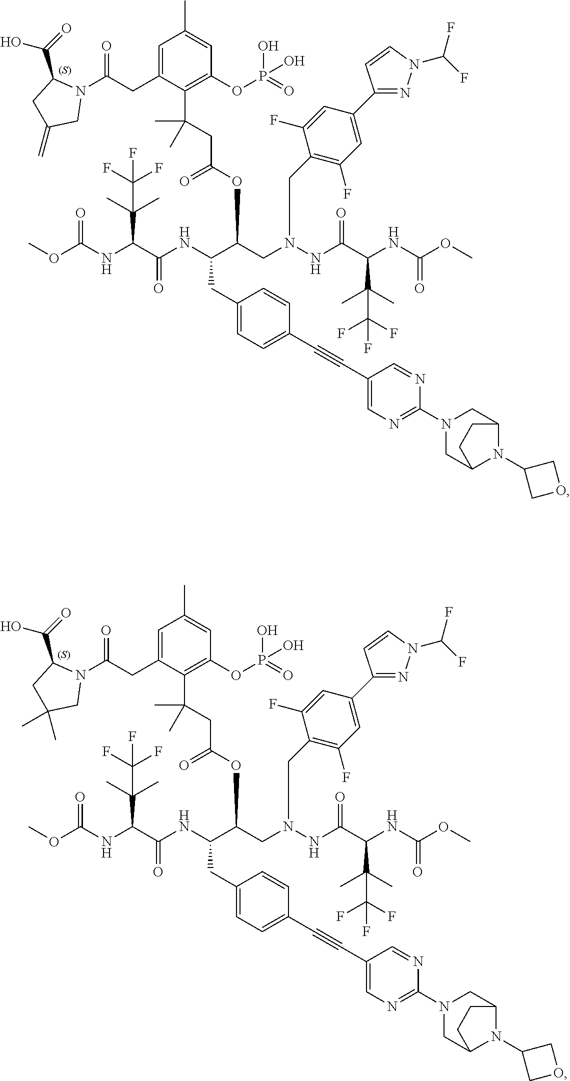

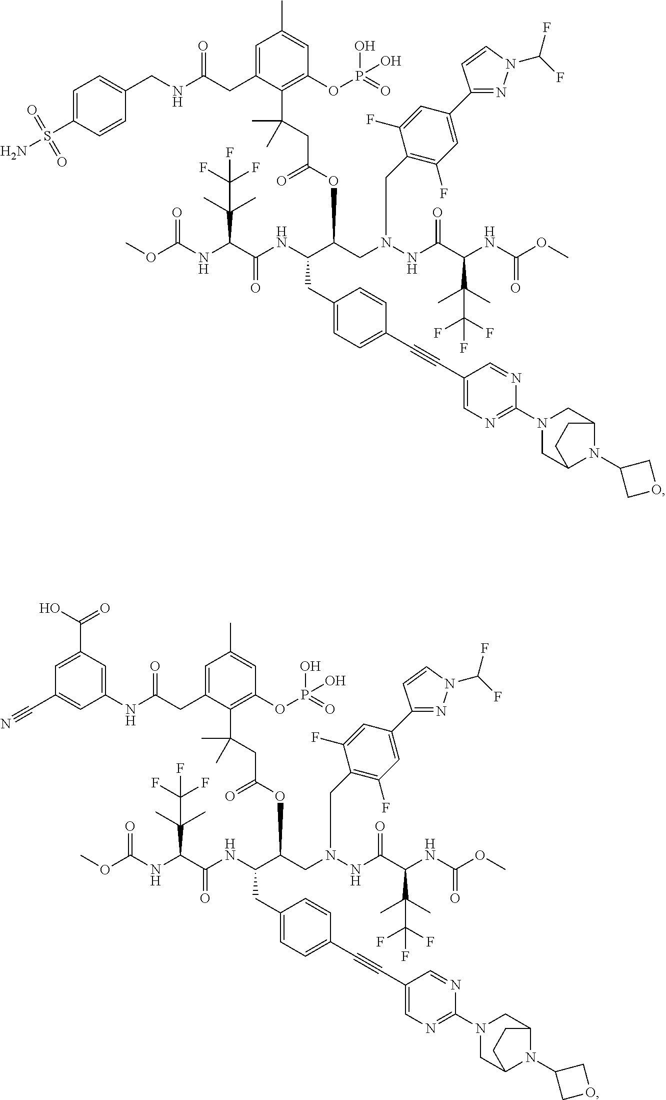 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(S(N)(=O)=O)cc2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C#N)cc(C(=O)O)c2)cc(C)cc1OP(=O)(O)O)C(C)(C)C(F)(F)F]

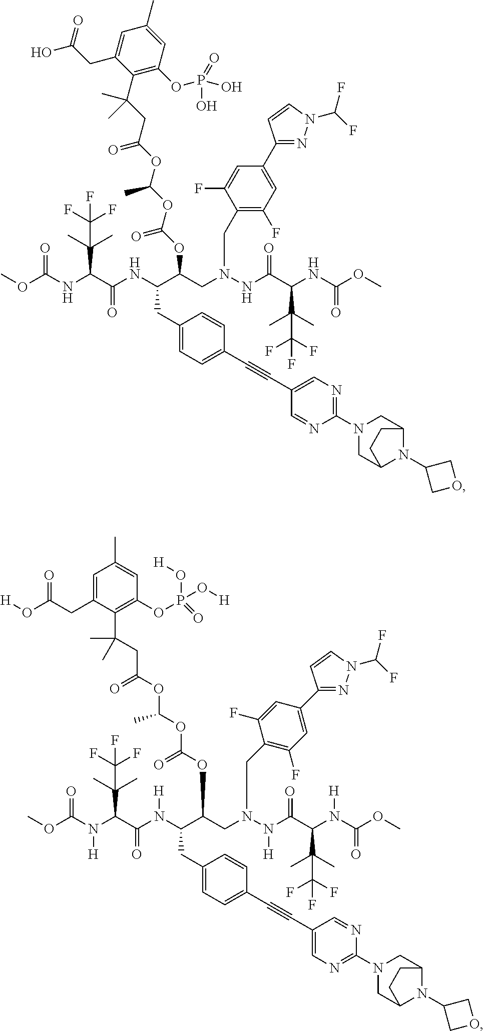 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)O[C@@H](C)OC(=O)CC(C)(C)c1c(CC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)O[C@H](C)OC(=O)CC(C)(C)c1c(CC(=O)O)cc(C)cc1OP(=O)(O)O)C(C)(C)C(F)(F)F]

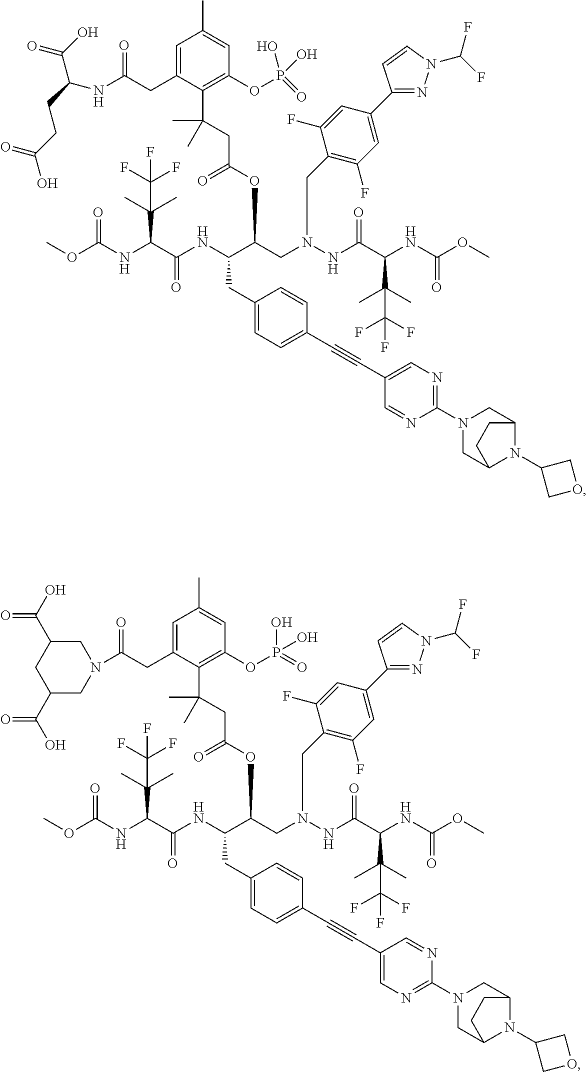 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC(C(=O)O)CC(C(=O)O)C2)cc(C)cc1OP(=O)(O)O)C(C)(C)C(F)(F)F]

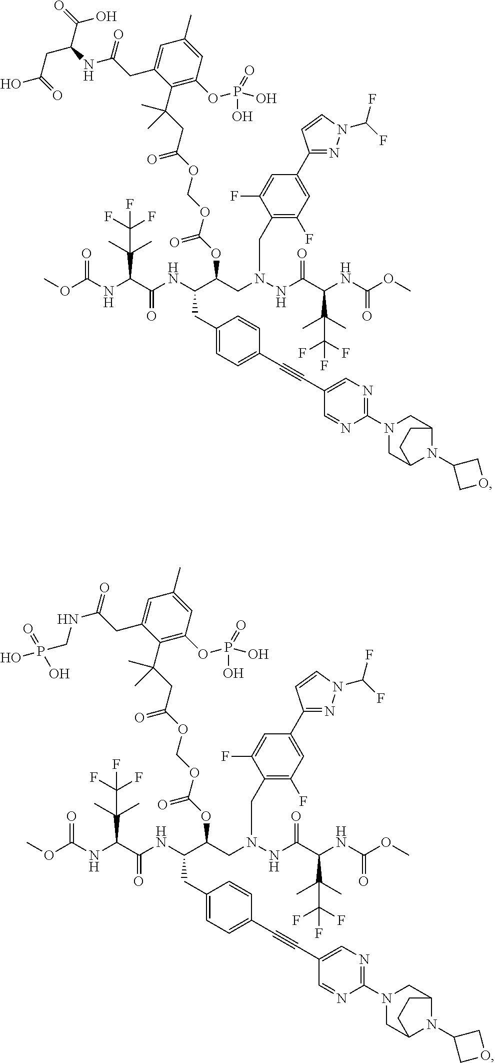 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CC(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCP(=O)(O)O)cc(C)cc1OP(=O)(O)O)C(C)(C)C(F)(F)F]

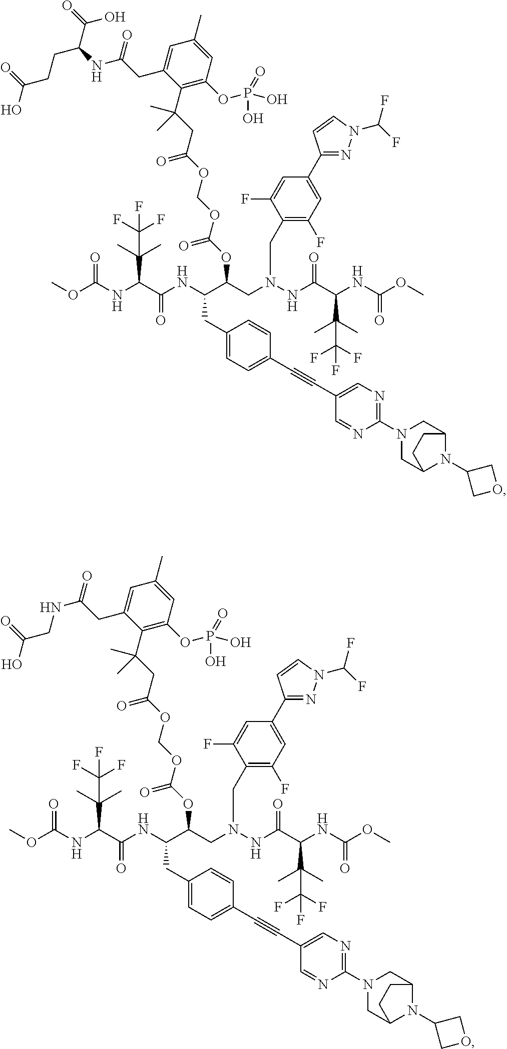 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CCC(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCC(=O)O)cc(C)cc1OP(=O)(O)O)C(C)(C)C(F)(F)F]

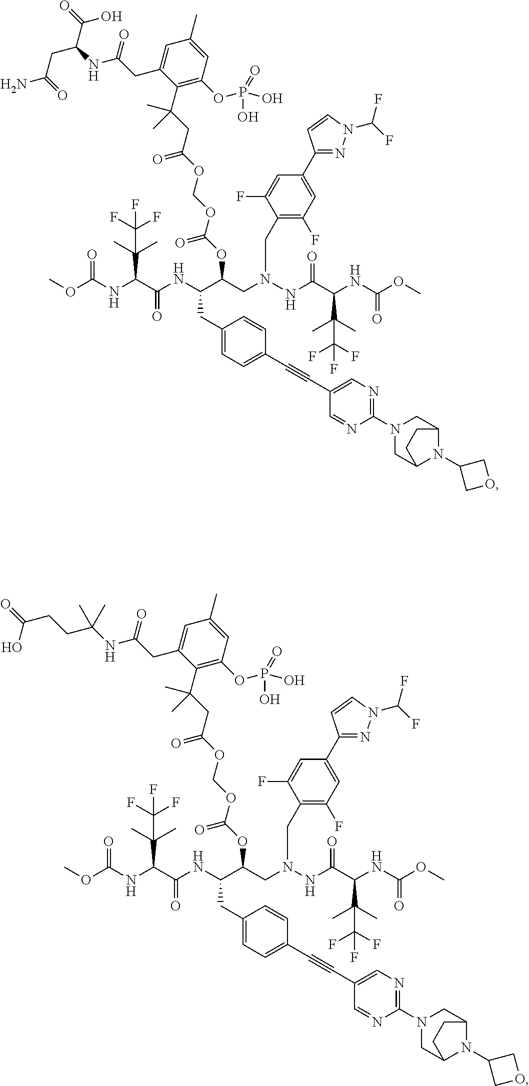 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CC(N)=O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NC(C)(C)CCC(=O)O)cc(C)cc1OP(=O)(O)O)C(C)(C)C(F)(F)F]

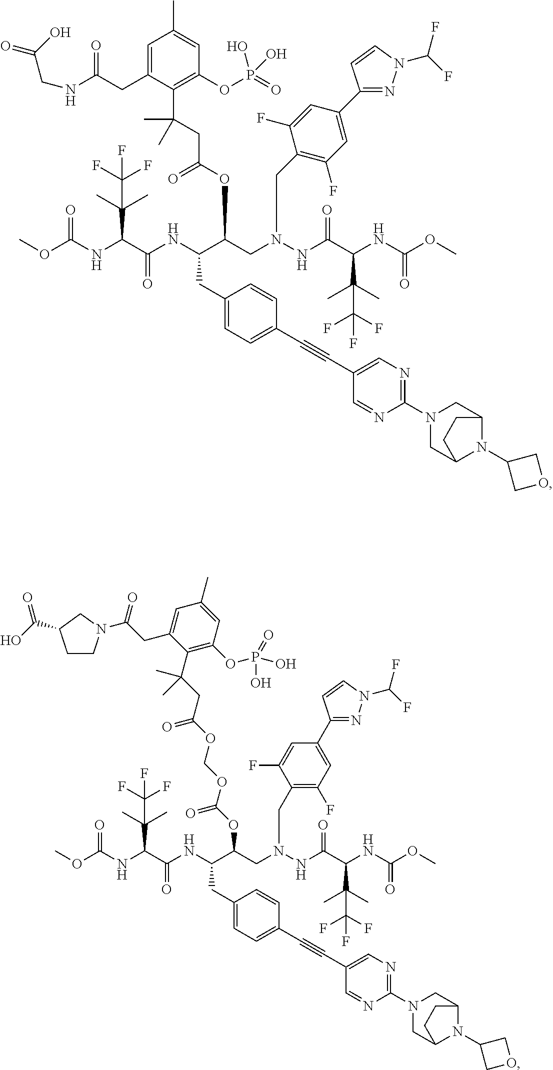 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CC[C@H](C(=O)O)C2)cc(C)cc1OP(=O)(O)O)C(C)(C)C(F)(F)F]

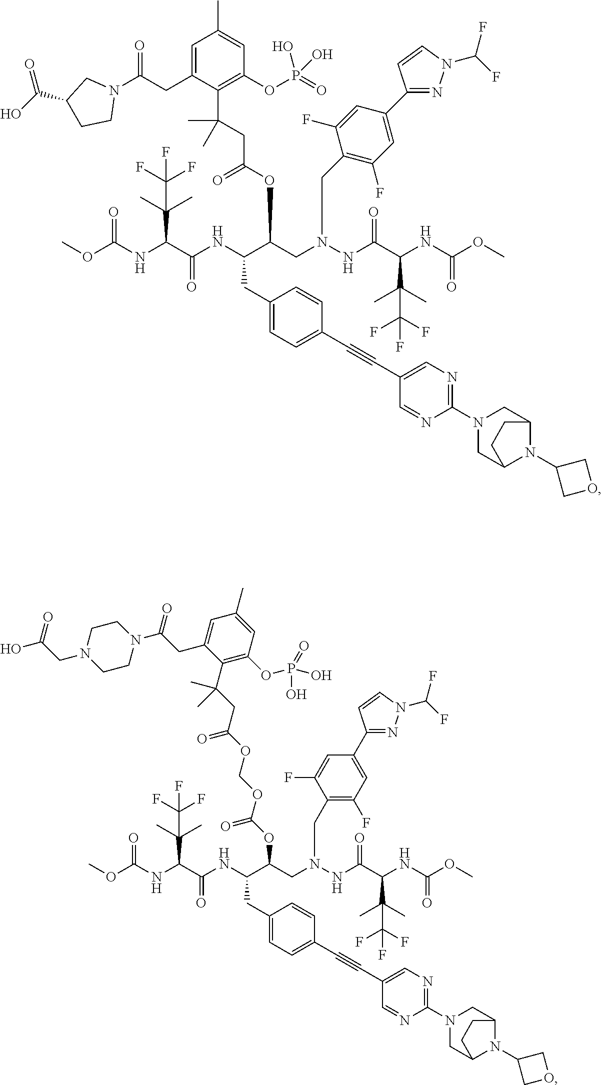 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC[C@H](C(=O)O)C2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(CC(=O)O)CC2)cc(C)cc1OP(=O)(O)O)C(C)(C)C(F)(F)F]

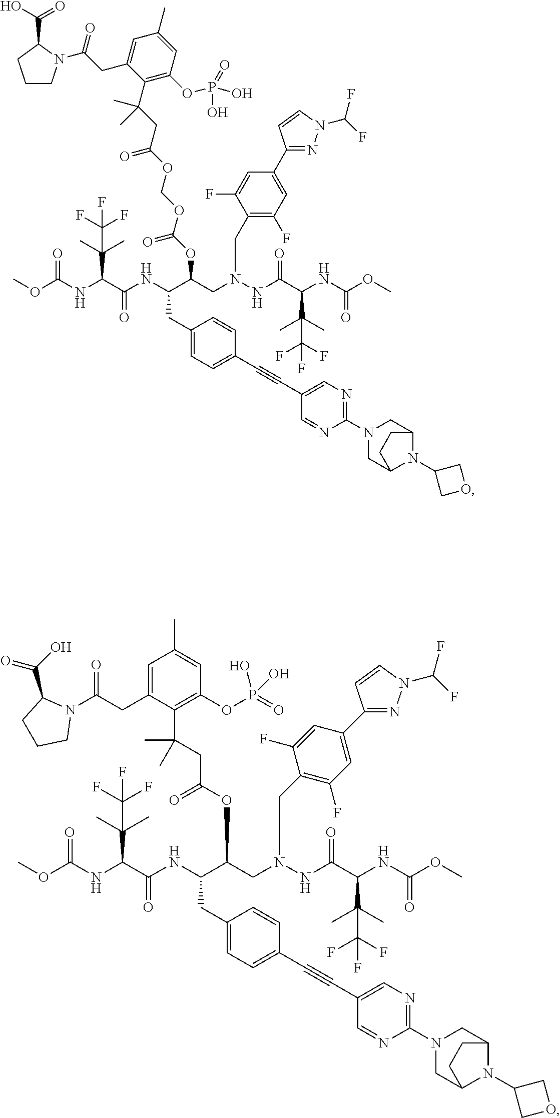 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCC[C@H]2C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@H]2C(=O)O)cc(C)cc1OP(=O)(O)O)C(C)(C)C(F)(F)F]

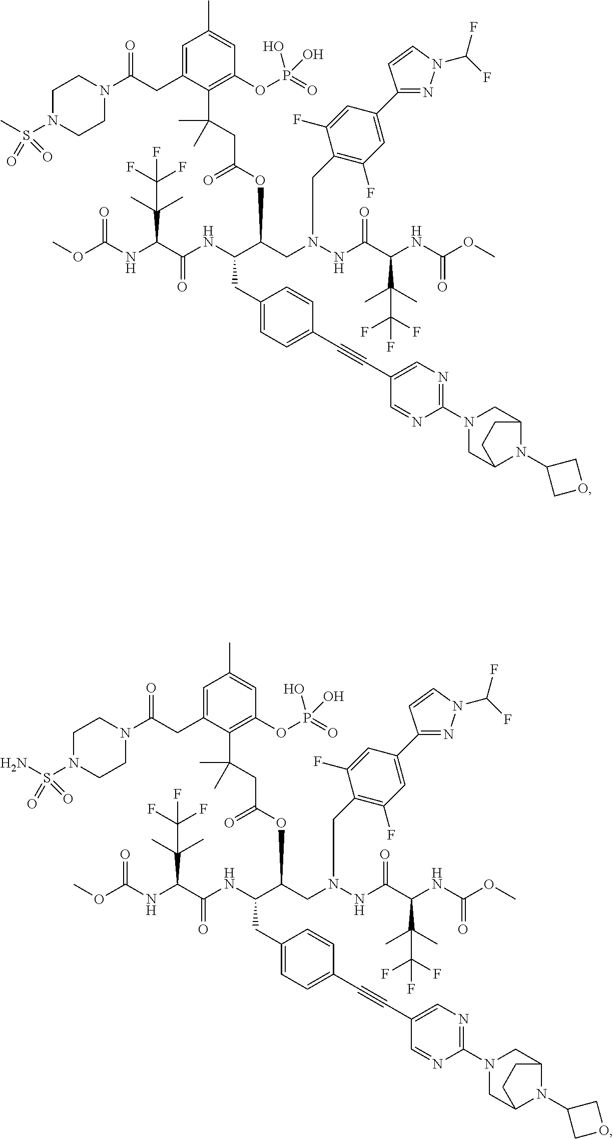 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCN(S(C)(=O)=O)CC2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCN(S(N)(=O)=O)CC2)cc(C)cc1OP(=O)(O)O)C(C)(C)C(F)(F)F]

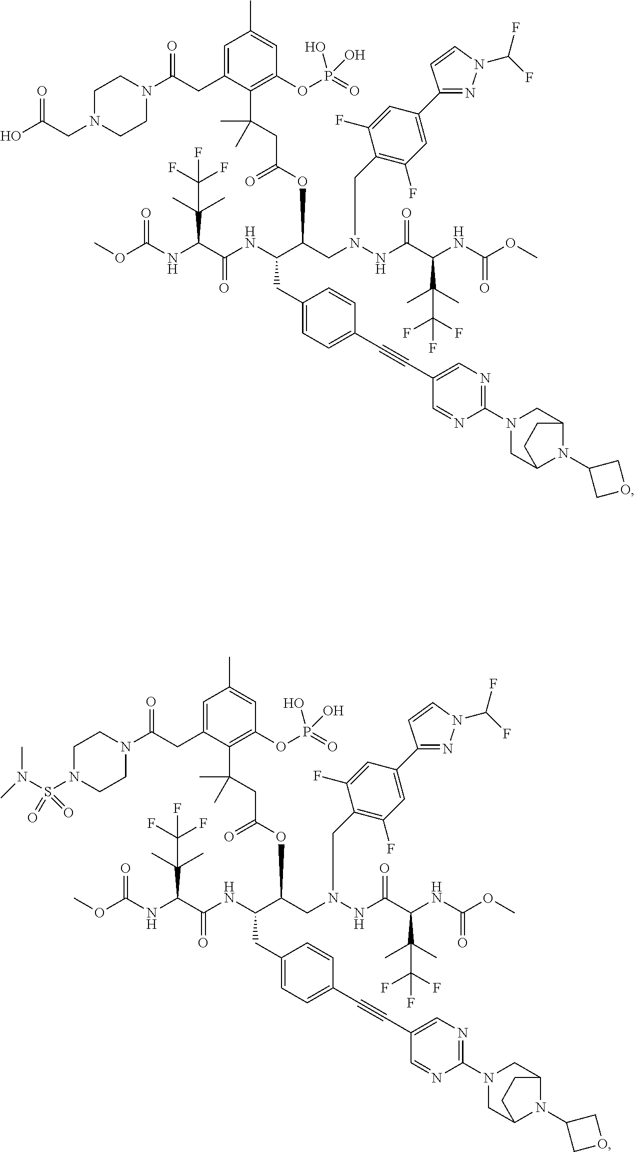 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCN(CC(=O)O)CC2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCN(S(=O)(=O)N(C)C)CC2)cc(C)cc1OP(=O)(O)O)C(C)(C)C(F)(F)F]

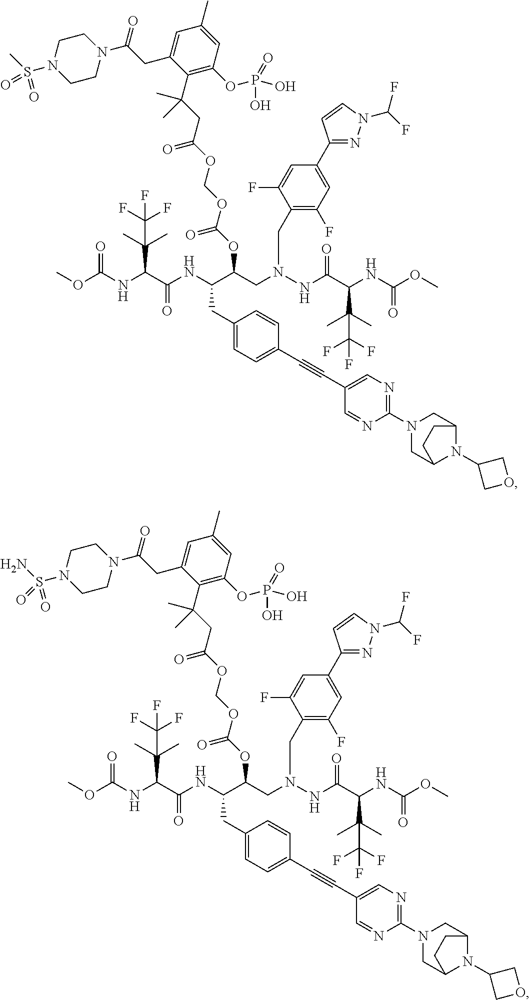 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(S(C)(=O)=O)CC2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(S(N)(=O)=O)CC2)cc(C)cc1OP(=O)(O)O)C(C)(C)C(F)(F)F]

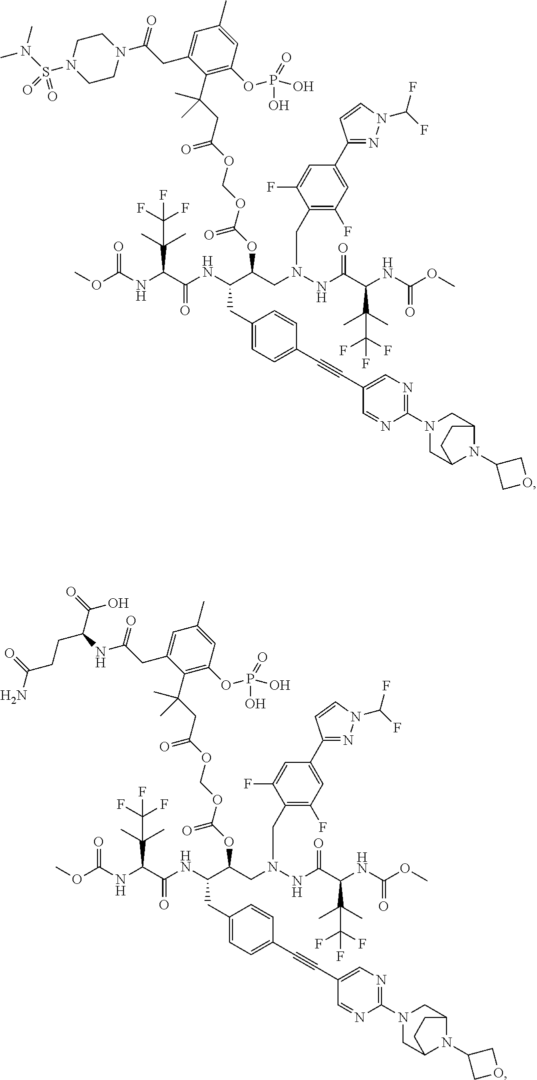 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(S(=O)(=O)N(C)C)CC2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CCC(N)=O)C(=O)O)cc(C)cc1OP(=O)(O)O)C(C)(C)C(F)(F)F]

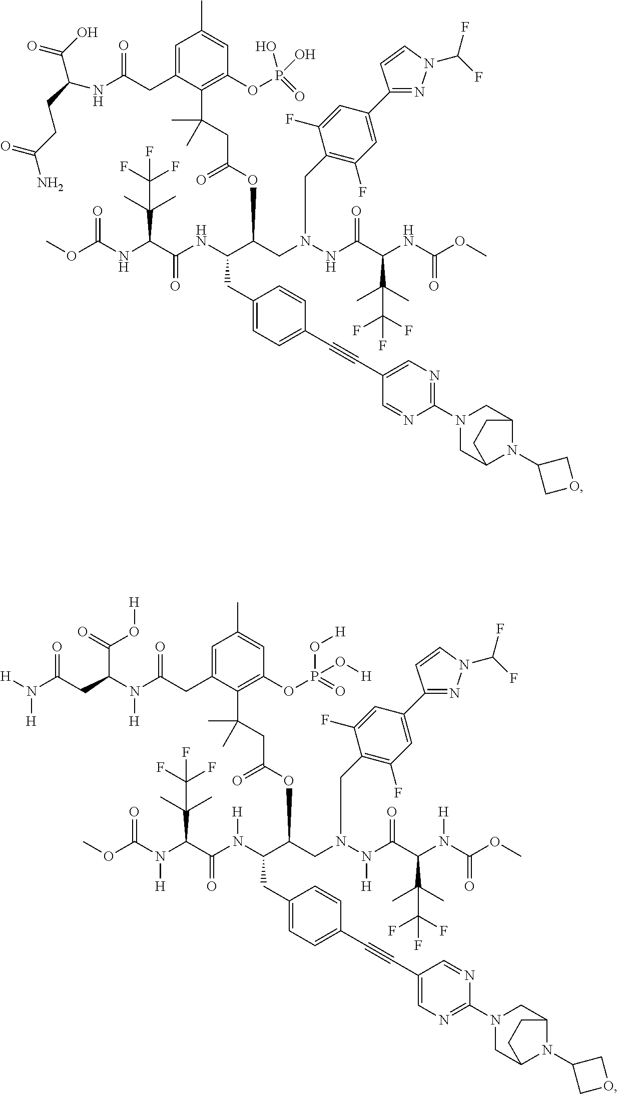 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(N)=O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CC(N)=O)C(=O)O)cc(C)cc1OP(=O)(O)O)C(C)(C)C(F)(F)F]

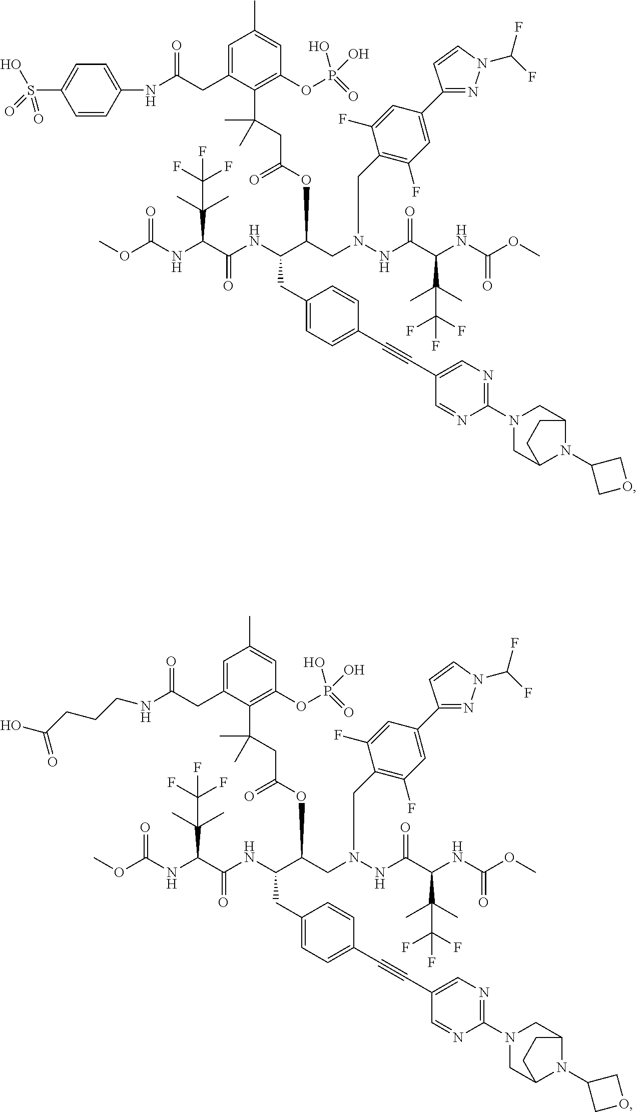 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccc(S(=O)(=O)O)cc2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCCC(=O)O)cc(C)cc1OP(=O)(O)O)C(C)(C)C(F)(F)F]

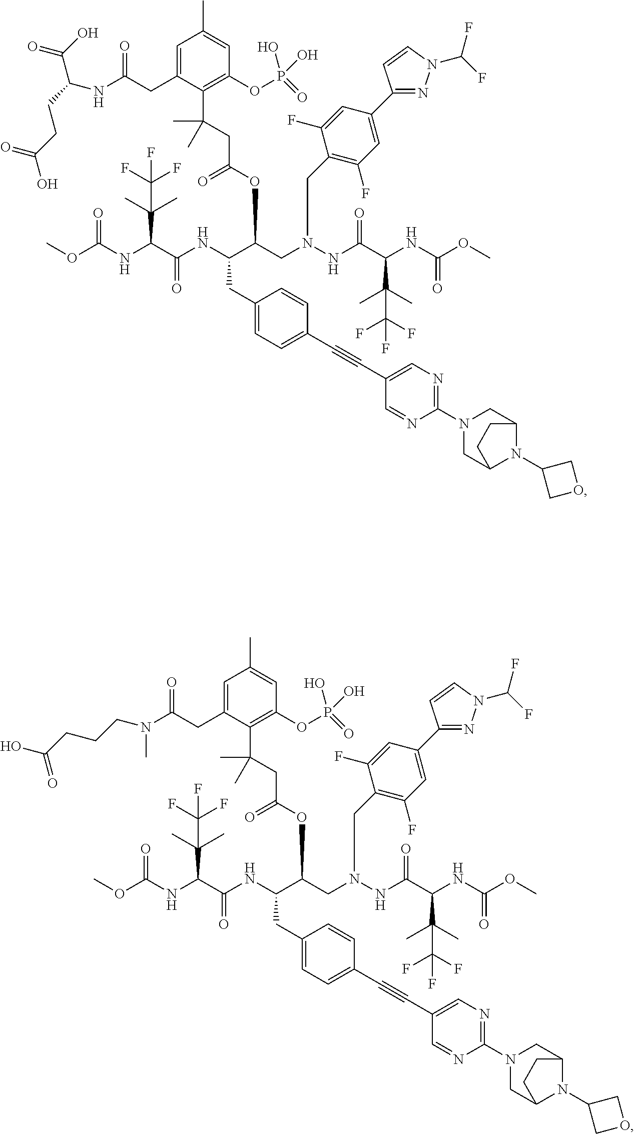 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H](CCC(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(C)CCCC(=O)O)cc(C)cc1OP(=O)(O)O)C(C)(C)C(F)(F)F]

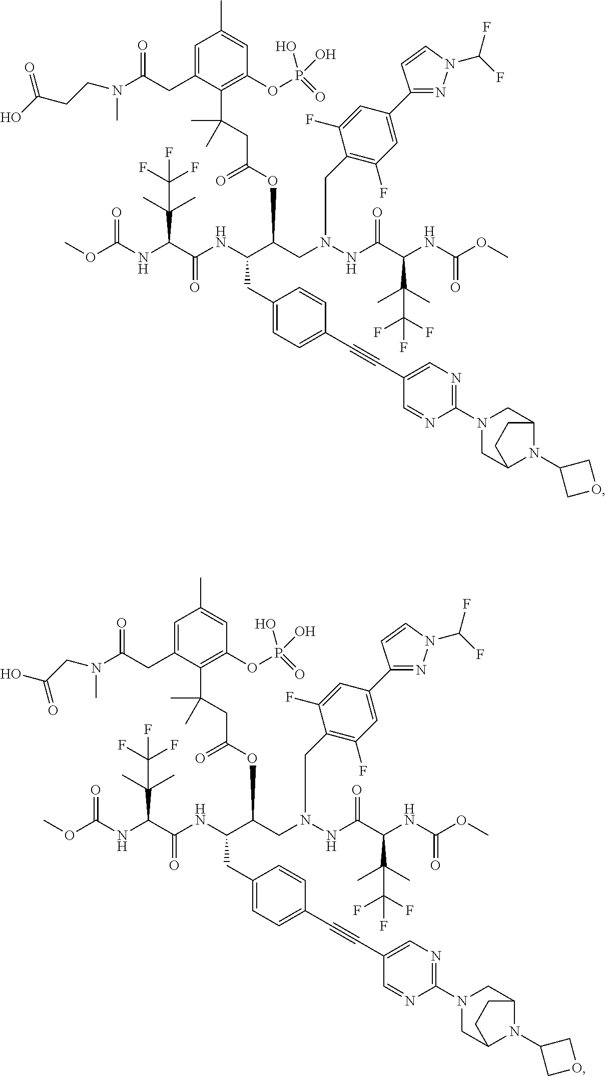 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(C)CCC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(C)CC(=O)O)cc(C)cc1OP(=O)(O)O)C(C)(C)C(F)(F)F]

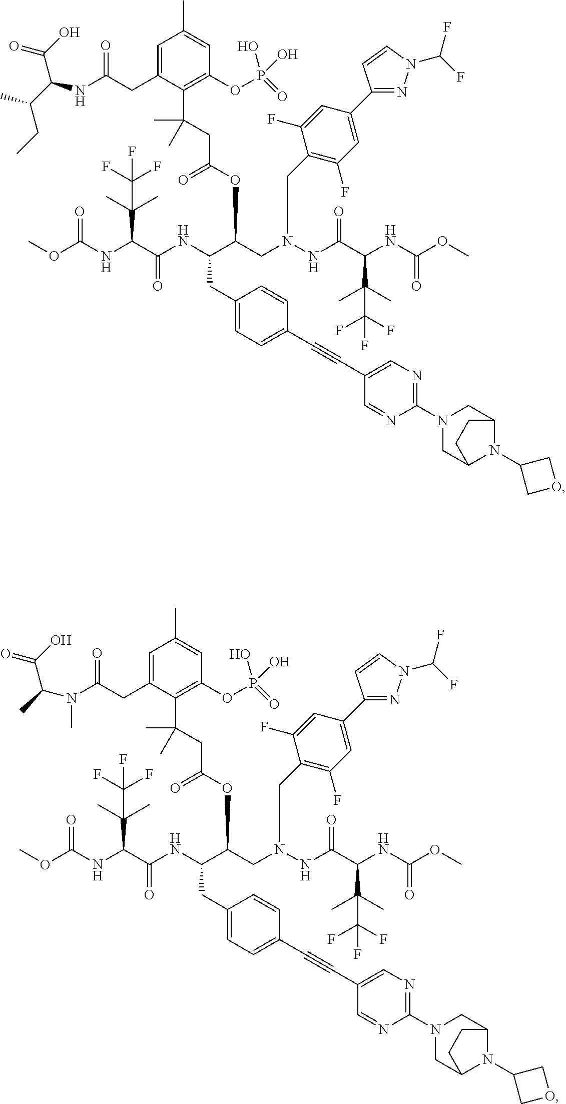 [SMILES: CC[C@H](C)[C@H](NC(=O)Cc1cc(C)cc(OP(=O)(O)O)c1C(C)(C)CC(=O)O[C@@H](CN(Cc1c(F)cc(-c2ccn(C(F)F)n2)cc1F)NC(=O)[C@@H](NC(=O)OC)C(C)(C)C(F)(F)F)[C@H](Cc1ccc(C#Cc2cnc(N3CC4CCC(C3)N4C3COC3)nc2)cc1)NC(=O)[C@@H](NC(=O)OC)C(C)(C)C(F)(F)F)C(=O)O ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(C)[C@@H](C)C(=O)O)cc(C)cc1OP(=O)(O)O)C(C)(C)C(F)(F)F]

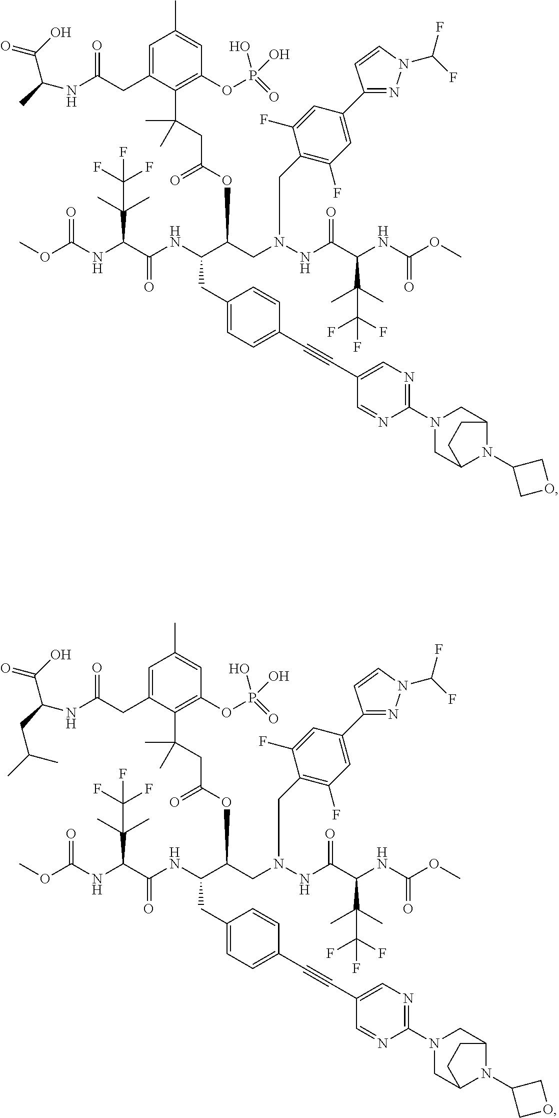 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](C)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CC(C)C)C(=O)O)cc(C)cc1OP(=O)(O)O)C(C)(C)C(F)(F)F]

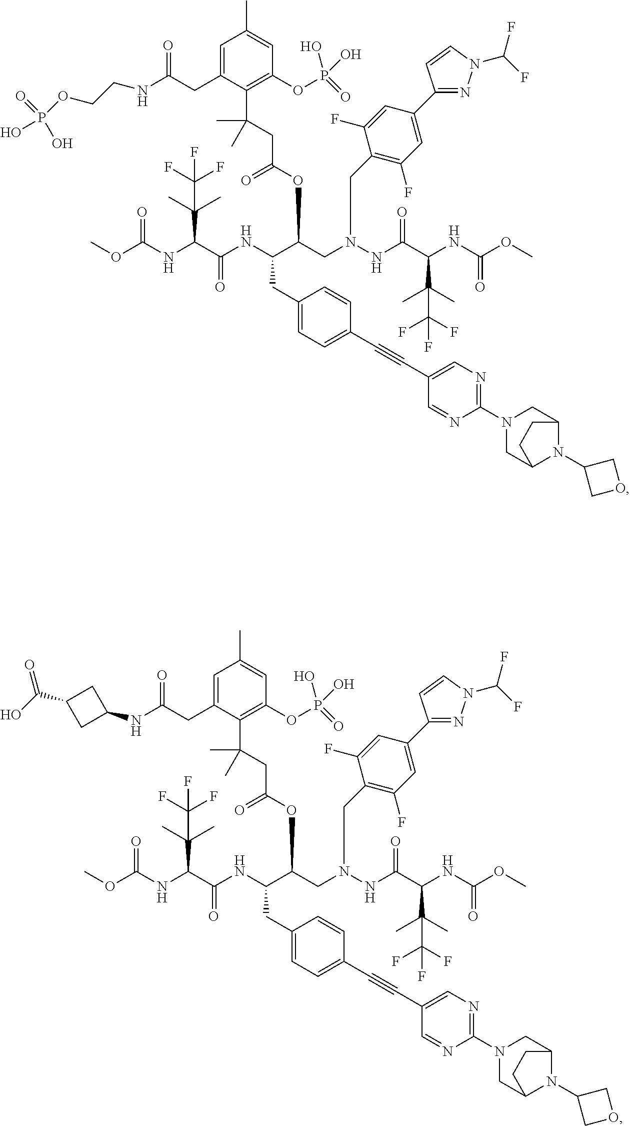 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCOP(=O)(O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H]2C[C@H](C(=O)O)C2)cc(C)cc1OP(=O)(O)O)C(C)(C)C(F)(F)F]

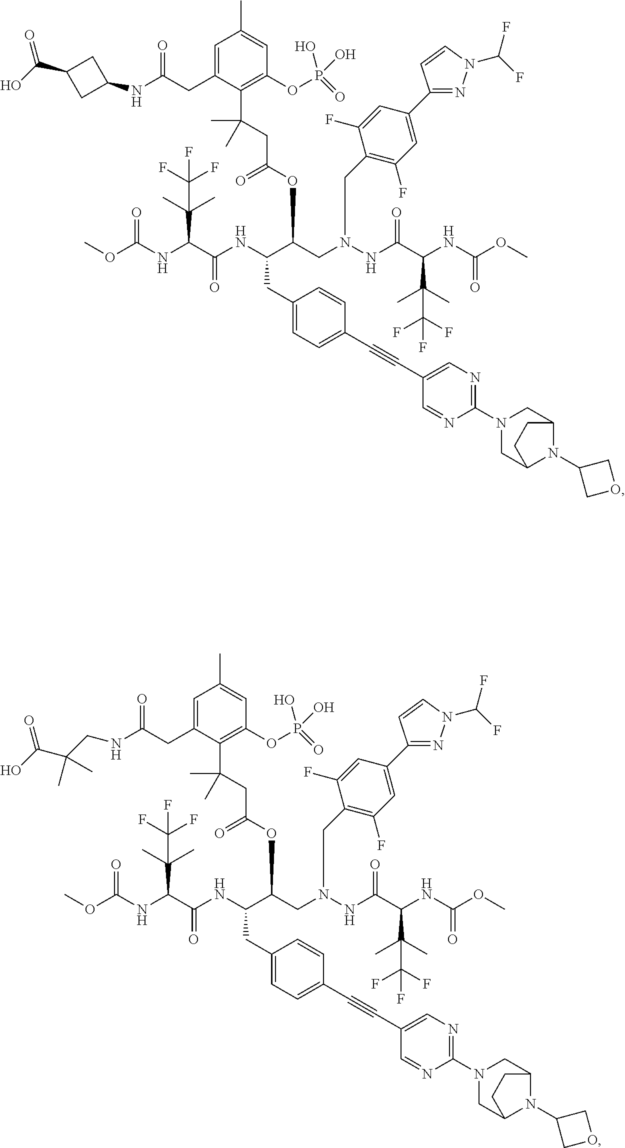 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H]2C[C@@H](C(=O)O)C2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(C)(C)C(=O)O)cc(C)cc1OP(=O)(O)O)C(C)(C)C(F)(F)F]

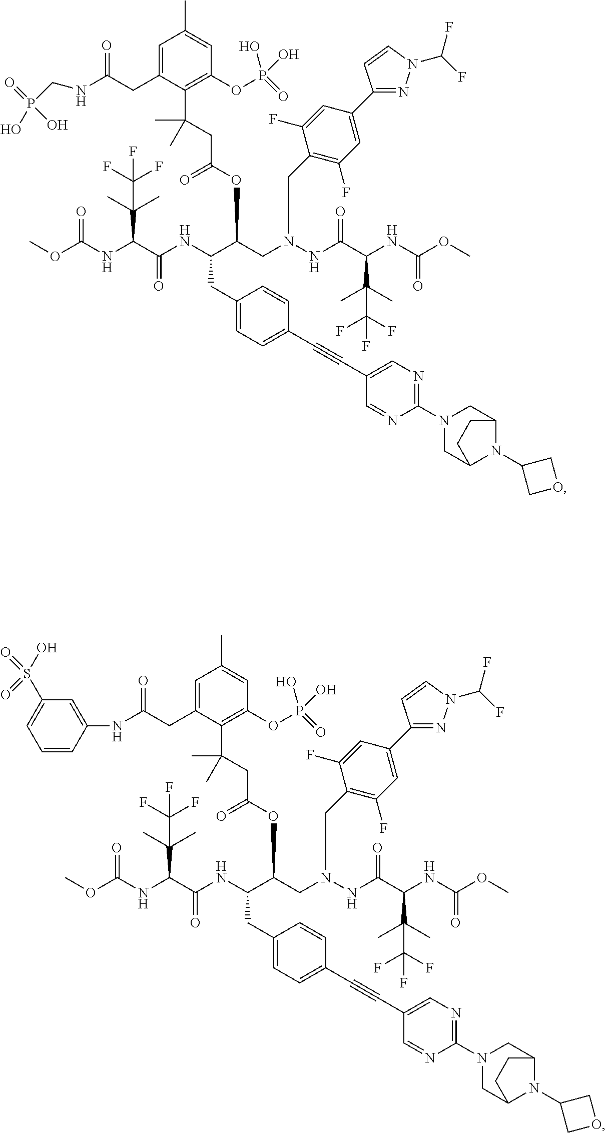 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCP(=O)(O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(S(=O)(=O)O)c2)cc(C)cc1OP(=O)(O)O)C(C)(C)C(F)(F)F]

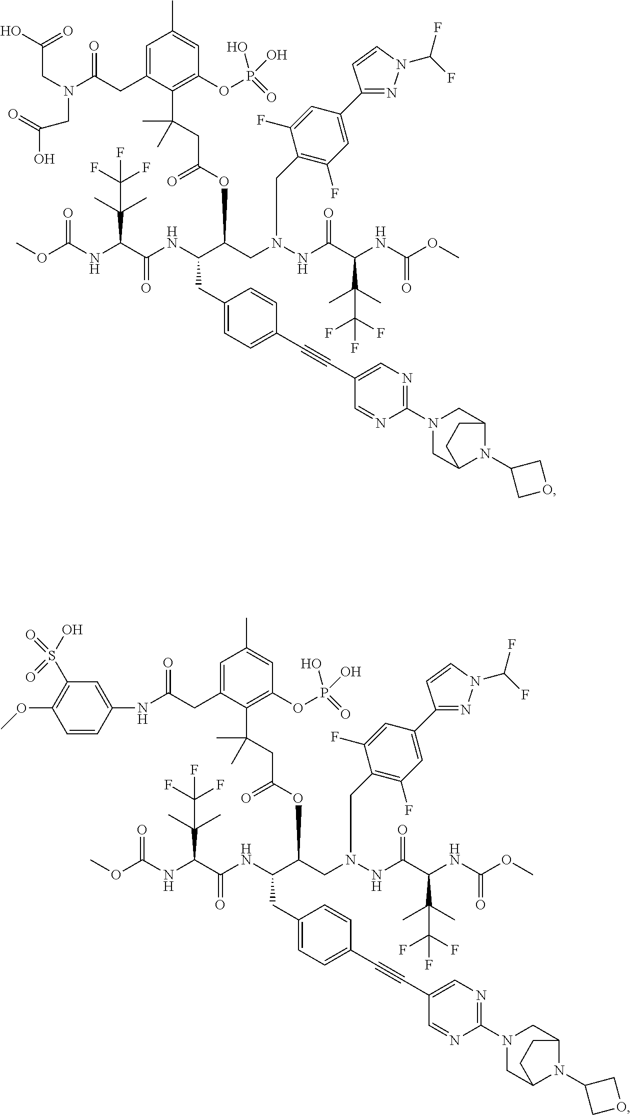 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(CC(=O)O)CC(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccc(OC)c(S(=O)(=O)O)c2)cc(C)cc1OP(=O)(O)O)C(C)(C)C(F)(F)F]

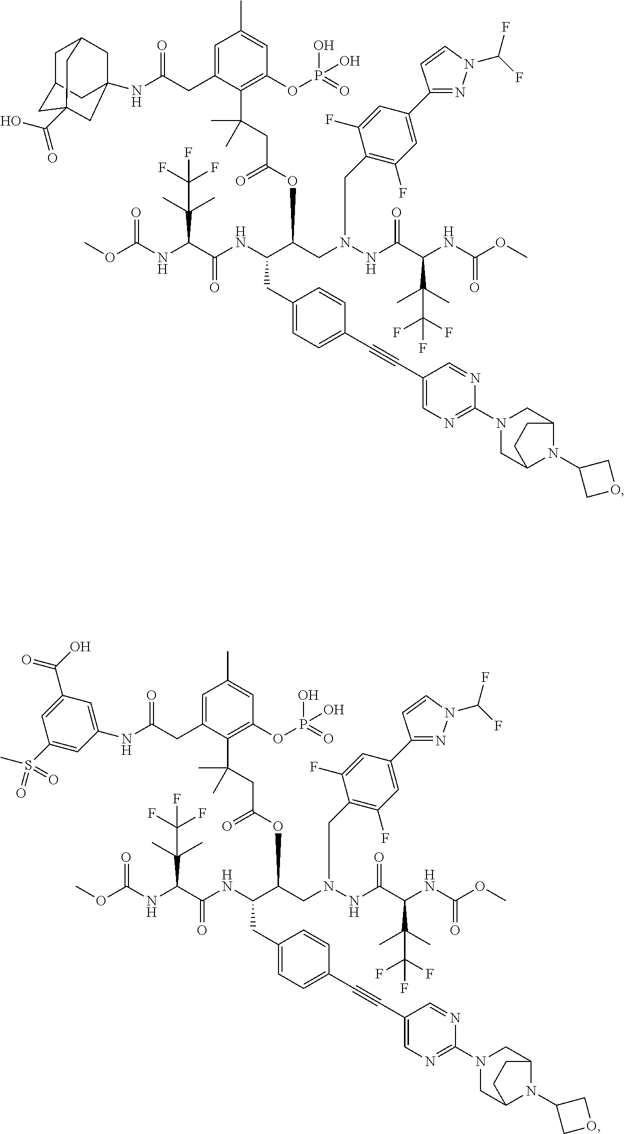 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC23CC4CC(C2)CC(C(=O)O)(C4)C3)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)O)cc(S(C)(=O)=O)c2)cc(C)cc1OP(=O)(O)O)C(C)(C)C(F)(F)F]

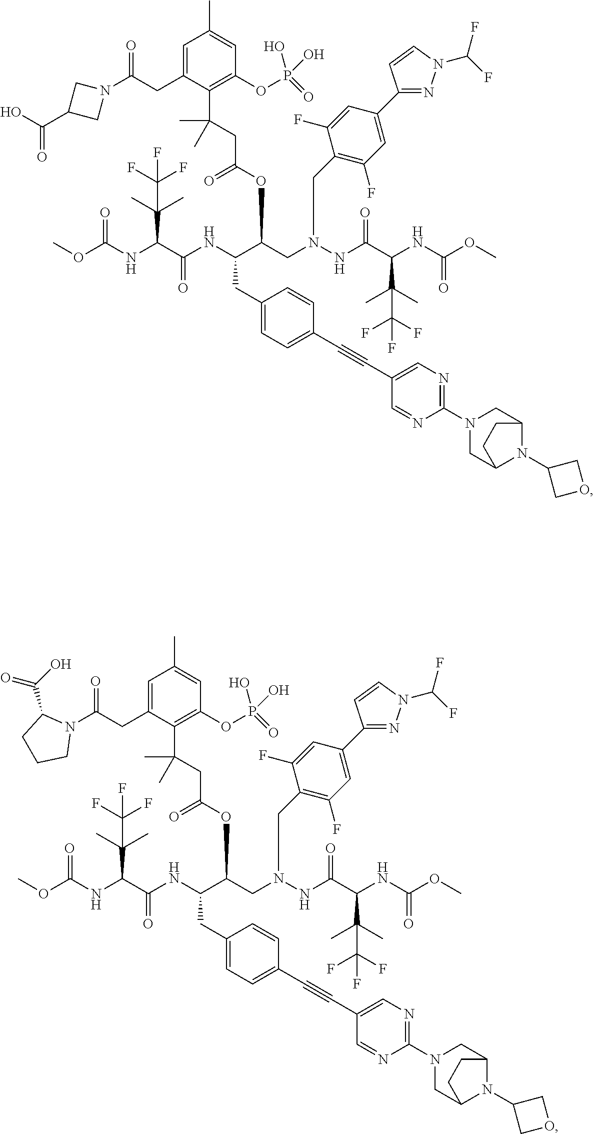 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC(C(=O)O)C2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@@H]2C(=O)O)cc(C)cc1OP(=O)(O)O)C(C)(C)C(F)(F)F]

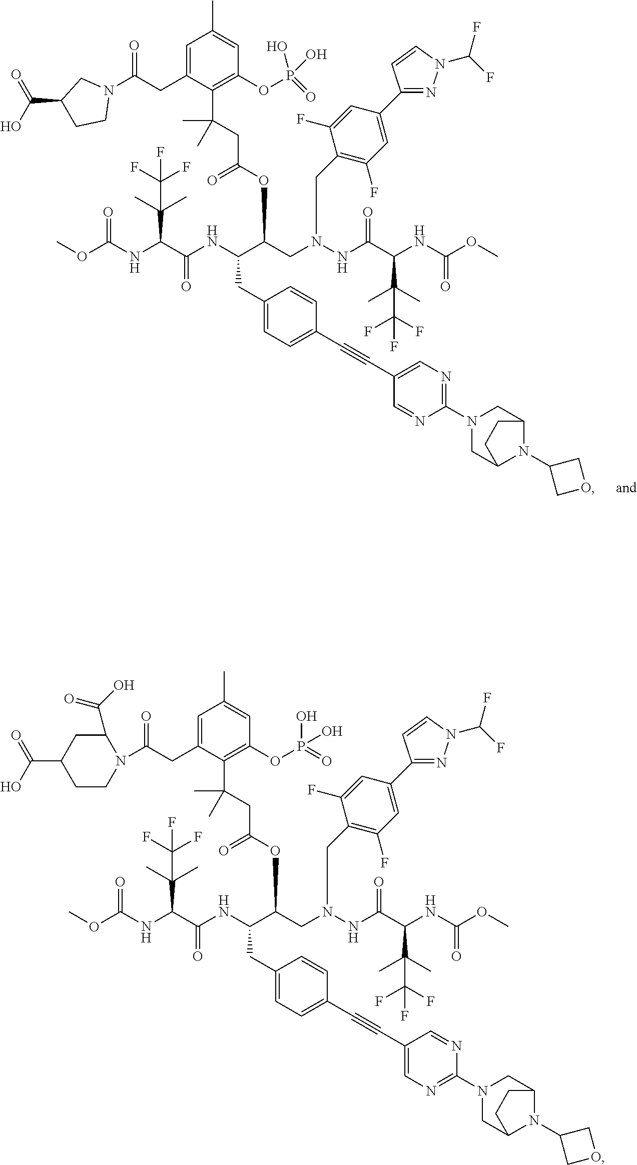 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC[C@@H](C(=O)O)C2)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC(C(=O)O)CC2C(=O)O)cc(C)cc1OP(=O)(O)O)C(C)(C)C(F)(F)F]

or a pharmaceutically acceptable salt thereof.

In certain embodiments, the compound is:

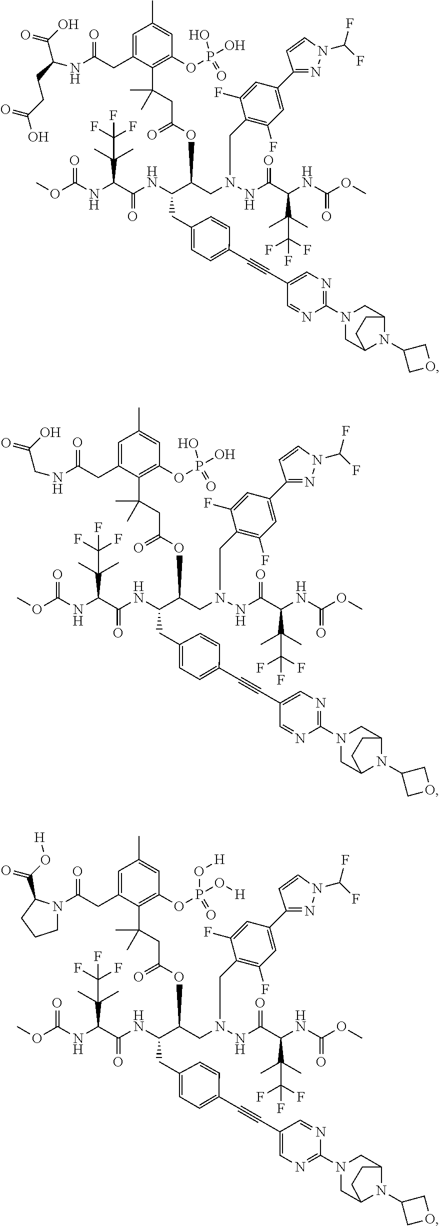

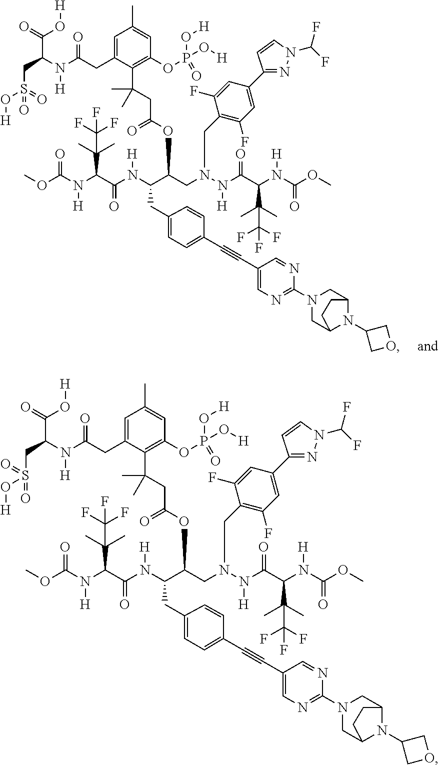 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F ; "and" ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

or a pharmaceutically acceptable salt thereof.

In certain embodiments, the compound is:

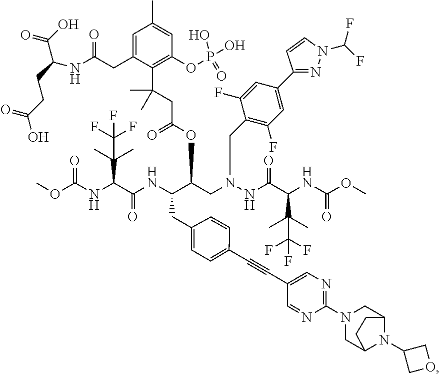 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cc(C)cc1OP(=O)(O)O)C(C)(C)C(F)(F)F]

or a pharmaceutically acceptable salt thereof.

In certain embodiments, the compound is:

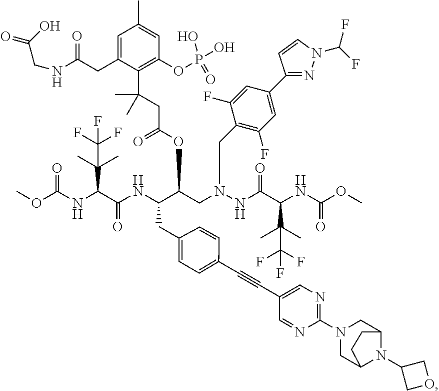 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)O)cc(C)cc1OP(=O)(O)O)C(C)(C)C(F)(F)F]

or a pharmaceutically acceptable salt thereof.

In certain embodiments, the compound is:

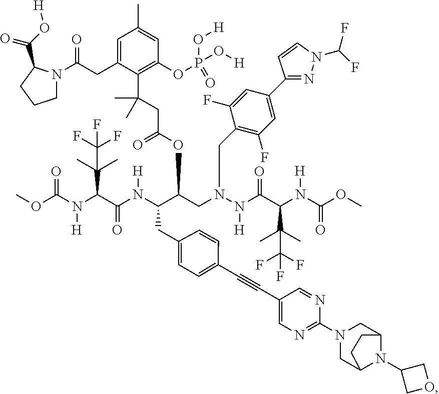 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@H]2C(=O)O)cc(C)cc1OP(=O)(O)O)C(C)(C)C(F)(F)F]

or a pharmaceutically acceptable salt thereof.

In certain embodiments, the compound is:

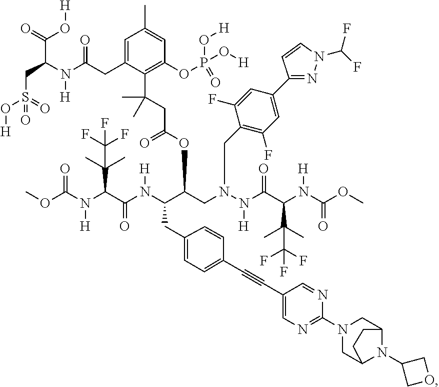 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

or a pharmaceutically acceptable salt thereof.

In certain embodiments, the compound is:

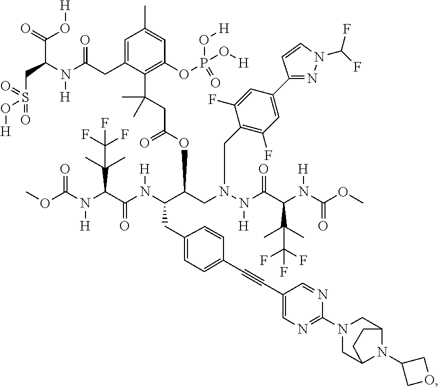 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

or a pharmaceutically acceptable salt thereof.

In some embodiments, the compounds provided herein are prodrugs of compounds having the formula:

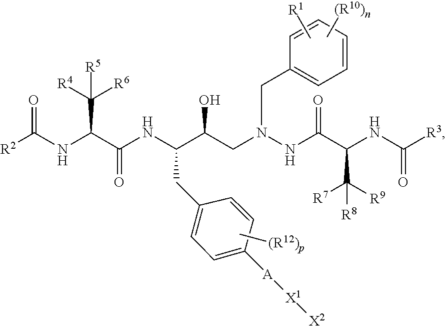

or a pharmaceutically acceptable salt thereof.

### Methods of Treatment

The pharmaceutical compositions of compounds described herein may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In one aspect, the compounds described herein may be administered orally. Oral administration may be via, for example, capsule or enteric coated tablets. In making the pharmaceutical compositions that include at least one compound described herein, is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound, can be formulated so as to provide quick, sustained or delayed release of the active ingredient after administration to the subject by employing procedures known in the art. Controlled-release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions may, in some embodiments, be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In some embodiments, for oral administration, each dosage unit contains from about 10 mg to about 1000 mg of a compound described herein, for example from about 50 mg to about 500 mg, for example about 50 mg, about 75 mg, about 100 mg, about 150 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In other embodiments, for parenteral administration, each dosage unit contains from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, and the severity of the subject's symptoms.

In certain embodiments, dosage levels may be from 0.1 mg to 100 mg per kilogram of body weight per day, for example from about 1 mg to about 50 mg per kilogram, for example from about 5 mg to about 30 mg per kilogram. Such dosage levels may, in certain instances, be useful in the treatment of the above-indicated conditions. In other embodiments, dosage levels may be from about 10 mg to about 2000 mg per subject per day. The amount of active ingredient that may be combined with the vehicle to produce a single dosage form will vary depending upon the host treated and the particular mode of administration. Dosage unit forms may contain from 1 mg to 1000 mg of an active ingredient.

The compounds disclosed herein, or a pharmaceutically acceptable salt thereof, may be administered to a subject in accordance with an effective dosing regimen for a desired period of time or duration, such as at least about one day, at least about one week, at least about one month, at least about 2 months, at least about 3 months, at least about 4 months, at least about 6 months, or at least about 12 months or longer. In one variation, the compound is administered on a daily or intermittent schedule. In one variation, the compound is administered on a monthly schedule. In one variation, the compound is administered every two months. In one variation, the compound is administered every three months. In one variation, the compound is administered every four months. In one variation, the compound is administered every five months. In one variation, the compound is administered every 6 months.

The dosage or dosing frequency of a compound disclosed herein, or a pharmaceutically acceptable salt thereof, may be adjusted over the course of the treatment, based on the judgment of the administering physician. The compound may be administered to a subject (e.g., a human) in an effective amount. In certain embodiments, the compound is administered once daily.

For preparing solid compositions such as tablets, the principal active ingredient may be mixed with a pharmaceutical excipient to form a solid preformulation composition containing a homogeneous mixture of a compound. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6th edition 2009.

In some embodiments, the compounds described herein, or a pharmaceutically acceptable salt thereof, may be administered with a syringe. In some embodiments, the syringe is disposable. In some embodiments, the syringe is reusable. In some embodiments, the syringe is pre-filled with a compound described herein, or a pharmaceutically acceptable salt thereof.

In some embodiments, the compounds described herein, or a pharmaceutically acceptable salt thereof, may be administered with an auto-injector comprising a syringe. In some embodiments, the syringe is disposable. In some embodiments, the syringe is reusable. In some embodiments, the syringe is pre-filled with a compound described herein, or a pharmaceutically acceptable salt thereof.

In certain embodiments, a method of treating or preventing a Retroviridae viral infection (e.g., a human immunodeficiency virus (HIV) infection) comprising administering a therapeutically effective amount of a compound disclosed herein, or a pharmaceutically acceptable salt thereof, to a subject in need thereof, is provided. In certain embodiments, a method of treating a human immunodeficiency virus (HIV) infection comprising administering a therapeutically effective amount of a compound disclosed herein, or a pharmaceutically acceptable salt thereof, to a subject in need thereof, is provided. In certain embodiments, a method of treating a human immunodeficiency virus (HIV) infection comprising administering a therapeutically effective amount of a compound disclosed herein, or a pharmaceutically acceptable salt thereof, to a patient in need thereof, is provided. In certain embodiments, a method of treating a human immunodeficiency virus (HIV) infection comprising administering a therapeutically effective amount of a compound disclosed herein, or a pharmaceutically acceptable salt thereof, to a heavily treatment-experienced patient in need thereof, is provided.

In certain embodiments, the method comprises administering a compound disclosed herein, or a pharmaceutically acceptable salt thereof, in combination with one, two, three, or four additional therapeutic agents. In certain embodiments, the subject is at risk of contracting the HIV virus, such as a subject who has one or more risk factors known to be associated with contracting the HIV virus. In certain embodiments, the subject may have not previously received antiviral treatment (treatment naïve). In certain embodiments, the subject may have previously received antiviral treatment (treatment experienced). In certain embodiments, the subject may have previously received antiviral treatment and developed resistance to the previously received antiviral treatment.

In certain embodiments, a method of treating or preventing a Retroviridae viral infection (e.g., a human immunodeficiency virus (HIV) infection) comprising administering a therapeutically effective amount of a compound disclosed herein, or a pharmaceutically acceptable salt thereof, to a subject in need thereof, in combination with a therapeutically effective amount of one or more (e.g., one, two, three, or four; or one or two; or one to three; or one to four) additional therapeutic agents selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof, is provided. In certain embodiments, the one or more (e.g., one, two, three, or four; or one or two; or one to three; or one to four) additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, or any combinations thereof. In certain embodiments, the one or more additional therapeutic agent does not include a pharmacokinetic enhancer.

In certain embodiments, the one, two, three, or four additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, and combinations thereof.

In certain embodiments, the one, two, three, or four additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, and “antibody-like” therapeutic proteins, or any combinations thereof.

In certain embodiments, a method for inhibiting the replication of the HIV virus, treating AIDS or delaying the onset of AIDS in a subject (e.g., a human), comprising administering a compound disclosed herein, or a pharmaceutically acceptable salt thereof, to the subject is disclosed.

In certain embodiments, a compound of disclosed herein, or a pharmaceutically acceptable salt thereof for use in medical therapy of an HIV infection (e.g., HIV-1 or the replication of the HIV virus (e.g., HIV-1) or AIDS or delaying the onset of AIDS in a subject (e.g., a human)) is disclosed.

In certain embodiments, a compound disclosed herein, or a pharmaceutically acceptable salt thereof for use in the manufacture of a medicament for treating an HIV infection or the replication of the HIV virus or AIDS or delaying the onset of AIDS in a subject (e.g., a human) is disclosed. One embodiment relates to a compound disclosed herein, or a pharmaceutically acceptable salt thereof, for use in the prophylactic or therapeutic treatment of an HIV infection or AIDS or for use in the therapeutic treatment or delaying the onset of AIDS.

In certain embodiments, the use of a compound disclosed herein, or a pharmaceutically acceptable salt thereof, for the manufacture of a medicament for an Retroviridae viral infection (e.g., an HIV infection) in a subject (e.g., a human) is disclosed. In certain embodiments, a compound disclosed herein, or a pharmaceutically acceptable salt thereof, for use in the prophylactic or therapeutic treatment of an HIV infection is disclosed.

In certain embodiments, in the methods of use, the administration is to a subject (e.g., a human) in need of the treatment. In certain embodiments, in the methods of use, the administration is to a subject (e.g., a human) who is at risk of developing AIDS.

The compounds disclosed herein, or a pharmaceutically acceptable salt thereof, for use in therapy is provided. In one embodiment, a compound disclosed herein, or a pharmaceutically acceptable salt thereof, is for use in a method of treating or preventing an HIV infection or the replication of the HIV virus or AIDS or delaying the onset of AIDS in a subject (e.g., a human).

The compounds disclosed herein, or a pharmaceutically acceptable salt thereof, for use in a method of treating or preventing a Retroviridae viral infection (e.g., an HIV infection) in a subject in need thereof is provided. In certain embodiments, a compound disclosed herein, or a pharmaceutically acceptable salt thereof, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In one embodiment, a compound disclosed herein, or a pharmaceutically acceptable salt thereof, in combination with one or more (e.g., one, two, three, or four; or one or two; or one to three; or one to four) additional therapeutic agents as described herein for use in a method of treating or preventing HIV infection in a subject in need thereof is provided. In one embodiment, the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof. In certain embodiments, the additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, or any combinations thereof.

In one embodiment, a compound disclosed herein, or a pharmaceutically acceptable salt thereof, in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine, is provided for use in a method of treating or preventing HIV infection in a subject in need thereof. In a particular embodiment, a compound disclosed herein, or a pharmaceutically acceptable salt thereof, in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine, is provided for use in a method of treating or preventing HIV infection in a subject in need thereof.

In certain embodiments, the one, two, three, or four additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, and tenofovir alafenamide hemifumarate, or a pharmaceutically acceptable salt thereof.

In certain embodiments, the one, two, three, or four additional therapeutic agents are selected from abacavir sulfate, bictegravir, tenofovir, tenofovir disoproxil, tenofovir disoproxil fumarate, tenofovir disoproxil hemifumarate, tenofovir alafenamide, tenofovir alafenamide fumarate, and tenofovir alafenamide hemifumarate.

In certain embodiments, the one, two, three, or four additional therapeutic agents are selected from tenofovir alafenamide, tenofovir alafenamide fumarate, and tenofovir alafenamide hemifumarate.

In certain embodiments, the one, two, three, or four additional therapeutic agents are selected from bictegravir, emtricitabine, and GS-9131.

In a particular embodiment, a compound disclosed herein or a pharmaceutically acceptable salt thereof, are provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a compound disclosed herein, or a pharmaceutically acceptable salt thereof. In certain embodiments, methods for reducing the risk of acquiring HIV (e.g., HIV-1 and/or HIV-2) comprise administration of a compound disclosed herein, or a pharmaceutically acceptable salt thereof, in combination with one or more additional therapeutic agents. In certain embodiments, methods for reducing the risk of acquiring HIV (e.g., HIV-1 and/or HIV-2) comprise administration of a pharmaceutical composition comprising a therapeutically effective amount of the compound disclosed herein, or pharmaceutically acceptable salt thereof, and a pharmaceutically acceptable excipient.

In certain embodiments, methods for reducing the risk of acquiring HIV (e.g., HIV-1 and/or HIV-2) comprise administration of a compound of disclosed herein, or a pharmaceutically acceptable salt thereof, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In certain embodiments, the reduction in risk of acquiring HIV is at least about 40%, 50%, 60%, 70%, 80%, 90%, or 95%. In certain embodiments, the reduction in risk of acquiring HIV is at least about 75%. In certain embodiments, the reduction in risk of acquiring HIV is about 80%, 85%, or 90%.

In another embodiment, the use of a compound disclosed herein, or a pharmaceutically acceptable salt thereof, for the manufacture of a medicament for the treatment of an HIV infection in a human being having or at risk of having the infection is disclosed.

Also disclosed herein is a compound disclosed herein, or a pharmaceutically acceptable salt thereof, for use in the therapeutic treatment or delaying the onset of AIDS.

Also disclosed herein is a compound disclosed herein, or a pharmaceutically acceptable salt thereof, for use in the prophylactic or therapeutic treatment of an HIV infection.

In certain embodiments, a compound disclosed herein, or a pharmaceutically acceptable salt thereof can be used as a research tool (e.g., to study the inhibition of HIV reverse transcriptase in a subject or in vitro).

Kits that include a compound of Formula (I), Formula (II), Formula (III), Formula (IV), Formula (V), Formula (VI), Formula (VII), Formula (VIII), Formula (IX), Formula (X), and/or Formula (XI), or a pharmaceutically acceptable salt, thereof, and suitable packaging are provided. In one embodiment, a kit further includes instructions for use. In one aspect, a kit includes a compound of Formula (I), or a pharmaceutically acceptable salt thereof, and instructions for use of the compounds in the treatment of the diseases or conditions described herein.

Articles of manufacture that include a compound of Formula (I), Formula (II), Formula (III), Formula (IV), Formula (V), Formula (VI), Formula (VII), Formula (VIII), Formula (IX), Formula (X), and/or Formula (XI), or a pharmaceutically acceptable salt thereof, in a suitable container are provided. The container may be a vial, jar, ampoule, preloaded syringe, and intravenous bag.

### Administration of HIV Combination Therapy

In certain embodiments, a compound disclosed herein is administered with one or more additional therapeutic agents. Co-administration of a compound disclosed herein with one or more additional therapeutic agents generally refers to simultaneous or sequential administration of a compound disclosed herein and one or more additional therapeutic agents, such that therapeutically effective amounts of the compound disclosed herein and the one or more additional therapeutic agents are both present in the body of the patient. When administered sequentially, the combination may be administered in two or more administrations.

Co-administration includes administration of unit dosages of the compounds disclosed herein before or after administration of unit dosages of one or more additional therapeutic agents. For example, the compound disclosed herein may be administered within seconds, minutes, or hours of the administration of the one or more additional therapeutic agents. In some embodiments, a unit dose of a compound disclosed herein is administered first, followed within seconds or minutes by administration of a unit dose of one or more additional therapeutic agents. Alternatively, a unit dose of one or more additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of a compound disclosed herein.

In certain embodiments, a compound disclosed herein is combined with one or more additional therapeutic agents in a unitary dosage form for simultaneous administration to a patient, for example as a solid dosage form for oral administration.

In certain embodiments, a compound is formulated as a tablet, which may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In some embodiments, a compound is formulated as a tablet, which may optionally contain one or more other compounds useful for treating HIV. In certain embodiments, the tablet can contain another active ingredient for treating HIV, such as compounds that target the HIV capsid, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In some embodiments, the compounds that target the HIV capsid are selected from the group consisting of:

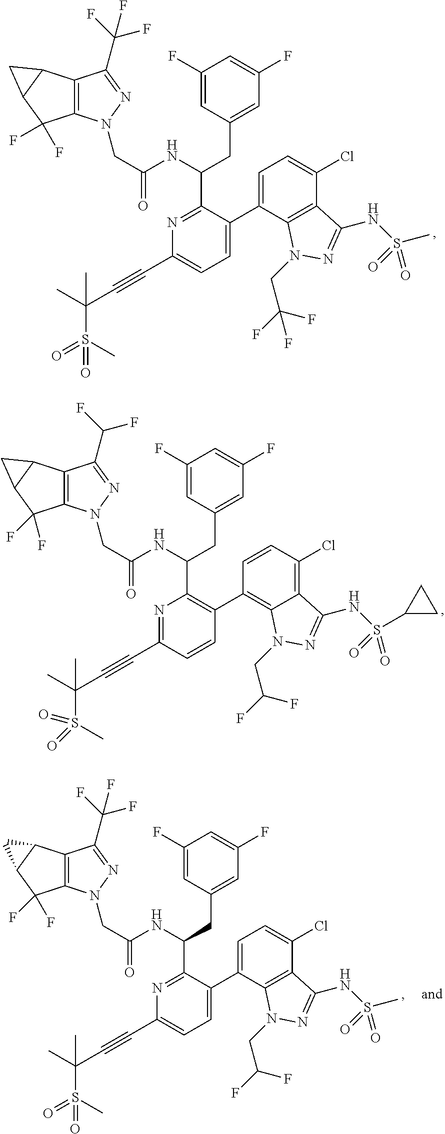

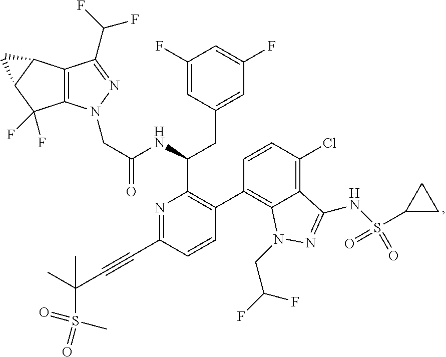 [SMILES: CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

or a pharmaceutically acceptable salt thereof.

In certain embodiments, the one, two, three, or four additional therapeutic agents are selected from:

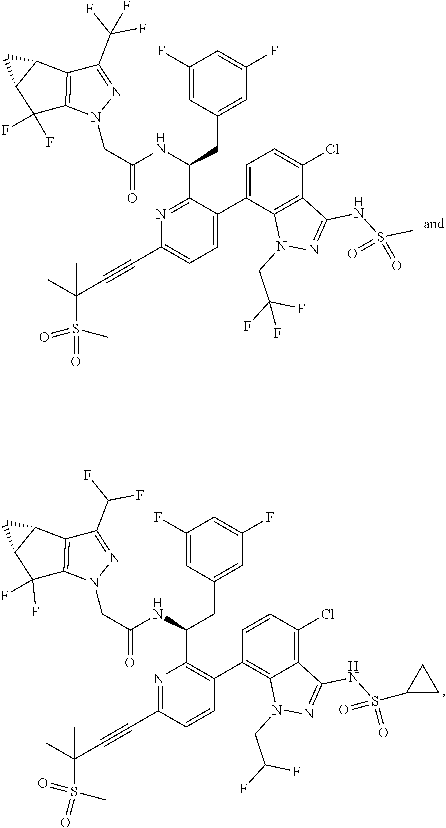 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O ; CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

or a pharmaceutically acceptable salt thereof.

In certain embodiments, the one, two, three, or four additional therapeutic agent is:

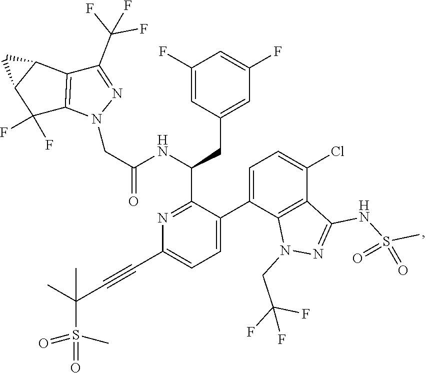 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof.

In certain embodiments, the one, two, three, or four additional therapeutic agent is:

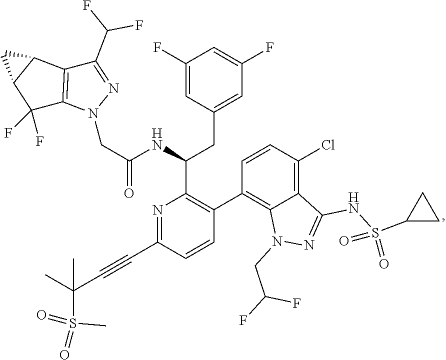 [SMILES: CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

or a pharmaceutically acceptable salt thereof.

In certain embodiments, the one, two, three, or four additional therapeutic agent is:

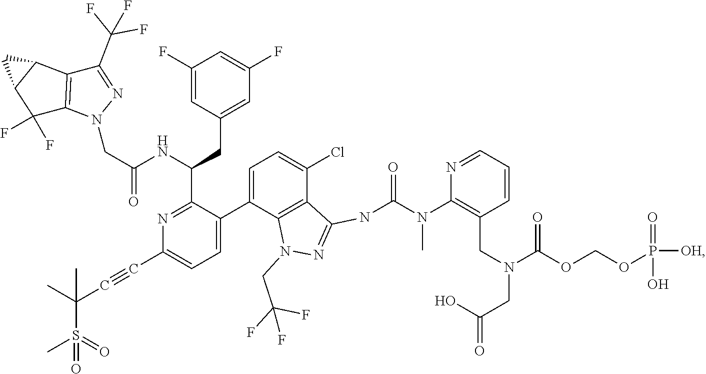 [SMILES: CN(C(=O)Nc1nn(CC(F)(F)F)c2c(-c3ccc(C#CC(C)(C)S(C)(=O)=O)nc3[C@H](Cc3cc(F)cc(F)c3)NC(=O)Cn3nc(C(F)(F)F)c4c3C(F)(F)[C@@H]3C[C@H]43)ccc(Cl)c12)c1ncccc1CN(CC(=O)O)C(=O)OCOP(=O)(O)O]

or a pharmaceutically acceptable salt thereof.

In certain embodiments, the one, two, three, or four additional therapeutic agent is:

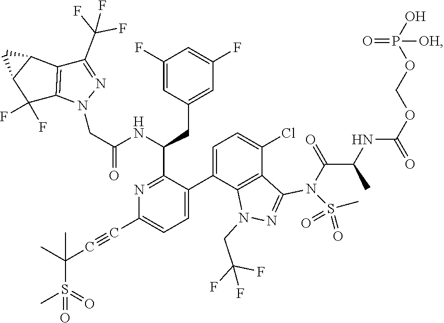 [SMILES: C[C@H](NC(=O)OCOP(=O)(O)O)C(=O)N(c1nn(CC(F)(F)F)c2c(-c3ccc(C#CC(C)(C)S(C)(=O)=O)nc3[C@H](Cc3cc(F)cc(F)c3)NC(=O)Cn3nc(C(F)(F)F)c4c3C(F)(F)[C@@H]3C[C@H]43)ccc(Cl)c12)S(C)(=O)=O]

or a pharmaceutically acceptable salt thereof.

In certain embodiments, the one, two, three, or four additional therapeutic agent is:

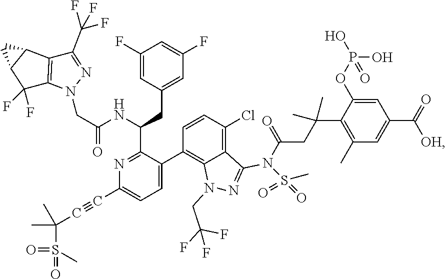 [SMILES: Cc1cc(C(=O)O)cc(OP(=O)(O)O)c1C(C)(C)CC(=O)N(c1nn(CC(F)(F)F)c2c(-c3ccc(C#CC(C)(C)S(C)(=O)=O)nc3[C@H](Cc3cc(F)cc(F)c3)NC(=O)Cn3nc(C(F)(F)F)c4c3C(F)(F)[C@@H]3C[C@H]43)ccc(Cl)c12)S(C)(=O)=O]

or a pharmaceutically acceptable salt thereof.

In certain embodiments, the one, two, three, or four additional therapeutic agent is:

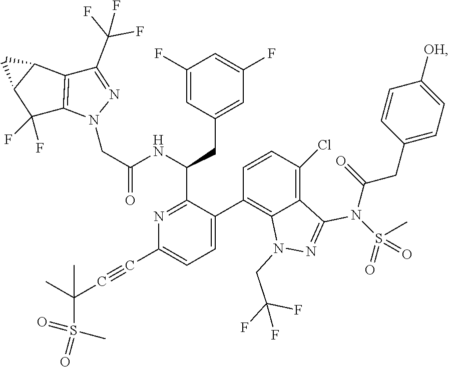 [SMILES: CC(C)(C#Cc1ccc(-c2ccc(Cl)c3c(N(C(=O)Cc4ccc(O)cc4)S(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof.

In certain embodiments, the one, two, three, or four additional therapeutic agent is:

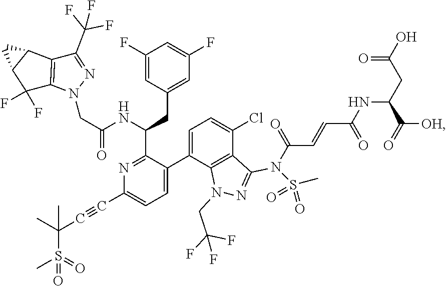 [SMILES: CC(C)(C#Cc1ccc(-c2ccc(Cl)c3c(N(C(=O)/C=C/C(=O)N[C@@H](CC(=O)O)C(=O)O)S(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof.

In certain embodiments, the one, two, three, or four additional therapeutic agent is:

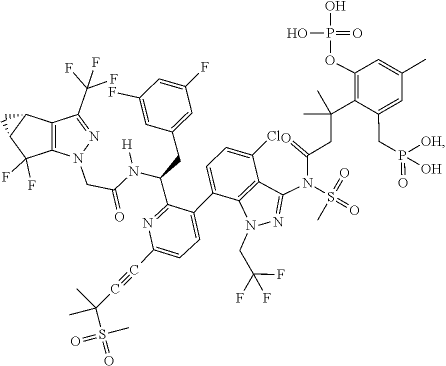 [SMILES: Cc1cc(CP(=O)(O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof.

In certain embodiments, the one, two, three, or four additional therapeutic agent is:

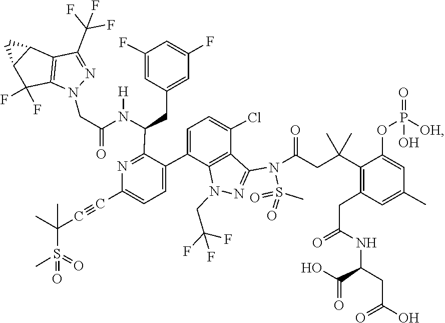 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof.

In certain embodiments, the one, two, three, or four additional therapeutic agent is:

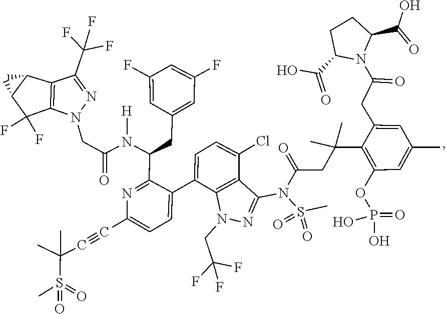 [SMILES: Cc1cc(CC(=O)N2[C@H](C(=O)O)CC[C@H]2C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof.

In certain embodiments, such tablets are suitable for once daily dosing.

### HIV Combination Therapy

In some embodiments, provided herein is a method for preventing or treating an HIV infection, comprising administering to a patient in need thereof a therapeutically effective amount of a compound disclosed herein, or a pharmaceutically acceptable salt thereof, in combination with a therapeutically effective amount of one or more additional therapeutic agents which are suitable for treating an HIV infection.

In the above embodiments, the additional therapeutic agent may be an anti-HIV agent. For example, in some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent is selected from immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies such as chimeric antigen receptor T-cell, CAR-T (e.g., YESCARTA® (axicabtagene ciloleucel)), and engineered T cell receptors, TCR-T.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof. HIV Combination Drugs

Examples of combination drugs include ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; Vacc-4x and romidepsin; and APH-0812.

### Other HIV Drugs

Examples of other drugs for treating HIV include acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, ABX-464, AG-1105, APH-0812, BIT-225, CYT-107, HGTV-43, HPH-116, HS-10234, IMO-3100, IND-02, MK-1376, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

### HIV Protease Inhibitors

Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, and TMC-310911.

### HIV Reverse Transcriptase Inhibitors

Examples of HIV non-nucleoside or non-nucleotide inhibitors of reverse transcriptase include dapivirine, delavirdine, delavirdine mesylate, doravirine, efavirenz, etravirine, lentinan, nevirapine, rilpivirine, AIC-292, KM-023, and VM-1500.

In some embodiments, examples of HIV non-nucleoside or non-nucleotide inhibitors of reverse transcriptase include dapivirine, delavirdine, delavirdine mesylate, doravirine, efavirenz, etravirine, lentinan, nevirapine, rilpivirine, AIC-292, KM-023, PC-1005, and VM-1500.

Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, GS-9131, GS-9148, and KP-1461.

### HIV Integrase Inhibitors

Examples of HIV integrase inhibitors include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169 and cabotegravir.

Examples of HIV non-catalytic site, or allosteric, integrase inhibitors (NCINI) include CX-05045, CX-05168, and CX-14442.

### HIV Entry Inhibitors

Examples of HIV entry (fusion) inhibitors include cenicriviroc, CCR5 inhibitors, gp41 inhibitors, CD4 attachment inhibitors, gp120 inhibitors, and CXCR4 inhibitors.

Examples of CCR5 inhibitors include aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

Examples of gp41 inhibitors include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

Examples of CD4 attachment inhibitors include ibalizumab and CADA analogs.

Examples of gp120 inhibitors include Radha-108 (receptol) 3B3-PE38, BanLec, bentonite-based nanomedicine, fostemsavir tromethamine, IQP-0831, and BMS-663068.

Examples of CXCR4 inhibitors include plerixafor, ALT-1188, N15 peptide, and vMIP (Haimipu).

### HIV Maturation Inhibitors

Examples of HIV maturation inhibitors include BMS-955176 and GSK-2838232.

### Latency Reversing Agents

Examples of latency reversing agents include histone deacetylase (HDAC) inhibitors, proteasome inhibitors such as velcade, protein kinase C (PKC) activators, BET-bromodomain 4 (BRD4) inhibitors, ionomycin, PMA, SAHA (suberanilohydroxamic acid, or suberoyl, anilide, and hydroxamic acid), IL-15, JQ1, disulfram, amphotericin B, and ubiquitin inhibitors such as largazole analogs, and GSK-343.

Examples of HDAC inhibitors include romidepsin, vorinostat, and panobinostat.

Examples of PKC activators include indolactam, prostratin, ingenol B, and DAG-lactones.

### HIV Capsid Inhibitors

Examples of capsid inhibitors include capsid polymerization inhibitors or capsid disrupting compounds, HIV nucleocapsid p7 (NCp7) inhibitors such as azodicarbonamide, HIV p24 capsid protein inhibitors, AVI-621, AVI-101, AVI-201, AVI-301, and AVI-CAN1-15 series;

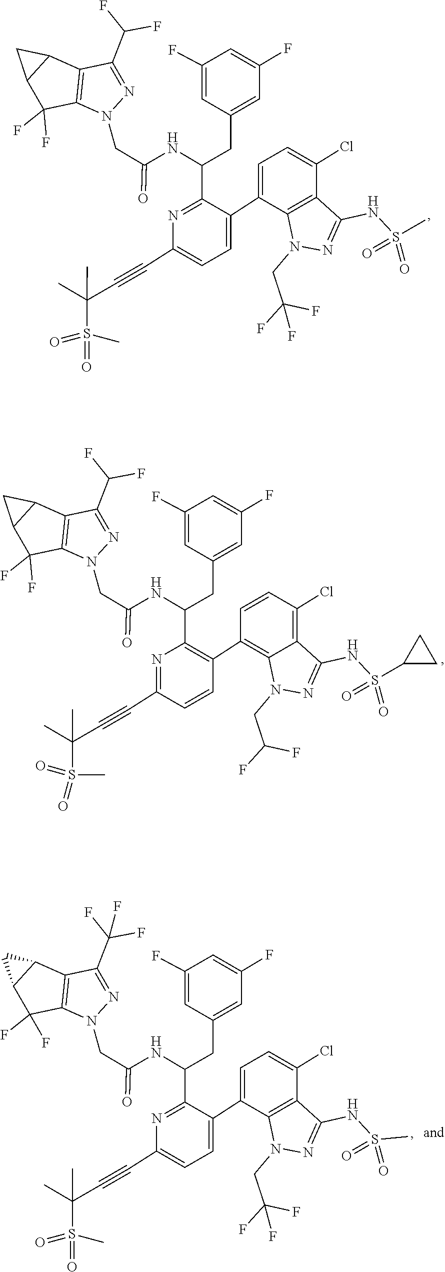 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)F)c3c2C(F)(F)C2CC32)n1)S(C)(=O)=O ; CC(C)(C#Cc1ccc(-c2ccc(Cl)c3c(NS(=O)(=O)C4CC4)nn(CC(F)F)c23)c(C(Cc2cc(F)cc(F)c2)NC(=O)Cn2nc(C(F)F)c3c2C(F)(F)C2CC32)n1)S(C)(=O)=O ; CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)[C@@H]2C[C@H]32)n1)S(C)(=O)=O]

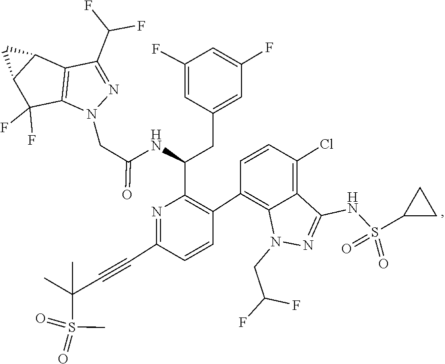 [SMILES: CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

In some embodiments, examples of capsid inhibitors include:

or a pharmaceutically acceptable salt thereof.

In some embodiments, the capsid inhibitor is selected from:

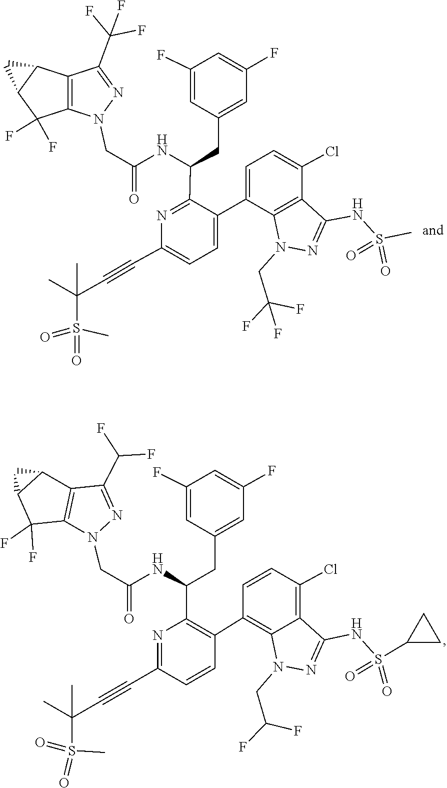

or a pharmaceutically acceptable salt thereof.

In some embodiments, the capsid inhibitor is:

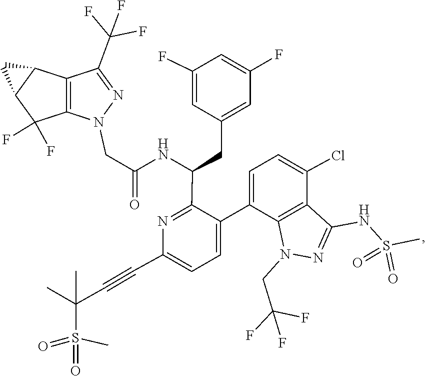 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

In some embodiments, the capsid inhibitor is:

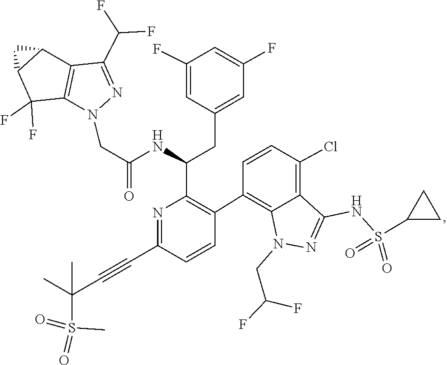

or a pharmaceutically acceptable salt thereof.

### Immune-Based Therapies

Examples of immune-based therapies include toll-like receptors modulators such as tlr1, tlr2, tlr3, tlr4, tlr5, tlr6, tlr7, tlr8, tlr9, tlr1, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Ec fusion protein, normferon, peginterferon alfa-2a, peginterferon alfa-2b, recombinant interleukin-15, RPI-MN, GS-9620, and IR-103.

In some embodiments, examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fe fusion protein, normferon, peginterferon alfa-2a, peginterferon alfa-2b, recombinant interleukin-15, RPI-MN, GS-9620, STING modulators, RIG-I modulators, NOD2 modulators, and IR-103.

### Phosphatidylinositol 3-Kinase (PI3K) Inhibitors

Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include PTG-100, TRK-170, abrilumab, etrolizumab, carotegrast methyl, and vedolizumab.

### HIV Antibodies, Bispecific Antibodies, and “Antibody-Like” Therapeutic Proteins

Examples of HIV antibodies, bispecific antibodies, and “antibody-like” therapeutic proteins include DARTs®, DUOBODIES®, BITES®, XmAbs®, TandAbs®, Fab derivatives, bnABs (broadly neutralizing HIV-1 antibodies), BMS-936559, TMB-360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, MB-66.

In some embodiments, examples of those targeting HIV in such a manner include bavituximab, UB-421, C2F5, C2G12, C4E10, C2F5+C2G12+C4E10, 3-BNC-117, PGT145, PGT121, MDX010 (ipilimumab), VRC01, A32, 7B2, 10E8, VRC-07-523, VRC-HIVMAB080-00-AB, MGD-014 and VRC07.

In some embodiments, examples of those targeting HIV in such a manner include bavituximab, UB-421, C2F5, 2G12, C4E10, C2F5+C2G12+C4E10, 8ANC195, 3BNC117, 3BNC60, 10-1074, PGT145, PGT121, PGT-151, PGT-133, MDXO10 (ipilimumab), DH511, N6, VRC01 PGDM1400, A32, 7B2, 10E8, 10E8v4, CAP256-VRC26.25, DRVIA7, VRC-07-523, VRC-HIVMAB080-00-AB, VRC-HIVMAB060-00-AB, MGD-014 and VRC07. Example of HIV bispecific antibodies includes MGD014.

### Pharmacokinetic Enhancers

Examples of pharmacokinetic enhancers include cobicistat and ritonavir.

### Additional Therapeutic Agents

Examples of additional therapeutic agents include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

In some embodiments, examples of HIV vaccines include peptide vaccines, recombinant subunit protein vaccines, live vector vaccines, DNA vaccines, CD4-derived peptide vaccines, vaccine combinations, rgp120 (AIDSVAX), ALVAC HIV (vCP1521)/AIDSVAX B/E (gp120) (RV144), monomeric gp120 HIV-1 subtype C vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), Pennvax-G, Pennvax-GP, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), EN41-UGR7C, EN41-FPA2, PreVaxTat, AE-H, MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120, Vacc-4x+romidepsin, variant gp120 polypeptide vaccine, rAd5 gag-pol env A/B/C vaccine, DNA.HTI and MVA.HTI.

### HIV Combination Therapy

In a particular embodiment, a compound disclosed herein, or a pharmaceutically acceptable salt thereof, is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

It will be appreciated by one of skill in the art that the additional therapeutic agents listed above may be included in more than one of the classes listed above. The particular classes are not intended to limit the functionality of those compounds listed in those classes.

In a specific embodiment, a compound disclosed herein, or a pharmaceutically acceptable salt thereof, is combined with an HIV nucleoside or nucleotide inhibitor of reverse transcriptase and an HIV non-nucleoside inhibitor of reverse transcriptase. In another specific embodiment, a compound disclosed herein, or a pharmaceutically acceptable salt thereof, is combined with an HIV nucleoside or nucleotide inhibitor of reverse transcriptase, and an HIV protease inhibiting compound. In an additional embodiment, a compound disclosed herein, or a pharmaceutically acceptable salt thereof, is combined with an HIV nucleoside or nucleotide inhibitor of reverse transcriptase, an HIV non-nucleoside inhibitor of reverse transcriptase, and a pharmacokinetic enhancer. In certain embodiments, a compound disclosed herein, or a pharmaceutically acceptable salt thereof, is combined with at least one HIV nucleoside inhibitor of reverse transcriptase, an integrase inhibitor, and a pharmacokinetic enhancer. In another embodiment, a compound disclosed herein, or a pharmaceutically acceptable salt thereof, is combined with two HIV nucleoside or nucleotide inhibitors of reverse transcriptase.

In a particular embodiment, a compound disclosed herein, or a pharmaceutically acceptable salt thereof, is combined with GS-9131, abacavir sulfate, bictegravir, tenofovir, tenofovir disoproxil, tenofovir disoproxil fumarate, tenofovir disoproxil hemifumarate, tenofovir alafenamide, tenofovir alafenamide hemifumarate, or a combination thereof.

In a particular embodiment, a compound disclosed herein, or a pharmaceutically acceptable salt thereof, is combined with GS-9131, bictegravir, tenofovir, tenofovir disoproxil, tenofovir disoproxil fumarate, tenofovir alafenamide, or tenofovir alafenamide hemifumarate, or a combination thereof.

In a particular embodiment, a compound disclosed herein, or a pharmaceutically acceptable salt thereof, is combined with a first additional therapeutic agent selected from the group consisting of GS-9131, abacavir sulfate, bictegravir, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In a particular embodiment, a compound disclosed herein, or a pharmaceutically acceptable salt thereof, is combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In some embodiments, a compound disclosed herein, or a pharmaceutically acceptable salt thereof, is combined with a capsid inhibitor(s) (e.g., capsid polymerization inhibitors and/or capsid disrupting compounds).

In some embodiments, a compound disclosed herein, or a pharmaceutically acceptable salt thereof, is combined with (about 10 to about 1000 mg) of a capsid inhibitor selected from:

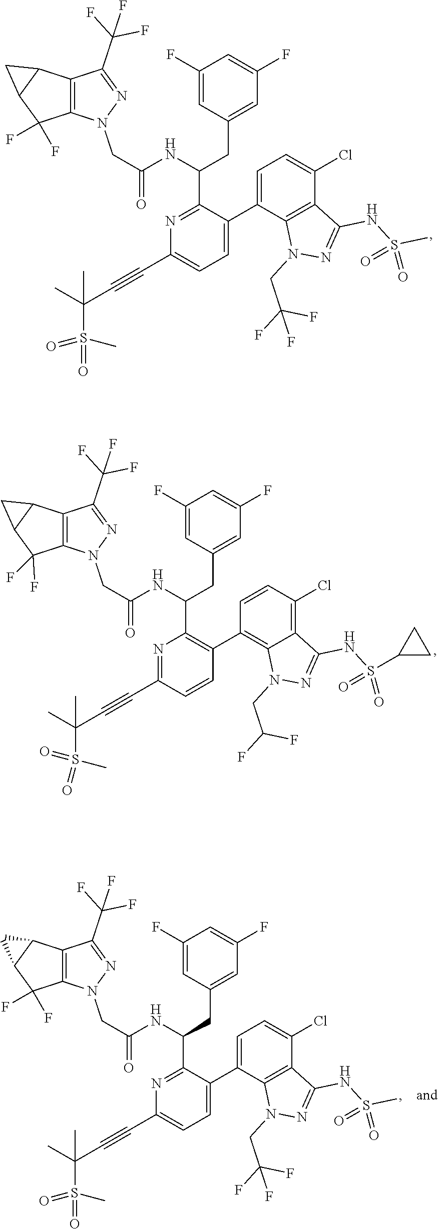 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O ; CC(C)(C#Cc1ccc(-c2ccc(Cl)c3c(NS(=O)(=O)C4CC4)nn(CC(F)F)c23)c(C(Cc2cc(F)cc(F)c2)NC(=O)Cn2nc(C(F)(F)F)c3c2C(F)(F)C2CC32)n1)S(C)(=O)=O ; CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

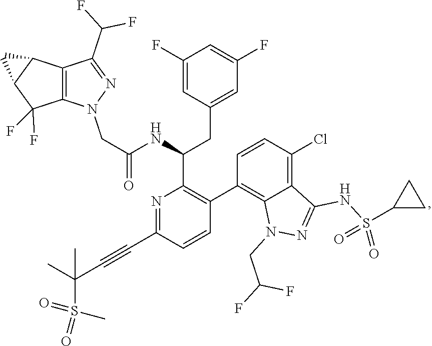 [SMILES: CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

or a pharmaceutically acceptable salt thereof.

In some embodiments, a compound disclosed herein, or a pharmaceutically acceptable salt thereof, is combined with a capsid inhibitor selected from:

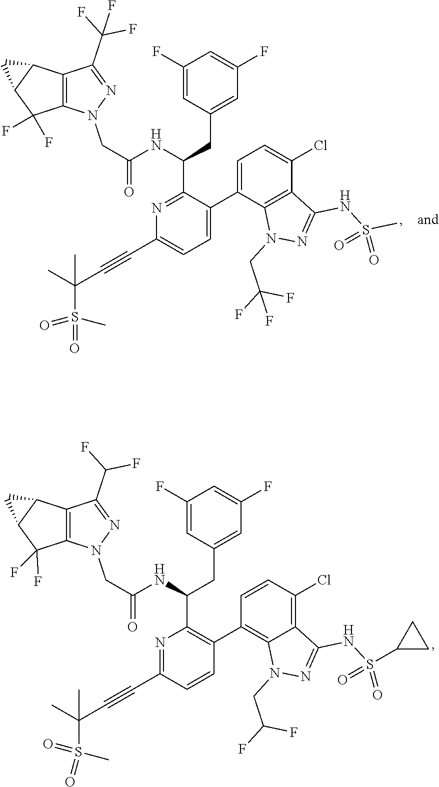 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O ; CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

or a pharmaceutically acceptable salt thereof.

In some embodiments, a compound disclosed herein, or a pharmaceutically acceptable salt thereof, is combined with:

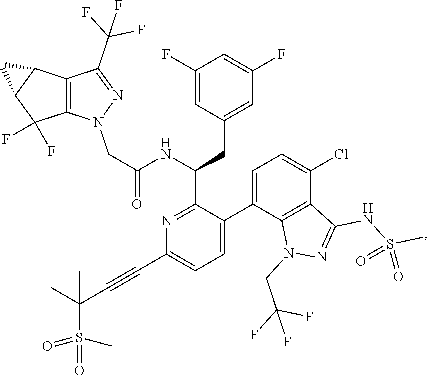 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof.

In some embodiments, a compound disclosed herein, or a pharmaceutically acceptable salt thereof, is combined with:

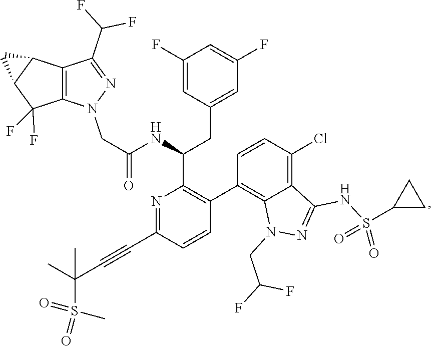 [SMILES: CC(C)(C#Cc1ccc(-c2ccc(Cl)c3c(NS(=O)(=O)C4CC4)nn(CC(F)F)c23)c([C@H](Cc2cc(F)cc(F)c2)NC(=O)Cn2nc(C(F)F)c3c2C(F)(F)[C@@H]2C[C@H]32)n1)S(C)(=O)=O]

or a pharmaceutically acceptable salt thereof.

A compound as disclosed herein may be combined with one or more additional therapeutic agents in any dosage amount of the compound (e.g., from 1 mg to 1000 mg of compound).

In some embodiments, a compound disclosed herein, or a pharmaceutically acceptable salt thereof, is combined with 25-75 mg of bictegravir. In some embodiments, a compound disclosed herein, or a pharmaceutically acceptable salt thereof, is combined with 50 mg of bictegravir (equivalent to 52.5 mg of bictegravir sodium). In some embodiments, a compound disclosed herein, or a pharmaceutically acceptable salt thereof, is combined with 10-70 mg of GS-9131. In some embodiments, a compound disclosed herein, or a pharmaceutically acceptable salt thereof, is combined with 60 mg of GS-9131. In certain embodiments, a compound disclosed herein, or a pharmaceutically acceptable salt thereof, is combined with 5-30 mg tenofovir alafenamide, in the form of tenofovir alafenamide fumarate, tenofovir alafenamide hemifumarate, or tenofovir alafenamide, or any salt of solvate form of tenofovir alafenamide. In certain embodiments, a compound disclosed herein, or a pharmaceutically acceptable salt thereof, is combined with 5-30 mg tenofovir alafenamide fumarate, tenofovir alafenamide hemifumarate, or tenofovir alafenamide, and 200 mg emtricitabine. In certain embodiments, a compound disclosed herein, or a pharmaceutically acceptable salt thereof, is combined with 5-10, 5-15, 5-20, 5-25, 25-30, 20-30, 15-30, or 10-30 mg tenofovir alafenamide fumarate, tenofovir alafenamide hemifumarate, or tenofovir alafenamide, and 200 mg emtricitabine. In certain embodiments, a compound disclosed herein, or a pharmaceutically acceptable salt thereof, is combined with 10 mg tenofovir alafenamide fumarate, tenofovir alafenamide hemifumarate, or tenofovir alafenamide, and 200 mg emtricitabine. In certain embodiments, a compound disclosed herein, or a pharmaceutically acceptable salt thereof, is combined with 25 mg tenofovir alafenamide fumarate, tenofovir alafenamide hemifumarate, or tenofovir alafenamide, and 200 mg emtricitabine. A compound as disclosed herein may be combined with the agents provided herein in any dosage amount of the compound (e.g., from 1 mg to 1000 mg of compound) the same as if each combination of dosages were specifically and individually listed.

In certain embodiments, a compound disclosed herein, or a pharmaceutically acceptable salt thereof, is combined with 200-400 mg tenofovir disoproxil fumarate, tenofovir disoproxil hemifumarate, or tenofovir disoproxil, and 200 mg emtricitabine. In certain embodiments, a compound disclosed herein, or a pharmaceutically acceptable salt thereof, is combined with 200-250, 200-300, 200-350, 250-350, 250-400, 350-400, 300-400, or 250-400 mg tenofovir disoproxil fumarate, tenofovir disoproxil hemifumarate, or tenofovir disoproxil, and 200 mg emtricitabine. In certain embodiments, a compound disclosed herein, or a pharmaceutically acceptable salt thereof, is combined with 300 mg tenofovir disoproxil fumarate, tenofovir disoproxil hemifumarate, or tenofovir disoproxil, and 200 mg emtricitabine. A compound as disclosed herein may be combined with the agents provided herein in any dosage amount of the compound (e.g., from 1 mg to 1000 mg of compound) the same as if each combination of dosages were specifically and individually listed.

In certain embodiments, a compound disclosed herein, or a pharmaceutically acceptable salt thereof, is combined with a HIV nucleoside or nucleotide inhibitor and an integrase inhibitor. In certain embodiments, a compound disclosed herein, or a pharmaceutically acceptable salt thereof, is combined with GS-9131 and bictegravir.

In one embodiment, kits comprising a compound disclosed herein, or a pharmaceutically acceptable salt thereof, in combination with one or more (e.g., one, two, three, one or two, or one to three) additional therapeutic agents are provided.

### Birth Control (Contraceptive) Combination Therapy

Therapeutic agents used for birth control (contraceptive) include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

Gene Therapy and Cell Therapy including the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection.

Examples of dendritic cell therapy include AGS-004.

### Gene Editors

The genome editing system is selected from the group consisting of: a CRISPR/Cas9 system, a zinc finger nuclease system, a TALEN system, a homing endonucleases system, and a meganuclease system.

Examples of HIV targeting CRISPR/Cas9 systems include EBT101.

### CAR-T Cell Therapy

A population of immune effector cells engineered to express a chimeric antigen receptor (CAR), wherein the CAR comprises an HIV antigen-binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T cell or an NK cell. In some embodiments, the T cell is a CD4+ T cell, a CD8+ T cell, or a combination thereof.

Examples of HIV CAR-T include VC-CAR-T.

### TCR-T Cell Therapy

TCR-T cells are engineered to target HIV derived peptides present on the surface of virus-infected cells.

Certain embodiments of the methods disclosed herein exclude the administration of a pharmacokinetic enhancer. For example, in certain methods disclosed herein, the subject is not administered a pharmacokinetic enhancer, such as cobicistat or ritonavir, during the treatment with a compound disclosed herein, or a pharmaceutically acceptable salt thereof. Thus, in certain embodiments, a method of treating or preventing a human immunodeficiency virus (HIV) infection is provided, comprising administering a therapeutically effective amount of a compound disclosed herein, or a pharmaceutically acceptable salt thereof, to a subject in need thereof, wherein the treatment does not comprise administration of a pharmacokinetic enhancer. In certain embodiments, a method of treating or preventing a human immunodeficiency virus (HIV) infection is provided, comprising administering a therapeutically effective amount of a compound disclosed herein, or a pharmaceutically acceptable salt thereof, once daily to a subject in need thereof, wherein the treatment does not comprise administration of a pharmacokinetic enhancer.

The present disclosure also provides all of the P, S, A and I intermediates described in the Examples section below.

### EXAMPLES

Methods for preparing the novel compounds described herein will be apparent to those of skill in the art with suitable procedures being described, for example, in the reaction schemes and examples below.

Section 1 shows preparation of Intermediates as used herein. Section 2 provides example syntheses and compounds. Section 3 shows biological activity.

### 1. Preparation of Intermediates

### 1.1 Synthesis of Intermediate B

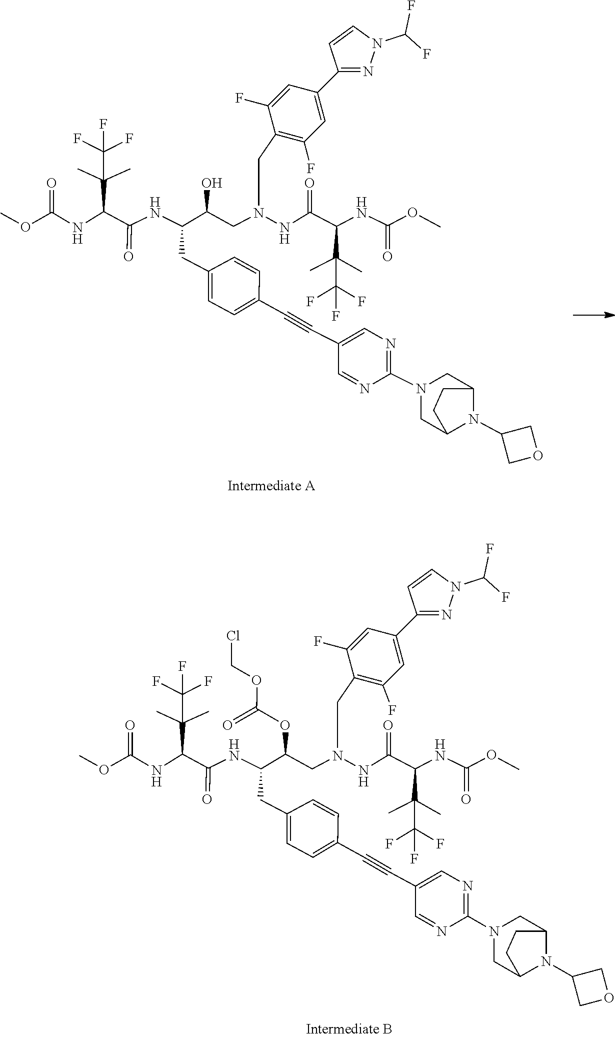 [SMILES: COC(=O)N[C@H](C(=O)N[C@@H](Cc1ccc(C#Cc2cnc(N3CC4CCC(C3)N4C3COC3)nc2)cc1)[C@@H](O)CN(Cc1c(F)cc(-c2ccn(C(F)F)n2)cc1F)NC(=O)[C@@H](NC(=O)OC)C(C)(C)C(F)(F)F)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCCl)C(C)(C)C(F)(F)F]

Synthesis of methyl ((5S,8S,9S,14S)-9-(((chloromethoxy)carbonyl)oxy)-11-(4-(1-(difluoromethyl)-1H-pyrazol-3-yl)-2,6-difluorobenzyl)-16,16,16-trifluoro-15,15-dimethyl-8-(4-((2-(8-(oxetan-3-yl)-3,8-diazabicyclo[3.2.1]octan-3-yl)pyrimidin-5-yl)ethynyl)benzyl)-3,6,13-trioxo-5-(1,1,1-trifluoro-2-methylpropan-2-yl)-2-oxa-4,7,11,12-tetraazahexadecan-14-yl)carbamate (Intermediate B): To a solution of Intermediate A (1.32 mmol) in DCM (3 mL) was added pyridine (13.2 mmol). The solution was cooled to 0° C. in an ice bath, then chloromethyl chloroformate (5.29 mmol) was added slowly dropwise. The reaction mixture was stirred for 90 minutes and allowed to slowly warm to rt. Upon completion of the reaction, the mixture was quenched with sat. ammonium chloride (3 mL) and organics separated. The organic layer was washed with sat. sodium carbonate (3 mL×2), dried over sodium sulfate, filtered, concentrated, and purified by silica gel chromatography, providing Intermediate B. MS (m/z): 1249.6 [M+H]⁺. ¹H NMR (400 MHz, Methanol-d4) δ 8.42 (s, 2H), 8.10 (d, J=2.8 Hz, 1H), 7.69-7.37 (m), 7.33 (d, J=7.9 Hz, 2H), 7.21 (d, J=8.1 Hz, 2H), 6.94 (d, J=2.7 Hz, 1H), 5.92 (d, J=6.4 Hz, 1H), 5.79 (d, J=6.5 Hz, 1H), 4.94-4.87 (m), 4.76 (t, J=6.4 Hz, 2H), 4.66-4.54 (m), 4.44 (s, 1H), 4.40-4.24 (m), 4.06 (d, J=13.2 Hz, 1H), 3.82-3.73 (m), 3.73-3.63 (m), 3.29-3.22 (m), 3.22-3.14 (m), 3.07-2.88 (m), 2.78-2.66 (m), 1.97-1.84 (m), 1.82-1.69 (m), 1.70-1.48 (m), 1.41-1.25 (m), 1.24-1.01 (m), 0.98 (d, J=6.6 Hz, 1H), 0.94-0.82 (m). ¹⁹F NMR (376 MHz, Methanol-d4) δ −77.54 (s), −77.68 (s), −96.87 (d, J=6.2 Hz), −97.03 (d, J=6.1 Hz), −114.68 (s).

### 1.2 Synthesis of Intermediate C

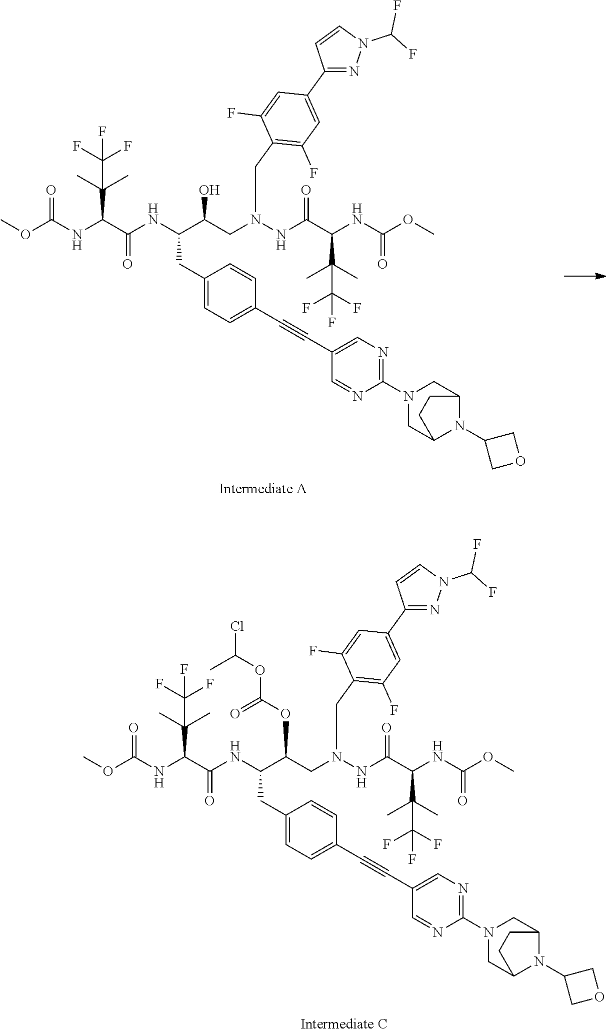 [SMILES: COC(=O)N[C@H](C(=O)N[C@@H](Cc1ccc(C#Cc2cnc(N3CC4CCC(C3)N4C3COC3)nc2)cc1)[C@@H](O)CN(Cc1c(F)cc(-c2ccn(C(F)F)n2)cc1F)NC(=O)[C@@H](NC(=O)OC)C(C)(C)C(F)(F)F)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OC(C)Cl)C(C)(C)C(F)(F)F]

Synthesis of methyl ((5S,8S,9S,14S)-9-(((1-chloroethoxy)carbonyl)oxy)-11-(4-(1-(difluoromethyl)-1H-pyrazol-3-yl)-2,6-difluorobenzyl)-16,16,16-trifluoro-15,15-dimethyl-8-(4-((2-(8-(oxetan-3-yl)-3,8-diazabicyclo[3.2.1]octan-3-yl)pyrimidin-5-yl)ethynyl)benzyl)-3,6,13-trioxo-5-(1,1,1-trifluoro-2-methylpropan-2-yl)-2-oxa-4,7,11,12-tetraazahexadecan-14-yl)carbamate (Intermediate C): To a solution of Intermediate A (1.76 mmol) in DCM (6 mL) was added pyridine (17.6 mmol) at 5° C. 1-chloroethyl carbonochloridate (7.06 mmol) was added slowly dropwise. The reaction mixture was stirred for 90 minutes and allowed to slowly warm to rt. Upon completion of the reaction, the mixture was quenched with sat. ammonium chloride (6 mL) and organics separated. The organic layer was washed with sat. sodium carbonate (6 mL×2), dried over sodium sulfate, filtered, concentrated, and purified by silica gel chromatography, providing Intermediate C. MS (m/z): 1263.2 [M+H]⁺. ¹H NMR (400 MHz, Methanol-d4) δ 8.42 (s, 2H), 8.10 (dd, J=2.7, 1.7 Hz, 1H), 7.71-7.36 (m), 7.36-7.29 (m), 7.21 (t, J=7.7 Hz, 2H), 6.94 (t, J=2.9 Hz, 1H), 6.57-6.45 (m), 4.96-4.87 (m), 4.76 (t, J=6.4 Hz, 2H), 4.72-4.63 (m), 4.58 (t, J=5.8 Hz, 2H), 4.56-4.49 (m), 4.45 (s, 1H), 4.39-4.23 (m), 4.05 (d, J=13.2 Hz, 1H), 3.81-3.72 (m), 3.72-3.63 (m), 3.26 (s, 3H), 3.22-3.12 (m), 3.09-2.86 (m), 2.79-2.65 (m), 1.95-1.88 (m), 1.84 (dd, J=13.4, 5.8 Hz, 3H), 1.65-1.54 (m), 1.27-0.99 (m). ¹⁹F NMR (376 MHz, Methanol-d4) δ −77.49 (d, J=13.1 Hz), −77.68 (d, J=20.3 Hz), −96.87 (dd, J=10.1, 5.4 Hz), −97.03 (dd, J=10.2, 5.3 Hz), −114.64 (d, J=8.9 Hz). 1.3 Synthesis of Intermediate D

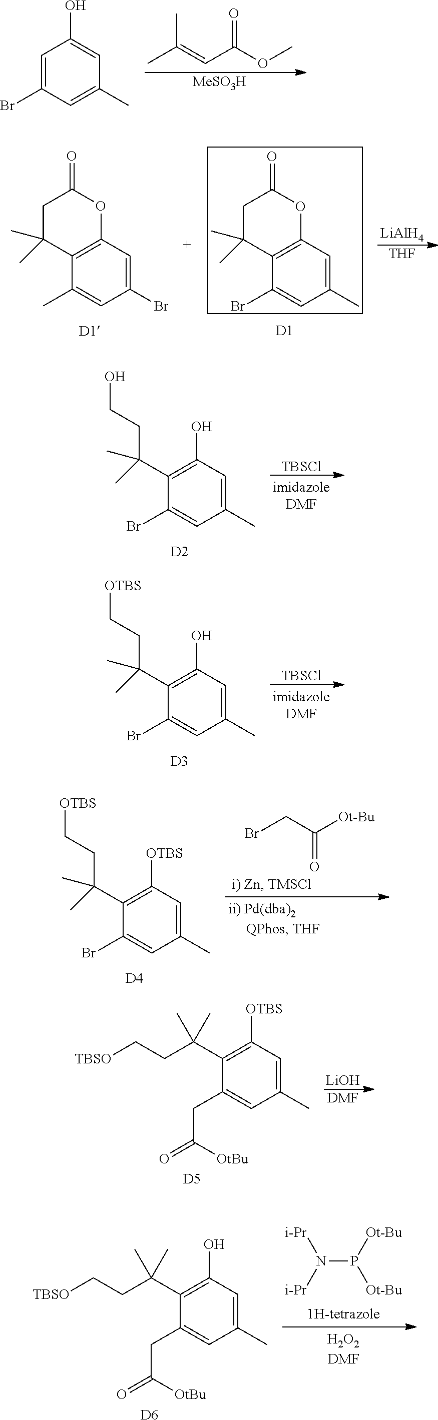

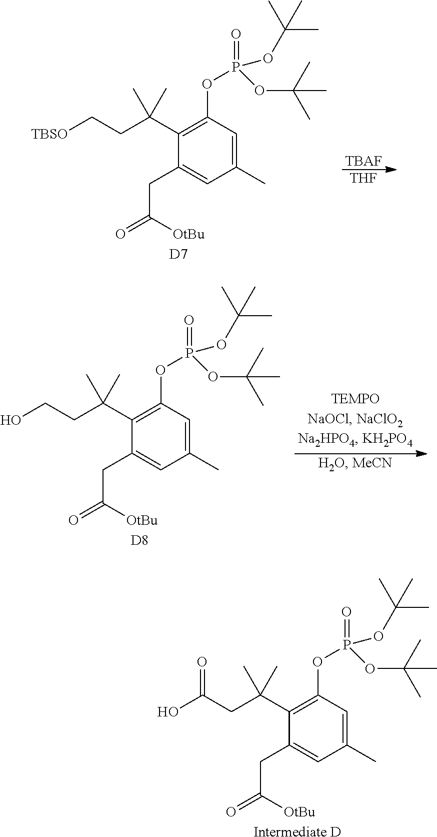

Synthesis of 5-Bromo-4,4,7-trimethylchroman-2-one (D1): A flask was charged with 3-bromo-5-methylphenol (26.7 mmol) and methanesulfonic acid (53.5 mmol) under nitrogen. Methyl 3-methylbut-2-enoate (29.4 mmol) was added, the flask was fitted with a reflux condenser and the reaction was heated at 90° C. for 30 minutes, 120° C. for 30 minutes and 150° C. for 3 hours. The reaction was cooled to RT, placed into an ice-water bath and diluted with water (150 mL). The aqueous layer was extracted with EtOAc (2×200 mL), washed with sat. NaHCO₃ (2×100 mL), washed with brine, dried over MgSO₄, filtered and concentrated under reduced pressure. The residue was purified by silica gel chromatography to afford title compound D1. MS (m/z) 269.02/270.95 [M+H]⁺.

Synthesis of 3-Bromo-2-(4-hydroxy-2-methylbutan-2-yl)-5-methylphenol (D2): A flask was charged with a solution of LAH (2.0M in THF, 8.17 mmol) and THE (10 mL). The solution was cooled to 0° C. A solution of D1 (7.43 mmol) in THE (20 mL) was added dropwise. The ice bath was removed and the reaction was stirred at RT for 2.5 hours. The reaction was cooled to 5° C. Water (310 μL) was added slowly, followed by 15% NaOH_{(aq)} (310 μL), then water again (930 μL). The mixture was warmed to RT and stirred overnight. The mixture was diluted with EtOAc, MgSO₄ was added and the mixture was filtered. The filter cake was rinsed with EtOAc. The crude product was purified by silica gel chromatography to afford title compound D2. MS (m/z) 271.15/273.03 [M−H]⁻.

Synthesis of 3-Bromo-2-(4-((tert-butyldimethylsilyl)oxy)-2-methylbutan-2-yl)-5-methylphenol (D3): A flask was charged with D2 (6.55 mmol), DMF (15.0 mL) and imidazole (16.4 mmol) under nitrogen. The solution was cooled to 0° C., TBSCl (8.51 mmol) was added in one portion, the ice bath was removed and the reaction was stirred at RT for 2 hours and then cooled to 0° C. The reaction was diluted with water (75 mL), extracted with EtOAc (3×), washed with 5% LiCl_{(aq)}, dried over MgSO₄, filtered and concentrated. The crude product was purified by silica gel chromatography to afford title compound D3. MS (m/z) 385.31/386.9 [M−H]⁻.

Synthesis of (3-Bromo-2-(4-((tert-butyldimethylsilyl)oxy)-2-methylbutan-2-yl)-5-methylphenoxy)(tert-butyl)dimethylsilane (D4): A flask was charged with D3 (2.55 mmol), DMF (12.0 mL) and imidazole (6.37 mmol) under nitrogen. TBSCl (3.31 mmol) was added in one portion and the flask was fitted with a reflux condenser. The reaction was heated at 65° C. for 8 hours. The reaction was concentrated, diluted with water, extracted with EtOAc (3×), washed with 5% LiCl_{(aq)}, dried over MgSO₄, filtered and concentrated. The mixture was purified by silica gel chromatography to afford title compound D4. ¹H NMR (400 MHz, Chloroform-d) δ 7.04 (d, J=1.6 Hz, 1H), 6.53 (d, J=1.8 Hz, 1H), 3.52-3.43 (m, 2H), 2.29-2.20 (m, 2H), 2.17 (s, 3H), 1.60 (s, 6H), 1.00 (s, 9H), 0.83 (s, 9H), 0.29 (s, 6H), 0.03 (s, 6H) ppm.

Synthesis of tert-butyl 2-(3-((tert-butyldimethylsilyl)oxy)-2-(4-((tert-butyldimethylsilyl)oxy)-2-methylbutan-2-yl)-5-methylphenyl)acetate (D5): A flask was charged with zinc dust (<10 μm, 9.00 mmol) and THE (3.6 mL) under nitrogen. The flask was fitted with an internal temperature probe. Chlorotrimethylsilane (0.60 mmol) was added and the mixture was stirred for 15 minutes (a 1-2° C. exotherm was recorded). A solution of tert-butyl 2-bromoacetate (6.00 mmol) in THE (2.66 mL) was added cautiously. The mixture was stirred until it cooled back to RT and then stirring was stopped. The supernatant was titrated using I₂ in a 0.5M solution of LiCl in THE. The concentration of the 2-tert-butoxy-2-oxoethylzinc bromide organozinc reagent was determined to be 0.63M. A separate flask was charged with D4 (1.35 mmol), Pd(dba)₂ (0.068 mmol) and QPhos (0.068 mmol). The flask was purged with nitrogen. THE (4.7 mL) was added, followed by 2-tert-butoxy-2-oxoethylzinc bromide (1.62 mmol). The flask was fitted with a reflux condenser and the reaction was heated at 55° C. for 15-30 minutes. The reaction was cooled to RT, quenched with sat. NH₄Cl_{(aq)}, extracted with EtOAc (3×), washed with brine, dried over MgSO₄, filtered and concentrated. The crude product was purified by silica gel chromatography to afford title compound D5. MS (m/z) 559.13 [M+Na]⁺.

Synthesis of tert-butyl 2-(2-(4-((tert-butyldimethylsilyl)oxy)-2-methylbutan-2-yl)-3-hydroxy-5-methylphenyl)acetate (D6): To a solution of D5 (5.14 mmol) in DMF (4.0 mL) was added LiOH (5.14 mmol) under nitrogen. The mixture was stirred at RT for 18 hours. The reaction was diluted with water, extracted with EtOAc (3×), the combined organic layers were sequentially washed with 5% aqueous LiCl and brine, then dried over MgSO₄, filtered and concentrated under reduced pressure. The residue was purified by silica gel chromatography to afford title compound D6. MS (m/z) 421.16 [M−H]⁻.

Synthesis of tert-butyl 2-(2-(4-((tert-butyldimethylsilyl)oxy)-2-methylbutan-2-yl)-3-((di-tert-butoxyphosphoryl)oxy)-5-methylphenyl)acetate (D7): To a solution of D6 (0.821 mmol) in DMF (3.0 mL) was added di-tert-butyl N,N-diisopropylphosphoramidite (2.46 mmol) followed by 1H-tetrazole (2.87 mmol). The solution was heated at 50° C. for 5 hours. The reaction was cooled to 0° C., 30% H₂O_{2(aq)} (3.28 mmol) was added, and the reaction was stirred for 1 hour then allowed to warm to RT. The reaction was diluted with water and extracted with 25% EtOAc/hexanes (3×). The combined organic layers were washed with 5% LiCl_{(aq)}, then sat. aqueous Na₂S₂O₃, dried over MgSO₄, filtered and concentrated. The residue was purified by silica gel chromatography to yield title compound D7. MS (m/z) 637.05 [M+Na]⁺.

Synthesis of tert-butyl 2-(3-((di-tert-butoxyphosphoryl)oxy)-2-(4-hydroxy-2-methylbutan-2-yl)-5-methylphenyl)acetate (D8): A solution of D7 (0.569 mmol) in THE (5.0 mL) was cooled to 0° C. TBAF (1.0 M in THF, 1.14 mmol) was added and the reaction was stirred for 18 hours, gradually warming up to RT. The reaction was concentrated and diluted with EtOAc. The organic layer was washed with water (2×) and brine, dried over MgSO₄, then filtered and concentrated under reduced pressure. The residue was purified by silica gel chromatography to yield title compound D8. MS (m/z) 522.95 [M+Na]⁺.

Synthesis of 3-(2-(2-(Tert-butoxy)-2-oxoethyl)-6-((di-tert-butoxyphosphoryl)oxy)-4-methylphenyl)-3-methylbutanoic acid (Intermediate D): To a solution of D8 (0.356 mmol) in ACN/water (1:1, 3.0 mL) was added TEMPO (0.018 mmol), potassium dihydrogen phosphate (0.178 mmol) and disodium hydrogen phosphate (0.178 mmol). The solution was cooled to 0° C. Sodium chlorite (0.533 mmol) was added, followed by sodium hypochlorite (8.25% NaOCl, 266 μL). The ice bath was removed and the reaction was stirred for 3.5 hours. The reaction was diluted with water, extracted with EtOAc (3×), the combined organic layers were washed with brine, dried over MgSO₄, filtered and concentrated under reduced pressure to yield title compound Intermediate D which was used without further purification. MS (m/z) 536.85 [M+Na]⁺.

### Example 1.4 Synthesis of Intermediate E

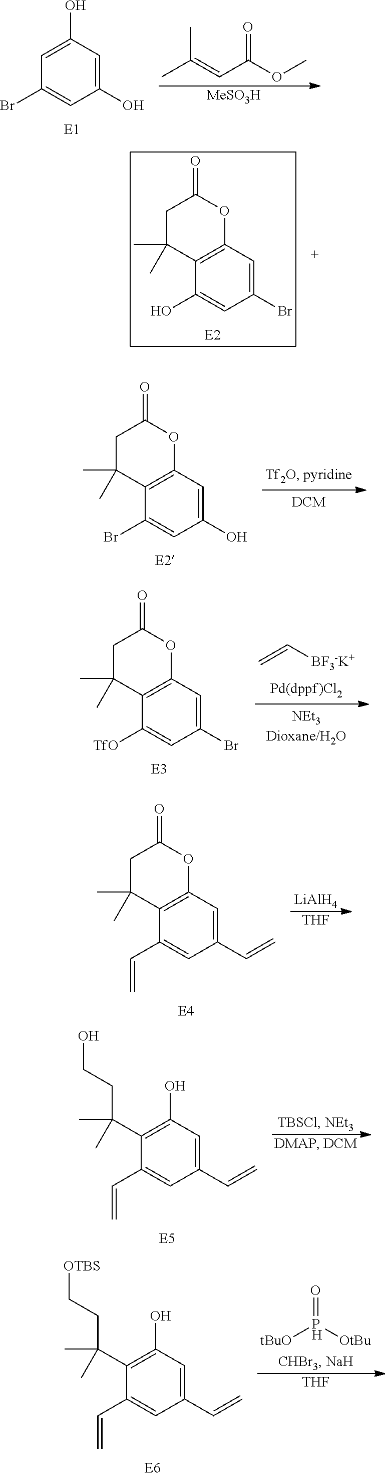

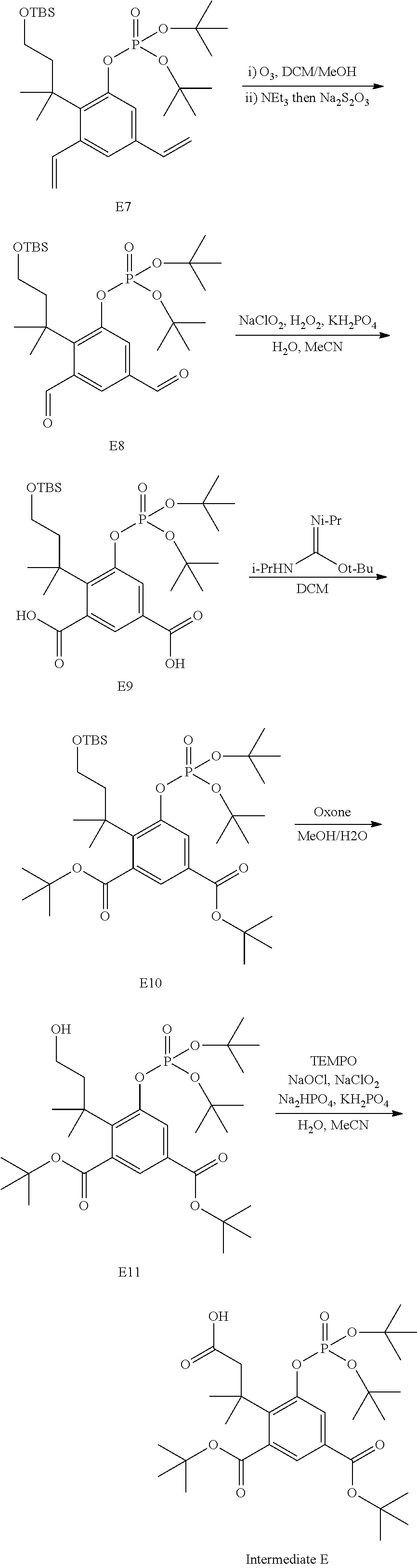

Synthesis of 7-bromo-5-hydroxy-4,4-dimethyl-chroman-2-one (E2): A mixture of 5-bromobenzene-1,3-diol (E1, 265 mmol), methyl 3-methylbut-2-enoate (291 mmol) and MsOH (529 mmol) was stirred at 80° C. for 12 hours. The reaction mixture was diluted with EtOAc (300 mL) and water (100 mL), then the organic layer was washed with aq. sodium bicarbonate (300 mL). The organic layer was dried over anhydrous sodium sulfate, filtered, and concentrated under reduced pressure. The residue was purified by silica gel chromatography to yield 7-bromo-5-hydroxy-4,4-dimethyl-chroman-2-one (E2) and 5-Bromo-7-hydroxy-4,4-dimethylchroman-2-one E2′. E2: MS (m/z): 271.0 [M+H]⁺. H NMR (400 MHz, Chloroform-d) δ 6.83 (d, J=1.9 Hz, 1H), 6.69 (d, J=2.0 Hz, 1H), 5.27 (s, 1H), 2.61 (s, 2H), 1.46 (s, 6H). E2′: MS (m/z): 271.0 [M+H]⁺. ¹H NMR (400 MHz, Chloroform-d) δ 6.93 (d, J=2.6 Hz, 1H), 6.59 (t, J=2.5 Hz, 1H), 5.33 (s, 1H), 2.64 (s, 2H), 1.55 (s, 6H).

Synthesis of 5-bromo-4,4-dimethyl-2-oxochroman-7-yl trifluoromethanesulfonate (E3): To a solution of 7-bromo-5-hydroxy-4,4-dimethyl-chroman-2-one E2 (18.3 mmol) in DCM (73.2 mL) was added pyridine (36.6 mmol) and Tf₂O (18.3 mmol) at 0° C. The mixture was stirred at rt for 12 hours. The reaction mixture was poured into water (70 mL) and extracted with DCM (70 mL×3). The combined organic layers were dried over sodium sulfate, filtered, and concentrated under reduced pressure. The residue was purified by silica gel chromatography to yield 7-bromo-4,4-dimethyl-2-oxochroman-5-yl trifluoromethanesulfonate E3. MS (m/z): 404.9 [M+H]⁺.

Synthesis of 4,4-dimethyl-5,7-divinylchroman-2-one (E4): To a mixture of 7-bromo-4,4-dimethyl-2-oxochroman-5-yl trifluoromethanesulfonate E3 (7.49 mmol), potassium trifluoro(vinyl)borate (22.5 mmol) and Pd(dppf)Cl₂ (0.75 mmol) was added 1,4-dioxane (41.6 mL), n-BuOH (20.7 mL) and NEt₃ (22.5 mmol) under N₂. The mixture was stirred at 80° C. for 12 hours under N₂. At this point the reaction stalled, so the reaction mixture was cooled to rt, and sodium carbonate (22.5 mmol), Pd(dppf)Cl₂ (0.75 mmol), dioxane (20 mL), and water (10 mL) were added, then the reaction was heated to 100° C. for 12 hours under N₂. The reaction was then concentrated under reduced pressure, diluted with water (100 mL) and extracted with EtOAc (100 mL×3). The combined organic layers were dried over sodium sulfate, filtered, and concentrated under reduced pressure. The residue was purified by silica gel chromatography to yield 4,4-dimethyl-5,7-divinylchroman-2-one E4. MS (m/z): 228.9 [M+H]⁺. ¹H NMR (400 MHz, Chloroform-d) δ 7.17-6.99 (m, 3H), 6.65 (dd, J=17.6, 10.8 Hz, 1H), 5.76 (dd, J=17.6, 0.7 Hz, 1H), 5.49 (dd, J=17.2, 1.5 Hz, 1H), 5.38-5.27 (m, 2H), 2.59 (s, 2H), 1.44 (s, 6H).

Synthesis of 2-(4-hydroxy-2-methylbutan-2-yl)-3,5-divinylphenol (E5): To a 1M solution of LAH (6.78 mmol) was added a solution of 4,4-dimethyl-5,7-divinylchroman-2-one E4 (3.39 mmol) in THE (4 mL) at 0° C. The mixture was stirred at 0° C. for 2 hours. Water (2 mL) and MgSO₄ (300 mg) were added to the mixture. The reaction mixture was filtered and the filter cake was washed with THE (5 mL×3). The filtrate was dried over sodium sulfate, filtered, and concentrated under reduced pressure. The residue was purified by silica gel chromatography to yield 2-(4-hydroxy-2-methylbutan-2-yl)-3,5-divinylphenol E5. MS (m/z): 231.2 [M−H]⁻. ¹H NMR (400 MHz, Chloroform-d) δ 7.21 (dd, J=17.1, 10.8 Hz, 1H), 6.81 (d, J=2.0 Hz, 1H), 6.68 (d, J=2.0 Hz, 1H), 6.57 (dd, J=17.6, 10.8 Hz, 1H), 5.67 (dt, J=17.5, 1.1 Hz, 1H), 5.32-5.12 (m, 3H), 3.60 (t, J=7.1, 1.3 Hz, 2H), 2.21 (t, J=7.1, 2.3 Hz, 2H), 1.54 (s, 6H).

Synthesis of 2-(4-((tert-butyldimethylsilyl)oxy)-2-methylbutan-2-yl)-3,5-divinylphenol (E6): To a solution of 2-(4-hydroxy-2-methylbutan-2-yl)-3,5-divinylphenol E5 (2.95 mmol) in DCM (29.5 mL) was added NEt₃ (4.42 mmol) and TBSCl (3.54 mmol), followed by DMAP (1.47 mmol). The mixture was stirred at rt for 2 hours. The reaction mixture was poured into water (30 mL) and extracted with DCM (30 mL×3). The combined organic layers were dried over sodium sulfate, filtered, and concentrated under reduced pressure. The residue was purified by silica gel chromatography to yield 2-(4-((tert-butyldimethylsilyl)oxy)-2-methylbutan-2-yl)-3,5-divinylphenol E6. MS (m/z): 345.2 [M−H]⁻. ¹H NMR (400 MHz, Chloroform-d) δ 7.16 (dd, J=17.1, 10.8 Hz, 1H), 6.83 (d, J=2.0 Hz, 1H), 6.76 (d, J=2.0 Hz, 1H), 6.59 (dd, J=17.6, 10.9 Hz, 1H), 5.97 (s, 1H), 5.69 (dd, J=17.5, 0.9 Hz, 1H), 5.27 (dd, J=17.2, 1.9 Hz, 1H), 5.23-5.17 (m, 1H), 5.14 (dd, J=10.8, 1.8 Hz, 1H), 3.61 (t, J=6.6 Hz, 2H), 2.09 (t, J=6.7 Hz, 2H), 1.53 (s, 6H), 0.87 (s, 9H), 0.01 (s, 6H).

Synthesis of di-tert-butyl (2-(4-((tert-butyldimethylsilyl)oxy)-2-methylbutan-2-yl)-3,5-divinylphenyl) phosphate (E7): To a solution of 2-(4-((tert-butyldimethylsilyl)oxy)-2-methylbutan-2-yl)-3,5-divinylphenol E6 (2.47 mmol) in THE (6.18 mL) was added di-tert-butyl phosphite (3.31 mmol) and bromoform (3.31 mmol) at 0° C. The solution was stirred for 5 min, then sodium hydride (3.31 mmol) was added. The reaction was stirred at rt for 2 hours, then quenched with water (6 mL). The mixture was extracted with EtOAc (6 mL×3), and the combined organics were dried over sodium sulfate, filtered, and concentrated under reduced pressure. The residue was purified by silica gel chromatography to yield di-tert-butyl (2-(4-((tert-butyldimethylsilyl)oxy)-2-methylbutan-2-yl)-3,5-divinylphenyl) phosphate E7. MS (m/z): 561.0 [M+Na]⁺.

Synthesis of di-tert-butyl (2-(4-((tert-butyldimethylsilyl)oxy)-2-methylbutan-2-yl)-3,5-diformylphenyl) phosphate (E8): To a solution of di-tert-butyl (2-(4-((tert-butyldimethylsilyl)oxy)-2-methylbutan-2-yl)-3,5-divinylphenyl) phosphate E7 (2.13 mmol) in DCM/MeOH (3:1, 80.0 mL) was carefully bubbled O₃ (g) at −78° C. When full conversion was observed, the reaction was sparged with 02 (g) for 5 mins, after which NEt₃ (17.1 mmol) was carefully added. The resulting mixture was warmed to rt, then 50 mL of saturated Na₂S₂O₃ (aq) was added. The reaction was diluted with water and extracted with DCM (80 mL×3). The combined organic layers were washed with brine, dried over sodium sulfate, filtered, and concentrated under reduced pressure. The residue was purified by silica gel chromatography to yield di-tert-butyl (2-(4-((tert-butyldimethylsilyl)oxy)-2-methylbutan-2-yl)-3,5-diformylphenyl) phosphate E8. MS (m/z): 564.8 [M+Na]⁺.

Synthesis of 4-(4-((tert-butyldimethylsilyl)oxy)-2-methylbutan-2-yl)-5-((di-tert-butoxyphosphoryl)oxy)isophthalic acid (E9): To a solution of di-tert-butyl (2-(4-((tert-butyldimethylsilyl)oxy)-2-methylbutan-2-yl)-3,5-diformylphenyl) phosphate E8 (1.35 mmol), potassium dihydrogen phosphate (1.08 mmol) and hydrogen peroxide (4.06 mmol, 30 wt. % in water) in ACN/water (1:1, 13.0 mL) at 0° C. was added sodium chlorite (5.41 mmol). The reaction was warmed to rt and stirred for 2 hours, then quenched with aqueous sodium sulfite, adjusted to pH 2 with aqueous 1N HCl, and extracted with DCM (13 mL×3). The combined organics were dried over sodium sulfate, filtered, and concentrated under reduced pressure to yield 4-(4-((tert-butyldimethylsilyl)oxy)-2-methylbutan-2-yl)-5-((di-tert-butoxyphosphoryl)oxy)isophthalic acid E9 which was used without further purification. MS (m/z): 596.8 [M+Na]⁺.

Synthesis of di-tert-butyl 4-(4-((tert-butyldimethylsilyl)oxy)-2-methylbutan-2-yl)-5-((di-tert-butoxyphosphoryl)oxy)isophthalate (E10): To a solution of 4-(4-((tert-butyldimethylsilyl)oxy)-2-methylbutan-2-yl)-5-((di-tert-butoxyphosphoryl)oxy)isophthalic acid E9 (0.762 mmol) in DCM (6.0 mL) was slowly added 2-tert-butyl-1,3-diisopropylurea (7.62 mmol). The resulting mixture was stirred at room temperature for 18 hours. Additional 2-tert-butyl-1,3-diisopropylurea (3.81 mmol) was added, and the reaction was heated to 40° C. for 8 hours, then cooled to rt. The reaction mixture was diluted with dichloromethane and filtered over Celite®. The filtrate was concentrated and used without further purification to yield di-tert-butyl 4-(4-((tert-butyldimethylsilyl)oxy)-2-methylbutan-2-yl)-5-((di-tert-butoxyphosphoryl)oxy)isophthalate E10. MS (m/z): 709.0 [M+Na]⁺.

Synthesis of di-tert-butyl 5-((di-tert-butoxyphosphoryl)oxy)-4-(4-hydroxy-2-methylbutan-2-yl)isophthalate (E11): A flask charged with di-tert-butyl 4-(4-((tert-butyldimethylsilyl)oxy)-2-methylbutan-2-yl)-5-((di-tert-butoxyphosphoryl)oxy)isophthalate E10 (0.763 mmol), MeOH (2.5 mL), and water (2.5 mL) was cooled to 0° C. Oxone© (0.763 mmol) was added, followed by the addition of MeCN (5.0 mL). The reaction was warmed to rt and stirred for 90 minutes. The reaction was quenched with aqueous sodium sulfite, concentrated, diluted with water and extracted with EtOAc (5 mL×3). The combined organic layers were washed with brine, dried over sodium sulfate, filtered, and concentrated. The crude material was purified by silica gel chromatography to afford di-tert-butyl 5-((di-tert-butoxyphosphoryl)oxy)-4-(4-hydroxy-2-methylbutan-2-yl)isophthalate E11. MS (m/z): 538.9 [(M-tBu)+Na]⁺.

Synthesis of 3-(2,4-bis(tert-butoxycarbonyl)-6-((di-tert-butoxyphosphoryl)oxy)phenyl)-3-methylbutanoic acid (Intermediate E): To a solution of di-tert-butyl 5-((di-tert-butoxyphosphoryl)oxy)-4-(4-hydroxy-2-methylbutan-2-yl)isophthalate E11 (0.148 mmol) in ACN/water (1:1, 2.8 mL) was added TEMPO (0.0074 mmol), potassium dihydrogen phosphate (0.074 mmol) and disodium hydrogen phosphate (0.074 mmol). The solution was cooled to 0° C. Sodium chlorite (0.223 mmol) was added, followed by sodium hypochlorite (8.25% NaOCl (aq), 0.181 mmol). The ice bath was removed and the reaction was stirred for 1 hour. Sodium chlorite (0.074 mmol) was added and the reaction was stirred for an additional 2 hours. The reaction was diluted with water and quenched with aqueous sodium sulfite and extracted with EtOAc (3 mL×3). The combined organic layers were washed with brine, dried over sodium sulfate, filtered, and concentrated under reduced pressure to yield 3-(2,4-bis(tert-butoxycarbonyl)-6-((di-tert-butoxyphosphoryl)oxy)phenyl)-3-methylbutanoic acid Intermediate E which was used without further purification. MS (m/z): 529.4 [(M-tBu)-H]⁻.

### 1.5 Synthesis of Intermediate F

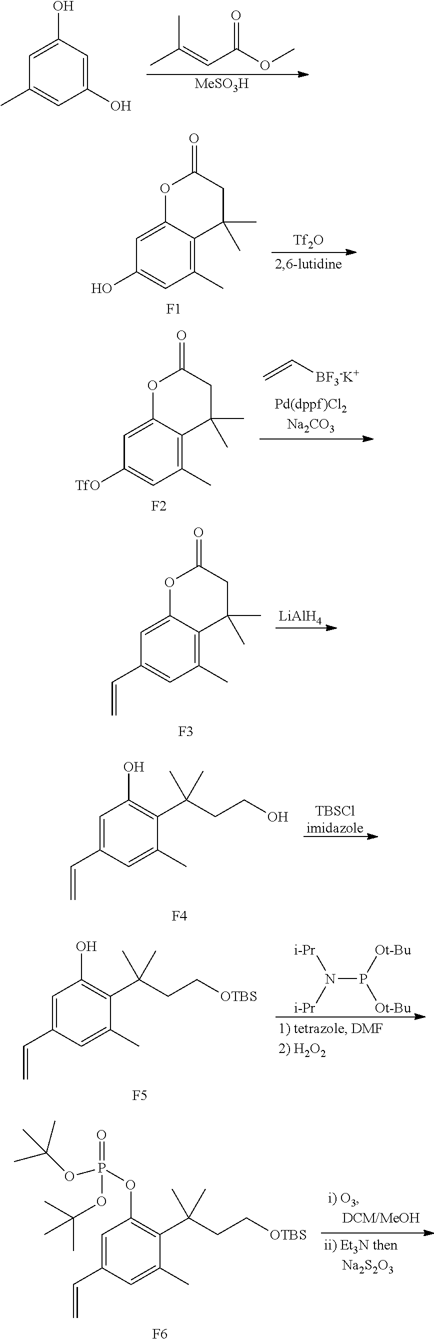

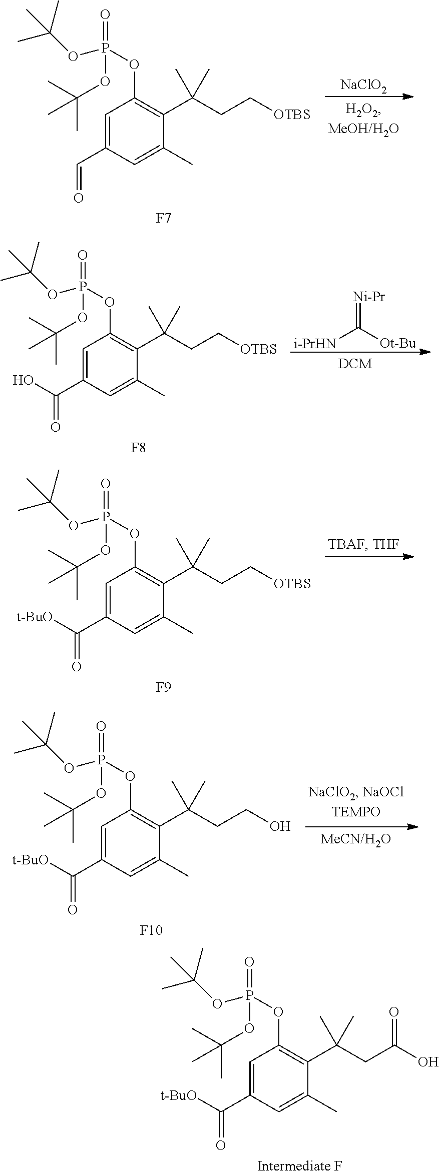

Synthesis of 7-hydroxy-4,4,5-trimethylchroman-2-one (F1): To a stirred solution of 3,5-dihydroxytoluene (483 mmol) in methanesulfonic acid (62.8 mL) was added methyl 3-methylbut-2-enoate (532 mmol). The mixture was stirred at 80° C. for 3 h and monitored by LCMS. After completion, the reaction mixture was diluted with water and extracted with EtOAc. The combined organic layers were dried over anhydrous Na₂SO₄, filtered, and concentrated. The crude mixture was purified by column chromatography (0% to 30% EtOAc in hexanes) to afford title compound F1. MS (m/z): 207.1 [M+H]⁺.

Synthesis of 4,4,5-trimethyl-2-oxochroman-7-yl trifluoromethanesulfonate (F2): To a stirred solution F1 (249 mmol) in dichloromethane (20 mL) at 0° C. was added 2,6-lutidine (274 mmol) followed by triflic anhydride (262 mmol, 1.05 equiv) and the reaction mixture was stirred for 2 h and monitored by LCMS. After completion, the reaction mixture was diluted with dichloromethane, washed with 1M HCl and brine. The combined organic layers were dried over anhydrous Na₂SO₄, filtered, and concentrated. The crude mixture was purified by column chromatography (0% to 10% EtOAc in hexanes) to afford title compound F2. MS (m/z): 339.1 [M+H]⁺.

Synthesis of 4,4,5-trimethyl-7-vinylchroman-2-one (F3): To a stirred solution of F2 (161 mmol) in dioxane (130 mL) and water (15 mL) was added potassium vinyltrifluoroborate (193 mmol), sodium carbonate (483 mmol) and Pd(dppf)Cl₂ (16.1 mmol). The mixture was degassed and backfilled with argon (3×) and heated to 100° C. for 2 h and monitored by LCMS. After completion, the reaction mixture was diluted with EtOAc and washed with sat. aq. solutions of NH₄Cl and brine. The combined organic layers were dried over anhydrous Na₂SO₄, filtered, and concentrated. The crude mixture was purified by column chromatography (0% to 10% EtOAc in hexanes) to afford title compound F3. MS (m/z): 217.2 [M+H]⁺.

Synthesis of 2-(4-hydroxy-2-methylbutan-2-yl)-3-methyl-5-vinylphenol (F4): F3 (83.2 mmol) was dissolved in THE (121 mL), cooled to 0° C. and treated with LiAlH₄ (53 mL, 2 M in THF). The reaction mixture was then gradually warmed to room temperature over 16 h. After completion, the reaction mixture was quenched with ice water, filtered through a pad of Celite®, and extracted with EtOAc. The combined organic layers were dried over anhydrous Na₂SO₄, filtered, and concentrated to yield title compound F4 which was used without purification.

Synthesis of 2-(4-((tert-butyldimethylsilyl)oxy)-2-methylbutan-2-yl)-3-methyl-5-vinylphenol (F5): To a solution of F4 (20.1 mmol) in DMF (30 mL) was added imidazole (50.2 mmol) and TBSCl (26 mmol) sequentially. The reaction mixture was stirred for 2 h and monitored by LCMS. After completion, the reaction mixture was diluted with diethyl ether and washed with water. The combined organic layers were dried over anhydrous Na₂SO₄, filtered, and concentrated. The crude mixture was purified by column chromatography (0% to 10% EtOAc in hexanes) to afford title compound F5. MS (m/z): 358.1 [M+Na]⁺.

Synthesis of di-tert-butyl (2-(4-((tert-butyldimethylsilyl)oxy)-2-methylbutan-2-yl)-3-methyl-5-vinylphenyl) phosphate (F6): To a solution of F5 (18.7 mmol) in DMF (200 mL) was added di-t-butyl-N,N-diisopropylphosphoramidite (56.0 mmol) and 1-H-tetrazole (7.1 mmol) sequentially. The reaction mixture was stirred at room temperature while monitored by TLC and LCMS. After 6 h, the reaction mixture was cooled to 0° C. and treated with aq. H₂O₂ solution (24 mL, 50 wt. %). The reaction was gradually warmed to room temperature and stirred. After completion, the reaction mixture was diluted with EtOAc and washed with sat. aq. sodium thiosulfate solution. The combined organic layers were dried over anhydrous Na₂SO₄, filtered, and concentrated. The crude mixture was purified by column chromatography to afford title compound F6. MS (m/z): 549.2 [M+Na]⁺.

Synthesis of di-tert-butyl (2-(4-((tert-butyldimethylsilyl)oxy)-2-methylbutan-2-yl)-5-formyl-3-methylphenyl) phosphate (F7): To a solution of F6 (26.8 mmol) in 120 mL of a 1:1 mixture of DCM/MeOH was carefully bubbled O₃ gas at −78° C. When full conversion was observed, the reaction was sparged with Ar gas for 5 mins, after which Et₃N (10 mL) was carefully added. To the resulting mixture was then added 200 mL of saturated Na₂S₂O₃ and the reaction was stirred a further 1 h. Upon completion of this time, the contents were transferred to a separatory funnel using DCM (200 mL) and H₂O (200 mL). The aqueous layer was extracted with DCM (2×100 mL) and the organic fractions were collected, dried over Na₂SO₄, concentrated under reduced pressure to yield title compound F7, which was used without further purification. MS (m/z): 551.30 [M+Na]⁺.

Synthesis of 4-(4-((tert-butyldimethylsilyl)oxy)-2-methylbutan-2-yl)-3-((di-tert-butoxyphosphoryl)oxy)-5-methylbenzoic acid (F8): To a solution of F7 (17.9 mmol), KH₂PO₄ (7.15 mmol, 0.4 equiv), hydrogen peroxide (30% aqueous solution, 26.8 mmol, 1.5 equiv) in 75 mL of 1:1 MeCN/H₂O was added a solution of sodium chlorite (35.7 mmol, 2 equiv) at 0° C. and the resulting mixture stirred for 30 mins at this temperature before slowly warming to room temperature. Upon full conversion, the reaction was quenched with saturated sodium sulfite solution and the contents were transferred to a separatory funnel using DCM (200 mL) and H₂O (200 mL) where the pH was carefully adjusted to 2 with 1M HCl solution. The aqueous layer was extracted with DCM (2×100 mL) and the organic fractions were collected, dried over Na₂SO₄, concentrated under reduced pressure to yield title compound F8 which was used without further purification. MS (m/z): 567.3 [M+Na]⁺.

Synthesis of tert-butyl 4-(4-((tert-butyldimethylsilyl)oxy)-2-methylbutan-2-yl)-3-((di-tert-(butoxyphosphoryl)oxy)-5-methylbenzoate (F9): To a solution of F8 (23.3 mmol) in 50 mL of DCM was added 2-tert-butyl-1,3-diisopropylurea (117 mmol, 5 equiv) at rt and the resulting solution left to stir overnight. Upon completion, the reaction was diluted with DCM (100 mL), filtered, and the filter cake washed with further DCM (3×50 mL). The mother liquor was then concentrated under reduced pressure, and the residue purified by silica gel chromatography. Fractions containing the product were pooled and lyophilized to give title compound F9. MS (m/z): 601.4 [M+H]⁺. ¹H NMR (400 MHz, CDCl₃) δ 7.96-7.90 (m, 1H), 7.50 (d, J=1.9 Hz, 1H), 3.48 (t, J=7.2 Hz, 2H), 3.38 (dq, J=18.7, 6.8 Hz, 1H), 2.59 (s, 3H), 2.14 (t, J=7.1 Hz, 2H), 1.57 (s, 16H), 1.51 (s, 18H), 1.46 (s, 8H), 1.21 (d, J=6.7 Hz, 5H), 0.83 (s, 10H), −0.05 (s, 7H) ppm.

Synthesis of tert-butyl 3-((di-tert-butoxyphosphoryl)oxy)-4-(4-hydroxy-2-methylbutan-2-yl)-5-methylbenzoate (F10): To a solution of F10 (14.7 mmol) in 50 mL of THE was added TBAF solution (1.0M in THF, 20 mmol, 1.5 equiv). When full conversion was observed, the reaction was concentrated under reduced pressure and transferred to a separatory funnel using DCM (200 mL) and water (200 mL). The organic layer was washed with brine (4×200 mL), dried over Na₂SO₄, concentrated under reduced pressure and the residue purified by silica gel chromatography. Fractions containing the product were pooled and concentrated under reduced pressure to give title compound F10. MS (m/z): 486.21 [M+H]⁺.

Synthesis of 3-(4-(tert-butoxycarbonyl)-2-((di-tert-butoxyphosphoryl)oxy)-6-methylphenyl)-3-methylbutanoic acid (Intermediate F): To a solution of F10 (9.68 mmol), TEMPO (0.484 mmol, 0.05 equiv), KHPO₄ (4.84 mmol, 0.5 equiv), and K₂HPO₄ (4.84 mmol, 0.5 equiv) in 100 mL of H₂O/MeCN (1:1) at 0° C. were added sequentially sodium hypochlorite (8.25% aqueous solution, 11.8 mmol, 1.22 equiv) and sodium chlorite (14.5 mmol, 1.5 equiv). The solution was stirred for 1 h at 0° C. and then warmed to rt. When full conversion was observed, the reaction was quenched with saturated sodium sulfite solution and the contents were transferred to a separatory funnel using DCM (150 mL) and H₂O (150 mL) where the pH was carefully adjusted to 2 with 1M HCl solution. The aqueous layer was extracted with DCM (2×100 mL) and the organic fractions were collected, dried over Na₂SO₄, concentrated under reduced pressure. The title compound was obtained by recrystallization from EtOAc/Hexanes to yield title compound Intermediate F. MS (m/z): 523.29 [M+Na]⁺.

### 1.6 Synthesis of Intermediate G

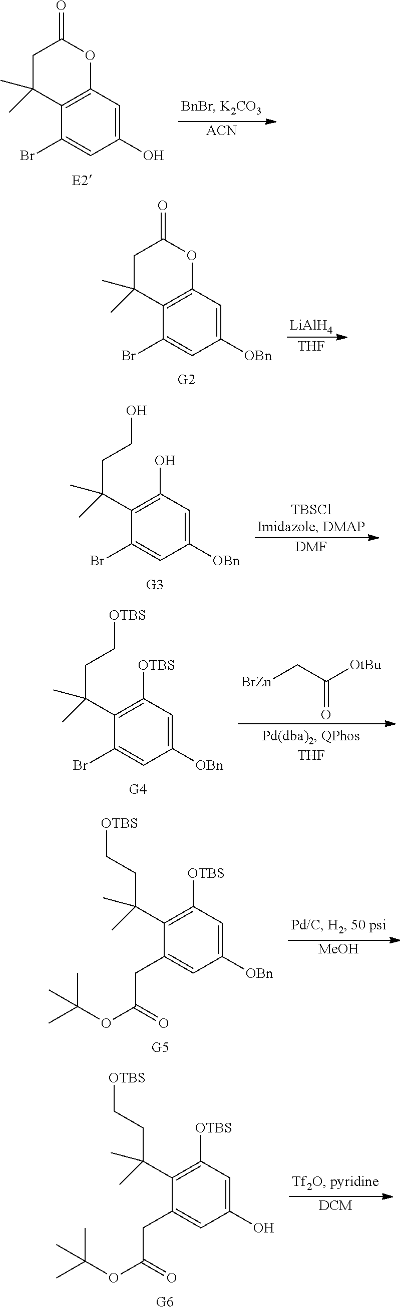

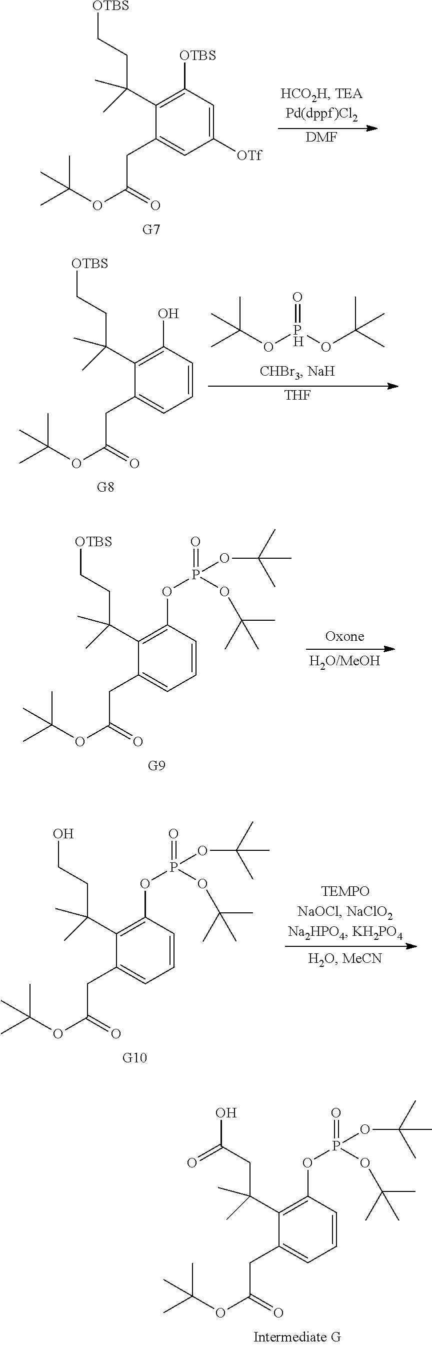

Synthesis of 7-(benzyloxy)-5-bromo-4,4-dimethylchroman-2-one (G2): A flask was charged with E2′ (1.0 eq) and ACN (0.7 M). Potassium carbonate (2.0 eq) was added, followed by dropwise addition of benzyl bromide (1.5 eq). The reaction was heated at 80° C. for 12 hours. The reaction was concentrated, diluted with water and extracted with DCM (3×). The combined organic layers were washed with brine, dried over Na₂SO₄, filtered and concentrated under reduced pressure. The residue was purified by silica gel chromatography to yield title compound G2. MS (m/z): 361.2 [M+H]⁺.

Synthesis of 5-(benzyloxy)-3-bromo-2-(4-hydroxy-2-methylbutan-2-yl)phenol (G3): A flask was charged with LiAlH₄ (1.30 eq) and THE (0.5 M). The mixture was cooled to 0° C., G2 (1.0 eq) was added and the reaction was stirred at RT for 3 hours. The reaction was cooled to 0° C., water was added, followed by 1 M HCl. The reaction was extracted with EtOAc (3×), the combined organic layers were washed with sat. NaHCO₃(aq) (2×) until pH=7-8. The organic layer was washed with brine, dried over Na₂SO₄, filtered and concentrated under reduced pressure to yield title compound G3 which was used without further purification. MS (m/z): 363.2/365.2 [M−H]⁻.

Synthesis of (3-(4-(benzyloxy)-2-bromo-6-((tert-butyldimethylsilyl)oxy)phenyl)-3-methylbutoxy)(tert-butyl)dimethylsilane (G4): A flask was charged with G3 (1.0 eq) and DMF (0.5 M). Imidazole (5.0 eq), TBSCl (5.2 eq) and DMAP (0.1 eq) were added and the mixture was heated at 65° C. for 12 hours. The reaction was poured into an ice-water mixture and was extracted with EtOAc (3×). The combined organic layers were washed with brine (3×), dried over Na₂SO₄ and concentrated under reduced pressure. The residue was purified by silica gel chromatography to yield title compound G4. ¹H NMR (400 MHz, CDCl₃) δ 7.47-7.31 (m, 5H), 6.91 (d, J=2.8 Hz, 1H), 6.35 (d, J=2.8 Hz, 1H), 4.98 (s, 2H), 3.56-3.43 (m, 2H), 2.32-2.18 (m, 2H), 1.60 (s, 6H), 0.98 (s, 9H), 0.85 (s, 9H), 0.26 (s, 6H), −0.02 (s, 6H).

Synthesis of tert-butyl 2-(5-(benzyloxy)-3-((tert-butyldimethylsilyl)oxy)-2-(4-((tert-butyldimethylsilyl)oxy)-2-methylbutan-2-yl)phenyl)acetate (G5): A flask was charged with G4 (1.0 eq) in THE (0.3 M). 2-Tert-butoxy-2-oxoethylzinc bromide (3.0 eq), Pd(dba)₂ (0.1 eq) and XPhos (0.1 eq) were added to the mixture and the reaction was heated at 80° C. for 12 hours. The reaction was cooled to RT, quenched with a 0.5 M aqueous solution of citric acid and extracted with n-heptanes. The organic layer was washed with sat. NaHCO₃(aq) until pH=7-8. The organic layer was washed with brine, dried over Na₂SO₄, filtered and concentrated under reduced pressure. The residue was purified by silica gel chromatography to yield title compound G5. ¹H NMR (400 MHz, DMSO-d₆) δ 7.42-7.29 (m, 5H), 6.39 (d, J=2.8 Hz, 1H), 6.25 (d, J=2.8 Hz, 1H), 5.02 (s, 2H), 3.76 (s, 2H), 3.40-3.33 (m, 2H), 2.07 (t, J=7.8 Hz, 2H), 1.44-1.35 (m, 15H), 0.95 (s, 9H), 0.79 (s, 9H), 0.22 (s, 6H), −0.09 (s, 6H).

Synthesis of tert-butyl 2-(3-((tert-butyldimethylsilyl)oxy)-2-(4-((tert-butyldimethylsilyl)oxy)-2-methylbutan-2-yl)-5-hydroxyphenyl)acetate (G6): A bottle was charged with G5 (1.0 eq), MeOH (0.2 M) and Pd/C (0.1 eq). The reaction was degassed with vacuum/hydrogen (3×). The reaction was heated under hydrogen (50 psi) at 50° C. for 2 hours. The mixture was filtered over a pad of celite and the cake was washed with MeOH (3×). The filtrate was concentrated under reduce pressure and the residue was purified by silica gel chromatography to yield title compound G6.

Synthesis of tert-butyl 2-(3-((tert-butyldimethylsilyl)oxy)-2-(4-((tert-butyldimethylsilyl)oxy)-2-methylbutan-2-yl)-5-(((trifluoromethyl)sulfonyl)oxy)phenyl)acetate (G7): A flask charged with G6 (1.00 eq) and DCM (0.5 M) was cooled to 0° C. Pyridine (2.00 eq) was added, followed by Tf₂O (1.25 eq), and the reaction was stirred at RT for 12 hours. The reaction was concentrated under reduced pressure, poured into water, extracted with DCM (3×), dried over Na₂SO₄, filtered and concentrated under reduced pressure. The residue was purified by silica gel chromatography to yield title compound G7. ¹H NMR (400 MHz, CDCl₃) δ 6.68 (d, J=2.8 Hz, 1H), 6.62 (d, J=2.8 Hz, 1H), 3.82 (s, 2H), 3.43 (t, J=7.4 Hz, 2H), 2.15 (t, 2H), 1.50 (s, 6H), 1.45 (s, 9H), 1.03 (s, 9H), 0.83 (s, 9H), 0.33 (s, 6H), −0.06 (s, 6H).

Synthesis of tert-butyl 2-(2-(4-((tert-butyldimethylsilyl)oxy)-2-methylbutan-2-yl)-3-hydroxyphenyl)acetate (G8): A vial was charged with G7 (1.34 mmol), Pd(dppf)Cl₂ (0.02 eq), DMF (6.0 mL), triethylamine (4.02 mmol) and formic acid (2.68 mmol) under nitrogen. The mixture was heated at 60° C. for 3 hours then RT for 18 hours. The reaction was quenched with sat. NH₄Cl_{(aq)} and extracted with EtOAc (2×). The combined organic layers were washed with a 5% aqueous solution of LiCl (2×), dried over MgSO₄, filtered and concentrated. The residue was purified by silica gel chromatography to yield title compound G8. MS (m/z): 407.3 [M−H]⁻.

Synthesis of tert-butyl 2-(2-(4-((tert-butyldimethylsilyl)oxy)-2-methylbutan-2-yl)-3-((di-tert-butoxyphosphoryl)oxy)phenyl)acetate (G9): To a solution of G8 (1.34 mmol) in THE (5.0 mL) at 0° C. was added di-tert-butyl phosphite (1.34 mmol) and bromoform (1.81 mmol). The solution was stirred for 5 minutes. Sodium hydride (60% dispersion in mineral oil, 1.81 mmol) was then added in one portion. Upon reaction completion, the reaction was quenched with water and extracted with EtOAc (3×). The combined organic layers were washed with brine, dried over Na₂SO₄, filtered and concentrated. The residue was purified by silica gel chromatography (eluent: hexanes/EtOAc) to yield title compound G9. MS (m/z): 623.0 [M+Na]⁺.

Synthesis of tert-butyl 2-(3-((di-tert-butoxyphosphoryl)oxy)-2-(4-hydroxy-2-methylbutan-2-yl)phenyl)acetate (G10): To a mixture of G9 (4.89 mmol) in H₂O/MeOH (1:1, 15 mL) at 0° C. was added Oxone® (7.34 mmol). The ice bath was removed and the reaction was stirred at RT for 2 hours. The reaction was quenched with saturated aqueous sodium sulfite and extracted with EtOAc (3×). The combined organic layers were washed with brine, dried over Na₂SO₄, filtered and concentrated under reduced pressure. The residue was purified by silica gel chromatography (eluent: hexanes/EtOAc) to yield title compound G10. MS (m/z): 509.0 [M+Na]⁺.

Synthesis of 3-(2-(2-(tert-butoxy)-2-oxoethyl)-6-((di-tert-butoxyphosphoryl)oxy)phenyl)-3-methylbutanoic acid (Intermediate G): To a solution of G10 (2.80 mmol) in ACN/water (1:1, 28 mL) was added TEMPO (0.21 mmol), potassium dihydrogen phosphate (1.40 mmol) and disodium hydrogen phosphate (1.40 mmol). The solution was cooled to 0° C. Sodium chlorite (4.19 mmol) was added, followed by sodium hypochlorite (8.25% NaOCl (aq), 3.41 mmol). The ice bath was removed and the reaction was stirred for 3.5 hours. The reaction was quenched with saturated aqueous sodium sulfite. The pH was adjusted to 2 with 1M aqueous HCl and the mixture was extracted with EtOAc (3×). The combined organic layers were washed with brine, dried over Na₂SO₄, filtered and concentrated under reduced pressure to yield Intermediate G which was used without further purification. MS (m/z): 523.0 [M+Na]⁺.

### 1.7 Synthesis of Intermediate H

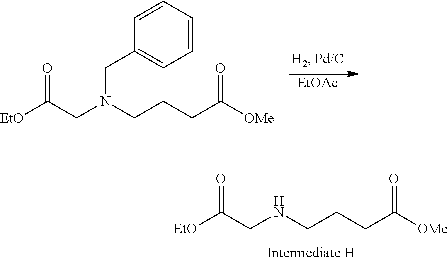 [SMILES: "EtOAc" ; CCOC(=O)CN(CCCC(=O)OC)Cc1ccccc1 ; CCOC(=O)CNCCCC(=O)OC]

To a solution of methyl 4-(benzyl(2-ethoxy-2-oxoethyl)amino)butanoate (1.79 mmol) in EtOAc (10 mL) was added palladium on carbon (0.358 mmol). The reaction was placed under an atmosphere of hydrogen and stirred at rt. Upon completion of the reaction, the mixture was filtered through Celite® and the filter cake was rinsed with EtOAc. The filtrate was concentrated and used directly as Intermediate H.

### 1.8 Synthesis of Intermediate I

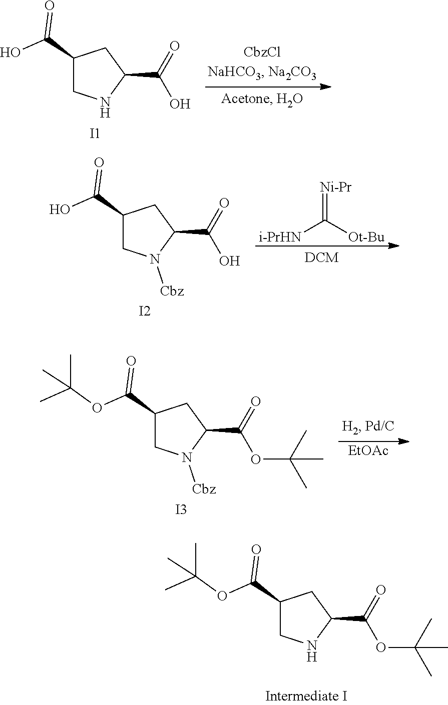

Synthesis of (2S,4S)-1-((benzyloxy)carbonyl)pyrrolidine-2,4-dicarboxylic acid (I2): To a solution of (2S,4S)-pyrrolidine-2,4-dicarboxylic acid I1 (3.14 mmol) in water (5.0 mL) was added sodium carbonate (9.43 mmol) and sodium bicarbonate (3.14 mmol). Acetone (0.5 mL) was added followed by benzyl chloroformate (6.91 mmol). To push the reaction to completion, additional benzyl chloroformate (6.91 mmol) was added to the reaction mixture. Upon completion of the reaction, DCM (5 mL) was added to the reaction, and the mixture was acidified to pH 2 with aqueous 1N HCl. The organics were separated, and aqueous layer extracted with Me-THF (5 mL×3). The combined organic layers were dried over sodium sulfate, filtered, and concentrated. The crude material was purified by reverse-phase MPLC to afford (2S,4S)-1-((benzyloxy)carbonyl)pyrrolidine-2,4-dicarboxylic acid I2. MS (m/z): 292.3 [M−H]⁻.

Synthesis of 1-benzyl 2,4-di-tert-butyl (2S,4S)-pyrrolidine-1,2,4-tricarboxylate (I3): To a solution of (2S,4S)-1-((benzyloxy)carbonyl)pyrrolidine-2,4-dicarboxylic acid H2 (0.682 mmol) in DCM (6.8 mL) was added 2-tert-butyl-1,3-diisopropylurea (6.82 mmol). Upon completion of the reaction, the mixture was filtered through Celite® and the filter cake was rinsed with DCM. The filtrate was concentrated and purified by silica gel chromatography to afford 1-benzyl 2,4-di-tert-butyl (2S,4S)-pyrrolidine-1,2,4-tricarboxylate I3. MS (m/z): 427.9 [M+Na]⁺.

Synthesis of di-tert-butyl (2S,4S)-pyrrolidine-2,4-dicarboxylate (Intermediate I): To a solution of 1-benzyl 2,4-di-tert-butyl (2S,4S)-pyrrolidine-1,2,4-tricarboxylate I3 (0.417 mmol) in EtOAc (4 mL) was added palladium on carbon (0.0834 mmol). The reaction was placed under an atmosphere of hydrogen and stirred at rt. Upon completion of the reaction, the mixture was filtered through Celite® and the filter cake was rinsed with EtOAc. The filtrate was concentrated and used directly as Intermediate I.

### 1.9 Synthesis of Intermediate J

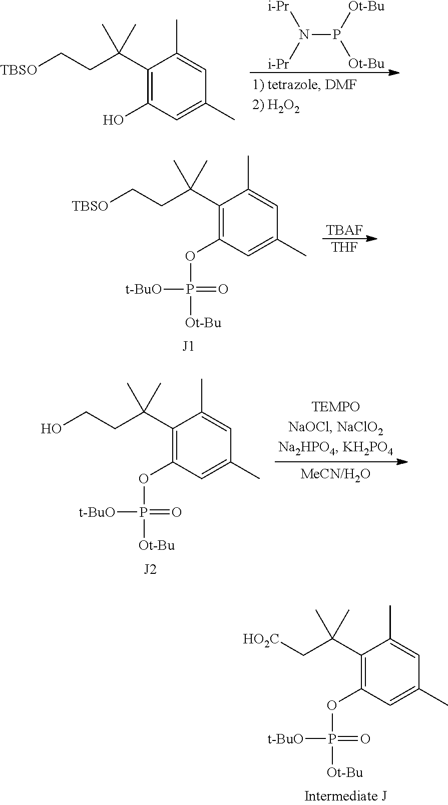

Synthesis of di-tert-butyl (2-(4-((tert-butyldimethylsilyl)oxy)-2-methylbutan-2-yl)-3,5-dimethylphenyl) phosphate (J1): To a stirred solution of 1-H-tetrazole (372 mmol) in MeCN (843 mL) and THE (123 mL) was added 2-(4-((tert-Butyldimethylsilyl)oxy)-2-methylbutan-2-yl)-3,5-dimethylphenol (62.0 mmol) and the mixture was cooled to 0° C. Di-t-butyl-N,N-diisopropylphosphoramidite (372 mmol) was then added and the reaction mixture was stirred at room temperature while monitored by TLC and LCMS. After 12 h, the reaction mixture was cooled to 0° C. and treated with aq. H₂O₂ solution (60 mL, 30 wt. %). The reaction was gradually warmed to room temperature and stirred. After completion, the reaction mixture was diluted with EtOAc and washed with sat. aq. sodium thiosulfate solution. The combined organic layers were dried over anhydrous Na₂SO₄, filtered, and concentrated. The crude mixture was purified by column chromatography to afford title compound J1. MS (m/z) 537.5 [M+Na]⁺.

J1 can also be prepared according to the following method: To a solution of 2-(4-((tert-Butyldimethylsilyl)oxy)-2-methylbutan-2-yl)-3,5-dimethylphenol (0.155 mmol) in 0.3 mL of THE at 0° C. were added sequentially di-tert-butyl phosphite (0.207 mmol) and bromoform (0.211 mmol) and finally Cs₂CO₃ (0.225 mmol). The resulting suspension was gradually warmed to rt, with continued stirring. After completion, the reaction mixture was quenched with water and extracted with ethyl acetate. The organic fractions were combined, dried over Na₂SO₄, concentrated under reduced pressure, and purified by column chromatography (eluting with 0 to 60% EtOAc in hexanes) to afford title compound J1. MS (m/z) 537.5 [M+Na]⁺.

Synthesis of di-tert-butyl (2-(4-hydroxy-2-methylbutan-2-yl)-3,5-dimethylphenyl) phosphate (J2): To a stirred solution of J1 (9.7 mmol) in THE (50 mL) at 0° C. was added TBAF (20 mL, 1 M in THF) and the reaction mixture was stirred at room temperature for 12 h. After completion, the reaction mixture was diluted with EtOAc, washed with sat. aq. solutions of NH₄Cl and brine. The combined organic layers were dried over anhydrous Na₂SO₄, filtered, and concentrated to yield title compound J2, which was directly used in the subsequent reaction without purification.

Synthesis of 3-(2-((di-tert-butoxyphosphoryl)oxy)-4,6-dimethylphenyl)-3-methylbutanoic acid (Intermediate J): To a stirred solution of J2 (7.11 mmol) in MeCN (32 mL) and water (32 mL) was added TEMPO (0.36 mmol), KH₂PO₄ (3.5 mmol), and Na₂HPO₄ (3.5 mmol) then the mixture was cooled to 0° C. NaClO (8.6 mmol, 10 wt. %) and NaClO₂ (10.6 mmol) were then added and the reaction was gradually warmed to room temperature and stirred. After completion, the reaction mixture was diluted with EtOAc and washed with sat. aq. sodium thiosulfate solution. The combined organic layers were dried over anhydrous Na₂SO₄, filtered, and concentrated. The crude mixture was purified by column chromatography to afford title compound Intermediate J. MS (m/z) 415.1 [M+H]⁺.

### 1.10 Synthesis of Intermediate K

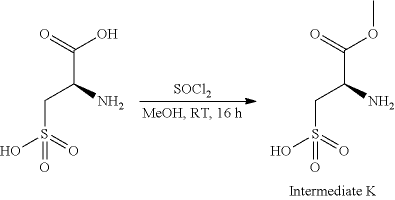

To a suspension of L-cysteic acid (29.6 mmol) in methanol (120 mL) at 0° C. was added thionyl chloride (296 mmol) dropwise at a rate that maintained the internal temperature below 10° C. The reaction was allowed to warm to RT and stirred for 16 hours. The reaction mixture was concentrated in vacuo and the residue suspended in diethyl ether (100 mL) and sonicated for 1 h. The resulting solid was isolated by vacuum filtration to provide (R)-2-amino-3-methoxy-3-oxopropane-1-sulfonic acid (Intermediate K).MS (m/z): 184.2 [M+H]⁺. ¹H NMR (400 MHz, Deuterium Oxide) δ 4.61 (dd, J=7.0, 4.1 Hz, 1H), 3.89 (s, 3H), 3.61-3.46 (m, 2H).

### 1.11 Synthesis of Intermediate L

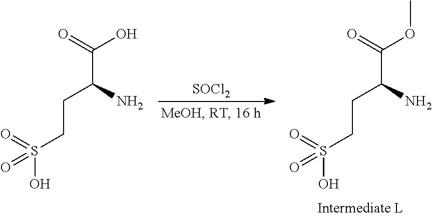

Intermediate L was synthesized using the method described for Intermediate K, substituting L-homocysteic acid for L-cysteic acid. MS (m/z): 198.2 [M+H]⁺. ¹H NMR (400 MHz, Deuterium Oxide) 6 4.36 (t, J=6.6 Hz, 1H), 3.88 (s, 3H), 3.21-3.00 (m, 2H), 2.41 (dddt, J=42.0, 15.2, 9.0, 6.5 Hz, 2H).

### 1.12 Synthesis of Intermediate M

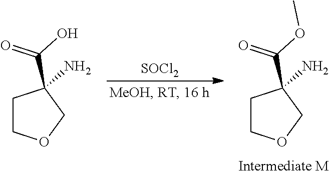

Methyl (S)-3-aminotetrahydrofuran-3-carboxylate (Intermediate M) was synthesized using the method described for Intermediate K, substituting (S)-3-aminotetrahydrofuran-3-carboxylic acid for L-cysteic acid. MS (m/z): 146.1 [M+H]⁺.

### 1.13 Synthesis of Intermediate N

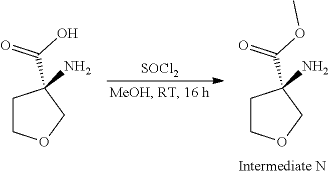

Methyl (R)-3-aminotetrahydrofuran-3-carboxylate (Intermediate N) was synthesized using the method described for Intermediate K, substituting (R)-3-aminotetrahydrofuran-3-carboxylic acid for L-cysteic acid. MS (m/z): 146.1 [M+H]⁺.

### 1.14 Synthesis of Intermediate O

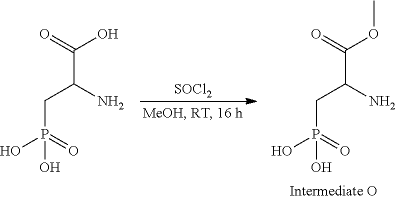

(2-amino-3-methoxy-3-oxopropyl)phosphonic acid (Intermediate O) was synthesized using the method described for Intermediate K, substituting 2-amino-3-phosphonopropanoic acid for L-cysteic acid. MS (m/z): 184.2 [M+H]⁺.

### 1.15 Synthesis of Intermediate P

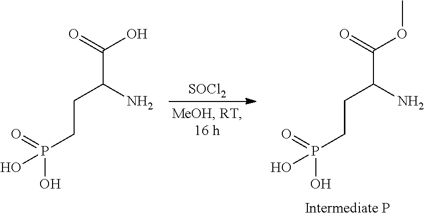

(3-amino-4-methoxy-4-oxobutyl)phosphonic acid (Intermediate P) was synthesized using the method described for Intermediate K, substituting 2-amino-4-phosphonobutanoic acid for L-cysteic acid. MS (m/z): 198.2 [M+H]⁺.

### 1.16 Synthesis of Intermediate Q

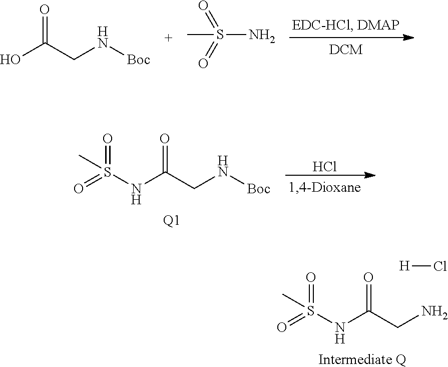

Synthesis of tert-butyl (2-(methylsulfonamido)-2-oxoethyl)carbamate (Q1): To a solution of Boc-L-glycine (5.71 mmol) and DMAP (22.8 mmol) in DCM (30 mL) was added methansulfonamide (6.85 mmol) followed by EDC-HCl (6.85 mmol). The RM was stirred at RT for 3 h, then diluted with DCM (30 mL) and washed with aqueous HCl solution (1N, 15 mL) twice. The isolated organic layer was dried over sodium sulfate, isolate by vacuum filtration, and concentrated in vacuo to provide crude tert-butyl (2-(methylsulfonamido)-2-oxoethyl)carbamate (Q1) which was used without purification. MS (m/z): 275.2 [M+Na].

Synthesis of 2-amino-N-(methylsulfonyl)acetamide hydrochloride (Q): To a solution of crude Q1 (1.98 mmol) in 1,4-dioxane (5.0 mL) was added hydrochloride acid (4M in 1,4-dioxane, 4.94 mL). The RM was stirred at RT for 16 h. The RM was concentrated in vacuo to provide crude 2-amino-N-(methylsulfonyl)acetamide hydrochloride (Intermediate Q) which was used without purification. MS (m/z): 153.2 [M+H]⁺.

### 1.17 Synthesis of Intermediate R

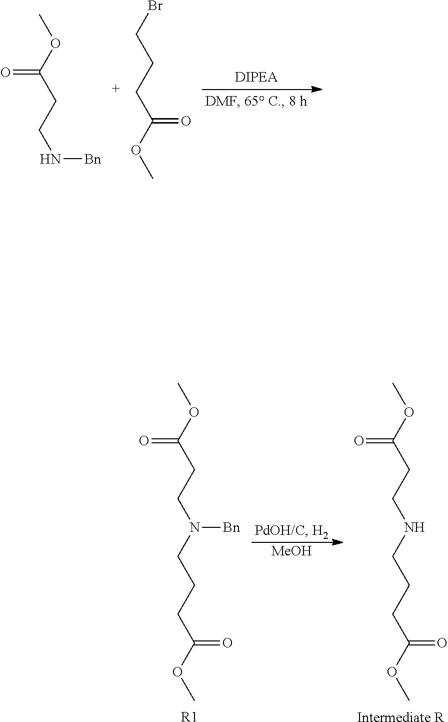

Synthesis of methyl 4-(benzyl(3-methoxy-3-oxopropyl)amino)butanoate (R1): To a solution of methyl 3-(benzylamino)propanoate (2.59 mmol) and methyl 4-bromobutanoate (3.31 mmol) in DMF (2.5 mL) was added DIPEA. The RM was stirred at 65° C. for 8 hours. The RM was concentrated in vacuo and purified by silica gel column chromatography (eluent: DCM/MeOH) to provide methyl 4-(benzyl(3-methoxy-3-oxopropyl)amino)butanoate (R1). MS (m/z): 294.2 [M+H]⁺. ¹H NMR (400 MHz, Chloroform-d) δ 7.29 (d, J=6.9 Hz, 4H), 7.23 (td, J=6.0, 2.3 Hz, 1H), 3.65 (s, 3H), 3.62 (s, 3H), 3.55 (s, 2H), 2.79 (t, J=7.1 Hz, 2H), 2.45 (dt, J=11.1, 7.0 Hz, 4H), 2.30 (t, J=7.4 Hz, 2H), 1.77 (p, J=7.1 Hz, 2H).

Synthesis of methyl 4-((3-methoxy-3-oxopropyl)amino)butanoate (Intermediate R): To a solution of R¹ (2.07 mmol) in MeOH (10.0 mL) was added 20% palladium hydroxide on carbon (0.21 mmol). The flask was vacuum purged and backflushed with hydrogen gas three time and then stirred under a hydrogen atmosphere for 30 minutes. The reaction mixture was filtered through a pad of Celite, then concentrated in vacuo to provide crude methyl 4-((3-methoxy-3-oxopropyl)amino)butanoate (Intermediate R) which was used without purification. MS (m/z): 204.2 [M+H]⁺.

### 1.18 Synthesis of Intermediate S

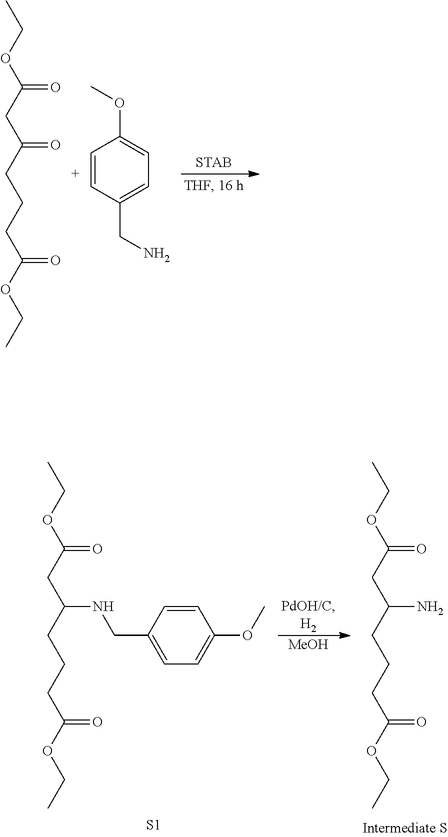

Synthesis of diethyl 3-((4-methoxybenzyl)amino)heptanedioate (S1): To a solution of diethyl 3-oxoheptanedioate (2.17 mmol) and (4-methoxyphenyl)methanamine (8.69 mmol) in THE (20.0 mL) was added sodium triacetoxyborohydride (8.69 mmol). The RM was stirred at RT for 16 h. The RM was concentrated in vacuo and purified by silica gel column chromatography (eluent: DCM/MeOH) to provide diethyl 3-((4-methoxybenzyl)amino)heptanedioate (S1). MS (m/z): 352.2 [M+H]⁺.

Synthesis of diethyl 3-aminoheptanedioate (Intermediate S): Intermediate S was synthesized using the method described in Step 2 of Example 1.17, substituting S1 for R1. MS (m/z): 232.2 [M+H]⁺.

### 1.19 Synthesis of Intermediate T

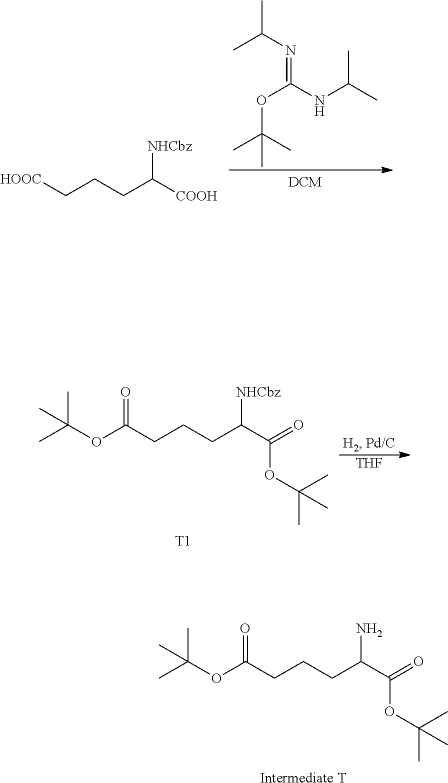

Synthesis of di-tert-butyl 2-(((benzyloxy)carbonyl)amino)hexanedioate (T1): N,N′-diisopropyl-O-tert-butyl isourea (0.881 mmol) was added to a suspension of 2-(benzyloxycarbonylamino)hexanedioic acid (0.339 mmol) in DCM (3.4 mL) at room temperature and stirred at the same temperature for 15 hr. Additional N,N′-diisopropyl-O-tert-butyl isourea (0.881 mmol) was then added to the reaction mixture and the vessel was sealed and heated to 50° C. for 60 hr. The solids were then filtered off and washed with DCM. The filtrate was concentrated and the product was isolated by silica gel column chromatography (eluent: EtOAc/hexanes) to give di-tert-butyl 2-(((benzyloxy)carbonyl)amino)hexanedioate T1. ¹H NMR (400 MHz, DMSO-d₆) δ 7.62 (m, 1H), 7.35 (m, 5H), 5.03 (m, 2H), 3.87 (m, 1H), 2.17 (m, 2H), 1.59 (m, 4H), 1.39 (m, 18H)

Synthesis of di-tert-butyl 2-aminohexanedioate (Intermediate T): 10% Pd/C (10.0 mg) was added to a solution of di-tert-butyl 2-(((benzyloxy)carbonyl)amino)hexanedioate Intermediate T1 (0.137 mmol) in THE (10.0 mL) and the reaction mixture was placed under an atmosphere of H₂ (1 atm) and stirred vigorously at room temperature for 6 hr. The reaction mixture was filtered over a pad of celite and concentrated. The product was isolated by silica gel column chromatography (eluent: EtOAc/hexanes) to give di-tert-butyl 2-aminohexanedioate (Intermediate T). ¹H NMR (400 MHz, CDCl₃) δ 3.31 (m, 1H), 2.24 (m, 2H), 1.65 (m), 1.47 (m, 18H))

### 1.20 Synthesis of Intermediate U

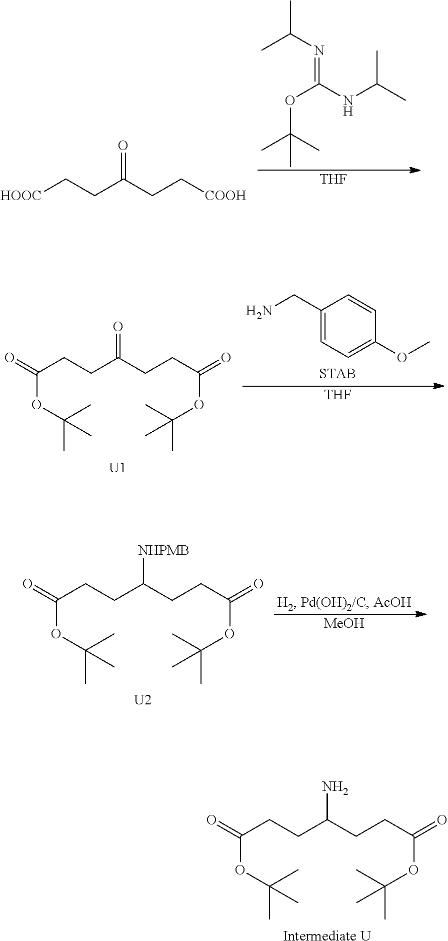

Synthesis of di-tert-butyl 4-oxoheptanedioate (U1): N,N′-diisopropyl-O-tert-butyl isourea (8.61 mmol) was added to a suspension of 2-(benzyloxycarbonylamino)hexanedioic acid (1.72 mmol) in THE (6.0 mL) at room temperature and stirred at the same temperature for 4 hr. Additional N,N′-diisopropyl-O-tert-butyl isourea (4.30 mmol) was then added to the reaction mixture and stirred for a further 16 hr at room temperature. The solids were then filtered off and washed with hexanes. The filtrate was concentrated, and the product was isolated by silica gel column chromatography (eluent: EtOAc/hexanes) to give di-tert-butyl 4-oxoheptanedioate U1. ¹H NMR (400 MHz, Chloroform-d) δ 2.73 (t, J=6.6 Hz, 4H), 2.54 (t, J=6.6 Hz, 4H), 1.45 (s, 18H).

Synthesis of di-tert-butyl 4-((4-methoxybenzyl)amino)heptanedioate (U2): Sodium triacetoxyborohydride (1.91 mmol) was added to a solution of (4-methoxyphenyl)methanamine (1.91 mmol) of di-tert-butyl 4-oxoheptanedioate U1 (0.478 mmol) and in THE (4.8 mL) and the reaction mixture was stirred vigorously at room temperature for 48 hr. The reaction mixture was quenched with saturated aqueous NaHCO₃ and the aqueous layer was extracted 3 times with EtOAc. The combined organic layers were dried over sodium sulfate and concentrated. The product was isolated by RP-MPLC (eluent: water/MeCN 0.1% TFA) to give di-tert-butyl 4-((4-methoxybenzyl)amino)heptanedioate U2. ¹H NMR (400 MHz, Chloroform-d) δ 7.44-7.37 (m, 2H), 6.95-6.88 (m, 2H), 4.23 (s, 2H), 3.83 (s, 3H), 3.21-2.77 (m), 2.51 (m, 2H), 2.27 (m, 2H), 2.14 (m, 2H), 1.95-1.83 (m, 2H), 1.45 (s, 18H).

Synthesis of di-tert-butyl 4-aminoheptanedioate (Intermediate U): Acetic acid (0.493 mmol) was added to a solution of di-tert-butyl 4-((4-methoxybenzyl)amino)heptanedioate U2 (0.135 mmol) in methanol (5.0 mL) followed by 20% Pd(OH)₂/C (8.46 mg). The mixture was placed under an atmosphere of H₂ (1 atm) and stirred vigorously for 36 hr. The reaction mixture was filtered over celite and concentrated to provide di-tert-butyl 4-aminoheptanedioate (Intermediate U), which was used without further purification. ¹H NMR (400 MHz, Chloroform-d) δ 3.37-3.21 (m, 1H), 2.55-2.34 (m, 4H), 2.03-1.84 (m, 4H), 1.43 (s, 18H).

### 1.21 Synthesis of Intermediate V

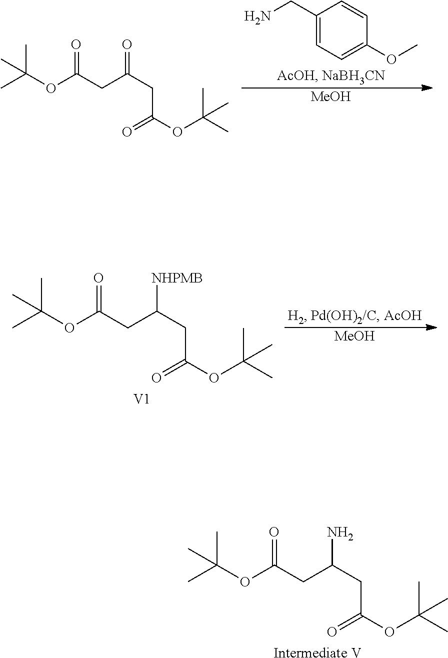

Synthesis of di-tert-butyl 3-((4-methoxybenzyl)amino)pentanedioate (V1): NaBH₃CN (1.39 mmol) was added to a solution of di-tert-butyl 3-oxopentanedioate (1.94 mmol) and (4-methoxyphenyl)methanamine (2.32 mmol) in methanol (3.5 mL) at room temperature and stirred for 1 hr. AcOH (2.32 mmol) was added to the reaction mixture and stirring was continued for 72 hr. Additional NaBH₃CN (1.39 mmol), (4-methoxyphenyl)methanamine (2.32 mmol), and AcOH (2.32 mmol) was added to mixture and stirred for a further 24 hr. The reaction mixture was then diluted with EtOAc (20 mL) and 1 M HCl (20 mL) was added. After stirring for 30 min, the pH of the aqueous layer was adjusted to pH=9 with saturated, aqueous NaHCO₃. The aqueous layer was extracted with EtOAc (×3) and the combined organic layers were washed with brine, dried over Na₂SO₄, and concentrated. The product was isolated by RP-HPLC (eluent: water/MeCN 0.1% TFA) to give di-tert-butyl 3-((4-methoxybenzyl)amino)pentanedioate V1. MS (m/z): 380.2 [M+H]⁺. ¹H NMR (400 MHz, Chloroform-d) δ 7.40-7.31 (m, 2H), 6.97-6.87 (m, 2H), 4.18 (m, 2H), 3.84-3.72 (m, 4H), 2.91 (ddd, J=17.6, 6.9, 2.7 Hz, 2H), 2.69 (ddd, J=17.6, 6.4, 2.0 Hz, 2H), 1.45 (s, 18H).

Synthesis of di-tert-butyl 3-aminopentanedioate (Intermediate V): AcOH (1.55 mmol) was added to a solution of V1 (1.27 mmol) in MeOH (20.0 mL) followed by 20% Pd(OH)₂/C (26.9 mg) and placed under an atmosphere of H₂ (1 atm). After stirring vigorously for 15 hr, the suspension was filtered over a pad of celite and the filtrate concentrated to give di-tert-butyl 3-aminopentanedioate (Intermediate V), which was used without further purification. ¹H NMR (400 MHz, Chloroform-d) δ 3.84 (m, 1H), 2.83 (m, 2H), 2.68 (m, 2H), 1.46 (s, 18H).

### 1.22 Synthesis of Intermediate W

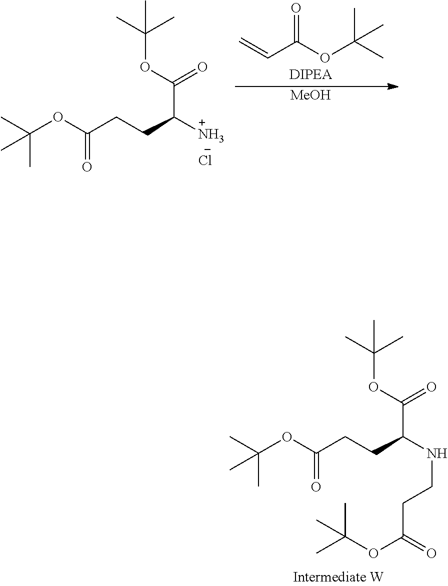

Synthesis of (Intermediate W): N,N-diisopropylethylamine (5.07 mmol) was added to a solution of di-tert-butyl L-glutamate hydrochloride (1.69 mmol) and tert-butyl prop-2-enoate (1.69 mmol) in methanol (3.5 mL) at room temperature and stirred for 96 hr. The reaction mixture was then diluted with EtOAc and saturated aqueous NaHCO₃. The aqueous layer (pH=9) was extracted with EtOAc (×3) and the combined organic layers were dried over Na₂SO₄, and concentrated. The product was isolated by silica gel column chromatography (eluent: EtOAc/hexanes) to give di-tert-butyl (3-(tert-butoxy)-3-oxopropyl)-L-glutamate (Intermediate W). MS (m/z): 388.158 [M+H]⁺. H NMR (400 MHz, Chloroform-d) δ 3.09 (dd, J=8.1, 5.6 Hz, 1H), 2.91 (dt, J=11.6, 6.8 Hz, 1H), 2.67 (dt, J=11.7, 6.6 Hz, 1H), 2.43-2.27 (m, 4H), 1.97-1.85 (m, 1H), 1.77 (m, 1H), 1.53-1.39 (m, 27H).

### 1.23 Synthesis of Intermediate X

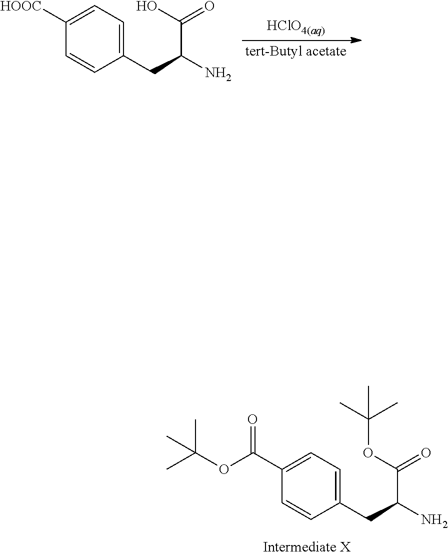 [SMILES: N[C@@H](Cc1ccc(C(=O)O)cc1)C(=O)O ; CC(C)(C)OC(=O)c1ccc(C[C@H](N)C(=O)OC(C)(C)C)cc1]

Synthesis of (Intermediate X): A 70% aqueous solution of perchloric acid (2.42 mmol) was added to a suspension of (S)-4-(2-amino-2-carboxyethyl)benzoic acid (0.956 mmol) in tert-butyl acetate (7.7 ml) at room temperature and stirred for 24 hr. The reaction mixture was then warmed to 35° C. and stirred for a further 12 hr. The reaction mixture was cooled to room temperature and poured into saturated aqueous NaHCO₃. The biphasic mixture was extracted with EtOAc (3×) and the combined organic layers were dried over Na₂SO₄ and concentrated. The product was isolated by silica gel column chromatography (eluent: EtOAc/hexanes) to give tert-butyl (S)-4-(2-amino-3-(tert-butoxy)-3-oxopropyl)benzoate (Intermediate X). ¹H NMR (400 MHz, Chloroform-d) δ 7.94-7.90 (m, 2H), 7.29-7.26 (m, 2H), 3.62 (dd, J=7.7, 5.6 Hz, 1H), 3.09 (dd, J=13.5, 5.6 Hz, 1H), 2.88 (dd, J=13.5, 7.7 Hz, 1H), 1.59 (m, 9H), 1.44 (s, 9H).

### 2. Example Compounds, Synthesis, and Characterization

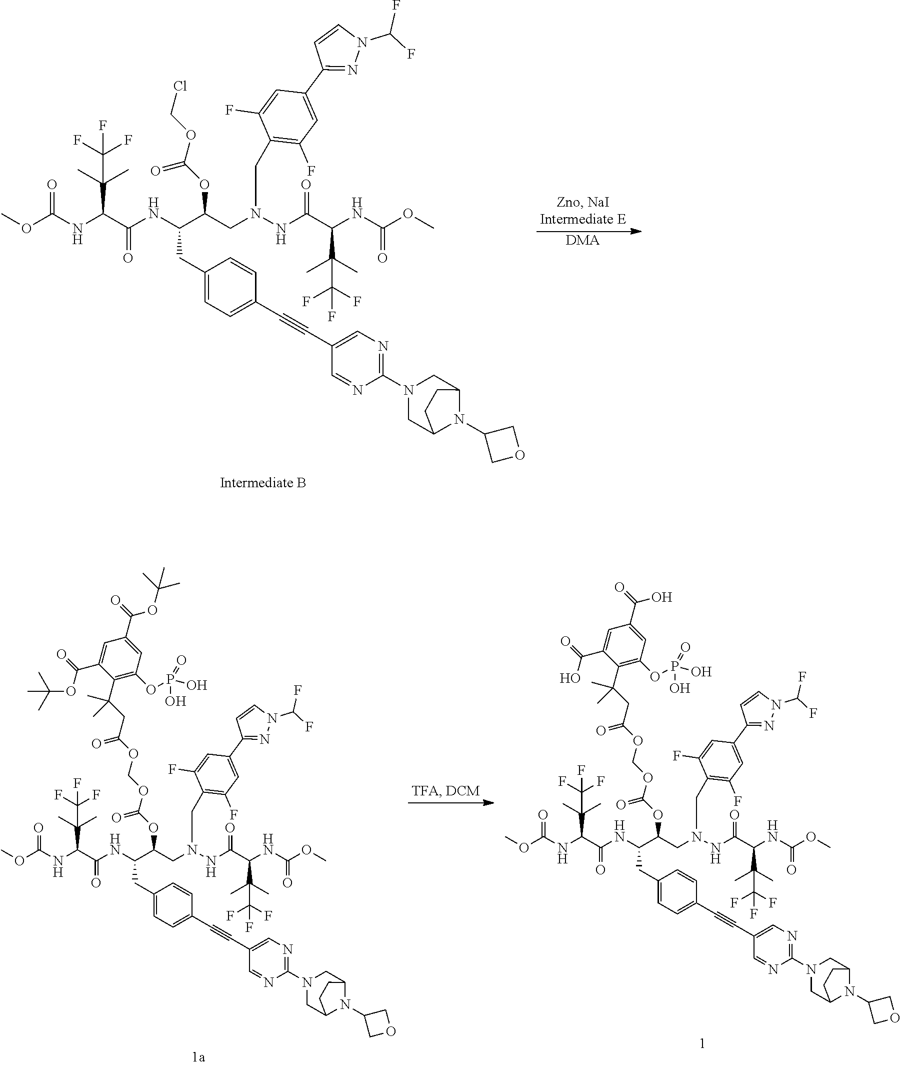

### Example 1

Synthesis of di-tert-butyl 4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-(phosphonooxy)isophthalate (1a): A flask charged with Intermediate B (0.080 mmol), 3-(2,4-bis(tert-butoxycarbonyl)-6-((di-tert-butoxyphosphoryl)oxy)phenyl)-3-methylbutanoic acid Intermediate E (0.112 mmol), zinc oxide (0.481 mmol), and sodium iodide (0.320 mmol) in DMA (1.0 mL) was heated to 65° C. for 3 hours. The reaction was cooled to rt and purified by reverse-phase HPLC (eluent: water/MeCN 0.1% TFA) to afford di-tert-butyl 4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-(phosphonooxy)isophthalate 1a. MS (m/z): 1686.1 [M+H]⁺.

Synthesis of 4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-(phosphonooxy)isophthalic acid (1): To a solution of 1a (0.00949 mmol) in DCM (0.4 mL) was added TFA (0.2 mL). The reaction mixture was stirred at rt for 90 minutes, then concentrated. The residue was purified by reverse-phase HPLC (eluent: water/MeCN 0.1% TFA) to provide 4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-(phosphonooxy)isophthalic acid 1. MS (m/z): 1574.0 [M+H]⁺. ¹H NMR (400 MHz, Methanol-d4) δ 8.56-8.45 (m), 8.21 (s, 1H), 8.14-8.05 (m), 7.73-7.27 (m), 7.21 (d, J=8.1 Hz, 1H), 7.15 (d, J=8.0 Hz, 1H), 7.05-6.79 (m), 5.80-5.56 (m), 5.04-4.70 (m), 4.67-4.42 (m), 4.39-4.20 (m), 4.17-3.95 (m), 3.74-3.63 (m), 3.51-3.43 (m), 3.36-3.34 (m), 3.27-3.03 (m), 2.93 (ddd, J=31.3, 13.1, 6.4 Hz, 1H), 2.85-2.59 (m), 2.29-2.11 (m), 2.02-1.91 (m), 1.72-1.62 (m), 1.29 (d, J=2.7 Hz, 1H), 1.23-1.07 (m), 1.04 (s, 3H). ¹⁹F NMR (376 MHz, Methanol-d4) δ −77.43 (s), −77.74 (d, J=16.5 Hz), −96.93 (d, J=6.4 Hz), −114.66 (s).

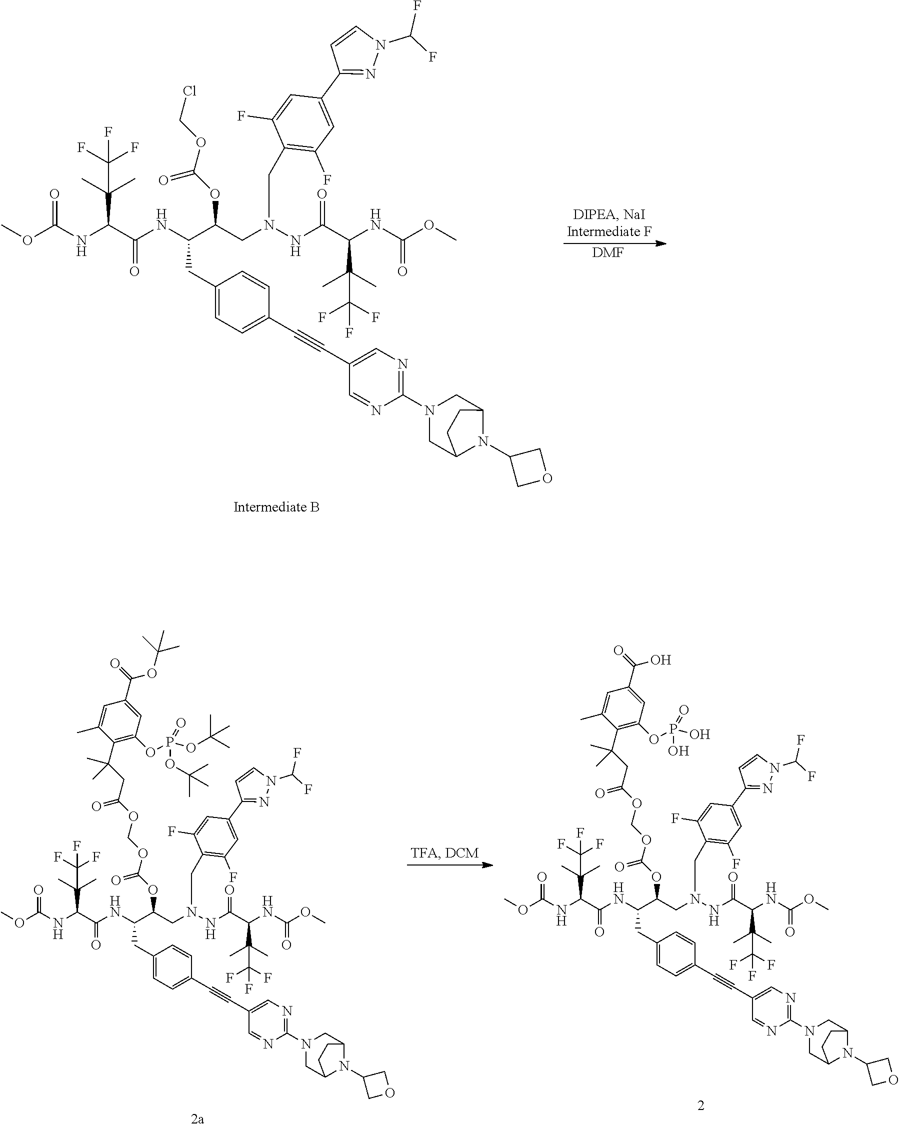

### Example 2

Synthesis of tert-butyl 3-((di-tert-butoxyphosphoryl)oxy)-4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methylbenzoate (2a): A flask charged with Intermediate B (0.22 mmol), 3-(4-(tert-butoxycarbonyl)-2-((di-tert-butoxyphosphoryl)oxy)-6-methylphenyl)-3-methylbutanoic acid Intermediate F (0.25 mmol), DIPEA (0.56 mmol), and sodium iodide (0.67 mmol) in DMF (1.7 mL) was heated to 65° C. for 6 hours. The reaction was cooled to rt and purified by reverse-phase HPLC (eluent: water/MeCN 0.1% TFA) to afford tert-butyl 3-((di-tert-butoxyphosphoryl)oxy)-4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methylbenzoate 2a. MS (m/z): 1712.2 [M+H]⁺.

Synthesis of 4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-3-methyl-5-(phosphonooxy)benzoic acid (2): To a solution of 2a (0.045 mmol) in DCM (0.45 mL) was added TFA (0.15 mL). The reaction mixture was stirred at rt for 2 h, then concentrated. The residue was purified by reverse-phase HPLC (eluent: water/MeCN 0.1% TFA) to provide 4-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-3-methyl-5-(phosphonooxy)benzoic acid 2. m/z): 1544.3 [M+H]⁺. ¹H NMR (400 MHz, Methanol-d4) δ 8.53 (s, 2H), 8.10 (d, J=2.7 Hz, 1H), 7.96 (s, 1H), 7.71-7.37 (m), 7.34 (d, J=7.8 Hz, 2H), 7.22 (d, J=8.0 Hz, 2H), 6.92 (d, J=2.8 Hz, 1H), 5.72 (d, J=5.8 Hz, 1H), 5.62 (d, J=5.9 Hz, 1H), 5.02-4.92 (m), 4.65-4.48 (m), 4.49-4.41 (m), 4.35-4.22 (m), 4.14 (s, 2H), 4.06 (d, J=13.2 Hz, 1H), 3.77-3.62 (m), 3.54-3.43 (m), 3.26-3.16 (m), 3.02-2.82 (m), 2.78-2.67 (m), 2.63 (s, 3H), 2.29-2.12 (m), 2.02-1.95 (m), 1.67 (s, 6H), 1.25-0.97 (m). ¹⁹F NMR (376 MHz, Methanol-d4) δ −77.43 (s), −77.70 (s), −96.85 (d, J=8.8 Hz), −97.01 (d, J=9.1 Hz), −114.65 (s). P NMR (162 MHz, Methanol-d4) δ −6.51 (d, J=6.3 Hz).

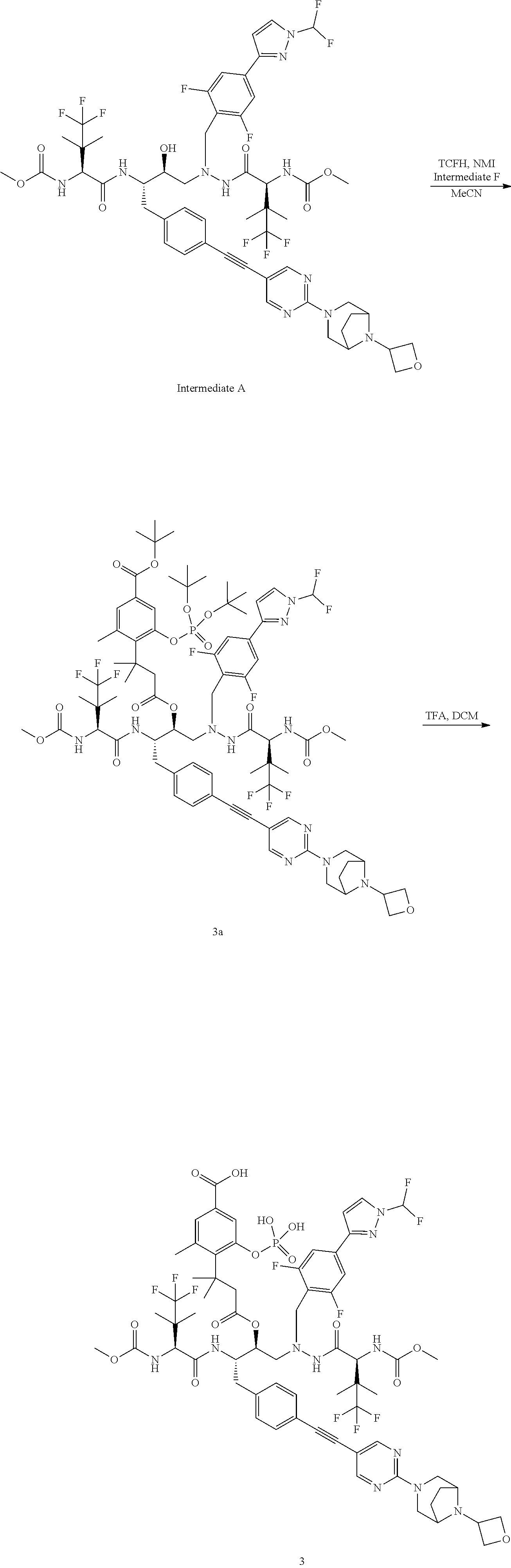

### Example 3

Synthesis of tert-butyl 3-((di-tert-butoxyphosphoryl)oxy)-4-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methylbenzoate (3a): To a solution of Intermediate A (0.22 mmol) in MeCN (2.0 mL) was added Intermediate F (0.22 mmol) and 1-methylimidazole (0.45 mmol), followed by TCFH (0.23 mmol). The reaction was stirred for 17 h, then heated to 35° C. for 1 h. The reaction was then cooled to rt and quenched with saturated aqueous ammonium chloride. The organics were separated, and aqueous layer extracted with DCM (3). The combined organic extracts were dried over Na₂SO₄, filtered, and concentrated. The crude material was purified by reverse-phase HPLC (eluent: water/MeCN 0.1% TFA) to provide the title compound 3a. MS (m/z): 1638.7 [M+H]⁻.

Synthesis of 4-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-methyl-5-(phosphonooxy)benzoic acid (3): To a solution of 3a (0.022 mmol) in DCM (0.22 mL) was added TFA (0.08 mL). The reaction mixture was stirred at rt for 2 h, then concentrated. The residue was purified by reverse-phase HPLC (eluent: water/MeCN 0.1% TFA) to provide 4-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-methyl-5-(phosphonooxy)benzoic acid 3. m/z): 1471.2 [M+H]⁺. ¹H NMR (400 MHz, Methanol-d4) δ 8.51 (s, 2H), 8.10 (d, J=2.7 Hz, 1H), 8.04 (s, 1H), 7.71-7.36 (m), 7.25 (d, J=7.8 Hz, 2H), 7.05 (d, J=7.9 Hz, 2H), 6.94 (d, J=2.8 Hz, 1H), 6.89 (d, J=10.0 Hz, 1H), 5.08-4.64 (m), 4.58-4.37 (m), 4.32-4.25 (m), 4.24-4.06 (m), 3.99-3.86 (m), 3.81-3.60 (m), 3.60-3.43 (m), 2.99-2.88 (m), 2.86-2.69 (m), 2.64 (s, 3H), 2.44-2.30 (m), 2.29-2.16 (m), 2.14-1.90 (m), 1.83 (s, 3H), 1.67 (s, 3H), 1.22-1.11 (m), 0.99 (s, 3H). ¹⁹F NMR (376 MHz, Methanol-d4) δ −77.20 (s), −77.83 (s), −96.85 (d, J=6.2 Hz), −97.01 (d, J=6.2 Hz), −114.38 (s). ³¹P NMR (162 MHz, Methanol-d4) δ −6.89 (s).

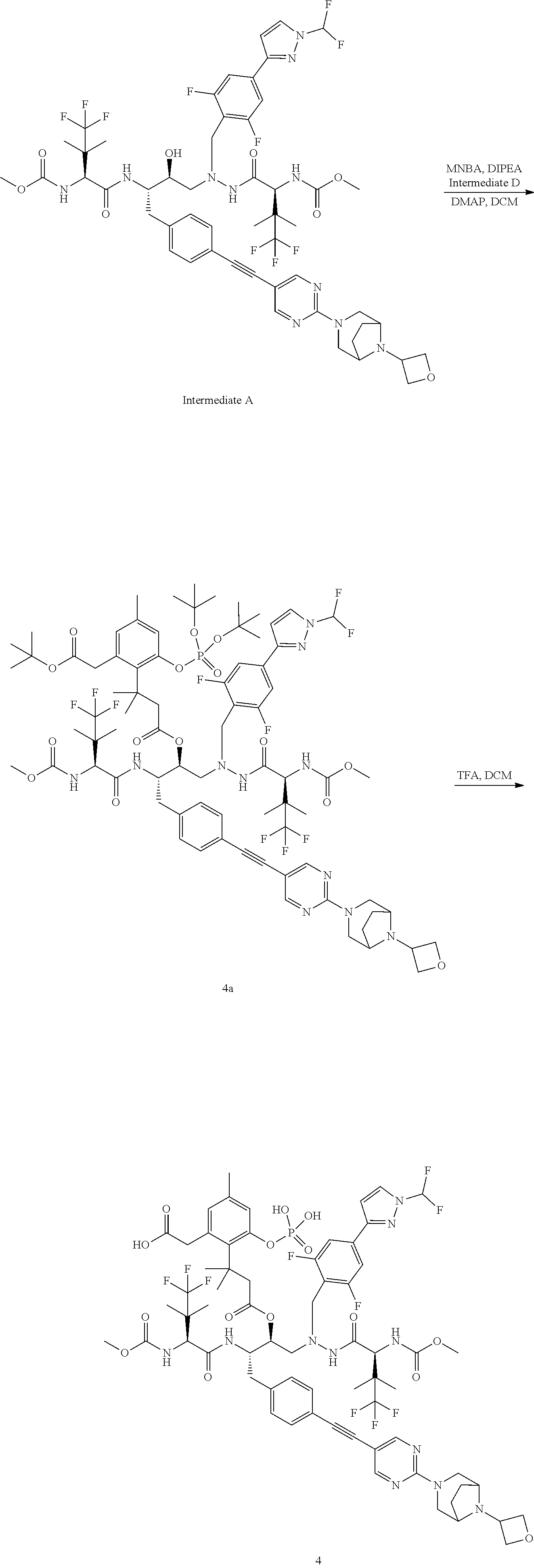

### Example 4

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-(tert-butoxy)-2-oxoethyl)-6-((di-tert-butoxyphosphoryl)oxy)-4-methylphenyl)-3-methylbutanoate (4a): To a mixture of Intermediate D (8.32 mmol), 2-methyl-6-nitrobenzoic anhydride (16.6 mmol), and DMAP (6.4 mmol) was added DCM (64 mL) followed by DIPEA (38.4 mmol), and then Intermediate A (6.4 mmol). The reaction was stirred at rt overnight. The reaction was then quenched with saturated aqueous ammonium chloride (32 mL) and water (32 mL). The organics were separated, and aqueous layer extracted with EtOAc (3×70 mL). The combined organic extracts were then washed with saturated aqueous sodium bicarbonate (5×), dried over Na₂SO₄, filtered, and concentrated. The crude material was triturated with EtOAc/hexanes to provide the title compound 4a. MS (m/z): 1653.2 [M+H]⁺.

Synthesis of 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetic acid (4): To a solution of 4a (1.82 mmol) in DCM (18.2 mL) was added TFA (5.97 mL). The reaction mixture was stirred at rt overnight, then concentrated and azeotroped with toluene (3×). The residue was triturated with EtOAc/hexanes to provide 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetic acid 4. m/z): 1485.2 [M+H]⁺. ¹H NMR (400 MHz, Methanol-d4) δ 8.53 (s, 2H), 8.10 (d, J=2.7 Hz, 1H), 7.72-7.36 (m), 7.32-7.25 (m), 7.25-7.22 (m), 7.11-7.03 (m), 6.94 (d, J=2.7 Hz, 1H), 6.91 (d, J=10.7 Hz, 1H), 6.64 (s, 1H), 5.01-4.93 (m), 4.84-4.71 (m), 4.52 (s, 1H), 4.38-4.09 (m), 3.93 (d, J=13.3 Hz, 1H), 3.77-3.63 (m), 3.55-3.39 (m), 2.97-2.87 (m), 2.83-2.70 (m), 2.35-2.16 (m), 2.06 (s, 3H), 2.06-1.91 (m), 1.77 (s, 3H), 1.62 (s, 3H), 1.19 (s, 3H), 1.18 (s, 3H), 1.16 (s, 3H), 1.02 (s, 3H). ¹⁹F NMR (376 MHz, Methanol-d4) δ −75.80 (s), −76.41 (s), −95.45 (d, J=6.8 Hz), −95.61 (d, J=6.8 Hz), −112.97 (s). P NMR (162 MHz, Methanol-d4) δ −7.05 (s).

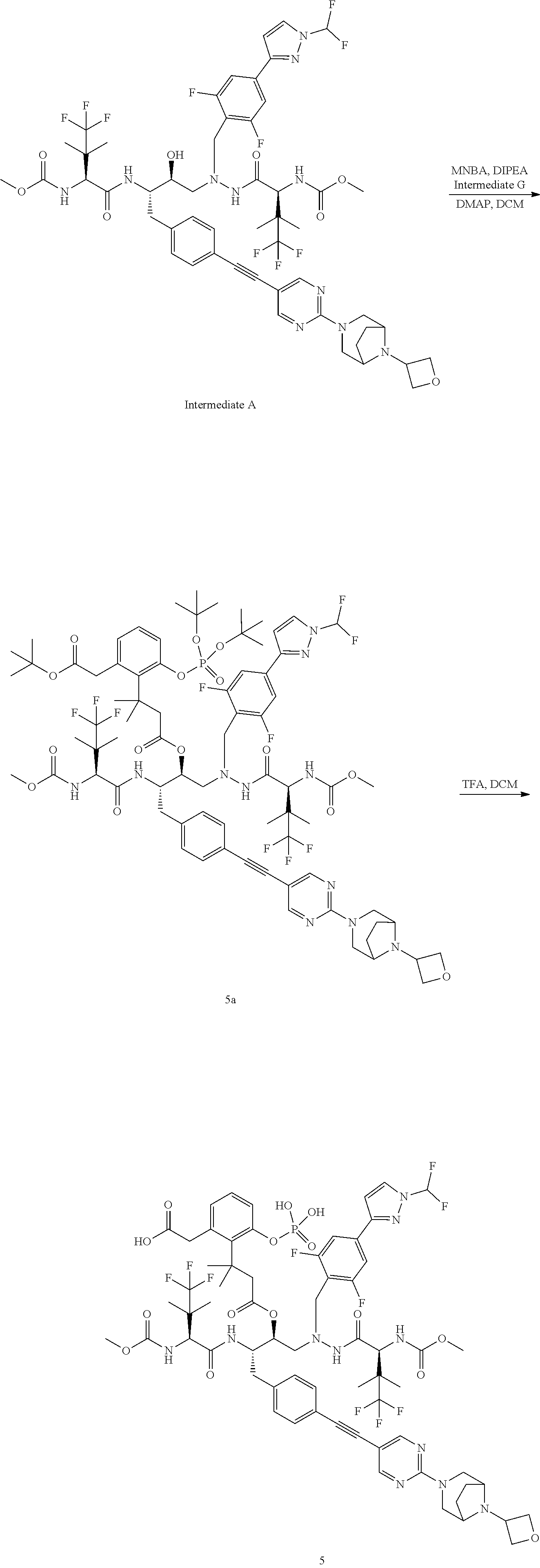

### Example 5

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-(tert-butoxy)-2-oxoethyl)-6-((di-tert-butoxyphosphoryl)oxy)phenyl)-3-methylbutanoate (5a): To a mixture of Intermediate G (0.304 mmol), 2-methyl-6-nitrobenzoic anhydride (0.607 mmol), and DMAP (0.234 mmol) was added DCM (2.5 mL) followed by DIPEA (1.4 mmol), and then Intermediate A (0.234 mmol). The reaction was stirred at rt overnight. The reaction was then quenched with saturated aqueous ammonium chloride. The organics were separated, and aqueous layer extracted with EtOAc (×3). The combined organic extracts were dried over Na₂SO₄, filtered, and concentrated. The crude material was purified by reverse-phase HPLC (eluent: water/MeCN 0.1% TFA) to provide the title compound 5a.

Synthesis of 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetic acid (5): To a solution of 5a (0.111 mmol) in DCM (1.11 mL) was added TFA (0.370 mL). The reaction mixture was stirred at rt for 2 h, then concentrated. The residue was purified by reverse-phase HPLC (eluent: water/MeCN 0.1% TFA) to provide 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetic acid 5. m/z): 1470.2 [M+H]⁺. ¹H NMR (400 MHz, Methanol-d4) δ 8.52 (s, 2H), 8.10 (d, J=2.7 Hz, 1H), 7.73-7.35 (m), 7.28 (d, J=7.8 Hz, 2H), 7.09 (d, J=7.9 Hz, 2H), 7.04 (t, J=7.9 Hz, 1H), 6.98-6.88 (m), 6.88-6.76 (m), 5.04-4.92 (m), 4.83-4.64 (m), 4.60-4.36 (m), 4.28 (d, J=10.1 Hz, 1H), 4.25-4.07 (m), 3.91 (d, J=13.3 Hz, 1H), 3.79 (d, J=18.2 Hz, 1H), 3.72 (s, 3H), 3.66 (s, 3H), 3.54-3.44 (m), 3.04-2.84 (m), 2.71-2.58 (m), 2.48-2.34 (m), 2.33-2.12 (m), 2.06-1.92 (m), 1.76 (s, 3H), 1.65 (s, 3H), 1.23-1.04 (m), 0.95 (s, 3H). ¹⁹F NMR (376 MHz, DMSO-d6) δ −74.85 (s), −75.35 (s), −95.04 (s), −95.09 (s), −112.73 (s). ³¹P NMR (162 MHz, Methanol-d4) δ −6.92.

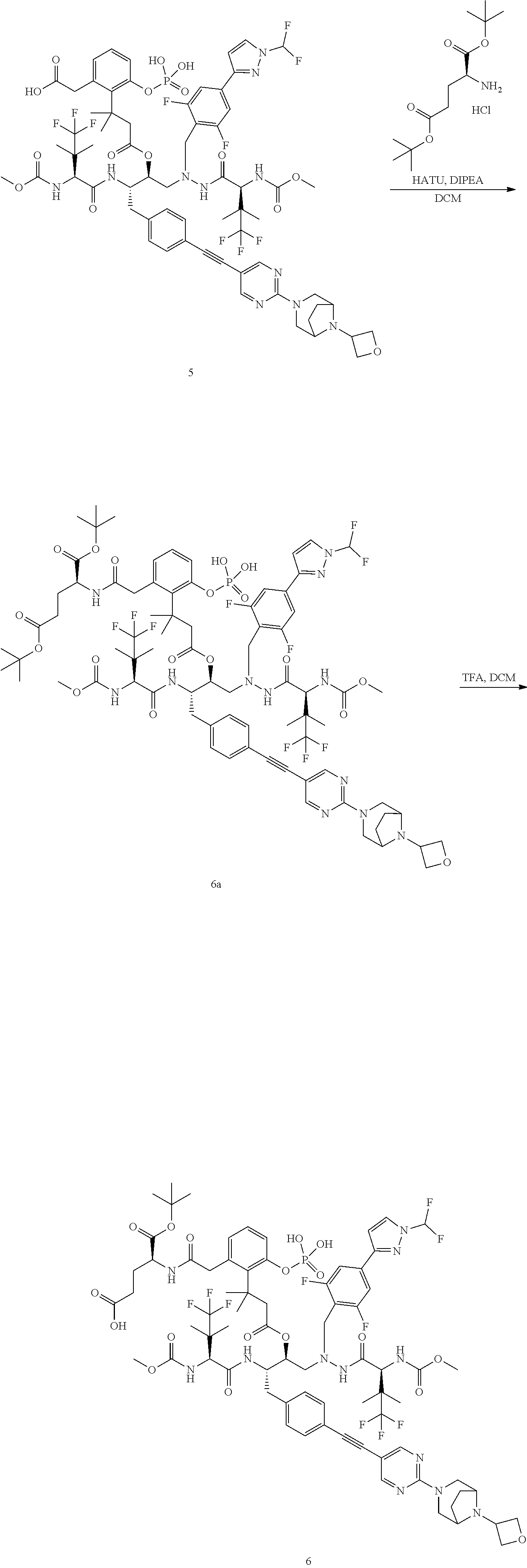

### Example 6

Synthesis of di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-glutamate (6a): To a solution of 5 (0.032 mmol) in DCM (0.32 mL) was added L-glutamic acid di-tert-butyl ester hydrochloride (0.079 mmol), HATU (0.095 mmol), and DIPEA (0.189 mmol). The reaction was stirred at rt for 18 h, then concentrated and purified by reverse-phase HPLC (eluent: water/MeCN 0.1% TFA) to provide the title compound 6a. MS (m/z): 856.6 [M+2H]²⁺.

Synthesis of (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-glutamic acid (6): To a solution of 6a (0.043 mmol) in DCM (0.43 mL) was added TFA (0.14 mL). The reaction was stirred at rt for 3 h, then concentrated. The residue was purified by reverse-phase HPLC (eluent: water/MeCN 0.1% TFA) to provide (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-glutamic acid (6). m/z): 1599.3 [M+H]⁺. ¹H NMR (400 MHz, Methanol-d4) δ 8.52 (s, 2H), 8.10 (d, J=2.7 Hz, 1H), 7.73-7.36 (m), 7.29 (d, J=7.9 Hz, 2H), 7.16-7.01 (m), 6.99-6.86 (m), 5.01-4.93 (m), 4.82-4.66 (m), 4.58-4.48 (m), 4.46-4.35 (m), 4.33-4.24 (m), 4.23-4.09 (m), 3.99-3.79 (m), 3.77-3.68 (m), 3.65 (s, 3H), 3.53-3.43 (m), 3.02-2.85 (m), 2.78-2.61 (m), 2.46-2.34 (m), 2.32-2.11 (m), 2.06-1.92 (m), 1.76 (s, 3H), 1.66 (s, 3H), 1.24-1.02 (m), 0.95 (s, 3H). ¹⁹F NMR (376 MHz, DMSO-d6) δ −74.84 (s), −75.35 (s), −95.04 (s), −95.09 (s), −112.75 (s). ³¹P NMR (162 MHz, Methanol-d4) δ −6.88.

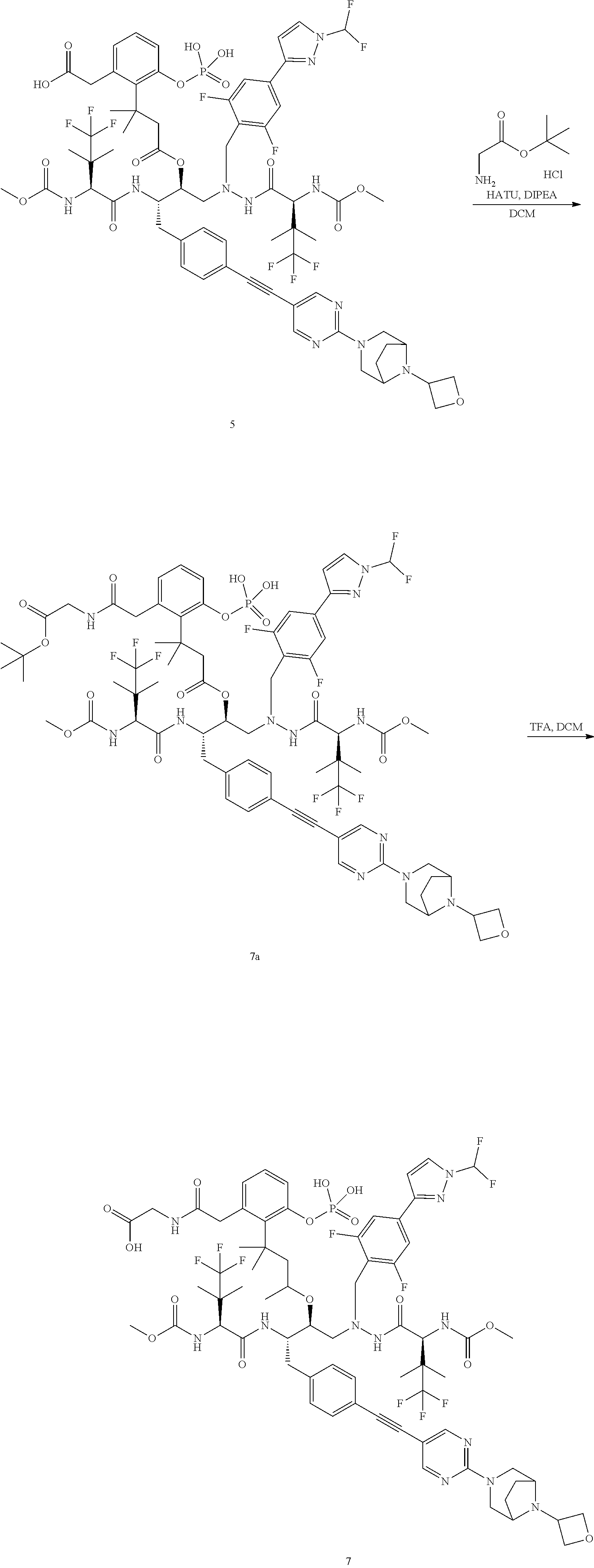

### Example 7

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((2-(tert-butoxy)-2-oxoethyl)amino)-2-oxoethyl)-6-(phosphonooxy)phenyl)-3-methylbutanoate (7a): (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((2-(tert-butoxy)-2-oxoethyl)amino)-2-oxoethyl)-6-(phosphonooxy)phenyl)-3-methylbutanoate (7a) was synthesized using the method described in Step 1 of Example 6 substituting glycine tert-butyl ester hydrochloride for L-glutamic acid di-tert-butyl ester hydrochloride. MS (m/z): 792.5 [M+2H]²⁺.

Synthesis of (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)glycine (7): (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)glycine (7) was synthesized using the method described in Step 2 of Example 6, substituting 7a for 6a. m/z): 1527.3 [M+H]⁺. ¹H NMR (400 MHz, Methanol-d4) δ 8.52 (s, 2H), 8.10 (d, J=2.8 Hz, 1H), 7.72-7.35 (m), 7.29 (d, J=7.8 Hz, 2H), 7.16-7.04 (m), 6.99-6.93 (m), 6.90 (d, J=10.2 Hz, 1H), 5.07-4.92 (m), 4.83-4.62 (m), 4.56-4.35 (m), 4.33-4.22 (m), 4.22-4.09 (m), 4.01-3.81 (m), 3.81-3.69 (m), 3.65 (s, 3H), 3.54-3.44 (m), 3.09-2.87 (m), 2.80-2.61 (m), 2.52-2.38 (m), 2.38-2.27 (m), 2.27-2.15 (m), 2.09-1.90 (m), 1.76 (s, 3H), 1.66 (s, 3H), 1.25-1.05 (m), 0.94 (s, 3H). ¹⁹F NMR (376 MHz, DMSO-d6) δ −74.84 (s), −75.34 (s), −95.04 (s), −95.09 (s), −112.74 (s). ³¹P NMR (162 MHz, Methanol-d4) δ −6.70.

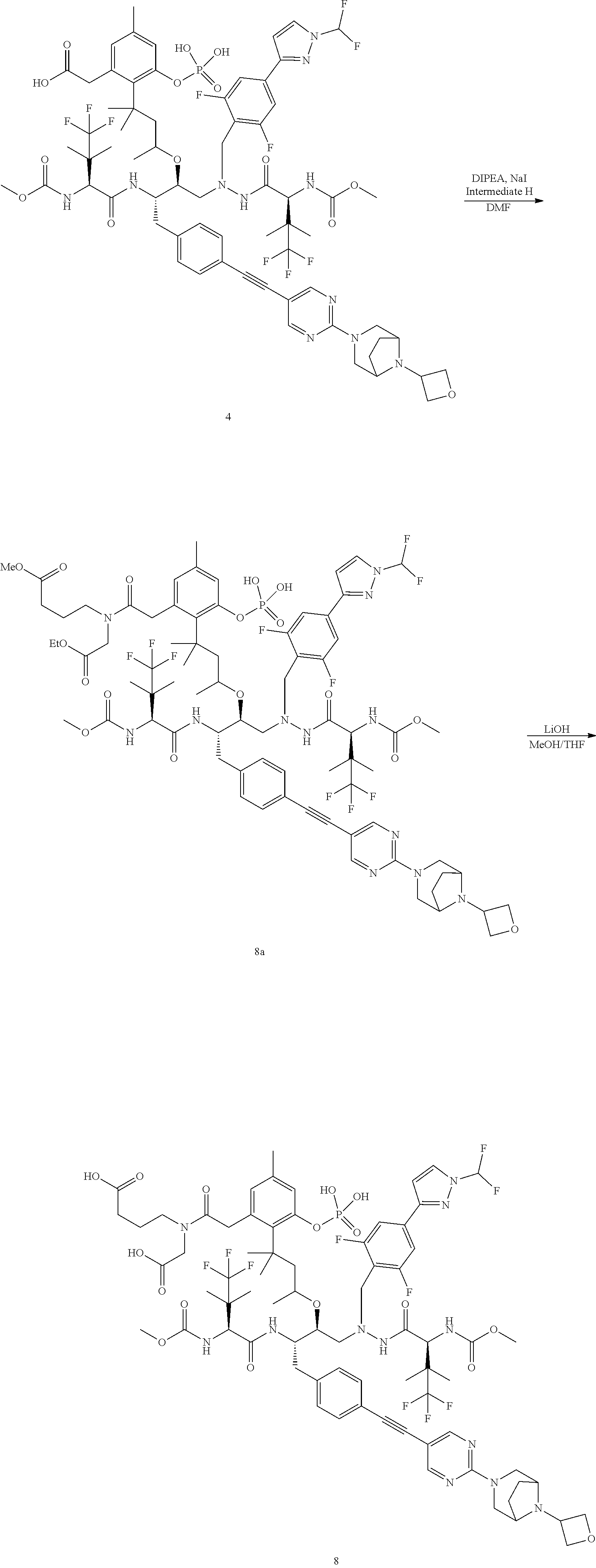

### Example 8

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((2-ethoxy-2-oxoethyl)(4-methoxy-4-oxobutyl)amino)-2-oxoethyl)-4-methyl-6-(phosphonooxy)phenyl)-3-methylbutanoate (8a): To a solution of 4 (0.097 mmol) in DCM (0.97 mL) was added Intermediate H (0.242 mmol), HATU (0.116 mmol), and DIPEA (0.291 mmol). The reaction was stirred at rt for 18 h, then concentrated and purified by reverse-phase HPLC (eluent: water/MeCN 0.1% TFA) to provide the title compound 8a.

Synthesis of 4-(N-(carboxymethyl)-2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)butanoic acid (8): To a solution of Intermediate 8a in MeOH/THF (1:1, 1.0 mL) was added aqueous LiOH (1.0M, 0.15 mL). The reaction was stirred overnight, at which time additional aqueous LiOH (1.0M, 0.3 mL) was added. Upon completion of the reaction, aqueous AcOH (0.1M) was added to neutralize the reaction, and the mixture was directly purified by reverse-phase HPLC (eluent: water/MeCN 0.1% TFA) to provide the title compound 8. m/z): 1627.5 [M+H]⁺. ¹H NMR (400 MHz, Methanol-d4) δ 8.52 (s, 2H), 8.10 (d, J=2.8 Hz, 1H), 7.73-7.35 (m), 7.28 (d, J=7.8 Hz, 2H), 7.23 (s, 1H), 7.14-7.03 (m), 7.00-6.86 (m), 6.70 (s, 1H), 6.59-6.41 (m), 4.99-4.92 (m), 4.78-4.66 (m), 4.52 (s, 1H), 4.40-4.25 (m), 4.25-4.18 (m), 4.18-4.08 (m), 4.05-3.86 (m), 3.85-3.76 (m), 3.72 (s, 3H), 3.69-3.59 (m), 3.57-3.37 (m), 3.00-2.80 (m), 2.80-2.62 (m), 2.46-2.27 (m), 2.27-2.17 (m), 2.16-1.81 (m), 1.78-1.65 (m), 1.65-1.50 (m), 1.24-1.05 (m), 1.03-0.89 (m). ¹⁹F NMR (376 MHz, Methanol-d4) δ −77.18 (d, J=11.9 Hz), −77.77 (d, J=8.9 Hz), −96.83-−97.04 (m), −114.31. ³¹P NMR (162 MHz, Methanol-d4) δ −7.04 (d, J=9.5 Hz).

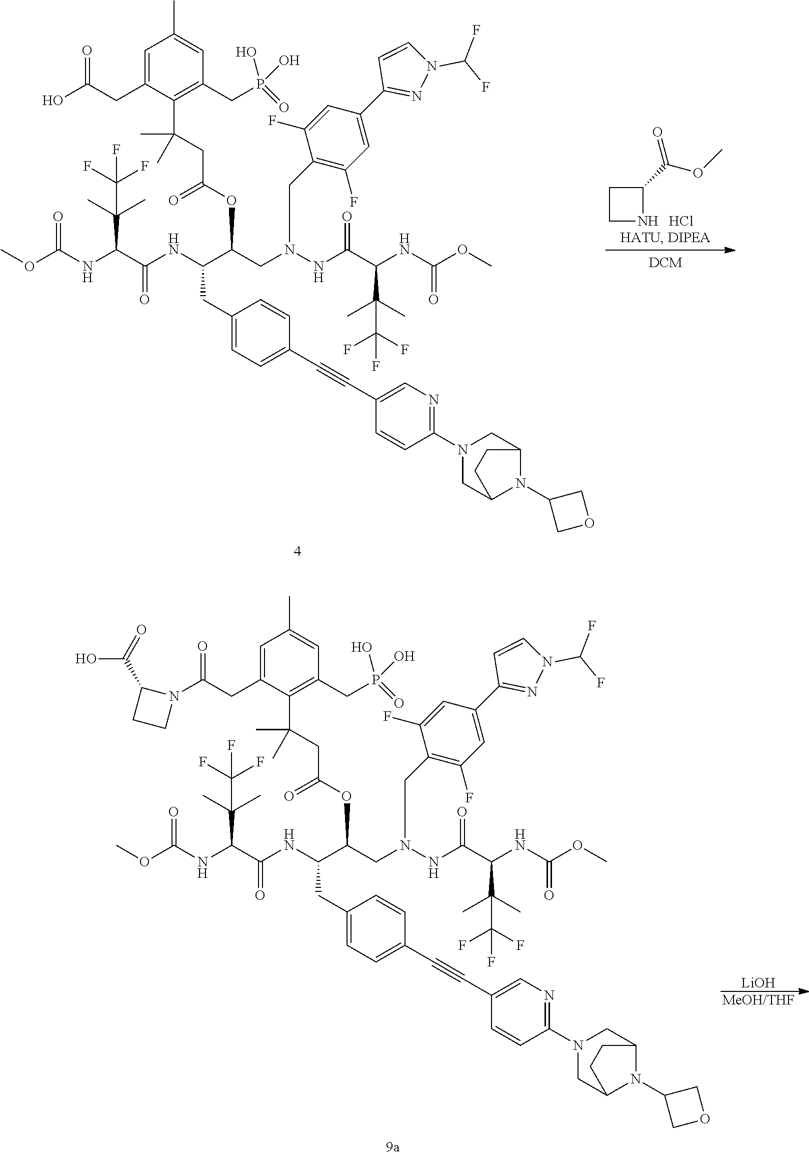

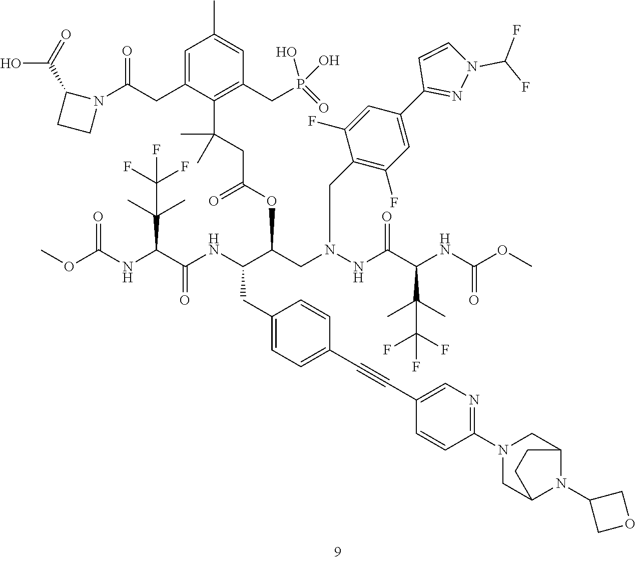 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC[C@@H]2C(=O)O)cc(C)cc1CP(=O)(O)O)C(C)(C)C(F)(F)F]

### Example 9

Synthesis of methyl (2R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)azetidine-2-carboxylate (9a): methyl (2R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)azetidine-2-carboxylate (9a) was synthesized using the method described in Step 1 of Example 8 substituting methyl (2R)-azetidine-2-carboxylate hydrochloride for Intermediate H. MS (m/z): 791.5 [M+2H]²⁺.

Synthesis of (2R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)azetidine-2-carboxylic acid (9): (2R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)azetidine-2-carboxylic acid (9) was synthesized using the method described in Step 2 of Example 6, substituting 9a for 8a. m/z): 1567.2 [M+H]⁺. ¹H NMR (400 MHz, Methanol-d4) δ 8.53 (s, 2H), 8.14-8.08 (m), 7.72-7.35 (m), 7.29 (d, J=7.5 Hz, 2H), 7.23 (d, J=7.8 Hz, 1H), 7.14-7.04 (m), 6.94 (d, J=2.7 Hz, 1H), 6.92-6.83 (m), 6.71-6.53 (m), 5.01-4.92 (m), 4.81-4.66 (m), 4.58-4.42 (m), 4.41-3.86 (m), 3.75-3.68 (m), 3.68-3.64 (m), 3.64-3.37 (m), 3.04-2.59 (m), 2.37-2.15 (m), 2.14-1.94 (m), 1.84-1.66 (m), 1.62 (s, 3H), 1.24-1.05 (m), 1.04-0.84 (m). ¹⁹F NMR (376 MHz, Methanol-d4) δ −77.16 (d, J=12.2 Hz), −77.84 (m), −96.95 (d, J=6.1 Hz), −114.38 (s). ³¹P NMR (162 MHz, Methanol-d4) δ −6.97 (d, J=5.1 Hz).

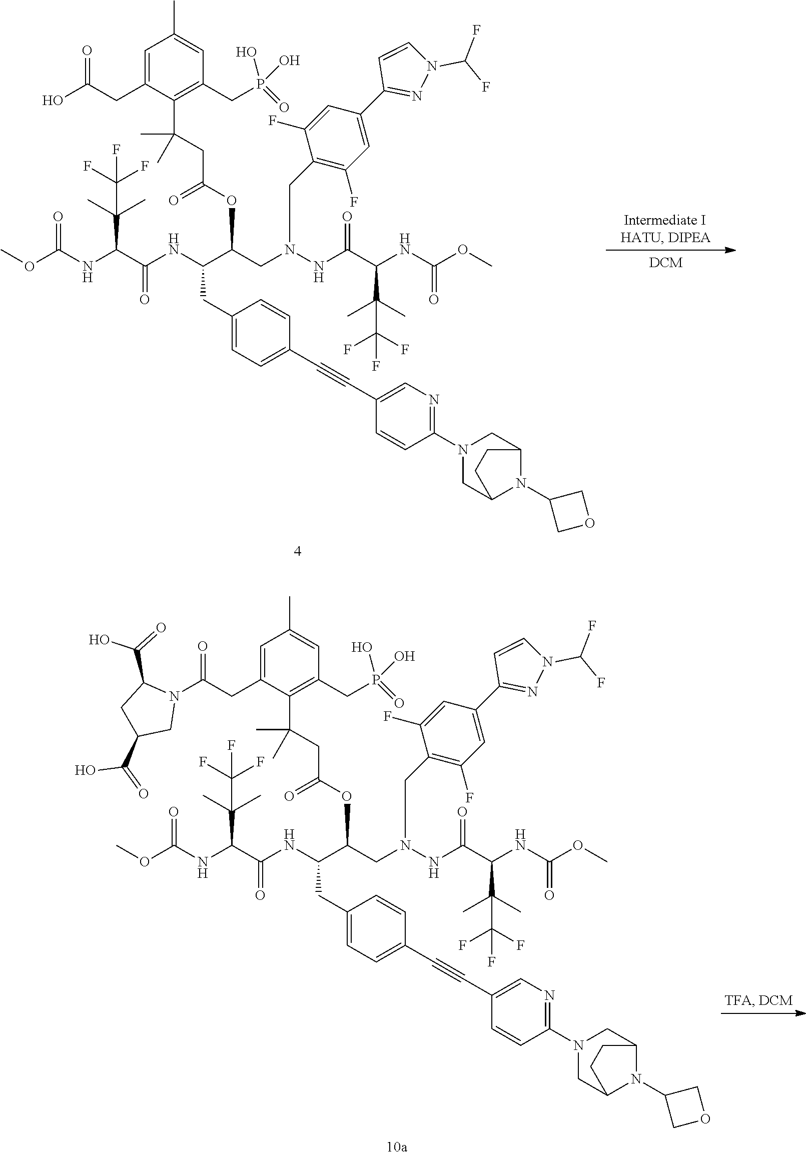 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1CP(=O)(O)O)C(C)(C)C(F)(F)F ; COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2C[C@@H](C(=O)O)C[C@H]2C(=O)O)cc(C)cc1CP(=O)(O)O)C(C)(C)C(F)(F)F]

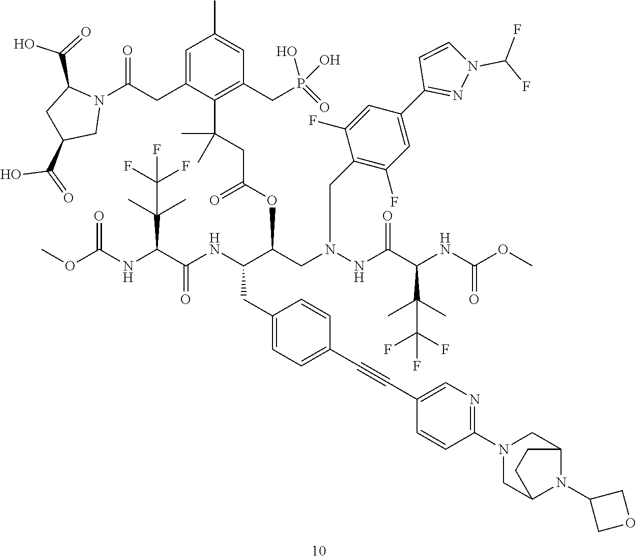 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2C[C@@H](C(=O)O)C[C@H]2C(=O)O)cc(C)cc1CP(=O)(O)O)C(C)(C)C(F)(F)F]

### Example 10

Synthesis of di-tert-butyl (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)pyrrolidine-2,4-dicarboxylate (10a): To a solution of 4 (0.0957 mmol) in DCM (0.96 mL) was added Intermediate I (0.239 mmol), HATU (0.115 mmol), and DIPEA (0.287 mmol). The reaction was stirred at rt for 18 h, then concentrated and purified by reverse-phase HPLC (eluent: water/MeCN 0.1% TFA) to provide the title compound 10a. MS (m/z): 869.5 [M+2H]²⁺.

Synthesis of (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)pyrrolidine-2,4-dicarboxylic acid (10): To a solution of 10a (0.0683 mmol) in DCM (0.68 mL) was added TFA (0.23 mL). The reaction was stirred at rt for 3 h, at which point another 0.2 mL TFA was added. After 1.5 h, the reaction was concentrated and purified by reverse-phase HPLC (eluent: water/MeCN 0.1% TFA) to provide the title compound 10. m/z): 1625.1 [M+H]⁺. ¹H NMR (400 MHz, Methanol-d4) δ 8.53 (d, J=1.2 Hz, 2H), 8.11 (d, J=2.7 Hz, 1H), 7.72-7.37 (m), 7.28 (d, J=7.8 Hz, 2H), 7.24 (s, 1H), 7.16-7.05 (m), 6.94 (d, J=2.7 Hz, 1H), 6.91 (d, J=9.9 Hz, 1H), 6.76-6.53 (m), 5.08-4.92 (m), 4.79-4.66 (m), 4.60-4.42 (m), 4.40-4.08 (m), 4.05-3.86 (m), 3.86-3.75 (m), 3.72 (s, 3H), 3.69-3.59 (m), 3.57-3.42 (m), 3.27-3.21 (m), 3.01-2.84 (m), 2.85-2.67 (m), 2.67-2.53 (m), 2.40-2.14 (m), 2.12-1.93 (m), 1.78-1.66 (m), 1.62 (s, 3H), 1.25-1.09 (m), 1.09-0.93 (m). ¹⁹F NMR (376 MHz, DMSO-d6) δ −74.82 (s), −75.33 (s), −95.07 (d, J=20.1 Hz), −112.75 (s). ³¹P NMR (162 MHz, DMSO-d6) δ −8.01.

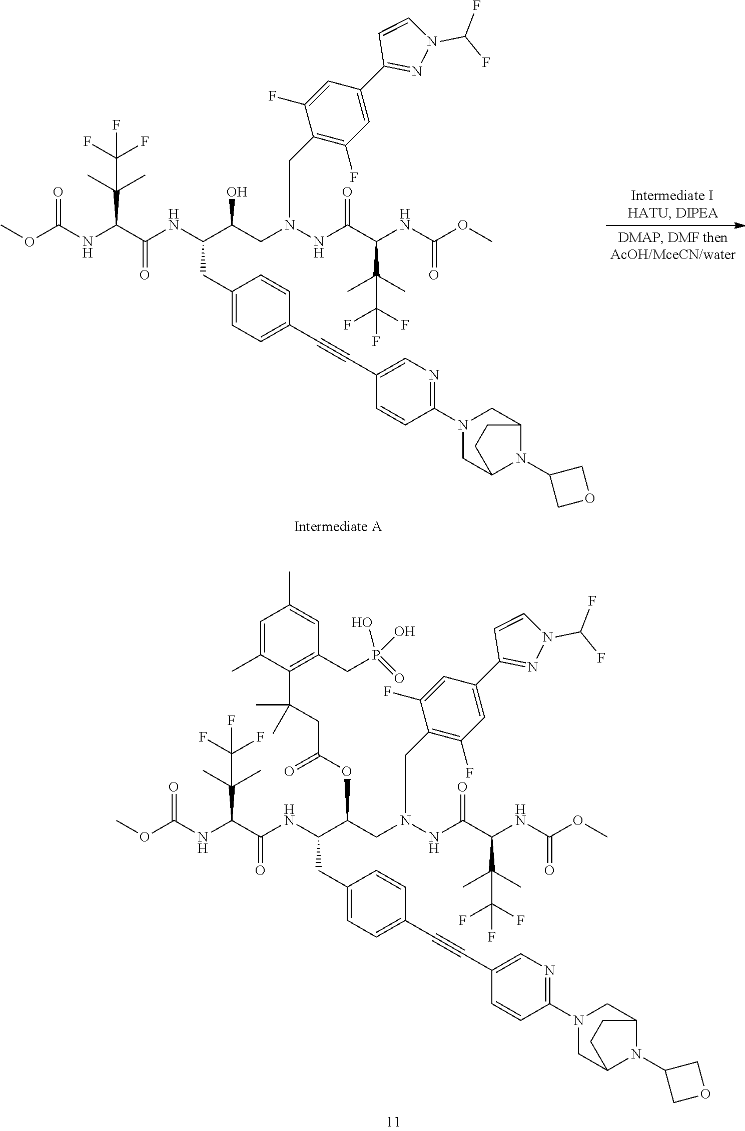 [SMILES: COC(=O)N[C@H](C(=O)N[C@@H](Cc1ccc(C#Cc2ccc(N3CC4CCC(C3)N4C3COC3)nc2)cc1)[C@@H](O)CN(Cc1c(F)cc(-c2ccn(C(F)F)n2)cc1F)NC(=O)[C@@H](NC(=O)OC)C(C)(C)C(F)(F)F)C(C)(C)C(F)(F)F ; COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(C)cc(C)cc1CP(=O)(O)O)C(C)(C)C(F)(F)F]

### Example 11

HATU (0.13 mmol) was combined with 3-(2-ditert-butoxyphosphoryloxy-4,6-dimethyl-phenyl)-3-methyl-butanoic acid Intermediate J (0.13 mmol) in DMF (0.3 mL). After 5 min, Intermediate A (0.086 mmol) and DIPEA (0.26 mmoL) were added and the mixture was stirred at 50° C. After 3 hours, DMAP (2 equiv) was added and the mixture was stirred overnight at 50° C. After 18 hours, the reaction diluted with EtOAc (3 mL), rinsed with sat. aq NH₄Cl (2 mL), then sat. aq NaHCO₃ (2 mL), dried over Na₂SO₄, filtered and concentrated to dryness. The residue was redissolved in MeCN:water:AcOH (1:1:2, 1 mL) and stirred at 50° C. After 4 hours, the reaction was concentrated and purified by RP-HPLC (MeCN/H₂O with 0.1% TFA) to afford the title compound 11. MS (m/z): 1440.1 [M+H]⁺. ¹H NMR (400 MHz, Methanol-d4) δ 8.53 (s, 2H), 8.10 (d, J=2.8 Hz, 1H), 7.72-7.35 (m), 7.28 (d, J=7.9 Hz, 2H), 7.15 (s, 1H), 7.08 (d, J=8.0 Hz, 2H), 6.98-6.86 (m), 6.73-6.66 (m), 6.59 (s, 1H), 4.96 (t, J=7.6 Hz, 2H), 4.83-4.69 (m), 4.53 (s, 1H), 4.36 (s, 1H), 4.32-4.25 (m), 4.20 (d, J=13.4 Hz, 1H), 4.14 (s, 2H), 3.93 (d, J=13.4 Hz, 1H), 3.72 (s, 3H), 3.66 (s, 3H), 3.57-3.41 (m), 3.01-2.87 (m), 2.84-2.71 (m), 2.66 (d, J=15.0 Hz, 1H), 2.53 (s, 3H), 2.29-2.15 (m), 2.10-1.95 (m), 1.81 (s, 3H), 1.62 (s, 3H), 1.27-1.08 (m), 1.06-0.90 (m). ¹⁹F NMR (376 MHz, Methanol-d4) δ −77.19 (s), −77.81 (s), −96.94 (d, J=6.7 Hz), −114.38 (s).

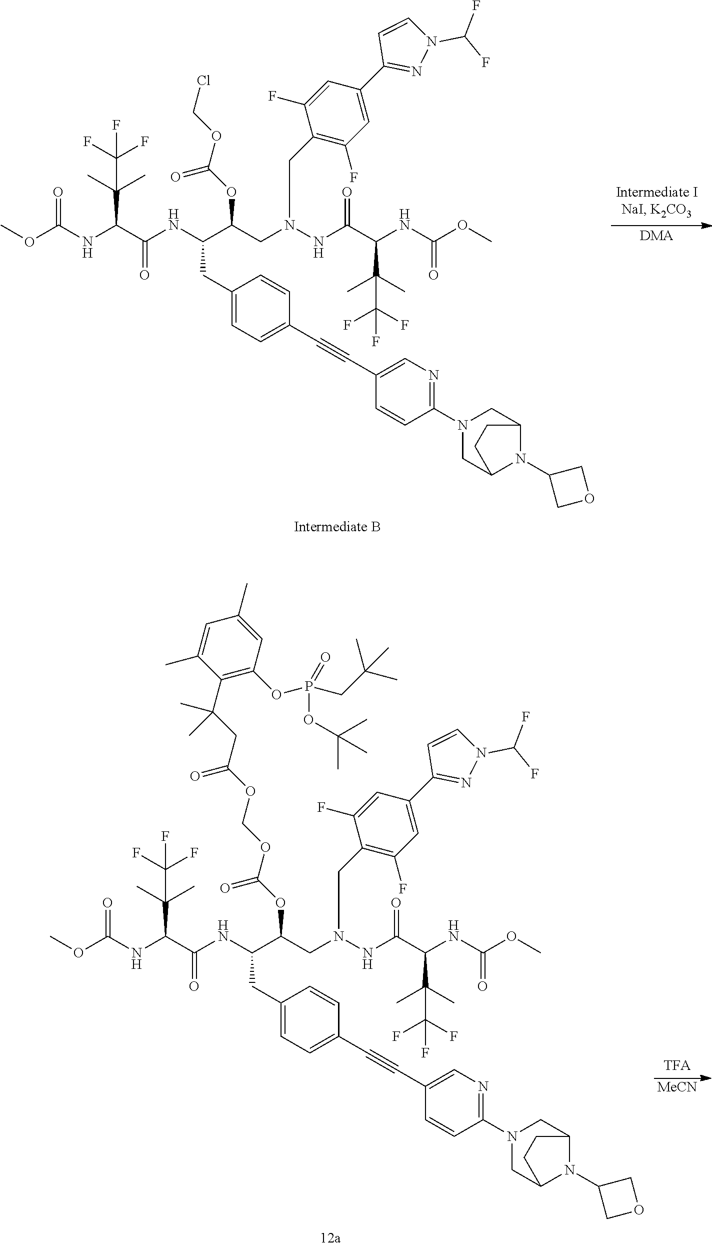

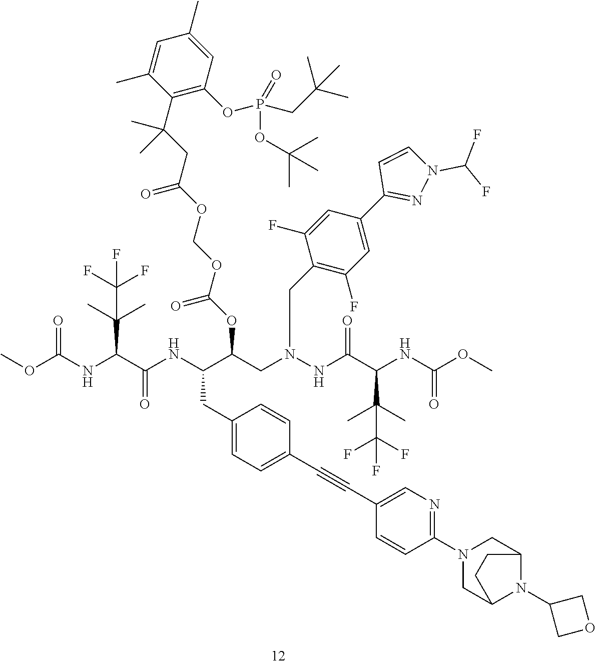 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(C)cc(C)cc1OP(=O)(CC(C)(C)C)OC(C)(C)C)C(C)(C)C(F)(F)F]

### Example 12

Synthesis of (5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2-((di-tert-butoxyphosphoryl)oxy)-4,6-dimethylphenyl)-3-methylbutanoate (12): Intermediate B (0.036 mmol) was combined with 3-(2-ditert-butoxyphosphoryloxy-4,6-dimethyl-phenyl)-3-methyl-butanoic acid Intermediate J (0.041 mmol), K₂CO₃ (0.11 mmol), and NaI (0.072 mmol) in DMA (0.3 mL). The reaction was stirred at 40° C. for 1 hour, then at 60° C. for 2 hours. The mixture was cooled to rt, filtered, diluted with aq. TFA and purified by RP-HPLC (MeCN/H₂O with 0.1% TFA) to provide (5S,10S)-8-(4-(1-(difluoromethyl)-1H-pyrazol-3-yl)-2,6-difluorobenzyl)-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2-((di-tert-butoxyphosphoryl)oxy)-4,6-dimethylphenyl)-3-methylbutanoate 12a. MS (m/z): 1627.3 [M+H]⁺.

Synthesis of (5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2,4-dimethyl-6-(phosphonooxy)phenyl)-3-methylbutanoate (12): Intermediate 12a was then treated with TFA (0.1 mL) in MeCN (0.1 mL) for 3 hours at rt, then diluted with water and lyophilized to provide (5S,10S)-8-(4-(1-(difluoromethyl)-1H-pyrazol-3-yl)-2,6-difluorobenzyl)-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2,4-dimethyl-6-(phosphonooxy)phenyl)-3-methylbutanoate 12. MS (m/z): 1515.4 [M+H]⁺. ¹H NMR (400 MHz, Methanol-d4) δ 8.55 (s, 2H), 8.12 (d, J=2.7 Hz, 1H), 7.55 (t, J=59.8 Hz, 1H), 7.45 (d, J=8.2 Hz, 2H), 7.36 (d, J=8.0 Hz, 2H), 7.24 (d, J=8.0 Hz, 2H), 7.12 (s, 1H), 6.94 (d, J=2.7 Hz, 1H), 6.70 (s, 1H), 5.74 (d, J=5.9 Hz, 1H), 5.63 (d, J=5.9 Hz, 1H), 4.98 (t, J=7.6 Hz, 2H), 4.86-4.76 (m), 4.61 (s, 1H), 4.50-4.44 (m), 4.35-4.26 (m), 4.16 (s, 2H), 4.10-4.04 (m), 3.71 (s, 3H), 3.68 (s, 3H), 3.51 (s, 1H), 3.47 (s, 1H), 3.25 (dd, J=13.5, 5.7 Hz, 1H), 3.12 (s, 2H), 3.03-2.86 (m), 2.79-2.69 (m), 2.53 (s, 3H), 2.29-2.21 (m), 2.20 (s, 3H), 2.04-1.98 (m), 1.88 (s, 3H), 1.64 (s, 6H), 1.22 (d, J=1.1 Hz, 3H), 1.18 (s, 3H), 1.15 (s, 3H), 1.07 (s, 3H).

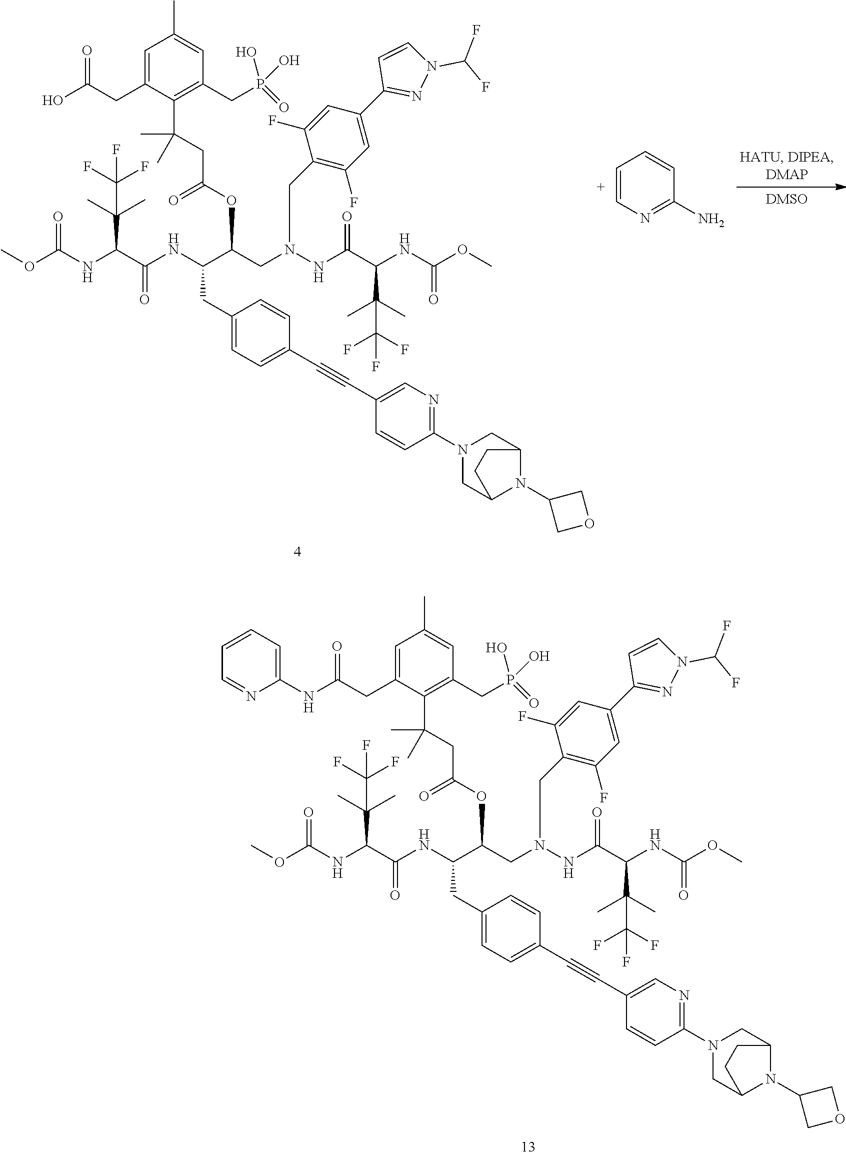 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1CP(=O)(O)O)C(C)(C)C(F)(F)F ; Nc1ccccn1 ; COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccccn2)cc(C)cc1CP(=O)(O)O)C(C)(C)C(F)(F)F]

### Example 13

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-oxo-2-(pyridin-2-ylamino)ethyl)-6-(phosphonooxy)phenyl)butanoate (13): To a solution of 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetic acid 4 (0.03 mmol) in DMSO (1.0 mL) was added HATU (0.12 mmol), DIPEA (0.2 mmol), 2-aminopyridine (0.12 mmol), and 4-dimethylaminopyridine (0.009 mmol). The reaction mixture was stirred at rt for 4 hours. The crude reaction mixture was directly purified via reverse-phase HPLC (eluent: water/MeCN containing 0.1% TFA) to provide (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-oxo-2-(pyridin-2-ylamino)ethyl)-6-(phosphonooxy)phenyl)butanoate 13. MS (m/z): 1560.0 [M+H]⁺. ¹H NMR (400 MHz, Methanol-d4) δ 8.55 (s, 2H), 8.32 (d, J=5.1 Hz, 1H), 8.11 (d, J=2.7 Hz, 1H), 8.03 (t, J=8.0 Hz, 1H), 7.90 (d, J=8.5 Hz, 1H), 7.72-7.38 (m), 7.37-7.20 (m), 7.18-7.03 (m), 6.94 (d, J=2.8 Hz, 1H), 6.73 (s, 1H), 4.98 (t, J=7.5 Hz, 3H), 4.74 (s, 1H), 4.45 (d, J=17.7 Hz, 1H), 4.38-4.27 (m), 4.26-4.14 (m), 4.06 (d, J=17.8 Hz, 1H), 3.94 (d, J=13.4 Hz, 1H), 3.73 (s, 3H), 3.69 (s, 3H), 3.64-3.45 (m), 3.10-2.92 (m), 2.82 (d, J=15.2 Hz, 2H), 2.40 (dd, J=13.5, 5.1 Hz, 1H), 2.32-2.21 (m), 2.16-1.97 (m), 1.81 (s, 3H), 1.66 (s, 3H), 1.31 (s, 1H), 1.29-1.12 (m), 1.04 (s, 3H).

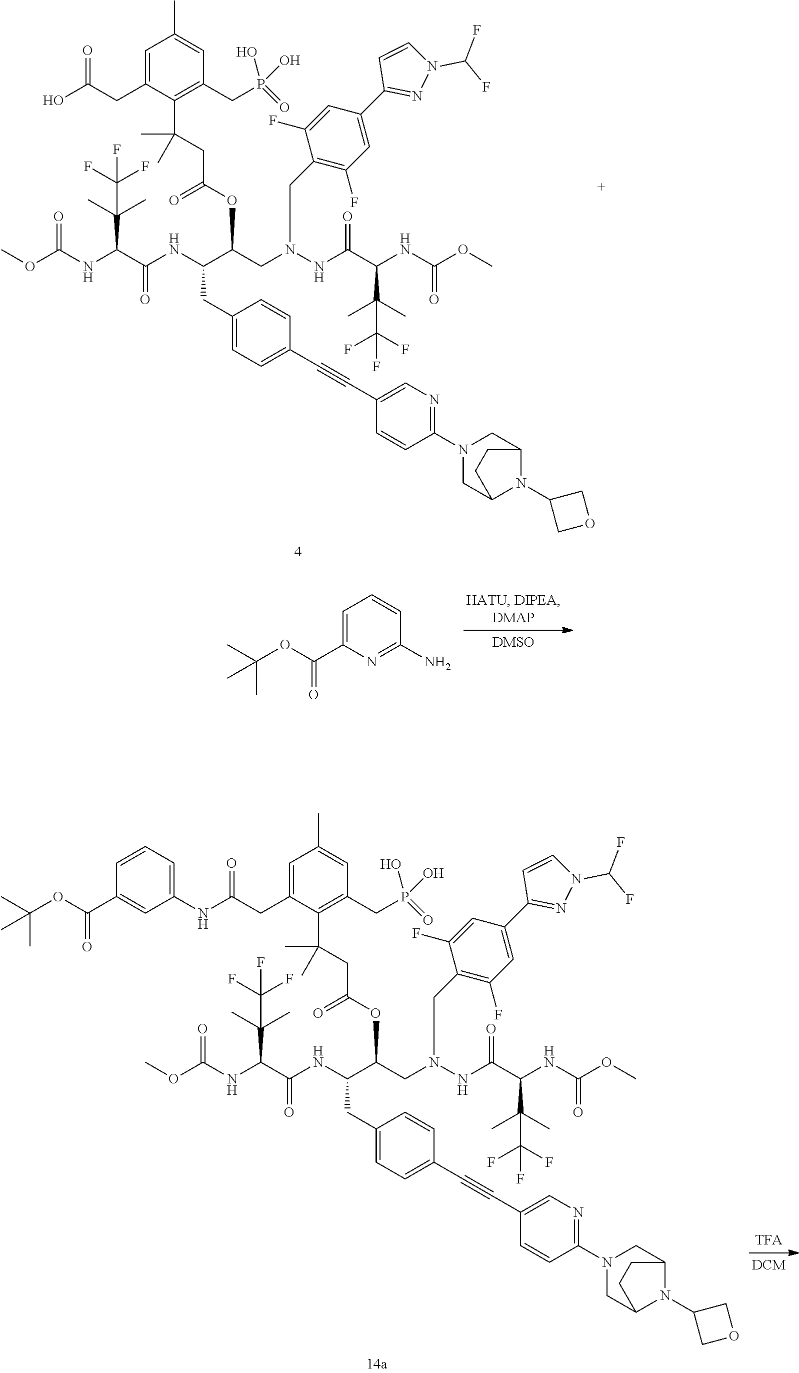

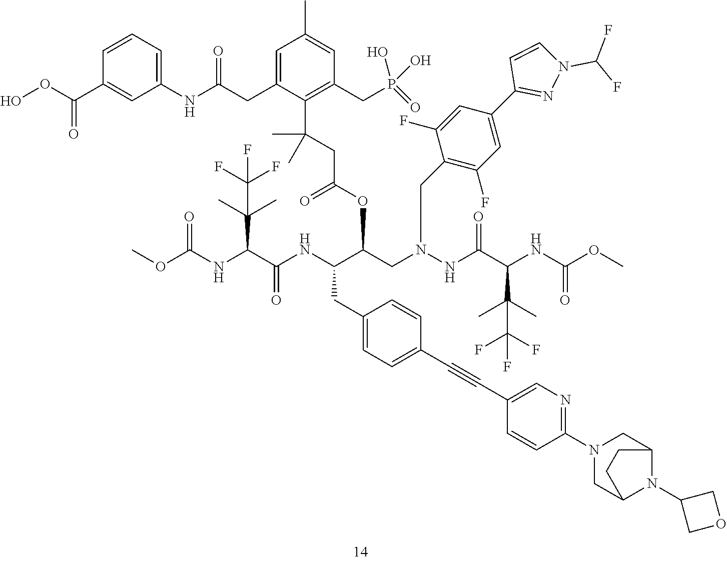 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(C(=O)OO)c2)cc(C)cc1CP(=O)(O)O)C(C)(C)C(F)(F)F]

### Example 14

Synthesis of tert-butyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoate (14a): To a solution of 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetic acid 4 (0.05 mmol) in DMSO (1.0 mL) was added HATU (0.15 mmol), DIPEA (0.2 mmol), tert-butyl 3-aminobenzoate (0.15 mmol), and 4-dimethylaminopyridine (0.009 mmol). The reaction mixture was stirred at rt for 4 hours. The crude reaction mixture was directly purified via reverse-phase HPLC (eluent: water/MeCN containing 0.1% TFA) to provide tert-butyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoate 14a. MS (m/z): 1660.5 [M+H]⁺.

Synthesis of 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoic acid (14): To a solution of tert-butyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoate 14a (0.04 mmol) in DCM (3 mL) was added TFA (1 mL). The reaction mixture was stirred for 90 mins at rt. The crude reaction mixture was concentrated under vacuum. The crude residue was purified by reverse-phase HPLC (eluent: water/MeCN containing 0.1% TFA) to provide 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoic acid 14. MS (m/z): 1604.1.1 [M+H]⁺. ¹H NMR (400 MHz, Methanol-d4) δ 8.54 (s, 2H), 8.19 (s, 1H), 8.11 (d, J=2.7 Hz, 1H), 7.87 (dd, J=8.1, 1.2 Hz, 1H), 7.74 (d, J=7.8 Hz, 1H), 7.72-7.36 (m), 7.29 (d, J=7.7 Hz, 3H), 7.10 (d, J=7.9 Hz, 2H), 7.00-6.86 (m), 6.79 (s, 1H), 4.98 (t, J=7.6 Hz, 2H), 4.80 (d, J=33.5 Hz, 3H), 4.30 (dd, J=13.0, 6.2 Hz, 3H), 4.25-4.09 (m), 3.99 (d, J=17.2 Hz, 1H), 3.90 (d, J=13.1 Hz, 1H), 3.74 (s, 3H), 3.67 (s, 3H), 3.60-3.42 (m), 3.11-2.98 (m), 2.94 (d, J=15.7 Hz, 1H), 2.84 (dd, J=13.2, 6.4 Hz, 1H), 2.33 (dd, J=13.4, 5.5 Hz, 1H), 2.28-2.20 (m), 2.19-2.13 (m), 2.11 (s, 3H), 2.06-1.96 (m), 1.83 (s, 3H), 1.68 (s, 3H), 1.27-1.14 (m), 1.00 (s, 3H).

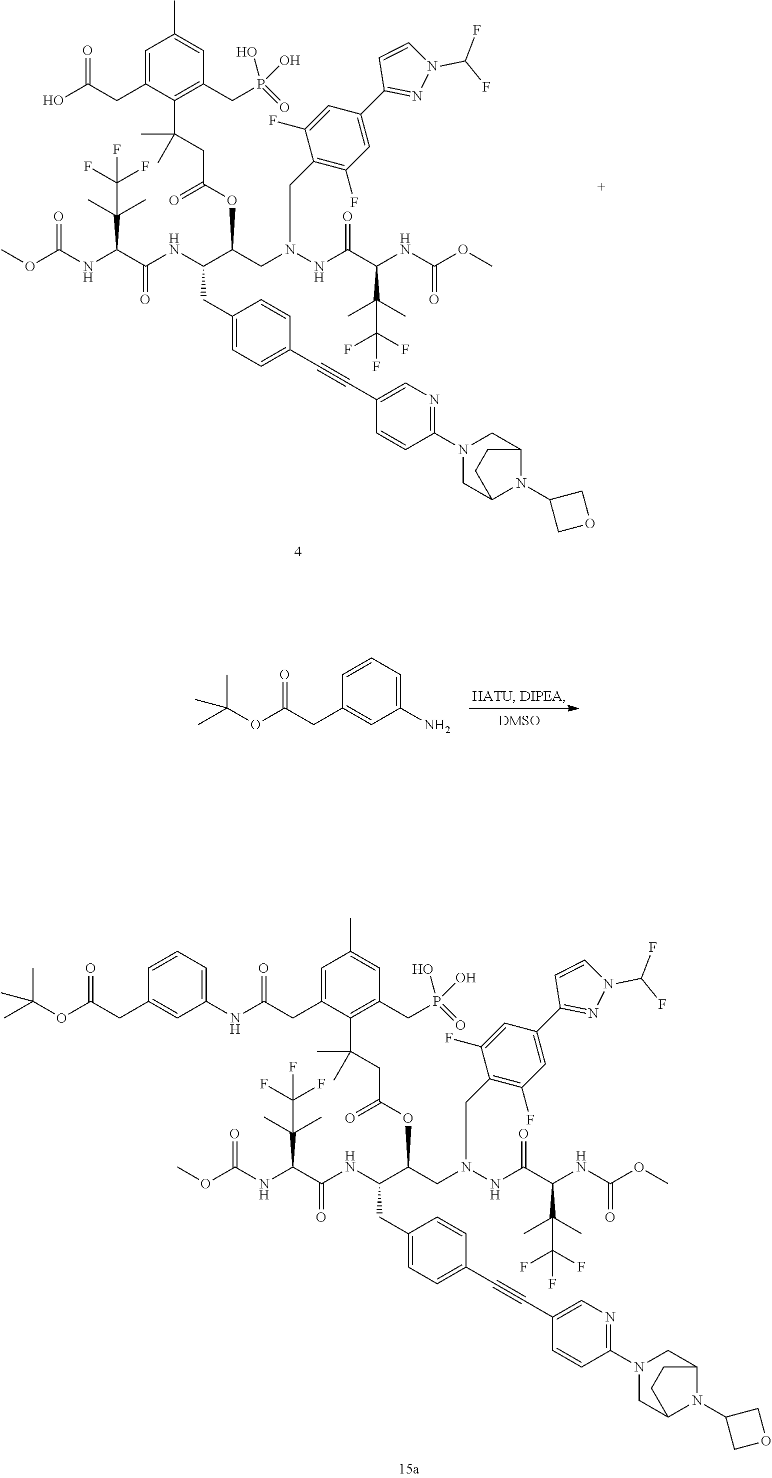 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1CP(=O)(O)O)C(C)(C)C(F)(F)F ; CC(C)(C)OC(=O)Cc1cccc(N)c1 ; COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(CC(=O)OC(C)(C)C)c2)cc(C)cc1CP(=O)(O)O)C(C)(C)C(F)(F)F]

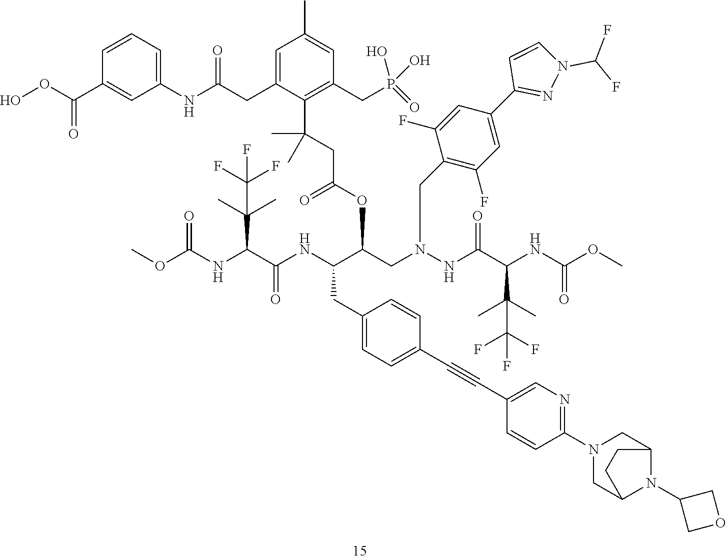 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(C(=O)OO)c2)cc(C)cc1CP(=O)(O)O)C(C)(C)C(F)(F)F]

### Example 15

Compound 15 was prepared according to example 14, substituting tert-butyl 2-(3-aminophenyl)acetate for tert-butyl 3-aminobenzoate. MS (m/z): 1618.1 [M+H]⁺. ¹H NMR (400 MHz, Methanol-d4) δ 8.54 (s, 2H), 8.11 (d, J=2.8 Hz, 1H), 7.72-7.37 (m), 7.28 (t, J=7.2 Hz, 3H), 7.24 (d, J=7.9 Hz, 1H), 7.08 (d, J=7.9 Hz, 2H), 7.04-6.98 (m), 6.94 (d, J=2.8 Hz, 1H), 6.77 (s, 1H), 5.04-4.94 (m), 4.85-4.74 (m), 4.58-4.09 (m), 4.01-3.85 (m), 3.74 (s, 3H), 3.67 (s, 3H), 3.58 (s, 2H), 3.56-3.44 (m), 3.07-2.95 (m), 2.90 (d, J=15.5 Hz, 1H), 2.86-2.75 (m), 2.38-2.19 (m), 2.10 (s, 3H), 2.06-1.94 (m), 1.84 (s, 3H), 1.67 (s, 3H), 1.27-1.12 (m), 1.01 (s, 3H).

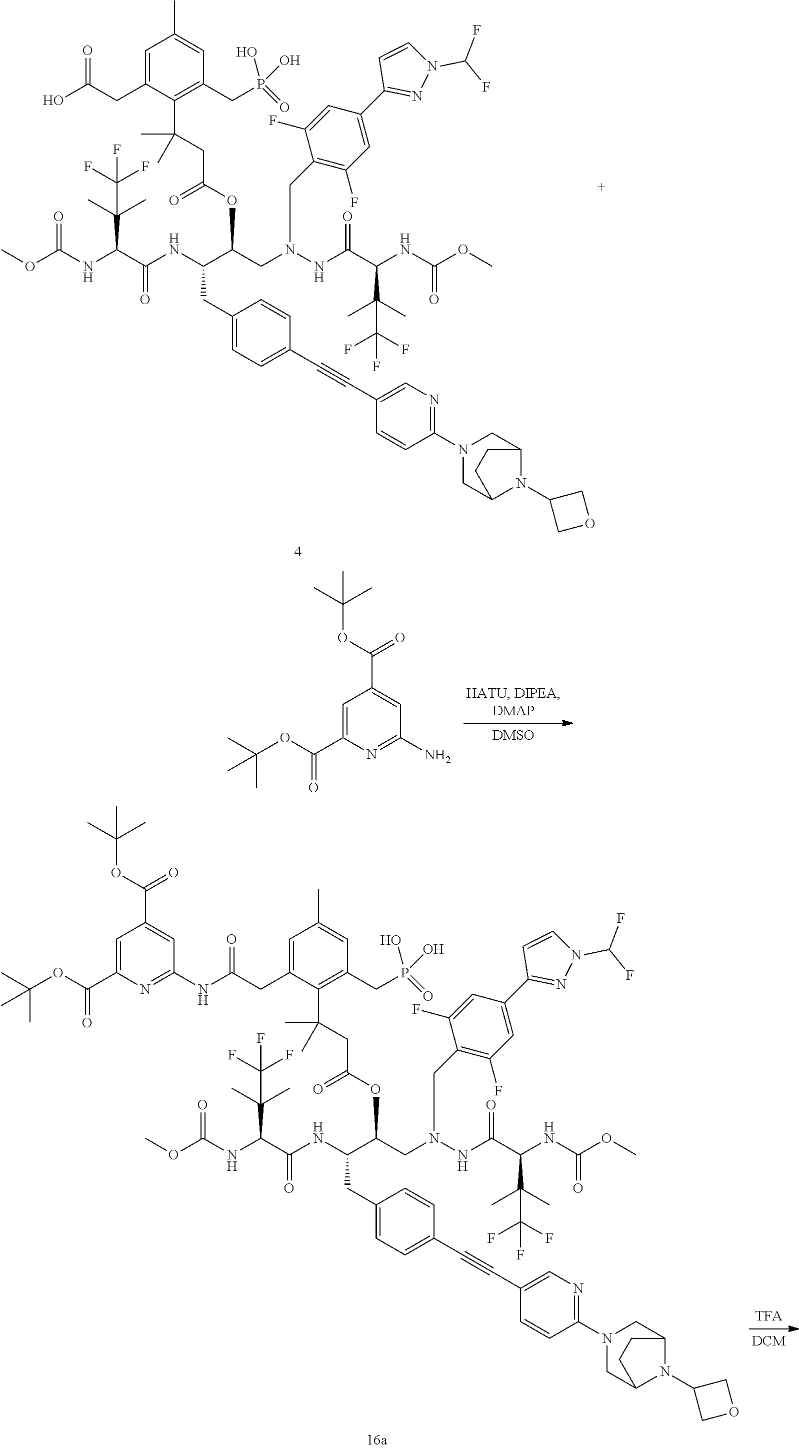

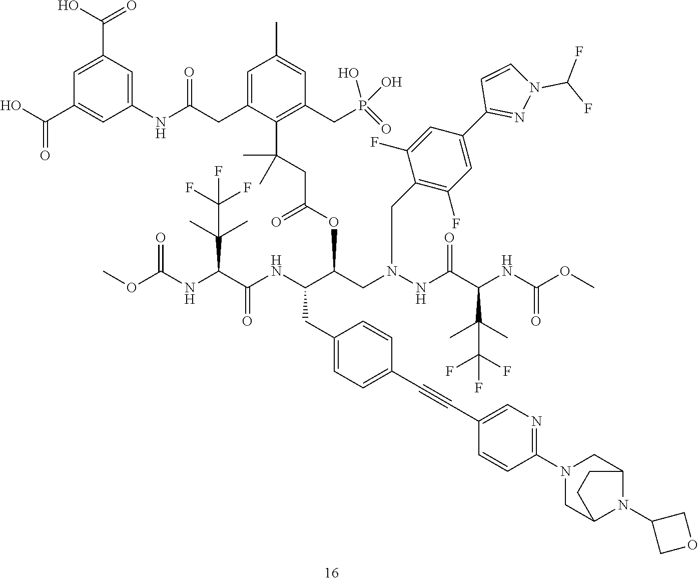 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)O)cc(C(=O)O)c2)cc(C)cc1CP(=O)(O)O)C(C)(C)C(F)(F)F]

### Example 16

Compound 16 was prepared according to example 14, substituting di-tert-butyl 5-aminoisophthalate for tert-butyl 3-aminobenzoate. MS (m/z): 1648.1 [M+H]⁺. ¹H NMR (400 MHz, Methanol-d4) δ 8.53 (s, 2H), 8.47 (s, 2H), 8.35 (s, 1H), 8.10 (d, J=2.7 Hz, 1H), 7.76-7.36 (m), 7.34-7.21 (m), 7.09 (d, J=7.9 Hz, 2H), 6.98-6.87 (m), 6.81 (s, 1H), 5.09-4.94 (m), 4.84-4.70 (m), 4.39-4.25 (m), 4.25-4.11 (m), 4.07 (d, J=17.3 Hz, 1H), 3.91 (d, J=13.2 Hz, 1H), 3.73 (s, 3H), 3.66 (s, 3H), 3.59-3.42 (m), 3.20-2.93 (m), 2.88 (dd, J=13.2, 6.8 Hz, 1H), 2.42-2.30 (m), 2.30-2.15 (m), 2.11 (s, 3H), 2.06-1.93 (m), 1.84 (s, 3H), 1.69 (s, 3H), 1.40-1.28 (m), 1.24-1.10 (m), 0.99 (s, 3H).

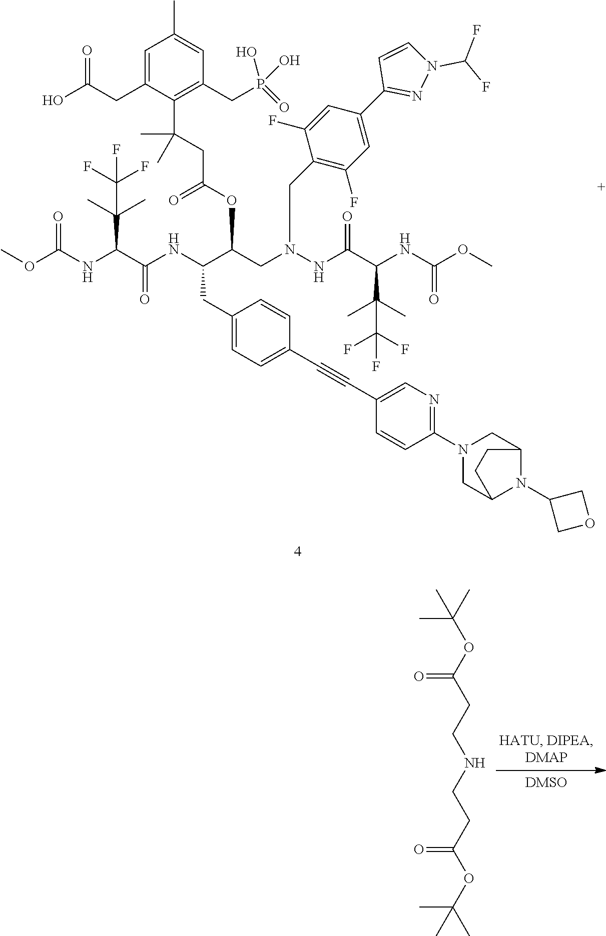 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1CP(=O)(O)O)C(C)(C)C(F)(F)F ; CC(C)(C)OC(=O)CCNCCC(=O)OC(C)(C)C]

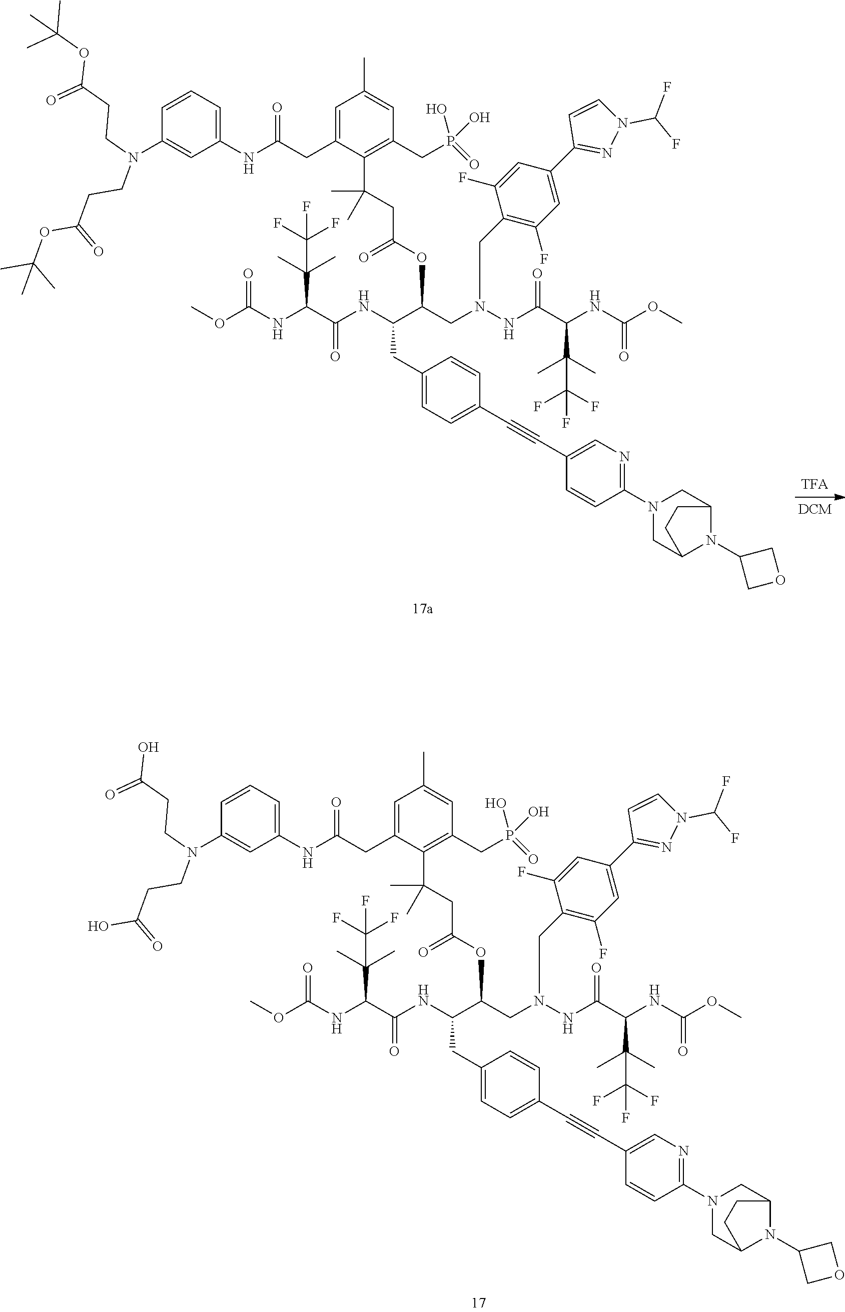

### Example 17

Compound 17 was prepared according to example 14, substituting di-tert-butyl 3,3′-azanediyldipropionate for tert-butyl 3-aminobenzoate. MS (m/z): 1628.3 [M+H]⁺. ¹H NMR (400 MHz, Methanol-d4) δ 8.54 (s, 2H), 8.12 (d, J=2.8 Hz, 1H), 7.74-7.38 (m), 7.35-7.22 (m), 7.12 (d, J=7.9 Hz, 2H), 6.98-6.85 (m), 6.66-6.49 (m), 4.98 (t, J=7.6 Hz, 2H), 4.85-4.71 (m), 4.60-4.27 (m), 4.26-4.08 (m), 4.06-3.87 (m), 3.85-3.72 (m), 3.71-3.58 (m), 3.58-3.47 (m), 3.41 (d, J=15.5 Hz, 1H), 3.06-2.94 (m), 2.87 (d, J=15.5 Hz, 1H), 2.77 (dd, J=13.1, 6.3 Hz, 1H), 2.73-2.60 (m), 2.40-2.16 (m), 2.15-1.93 (m), 1.74 (s, 3H), 1.63 (s, 3H), 1.39-1.28 (m), 1.27-1.09 (m), 1.01 (s, 3H).

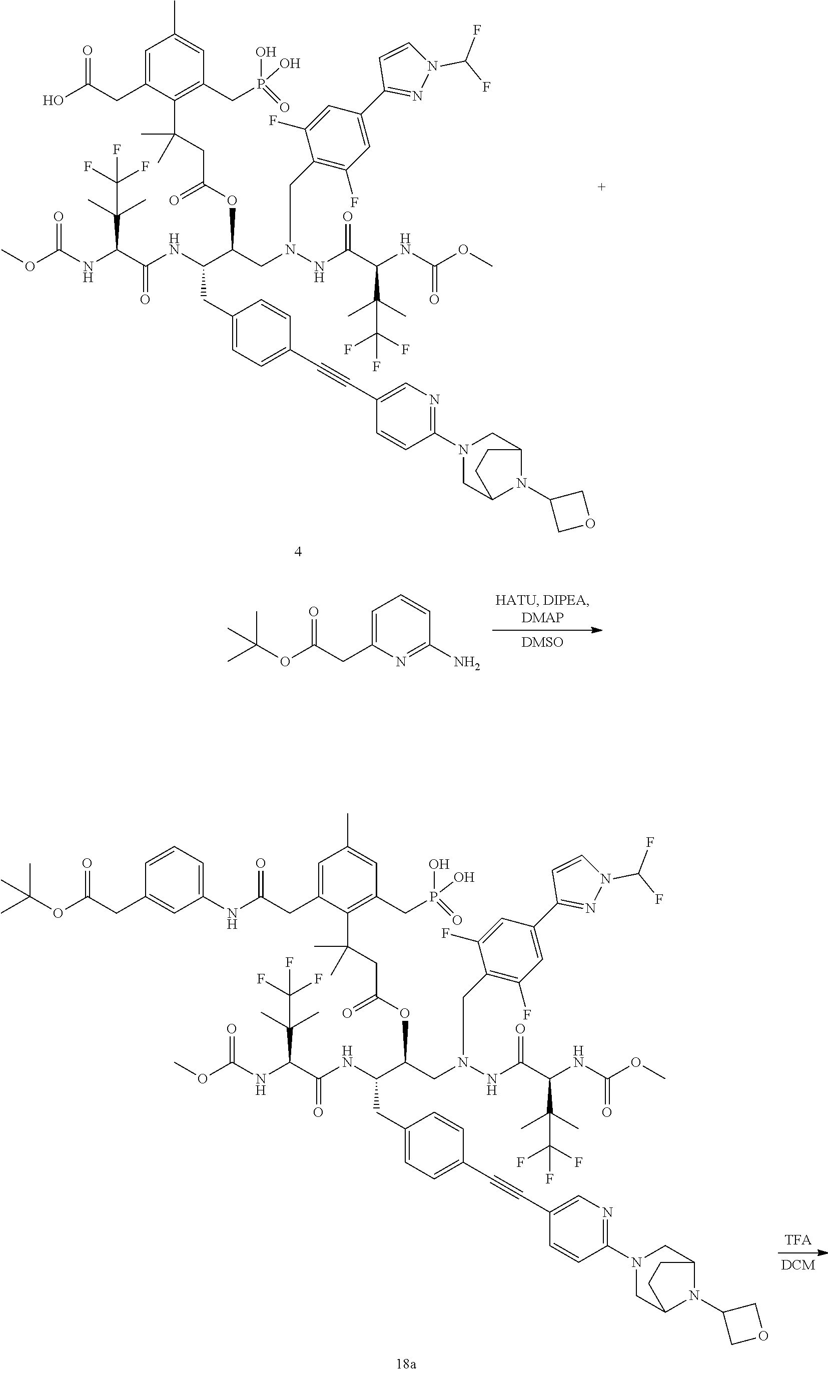

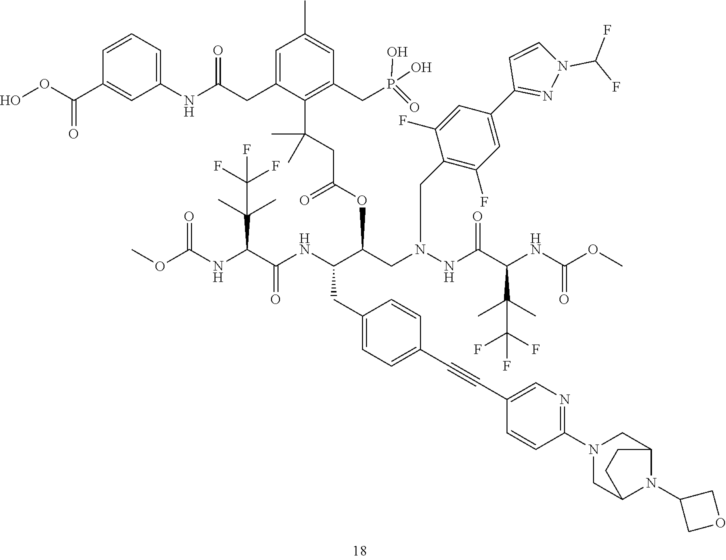 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(C(=O)OO)c2)cc(C)cc1CP(=O)(O)O)C(C)(C)C(F)(F)F]

### Example 18

Compound 18 was prepared according to example 14, substituting tert-butyl 4-aminobenzoate for tert-butyl 3-aminobenzoate. MS (m/z): 1604.2 [M+H]⁺. ¹H NMR (400 MHz, Methanol-d4) δ 8.54 (s, 2H), 8.11 (d, J=2.7 Hz, 1H), 7.96 (d, J=8.8 Hz, 2H), 7.68 (d, J=8.7 Hz, 2H), 7.60-7.37 (m), 7.36-7.21 (m), 7.10 (d, J=7.9 Hz, 2H), 6.98-6.64 (m), 4.98 (t, J=7.6 Hz, 2H), 4.85-4.73 (m), 4.41-4.26 (m), 4.24-4.10 (m), 3.94 (dd, J=31.2, 15.3 Hz, 2H), 3.74 (s, 3H), 3.67 (s, 3H), 3.60-3.39 (m), 3.08-2.93 (m), 2.93-2.76 (m), 2.43-2.28 (m), 2.28-2.18 (m), 2.12 (s, 3H), 2.07-1.94 (m), 1.82 (s, 3H), 1.67 (s, 3H), 1.44-1.27 (m), 1.20 (d, J=7.1 Hz, 9H), 1.01 (s, 3H).

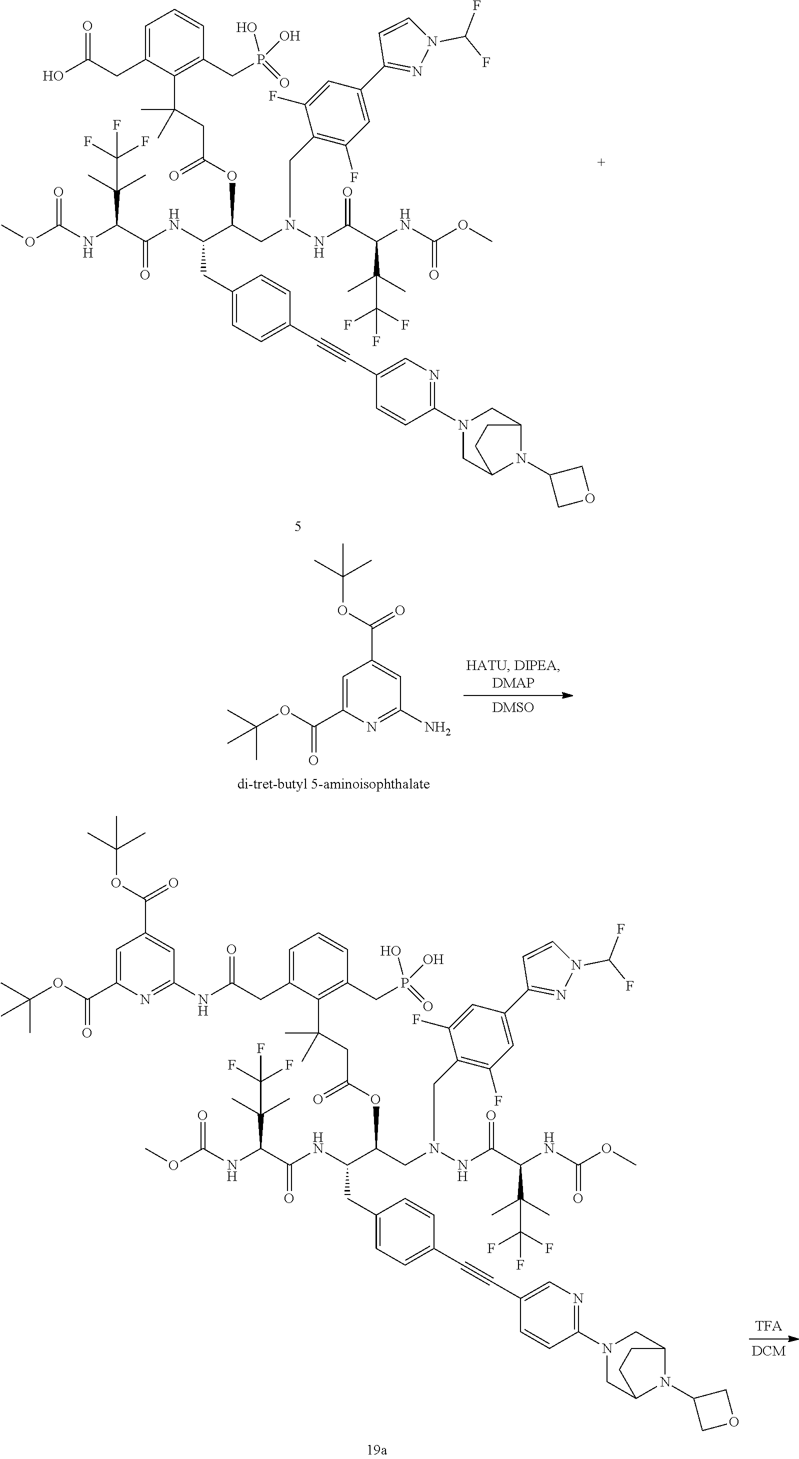

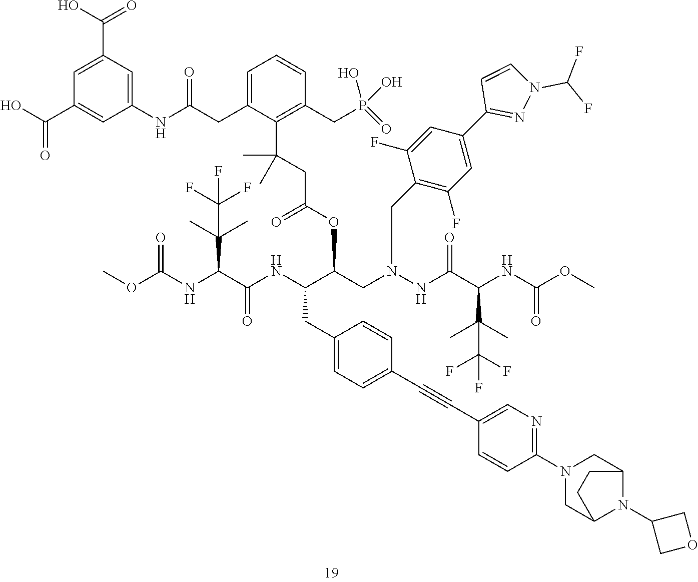 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)O)cc(C(=O)O)c2)cccc1CP(=O)(O)O)C(C)(C)C(F)(F)F]

### Example 19

Synthesis of di-tert-butyl 5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)isophthalate (19a): To a solution of 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetic acid 5 (0.17 mmol) in DMSO (1.0 mL) was added HATU (0.52 mmol), DIPEA (0.87 mmol), di-tert-butyl 5-aminoisophthalate (0.47 mmol), and 4-dimethylaminopyridine (0.2 mmol). The reaction mixture was stirred at rt for 4 hours. The crude reaction mixture was directly purified via reverse-phase HPLC (eluent: water/MeCN containing 0.1% TFA) to provide di-tert-butyl 5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)isophthalate 19a. MS (m/z): 1745.1 [M+H]⁺.

Synthesis of 5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)isophthalic acid (19): To a solution of di-tert-butyl 5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)isophthalate 19a (0.073 mmol) in DCM (3 mL) was added TFA (1.5 mL). The reaction mixture was stirred for 90 mins at rt. The crude reaction mixture was concentrated under vacuum. The crude residue was purified by reverse-phase HPLC (eluent: water/MeCN containing 0.1% TFA) to provide 5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)isophthalic acid 19. MS (m/z): 1634.8 [M+H]⁺. ¹H NMR (400 MHz, Methanol-d4) δ 8.52 (s, 2H), 8.46 (d, J=1.5 Hz, 2H), 8.33 (s, 1H), 8.10 (d, J=2.7 Hz, 1H), 7.76-7.34 (m), 7.27 (d, J=7.8 Hz, 2H), 7.18-6.98 (m), 6.96-6.84 (m), 5.04-4.93 (m), 4.84-4.65 (m), 4.59-4.25 (m), 4.22-4.07 (m), 3.87 (d, J=13.2 Hz, 1H), 3.73 (s, 3H), 3.65 (s, 3H), 3.54-3.42 (m), 3.20-2.94 (m), 2.90-2.75 (m), 2.52-2.15 (m), 2.09-1.91 (m), 1.84 (s, 3H), 1.72 (s, 3H), 1.43-1.27 (m), 1.27-1.09 (m), 0.96 (s, 3H).

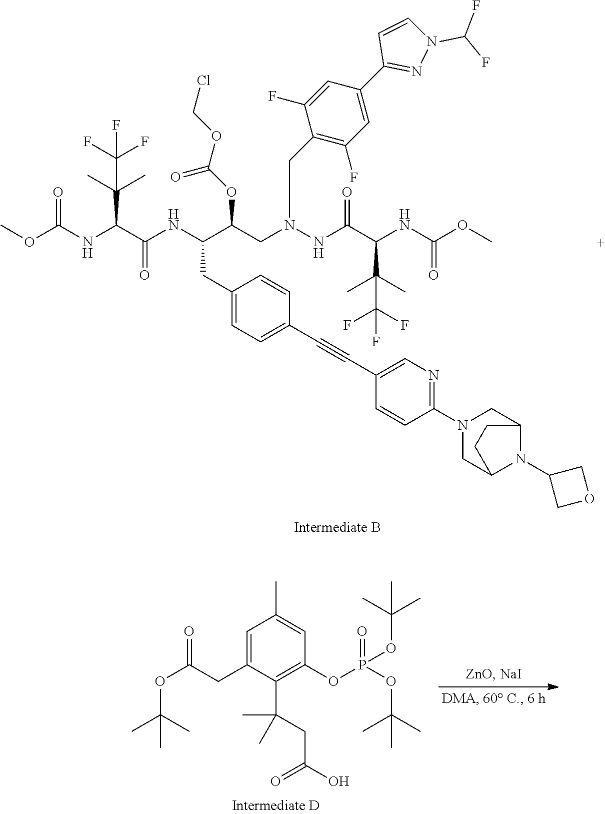 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCCl)C(C)(C)C(F)(F)F ; Cc1cc(CC(=O)OC(C)(C)C)c(C(C)(C)CC(=O)O)c(OP(=O)(OC(C)(C)C)OC(C)(C)C)c1]

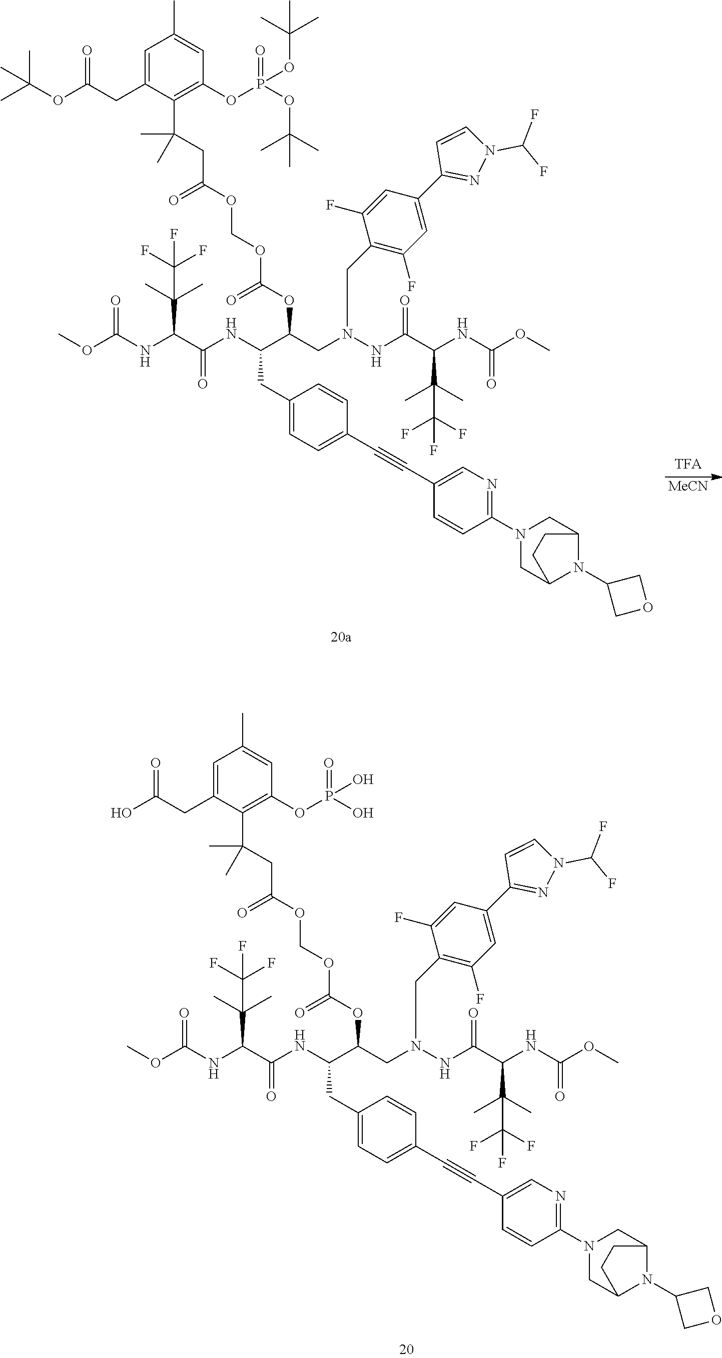 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)OC(C)(C)C)cc(C)cc1OP(=O)(OC(C)(C)C)OC(C)(C)C)C(C)(C)C(F)(F)F ; "MeCN" ; COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F]

### Example 20

Synthesis of (5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2-(2-(tert-butoxy)-2-oxoethyl)-6-((di-tert-butoxyphosphoryl)oxy)-4-methylphenyl)-3-methylbutanoate (20a): To a solution of Intermediate B (0.060 mmol) and 3-(2-(2-(tert-butoxy)-2-oxoethyl)-6-((di-tert-butoxyphosphoryl)oxy)-4-methylphenyl)-3-methylbutanoic acid (Intermediate D) (0.15 mmol) in DMA (1.0 mL) was added zinc oxide (0.30 mmol) and sodium iodide (0.24 mmol). The RM was stirred at 60° C. for 6 hours, then filtered through a pad of celite, and directly purified by RP-HPLC (eluent: water/MeCN 0.1% TFA) to provide (5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2-(2-(tert-butoxy)-2-oxoethyl)-6-((di-tert-butoxyphosphoryl)oxy)-4-methylphenyl)-3-methylbutanoate (20a). MS (m/z): 1726.9 [M+H]⁺.

Synthesis of 2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetic acid (20): To a solution of 20a (0.030 mmol) in DCM (0.5 mL) was added trifluoroacetic acid (6.0 mmol). The RM was stirred at RT for 15 minutes, then concentrated in vacuo and purified by RP-HPLC (eluent: water/MeCN 0.1% TFA) to provide 2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetic acid (20). MS (m/z): 1559.2 [M+H]⁺. ¹H NMR (400 MHz, Methanol-d4) δ 8.53 (s, 2H), 8.10 (d, J=2.7 Hz, 1H), 7.72-7.37 (m), 7.37-7.32 (m), 7.27-7.17 (m), 7.00 (d, J=10.1 Hz, 1H), 6.93 (d, J=2.7 Hz, 1H), 6.71 (s, 1H), 5.71 (d, J=5.9 Hz, 1H), 5.61 (d, J=5.9 Hz, 1H), 5.02-4.92 (m), 4.84-4.74 (m), 4.67-4.55 (m), 4.49-4.42 (m), 4.34-4.23 (m), 4.14 (s, 2H), 4.06 (d, J=13.3 Hz, 1H), 3.94 (s, 2H), 3.72-3.64 (m), 3.49 (s, 1H), 3.46 (s, 1H), 3.24 (dd, J=13.4, 6.0 Hz, 1H), 3.14 (s, 2H), 3.06 (s, 3H), 2.99-2.84 (m), 2.78-2.68 (m), 2.25-2.17 (m), 2.08 (s, 3H), 2.04-1.94 (m), 1.62 (s, 6H), 1.20 (s, 3H), 1.17 (s, 3H), 1.13 (s, 3H), 1.05 (s, 3H).

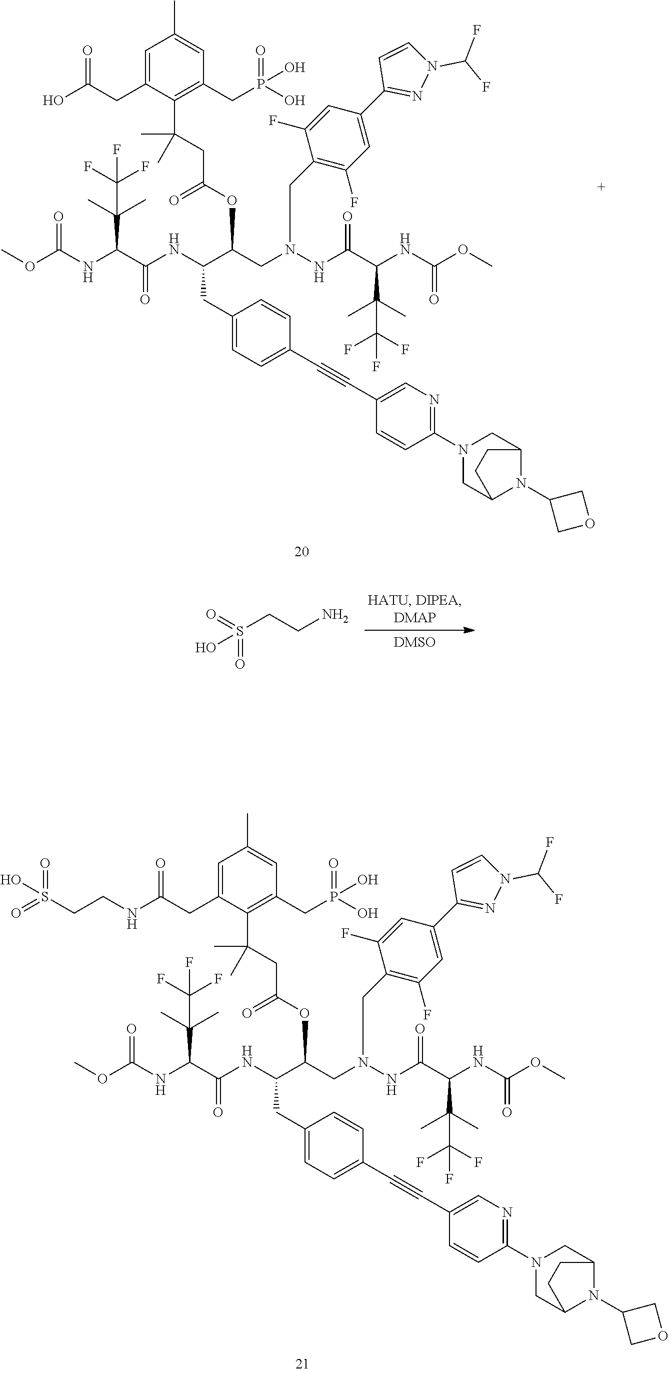 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1CP(=O)(O)O)C(C)(C)C(F)(F)F ; NCCS(=O)(=O)O ; COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCS(=O)(=O)O)cc(C)cc1CP(=O)(O)O)C(C)(C)C(F)(F)F]

### Example 21

To a solution of 20 (0.032 mmol), taurine (0.13 mmol), HATU (0.13 mmol) and DMAP (0.0063 mmol) in DMSO (1.0 mL) was added DIPEA (0.14 mmol). The RM was stirred at RT for 20 minutes then purified by RP-HPLC (eluent: water/MeCN 0.1% TFA) to provide 2-(2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetamido)ethane-1-sulfonic acid (21). MS (m/z): 1666.1 [M+H]⁺. ¹H NMR (400 MHz, Methanol-d4) δ 8.53 (s, 2H), 8.10 (d, J=2.6 Hz, 1H), 7.71-7.37 (m), 7.37-7.31 (m), 7.28-7.19 (m), 6.93 (d, J=2.7 Hz, 1H), 6.74 (s, 1H), 5.75 (d, J=5.9 Hz, 1H), 5.59 (d, J=5.9 Hz, 1H), 5.06-4.93 (m), 4.87-4.74 (m), 4.68-4.56 (m), 4.56-4.39 (m), 4.37-4.22 (m), 4.14 (s, 2H), 4.05 (d, J=13.2 Hz, 1H), 3.93-3.75 (m), 3.72-3.66 (m), 3.65-3.55 (m), 3.50 (s, 1H), 3.46 (s, 1H), 3.29-3.19 (m), 3.16-3.11 (m), 3.00-2.91 (m), 2.88 (dd, J=14.1, 6.1 Hz, 1H), 2.81-2.72 (m), 2.28-2.16 (m), 2.03-1.95 (m), 1.63-1.54 (m), 1.20 (s, 3H), 1.17 (s, 3H), 1.13 (s, 3H), 1.03 (s, 3H).

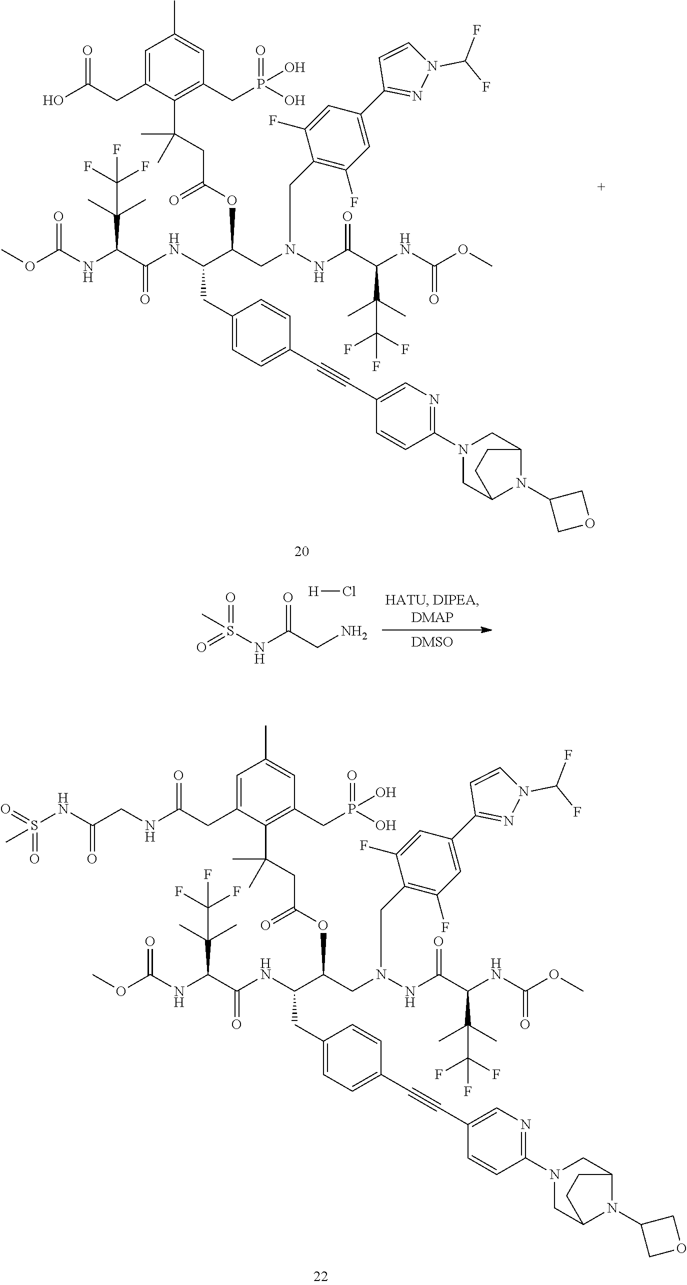

### Example 22

(5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-methyl-3-(4-methyl-2-(2-((2-(methylsulfonamido)-2-oxoethyl)amino)-2-oxoethyl)-6-(phosphonooxy)phenyl)butanoate (22) was synthesized using the method described in Example 22, substituting Intermediate Q for taurine. MS (m/z): 1692.9 [M+H]⁺. ¹H NMR (400 MHz, Methanol-d4) δ 8.53 (s, 2H), 8.10 (d, J=2.7 Hz, 1H), 7.71-7.38 (m), 7.37-7.31 (m), 7.29-7.18 (m), 7.01 (d, J=10.1 Hz, 1H), 6.93 (d, J=2.8 Hz, 1H), 6.84-6.73 (m), 5.73 (d, J=5.9 Hz, 1H), 5.60 (d, J=5.9 Hz, 1H), 5.02-4.92 (m), 4.85-4.70 (m), 4.64-4.55 (m), 4.54-4.46 (m), 4.35-4.31 (m), 4.28 (d, J=13.3 Hz, 1H), 4.14 (s, 2H), 4.09-3.86 (m), 3.70 (s, 3H), 3.67 (s, 3H), 3.52-3.48 (m), 3.46 (s, 1H), 3.24-3.17 (m), 3.11-3.04 (m), 3.00-2.84 (m), 2.79-2.67 (m), 2.27-2.15 (m), 2.06-1.94 (m), 1.71-1.52 (m), 1.21 (s, 3H), 1.17 (s, 3H), 1.13 (s, 3H), 1.05 (s, 3H).

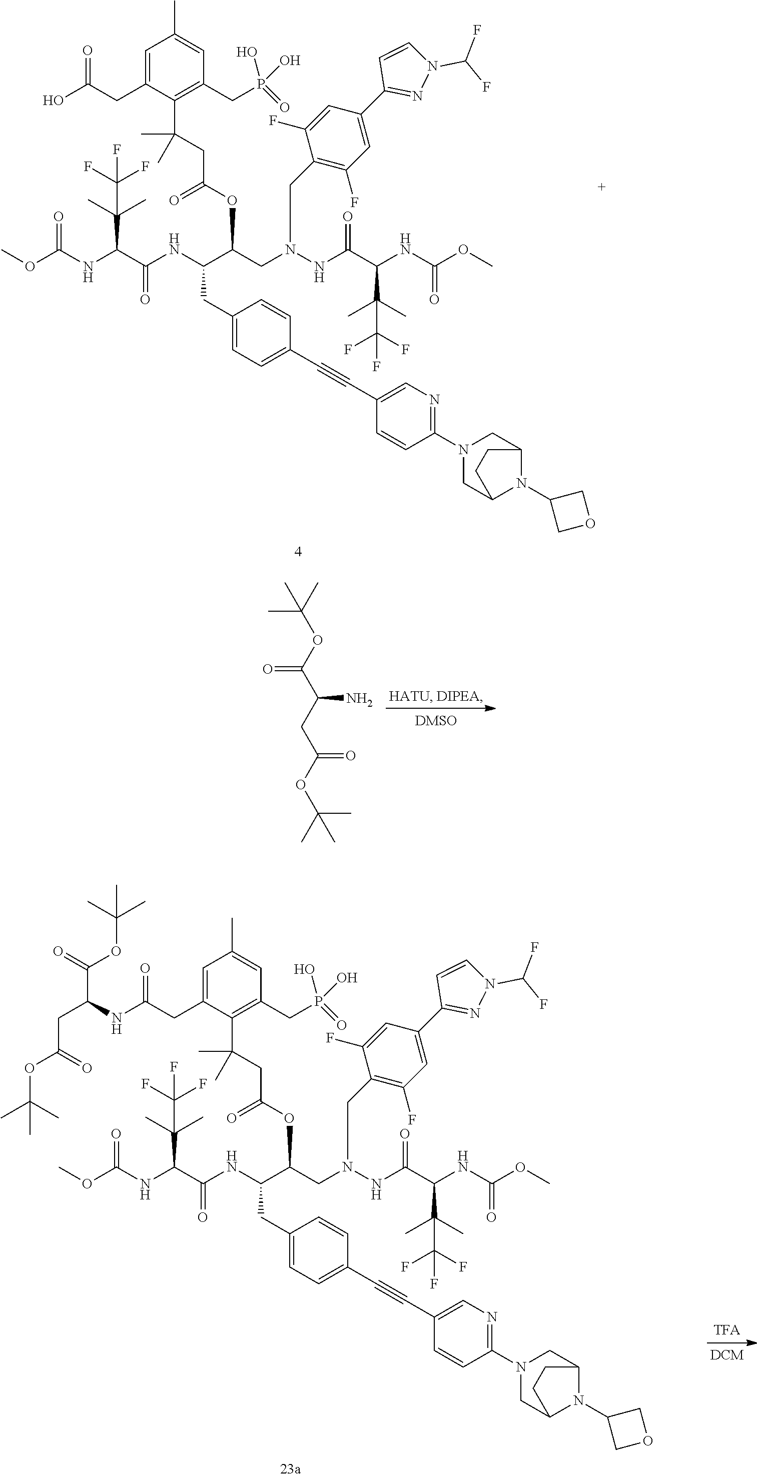

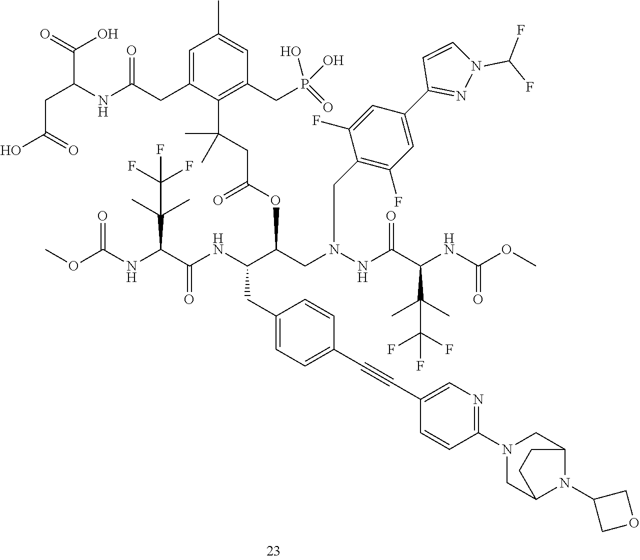 [SMILES: COC(=O)N[C@H](C(=O)N[C@@H](Cc1ccc(C#Cc2ccc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC(CC(=O)O)C(=O)O)cc(C)cc1CP(=O)(O)O)C(C)(C)C(F)(F)F]

### Example 23

Synthesis of di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-aspartate (23a): To a solution of 4 (1.0 mmol), di-tert-butyl L-aspartate (2.0 mmol), and HATU (3.0 mmol) in DCM (10.0 mL) was added DIPEA (6.01 mmol). The RM was stirred at RT for 1 h, then concentrated and purified by RP-MPLC (eluent: water/MeCN 0.1% TFA) to provide di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-aspartate (23a). MS (m/z): 1712.1 [M+H]⁺.

Synthesis of di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-aspartate (23): To a solution of 23a (1.0 mmol) in DCM (2 mL) was added trifluoroacetic acid (25.0 mmol). The RM was stirred at RT for 20 minutes, then concentrated and purified by RP-MPLC (eluent: water/MeCN 0.1% TFA) to provide (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-aspartate (23). MS (m/z): 1600.0 [M+H]⁺. ¹H NMR (400 MHz, Methanol-d4) δ 8.52 (s, 2H), 8.10 (d, J=2.7 Hz, 1H), 7.73-7.35 (m), 7.35-7.20 (m), 7.14-7.02 (m), 6.94 (d, J=2.8 Hz, 1H), 6.90 (d, J=10.1 Hz, 1H), 6.70 (s, 1H), 6.69-6.61 (m), 5.03-4.92 (m), 4.87-4.73 (m), 4.53 (s, 1H), 4.39-4.25 (m), 4.21 (d, J=6.2 Hz, 1H), 4.18-4.07 (m), 4.01-3.87 (m), 3.82-3.63 (m), 3.62-3.39 (m), 3.01-2.90 (m), 2.88-2.82 (m), 2.83-2.70 (m), 2.37-2.14 (m), 2.13-1.90 (m), 1.77 (s, 3H), 1.63 (s, 3H), 1.27-0.93 (m).

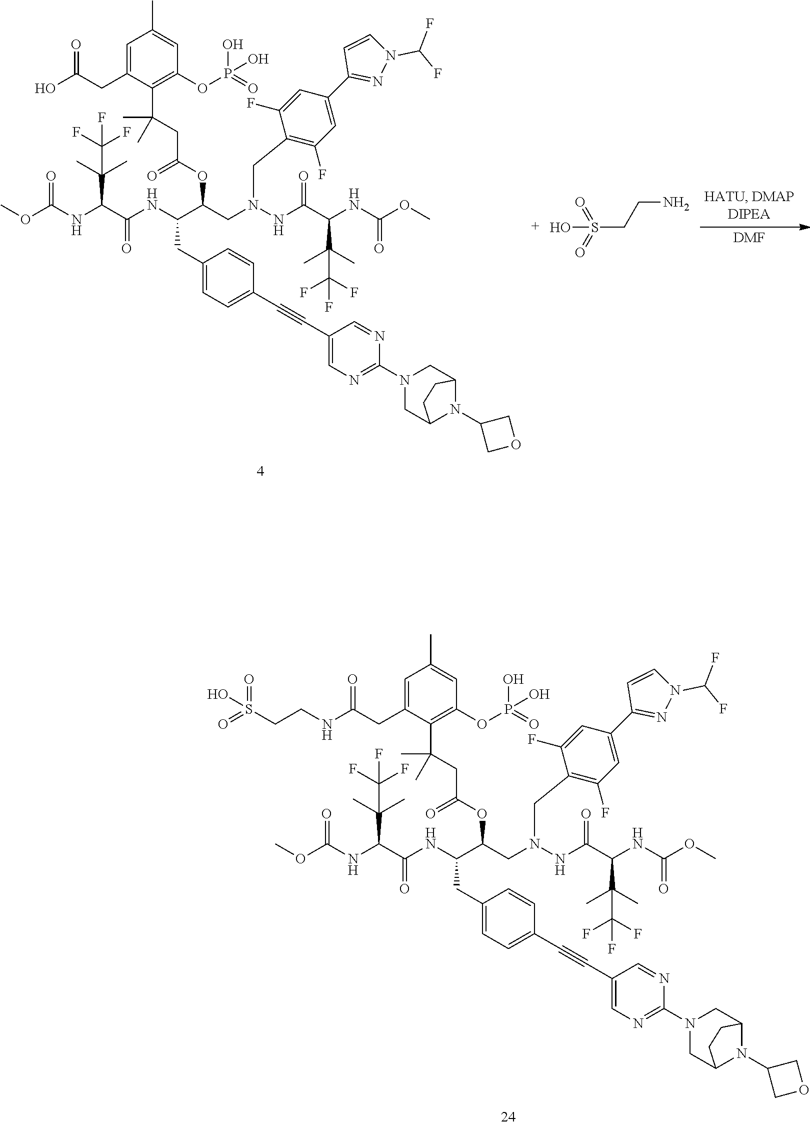

### Example 24

To a solution of 4 (0.025 mmol), taurine (0.078 mmol), HATU (0.078 mmol) and DMAP (0.0050 mmol) in DMF (1.0 mL) was added DIPEA (0.088 mmol). The RM was stirred at 45° C. for 1 h then purified by RP-HPLC (eluent: water/MeCN 0.1% TFA) to provide 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)ethane-1-sulfonic acid (24). MS (m/z): 1592.1 [M+H]⁺. ¹H NMR (400 MHz, Methanol-d4) δ 8.52 (s, 2H), 8.10 (d, J=2.7 Hz, 1H), 7.74-7.37 (m), 7.33-7.26 (m), 7.24 (s, 1H), 7.14-7.03 (m), 6.94 (d, J=2.8 Hz, 1H), 6.71 (s, 1H), 5.01-4.93 (m), 4.87-4.71 (m), 4.47 (s, 1H), 4.36 (s, 1H), 4.31-4.25 (m), 4.24-4.08 (m), 3.92 (d, J=13.3 Hz, 1H), 3.72 (s, 3H), 3.69-3.59 (m), 3.52-3.47 (m), 3.45 (s, 1H), 3.43-3.35 (m), 3.03-2.90 (m), 2.89-2.66 (m), 2.35-2.16 (m), 2.09 (s, 4H), 2.04-1.96 (m), 1.75 (s, 3H), 1.62 (s, 3H), 1.25-0.89 (m).

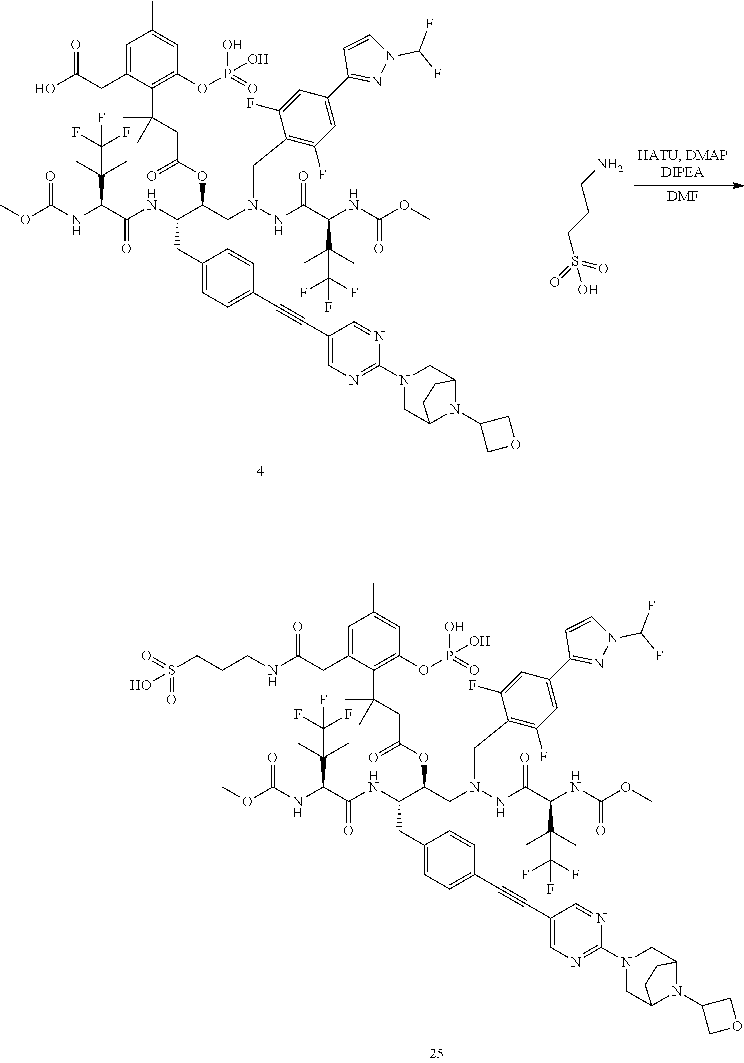

### Example 25

3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)propane-1-sulfonic acid (25) was synthesized using the method described in Example 24, substituting 3-aminopropane-1-sulfonic acid for taurine. MS (m/z): 1606.1 [M+H]⁺. ¹H NMR (400 MHz, Methanol-d4) δ 8.52 (s, 2H), 8.10 (d, J=2.7 Hz, 1H), 7.71-7.36 (m), 7.32-7.25 (m), 7.22 (s, 1H), 7.14-7.03 (m), 6.95 (d, J=2.8 Hz, 1H), 6.71 (s, 1H), 5.01-4.92 (m), 4.84-4.69 (m), 4.47 (s, 1H), 4.41-4.33 (m), 4.30-4.24 (m), 4.21-4.06 (m), 3.92 (d, J=13.4 Hz, 1H), 3.76-3.59 (m), 3.49 (s, 1H), 3.46 (s, 1H), 3.41-3.32 (m), 2.97 (dd, J=13.4, 6.5 Hz, 1H), 2.90-2.79 (m), 2.79-2.71 (m), 2.30 (dd, J=13.6, 6.0 Hz, 1H), 2.25-2.16 (m), 2.09 (s, 3H), 2.04-1.92 (m), 1.75 (s, 3H), 1.63 (s, 3H), 1.22-0.88 (m).

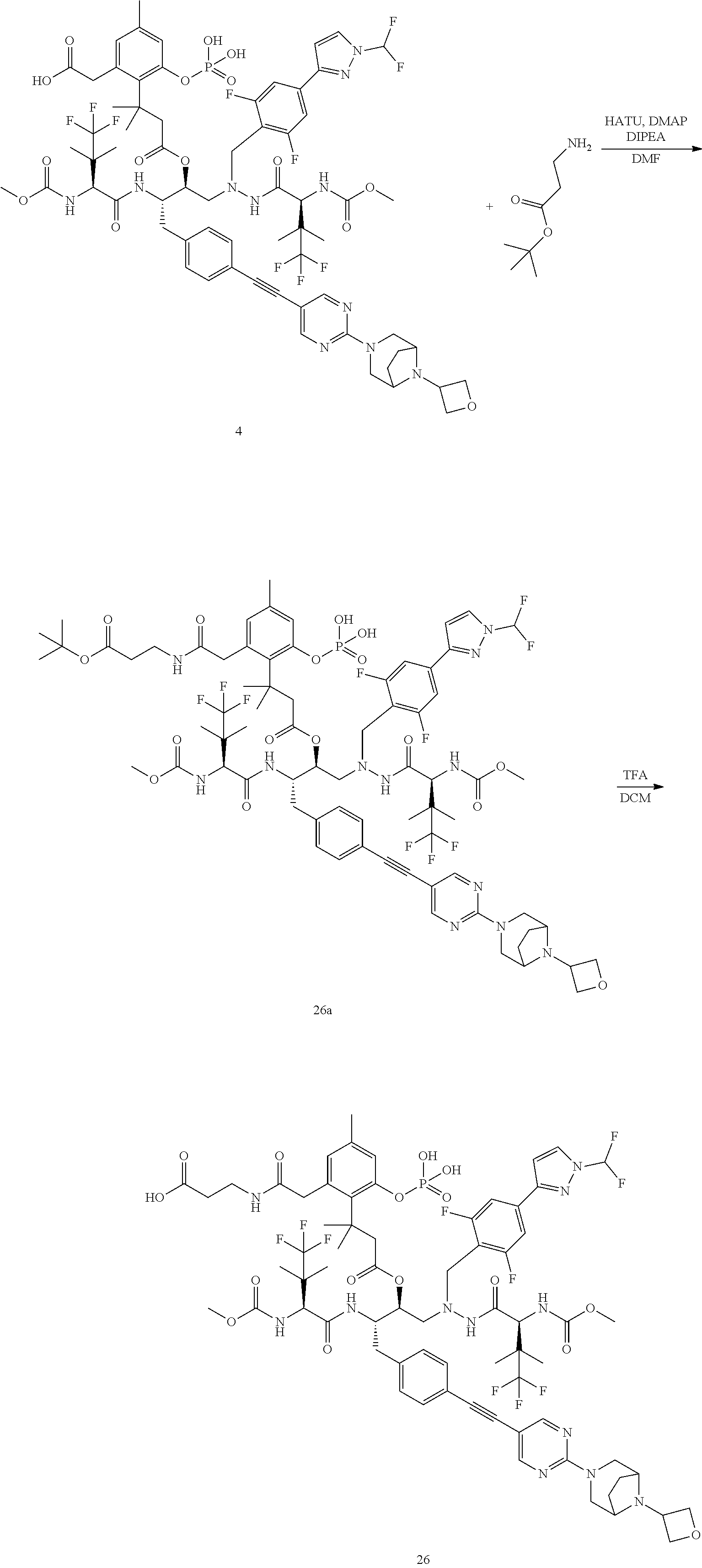

### Example 26

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(tert-butoxy)-3-oxopropyl)amino)-2-oxoethyl)-4-methyl-6-(phosphonooxy)phenyl)-3-methylbutanoate (26a): To a solution of 4 (0.031 mmol), tert-butyl 3-aminopropanoate (0.13 mmol), DMAP (0.0063 mmol), and HATU (0.13 mmol) in DMF (1.0 mL) was added DIPEA (0.19 mmol). The RM was stirred at RT for 30 min then purified by RP-HPLC (eluent: water/MeCN 0.1% TFA) to provide (5S,10S,11S,14S)-8-(4-(1-(difluoromethyl)-1H-pyrazol-3-yl)-2,6-difluorobenzyl)-11-(4-((2-(8-(oxetan-3-yl)-3, 8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(tert-butoxy)-3-oxopropyl)amino)-2-oxoethyl)-4-methyl-6-(phosphonooxy)phenyl)-3-methylbutanoate (26a). MS (m/z): 1612.3 [M+H]⁺.

Synthesis of 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)propanoic acid (26): Compound 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)propanoic acid (26) was synthesized using the method described in Step 2 of Example P-GJB4 substituting 26a for 23a. MS (m/z): 1555.6 [M+H]⁺. ¹H NMR (400 MHz, Methanol-d4) δ 8.55 (s, 2H), 8.12 (d, J=2.7 Hz, 1H), 7.74-7.38 (m), 7.35-7.24 (m), 7.05-7.20 (m), 6.96 (d, J=2.8 Hz, 1H), 6.92 (d, J=10.1 Hz, 1H), 6.79-6.59 (m), 5.02-4.94 (m), 4.86-4.68 (m), 4.42-4.33 (m), 4.33-4.26 (m), 4.25-4.05 (m), 4.00-3.90 (m), 3.80-3.70 (m), 3.70-3.66 (m), 3.57-3.41 (m), 3.35 (s, 1H), 2.99 (dd, J=12.0, 5.3 Hz, 1H), 2.89-2.82 (m), 2.77 (ddd, J=13.1, 5.9, 2.0 Hz, 1H), 2.57-2.48 (m), 2.33 (dd, J=13.5, 5.7 Hz, 1H), 2.24 (d, J=11.2 Hz, 2H), 2.20-2.07 (m), 2.02 (d, J=9.3 Hz, 2H), 1.79-1.72 (m), 1.67-1.55 (m), 1.25-0.91 (m).

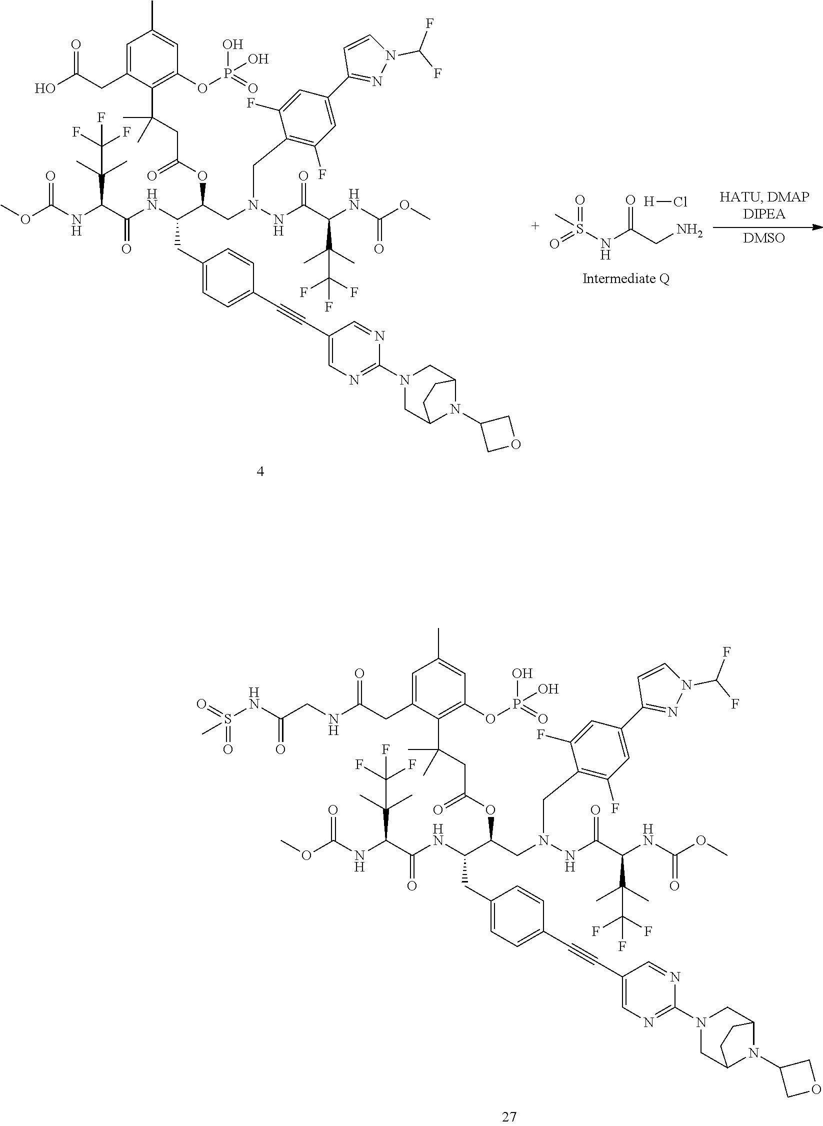

### Example 27

To a solution of 4 (0.029 mmol), Intermediate Q (0.12 mmol), HATU (0.12 mmol) and DMAP (0.0058 mmol) in DMSO (1.0 mL) was added DIPEA (0.23 mmol). The RM was stirred at RT for 15 min then purified by RP-HPLC (eluent: water/MeCN 0.1% TFA) to provide (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-((2-(methylsulfonamido)-2-oxoethyl)amino)-2-oxoethyl)-6-(phosphonooxy)phenyl)butanoate (27). MS (m/z): 1618.5 [M+H]⁺. ¹H NMR (400 MHz, Methanol-d4) δ 8.52 (s, 2H), 8.10 (d, J=2.8 Hz, 1H), 7.72-7.37 (m), 7.33-7.23 (m), 7.14-7.05 (m), 6.94 (d, J=2.8 Hz, 1H), 6.91 (d, J=10.5 Hz, 1H), 6.75 (d, J=1.9 Hz, 1H), 5.03-4.92 (m), 4.86-4.68 (m), 4.52 (s, 1H), 4.44-4.32 (m), 4.32-4.26 (m), 4.23-4.11 (m), 4.07 (d, J=17.4 Hz, 1H), 3.98-3.85 (m), 3.81-3.74 (m), 3.72 (s, 3H), 3.67 (s, 3H), 3.55-3.44 (m), 3.23 (s, 3H), 2.98-2.86 (m), 2.71-2.61 (m), 2.35 (dd, J=13.5, 5.7 Hz, 1H), 2.26-2.19 (m), 2.18-2.04 (m), 2.03-1.95 (m), 1.76 (s, 3H), 1.65 (s, 3H), 1.23-0.90 (m).

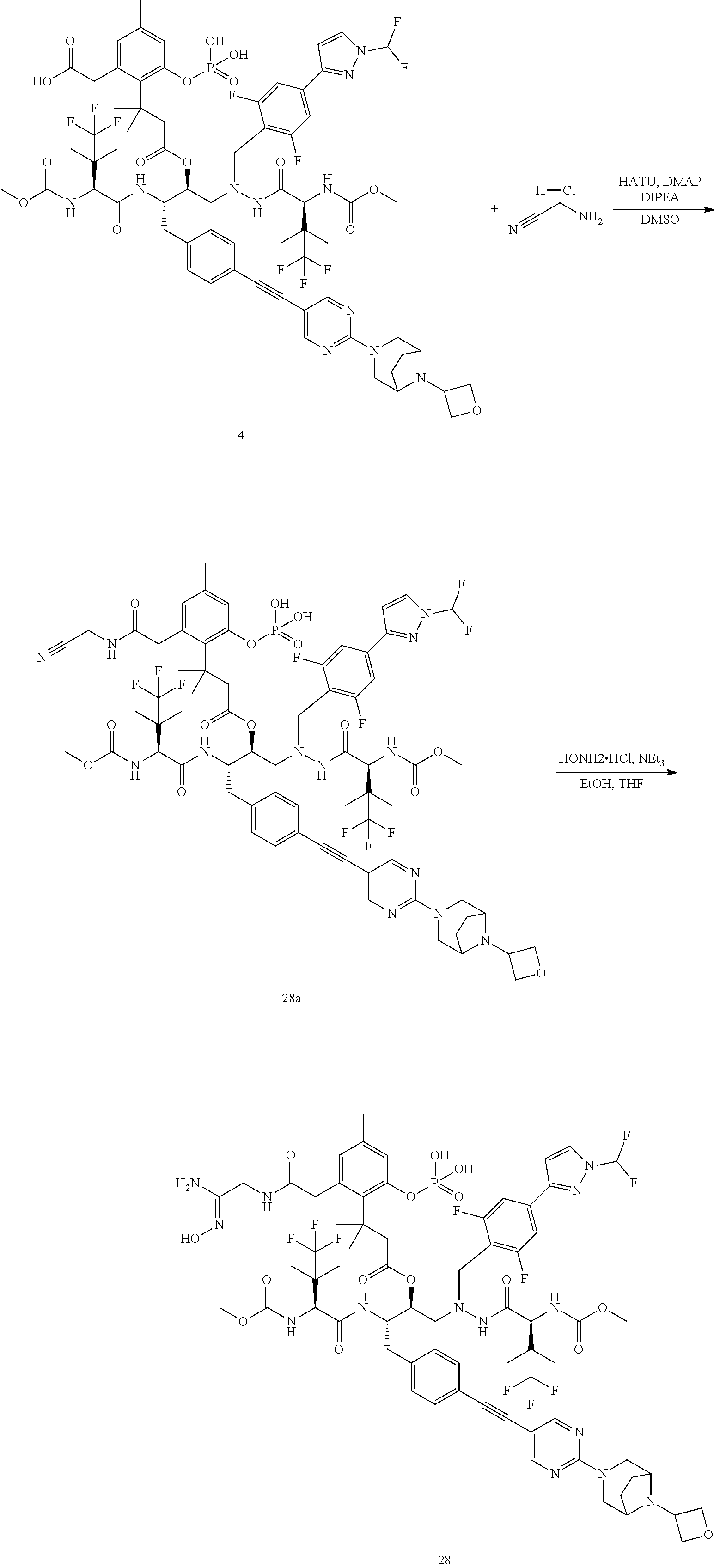

### Example 28

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((cyanomethyl)amino)-2-oxoethyl)-4-methyl-6-(phosphonooxy)phenyl)-3-methylbutanoate (28a): (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((cyanomethyl)amino)-2-oxoethyl)-4-methyl-6-(phosphonooxy)phenyl)-3-methylbutanoate (28a) was synthesized using the method described in Example 27 substituting 2-aminoacetonitrile hydrochloride for Intermediate Q. MS (m/z): 1522.7 [M+H]⁺.

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-(((Z)-2-amino-2-(hydroxyimino)ethyl)amino)-2-oxoethyl)-4-methyl-6-(phosphonooxy)phenyl)-3-methylbutanoate (28): To a solution of P-GJB9-A (0.032 mmol) and hydroxylamine hydrochloride (0.053 mmol) in ethanol (2.0 mL) and THE (0.5 mL) was added triethylamine (0.10 mmol). The RM was stirred at 60° C. for 4 h then concentrated in vacuo and purified by RP-HPLC (eluent: water/MeCN 0.1% TFA) to provide (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-(((Z)-2-amino-2-(hydroxyimino)ethyl)amino)-2-oxoethyl)-4-methyl-6-(phosphonooxy)phenyl)-3-methylbutanoate (28). MS (m/z): 1556.3 [M+H]⁺. ¹H NMR (400 MHz, Methanol-d4) δ 8.54 (s, 2H), 8.11 (d, J=2.7 Hz, 1H), 7.74-7.37 (m), 7.34-7.24 (m), 7.14-7.05 (m), 6.98-6.86 (m), 6.70-6.61 (m), 5.03-4.91 (m), 4.84-4.68 (m), 4.61-4.44 (m), 4.41-4.32 (m), 4.28 (dd, J=10.1, 2.8 Hz, 1H), 4.25-4.07 (m), 3.92 (d, J=13.2 Hz, 1H), 3.79 (dd, J=17.7, 5.0 Hz, 1H), 3.72 (s, 3H), 3.66 (s, 3H), 3.58-3.44 (m), 3.04-2.90 (m), 2.83 (d, J=15.4 Hz, 1H), 2.78-2.67 (m), 2.42-2.28 (m), 2.30-2.17 (m), 2.19-2.07 (m), 2.07-1.94 (m), 1.76-1.66 (m), 1.66-1.53 (m), 1.29-0.87 (m).

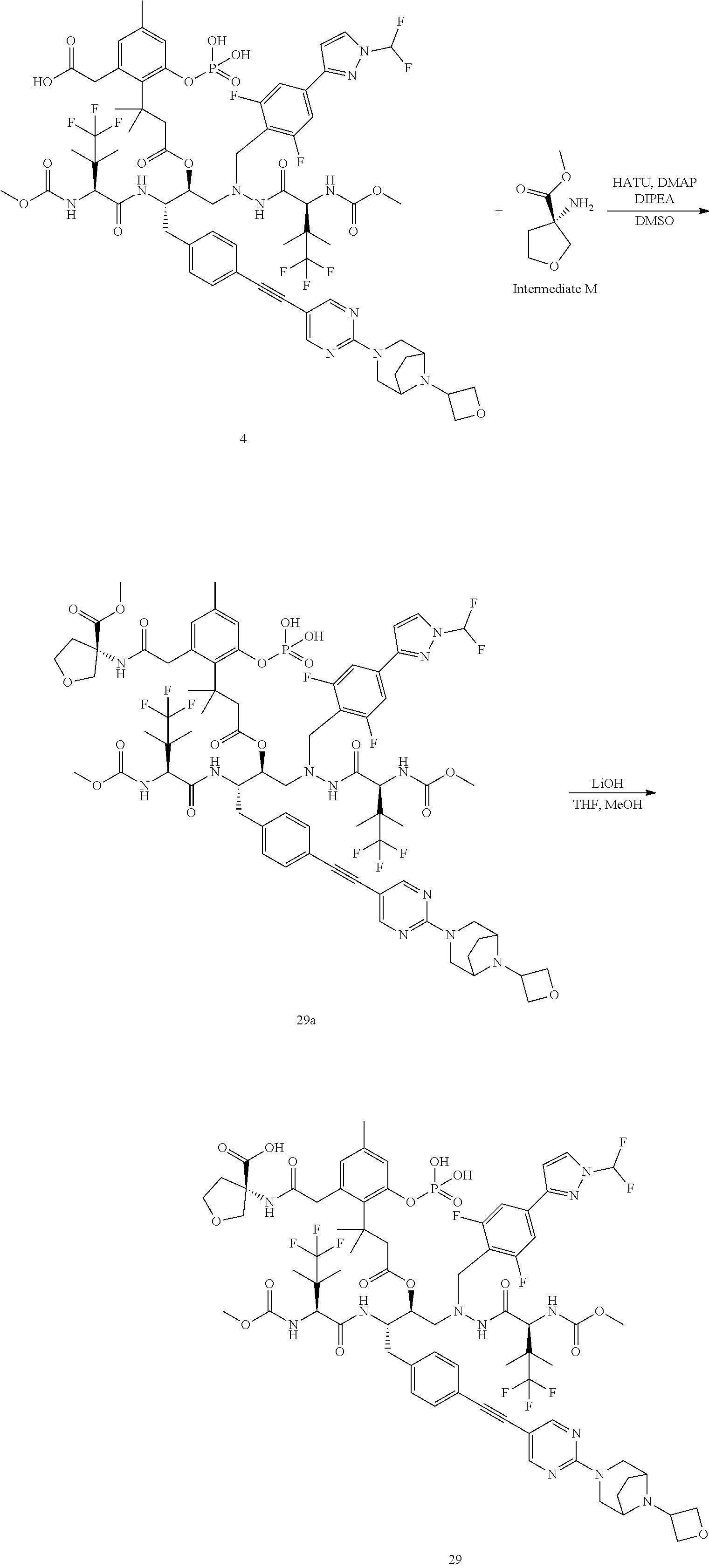

### Example 29

Synthesis of methyl (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylate (29a): To a solution of 4 (0.031 mmol), Intermediate M (0.066 mmol), and DMAP (0.0031 mmol) in DMSO (1.0 mL) was added DIPEA (0.21 mmol) followed by HATU (0.034 mmol). The RM was stirred at RT for 10 min then purified by RP-HPLC (eluent: water/MeCN 0.1% TFA) to provide methyl (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylate (29a). MS (m/z): 1612.3 [M+H]⁺.

Synthesis of (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylic acid (29): To a solution of 29a (0.031 mmol) in THE (0.40 mL) and MeOH (0.40 mL) was added aqueous LiOH (0.6 M, 0.156 mmol) and LiOH (0.094 mmol). The RM was stirred at RT for 1 hour then concentrated in vacuo and purified by RP-HPLC (eluent: water/MeCN 0.1% TFA) to provide (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylic acid (29). MS (m/z): 1598.4 [M+H]⁺. ¹H NMR (400 MHz, Methanol-d4) δ 8.51 (s, 2H), 8.10 (d, J=2.7 Hz, 1H), 7.70-7.34 (m), 7.30-7.25 (m), 7.24 (s, 1H), 7.04-6.98 (m), 6.94 (d, J=2.8 Hz, 1H), 6.72 (s, 1H), 4.99-4.92 (m), 4.86-4.66 (m), 4.52 (s, 1H), 4.34-4.25 (m), 4.23 (d, J=13.4 Hz, 1H), 4.16-4.02 (m), 3.98-3.77 (m), 3.72 (s, 3H), 3.66 (s, 3H), 3.60 (d, J=16.0 Hz, 1H), 3.56-3.41 (m), 3.07-2.97 (m), 2.78 (dd, J=13.2, 6.9 Hz, 1H), 2.69 (d, J=15.9 Hz, 1H), 2.51 (dt, J=13.0, 7.8 Hz, 1H), 2.35 (dd, J=13.5, 6.3 Hz, 1H), 2.30-2.15 (m), 2.07 (s, 3H), 2.01-1.95 (m), 1.80 (s, 3H), 1.61 (s, 3H), 1.23-0.88 (m).

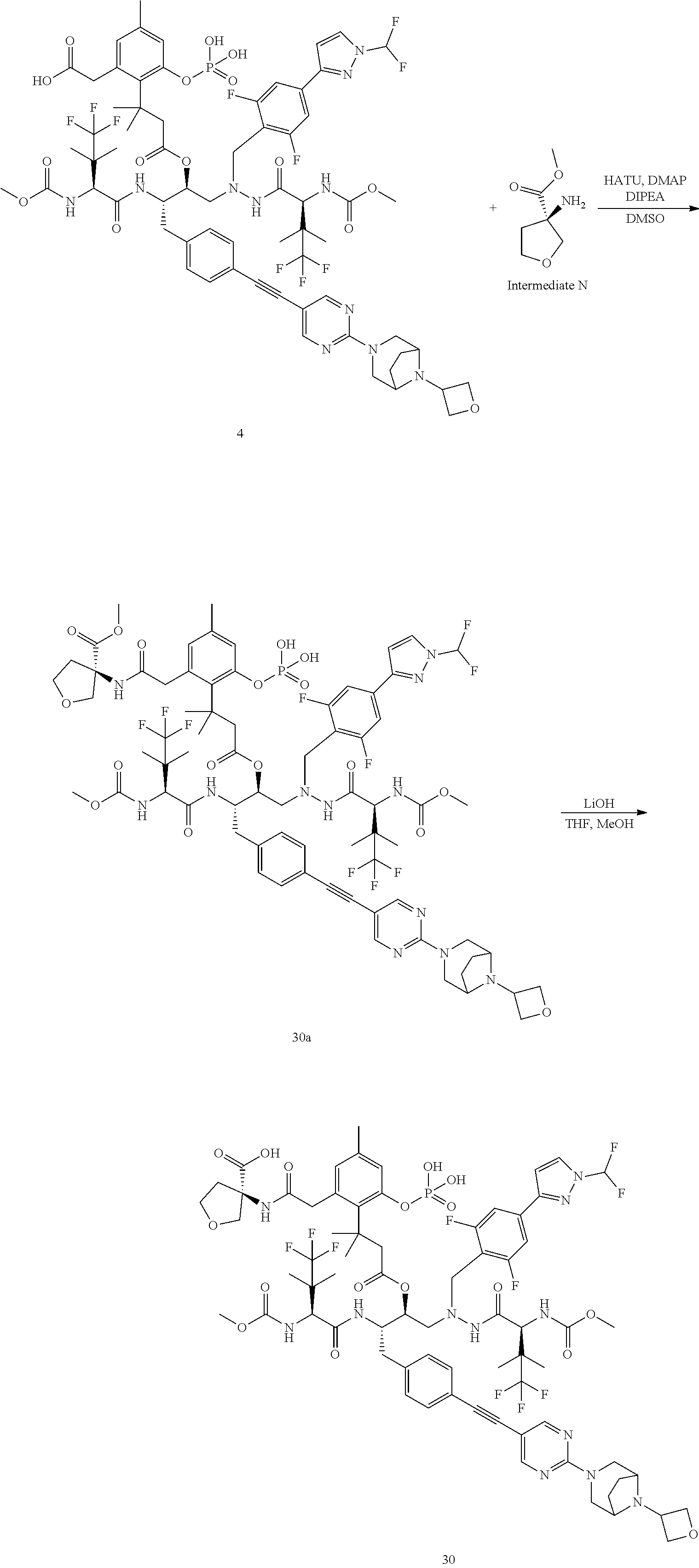

### Example 30

Synthesis of methyl (3R)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylate (30a): Methyl (3R)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylate (30a) was synthesized using the method described in Step 1 of Example 29 substituting Intermediate N for Intermediate M. MS (m/z): 1612.4 [M+H]⁺.

Synthesis of (3R)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylic acid (30): (3R)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydrofuran-3-carboxylic acid (30) was synthesized using the method described in Step 2 of Example 29 substituting 30a for 29A. MS (m/z): 1597.8 [M+H]⁺. ¹H NMR (400 MHz, Methanol-d4) δ 8.52 (s, 2H), 8.10 (d, J=2.7 Hz, 1H), 7.73-7.35 (m), 7.33-7.27 (m), 7.23 (s, 1H), 7.13-7.06 (m), 6.94 (d, J=2.8 Hz, 1H), 6.70 (s, 1H), 5.02-4.92 (m), 4.85-4.66 (m), 4.53 (s, 1H), 4.35 (s, 1H), 4.29-4.10 (m), 4.00-3.86 (m), 3.76 (d, J=17.3 Hz, 1H), 3.72 (s, 3H), 3.66 (s, 3H), 3.56-3.40 (m), 3.01-2.91 (m), 2.83-2.73 (m), 2.48 (dt, J=13.1, 7.7 Hz, 1H), 2.34 (dd, J=13.5, 5.5 Hz, 1H), 2.29-2.18 (m), 2.17-2.08 (m), 2.06 (s, 3H), 2.04-1.94 (m), 1.78 (s, 3H), 1.62 (s, 3H), 1.23-0.91 (m).

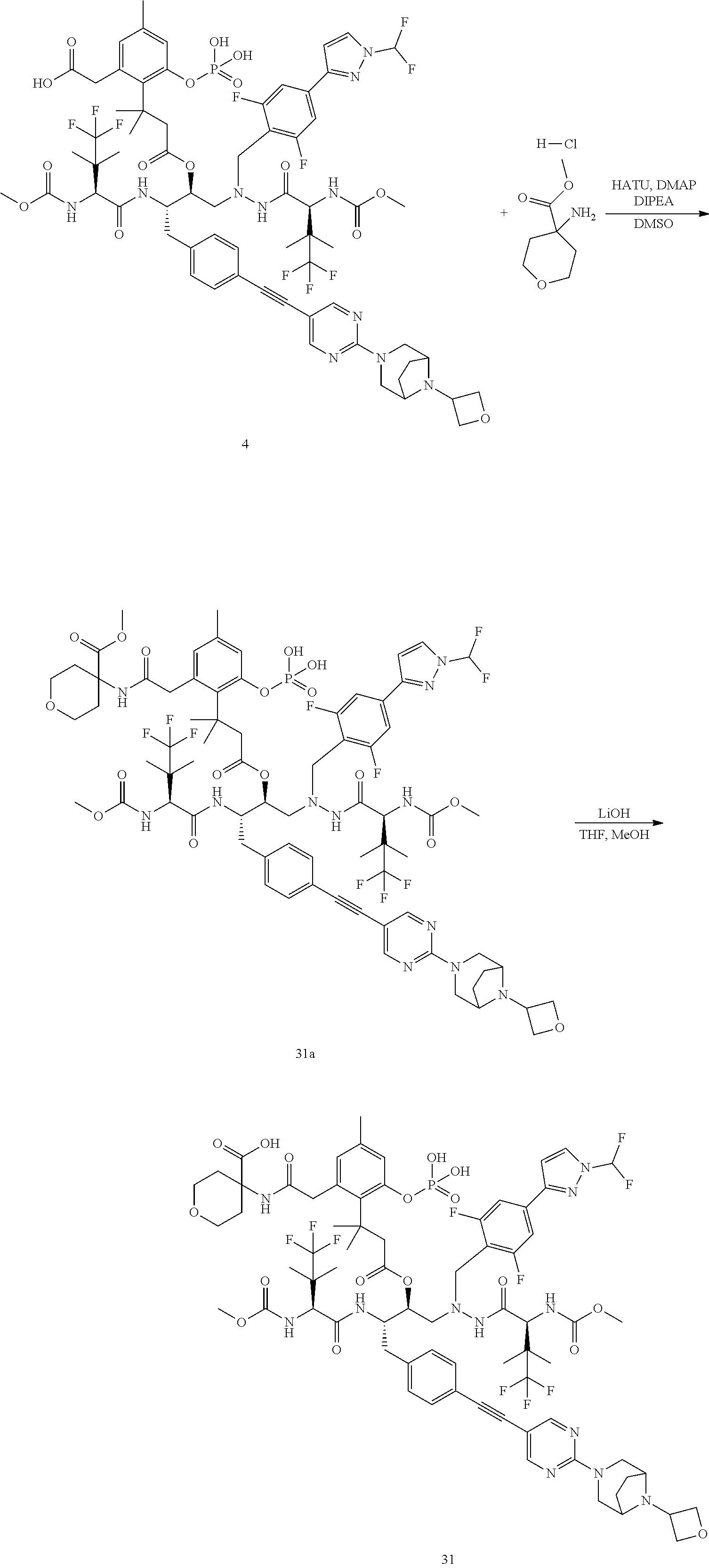

### Example 31

Synthesis of methyl 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydro-2H-pyran-4-carboxylate (31a): Methyl 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydro-2H-pyran-4-carboxylate (31a) was synthesized using the method described in Step 1 of Example 29 substituting methyl 4-aminotetrahydro-2H-pyran-4-carboxylate hydrochloride for Intermediate M. MS (m/z): 1626.3 [M+H]⁺.

Synthesis of 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydro-2H-pyran-4-carboxylic acid (31): 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)tetrahydro-2H-pyran-4-carboxylic acid (31) was synthesized using the method described in Step 2 of Example 29 substituting 31a for 29A. MS (m/z): 1612.5 [M+H]⁺. ¹H NMR (400 MHz, Methanol-d4) δ 8.53 (s, 2H), 8.10 (d, J=2.7 Hz, 1H), 7.75-7.35 (m), 7.30 (s, 1H), 7.28 (s, 1H), 7.24 (s, 1H), 7.10 (s, 1H), 7.08 (s, 1H), 6.96-6.89 (m), 6.74-6.68 (m), 4.96 (t, J=7.6 Hz, 2H), 4.84-4.69 (m), 4.52 (s, 1H), 4.41-4.32 (m), 4.31-4.24 (m), 4.24-4.17 (m), 4.17-4.10 (m), 3.94 (d, J=13.2 Hz, 1H), 3.83-3.74 (m), 3.72 (s, 3H), 3.66 (s, 3H), 3.65-3.58 (m), 3.55-3.44 (m), 3.03-2.92 (m), 2.85-2.70 (m), 2.34 (dd, J=13.7, 5.5 Hz, 1H), 2.26-2.18 (m), 2.18-2.04 (m), 2.04-1.92 (m), 1.79 (s, 3H), 1.62 (s, 3H), 1.24-0.90 (m).

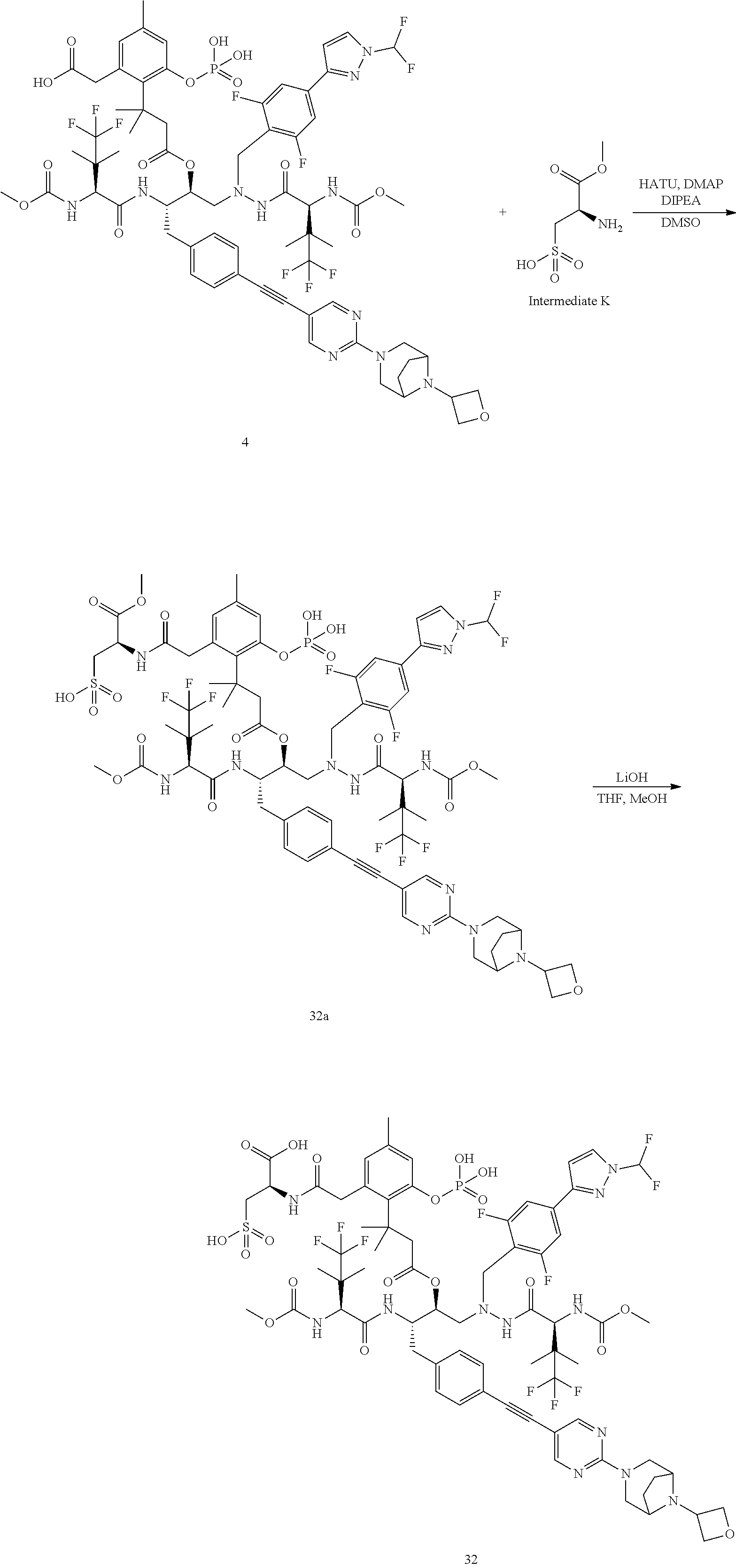

### Example 32

Synthesis of (2R)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-methoxy-3-oxopropane-1-sulfonic acid (32a): (2R)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-methoxy-3-oxopropane-1-sulfonic acid (32a) was synthesized using the method described in Step 1 of Example 29 substituting Intermediate K for Intermediate M. MS (m/z): 1650.5 [M+H]⁺.

Synthesis of (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)(sulfo)-D-alanine (32): (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)(sulfo)-D-alanine (32) was synthesized using the method described in Step 2 of Example 29 substituting 32a for 29A. MS (m/z): 1635.7 [M+H]⁺. ¹H NMR (400 MHz, Methanol-d4) δ 8.51 (s, 2H), 8.10 (d, J=2.8 Hz, 1H), 7.72-7.37 (m), 7.33-7.20 (m), 7.13-7.02 (m), 6.94 (d, J=2.7 Hz, 1H), 6.79 (s, 1H), 5.09-4.92 (m), 4.80-4.70 (m), 4.46 (s, 1H), 4.36 (s, 1H), 4.28 (s, 1H), 4.10-4.25 (m), 3.94 (d, J=13.2 Hz, 1H), 3.82-3.62 (m), 3.54-3.40 (m), 3.29-3.19 (m), 2.99-2.90 (m), 2.82-2.64 (m), 2.34-2.15 (m), 2.09 (s, 3H), 2.00 (s, 2H), 1.86-1.74 (m), 1.63 (s, 3H), 1.21-0.87 (m).

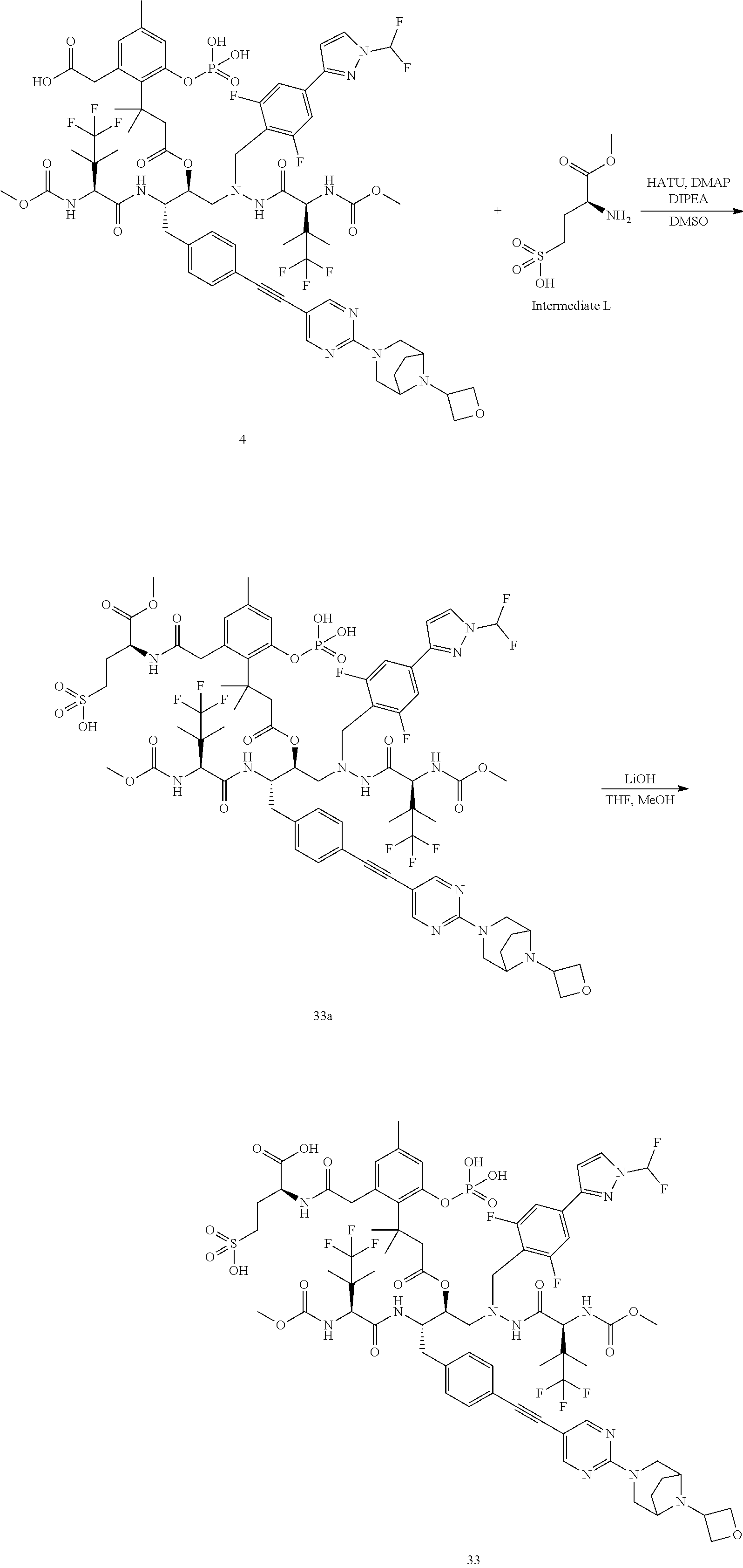

### Example 33

Synthesis of (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-methoxy-4-oxobutane-1-sulfonic acid (33a): (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-methoxy-4-oxobutane-1-sulfonic acid (33a) was synthesized using the method described in Step 1 of Example 29 substituting Intermediate L for Intermediate M. MS (m/z): 1664.4 [M+H]⁺.

Synthesis of (2S)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-sulfobutanoic acid (33): (2S)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-sulfobutanoic acid (33) was synthesized using the method described in Step 2 of Example 29 substituting 33a for 29A. MS (m/z): 1649.5 [M+H]⁺. ¹H NMR (400 MHz, Methanol-d4) δ 8.52 (s, 2H), 8.10 (d, J=2.7 Hz, 1H), 7.71-7.36 (m), 7.29 (d, J=7.8 Hz, 2H), 7.22 (s, 1H), 7.08 (d, J=7.8 Hz, 2H), 6.95 (d, J=2.8 Hz, 1H), 6.78 (s, 1H), 4.97 (dd, J=7.6 Hz, 2H), 4.83-4.74 (m), 4.60 (dd, J=8.7, 4.8 Hz, 1H), 4.47 (s, 1H), 4.40-4.32 (m), 4.32-4.22 (m), 4.19 (s, 1H), 4.17-4.10 (m), 3.92 (d, J=13.4 Hz, 1H), 3.77 (d, J=17.2 Hz, 1H), 3.72 (s, 3H), 3.66-3.65 (m), 3.51-3.47 (m), 3.45 (s, 1H), 3.40-3.34 (m), 3.01-2.80 (m), 2.78-2.66 (m), 2.47-2.33 (m), 2.31-2.15 (m), 2.09 (s, 3H), 2.02-1.96 (m), 1.77 (s, 3H), 1.64 (s, 3H), 1.25-0.81 (m).

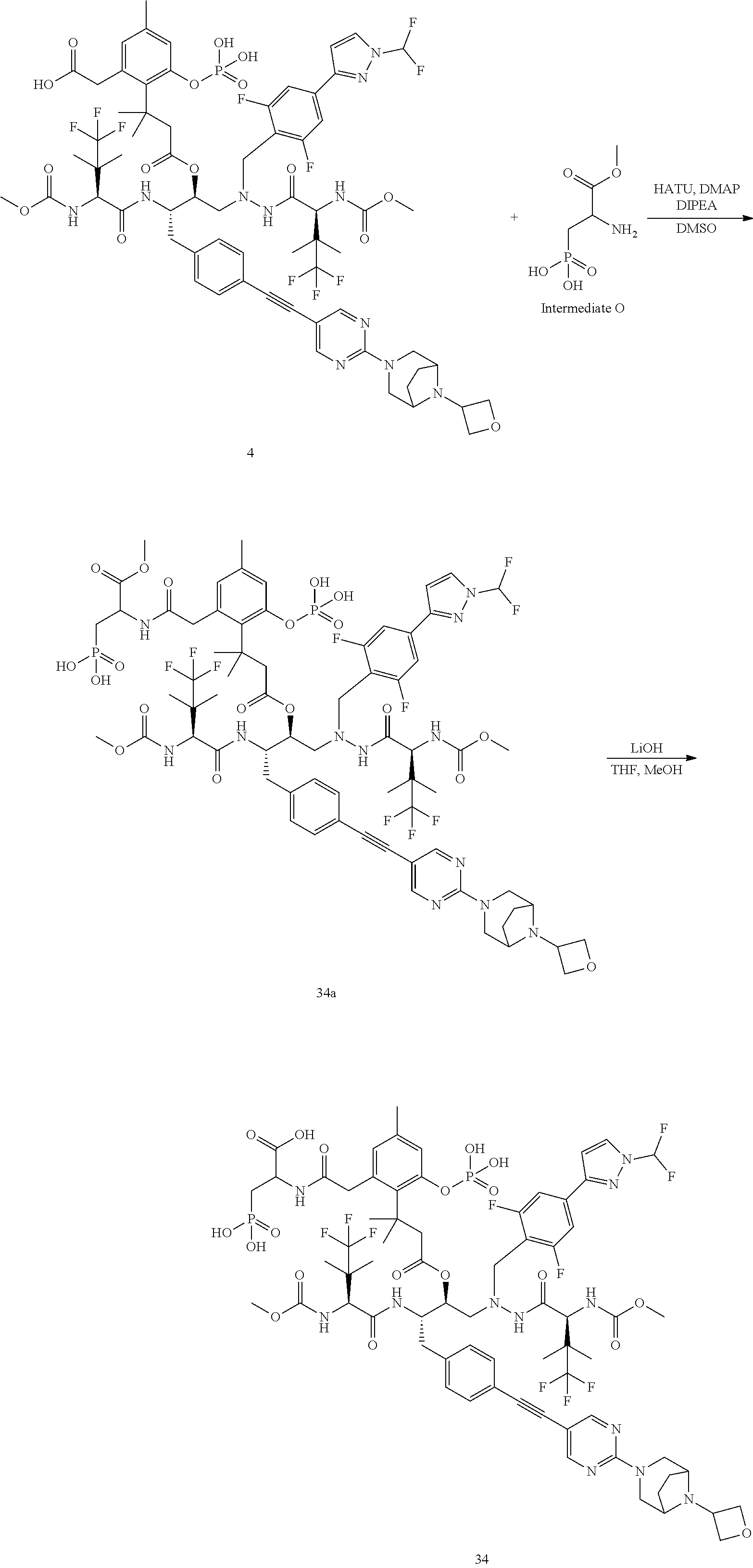

### Example 34

Synthesis of (2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-methoxy-3-oxopropyl)phosphonic acid (34a): (2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-methoxy-3-oxopropyl)phosphonic acid (34a) was synthesized as a mixture of diastereomers using the method described in Step 1 of Example 29 substituting Intermediate O for Intermediate M. MS (m/z): 1650.1 [M+H]⁺.

Synthesis of 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-phosphonopropanoic acid (34): 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-phosphonopropanoic acid (34) was synthesized as a mixture of diastereomers using the method described in Step 2 of Example 29 substituting 34a for 29A. MS (m/z): 1636.1 [M+H]⁺. ¹H NMR (400 MHz, Methanol-d4) δ 8.55 (s, 2H), 8.12 (d, J=2.7 Hz, 1H), 7.74-7.37 (m), 7.36-7.23 (m), 7.17-7.04 (m), 6.96 (d, J=2.7 Hz, 1H), 6.92 (d, J=10.1 Hz, 1H), 6.75 (s, 1H), 4.98 (t, J=7.6 Hz, 2H), 4.84-4.63 (m), 4.54 (s, 1H), 4.38 (s, 1H), 4.33-4.24 (m), 4.23-4.11 (m), 3.96 (d, J=13.2 Hz, 1H), 3.80-3.65 (m), 3.61-3.42 (m), 3.06-2.91 (m), 2.83-2.72 (m), 2.39-2.18 (m), 2.15-1.92 (m), 1.84-1.76 (m), 1.69-1.60 (m), 1.25-0.90 (m).

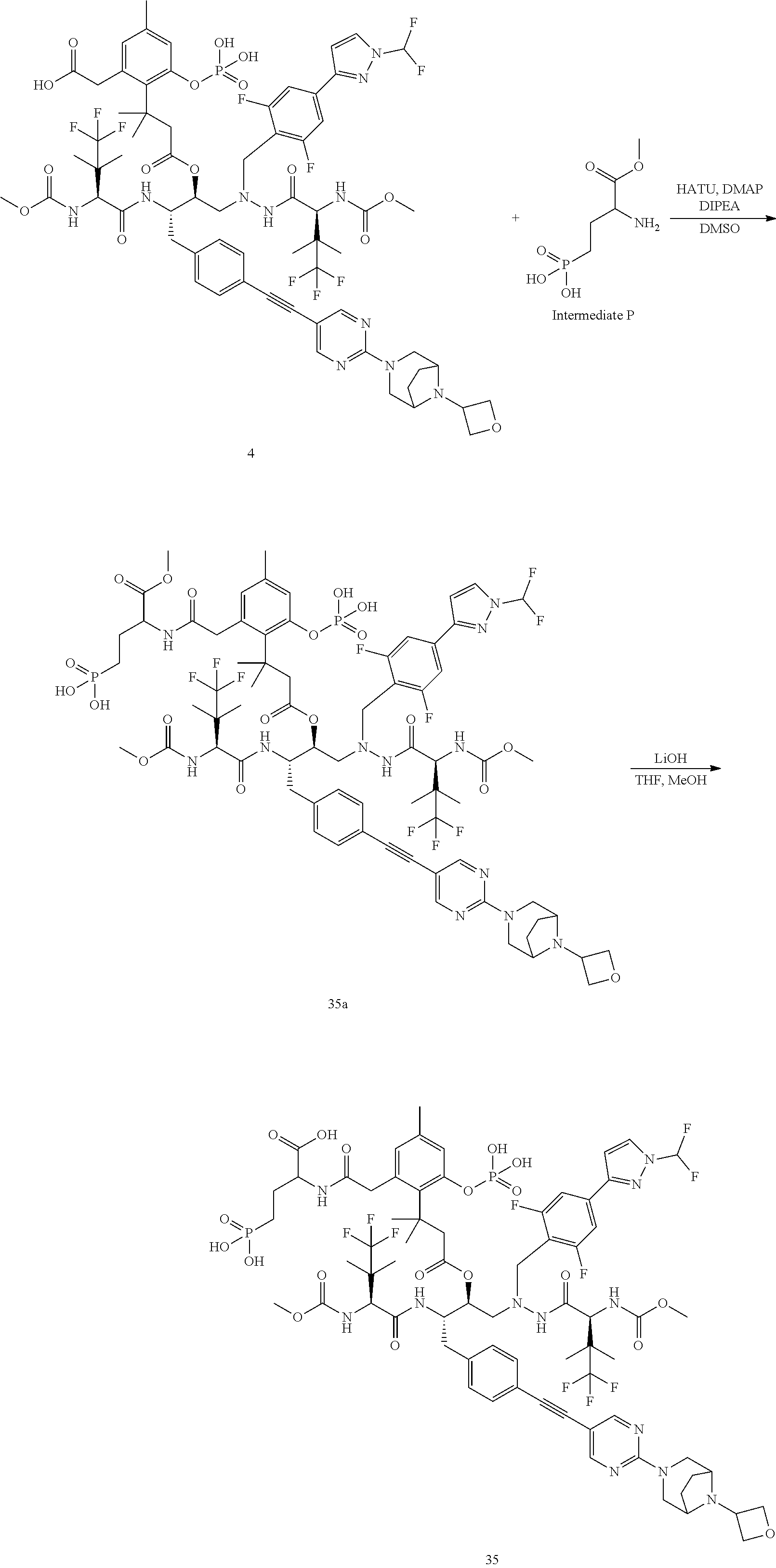

### Example 35

Synthesis of (3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-methoxy-4-oxobutyl)phosphonic acid (35a): (3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-methoxy-4-oxobutyl)phosphonic acid (35a) was synthesized as a mixture of diastereomers using the method described in Step 1 of Example 29 substituting Intermediate P for Intermediate M. MS (m/z): 1664.2 [M+H]⁺.

Synthesis of 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-phosphonobutanoic acid (35): 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-4-phosphonobutanoic acid (35) was synthesized as a mixture of diastereomers using the method described in Step 2 of Example 29 substituting 35a for 29A. MS (m/z): 1649.4 [M+H]⁺. ¹H NMR (400 MHz, Methanol-d4) δ 8.53 (d, J=0.8 Hz, 2H), 8.10 (d, J=2.7 Hz, 1H), 7.73-7.36 (m), 7.30 (d, J=7.9 Hz, 2H), 7.25 (s, 1H), 7.14-7.06 (m), 6.94 (d, J=2.8 Hz, 1H), 6.93-6.86 (m), 6.74-6.67 (m), 4.99-4.93 (m), 4.84-4.72 (m), 4.60-4.47 (m), 4.39-4.30 (m), 4.28 (dd, J=10.1, 3.6 Hz, 1H), 4.26-4.09 (m), 3.93 (d, J=13.3 Hz, 1H), 3.81-3.69 (m), 3.66 (s, 3H), 3.60-3.54 (m), 3.55-3.40 (m), 3.04-2.90 (m), 2.90-2.81 (m), 2.81-2.71 (m), 2.35-2.12 (m), 2.12-1.91 (m), 1.82-1.69 (m), 1.67-1.59 (m), 1.23-0.88 (m).

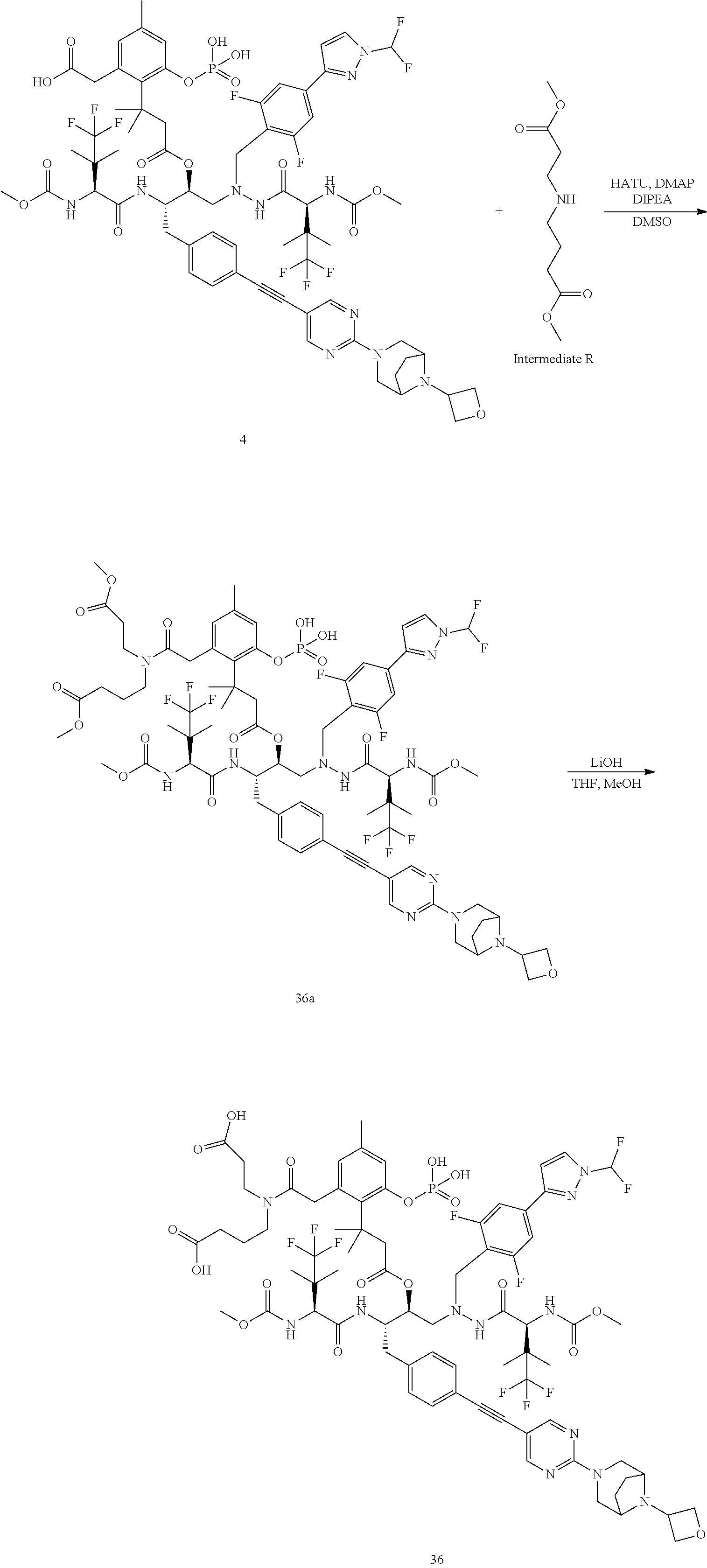

### Example 36

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-methoxy-3-oxopropyl)(4-methoxy-4-oxobutyl)amino)-2-oxoethyl)-4-methyl-6-(phosphonooxy)phenyl)-3-methylbutanoate (36a): (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-methoxy-3-oxopropyl)(4-methoxy-4-oxobutyl)amino)-2-oxoethyl)-4-methyl-6-(phosphonooxy)phenyl)-3-methylbutanoate (36a) was synthesized using the method described in Example 27 substituting Intermediate R for Intermediate Q. MS (m/z): 1669.6 [M+H]⁺.

Synthesis of 4-(N-(2-carboxyethyl)-2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)butanoic acid (36): 4-(N-(2-carboxyethyl)-2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)butanoic acid (36) was synthesized using the method described in Step 2 of Example 29 substituting 36a for 29A. MS (m/z): 1642.4 [M+H]⁺. ¹H NMR (400 MHz, Methanol-d4) δ 8.56-8.47 (m), 8.15-8.07 (m), 7.71-7.37 (m), 7.32-7.26 (m), 7.23 (s), 7.14-7.06 (m), 6.97-6.91 (m), 6.60-6.53 (m), 5.01-4.92 (m), 4.85-4.72 (m), 4.53 (s, 1H), 4.42-4.09 (m), 4.04-3.82 (m), 3.76-3.58 (m), 3.56-3.36 (m), 3.00-2.81 (m), 2.80-2.57 (m), 2.41-2.27 (m), 2.26-2.17 (m), 2.11-1.82 (m), 1.77-1.68 (m), 1.61 (s, 3H), 1.23-0.86 (m).

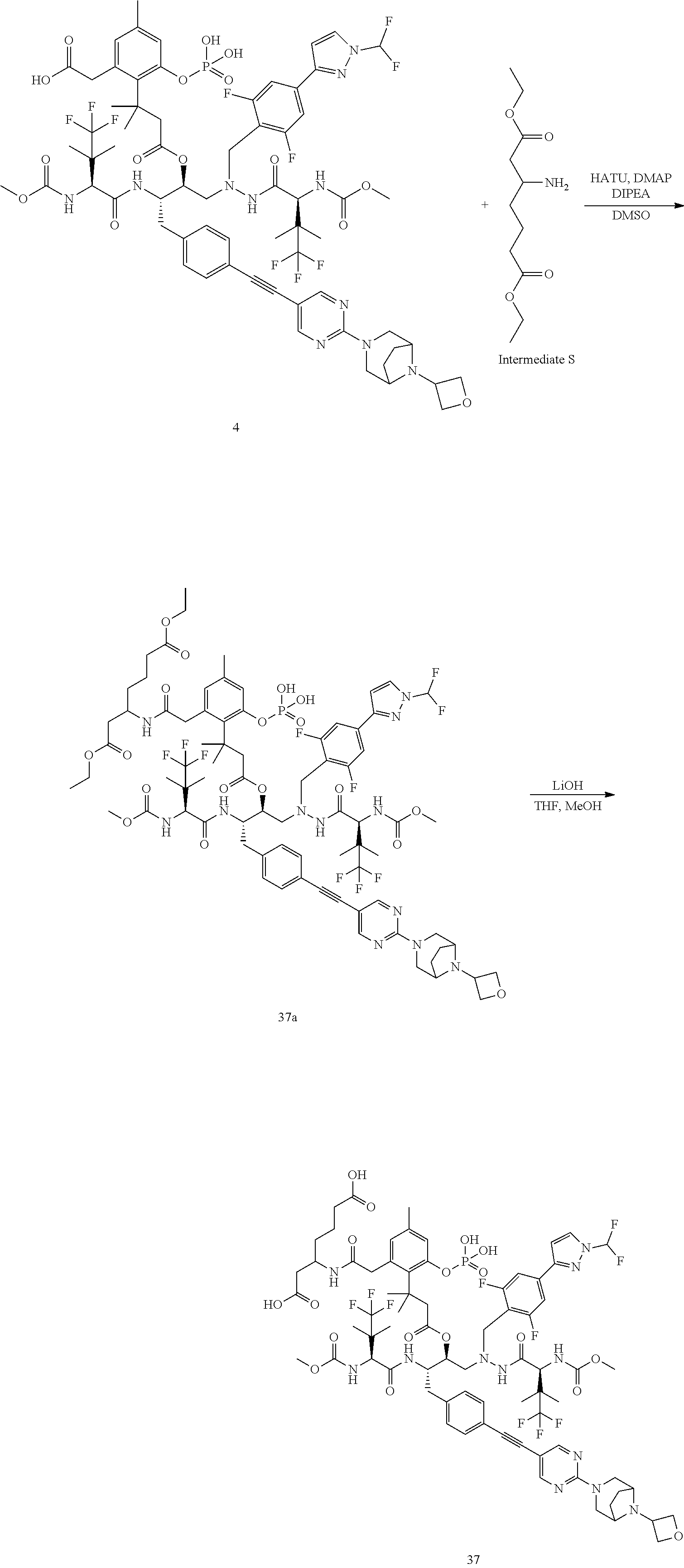

### Example 37

Synthesis of 1 diethyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)heptanedioate (37a): Diethyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)heptanedioate (37a) was synthesized as a mixture of diastereomers using the method described in Example 27 substituting Intermediate S for Intermediate Q. MS (m/z): 1698.4 [M+H]⁺.

Synthesis of 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)heptanedioic acid (37): 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)heptanedioic acid (37) was synthesized as a mixture of diastereomers using the method described in Step 2 of Example 29 substituting 37a for 29A. MS (m/z): 1642.2 [M+H]⁺. ¹H NMR (400 MHz, Methanol-d4) δ 8.52 (s, 2H), 8.10 (d, J=2.8 Hz, 1H), 7.78-7.36 (m), 7.34-7.26 (m), 7.24 (s, 1H), 7.14-7.06 (m), 6.94 (d, J=2.8 Hz, 1H), 6.89 (d, J=10.1 Hz, 1H), 6.79-6.66 (m), 5.01-4.94 (m), 4.74-4.85 (m), 4.52 (s, 1H), 4.35 (s, 1H), 4.30-4.18 (m), 4.19-4.05 (m), 3.98-3.87 (m), 3.80-3.61 (m), 3.53-3.36 (m), 3.04-2.91 (m), 2.90-2.71 (m), 2.56-2.44 (m), 2.37-2.25 (m), 2.26-2.17 (m), 2.16-2.04 (m), 2.05-1.94 (m), 1.76 (s, 3H), 1.71-1.52 (m), 1.24-0.89 (m).

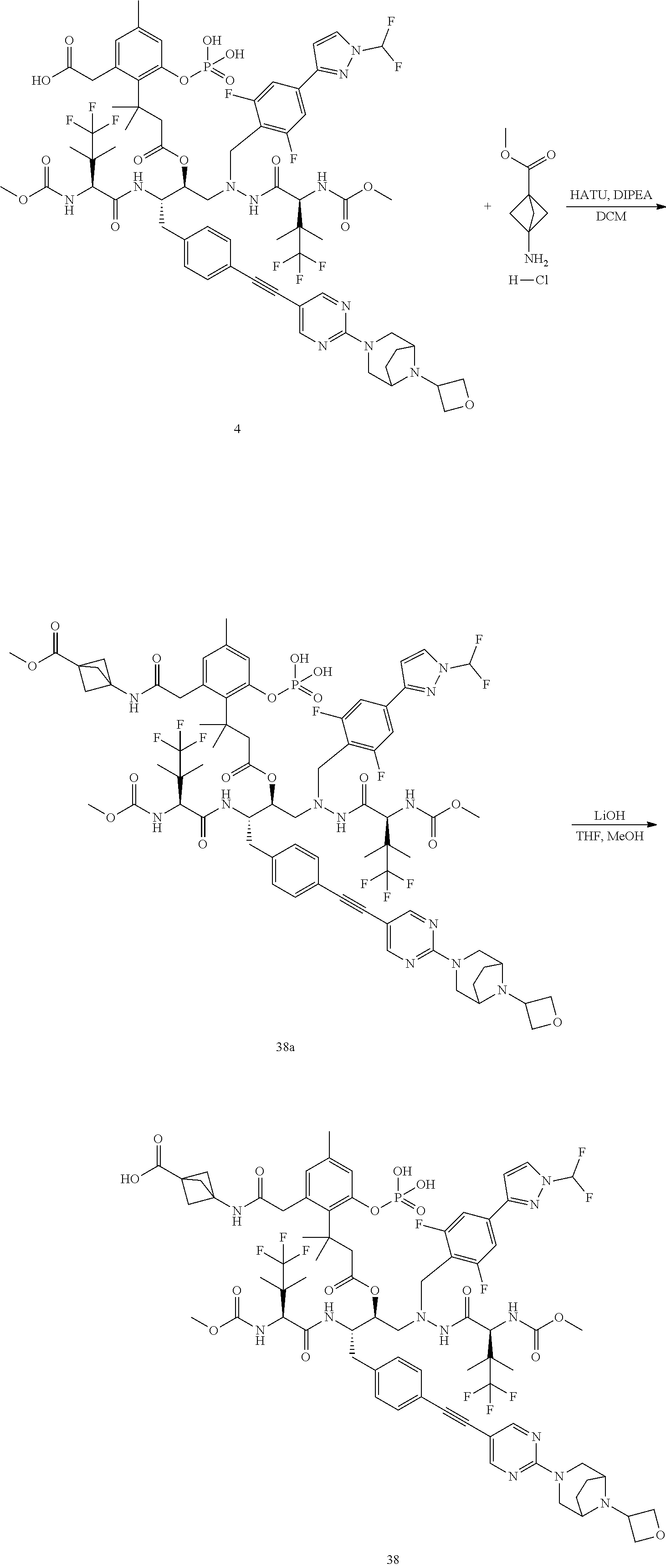

### Example 38

Synthesis of methyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)bicyclo[1.1.1]pentane-1-carboxylate (38a): To a solution of 4 (0.031 mmol), and methyl 3-aminobicyclo[1.1.1]pentane-1-carboxylate hydrochloride (0.066 mmol) in DCM (1.0 mL) was added DIPEA (0.22 mmol) followed by HATU (0.034 mmol). The RM was stirred at RT for 15 min then concentrated in vacuo and the resulting residue purified by RP-HPLC (eluent: water/MeCN 0.1% TFA) to provide methyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)bicyclo[1.1.1]pentane-1-carboxylate (38a). MS (m/z): 1608.6 [M+H]⁺.

Synthesis of 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)bicyclo[1.1.1]pentane-1-carboxylic acid (38): 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)bicyclo[1.1.1]pentane-1-carboxylic acid (38) was synthesized using the method described in Step 2 of Example 29 substituting 38a for 29A. MS (m/z): 1593.9 [M+H]⁺. ¹H NMR (400 MHz, Methanol-d4) δ 8.53 (s, 2H), 8.10 (d, J=2.7 Hz, 1H), 7.71-7.37 (m), 7.35-7.22 (m), 7.09 (d, J=7.9 Hz, 2H), 6.94 (d, J=2.7 Hz, 1H), 6.90 (d, J=10.2 Hz, 1H), 6.72 (d, J=10.1 Hz, 1H), 6.63 (s, 1H), 4.96 (t, J=7.6 Hz, 2H), 4.85-4.69 (m), 4.52 (s, 1H), 4.39-4.30 (m), 4.27 (d, J=10.1 Hz, 1H), 4.23-4.11 (m), 4.05 (d, J=17.3 Hz, 1H), 3.93 (d, J=13.3 Hz, 1H), 3.81-3.60 (m), 3.57-3.42 (m), 3.04-2.92 (m), 2.85-2.72 (m), 2.29 (s, 6H), 2.25-2.17 (m), 2.14-2.01 (m), 2.05-1.94 (m), 1.74 (s, 3H), 1.61 (s, 3H), 1.24-0.91 (m).

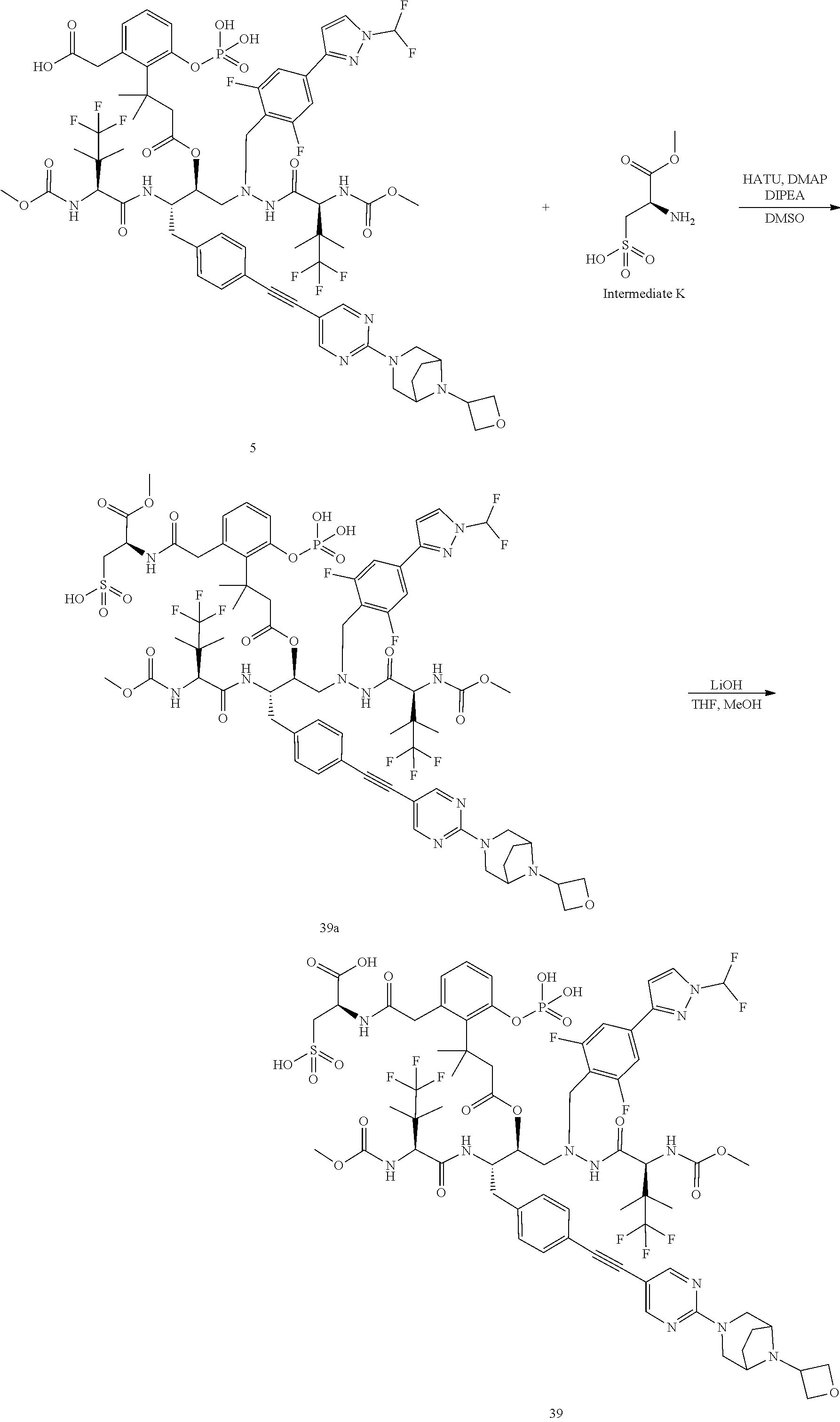

### Example 39

Synthesis of (2R)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)-3-methoxy-3-oxopropane-1-sulfonic acid (39a): (2R)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)-3-methoxy-3-oxopropane-1-sulfonic acid (39a) was synthesized using the method described in Step 1 of Example 29 substituting 5 for 4 and substituting Intermediate K for Intermediate M. MS (m/z): 1636.3 [M+H]⁺.

Synthesis of (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)(sulfo)-D-alanine (39): (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)(sulfo)-D-alanine (39) was synthesized using the method described in Step 2 of Example 29 substituting 39a for 29A. MS (m/z): 1621.7 [M+H]⁺. ¹H NMR (400 MHz, Methanol-d4) δ 8.52 (s, 2H), 8.10 (d, J=2.7 Hz, 1H), 7.71-7.36 (m), 7.32-7.26 (m), 7.13-7.04 (m), 7.00 (d, J=7.7 Hz, 1H), 6.94 (d, J=2.8 Hz, 1H), 6.85 (d, J=9.7 Hz, 1H), 5.03-4.93 (m), 4.84-4.67 (m), 4.55-4.37 (m), 4.32-4.25 (m), 4.21 (d, J=16.8 Hz, 1H), 4.18-4.08 (m), 3.93 (d, J=13.3 Hz, 1H), 3.82 (d, J=17.8 Hz, 1H), 3.72 (s, 3H), 3.65 (s, 3H), 3.53-3.37 (m), 3.29-3.21 (m), 3.00-2.90 (m), 2.90-2.80 (m), 2.80-2.64 (m), 2.42-2.31 (m), 2.27-2.16 (m), 2.04-1.96 (m), 1.80 (s, 3H), 1.66 (s, 3H), 1.25-0.89 (m).

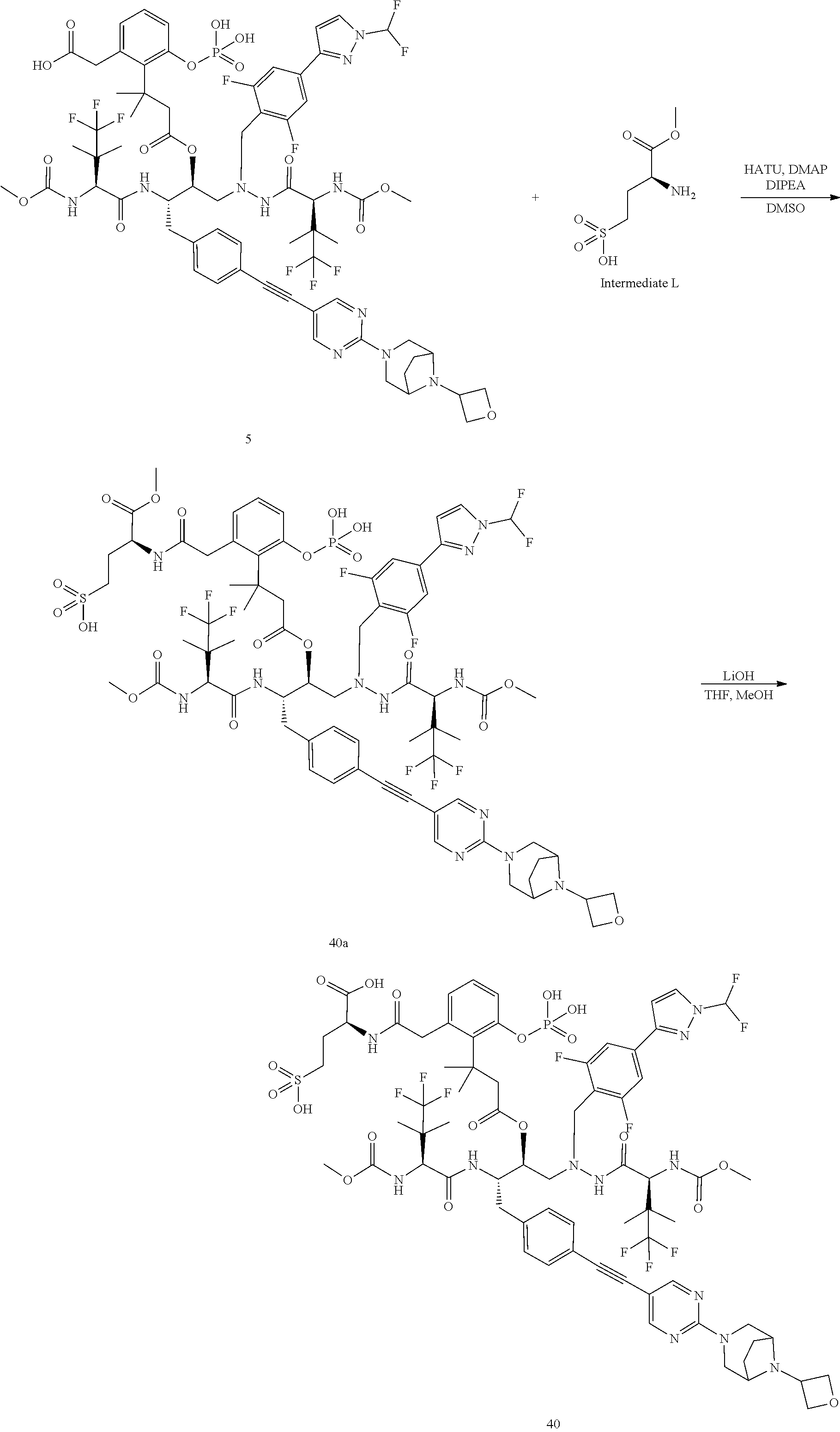

### Example 40

Synthesis of (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)-4-methoxy-4-oxobutane-1-sulfonic acid (40a): (3S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)-4-methoxy-4-oxobutane-1-sulfonic acid (40a) was synthesized using the method described in Step 1 of Example 29 substituting 5 for 4 and substituting Intermediate L for Intermediate M. MS (m/z): 1650.3 [M+H]⁺.

Synthesis of (2S)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)-4-sulfobutanoic acid (40): (2S)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-3-(phosphonooxy)phenyl)acetamido)-4-sulfobutanoic acid (40) was synthesized using the method described in Step 2 of Example 29 substituting 40a for 29A. MS (m/z): 1636.1 [M+H]⁺. ¹H NMR (400 MHz, Methanol-d4) δ 8.51 (s, 2H), 8.10 (d, J=2.7 Hz, 1H), 7.72-7.36 (m), 7.30 (s, 1H), 7.28 (s, 1H), 7.14-7.03 (m), 6.98 (d, J=7.9 Hz, 1H), 6.95 (d, J=2.8 Hz, 1H), 4.96 (t, J=7.6 Hz, 2H), 4.85-4.70 (m), 4.60 (dd, J=8.7, 4.8 Hz, 1H), 4.51-4.37 (m), 4.31-4.21 (m), 4.22-4.15 (m), 4.14 (s, 3H), 3.96-3.83 (m), 3.72 (s, 3H), 3.65 (s, 3H), 3.52-3.48 (m), 3.45 (s, 1H), 3.00-2.82 (m), 2.73-2.64 (m), 2.48-2.12 (m), 1.99 (d, J=9.1 Hz, 2H), 1.77 (s, 3H), 1.67 (s, 3H), 1.26-0.87 (m).

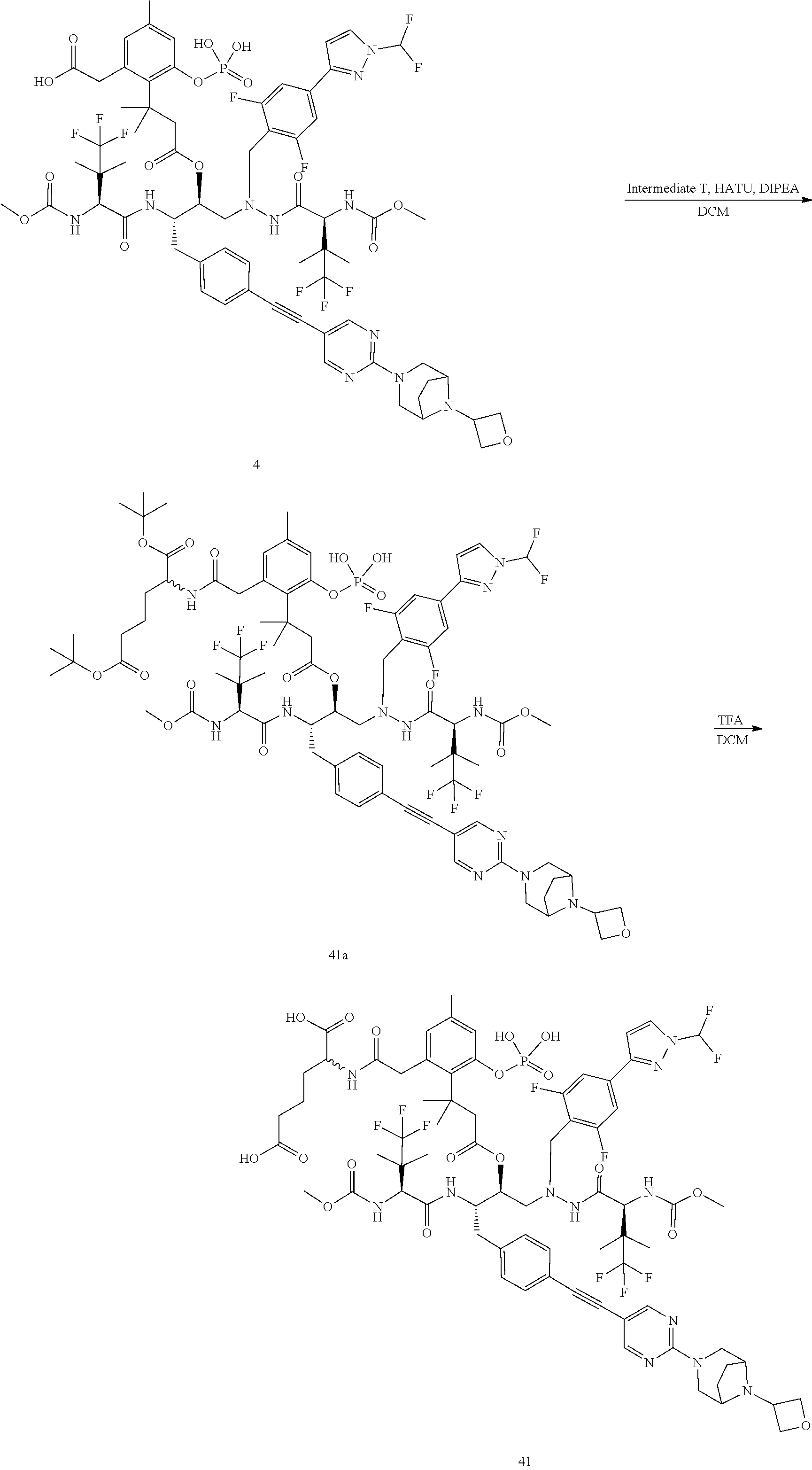

### Example 41

Synthesis of di-tert-butyl 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)hexanedioate (41a): Di-tert-butyl 2-aminohexanedioate Intermediate T (0.0146 mmol) was added to a stirring solution of N,N-diisopropylethylamine (0.0876 mmol) and 4 (0.0365 mmol) in DCM (2.0 mL) at room temperature, followed by HATU (0.0511 mmol) and stirred for 30 min. The reaction mixture was concentrated and purified by RP-HPLC (eluent: water/MeCN 0.1% TFA) to give 41a. MS (m/z): 870.5 [M+2H]²⁺. ¹H NMR (400 MHz, Methanol-d4) δ 8.55 (s, 2H), 8.12 (d, J=2.7 Hz, 1H), 7.71-7.38 (m), 7.36-7.24 (m), 7.12 (d, J=8.0 Hz, 2H), 6.99-6.88 (m), 6.75 (s, 1H), 4.99 (t, J=7.6 Hz, 2H), 4.57-4.12 (m), 3.94 (m), 3.71 (m), 3.55-3.38 (m), 3.05-2.73 (m), 2.37-2.19 (m), 2.18-1.97 (m), 1.72 (m), 1.46 (m), 1.24-1.12 (m), 1.00 (m).

Synthesis of 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)hexanedioic acid (41): TFA (2.0 mL) was added to a stirring solution of 41a (0.00920 mmol) in DCM (2.0 mL) at room temperature and stirred for 45 min. The solution was concentrated and the product isolated by RP-HPLC (water/MeCN 0.1% TFA) to give 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)hexanedioic acid 41. MS (m/z): 815.8 [M+2H]²⁺. ¹H NMR (400 MHz, Methanol-d₄) δ 8.55 (s, 2H), 8.12 (d, J=2.7 Hz, 1H), 7.75-7.38 (m), 7.36-7.24 (m), 7.12 (d, J=7.9 Hz, 2H), 7.00-6.86 (m), 6.76 (s, 1H), 4.99 (m), 4.51 (m), 4.42-4.12 (m), 3.94 (d, J=13.4 Hz, 1H), 3.71 (m), 3.56-3.39 (m), 2.84 (m), 2.38-1.89 (m), 1.72 (m), 1.31 (m), 1.24-1.11 (m), 1.00 (m). ¹⁹F NMR (376 MHz, Methanol-d4) δ −77.19 (s), −77.85 (m), −96.95 (m), −114.40 (m). ³¹P NMR (162 MHz, Methanol-d₄) δ −6.89 (s).

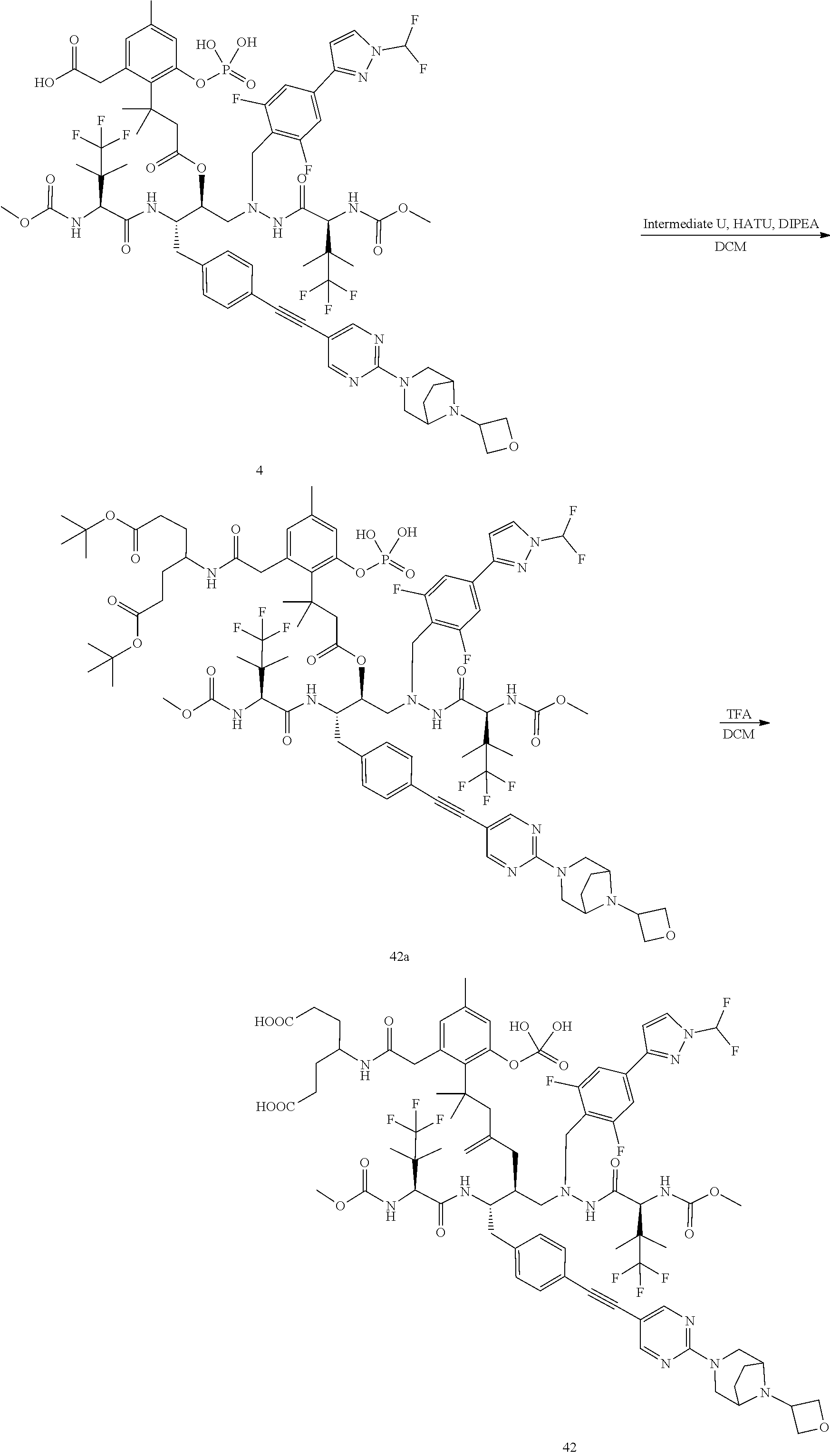

### Example 42

Synthesis of di-tert-butyl 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)heptanedioate (42a): Di-tert-butyl 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)heptanedioate (42a) was synthesized using the method described in Example 41: Step 1, substituting di-tert-butyl 4-aminoheptanedioate Intermediate U for Intermediate T. MS (m/z): 870.5 [M+2H]²⁺. ¹H NMR (400 MHz, Methanol-d4) δ 8.55 (s, 2H), 8.12 (d, J=2.7 Hz, 1H), 7.71-7.38 (m), 7.36-7.24 (m), 7.12 (d, J=8.0 Hz, 2H), 6.99-6.88 (m), 6.75 (s, 1H), 4.99 (t, J=7.6 Hz, 2H), 4.57-4.12 (m), 3.94 (m), 3.71 (m), 3.55-3.38 (m), 3.05-2.73 (m), 2.37-2.19 (m), 2.18-1.97 (m), 1.72 (m), 1.46 (m), 1.24-1.12 (m), 1.00 (m).

Synthesis of 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)hexanedioic acid (42): 2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)hexanedioic acid (42) was synthesized using the method described in Example 41: Step 2, substituting Intermediate 42a for Intermediate 41a. MS (m/z): 821.2 [M+2H]²⁺. ¹H NMR (400 MHz, Methanol-d₄) δ 8.55 (s, 2H), 8.12 (d, J=2.8 Hz, 1H), 7.73-7.37 (m), 7.36-7.20 (m), 7.13 (m), 6.99-6.85 (m), 6.73 (m), 4.99 (t, J=7.6 Hz, 2H), 4.59-4.27 (m), 4.17 (m), 3.95 (m), 3.71 (m), 3.54-3.40 (m), 3.07-2.74 (m), 2.41-1.59 (m), 1.20 (m), 1.00 (s, 3H). ¹⁹F NMR (376 MHz, Methanol-d₄) δ −77.16 (s), −77.78 (m), −96.94 (m), −114.48 (m). ³¹P NMR (162 MHz, Methanol-d₄) δ −6.89.

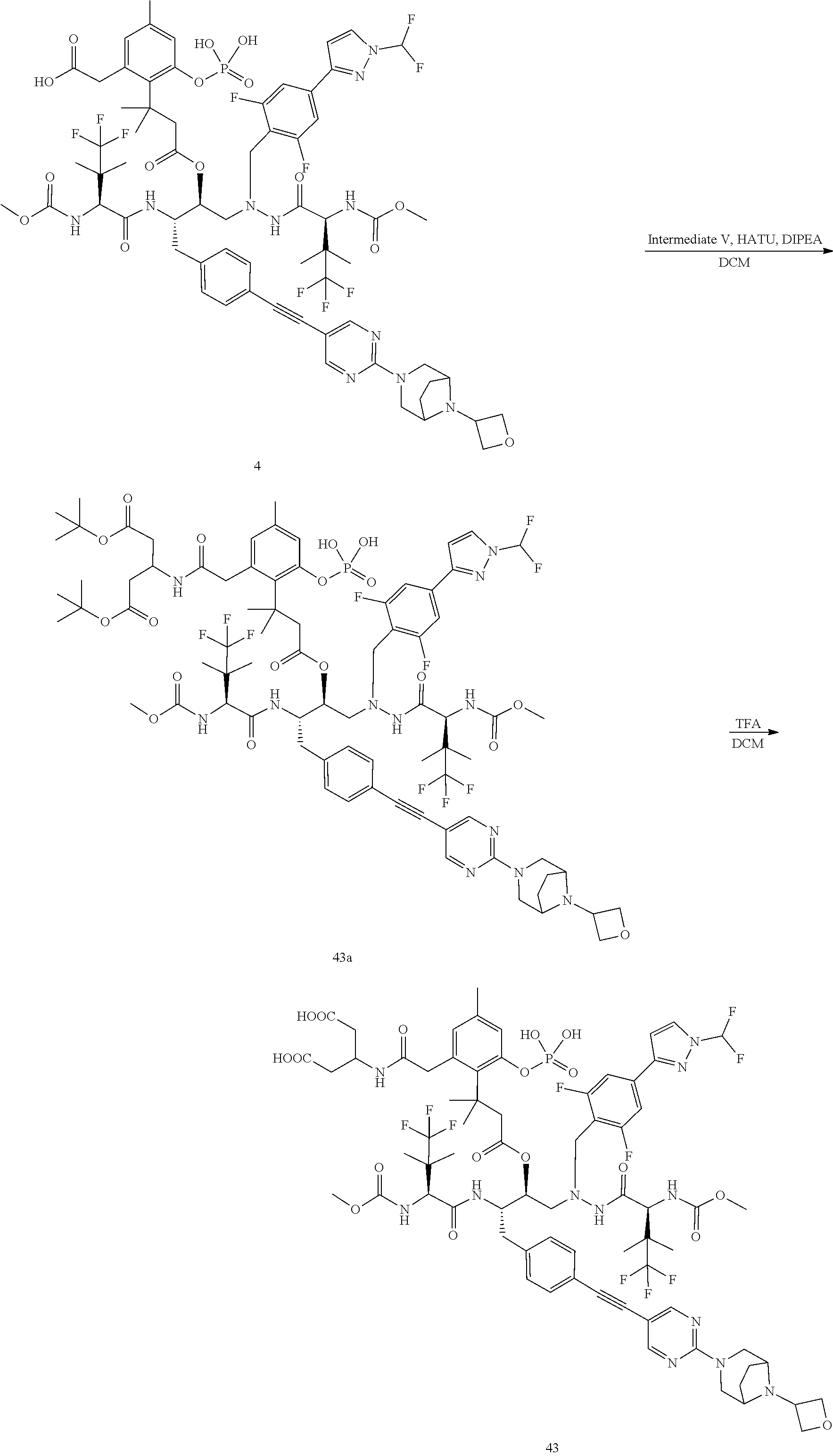

### Example 43

Synthesis of di-tert-butyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)pentanedioate (43a): Di-tert-butyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)pentanedioate (43a) was synthesized using the method described in Example 41: Step 1, substituting di-tert-butyl 3-aminopentanedioate Intermediate V for Intermediate T. MS (m/z): 864.759 [M+2H]²⁺. ¹H NMR (400 MHz, Methanol-d₄) δ 8.55 (s, 2H), 8.12 (d, J=2.7 Hz, 1H), 7.73-7.38 (m), 7.31 (m), 7.12 (d, J=7.8 Hz, 2H), 7.00-6.87 (m), 6.67 (m), 4.99 (t, J=7.5 Hz, 2H), 4.63-3.90 (m), 3.71 (m), 3.55-3.45 (m), 2.84 (m), 2.51 (d, J=6.5 Hz, 3H), 2.39-1.98 (m), 1.77 (s, 3H), 1.64 (s, 3H), 1.44 (s, 18H), 1.20 (m), 1.00 (s, 3H).

Synthesis of 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)pentanedioic acid (43): 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)pentanedioic acid (43) was synthesized using the method described in Example 41: Step 2, substituting 43a for 41a. MS (m/z): 807.2 [M+2H]²⁺. ¹H NMR (400 MHz, Methanol-d₄) δ 8.55 (s, 2H), 8.12 (d, J=2.7 Hz, 1H), 7.73-7.38 (m), 7.36-7.23 (m), 7.12 (d, J=7.9 Hz, 2H), 7.01-6.85 (m), 6.70 (s, 1H), 4.99 (t, J=7.6 Hz, 2H), 4.60 (m), 4.40-4.26 (m), 4.25-4.06 (m), 3.95 (d, J=13.2 Hz, 1H), 3.71 (m), 3.56-3.39 (m), 3.00 (m), 2.83 (m), 2.64 (d, J=6.5 Hz, 4H), 2.27 (m), 2.16-1.97 (m), 1.76 (s, 3H), 1.63 (s, 3H), 1.24-1.10 (m), 1.00 (s, 3H). ¹⁹F NMR (376 MHz, Methanol-d₄) δ −77.20 (s), −77.81 (m), −96.95 (m), −114.40 (m). ³¹P NMR (162 MHz, Methanol-d₄) δ −6.91 (s).

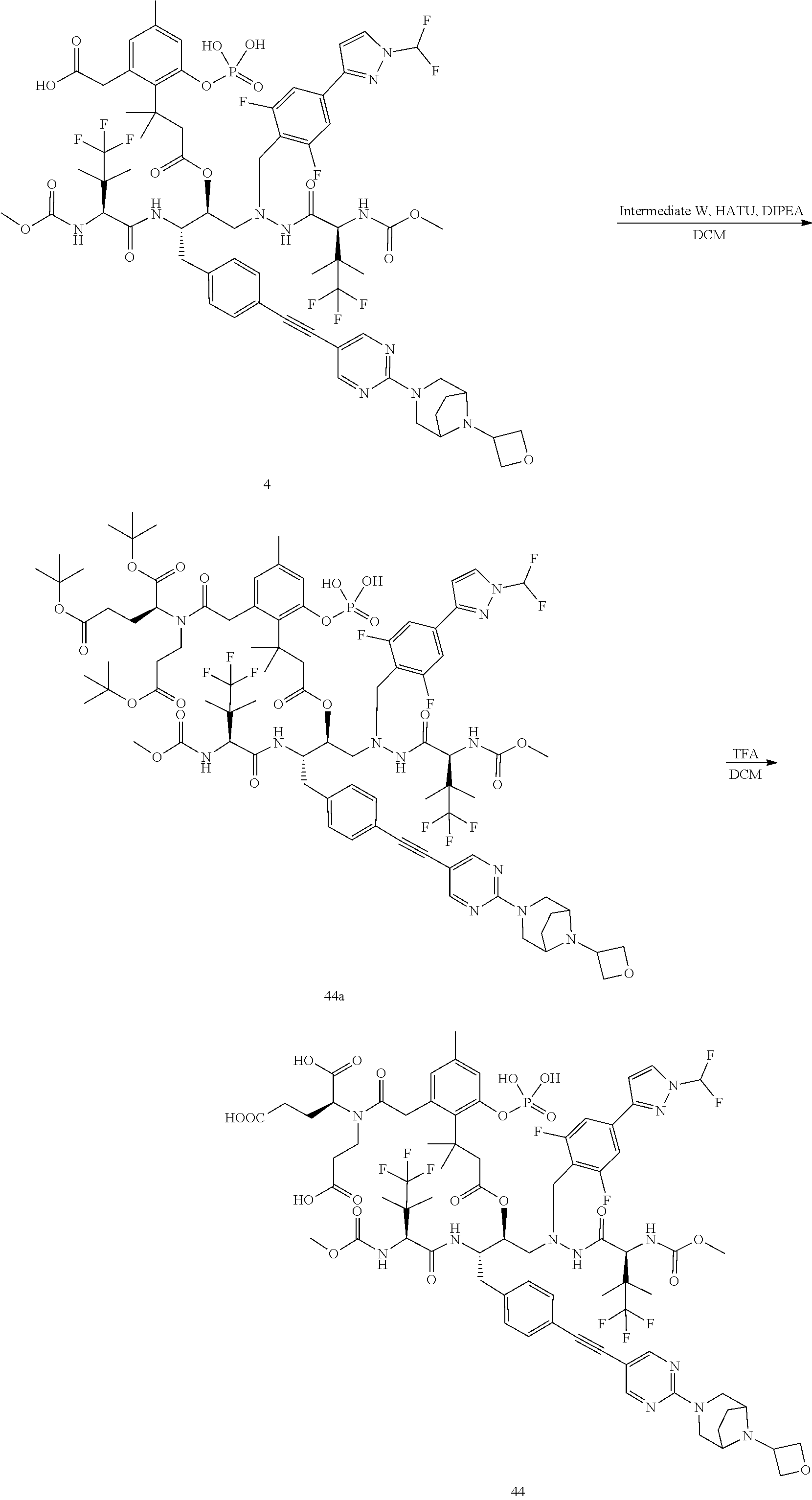

### Example 44

Synthesis of di-tert-butyl N-(3-(tert-butoxy)-3-oxopropyl)-N-(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamate (44a): di-tert-butyl N-(3-(tert-butoxy)-3-oxopropyl)-N-(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamate (44a) was synthesized using the method described in Example 41: Step 1, substituting di-tert-butyl (3-(tert-butoxy)-3-oxopropyl)-L-glutamate Intermediate W for Intermediate T. MS (m/z): 927.460 [M+2H]²⁺

Synthesis of N-(2-carboxyethyl)-N-(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamic acid (44): N-(2-carboxyethyl)-N-(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamic acid (44) was synthesized using the method described in Example 41: Step 2, substituting 44a for 41a. MS (m/z): 844.412 [M+2H]²⁺. ¹H NMR (400 MHz, Methanol-d₄) δ 8.55 (s, 2H), 8.12 (d, J=2.7 Hz, 1H), 7.73-7.38 (m), 7.36-7.22 (m), 7.12 (m), 7.00-6.85 (m), 6.61 (m), 4.99 (t, J=7.6 Hz, 2H), 4.58-4.28 (m), 4.18 (m), 4.00 (m), 3.77-3.63 (m), 3.56-3.46 (m), 3.03-2.67 (m), 2.51-1.98 (m), 1.80 (s, 2H), 1.73 (s, 1H), 1.62 (m), 1.27-1.09 (m), 1.03 (m). ¹⁹F NMR (376 MHz, Methanol-d₄) δ −77.20 (s), −77.72 (m), −96.95 (m), −114.36 (m). ³¹P NMR (162 MHz, Methanol-d₄) δ −7.04 (s).

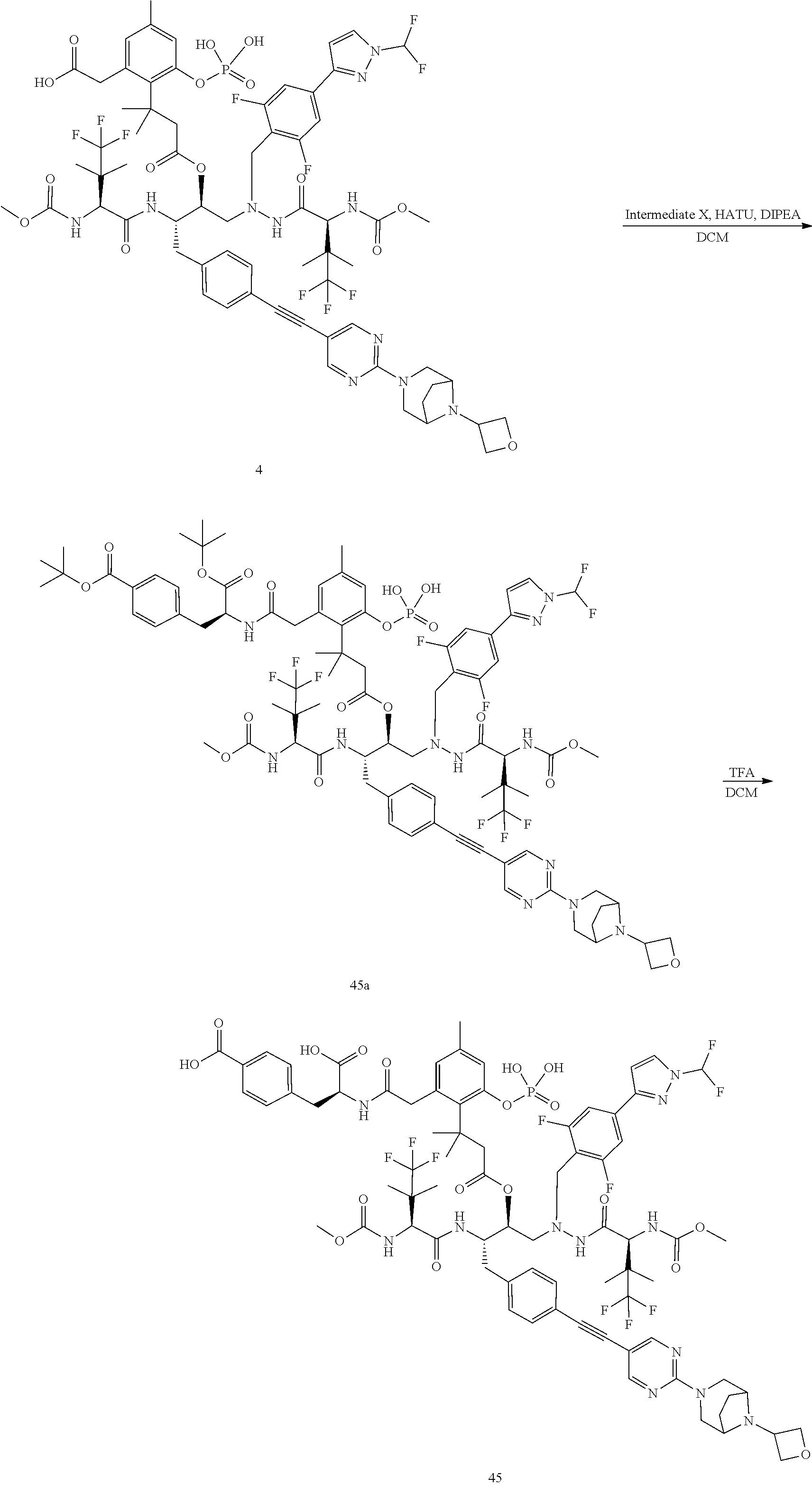

### Example 45

Synthesis of tert-butyl 4-((2S)-3-(tert-butoxy)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-oxopropyl)benzoate (45a): Tert-butyl 4-((2S)-3-(tert-butoxy)-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-3-oxopropyl)benzoate (45a) was synthesized using the method described in Example 41: Step 1, substituting tert-butyl (S)-4-(2-amino-3-(tert-butoxy)-3-oxopropyl)benzoate Intermediate X for Intermediate T. MS (m/z): 894.300 [M+2H]²⁺

Synthesis of Step 2: 4-((2S)-2-carboxy-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)ethyl)benzoic acid (45): 4-((2S)-2-carboxy-2-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)ethyl)benzoic acid 45 was synthesized using the method described in Example 41: Step 2, substituting 45a for 41a. MS (m/z): 838.359 [M+2H]²⁺. ¹H NMR (400 MHz, Methanol-d₄) δ 8.55 (s, 2H), 8.12 (d, J=2.8 Hz, 1H), 7.94 (d, J=8.1 Hz, 2H), 7.72-7.37 (m), 7.35-7.22 (m), 7.10 (d, J=8.0 Hz, 2H), 6.98-6.86 (m), 6.57 (s, 1H), 4.99 (t, J=7.5 Hz, 2H), 4.40-4.26 (m), 4.16 (m), 3.94 (d, J=13.5 Hz, 1H), 3.80-3.58 (m), 3.56-3.39 (m), 3.09 (dd, J=13.9, 8.8 Hz, 1H), 2.96 (m), 2.74 (m), 2.24 (m), 2.00 (m), 1.58 (m), 1.37-1.09 (m), 1.00 (m). ¹⁹F NMR (376 MHz, Methanol-d₄) δ −77.18 (s), −77.85 (m), −96.94 (m), −114.40 (m). ³¹P NMR (162 MHz, Methanol-d4) δ −6.97 (s).

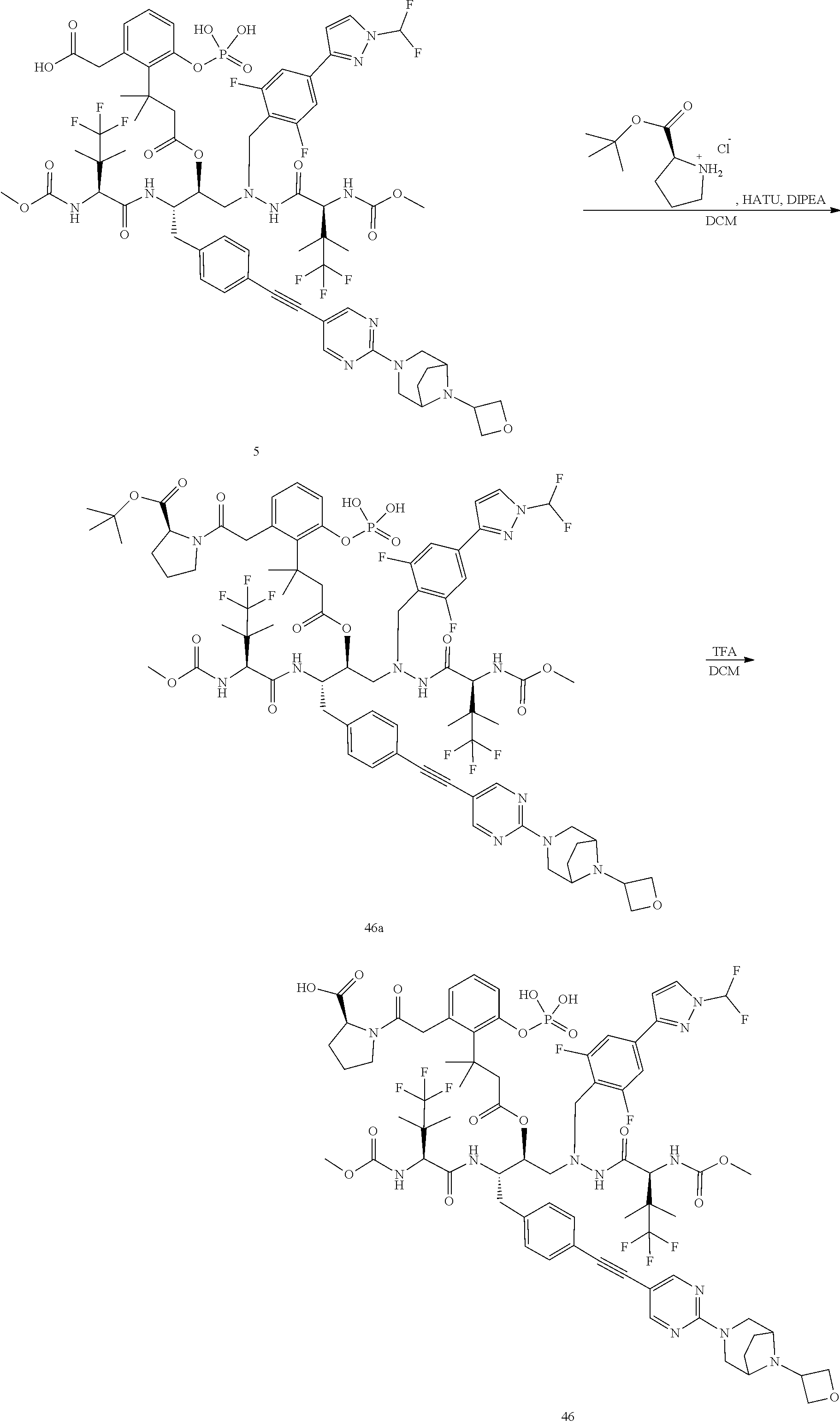

### Example 46

Synthesis of tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-prolinate (46a): tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-prolinate (46a) was synthesized using the method described in Example 41: Step 1, substituting tert-butyl L-prolinate hydrochloride for Intermediate T and substituting 5 for 4. MS (m/z): 812.382 [M+2H]²⁺

Synthesis of Step 2: (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-proline (46): (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-3-(phosphonooxy)phenyl)acetyl)-L-proline (46) was synthesized using the method described in Example 41: Step 2, substituting 46a for 41a. MS (m/z): 784.200 [M+2H]²⁺. ¹H NMR (400 MHz, Methanol-d₄) δ 8.53 (s, 2H), 8.13 (d, J=2.7 Hz, 1H), 7.74-7.39 (m), 7.29 (d, J=7.6 Hz, 2H), 7.18-7.02 (m), 6.99-6.72 (m), 4.89 (m), 4.63-4.37 (m), 4.34-3.90 (m), 3.82-3.57 (m), 3.51 (d, J=14.5 Hz, 2H), 3.40 (d, J=15.7 Hz, 1H), 2.95 (d, J=15.7 Hz, 2H), 2.71 (m), 2.49-2.17 (m), 2.10-1.92 (m), 1.77-1.56 (m), 1.19 (m), 0.98 (m). ¹⁹F NMR (376 MHz, Methanol-d₄) δ −77.20 (s), −77.81 (s), −96.85-−97.07 (m), −114.27 (m). P NMR (162 MHz, Methanol-d4) δ −7.06 (s).

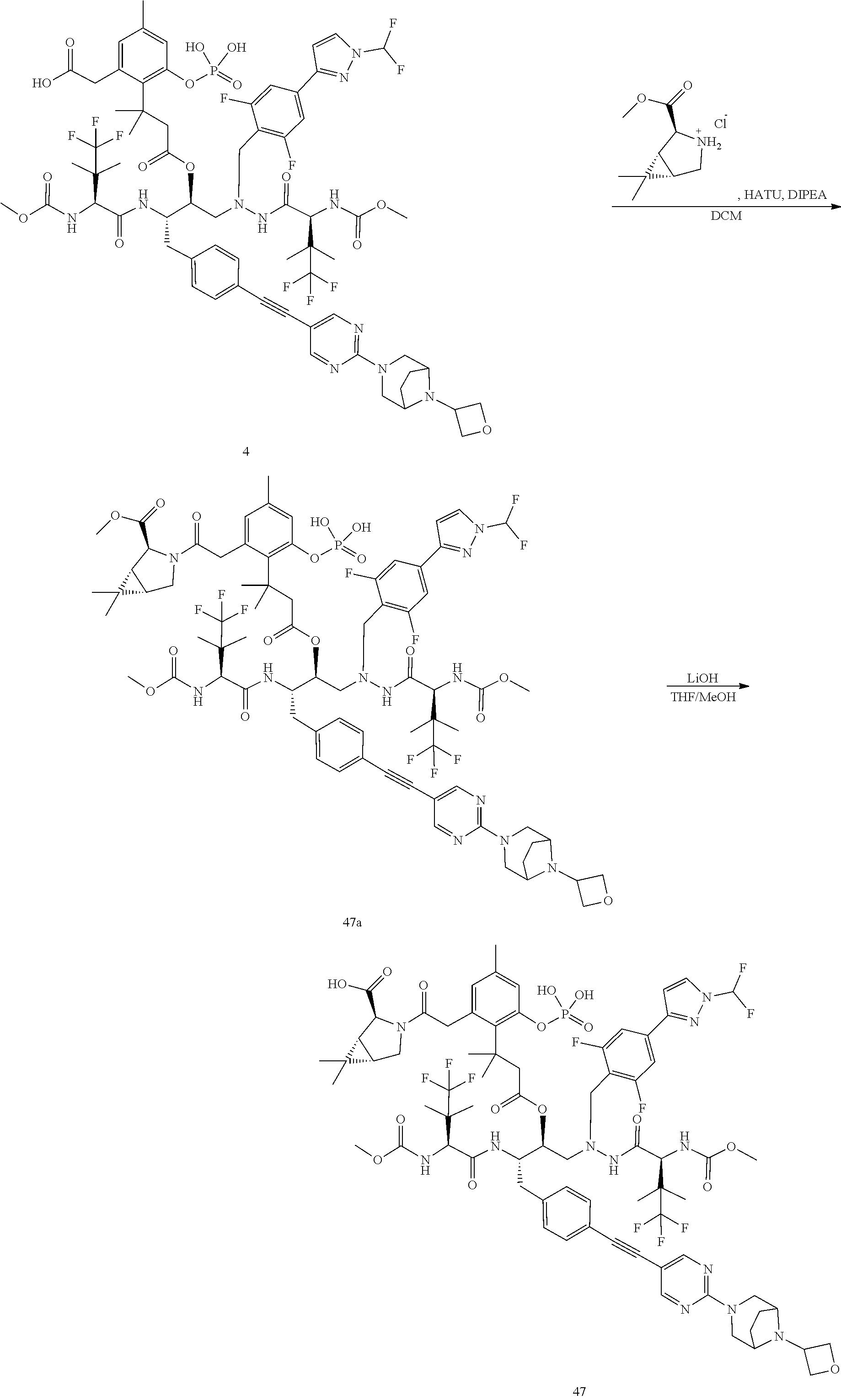

### Example 47

Synthesis of methyl (1R,2S,5S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-6,6-dimethyl-3-azabicyclo[3.1.0]hexane-2-carboxylate (47a): methyl (1R,2S,5S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-6,6-dimethyl-3-azabicyclo[3.1.0]hexane-2-carboxylate (47a) was synthesized using the method described in Example 41: Step 1, substituting (1R,2S,5S)-methyl 6,6-dimethyl-3-azabicyclo[3.1.0]hexane-2-carboxylate hydrochloride for Intermediate T. MS (m/z): 818.281 [M+2H]²⁺

Synthesis of (1R,2S,5S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-6,6-dimethyl-3-azabicyclo[3.1.0]hexane-2-carboxylic acid (47): Methyl (1R,2S,5S)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-6,6-dimethyl-3-azabicyclo[3.1.0]hexane-2-carboxylate 47a (0.138 mmol) was dissolved in a mixture of THE (5.0 mL) and methanol (5.0 mL) at room temperature. Aqueous lithium hydroxide (0.6 M, 1.85 mL) was added, and the mixture was stirred vigorously for 18 hr. Acetic acid (1.11 mmol) was then added, and the reaction mixture was concentrated. The crude residue was purified by RP-HPLC (eluent: water/MeCN 0.1% TFA) to give 47. MS (m/z): 811.425 [M+2H]²⁺. ¹H NMR (400 MHz, Methanol-d₄) δ 8.55 (s, 2H), 8.13 (d, J=2.7 Hz, 1H), 7.73-7.37 (m), 7.35-7.21 (m), 7.10 (m), 7.02-6.88 (m), 6.66 (m), 4.88-4.75 (m), 4.61-4.10 (m), 4.03-3.44 (m), 2.96 (m), 2.73 (m), 2.42-1.91 (m), 1.79-1.47 (m), 1.39-0.85 (m). ¹⁹F NMR (376 MHz, Methanol-d₄) δ −77.16 (s), −77.74 (s), −96.94 (m), −114.43 (m). ³¹P NMR (162 MHz, Methanol-d₄) δ −7.05 (s).

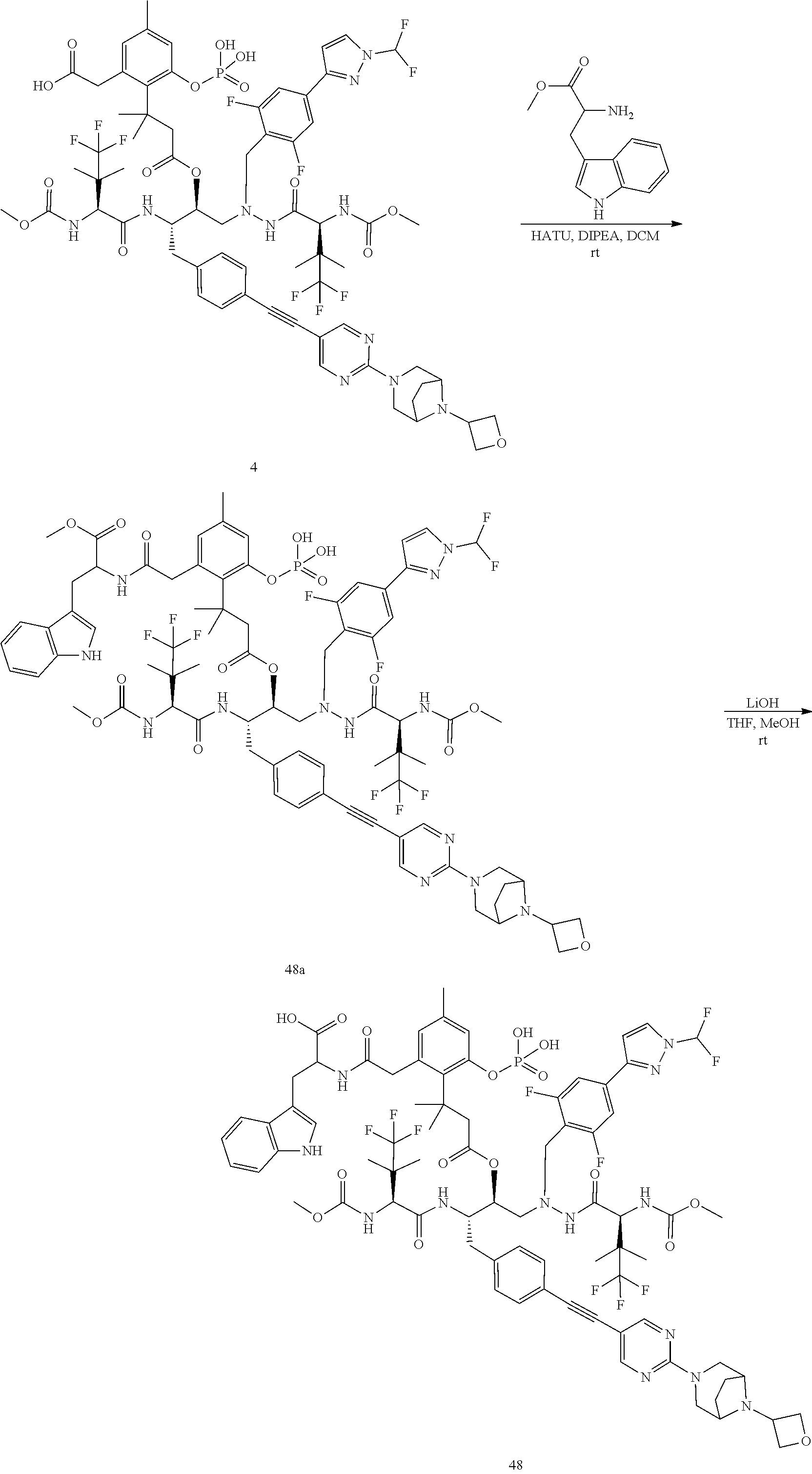

### Example 48

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a): To a solution of 4 (0.148 mmol) and methyl 2-amino-3-(1H-indol-3-yl)propanoate (0.296 mmol) in dichloromethane (1.48 mL), N, N, N-diisopropylethylamine (DIPEA) (0.16 mL) and HATU (0.222 mmol) were added in a sequential manner and the mixture was stirred at rt for 1 hour. The mixture was quenched by addition of 0.25 mL trifluoroacetic acid (TFA) and concentrated under reduced pressure. Reverse phase preparative HPLC employing water/MeCN as the eluent with 0.1% TFA modifier afforded (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). MS (m/z): 843.01 [M+2H]²⁺.

Synthesis of (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)tryptophan (48): To a solution of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a) (0.086 mmol) in THE (0.86 mL) and methanol (0.43 mL), 1.0 M lithium hydroxide solution (0.86 mL) was added in a drop wise manner and the mixture was stirred at rt for 1.5 hours. The mixture was quenched by addition of 0.25 mL TFA and concentrated under reduced pressure. Reverse phase preparative HPLC employing water/MeCN as the eluent with 0.1% TFA modifier afforded (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)tryptophan (48). m/z): 836.11 [M+2H]²⁺. ¹H NMR (400 MHz, Methanol-d₄) δ 8.62-8.53 (m), 8.11 (d, J=2.7 Hz, 1H), 7.72-7.38 (m), 7.38-7.28 (m), 7.13-7.03 (m), 7.02-6.88 (m), 6.82-6.67 (m), 6.43-6.29 (m), 5.02-4.94 (m), 4.79-4.64 (m), 4.39-4.25 (m), 4.23-4.09 (m), 3.93 (m), 3.73 (s, 3H), 3.67 (s, 3H), 3.63-3.47 (m), 3.27 (d, J=5.9 Hz, 2H), 3.03-2.90 (m), 2.83-2.72 (m), 2.60 (d, J=15.5 Hz, 1H), 2.30-2.16 (m), 2.09-1.96 (m), 1.94-1.80 (m), 1.68-1.56 (m), 1.41 (s, 2H), 1.24-1.10 (m), 1.03-0.90 (m). ¹⁹F NMR (376 MHz, DMSO-d₆) δ −74.80 (s), −75.33 (m), −95.06 (d, J=20.2 Hz), −112.80 (s). ³¹P NMR (162 MHz, DMSO-d₆) δ −8.03 (s).

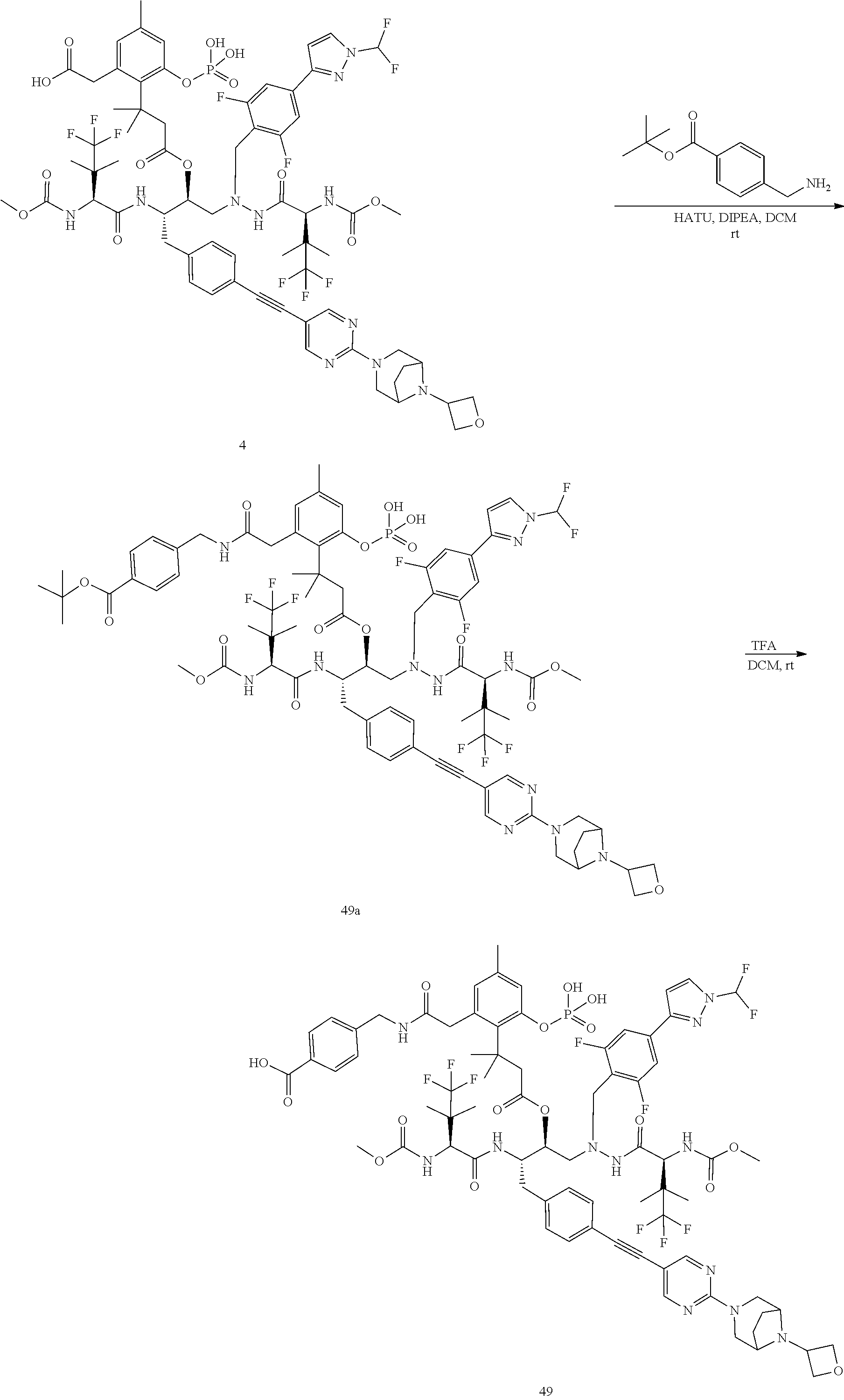

### Example 49

Synthesis of tert-butyl 4-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (49a): To a solution of 4 (0.148 mmol) and tert-butyl 4-(aminomethyl)benzoate (0.296 mmol) in dichloromethane (1.48 mL) N, N, N-diisopropylethylamine (DIPEA) (0.16 mL) and HATU (0.222 mmol) were added in a sequential manner and the mixture was stirred at rt for 1 hour. The mixture was quenched by addition of 0.25 mL trifluoroacetic acid (TFA) and concentrated under reduced pressure. Reverse phase preparative HPLC employing water/MeCN as the eluent with 0.1% TFA modifier afforded tert-butyl 4-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (49a). MS (m/z): 837.43 [M+2H]²⁺.

Synthesis of 4-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoic acid (49): To a solution of tert-butyl 4-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (49a) (0.033 mmol) in dichloromethane (0.33 mL), trifluoroacetic acid (1.41 mmol) was added and the mixture was stirred at rt for 2 hours. The mixture was concentrated under reduced pressure. Reverse phase preparative HPLC employing water/MeCN as the eluent with 0.1% TFA modifier afforded 4-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoic acid (49). m/z): 809.57 [M+2H]²⁺. ¹H NMR (400 MHz, Methanol-d₄) δ 8.54 (s, 2H), 8.12 (d, J=2.7 Hz, 1H), 7.99-7.91 (m), 7.73-7.35 (m), 7.34-7.24 (m), 7.09 (d, J=7.9 Hz, 2H), 6.95 (d, J=2.7 Hz, 1H), 6.90 (d, J=10.1 Hz, 1H), 6.73 (s, 1H), 4.98 (t, J=7.6 Hz, 2H), 4.79-4.73 (m), 4.50-4.47 (m), 4.38-4.32 (m), 4.31-4.25 (m), 4.21-4.12 (m), 3.93-3.78 (m), 3.73 (s, 3H), 3.67 (s, 3H), 3.59-3.39 (m), 3.03-2.87 (m), 2.80-2.71 (m), 2.33-2.20 (m), 2.19-2.11 (m), 2.11-2.07 (m), 2.06-1.98 (m), 1.96 (s, 1H), 1.75 (s, 3H), 1.62 (s, 3H), 1.25-1.10 (m), 0.97 (s, 3H). ¹⁹F NMR (376 MHz, DMSO-d₆) δ −74.81 (s), −75.34 (s), −95.07 (d, J=20.2 Hz), −112.82 (s). ³¹P NMR (162 MHz, DMSO-d₆) δ −7.92 (s).

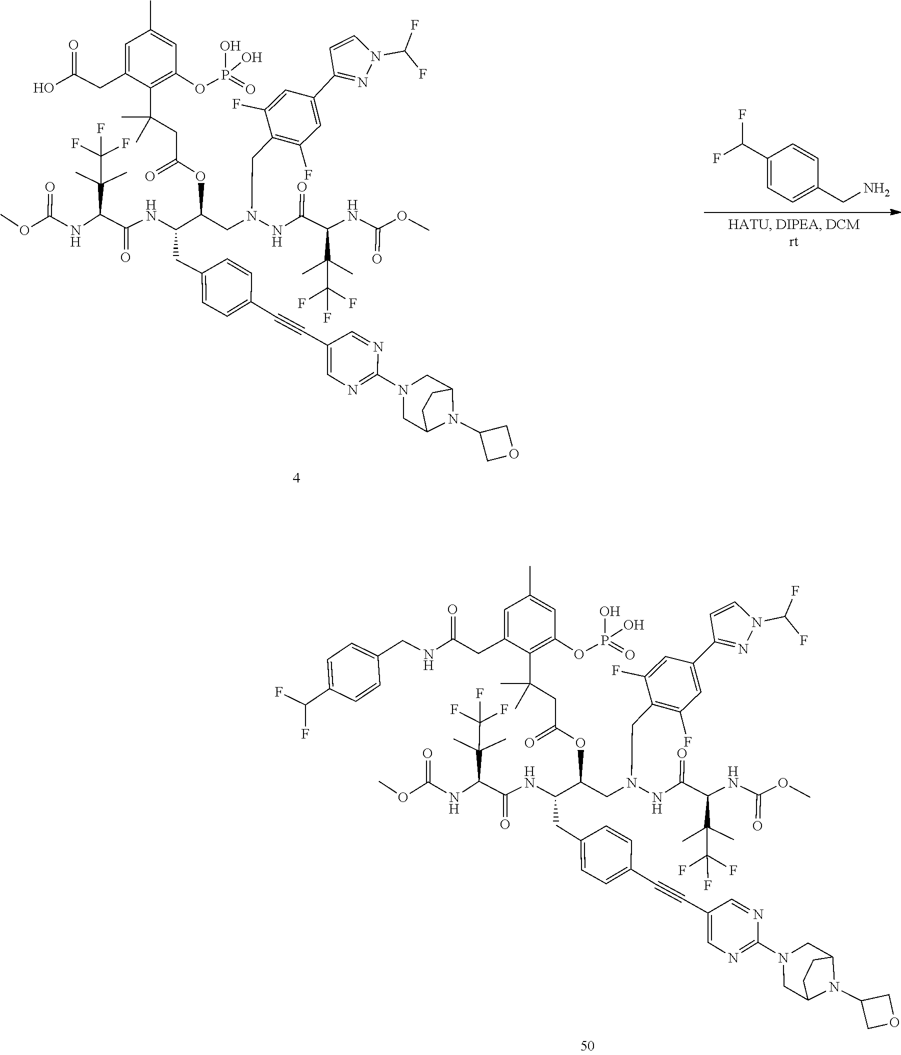 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; NCc1ccc(C(F)F)cc1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(C(F)F)cc2)cc(C)cc1OP(=O)(O)O)C(C)(C)C(F)(F)F]

### Example 50

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-(difluoromethyl)benzyl)amino)-2-oxoethyl)-4-methyl-6-(phosphonooxy)phenyl)-3-methylbutanoate (50): (5S,10S,11S,14S)-8-(4-(1-(difluoromethyl)-1H-pyrazol-3-yl)-2,6-difluorobenzyl)-11-(4-((2-(8-(oxetan-3-yl)-3, 8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-(difluoromethyl)benzyl)amino)-2-oxoethyl)-4-methyl-6-(phosphonooxy)phenyl)-3-methylbutanoate (50) was synthesized using the method described for Example 48, step 1, substituting (4-(difluoromethyl)phenyl)methanamine (1.4 eq) for methyl 2-amino-3-(1H-indol-3-yl)propanoate (2.0 eq). m/z): 1626.86 [M+H]⁺. ¹H NMR (400 MHz, Methanol-d₄) δ 8.57 (s, 2H), 8.12 (d, J=2.7 Hz, 1H), 7.73-7.42 (m), 7.41-7.30 (m), 7.08 (d, J=7.9 Hz, 2H), 6.95 (d, J=2.8 Hz, 1H), 6.94-6.82 (m), 6.70 (s, 1H), 6.67-6.61 (m), 5.00-4.93 (m), 4.81-4.76 (m), 4.56-4.37 (m), 4.37-4.25 (m), 4.22-4.08 (m), 3.92 (d, J=13.3 Hz, 1H), 3.84-3.76 (m), 3.73 (s, 3H), 3.67 (s, 3H), 3.63-3.52 (m), 3.06-2.94 (m), 2.88-2.74 (m), 2.30-2.20 (m), 2.19 (d, J=9.5 Hz, 1H), 2.04-1.97 (m), 1.97-1.90 (m), 1.74 (s, 3H), 1.59 (s, 3H), 1.34-1.28 (m), 1.25-1.14 (m), 0.99 (s, 3H). ¹⁹F NMR (376 MHz, Methanol-d₄) δ −77.10 (s), −77.76 (s), −96.94 (d, J=6.8 Hz), −112.07 (s), −114.38 (s). ³¹P NMR (162 MHz, Methanol-d₄) δ −6.63 (s).

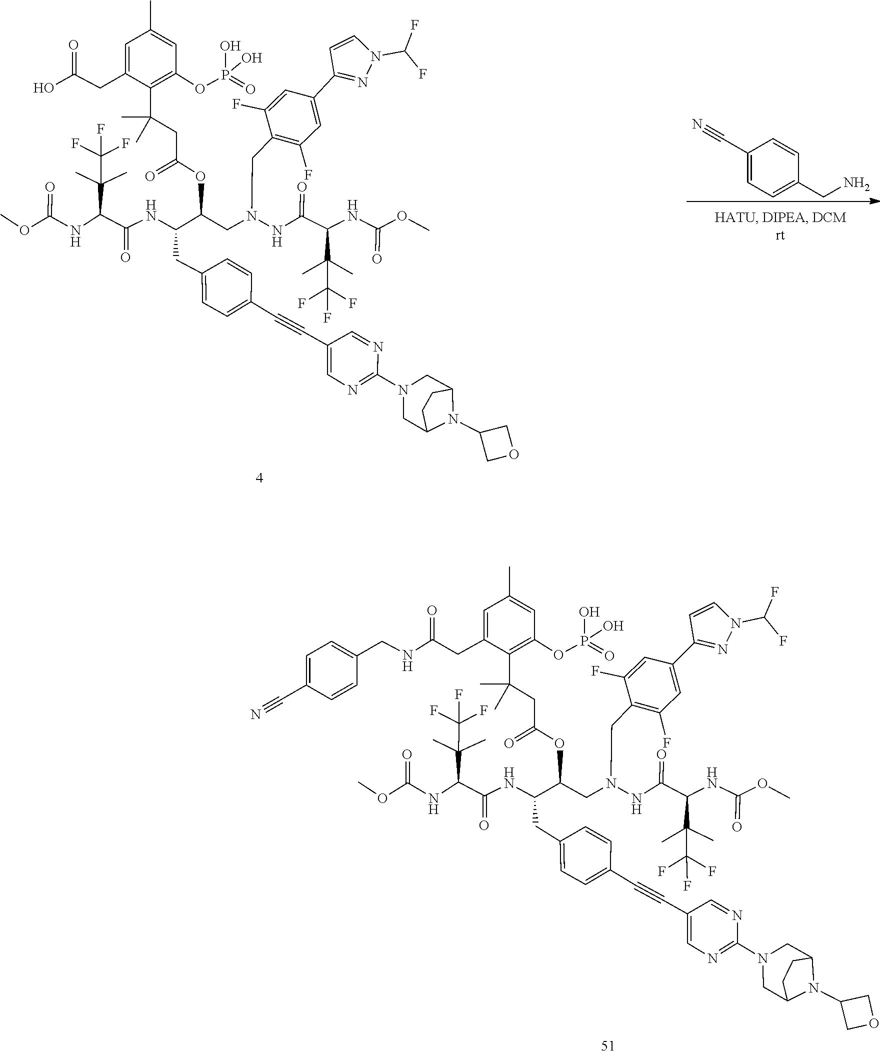 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; N#Cc1ccc(CN)cc1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(C#N)cc2)cc(C)cc1OP(=O)(O)O)C(C)(C)C(F)(F)F]

### Example 51

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-cyanobenzyl)amino)-2-oxoethyl)-4-methyl-6-(phosphonooxy)phenyl)-3-methylbutanoate (51): (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-cyanobenzyl)amino)-2-oxoethyl)-4-methyl-6-(phosphonooxy)phenyl)-3-methylbutanoate (51) was synthesized using the method described for Example 48, step 1, substituting 4-(aminomethyl)benzonitrile (1.4 eq) for methyl 2-amino-3-(1H-indol-3-yl)propanoate (2.0 eq). m/z): 1599.36 [M+H]⁺. ¹H NMR (400 MHz, Methanol-d₄) δ 8.55 (s, 2H), 8.12 (d, J=2.7 Hz, 1H), 7.72-7.52 (m), 7.48-7.38 (m), 7.36-7.28 (m), 7.07 (d, J=7.9 Hz, 2H), 6.98-6.88 (m), 6.71-6.64 (m), 4.98-4.93 (m), 4.81-4.74 (m), 4.60-4.39 (m), 4.37-4.25 (m), 4.22-4.06 (m), 3.91 (d, J=13.3 Hz, 1H), 3.82 (d, J=17.4 Hz, 1H), 3.73 (s, 3H), 3.68 (d, J=7.7 Hz, 3H), 3.57 (t, J=15.1 Hz, 2H), 3.08-2.92 (m), 2.90-2.71 (m), 2.32-2.18 (m), 2.16-2.07 (m), 2.04-1.96 (m), 1.75 (s, 3H), 1.60 (s, 3H), 1.24-1.12 (m), 0.99 (s, 3H). ¹⁹F NMR (376 MHz, Methanol-d₄) δ −77.10 (s), −77.73 (s), −96.92 (d, J=7.2 Hz), −114.41 (s). ³P NMR (162 MHz, Methanol-d₄) δ −6.81 (s).

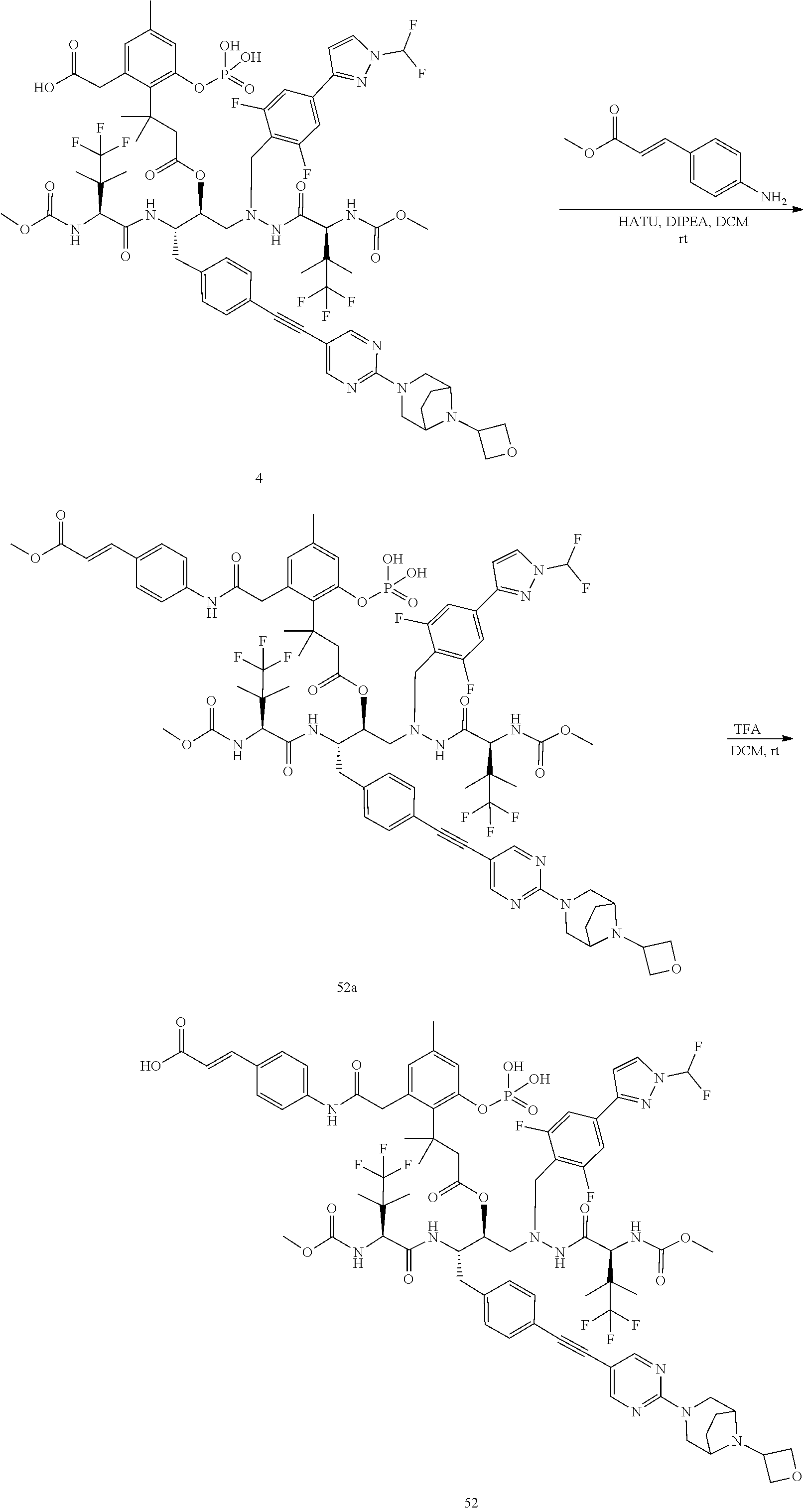

### Example 52

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-((E)-3-methoxy-3-oxoprop-1-en-1-yl)phenyl)amino)-2-oxoethyl)-4-methyl-6-(phosphonooxy)phenyl)-3-methylbutanoate (52a): (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-((E)-3-methoxy-3-oxoprop-1-en-1-yl)phenyl)amino)-2-oxoethyl)-4-methyl-6-(phosphonooxy)phenyl)-3-methylbutanoate (52a) was synthesized using the method described for Example 48, step 1, substituting methyl (E)-3-(4-aminophenyl)acrylate (2.5 eq) for methyl 2-amino-3-(1H-indol-3-yl)propanoate (2.0 eq). MS (m/z): 1644.17 [M+H]⁺.

Synthesis of (E)-3-(4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)phenyl)acrylic acid (52): (E)-3-(4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)phenyl)acrylic acid (52) was synthesized using the method described for Example 41, step 2, substituting (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((4-((E)-3-methoxy-3-oxoprop-1-en-1-yl)phenyl)amino)-2-oxoethyl)-4-methyl-6-(phosphonooxy)phenyl)-3-methylbutanoate (52a) for (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)tryptophan (48a). m/z): 1630.02 [M+H]⁺. ¹H NMR (400 MHz, Methanol-d4) δ 8.54 (s, 2H), 8.12 (d, J=7.1, 2.7 Hz, 1H), 7.73-7.47 (m), 7.46-7.39 (m), 7.35-7.28 (m), 7.09 (d, J=7.9 Hz, 2H), 6.97-6.90 (m), 6.74 (s, 1H), 6.35 (d, J=16.0 Hz, 1H), 5.51 (s, 2H), 5.03-4.93 (m), 4.80-4.74 (m), 4.44-4.32 (m), 4.32-4.21 (m), 4.21-4.09 (m), 3.93 (dd, J=46.7, 15.3 Hz, 2H), 3.73 (s, 3H), 3.67 (s, 3H), 3.65-3.46 (m), 3.37 (s, 1H), 3.05-2.76 (m), 2.39-2.12 (m), 2.12-1.99 (m), 1.82 (s, 3H), 1.67 (s, 3H), 1.24-1.15 (m), 1.01 (s, 3H). ¹⁹F NMR (376 MHz, Methanol-d4) δ −77.15 (s), −77.73 (s), −96.93 (d, J=11.7 Hz), −114.49 (s). ³¹P NMR (162 MHz, Methanol-d₄) δ −6.82 (s).

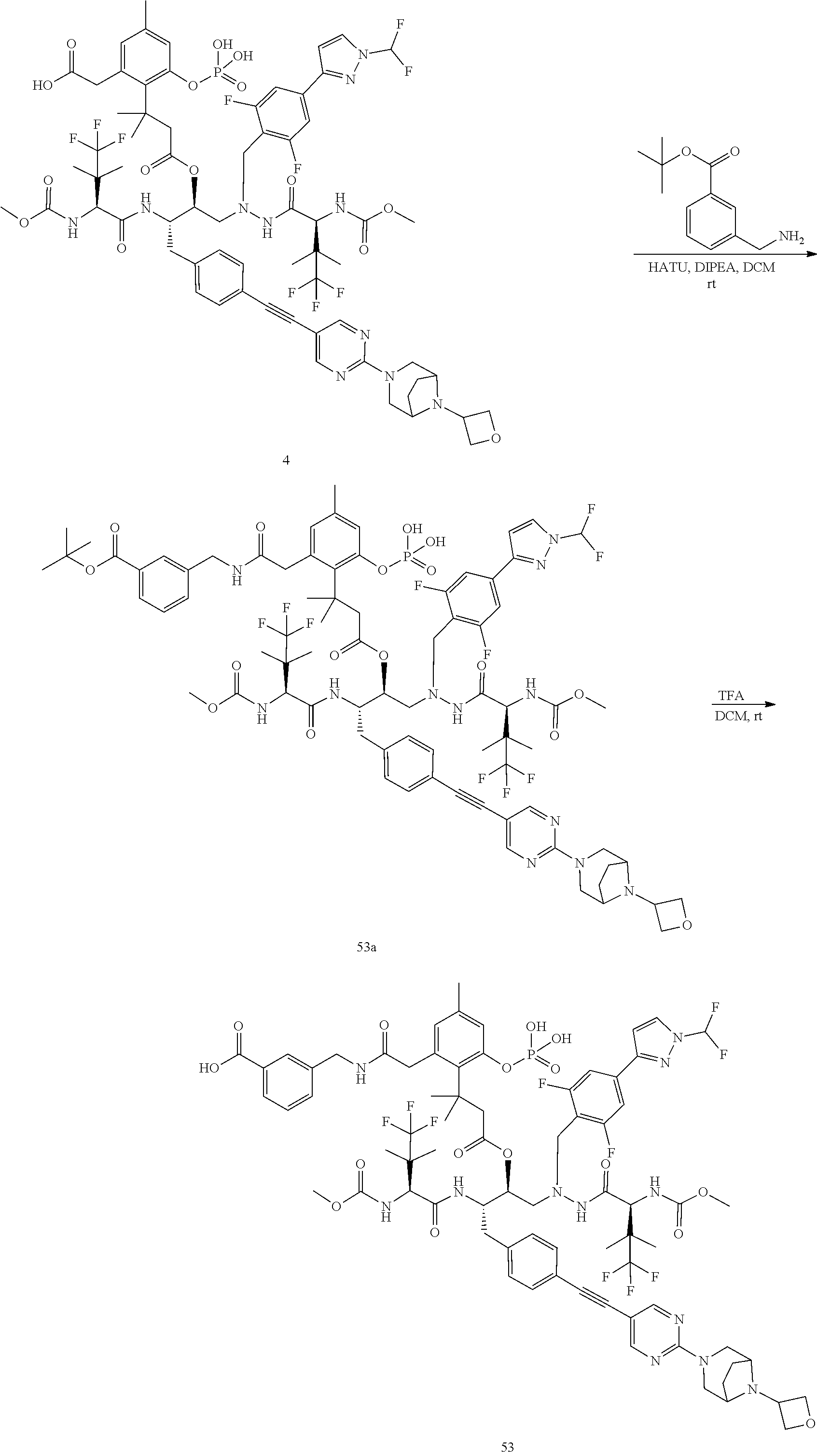

### Example 53

Synthesis of tert-butyl 3-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (53a): tert-butyl 3-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (53a) was synthesized using the method described for Example 49, step 1, substituting tert-butyl 3-(aminomethyl)benzoate for tert-butyl 4-(aminomethyl)benzoate. MS (m/z): 837.49 [M+2H]²⁺.

Synthesis of 3-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoic acid (53): 3-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoic acid (53) was synthesized using the method described for Example 49, step 2, substituting tert-butyl 3-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (53a) for tert-butyl 4-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)benzoate (49a). m/z): 1617.15 [M+H]⁺. ¹H NMR (400 MHz, Methanol-d₄) δ 8.55 (s, 2H), 8.12 (d, J=2.7 Hz, 1H), 7.99 (s, 1H), 7.89 (dt, J=7.7, 1.4 Hz, 1H), 7.73-7.36 (m), 7.33-7.26 (m), 7.08 (d, J=7.9 Hz, 2H), 6.95 (d, J=2.8 Hz, 1H), 6.90 (d, J=10.1 Hz, 1H), 6.72 (s, 1H), 5.01-4.93 (m), 4.80-4.74 (m), 4.51-4.44 (m), 4.39-4.25 (m), 4.22-4.10 (m), 3.90 (d, J=13.2 Hz, 1H), 3.80 (d, J=17.4 Hz, 1H), 3.73 (s, 3H), 3.67 (s, 3H), 3.64-3.40 (m), 3.03-2.84 (m), 2.83-2.69 (m), 2.32-2.19 (m), 2.18-2.09 (m), 2.07-1.97 (m), 1.74 (s, 3H), 1.61 (s, 3H), 1.24-1.13 (m), 0.97 (s, 3H). ¹⁹F NMR (376 MHz, Methanol-d₄) δ −77.17 (s), −77.79 (s), −96.93 (d, J=7.2 Hz), −114.38 (s). ³¹P NMR (162 MHz, Methanol-d4) δ −6.77 (s).

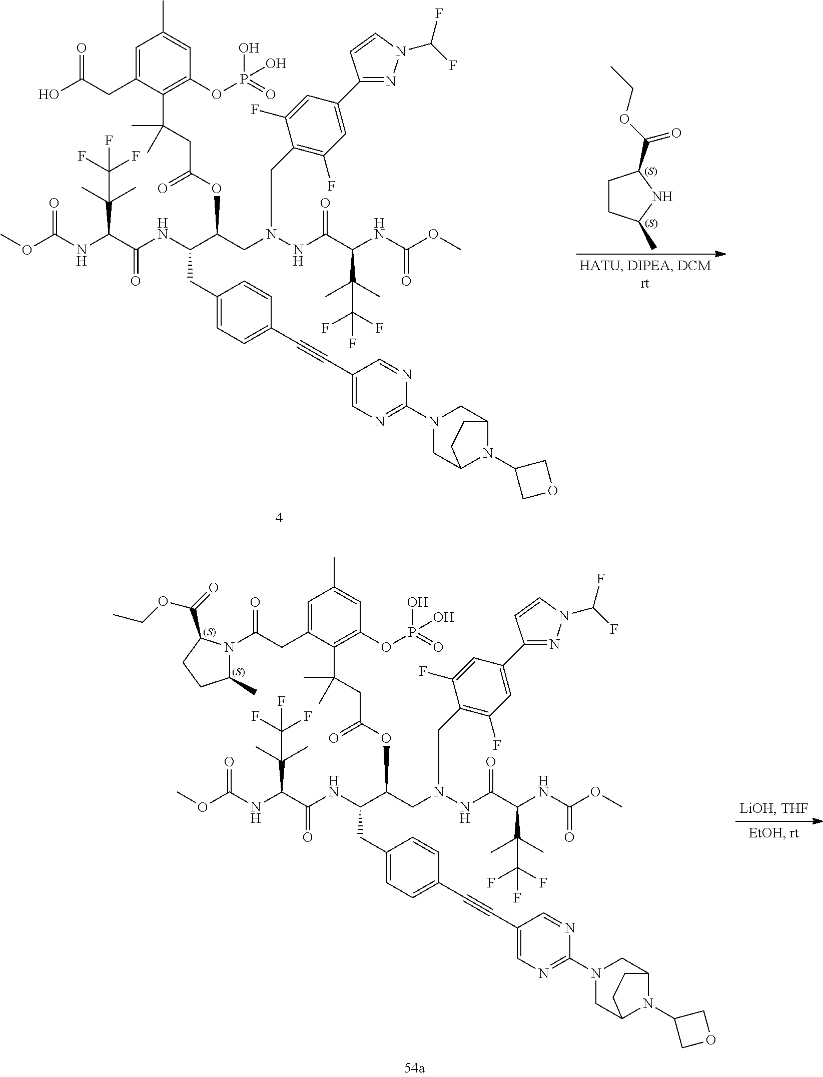

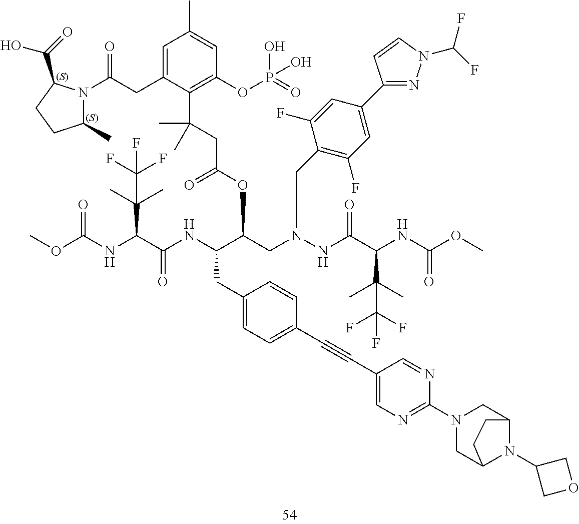

### Example 54

Synthesis of ethyl (2S,5S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-5-methylpyrrolidine-2-carboxylate (54a): To a solution of 2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetic acid (4) (0.101 mmol) and ethyl (2S,5S)-5-methylpyrrolidine-2-carboxylate (0.152 mmol) in dichloromethane (1 mL), N, N, N-diisopropylethylamine (DIPEA) (0.07 mL) and HATU (0.111 mmol) were added in a sequential manner and the mixture was stirred at rt for 1 hour. The mixture was quenched by addition of 0.25 mL trifluoroacetic acid (TFA) and concentrated under reduced pressure. Reverse phase preparative HPLC employing water/MeCN as the eluent with 0.1% TFA modifier afforded ethyl (2S,5S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-5-methylpyrrolidine-2-carboxylate (54a). MS (m/z): 1623.96 [M+H]⁺.

Synthesis of (2S,5S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-5-methylpyrrolidine-2-carboxylic acid (54): To a solution of ethyl (2S,5S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-5-methylpyrrolidine-2-carboxylate (54a) (0.076 mmol) in THE (0.76 mL) and ethanol (0.38 mL), 1 M lithium hydroxide solution (0.76 mL) was added in a drop wise manner and the mixture was stirred at rt for 1.5 hours. The mixture was quenched by addition of 0.25 mL TFA and concentrated under reduced pressure. Reverse phase preparative HPLC employing water/MeCN as the eluent with 0.1% TFA modifier afforded (2S,5S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-5-methylpyrrolidine-2-carboxylic acid (54). m/z): 798.51 [M+2H]²⁺. ¹H NMR (400 MHz, Methanol-d₄) δ 8.56-8.53 (m), 8.14-8.10 (m), 7.73-7.39 (m), 7.33-7.28 (m), 7.25 (s, 1H), 7.14-7.08 (m), 6.98-6.93 (m), 6.69-6.54 (m), 4.98 (t, J=7.6 Hz, 2H), 4.77-4.73 (m), 4.48-4.40 (m), 4.33-4.25 (m), 4.25-4.10 (m), 3.98-3.91 (m), 3.75-3.72 (m), 3.70-3.65 (m), 3.57-3.45 (m), 3.04-2.93 (m), 2.84-2.71 (m), 2.40-2.29 (m), 2.28-2.20 (m), 2.16-1.98 (m), 1.85-1.77 (m), 1.73 (s, 3H), 1.63 (s, 3H), 1.36 (dd, J=6.3, 1.7 Hz, 3H), 1.25-1.10 (m), 1.05 (s, 2H), 0.96 (s, 1H). ¹⁹F NMR (376 MHz, DMSO-d₆) δ −74.81 (s), −75.36 (s), −95.06 (d, J=30.2 Hz), −112.76 (s). ³¹P NMR (162 MHz, Methanol-d4) δ −7.07 (s).

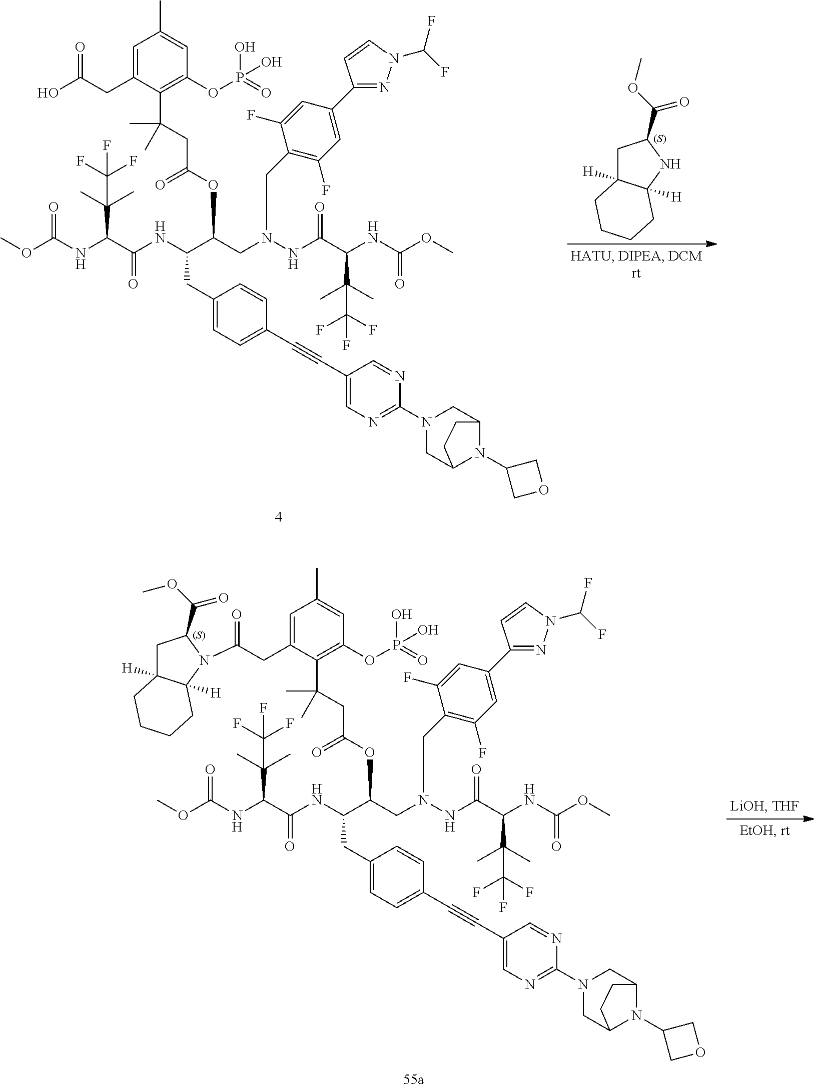

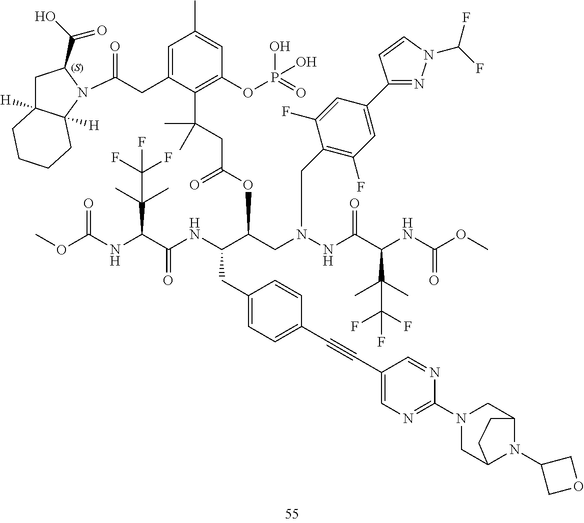

Synthesis of methyl (2S,3aS,7aS)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)octahydro-1H-indole-2-carboxylate (55a): Methyl (2S,3aS,7aS)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)octahydro-1H-indole-2-carboxylate (55a) was synthesized using the method described for Example 48, step 1, substituting methyl (2S,3aS,7aS)-octahydro-1H-indole-2-carboxylate (1.5 eq) for methyl 2-amino-3-(1H-indol-3-yl)propanoate (2.0 eq), DIPEA (4.0 eq) for DIPEA (6.0 eq) and HATU (1.1 eq) for HATU (1.5 eq). MS (m/z): 1649.21 [M+H]⁺.

Synthesis of (2S,3aS,7aS)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)octahydro-1H-indole-2-carboxylic acid (55): (2S,3aS,7aS)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)octahydro-1H-indole-2-carboxylic acid (55) was synthesized using the method described for Example 48, step 2, substituting methyl (2S,3aS,7aS)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)octahydro-1H-indole-2-carboxylate (55a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 818.54 [M+2H]²⁺. ¹H NMR (400 MHz, Methanol-d4) δ 8.55 (s, 2H), 8.12 (d, J=2.7 Hz, 1H), 7.73-7.39 (m), 7.30 (d, J=7.9 Hz, 2H), 7.25 (s, 1H), 7.11 (d, J=7.9 Hz, 2H), 6.99-6.95 (m), 6.66 (s, 1H), 4.98 (t, J=7.6 Hz, 2H), 4.46-4.38 (m), 4.36-4.28 (m), 4.25-4.19 (m), 4.19-4.14 (m), 3.98-3.91 (m), 3.88-3.80 (m), 3.74 (s, 3H), 3.68 (s, 3H), 3.57-3.48 (m), 3.44 (d, J=15.5 Hz, 1H), 3.05-2.93 (m), 2.87-2.80 (m), 2.48-2.30 (m), 2.29-2.18 (m), 2.13-2.05 (m), 2.04-1.97 (m), 1.77-1.70 (m), 1.67-1.60 (m), 1.25-1.15 (m), 1.08-0.91 (m). ¹⁹F NMR (376 MHz, Methanol-d₄) δ −77.14 (s), −77.70 (s), −96.94 (d, J=6.7 Hz), −114.47 (s). ³¹P NMR (162 MHz, Methanol-d₄) δ −7.07 (s).

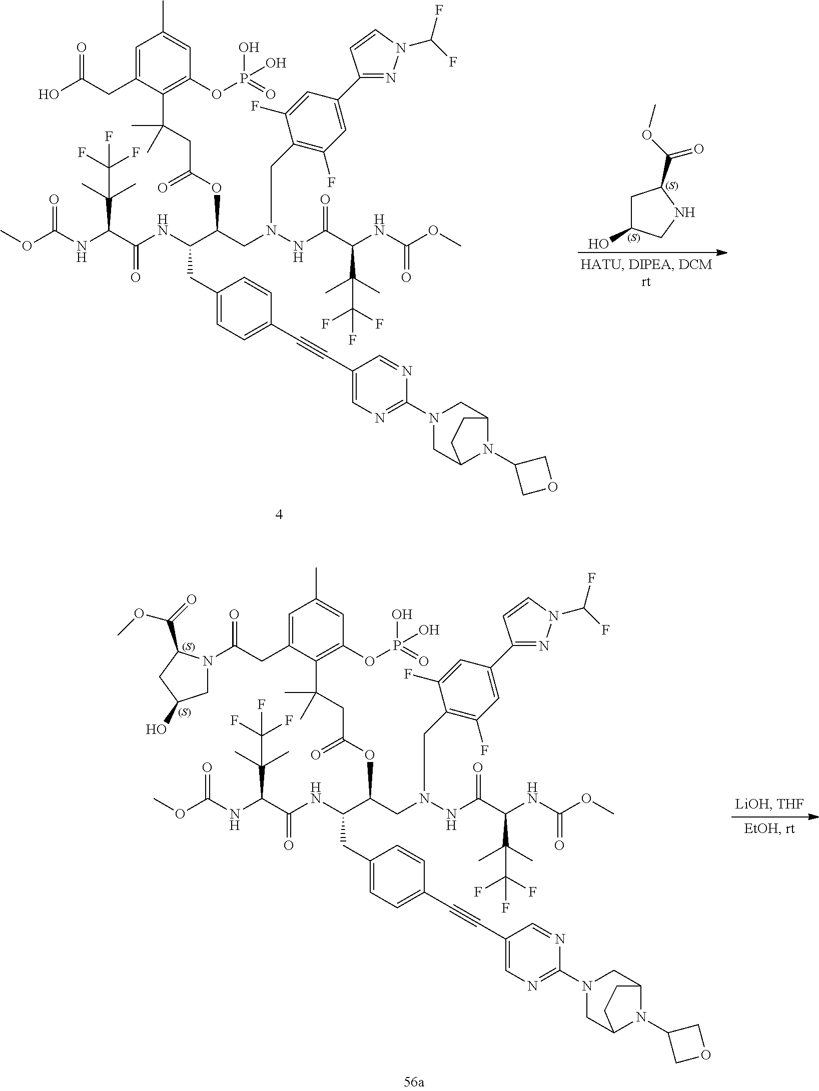

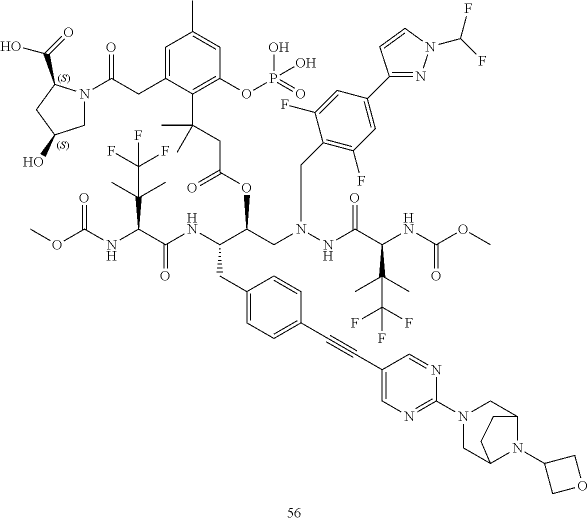

### Example 56

Synthesis of methyl (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylate (56a): Methyl (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylate (56a) was synthesized using the method described for Example 48, step 1, substituting methyl (2S,4S)-4-hydroxypyrrolidine-2-carboxylate hydrochloride (1.5 eq) for methyl 2-amino-3-(1H-indol-3-yl)propanoate (2.0 eq) and HATU (1.1 eq) for HATU (1.2 eq). MS (m/z): 806.59 [M+2H]²⁺.

Synthesis of (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylic acid (56): (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylic acid (56) was synthesized using the method described for Example 48, step 2 substituting methyl (2S,4S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylate (56a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 799.50 [M+2H]²⁺. ¹H NMR (400 MHz, Methanol-d₄) δ 8.59 (s, 2H), 8.12 (d, J=2.8 Hz, 1H), 7.73-7.42 (m), 7.42-7.34 (m), 7.29 (s, 1H), 7.12 (d, J=8.3 Hz, 2H), 7.01-6.90 (m), 5.11-5.01 (m), 4.60-4.49 (m), 4.46 (s, 1H), 4.36-4.27 (m), 4.27-4.17 (m), 4.08 (s, 1H), 4.02-3.92 (m), 3.88-3.80 (m), 3.72 (s, 3H), 3.68 (s, 3H), 3.56-3.45 (m), 3.04-2.91 (m), 2.87-2.76 (m), 2.49-2.36 (m), 2.49-2.36 (m), 2.29-2.20 (m), 2.11 (d, J=13.3 Hz, 1H), 2.07-1.92 (m), 1.85-1.72 (m), 1.68-1.56 (m), 1.25-1.15 (m), 1.11-0.99 (m). ¹⁹F NMR (376 MHz, Methanol-d₄) δ −77.59 (s), −77.69 (s), −96.94 (d, J=5.3 Hz), −114.36 (s). ³¹P NMR (162 MHz, Methanol-d4) δ −7.09 (s).

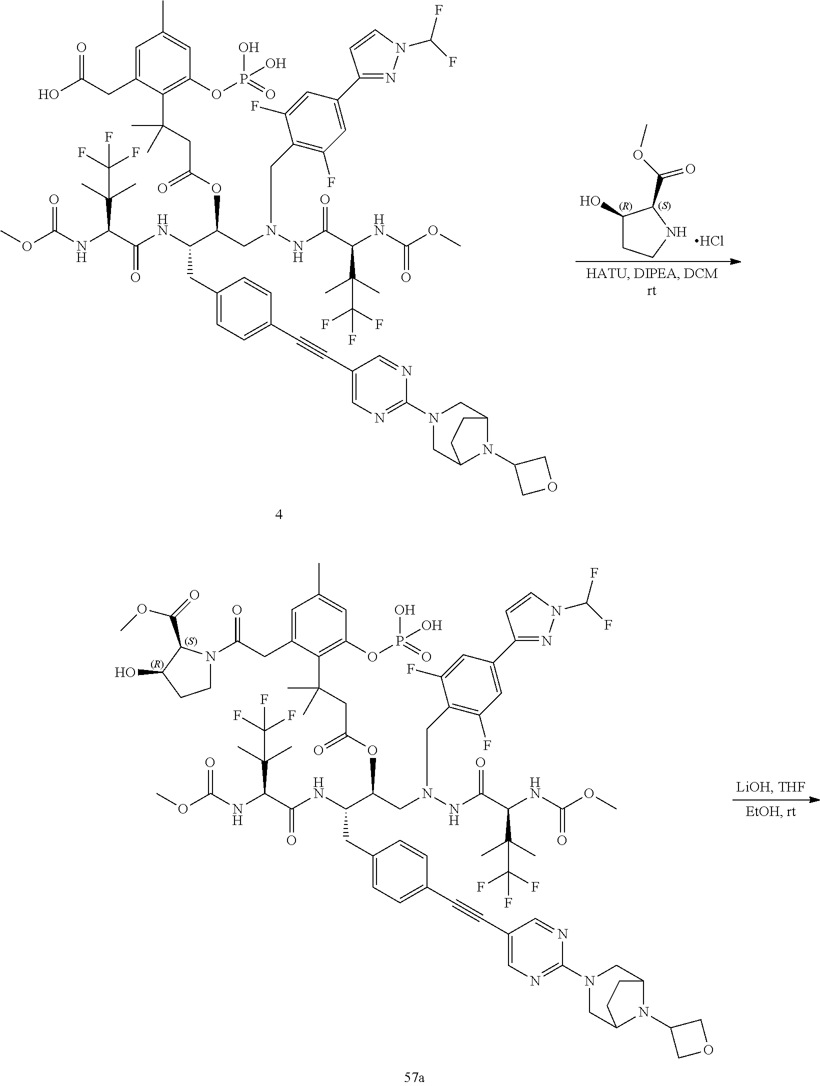

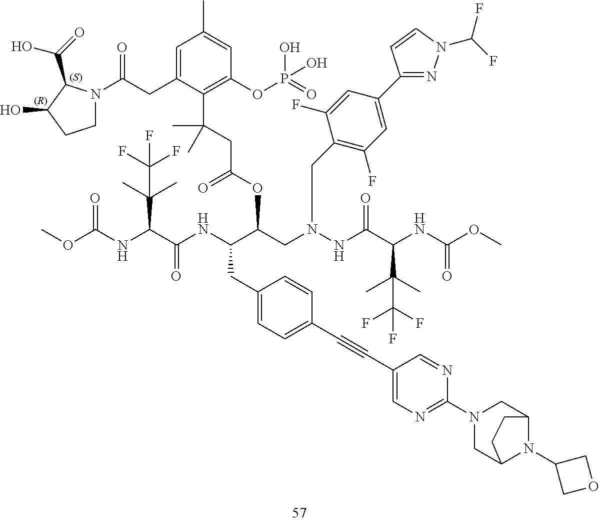

### Example 57

Synthesis of methyl (2S,3R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-3-hydroxypyrrolidine-2-carboxylate (57a): Methyl (2S,3R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-3-hydroxypyrrolidine-2-carboxylate (57a) was synthesized using the method described for Example 48, step 1, substituting methyl (2S,3R)-3-hydroxypyrrolidine-2-carboxylate hydrochloride (1.5 eq) for methyl 2-amino-3-(1H-indol-3-yl)propanoate (2.0 eq) and HATU (1.1 eq) for HATU (1.5 eq). m/z): 806.48 [M+2H]²⁺.

Synthesis of (2S,3R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-3-hydroxypyrrolidine-2-carboxylic acid (57): (2S,3R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-3-hydroxypyrrolidine-2-carboxylic acid (57) was synthesized using the method described for Example 48, step 2 substituting methyl (2S,3R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-3-hydroxypyrrolidine-2-carboxylate (57a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 799.51 [M+2H]²⁺. ¹H NMR (400 MHz, Methanol-d₄) δ 8.57 (s, 2H), 8.12 (d, J=2.7 Hz, 1H), 7.73-7.39 (m), 7.36 (d, J=7.8 Hz, 2H), 7.31-7.24 (m), 7.14-7.05 (m), 6.98-6.93 (m), 6.60 (s, 1H), 5.09-5.02 (m), 4.85-4.76 (m), 4.68-4.47 (m), 4.39-4.29 (m), 4.29-4.15 (m), 4.13-4.05 (m), 4.02-3.84 (m), 3.72 (s, 3H), 3.69 (s, 3H), 3.05-2.93 (m), 2.84-2.73 (m), 2.67 (d, J=15.4 Hz, 1H), 2.29-2.20 (m), 2.20-2.10 (m), 2.01-1.93 (m), 1.88-1.81 (m), 1.80-1.69 (m), 1.62 (s, 3H), 1.26-1.16 (m), 1.08 (s, 3H). ¹⁹F NMR (376 MHz, DMSO-d₆) δ −74.77 (s), −75.34 (s), −95.07 (d, J=18.1 Hz), −112.82 (s). ³¹P NMR (162 MHz, Methanol-d₄) δ −7.17.

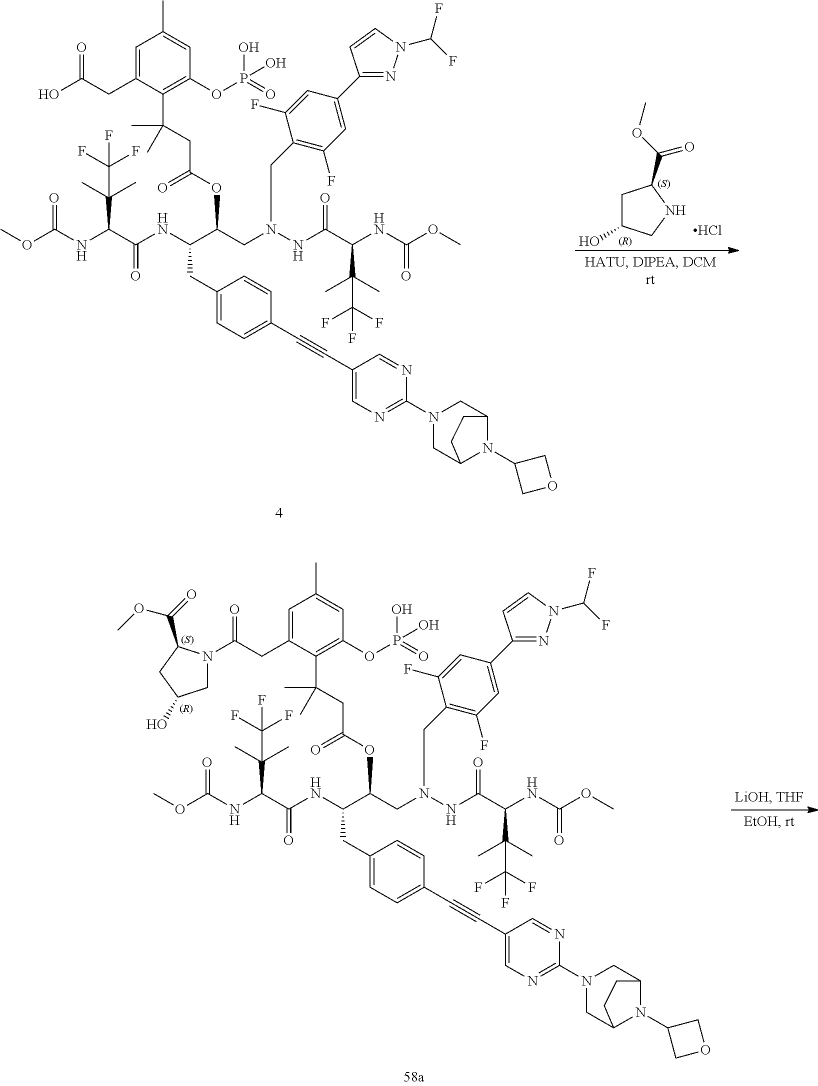

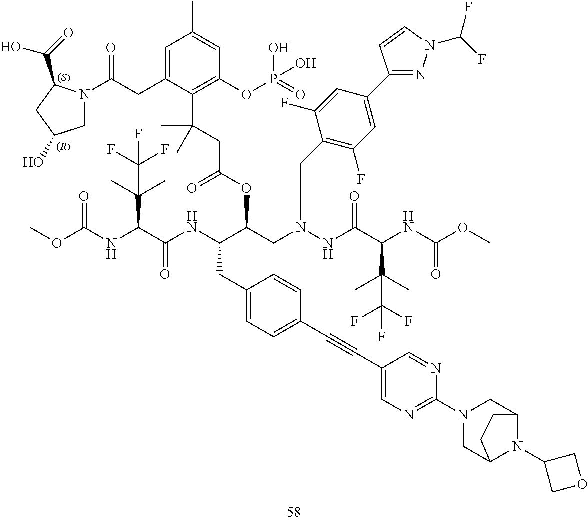

### Example 58

Synthesis of methyl (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylate (58a): Methyl (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylate (58a) was synthesized using the method described for Example 48, step 1 substituting methyl (2S,4R)-4-hydroxypyrrolidine-2-carboxylate hydrochloride (1.25 eq) for methyl 2-amino-3-(1H-indol-3-yl)propanoate (2.0 eq) and HATU (1.1 eq) for HATU (1.5 eq). MS (m/z): 806.60 [M+2H]²⁺.

Synthesis of (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylic acid (58): (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylic acid (58) was synthesized using the method described for Example 41, step 2 substituting methyl (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-hydroxypyrrolidine-2-carboxylate (58a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 799.61 [M+2H]²⁺. ¹H NMR (400 MHz, Methanol-d₄) δ 8.55 (s, 2H), 8.12 (d, J=2.8 Hz, 1H), 7.72-7.37 (m), 7.33-7.28 (m), 7.27-7.21 (m), 7.10 (d, J=7.8 Hz, 2H), 6.98-6.91 (m), 6.71 (s, 1H), 4.98 (t, J=7.6 Hz, 2H), 4.55 (t, J=8.0 Hz, 1H), 4.50-4.41 (m), 4.34-4.26 (m), 4.23 (s, 1H), 4.22-4.19 (m), 4.02-3.87 (m), 3.73 (s, 3H), 3.68 (s, 3H), 3.65-3.60 (m), 3.58-3.46 (m), 3.01-2.91 (m), 2.85-2.73 (m), 2.40-2.20 (m), 2.15-2.10 (m), 2.10-2.05 (m), 2.04-1.98 (m), 1.80-1.70 (m), 1.63 (s, 3H), 1.26-1.12 (m), 1.08-0.96 (m). ¹⁹F NMR (376 MHz, DMSO-d₆) δ −74.79 (s), −75.32 (s), −95.07 (d, J=19.6 Hz), −112.78 (s). ³¹P NMR (162 MHz, DMSO-d₆) δ −8.01 (s).

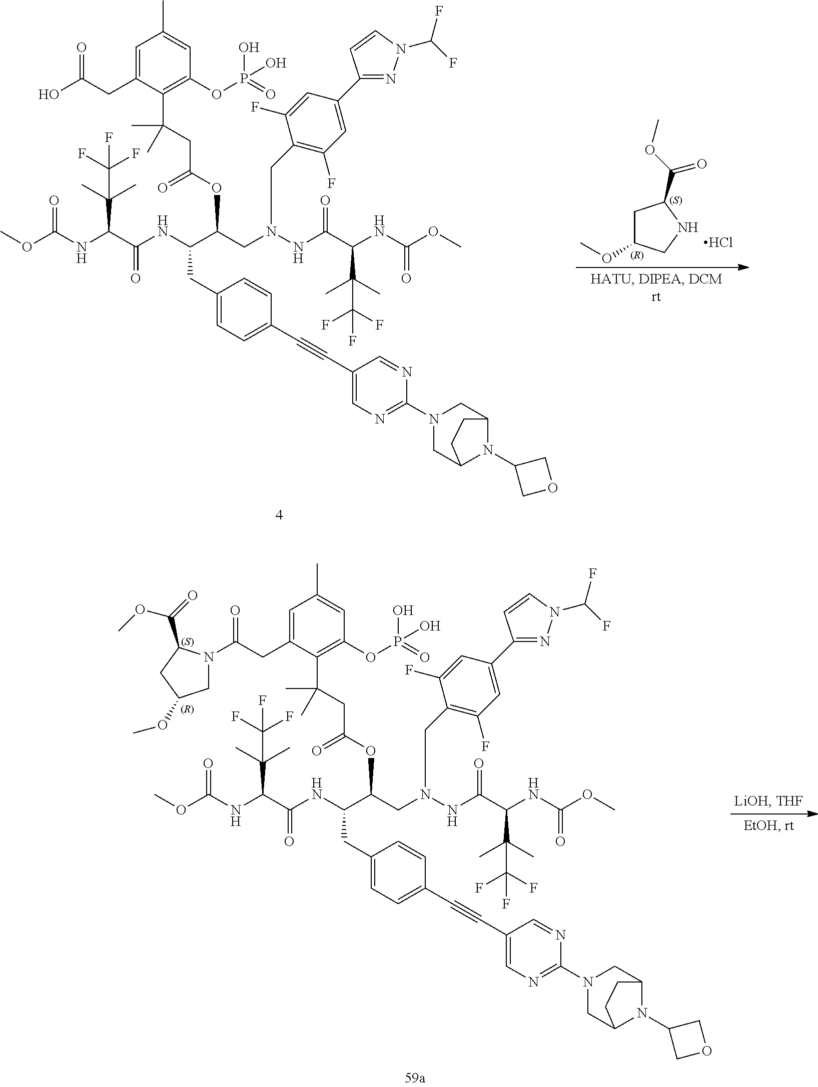

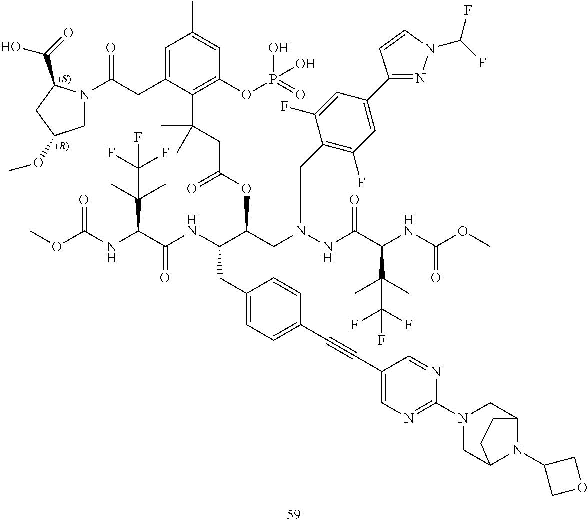

### Example 59

Synthesis of methyl (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methoxypyrrolidine-2-carboxylate (59a): Methyl (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methoxypyrrolidine-2-carboxylate (59a) was synthesized using the method described for Example 48, step 1, substituting methyl (2S,4R)-4-methoxypyrrolidine-2-carboxylate hydrochloride (1.25 eq) for methyl 2-amino-3-(1H-indol-3-yl)propanoate (2.0 eq) and HATU (1.1 eq) for HATU (1.5 eq). MS (m/z): 813.53 [M+2H]²⁺.

Synthesis of (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methoxypyrrolidine-2-carboxylic acid (59): (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methoxypyrrolidine-2-carboxylic acid (59) was synthesized using the method described for Example 41, step 2 substituting methyl (2S,4R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methoxypyrrolidine-2-carboxylate (59a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 806.60 [M+2H]²⁺. ¹H NMR (400 MHz, Methanol-d₄) δ 8.65-8.53 (m), 8.12 (d, J=2.8 Hz, 1H), 7.73-7.42 (m), 7.42-7.35 (m), 7.32-7.25 (m), 7.15-7.06 (m), 6.99-6.90 (m), 6.58-6.44 (m), 5.11-5.05 (m), 4.61-4.49 (m), 4.45 (t, J=8.0 Hz, 1H), 4.38-4.29 (m), 4.29-4.23 (m), 4.23-4.18 (m), 4.11-4.03 (m), 4.02-3.93 (m), 3.86-3.80 (m), 3.72 (s, 3H), 3.68 (s), 3.64-3.58 (m), 3.06-2.94 (m), 2.86-2.76 (m), 2.66 (d, J=15.3 Hz, 1H), 2.46-2.36 (m), 2.30-2.18 (m), 2.13-2.07 (m), 2.05 (s, 1H), 1.99 (d, J=10.2 Hz, 2H), 1.86-1.71 (m), 1.63 (s, 3H), 1.30-1.15 (m), 1.13-1.06 (m). ¹⁹F NMR (376 MHz, DMSO-d₆) δ −74.84 (s), −75.35 (s), −95.08 (d, J=19.8 Hz), −112.83 (s). ³¹P NMR (162 MHz, DMSO-d₆) δ −7.95 (s).

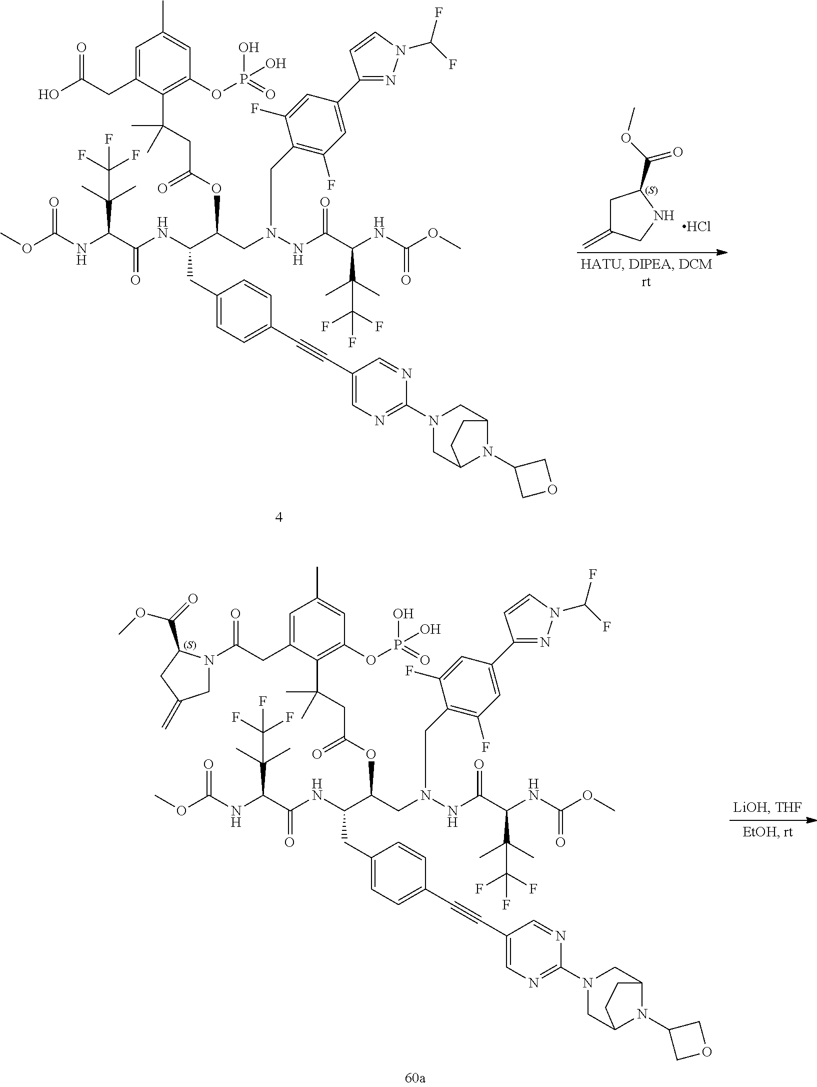

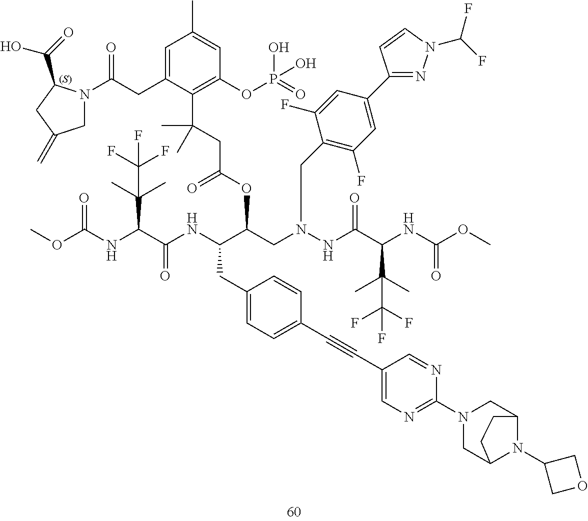

### Example 60

Synthesis of methyl (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methylenepyrrolidine-2-carboxylate (60a): Methyl (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methylenepyrrolidine-2-carboxylate (60a) was synthesized using the method described for Example 48, step 1, substituting methyl (S)-4-methylenepyrrolidine-2-carboxylate hydrochloride (1.25 eq) for methyl 2-amino-3-(1H-indol-3-yl)propanoate (2.0 eq) and HATU (1.1 eq) for HATU (1.5 eq). MS (m/z): 804.56 [M+2H]^{2+.}

Synthesis of (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methylenepyrrolidine-2-carboxylic acid (60): (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methylenepyrrolidine-2-carboxylic acid (60) was synthesized using the method described for Example 48, step 2 substituting methyl (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4-methylenepyrrolidine-2-carboxylate (60a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 797.62 [M+2H]²⁺. ¹H NMR (400 MHz, Methanol-d₄) δ 8.54 (s), 8.12 (d, J=2.7 Hz, 1H), 7.74-7.38 (m), 7.34-7.28 (m), 7.28-7.24 (m), 7.14-7.07 (m), 6.99-6.93 (m), 6.75-6.67 (m), 5.14-5.05 (m), 4.98 (t, J=7.6 Hz, 2H), 4.81-4.69 (m), 4.38-4.27 (m), 4.27-4.14 (m), 3.98-3.85 (m), 3.74 (s, 3H), 3.68 (s, 3H), 3.56-3.46 (m), 3.17-3.01 (m), 2.99-2.89 (m), 2.88-2.66 (m), 2.40-2.30 (m), 2.28-2.19 (m), 2.10-2.05 (m), 2.05-1.98 (m), 1.79-1.70 (m), 1.63 (s, 3H), 1.25-1.12 (m), 1.08-0.99 (m). ¹⁹F NMR (376 MHz, DMSO-d₆) δ −77.96 (s), −78.56 (s), −97.76 (d, J=7.6 Hz), −115.24 (s). P NMR (162 MHz, DMSO-d₆) δ −7.94 (s).

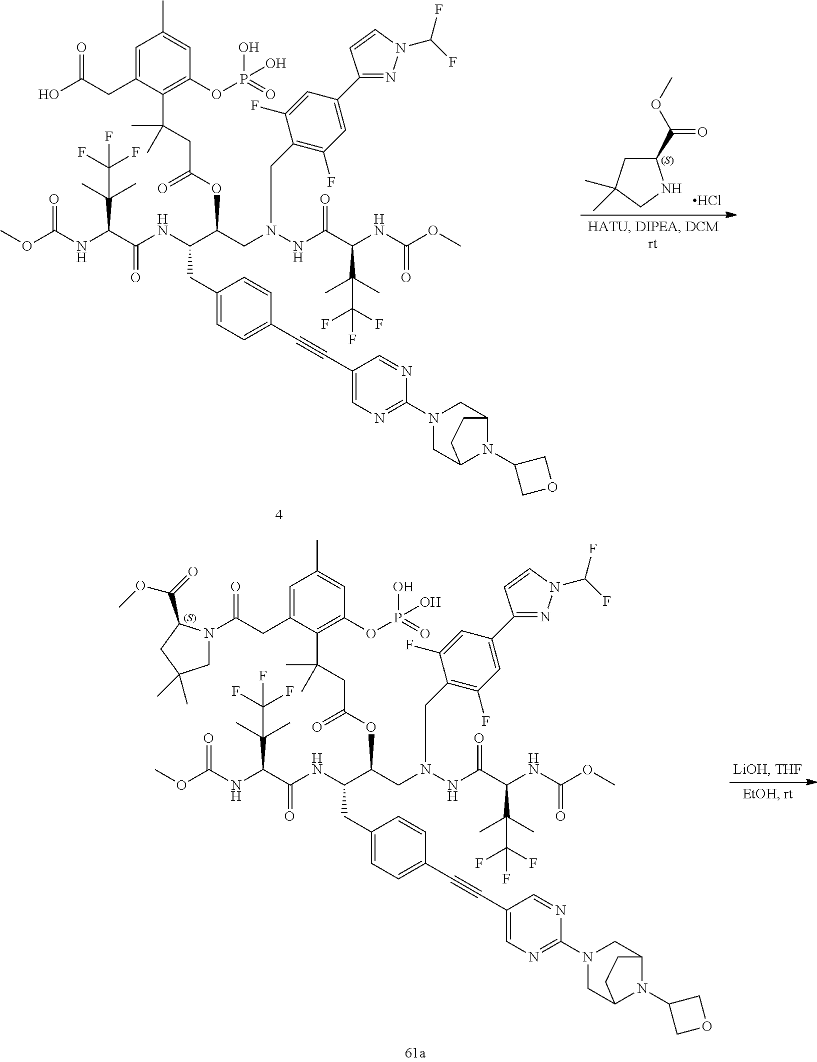

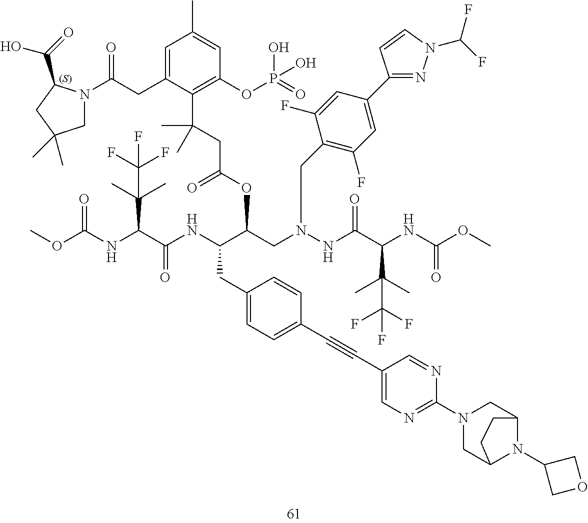

### Example 61

Synthesis of methyl (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4,4-dimethylpyrrolidine-2-carboxylate (61a): Methyl (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4,4-dimethylpyrrolidine-2-carboxylate (61a) was synthesized using the method described for Example 48, step 1, substituting methyl (S)-4,4-dimethylpyrrolidine-2-carboxylate hydrochloride (1.25 eq) for methyl 2-amino-3-(1H-indol-3-yl)propanoate (2.0 eq) and HATU (1.1 eq) for HATU (1.5 eq). MS (m/z): 812.64 [M+2H]²⁺.

Synthesis of (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4,4-dimethylpyrrolidine-2-carboxylic acid (61): (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4,4-dimethylpyrrolidine-2-carboxylic acid (61) was synthesized using the method described for Example 48, step 2 substituting methyl (2S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)-4,4-dimethylpyrrolidine-2-carboxylate (61a) for (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-((3-(1H-indol-3-yl)-1-methoxy-1-oxopropan-2-yl)amino)-2-oxoethyl)-4-methyl-6-(phosphonooxy)phenyl)-3-methylbutanoate (48a). m/z): 805.59 [M+2H]²⁺. ¹H NMR (400 MHz, Methanol-d₄) δ 8.55 (s), 8.12 (d, J=2.7 Hz, 1H), 7.72-7.37 (m), 7.30 (d, J=7.8 Hz, 2H), 7.28-7.23 (m), 7.12-7.06 (m), 6.99-6.94 (m), 6.69 (s, 1H), 4.98 (t, J=7.6 Hz, 2H), 4.76-4.71 (m), 4.52-4.27 (m), 4.26-4.11 (m), 4.01-3.90 (m), 3.83-3.76 (m), 3.73 (s, 3H), 3.68 (s, 3H), 3.56-3.44 (m), 3.05-2.93 (m), 2.80-2.68 (m), 2.36-2.20 (m), 2.20-2.10 (m), 2.09-1.93 (m), 1.84-1.69 (m), 1.63 (s, 3H), 1.26-1.12 (m), 1.18-1.03 (m). ¹⁹F NMR (376 MHz, DMSO-d₆) δ −74.82 (s), −75.36 (s), −95.08 (d, J=18.4 Hz), −112.79 (s). ³P NMR (162 MHz, DMSO-d₆) δ −8.03 (s).

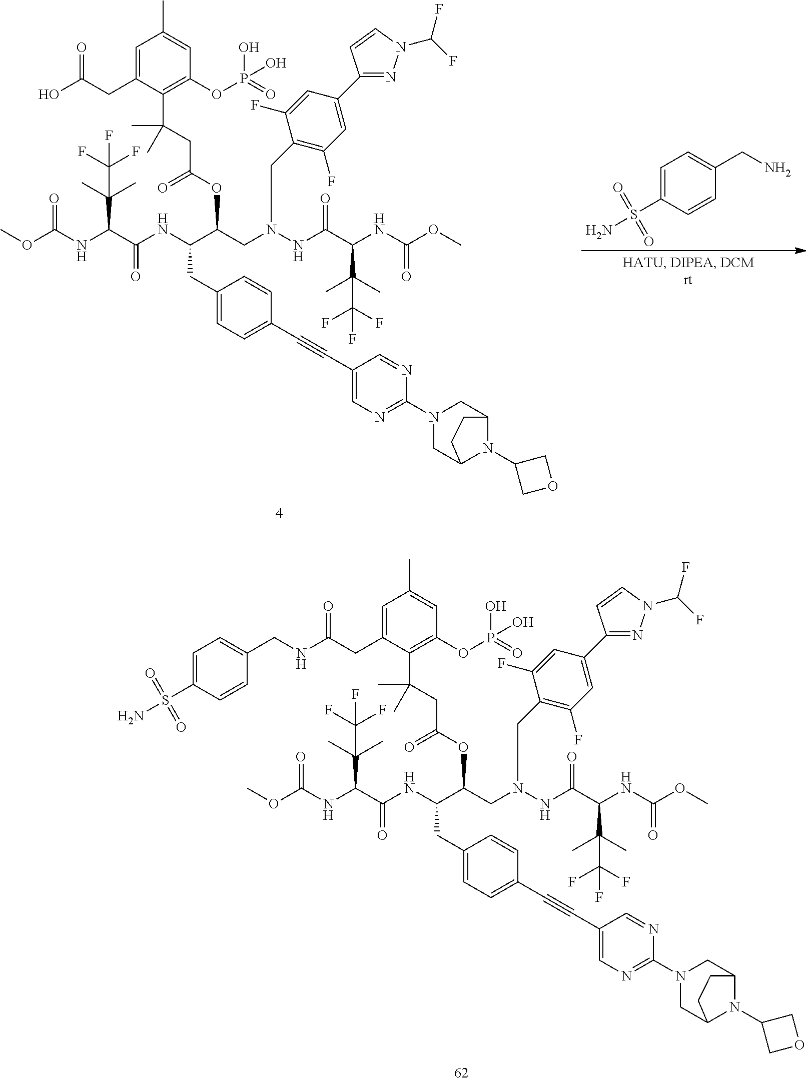 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; NCc1ccc(S(N)(=O)=O)cc1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCc2ccc(S(N)(=O)=O)cc2)cc(C)cc1OP(=O)(O)O)C(C)(C)C(F)(F)F]

### Example 62

Synthesis of (5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-oxo-2-((4-sulfamoylbenzoyl)amino)ethyl)-6-(phosphonooxy)phenyl)butanoate (62): (5S,10S,11S,14S)-8-(4-(1-(difluoromethyl)-1H-pyrazol-3-yl)-2,6-difluorobenzyl)-11-(4-((2-(8-(oxetan-3-yl)-3, 8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-oxo-2-((4-sulfamoylbenzoyl)amino)ethyl)-6-(phosphonooxy)phenyl)butanoate (62) was synthesized using the method described for Example 48, step 1, substituting 4-(aminomethyl)benzenesulfonamide (1.3 eq) for methyl 2-amino-3-(1H-indol-3-yl)propanoate (2.0 eq) and HATU (1.3 eq) for HATU (1.5 eq). m/z): 1653.65 [M+H]⁺. ¹H NMR (400 MHz, Methanol-d₄) δ 8.55 (s, 2H), 8.12 (d, J=2.7 Hz, 1H), 7.86-7.82 (m), 7.73-7.38 (m), 7.33-7.29 (m), 7.09 (d, J=7.9 Hz, 2H), 6.95 (d, J=2.8 Hz, 1H), 6.91 (d, J=10.2 Hz, 1H), 6.70 (s, 1H), 4.98 (t, J=7.6 Hz, 2H), 4.79-4.75 (m), 4.55-4.40 (m), 4.40-4.25 (m), 4.22-4.12 (m), 3.93 (d, J=13.2 Hz, 1H), 3.79 (d, J=17.2 Hz, 1H), 3.73 (s, 3H), 3.67 (s, 3H), 3.60-3.52 (m), 2.94-2.82 (m), 2.27-2.19 (m), 2.10-1.98 (m), 1.74 (s, 3H), 1.61 (s, 3H), 1.25-1.13 (m), 0.98 (s, 3H). ¹⁹F NMR (376 MHz, DMSO-d₆) δ −74.82 (s), −75.34 (s), −95.07 (d, J=21.1 Hz), −112.81 (s). P NMR (162 MHz, DMSO-d₆) δ −7.95 (s).

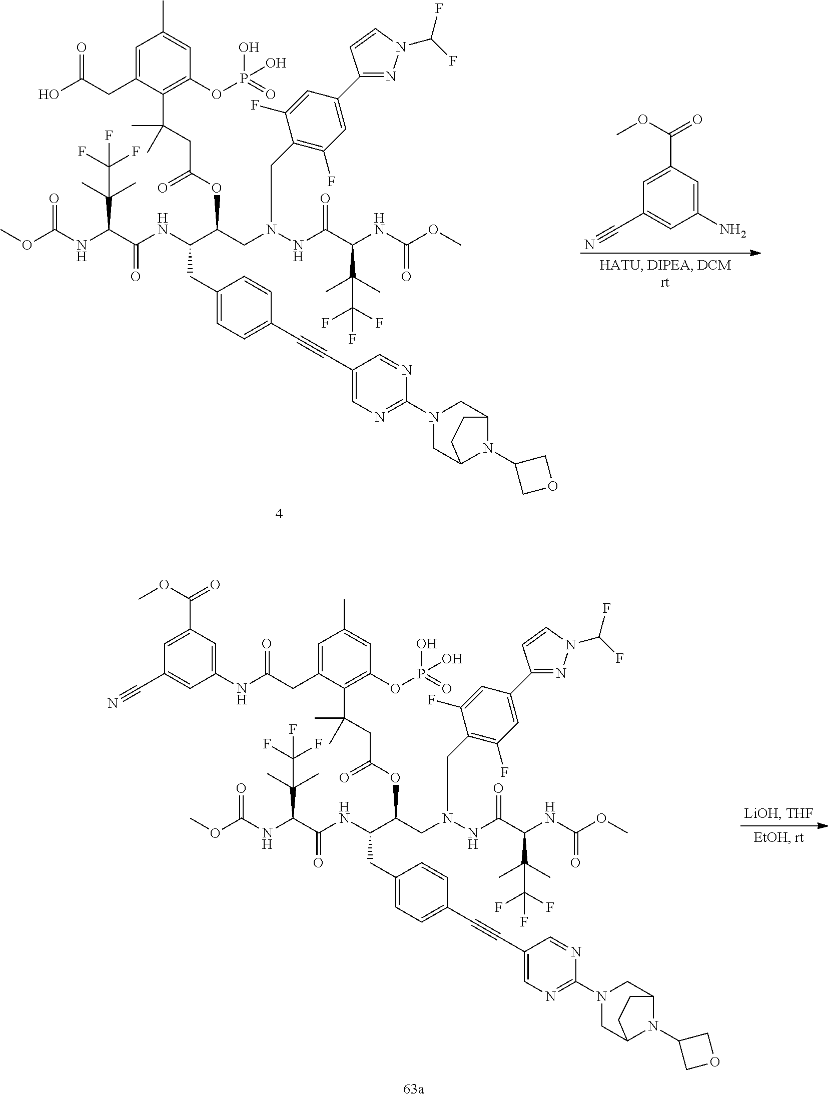 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; COC(=O)c1cc(N)cc(C#N)c1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C#N)cc(C(=O)OC)c2)cc(C)cc1OP(=O)(O)O)C(C)(C)C(F)(F)F]

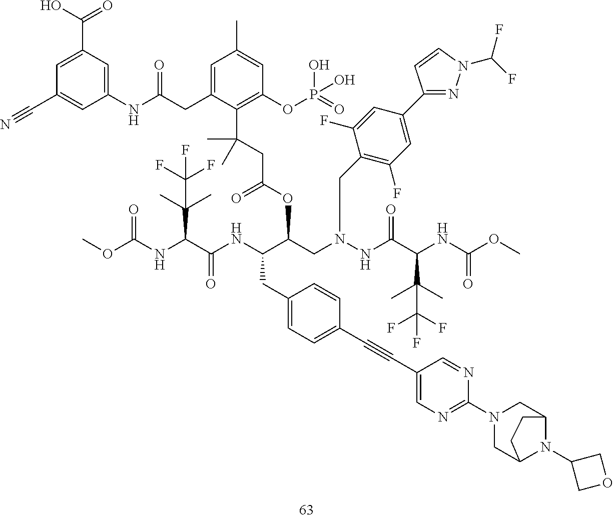 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C#N)cc(C(=O)O)c2)cc(C)cc1OP(=O)(O)O)C(C)(C)C(F)(F)F]

### Example 63

Synthesis of methyl 3-cyano-5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoate (63a): methyl 3-cyano-5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoate (63a) was synthesized using the method described for Example 48, step 1, substituting methyl 3-amino-5-cyanobenzoate (1.7 eq) for methyl 2-amino-3-(1H-indol-3-yl)propanoate (2.0 eq), HATU (1.3 eq) for HATU (1.5 eq) and reaction time increased to 24 hours.

Synthesis of 3-cyano-5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoic acid (63): 3-cyano-5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoic acid (63) was synthesized using the method described for Example 48, step 2 substituting methyl 3-cyano-5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzoate (63a) for (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)tryptophan (48a). m/z): 1629.00 [M+H]⁺. ¹H NMR (400 MHz, Methanol-d4) δ 8.53 (s, 2H), 8.38-8.28 (m), 8.14-8.08 (m), 8.00-7.95 (m), 7.71-7.30 (m), 7.26 (d, J=7.6 Hz, 2H), 7.08 (d, J=7.9 Hz, 2H), 6.96-6.87 (m), 6.84-6.72 (m), 5.08-4.94 (m), 4.78-4.73 (m), 4.38-4.24 (m), 4.23-4.12 (m), 4.11-4.02 (m), 3.95-3.85 (m), 3.74 (s, 3H), 3.66 (s, 3H), 3.58-3.45 (m), 3.10-2.92 (m), 2.91-2.78 (m), 2.41-2.30 (m), 2.30-2.18 (m), 2.12 (s, 3H), 2.06-1.98 (m), 1.95 (s, 1H), 1.82 (s, 3H), 1.68 (s, 3H), 1.27-1.11 (m), 0.99 (s, 3H). ¹⁹F NMR (376 MHz, DMSO-d₆) δ −74.85 (s), −75.33 (s), −95.07 (d, J=20.1 Hz), −112.84 (s). P NMR (162 MHz, DMSO-d₆) δ −7.96 (s).

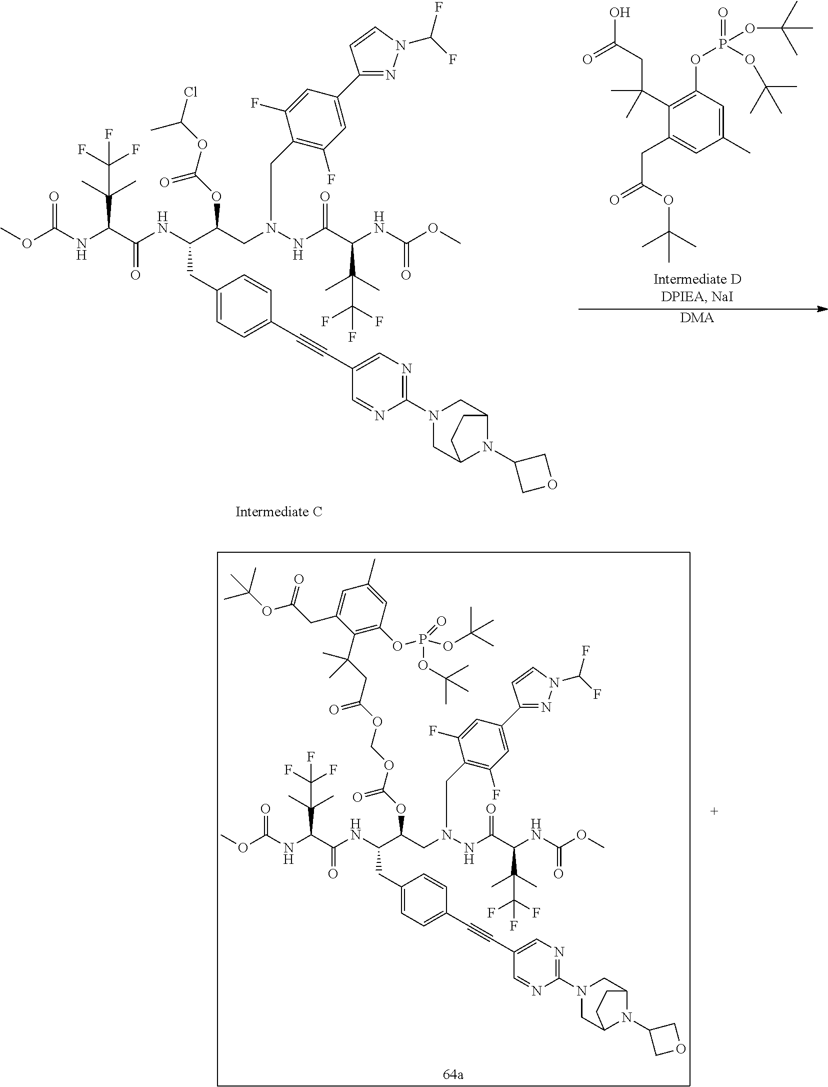

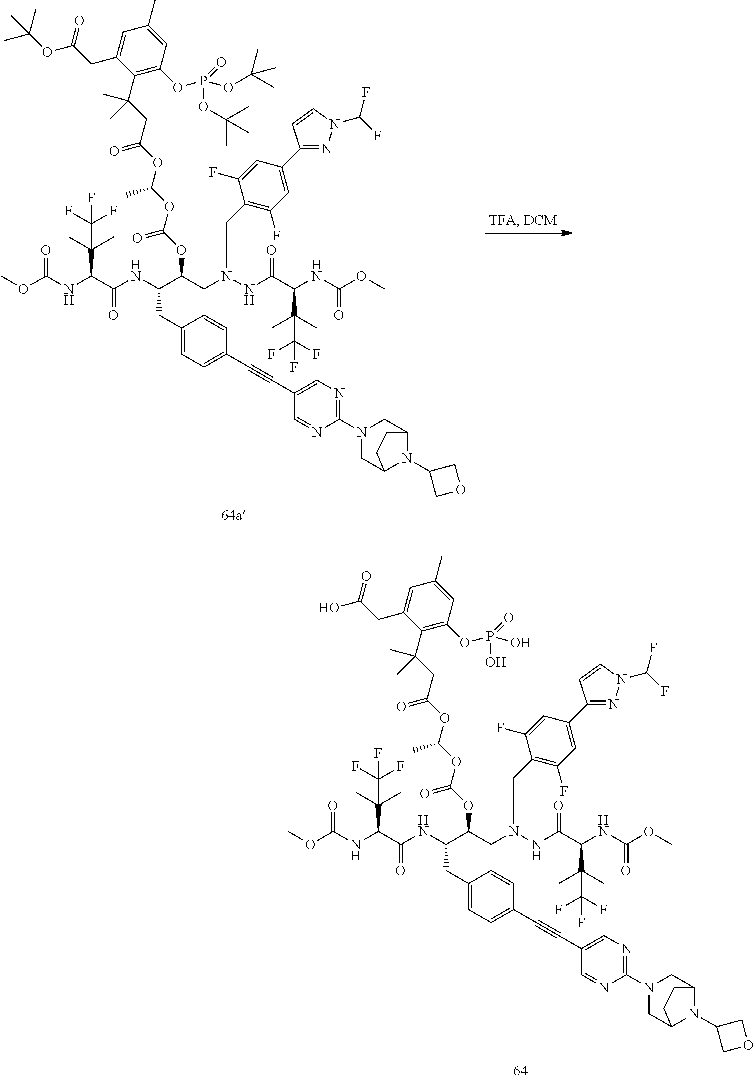 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)O[C@H](C)OC(=O)CC(C)(C)c1c(CC(=O)OC(C)(C)C)cc(C)cc1OP(=O)(OC(C)(C)C)OC(C)(C)C)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)O[C@H](C)OC(=O)CC(C)(C)c1c(CC(=O)O)cc(C)cc1OP(=O)(O)O)C(C)(C)C(F)(F)F]

### Example 64

Synthesis of (5S,10S,14S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazapentadecan-14-yl 3-(2-(2-(tert-butoxy)-2-oxoethyl)-6-((di-tert-butoxyphosphoryl)oxy)-4-methylphenyl)-3-methylbutanoate (64a) and (5S,10S,14R)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazapentadecan-14-yl 3-(2-(2-(tert-butoxy)-2-oxoethyl)-6-((di-tert-butoxyphosphoryl)oxy)-4-methylphenyl)-3-methylbutanoate (64a′): A flask charged with Intermediate C (0.40 mmol), 3-(2-(2-(tert-butoxy)-2-oxoethyl)-6-((di-tert-butoxyphosphoryl)oxy)-4-methylphenyl)-3-methylbutanoic acid Intermediate D (0.79 mmol), sodium iodide (1.6 mmol) and N,N-diisopropylethylamine (0.79 mmol) in DCE (3 mL) was heated to 70° C. for 4 hours. The reaction was cooled to room temperature and concentrated under the reduced pressure. The residue obtained was diluted with DMF, quenched with saturated aqueous sodium thiosulfate solution and purified by reverse-phase HPLC (eluent: water/MeCN 0.1% TFA) to afford (5S,10S,14S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazapentadecan-14-yl 3-(2-(2-(tert-butoxy)-2-oxoethyl)-6-((di-tert-butoxyphosphoryl)oxy)-4-methylphenyl)-3-methylbutanoate (65). m/z): 870.40 [M+2H]²⁺. and (5S,10S,14R)-8-(4-(1-(difluoromethyl)-1H-pyrazol-3-yl)-2,6-difluorobenzyl)-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazapentadecan-14-yl 3-(2-(2-(tert-butoxy)-2-oxoethyl)-6-((di-tert-butoxyphosphoryl)oxy)-4-methylphenyl)-3-methylbutanoate (64a′). 4a: MS (m/z): 870.40 [M+2H]²⁺. 64a′: MS (m/z): 870.80 [M+2H]²⁺. The Stereochemistry of 64a and 64a′ was arbitrarily assigned.

Synthesis of 2-(2-((5S,10S,14S)-8-(4-(1-(difluoromethyl)-1H-pyrazol-3-yl)-2,6-difluorobenzyl)-14,18-dimethyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetic acid (64): To a solution of (5S,10S,14S)-8-(4-(1-(difluoromethyl)-1H-pyrazol-3-yl)-2,6-difluorobenzyl)-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazapentadecan-14-yl 3-(2-(2-(tert-butoxy)-2-oxoethyl)-6-((di-tert-butoxyphosphoryl)oxy)-4-methylphenyl)-3-methylbutanoate (64a) (0.057 mmol) in DCM (3 mL) was added TFA (1.5 mL). The reaction mixture was stirred at room temperature for 30 minutes, then concentrated. The residue was purified by reverse-phase HPLC (eluent: water/MeCN 0.1% TFA) to provide 2-(2-((5S,10S,14S)-8-(4-(1-(difluoromethyl)-1H-pyrazol-3-yl)-2,6-difluorobenzyl)-14,18-dimethyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetic acid 64. MS (m/z): 786.40 [M+2H]²⁺. ¹H NMR (400 MHz, Methanol-d4) δ 8.55 (s, 2H), 8.12 (d, J=2.8 Hz, 1H), 7.72-7.39 (m), 7.39-7.33 (m), 7.28-7.19 (m), 7.01-6.88 (m), 6.72-6.67 (m), 6.67-6.60 (m), 5.01-4.95 (m), 4.84-4.74 (m), 4.54-4.52 (m), 4.35-4.26 (m), 4.20-4.12 (m), 4.07-3.96 (m), 3.95-3.85 (m), 3.72 (s, 3H), 3.68 (s, 3H), 3.52-3.48 (m), 3.28-3.26 (m), 3.00-2.88 (m), 2.86-2.68 (m), 2.25-2.21 (m), 2.03-2.01 (m), 1.65-1.62 (m), 1.39-1.37 (m), 1.22-1.19 (m), 1.00 (s, 3H). ¹⁹F NMR (376 MHz, Methanol-d4) δ −77.32 (s), −76.72 (s), −96.96 (dd, J=59.7, 3.8 Hz), −114.76 (s).

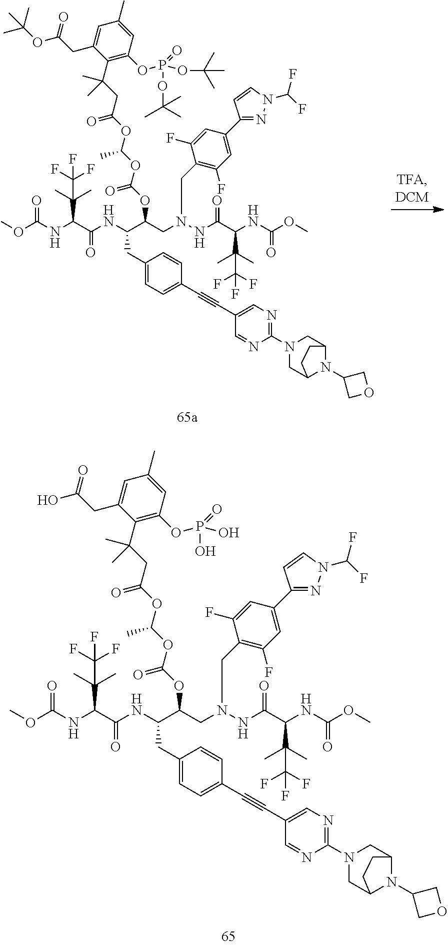 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)O[C@H](C)OC(=O)CC(C)(C)c1c(CC(=O)OC(C)(C)C)cc(C)cc1OP(=O)(OC(C)(C)C)OC(C)(C)C)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)O[C@H](C)OC(=O)CC(C)(C)c1c(CC(=O)O)cc(C)cc1OP(=O)(O)O)C(C)(C)C(F)(F)F]

### Example 65

To a solution of (5S,10S,14R)-8-(4-(1-(difluoromethyl)-1H-pyrazol-3-yl)-2,6-difluorobenzyl)-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazapentadecan-14-yl 3-(2-(2-(tert-butoxy)-2-oxoethyl)-6-((di-tert-butoxyphosphoryl)oxy)-4-methylphenyl)-3-methylbutanoate (65a) (0.046 mmol) in DCM (3 mL) was added TFA (1.5 mL). The reaction mixture was stirred at room temperature for 30 minutes, then concentrated. The residue was purified by reverse-phase HPLC (eluent: water/MeCN 0.1% TFA) to provide 2-(2-((5S,10S,14R)-8-(4-(1-(difluoromethyl)-1H-pyrazol-3-yl)-2,6-difluorobenzyl)-14,18-dimethyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetic acid 65. MS (m/z): 786.40 [M+2H]²⁺. ¹H NMR (400 MHz, Methanol-d4) δ 8.55 (s, 2H), 8.12 (d, J=2.7 Hz, 1H), 7.72-7.39 (m), 7.37-7.35 (m), 7.27-7.24 (m), 6.98-6.92 (m), 6.82-6.79 (m), 6.72-6.70 (m), 6.65-6.61 (m), 5.02-4.95 (m), 4.55-4.44 (m), 4.36-4.26 (m), 4.19-4.13 (m), 4.09-3.93 (m), 3.88-3.84 (m), 3.72-3.65 (m), 3.51-3.46 (m), 3.04-2.83 (m), 2.81-2.67 (m), 2.26-2.20 (m), 2.06-1.98 (m), 1.67-1.57 (m), 1.36-1.32 (m), 1.24-1.12 (m), 1.05 (s, 3H). ¹⁹F NMR (376 MHz, Methanol-d4) δ −77.41 (s), −77.81 (s), −96.91 (dd, J=59.8, 4.2 Hz), −114.42 (s).

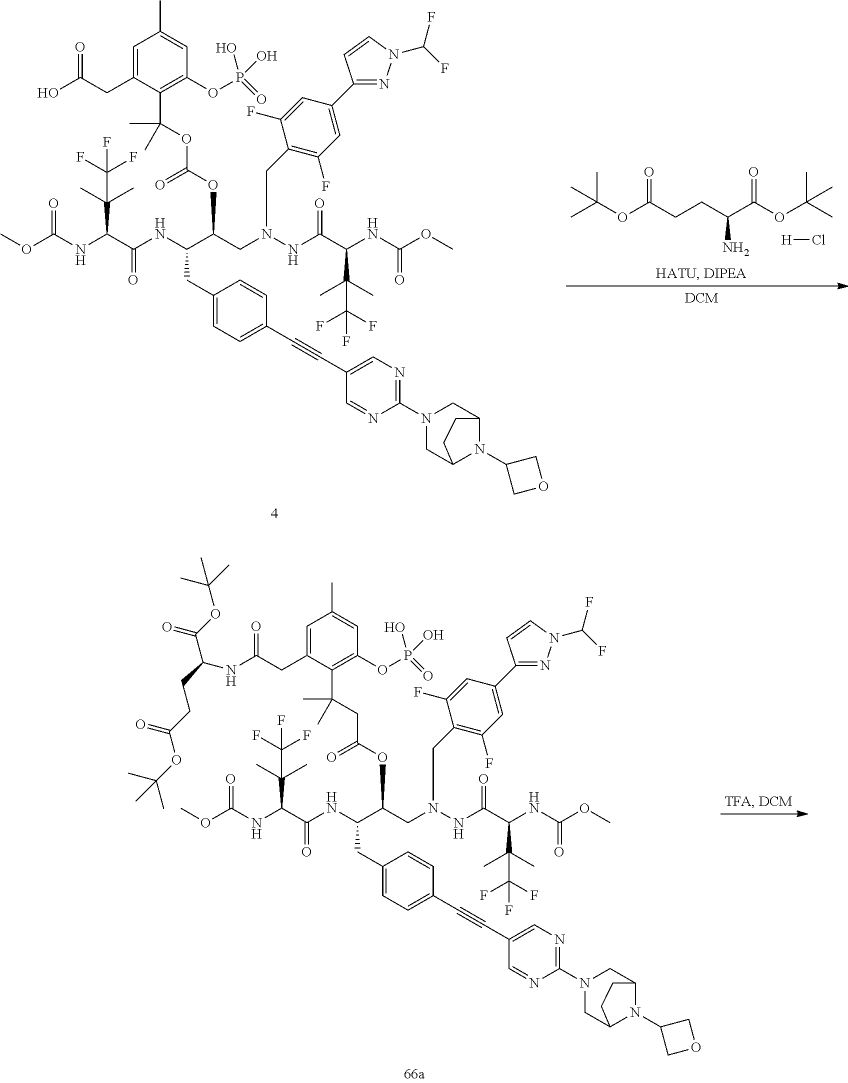

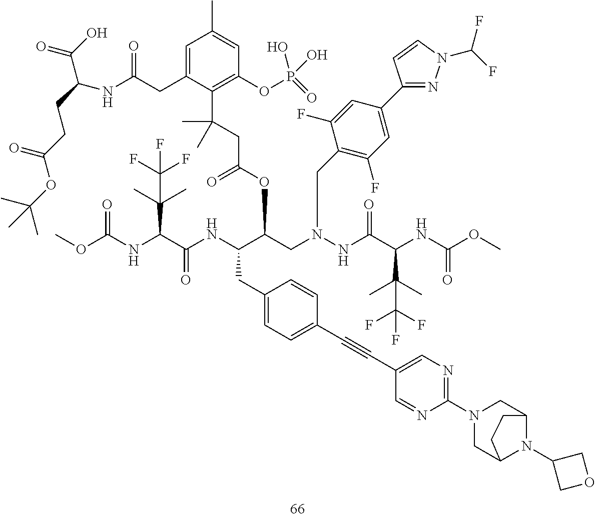 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)OC(C)(C)C)C(=O)O)cc(C)cc1OP(=O)(O)O)C(C)(C)C(F)(F)F]

### Example 66

Synthesis of di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamate (66a): To a suspension of 4 (0.044 mmol) in DCM (0.75 mL) was added a solution of di-tert-butyl L-glutamate hydrochloride (0.131 mmol), N,N-diisopropylethylamine (0.307 mmol) in DCM (0.75 mL) followed by HATU (0.175 mmol). The reaction was stirred at room temperature for 30 minutes, then concentrated. The residue obtained was diluted with acetonitrile and purified by reverse-phase HPLC (eluent: water/MeCN 0.1% TFA) to afford di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamate (66a). MS (m/z): 862.90 [M+2H]²⁺.

Synthesis of (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamic acid (66): To a solution of di-tert-butyl (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamate (66a) (0.026 mmol) in DCM (1 mL) was added TFA (1.5 mL). The reaction mixture was stirred at room temperature for 1 h, then concentrated. The residue was purified by reverse-phase HPLC (eluent: water/MeCN 0.1% TFA) to provide (2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamic acid 66. MS (m/z): 806.90 [M+2H]²⁺. ¹H NMR (400 MHz, Methanol-d4) δ 8.54 (s, 2H), 8.12 (d, J=2.7 Hz, 1H), 7.72-7.39 (m), 7.34-7.28 (m), 7.28-7.24 (m), 7.14-7.08 (m), 6.98-6.90 (m), 6.79-6.69 (m), 5.02-4.94 (m), 4.86-4.81 (m), 4.81-4.75 (m), 4.61-4.50 (m), 4.41-4.26 (m), 4.26-4.14 (m), 3.99-3.91 (m), 3.82-3.67 (m), 3.55-3.39 (m), 3.00-2.93 (m), 2.89-2.74 (m), 2.46-2.38 (m), 2.38-2.28 (m), 2.28-2.17 (m), 2.14-2.07 (m), 2.06-1.93 (m), 1.79 (s, 3H), 1.65 (s, 3H), 1.24-1.15 (m), 1.01 (s, 3H). ¹⁹F NMR (376 MHz, Methanol-d4) δ −77.20 (s), −77.79 (s), −96.87 (d, J=5.2 Hz), −97.03 (d, J=5.2 Hz), −114.42 (s).

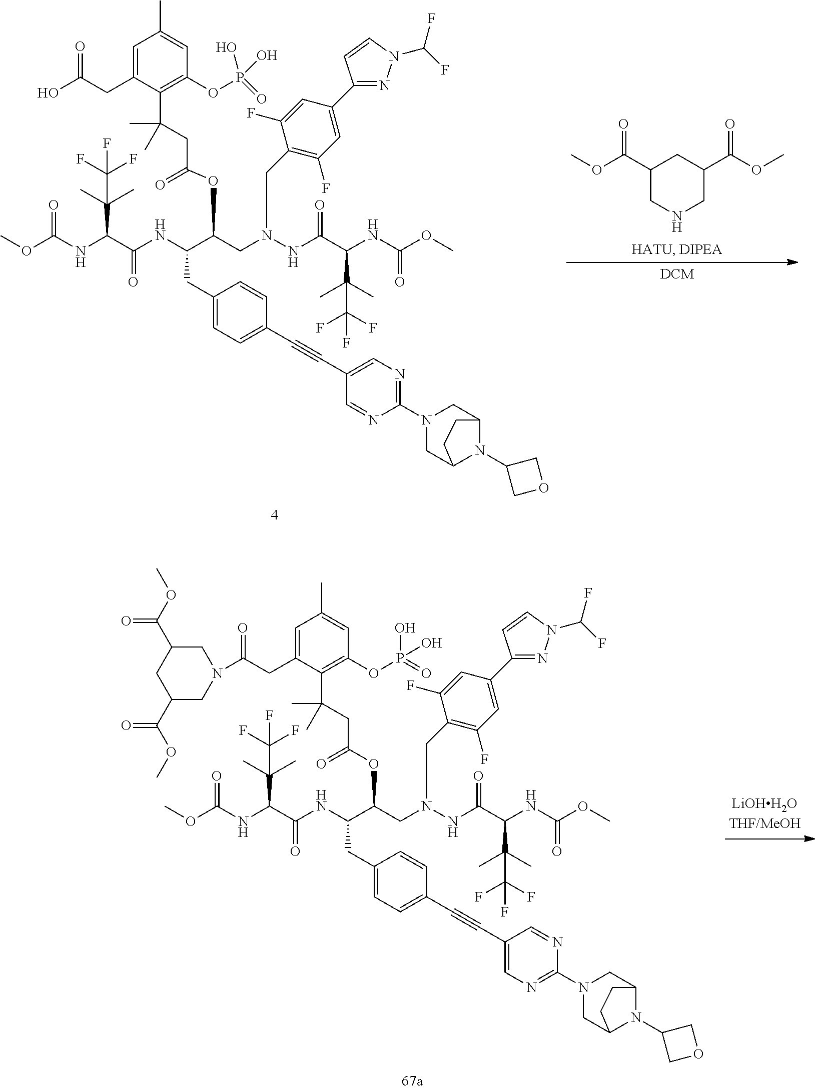

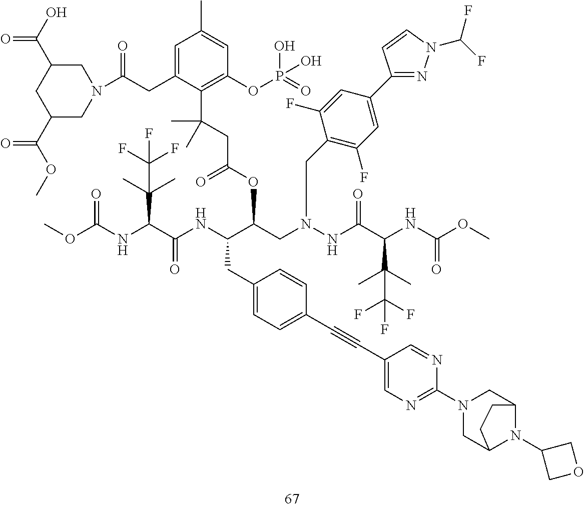 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC(C(=O)O)CC(C(=O)OC)C2)cc(C)cc1OP(=O)(O)O)C(C)(C)C(F)(F)F]

### Example 67

Synthesis of dimethyl 1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperidine-3,5-dicarboxylate (67a): To a suspension of 4 (0.063 mmol) in DCM (1 mL) was added a solution of dimethyl piperidine-3,5-dicarboxylate (0.131 mmol), N,N-diisopropylethylamine (0.250 mmol) in DCM (1 mL) followed by HATU (0.066 mmol). The reaction was stirred at room temperature for 30 minutes, then concentrated. The residue obtained was diluted with acetonitrile and purified by reverse-phase HPLC (eluent: water/MeCN 0.1% TFA) to afford dimethyl 1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperidine-3,5-dicarboxylate (67a). MS (m/z): 834.20 [M+2H]²⁺.

Synthesis of 1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperidine-3,5-dicarboxylic acid (67): To a solution of dimethyl 1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperidine-3,5-dicarboxylate 67a (0.043 mmol) in a mixture of 1:1 MeOH/THF (2 mL) was added 0.5M aqueous lithium hydroxide solution (0.864 mL) and stirred at room temperature for 4 h. The reaction mixture was neutralized with dilute acetic acid and then purified by reverse-phase HPLC (eluent: water/MeCN 0.1% TFA) to afford 1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperidine-3,5-dicarboxylic acid 67. MS (m/z): 820.20 [M+2H]²⁺. ¹H NMR (400 MHz, Methanol-d4) δ 8.54 (s, 2H), 8.12 (d, J=2.7 Hz, 1H), 7.72-7.39 (m), 7.34-7.21 (m), 7.17-7.07 (m), 6.99-6.85 (m), 6.74-6.55 (m), 5.02-4.93 (m), 4.86-4.74 (m), 4.57-4.36 (m), 4.34-4.26 (m), 4.26-4.13 (m), 3.99-3.82 (m), 3.82-3.66 (m), 3.54-3.39 (m), 3.27-3.09 (m), 3.05-2.85 (m), 2.84-2.74 (m), 2.71-2.60 (m), 2.58-2.44 (m), 2.33-2.20 (m), 2.12-1.97 (m), 1.81-1.68 (m), 1.66-1.59 (m), 1.24-1.16 (m), 1.06-0.97 (m). ¹⁹F NMR (376 MHz, Methanol-d4) δ −77.13-−77.29 (m), −77.66-−77.86 (m), −96.82-−96.90 (m), −97.01-−97.03 (m), −114.10-−114.61 (m).

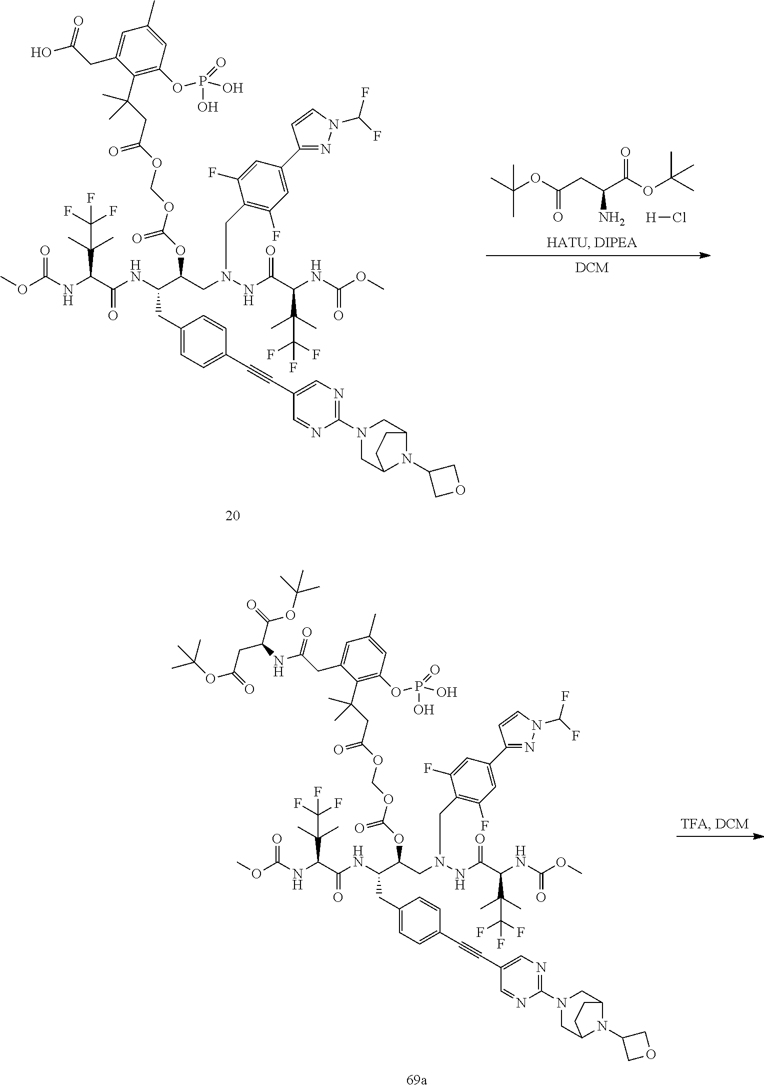

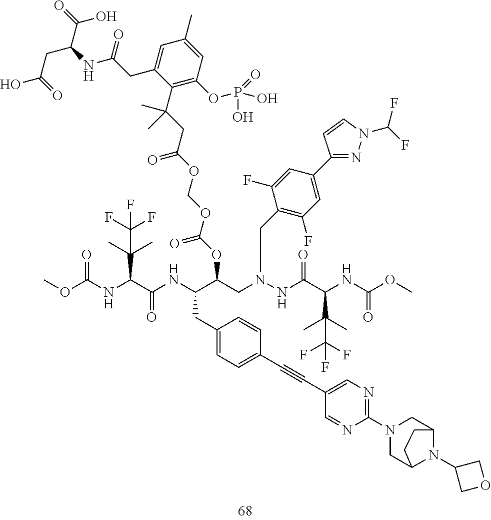 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CC(=O)O)C(=O)O)cc(C)cc1OP(=O)(O)O)C(C)(C)C(F)(F)F]

### Example 68

(2-(2-((3S,8S)-6-(4-(1-(difluoromethyl)-1H-pyrazol-3-yl)-2,6-difluorobenzyl)-1,1,1-trifluoro-3-((methoxycarbonyl)amino)-2,2,16-trimethyl-8-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-4,10,14-trioxo-9,11,13-trioxa-5,6-diazaheptadecan-16-yl)-5-methyl-3-(phosphonooxy)phenyl)acetyl)-L-aspartic acid 68 was synthesized using the method described in Example 66, substituting di-tert-butyl L-aspartate hydrochloride for di-tert-butyl L-glutamate hydrochloride and substituting 20 for 4. MS (m/z): 836.90 [M+2H]²⁺. ¹H NMR (400 MHz, Methanol-d4) δ 8.55 (s, 2H), 8.12 (d, J=2.8 Hz, 1H), 7.71-7.39 (m), 7.38-7.33 (m), 7.32-7.22 (m), 7.00 (d, J=10.1 Hz, 1H), 6.95 (d, J=2.8 Hz, 1H), 6.80-6.72 (m), 5.75 (d, J=5.9 Hz, 1H), 5.64 (d, J=5.9 Hz, 1H), 5.02-4.95 (m), 4.67-4.58 (m), 4.57-4.43 (m), 4.38-4.26 (m), 4.18-4.04 (m), 3.96-3.85 (m), 3.74-3.65 (m), 3.55-3.44 (m), 3.04-2.82 (m), 2.81-2.70 (m), 2.28-2.17 (m), 2.06-1.99 (m), 1.64-1.62 (m), 1.25-1.12 (m), 1.08 (s, 3H). ¹⁹F NMR (376 MHz, Methanol-d4) δ −77.37 (s), −77.67 (s), −96.85 (d, J=4.1 Hz), −97.01 (d, J=4.2 Hz), −114.61 (s).

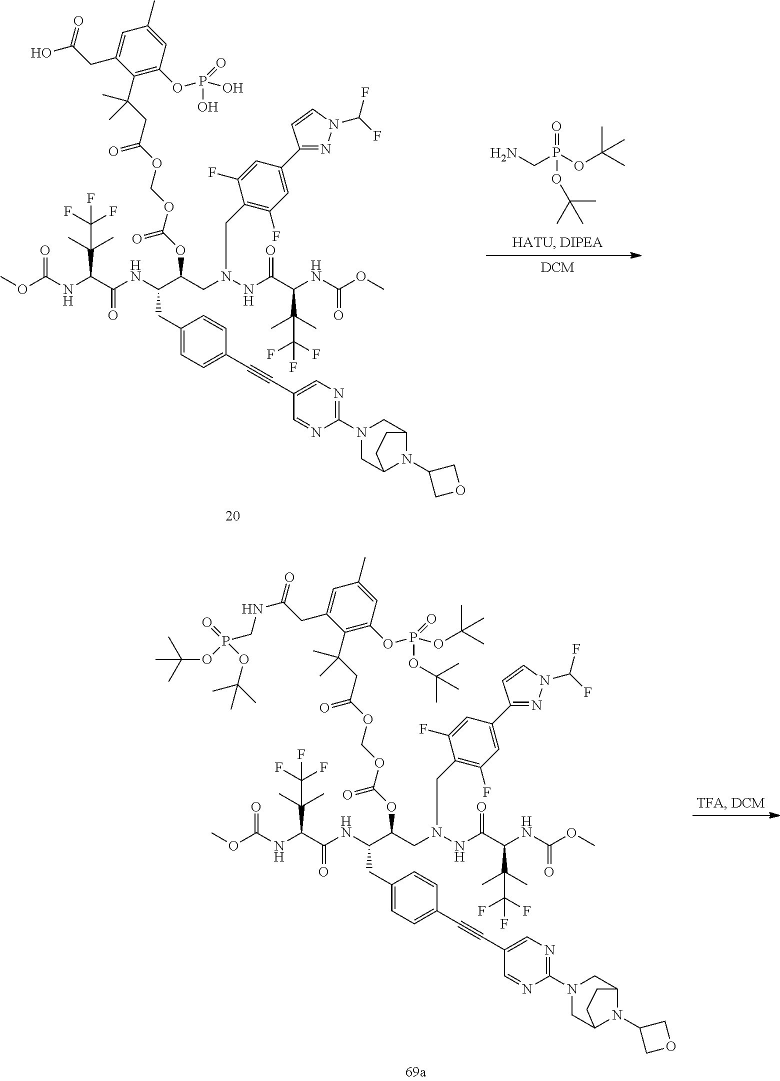

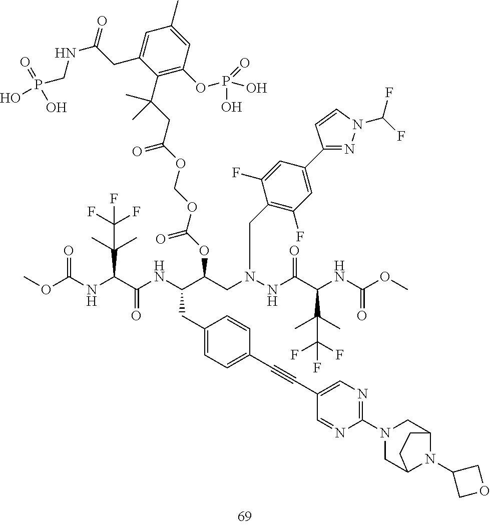 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCP(=O)(O)O)cc(C)cc1OP(=O)(O)O)C(C)(C)C(F)(F)F]

### Example 69

((2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetamido)methyl)phosphonic acid 69 was synthesized using the method described in Example 66, substituting di-tert-butyl (aminomethyl)phosphonate for di-tert-butyl L-glutamate hydrochloride and substituting 20 for 4. MS (m/z): 825.90 [M+2H]²⁺. ¹H NMR (400 MHz, Methanol-d4) δ 8.55 (s, 2H), 8.12 (d, J=2.7 Hz, 1H), 7.71-7.39 (m), 7.38-7.36 (m), 7.30-7.20 (m), 7.04-6.91 (m), 6.89-6.73 (m), 5.75 (d, J=5.9 Hz, 1H), 5.63 (d, J=5.9 Hz, 1H), 5.02-4.94 (m), 4.65-4.48 (m), 4.38-4.25 (m), 4.20-4.05 (m), 3.95-3.93 (m), 3.73-3.66 (m), 3.66-3.58 (m), 3.56-3.45 (m), 3.21-3.10 (m), 3.03-2.86 (m), 2.80-2.70 (m), 2.28-2.15 (m), 2.05-1.95 (m), 1.66-1.58 (m), 1.24-1.09 (m), 1.07 (s, 3H). ¹⁹F NMR (376 MHz, Methanol-d4) δ −77.37 (s), −77.67 (s), −96.85 (d, J=5.4 Hz), −97.01 (d, J=5.5 Hz), −114.64 (s).

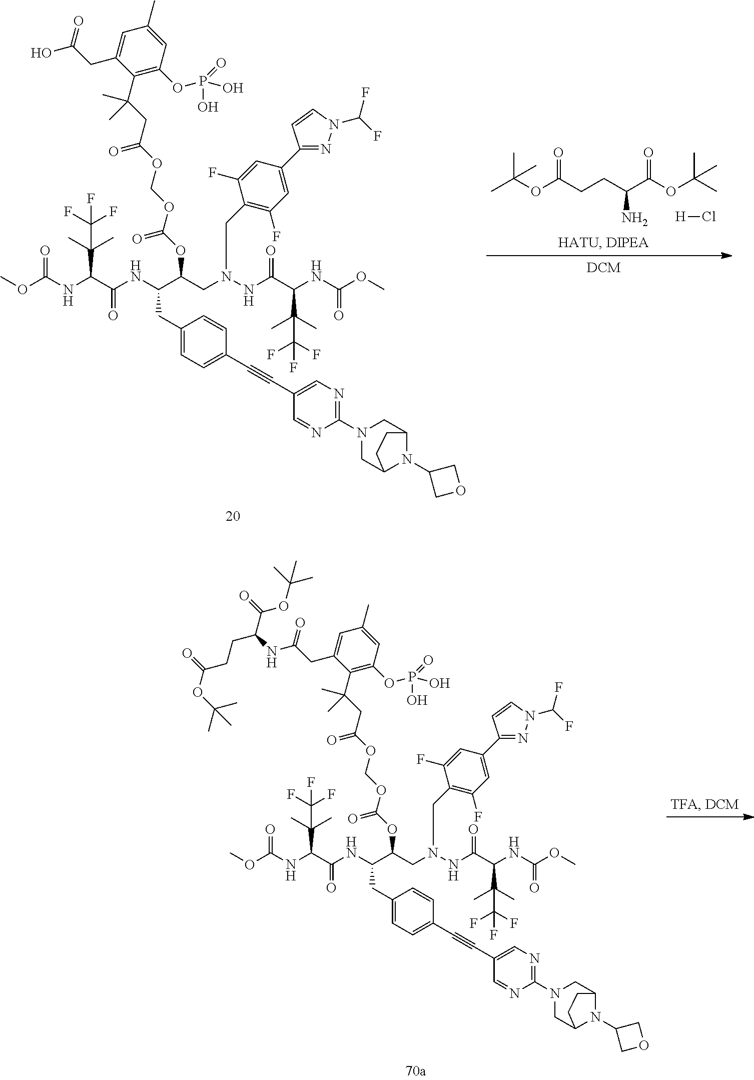

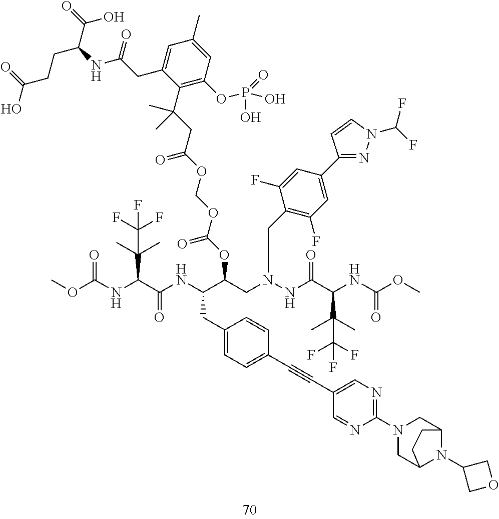 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CCC(=O)O)C(=O)O)cc(C)cc1OP(=O)(O)O)C(C)(C)C(F)(F)F]

### Example 70

(2-(2-((3S,8S)-6-(4-(1-(difluoromethyl)-1H-pyrazol-3-yl)-2,6-difluorobenzyl)-1,1,1-trifluoro-3-((methoxycarbonyl)amino)-2,2,16-trimethyl-8-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-4,10,14-trioxo-9,11,13-trioxa-5,6-diazaheptadecan-16-yl)-5-methyl-3-(phosphonooxy)phenyl)acetyl)-L-glutamic acid 70 was synthesized using the method described in Example 66, substituting 20 for 4. MS (m/z): 843.90 [M+2H]²⁺. ¹H NMR (400 MHz, Methanol-d4) δ 8.55 (s, 2H), 8.12 (d, J=2.7 Hz, 1H), 7.72-7.39 (m), 7.39-7.31 (m), 7.30-7.19 (m), 7.03-6.91 (m), 6.84-6.74 (m), 5.74 (d, J=5.9 Hz, 1H), 5.64 (d, J=5.9 Hz, 1H), 5.02-4.94 (m), 4.65-4.45 (m), 4.37-4.26 (m), 4.18-4.13 (m), 4.13-3.87 (m), 3.74-3.66 (m), 3.54-3.45 (m), 3.26-3.10 (m), 3.03-2.87 (m), 2.80-2.70 (m), 2.43-2.35 (m), 2.28-2.15 (m), 2.07-1.91 (m), 1.68-1.60 (m), 1.25-1.12 (m), 1.08 (s, 3H). ¹⁹F NMR (376 MHz, Methanol-d4) δ −77.38 (s), −77.66 (s), −96.85 (d, J=5.5 Hz), −97.01 (d, J=5.6 Hz), −114.61 (s).

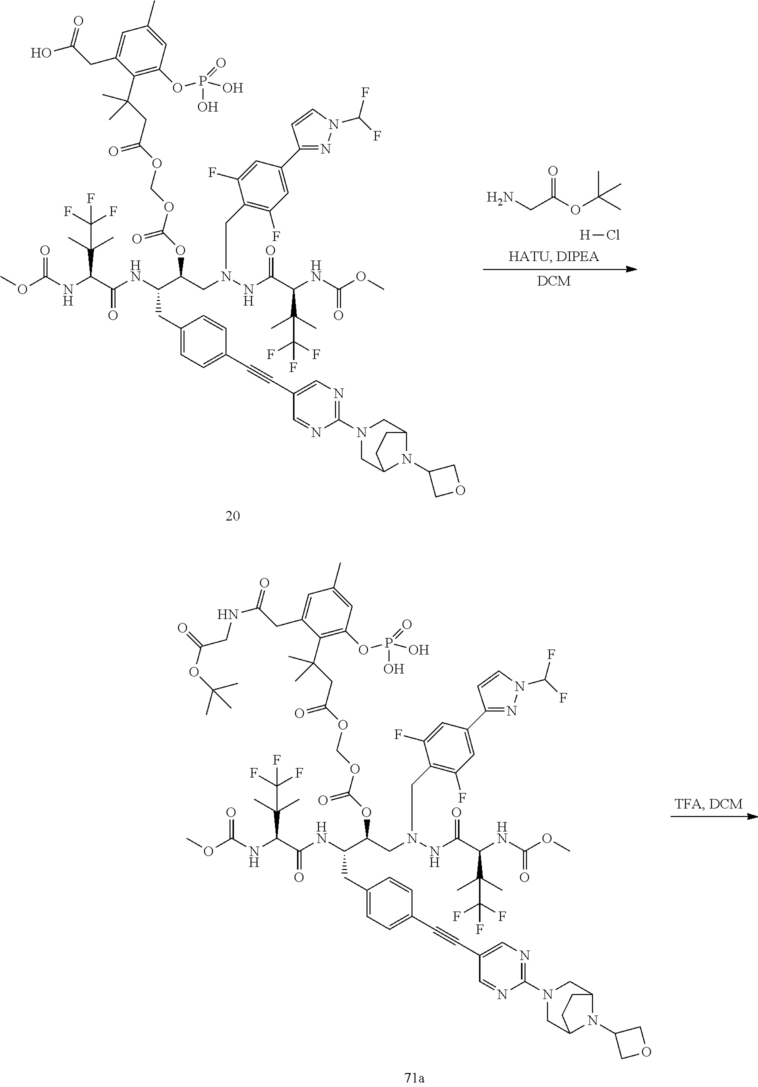

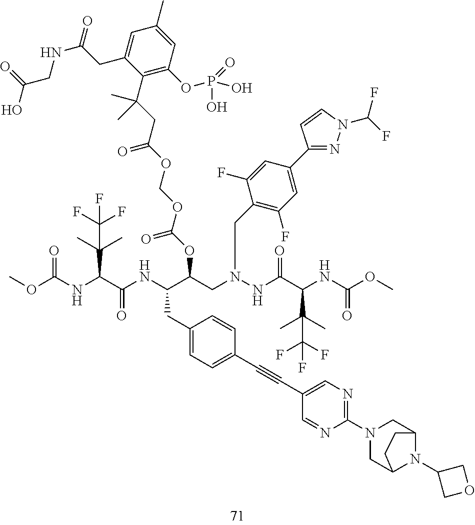 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCC(=O)O)cc(C)cc1OP(=O)(O)O)C(C)(C)C(F)(F)F]

### Example 71

Compound (2-(2-((3S,8S)-6-(4-(1-(difluoromethyl)-1H-pyrazol-3-yl)-2,6-difluorobenzyl)-1,1,1-trifluoro-3-((methoxycarbonyl)amino)-2,2,16-trimethyl-8-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-4,10,14-trioxo-9,11,13-trioxa-5,6-diazaheptadecan-16-yl)-5-methyl-3-(phosphonooxy)phenyl)acetyl)glycine 71 was synthesized using the method described in Example 66, substituting tert-butyl glycinate hydrochloride for di-tert-butyl L-glutamate hydrochloride and substituting 20 for 4. MS (m/z): 807.90 [M+2H]²⁺. ¹H NMR (400 MHz, Methanol-d4) δ 8.55 (s, 2H), 8.12 (d, J=2.8 Hz, 1H), 7.72-7.39 (m), 7.39-7.32 (m), 7.32-7.19 (m), 7.04-6.90 (m), 6.84-6.75 (m), 5.75 (d, J=6.0 Hz, 1H), 5.63 (d, J=5.9 Hz, 1H), 5.02-4.93 (m), 4.65-4.46 (m), 4.37-4.26 (m), 4.20-4.04 (m), 3.97-3.90 (m), 3.74-3.60 (m), 3.54-3.45 (m), 3.25-3.08 (m), 3.02-2.85 (m), 2.80-2.70 (m), 2.28-2.19 (m), 2.06-1.93 (m), 1.67-1.60 (m), 1.25-1.10 (m), 1.07 (s, 3H). ¹⁹F NMR (376 MHz, Methanol-d4) δ −77.38 (s), −77.66 (s), −96.85 (d, J=5.5 Hz), −97.01 (d, J=5.6 Hz), −114.73 (s).

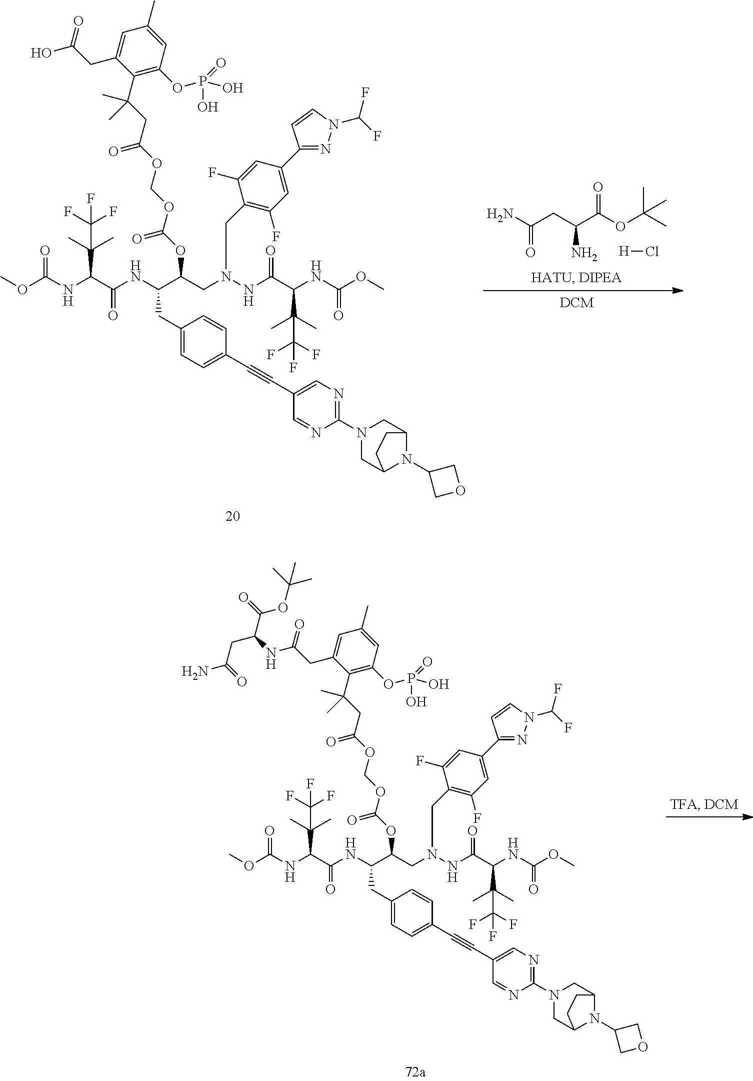

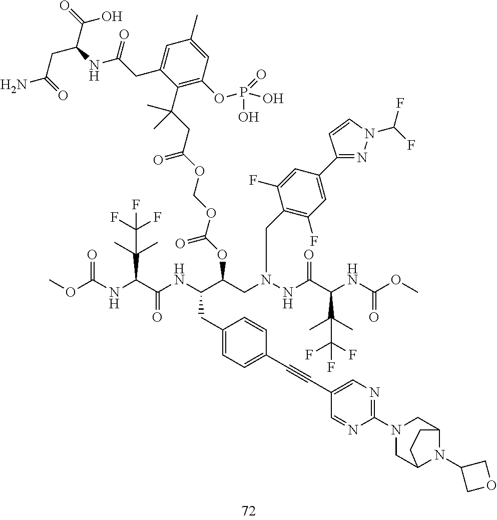 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N[C@@H](CC(N)=O)C(=O)O)cc(C)cc1OP(=O)(O)O)C(C)(C)C(F)(F)F]

### Example 72

(2-(2-((3S,8S)-6-(4-(1-(difluoromethyl)-1H-pyrazol-3-yl)-2,6-difluorobenzyl)-1,1,1-trifluoro-3-((methoxycarbonyl)amino)-2,2,16-trimethyl-8-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-4,10,14-trioxo-9,11,13-trioxa-5,6-diazaheptadecan-16-yl)-5-methyl-3-(phosphonooxy)phenyl)acetyl)-L-asparagine 72 was synthesized using the method described in Example 66, substituting tert-butyl L-asparaginate hydrochloride for di-tert-butyl L-glutamate hydrochloride and substituting 20 for 4. MS (m/z): 836.40 [M+2H]²⁺. ¹H NMR (400 MHz, Methanol-d4) δ 8.55 (s, 1H), 8.12 (d, J=2.8 Hz, 1H), 7.72-7.39 (m), 7.39-7.32 (m), 7.31-7.21 (m), 7.05-6.92 (m), 6.83-6.74 (m), 5.74 (d, J=6.0 Hz, 1H), 5.65 (d, J=6.0 Hz, 1H), 5.02-4.94 (m), 4.66-4.47 (m), 4.39-4.26 (m), 4.20-4.05 (m), 4.00-3.84 (m), 3.74-3.65 (m), 3.53-3.45 (m), 3.10-3.02 (m), 3.02-2.87 (m), 2.86-2.70 (m), 2.28-2.16 (m), 2.06-1.93 (m), 1.66-1.55 (m), 1.26-1.11 (m), 1.08 (s, 3H). ¹⁹F NMR (376 MHz, Methanol-d4) δ −77.37 (s), −77.83 (s), −96.85 (d, J=5.5 Hz), −97.01 (d, J=5.6 Hz), −114.70 (s).

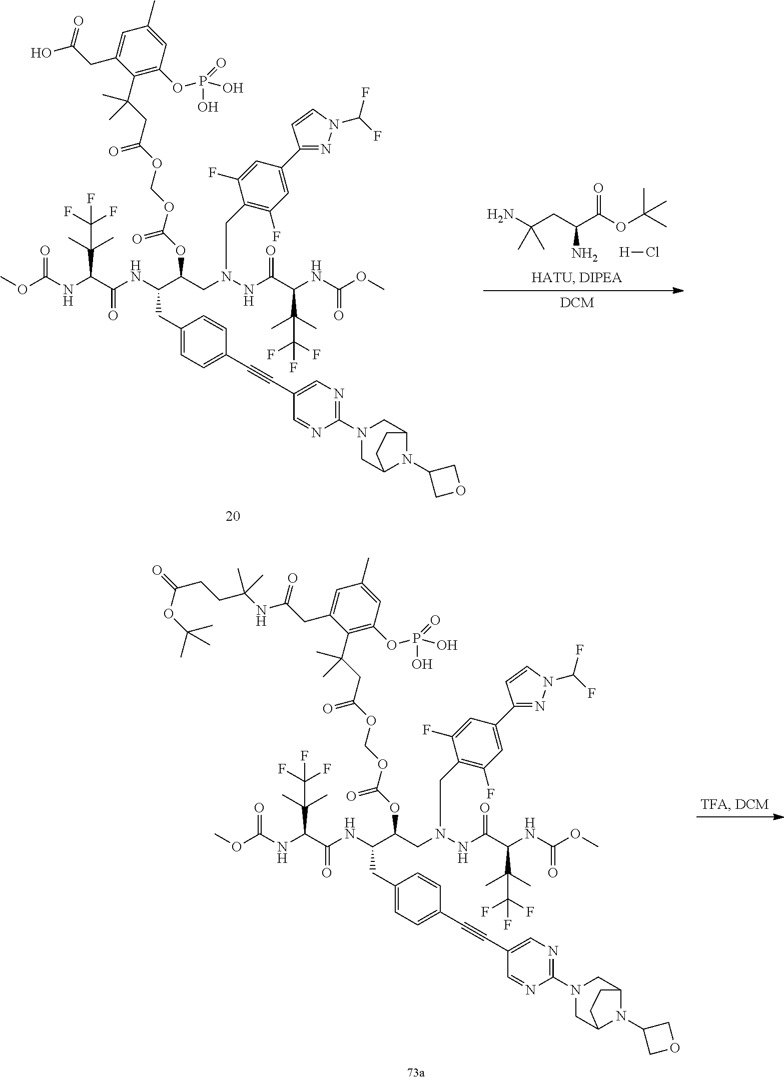

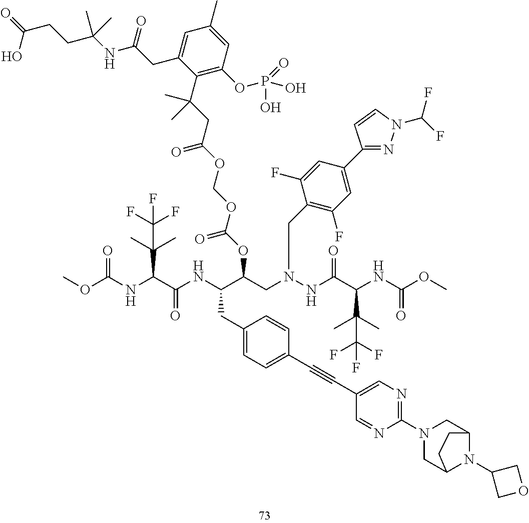 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NC(C)(C)CCC(=O)O)cc(C)cc1OP(=O)(O)O)C(C)(C)C(F)(F)F]

### Example 73

4-(2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[l3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetamido)-4-methylpentanoic acid 73 was synthesized using the method described in Example 66, substituting tert-butyl 4-amino-4-methylpentanoate hydrochloride for di-tert-butyl L-glutamate hydrochloride and substituting 20 for 4. MS (m/z): 835.90 [M+2H]²⁺. ¹H NMR (400 MHz, Methanol-d4) δ 8.55 (s, 2H), 8.12 (d, J=2.8 Hz, 1H), 7.73-7.39 (m), 7.39-7.31 (m), 7.28-7.18 (m), 7.04-6.92 (m), 6.85-6.73 (m), 5.75 (d, J=6.0 Hz, 1H), 5.64 (d, J=5.9 Hz, 1H), 5.03-4.94 (m), 4.65-4.55 (m), 4.54-4.45 (m), 4.36-4.26 (m), 4.20-4.13 (m), 4.13-4.04 (m), 3.88-3.81 (m), 3.74-3.64 (m), 3.54-3.44 (m), 3.22-3.14 (m), 3.04-2.87 (m), 2.81-2.70 (m), 2.31-2.20 (m), 2.08-1.95 (m), 1.66-1.61 (m), 1.33-1.28 (m), 1.25-1.12 (m), 1.07 (s, 3H). ¹⁹F NMR (376 MHz, Methanol-d4) δ −77.41 (s), −77.66 (s), −96.85 (d, J=5.5 Hz), −97.01 (d, J=5.6 Hz), −114.69 (s).

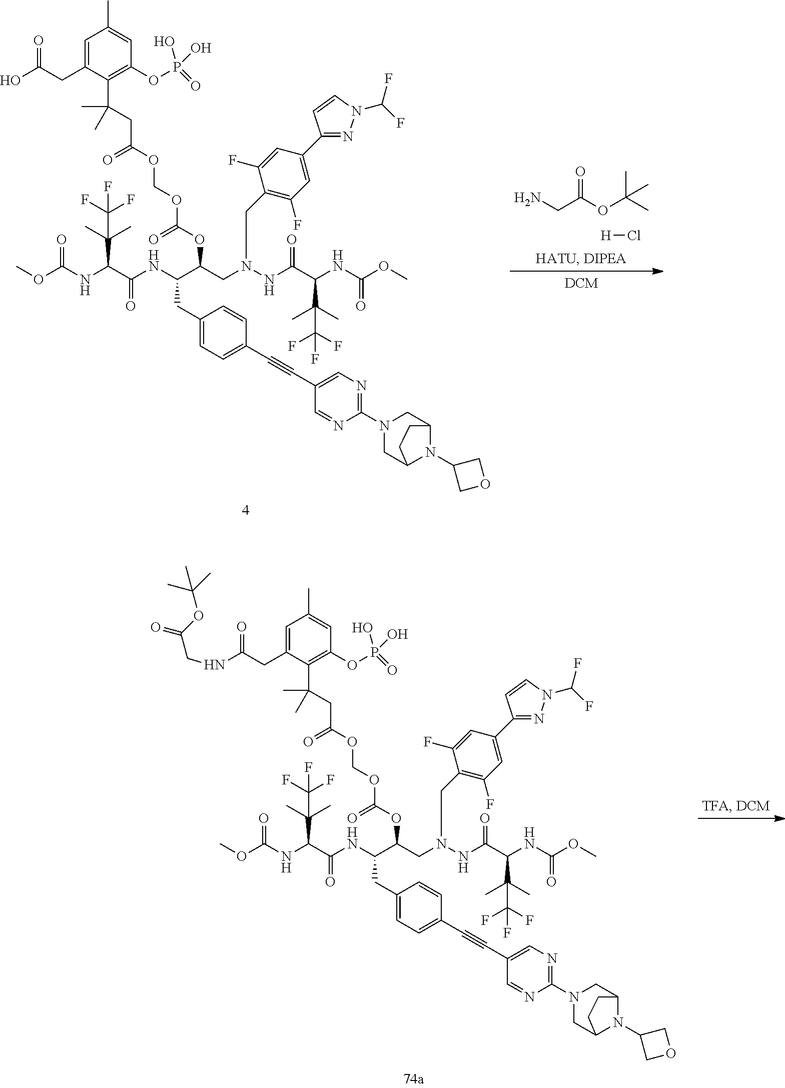

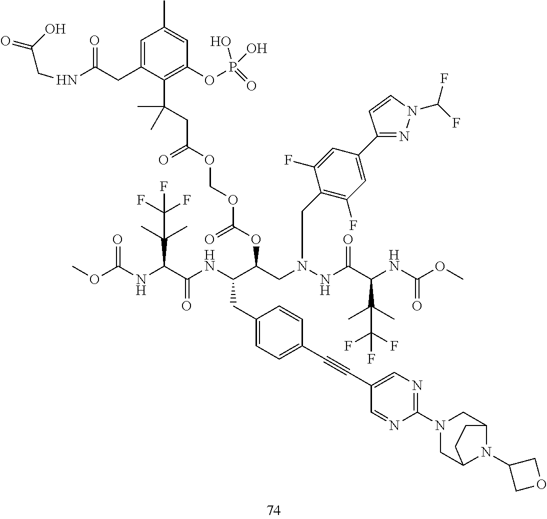 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)NCC(=O)O)cc(C)cc1OP(=O)(O)O)C(C)(C)C(F)(F)F]

### Example 74

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)glycine 74 was synthesized using the method described in Example 66, substituting tert-butyl glycinate hydrochloride for di-tert-butyl L-glutamate hydrochloride. MS (m/z): 770.90 [M+2H]²⁺. ¹H NMR (400 MHz, Methanol-d4) δ 8.54 (s, 2H), 8.12 (d, J=2.8 Hz, 1H), 7.74-7.38 (m), 7.34-7.21 (m), 7.15-7.07 (m), 6.99-6.86 (m), 6.79-6.70 (m), 5.02-4.93 (m), 4.60-4.45 (m), 4.42-4.27 (m), 4.25-4.11 (m), 4.04-3.88 (m), 3.82-3.66 (m), 3.57-3.39 (m), 3.04-2.71 (m), 2.39-2.29 (m), 2.29-2.20 (m), 2.13-2.06 (m), 2.06-1.95 (m), 1.79 (s, 3H), 1.65 (s, 3H), 1.25-1.12 (m), 1.01 (s, 3H). ¹⁹F NMR (376 MHz, Methanol-d4) δ −77.19 (s), −77.90 (s), −96.88 (d, J=5.5 Hz), −97.03 (d, J=5.6 Hz), −114.49 (s).

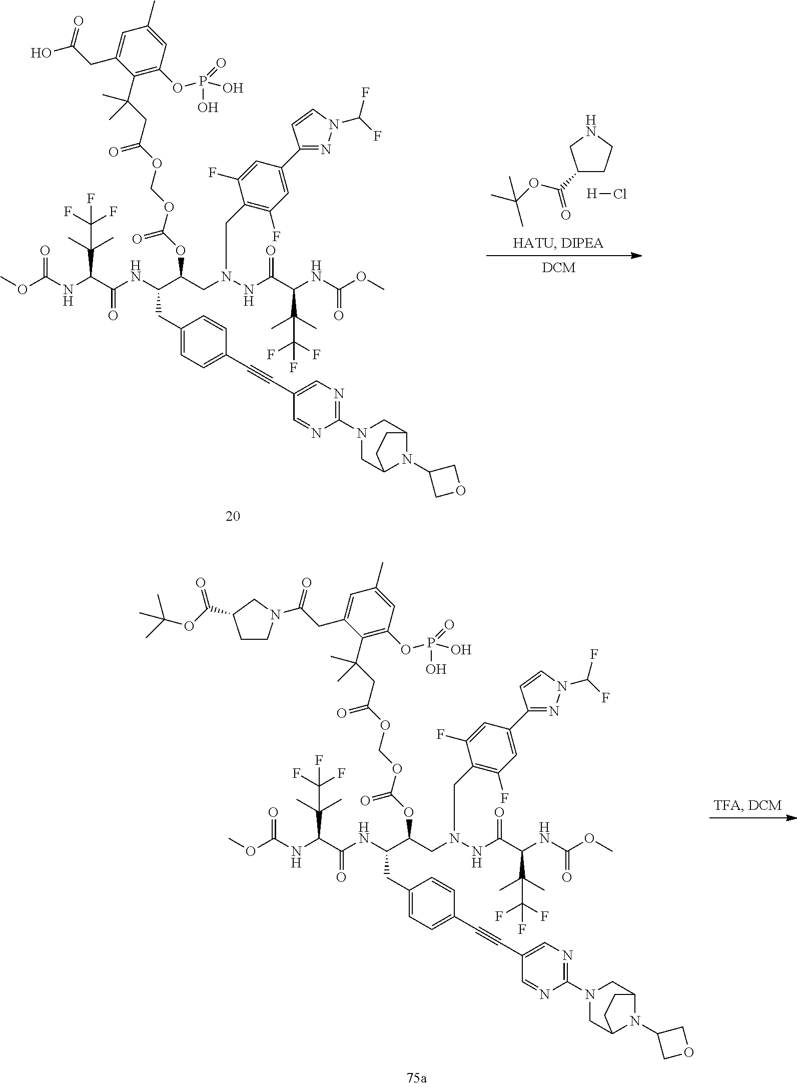

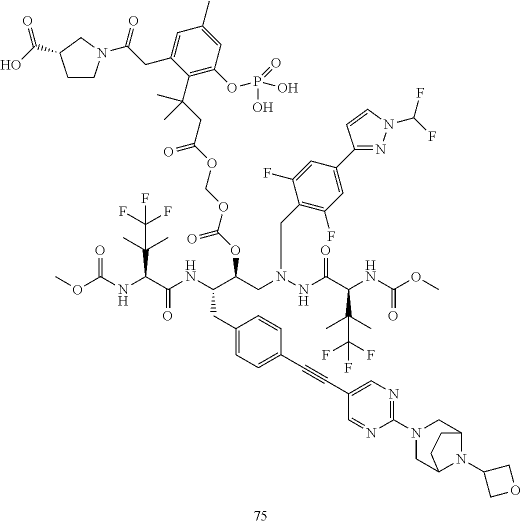 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CC[C@H](C(=O)O)C2)cc(C)cc1OP(=O)(O)O)C(C)(C)C(F)(F)F]

### Example 75

(3S)-1-(2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetyl)pyrrolidine-3-carboxylic acid 75 was synthesized using the method described in Example 66, substituting tert-butyl (S)-pyrrolidine-3-carboxylate hydrochloride for di-tert-butyl L-glutamate hydrochloride and substituting 20 for 4. MS (m/z): 828.00 [M+2H]²⁺. ¹H NMR (400 MHz, Methanol-d4) δ 8.55 (s, 2H), 8.12 (d, J=2.7 Hz, 1H), 7.73-7.39 (m), 7.39-7.32 (m), 7.28-7.15 (m), 7.04-6.92 (m), 6.85-6.78 (m), 6.67-6.61 (m), 5.80-5.72 (m), 5.67-5.59 (m), 5.02-4.94 (m), 4.65-4.44 (m), 4.36-4.26 (m), 4.19-4.12 (m), 4.12-3.94 (m), 3.76-3.63 (m), 3.63-3.45 (m), 3.28-3.20 (m), 3.20-3.08 (m), 3.04-2.86 (m), 2.80-2.69 (m), 2.28-2.11 (m), 2.07-1.95 (m), 1.67-1.57 (m), 1.24-1.12 (m), 1.07 (s, 3H). ¹⁹F NMR (376 MHz, Methanol-d4) δ −77.40 (s), −77.65 (s), −96.85 (d, J=5.5 Hz), −97.01 (d, J=5.6 Hz), −114.78 (s)

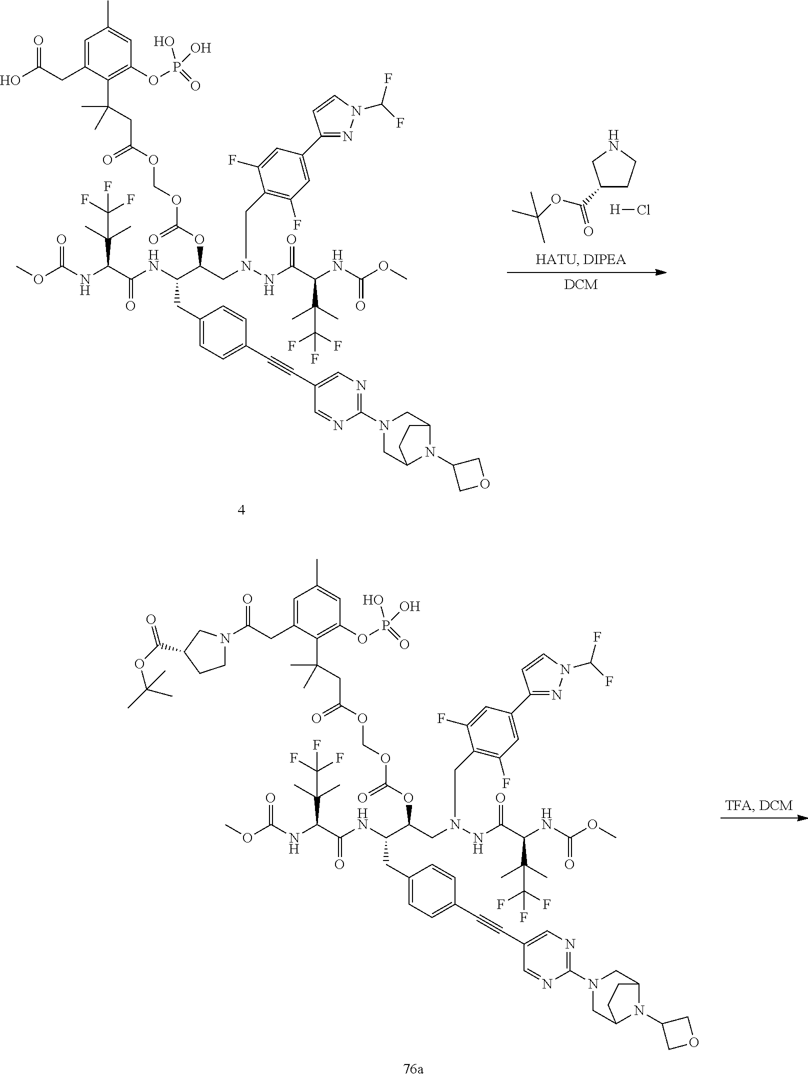

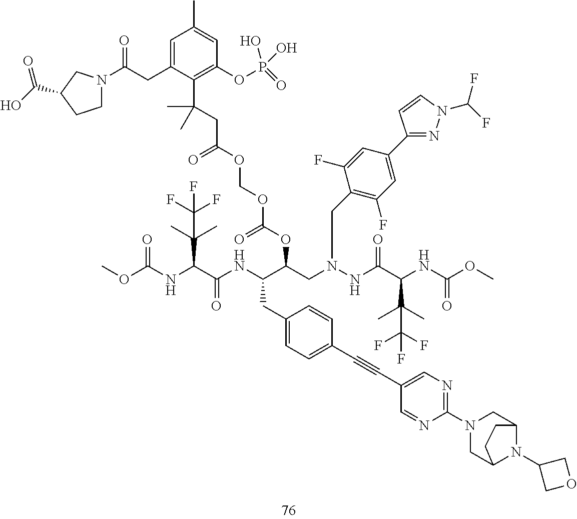 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CC[C@H](C(=O)O)C2)cc(C)cc1OP(=O)(O)O)C(C)(C)C(F)(F)F]

### Example 76

(3S)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)pyrrolidine-3-carboxylic acid 76 was synthesized using the method described in Example 66, substituting tert-butyl (S)-pyrrolidine-3-carboxylate hydrochloride for di-tert-butyl L-glutamate hydrochloride. MS (m/z): 791.00 [M+2H]²⁺. ¹H NMR (400 MHz, Methanol-d4) δ 8.54 (s, 2H), 8.12 (d, J=2.8 Hz, 1H), 7.74-7.38 (m), 7.34-7.22 (m), 7.15-7.06 (m), 7.00-6.88 (m), 6.66-6.60 (m), 5.02-4.94 (m), 4.81-4.75 (m), 4.60-4.50 (m), 4.40-4.13 (m), 3.98-3.90 (m), 3.84-3.65 (m), 3.65-3.55 (m), 3.55-3.46 (m), 3.28-3.19 (m), 3.19-3.10 (m), 3.05-2.86 (m), 2.81-2.70 (m), 2.36-2.14 (m), 2.10-1.97 (m), 1.77-1.68 (m), 1.66-1.60 (m), 1.27-1.13 (m), 1.06-0.96 (m). ¹⁹F NMR (377 MHz, Methanol-d4) δ −77.15-−77.17 (m), −77.69-−77.84 (m), −96.85-−96.88 (m), −97.01-−97.04 (m), −114.37 (s).

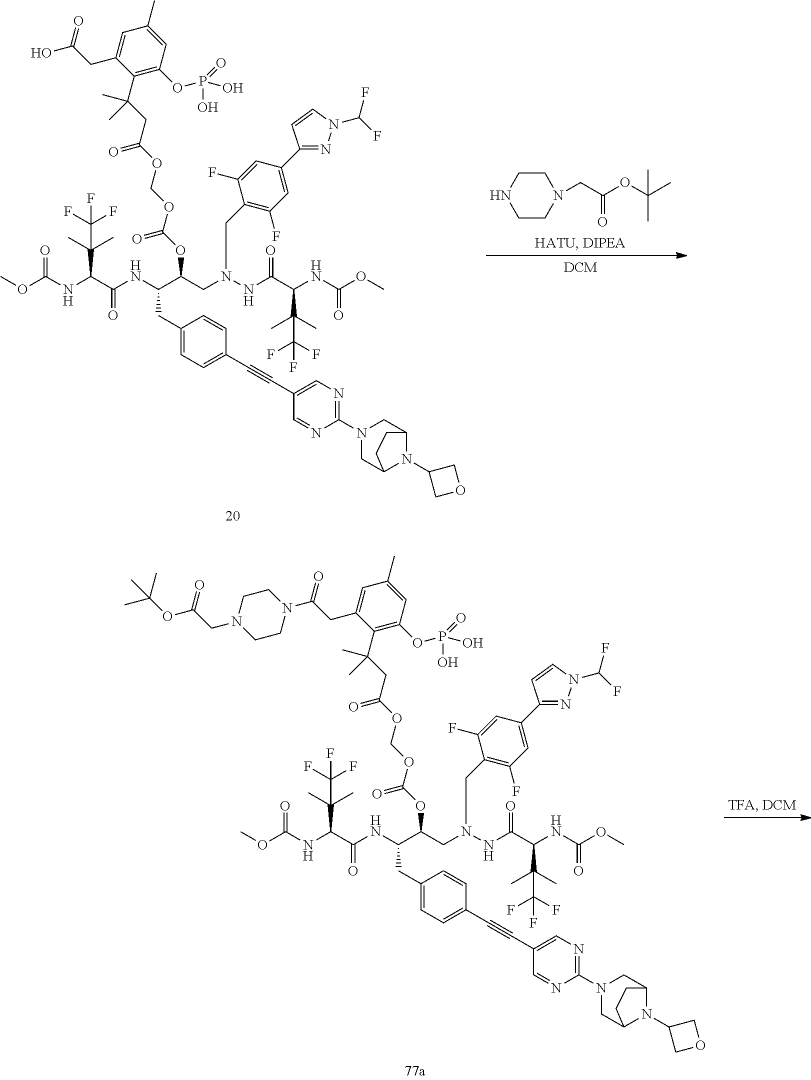 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F ; CC(C)(C)OC(=O)CN1CCNCC1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(CC(=O)OC(C)(C)C)CC2)cc(C)cc1OP(=O)(O)O)C(C)(C)C(F)(F)F]

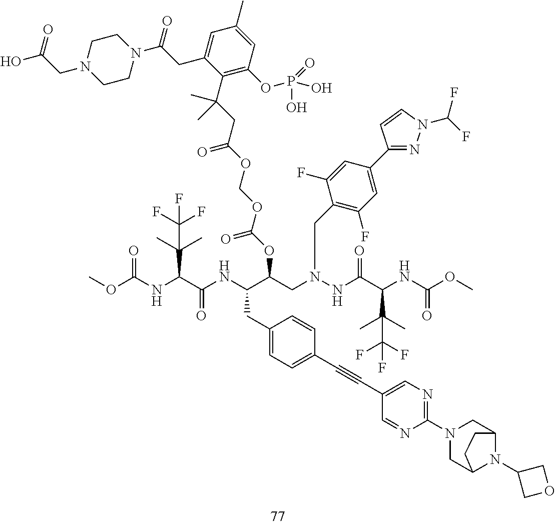 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(CC(=O)O)CC2)cc(C)cc1OP(=O)(O)O)C(C)(C)C(F)(F)F]

### Example 77

2-(4-(2-(2-((5S,10S)-8-(4-(1-(difluoromethyl)-1H-pyrazol-3-yl)-2,6-difluorobenzyl)-18-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12,16-tetraoxo-5-(1,1,1-trifluoro-2-methylpropan-2-yl)-2,11,13,15-tetraoxa-4,7,8-triazanonadecan-18-yl)-5-methyl-3-(phosphonooxy)phenyl)acetyl)piperazin-1-yl)acetic acid 77 was synthesized using the method described in Example 66, substituting tert-butyl 2-(piperazin-1-yl)acetate for di-tert-butyl L-glutamate hydrochloride and substituting 20 for 4. MS (m/z): 842.40 [M+2H]²⁺. ¹H NMR (400 MHz, Methanol-d4) δ 8.55 (s, 2H), 8.13 (d, J=2.8 Hz, 1H), 7.73-7.40 (m), 7.40-7.77 (m), 7.33-7.28 (m), 7.28-7.21 (m), 7.02 (d, J=10.0 Hz, 1H), 6.96 (d, J=2.8 Hz, 1H), 6.85 (d, J=10.0 Hz, 1H), 6.61-6.56 (m), 5.74 (d, J=5.9 Hz, 1H), 5.64 (d, J=5.9 Hz, 1H), 5.02-4.94 (m), 4.83-4.76 (m), 4.65-4.51 (m), 4.51-4.44 (m), 4.37-4.27 (m), 4.19-4.13 (m), 4.13-4.05 (m), 4.00-3.86 (m), 3.85-3.75 (m), 3.73-3.67 (m), 3.55-3.46 (m), 3.45-3.38 (m), 3.30-3.22 (m), 3.22-3.14 (m), 3.06-2.97 (m), 2.97-2.88 (m), 2.79-2.68 (m), 2.27-2.20 (m), 2.06-1.95 (m), 1.64-1.58 (m), 1.25-1.05 (in). ¹⁹F NMR (376 MHz, Methanol-d4) δ −77.41 (s), −77.62 (s), −96.84 (d, J=7.4 Hz), −97.00 (d, J=7.4 Hz), −114.63 (s).

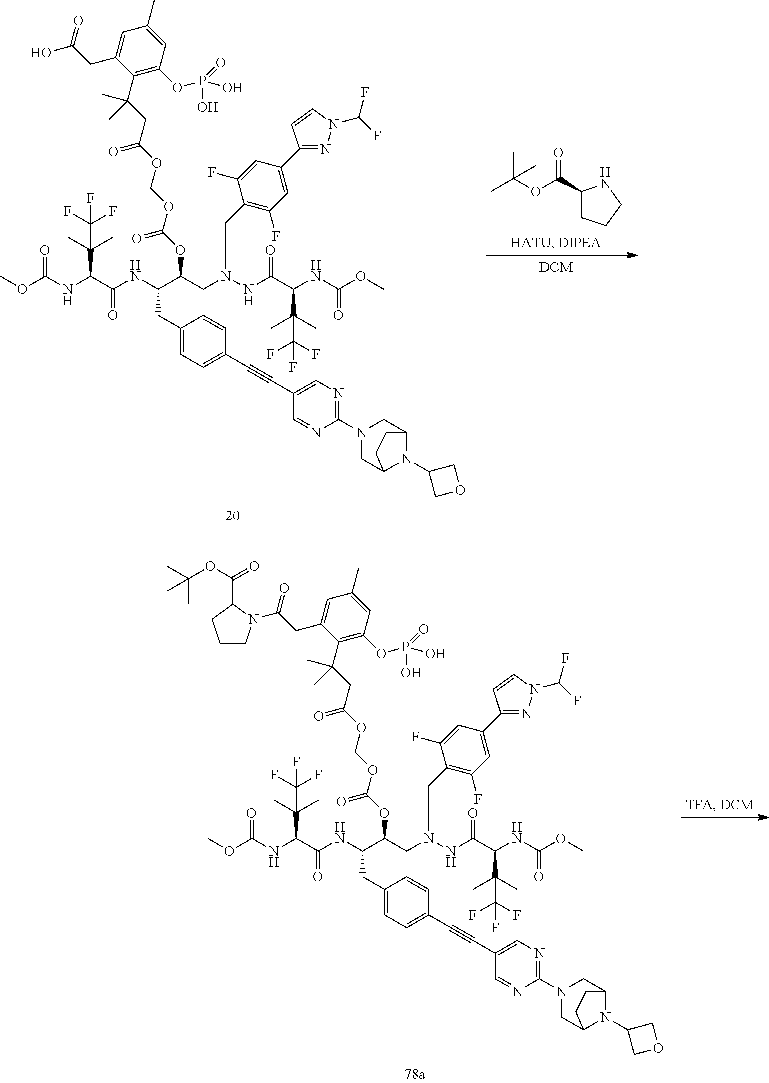 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F ; CC(C)(C)OC(=O)[C@@H]1CCCN1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCCC2C(=O)OC(C)(C)C)cc(C)cc1OP(=O)(O)O)C(C)(C)C(F)(F)F]

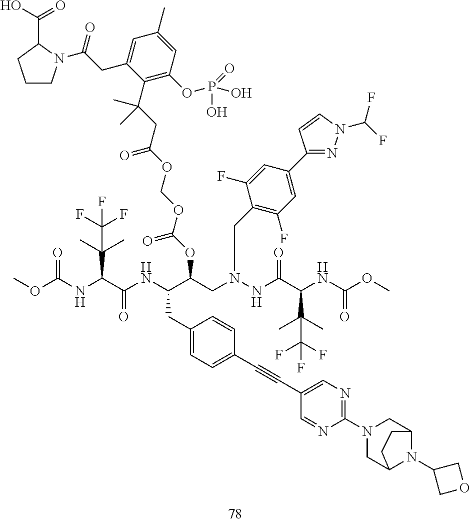 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCCC2C(=O)O)cc(C)cc1OP(=O)(O)O)C(C)(C)C(F)(F)F]

### Example 78

(2-(2-((3S,8S)-6-(4-(1-(difluoromethyl)-1H-pyrazol-3-yl)-2,6-difluorobenzyl)-1,1,1-trifluoro-3-((methoxycarbonyl)amino)-2,2,16-trimethyl-8-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-4,10,14-trioxo-9,11,13-trioxa-5,6-diazaheptadecan-16-yl)-5-methyl-3-(phosphonooxy)phenyl)acetyl)-L-proline 78 was synthesized using the method described in Example 66, substituting tert-butyl L-prolinate for di-tert-butyl L-glutamate hydrochloride and substituting 20 for 4. MS (m/z): 827.90 [M+2H]²⁺. ¹H NMR (400 MHz, Methanol-d4) δ 8.55 (s, 2H), 8.13 (d, J=2.7 Hz, 1H), 7.72-7.39 (m), 7.39-7.33 (m), 7.27-7.19 (m), 7.05-6.98 (m), 6.98-6.91 (m), 6.87-6.80 (m), 6.78-6.72 (m), 6.62-6.58 (m), 5.76 (d, J=6.0 Hz, 1H), 5.62 (dd, J=6.0, 1.7 Hz, 1H), 5.02-4.94 (m), 4.86-4.76 (m), 4.54-4.51 (m), 4.51-4.41 (m), 4.37-4.26 (m), 4.19-4.14 (m), 4.14-4.08 (m), 4.08-3.94 (m), 3.73-3.67 (m), 3.64-3.56 (m), 3.54-3.46 (m), 3.30-3.19 (m), 3.19-3.06 (m), 3.04-2.86 (m), 2.80-2.68 (m), 2.31-2.21 (m), 2.09-1.95 (m), 1.66-1.58 (m), 1.25-1.12 (m), 1.07 (s, 3H). ¹⁹F NMR (376 MHz, Methanol-d4) δ −77.42 (s), −77.67 (s), −96.84 (d, J=5.2 Hz), −97.00 (d, J=5.2 Hz), −114.81 (s).

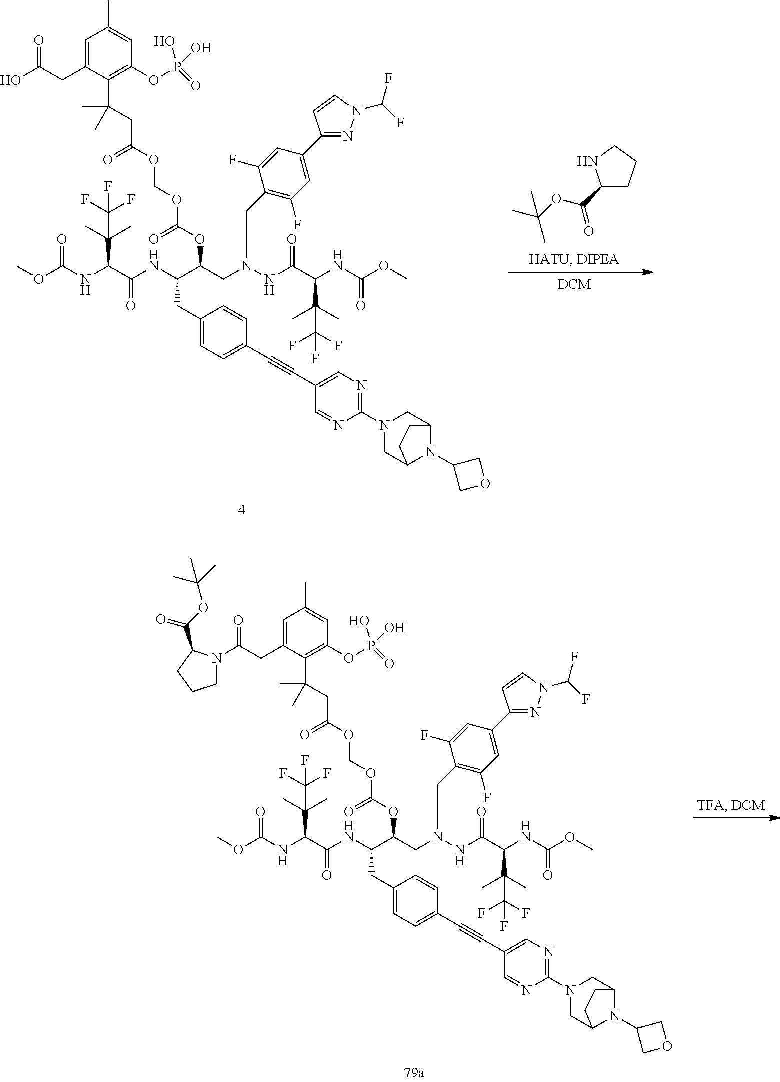 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F ; CC(C)(C)OC(=O)[C@@H]1CCCN1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCC[C@H]2C(=O)OC(C)(C)C)cc(C)cc1OP(=O)(O)O)C(C)(C)C(F)(F)F]

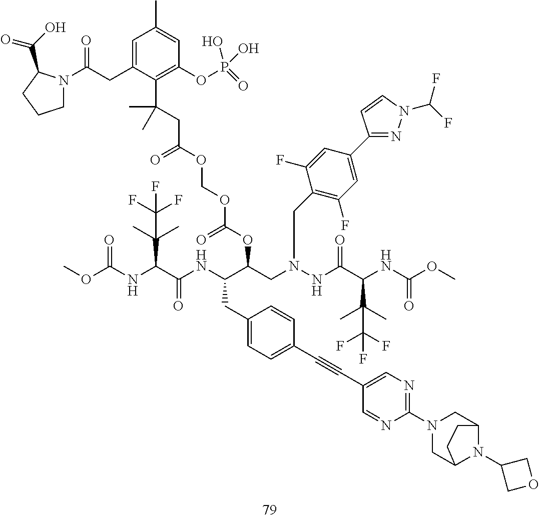 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCC[C@H]2C(=O)O)cc(C)cc1OP(=O)(O)O)C(C)(C)C(F)(F)F]

### Example 79

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-proline 79 was synthesized using the method described in Example 66, substituting tert-butyl L-prolinate for di-tert-butyl L-glutamate hydrochloride. MS (m/z): 790.90 [M+2H]²⁺. ¹H NMR (400 MHz, Methanol-d4) δ 8.55 (m), 8.13 (d, J=2.8 Hz, 1H), 7.72-7.39 (m), 7.35-7.22 (m), 7.14-7.06 (m), 7.00-6.88 (m), 6.78-6.69 (m), 5.02-4.94 (m), 4.81-4.74 (m), 4.65-4.46 (m), 4.43-4.34 (m), 4.34-4.12 (m), 4.00-3.89 (m), 3.81-3.66 (m), 3.66-3.59 (m), 3.59-3.44 (m), 3.03-2.84 (m), 2.81-2.71 (m), 2.38-2.20 (m), 2.10-1.93 (m), 1.80-1.69 (m), 1.67-1.59 (m), 1.25-1.12 (m), 1.09-0.96 (m). ¹⁹F NMR (376 MHz, Methanol-d4) δ −77.11-−77.18 (m), −77.73-−77.80 (m), −96.86 (d, J=6.3 Hz), −97.02 (d, J=6.4 Hz), −114.42 (s).

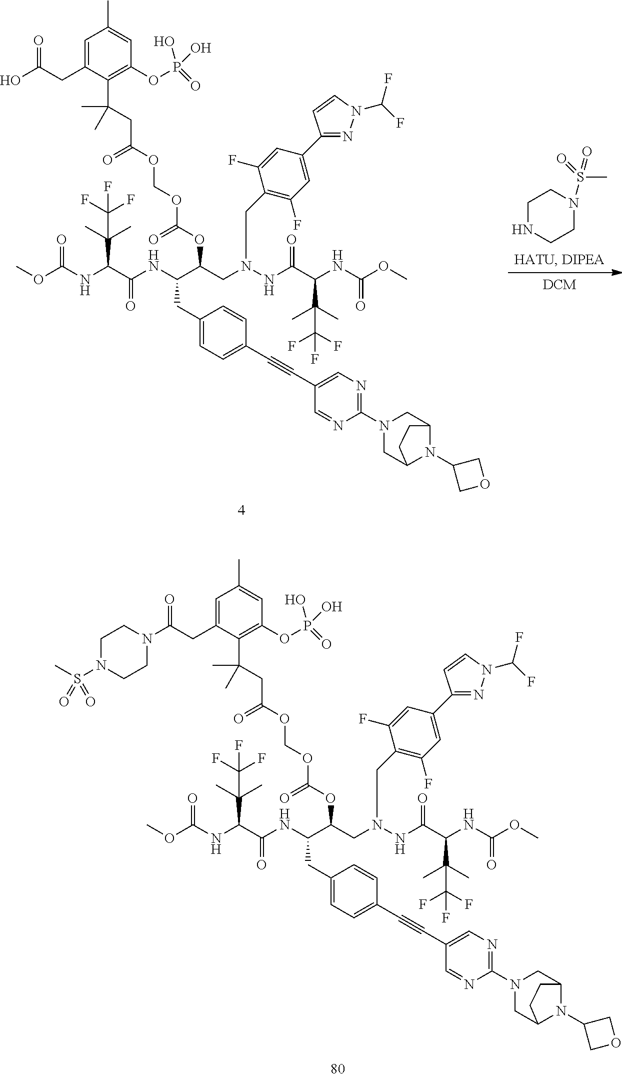

### Example 80

(5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-(4-(methylsulfonyl)piperazin-1-yl)-2-oxoethyl)-6-(phosphonooxy)phenyl)butanoate 80 was synthesized using the method described in Example 66, substituting 1-(methylsulfonyl)piperazine for di-tert-butyl L-glutamate hydrochloride. MS (m/z): 815.40 [M+2H]²⁺. ¹H NMR (400 MHz, Methanol-d4) δ 8.55 (s, 2H), 8.13 (d, J=2.7 Hz, 1H), 7.72-7.39 (m), 7.35-7.25 (m), 7.16-7.09 (m), 6.99-6.91 (m), 6.61-6.53 (m), 5.02-4.94 (m), 4.84-4.76 (m), 4.61-4.47 (m), 4.40-4.26 (m), 4.25-4.12 (m), 4.05-3.89 (m), 3.89-3.77 (m), 3.77-3.63 (m), 3.62-3.48 m), 3.47-3.37 (m), 3.29-3.20 (m), 3.20-3.11 (m), 3.02-2.88 (m), 2.85-2.75 (m), 2.35-2.19 (m), 2.13-1.97 (m), 1.74 (s, 3H), 1.63 (s, 3H), 1.25-1.14 (m), 1.03 (s, 3H). ¹⁹F NMR (376 MHz, Methanol-d4) δ −77.18 (s), −77.69 (s), −96.87 (d, J=15.9 Hz), −97.00 (d, J=15.8 Hz), −114.50.

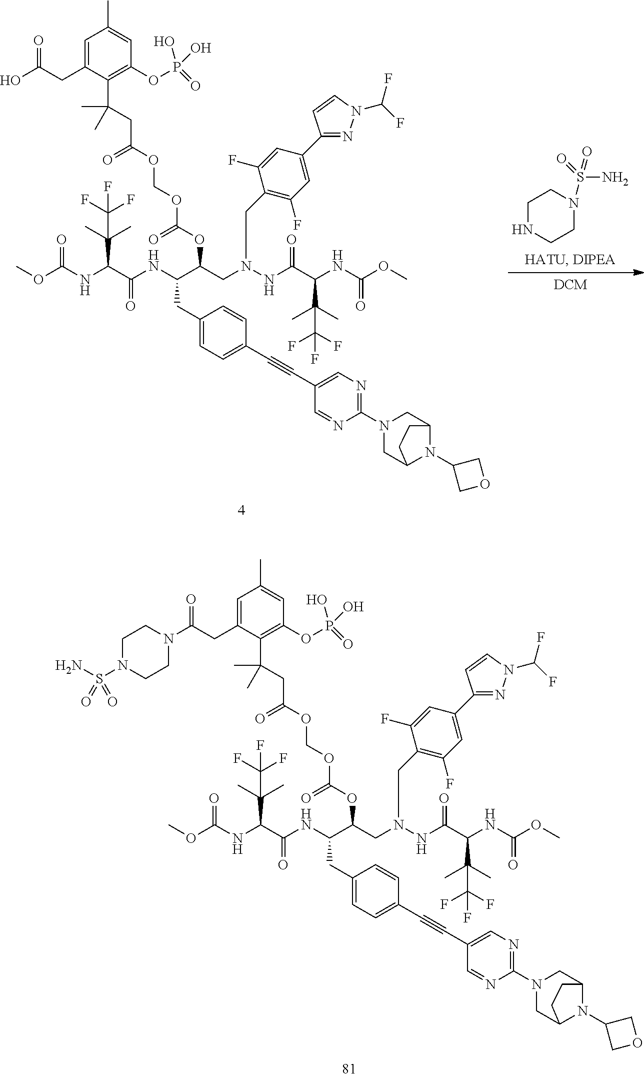 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F ; NS(=O)(=O)N1CCNCC1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(S(N)(=O)=O)CC2)cc(C)cc1OP(=O)(O)O)C(C)(C)C(F)(F)F]

### Example 81

(5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-oxo-2-(4-sulfamoylpiperazin-1-yl)ethyl)-6-(phosphonooxy)phenyl)butanoate 81 was synthesized using the method described in Example 66, substituting piperazine-1-sulfonamide for di-tert-butyl L-glutamate hydrochloride. MS (m/z): 815.90 [M+2H]²⁺. ¹H NMR (400 MHz, Methanol-d4) δ 8.55 (s, 2H), 8.13 (d, J=2.7 Hz, 1H), 7.72-7.38 (m), 7.34-7.22 (m), 7.15-7.06 (m), 6.99-6.90 (m), 6.63-6.45 (m), 5.02-4.94 (m), 4.81-4.71 (m), 4.55-4.53 (m), 4.40-4.25 (m), 4.25-4.08 (m), 3.98-3.84 (m), 3.83-3.61 (m), 3.59-3.47 (m), 3.22-3.04 (m), 3.01-2.87 (m), 2.80-2.70 (m), 2.36-2.17 (m), 2.15-1.95 (m), 1.77-1.68 (m), 1.63 (s, 3H), 1.25-1.10 (m), 1.01 (s, 3H). ¹⁹F NMR (376 MHz, Methanol-d4) δ −77.18 (s), −77.74 (s), −96.87 (d, J=14.0 Hz), −97.03 (d, J=14.0 Hz), −114.52 (s).

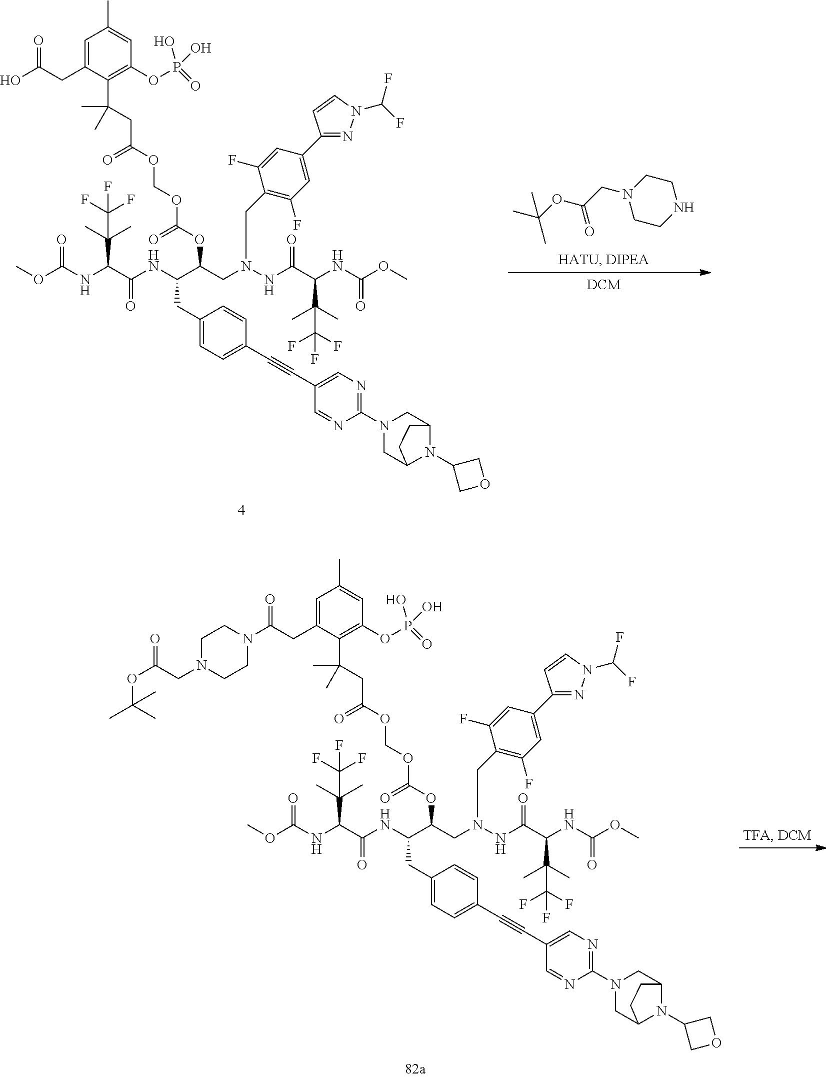 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F ; CC(C)(C)OC(=O)CN1CCNCC1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(CC(=O)OC(C)(C)C)CC2)cc(C)cc1OP(=O)(O)O)C(C)(C)C(F)(F)F]

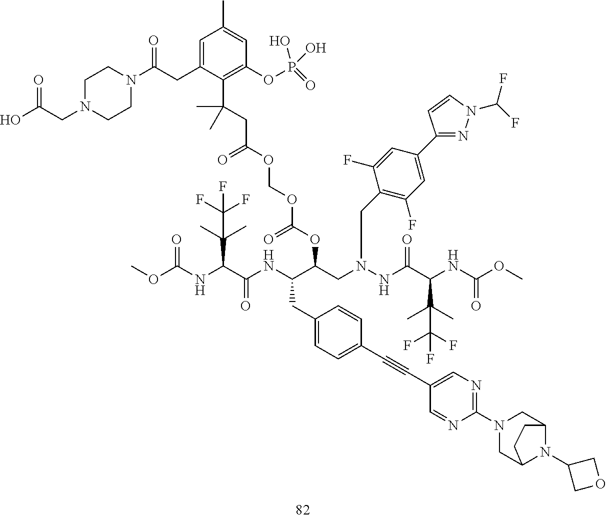 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(CC(=O)O)CC2)cc(C)cc1OP(=O)(O)O)C(C)(C)C(F)(F)F]

### Example 82

2-(4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperazin-1-yl)acetic acid 82 was synthesized using the method described in Example 66, substituting tert-butyl 2-(piperazin-1-yl)acetate for di-tert-butyl L-glutamate hydrochloride. MS (m/z): 805.40 [M+2H]²⁺. ¹H NMR (400 MHz, Methanol-d4) δ 8.55 (s, 2H), 8.13 (d, J=2.7 Hz, 1H), 7.73-7.39 (m), 7.36-7.27 (m), 7.17-7.07 (m), 6.99-6.91 (m), 6.76-6.68 (m), 6.60-6.53 (m), 5.01-4.93 (m), 4.83-4.75 (m), 4.60-4.45 (m), 4.43-4.26 (m), 4.22-4.09 (m), 4.00-3.86 (m), 3.80-3.65 (m), 3.65-3.52 (m), 3.52-3.40 (m), 3.08-2.99 (m), 2.99-2.84 (m), 2.76-2.67 (m), 2.37-2.19 (m), 2.17-2.09 (m), 2.09-1.92 (m), 1.78-1.68 (m), 1.64 (s, 3H), 1.28-1.14 (m), 1.01 (s, 3H). ¹⁹F NMR (376 MHz, Methanol-d4) δ −77.15 (s), −77.74 (s), −96.81-−96.90 (m), −97.00-−97.07 (m), −114.50 (s).

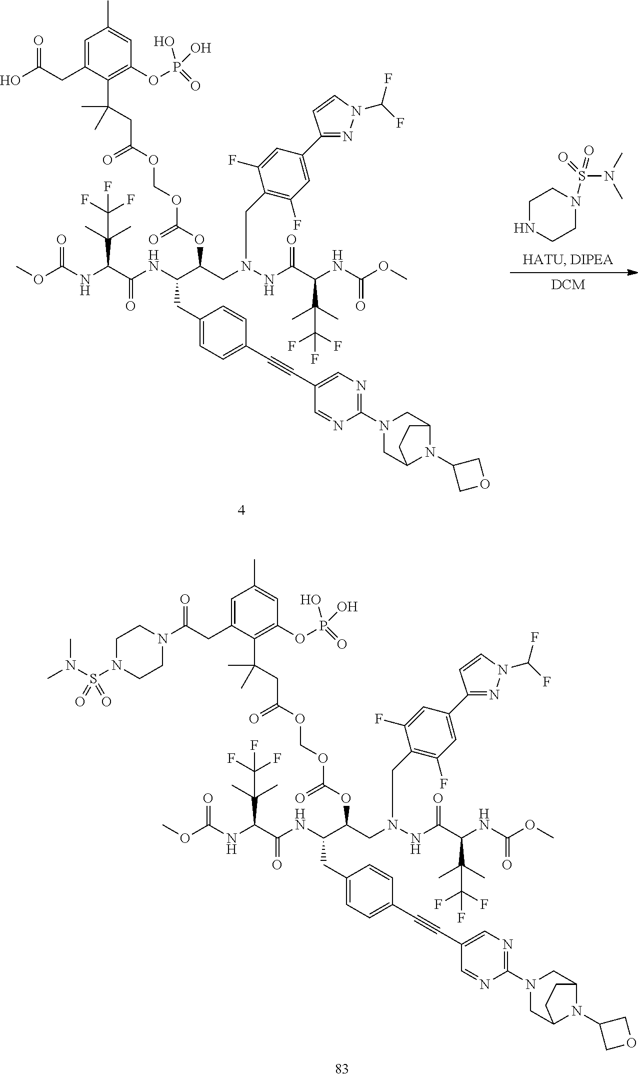 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)O)cc(C)cc1OP(=O)(O)O)C(C)(C)C(F)(F)F ; CN(C)S(=O)(=O)N1CCNCC1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)OCOC(=O)CC(C)(C)c1c(CC(=O)N2CCN(S(=O)(=O)N(C)C)CC2)cc(C)cc1OP(=O)(O)O)C(C)(C)C(F)(F)F]

### Example 83

(5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-(2-(2-(4-(N,N-dimethylsulfamoyl)piperazin-1-yl)-2-oxoethyl)-4-methyl-6-(phosphonooxy)phenyl)-3-methylbutanoate 83 was synthesized using the method described in Example 66, substituting N,N-dimethylpiperazine-1-sulfonamide for di-tert-butyl L-glutamate hydrochloride. MS (m/z): 829.90 [M+2H]²⁺. ¹H NMR (400 MHz, Methanol-d4) δ 8.55 (s, 2H), 8.13 (d, J=2.8 Hz, 1H), 7.72-7.39 (m), 7.34-7.25 (m), 7.15-7.08 (m), 6.99-6.90 (m), 6.73-6.67 (m), 6.61-6.55 (m), 5.03-4.94 (m), 4.81-4.75 (m), 4.61-4.46 (m), 4.31-4.26 (m), 4.25-4.11 (m), 3.98-3.89 (m), 3.81-3.66 (m), 3.66-3.60 (m), 3.60-3.47 (m), 3.29-3.18 (m), 3.02-2.91 (m), 2.83 (s, 6H), 2.80-2.71 (m), 2.34-2.20 (m), 2.14-1.96 (m), 1.73 (s, 3H), 1.63 (s, 3H), 1.25-1.16 (m), 1.02 (s, 3H). ¹⁹F NMR (376 MHz, Methanol-d4) δ−77.18 (s), −77.72 (s), −96.84-−96.90 (m), −97.01-−99.05 (m), −114.52 (s).

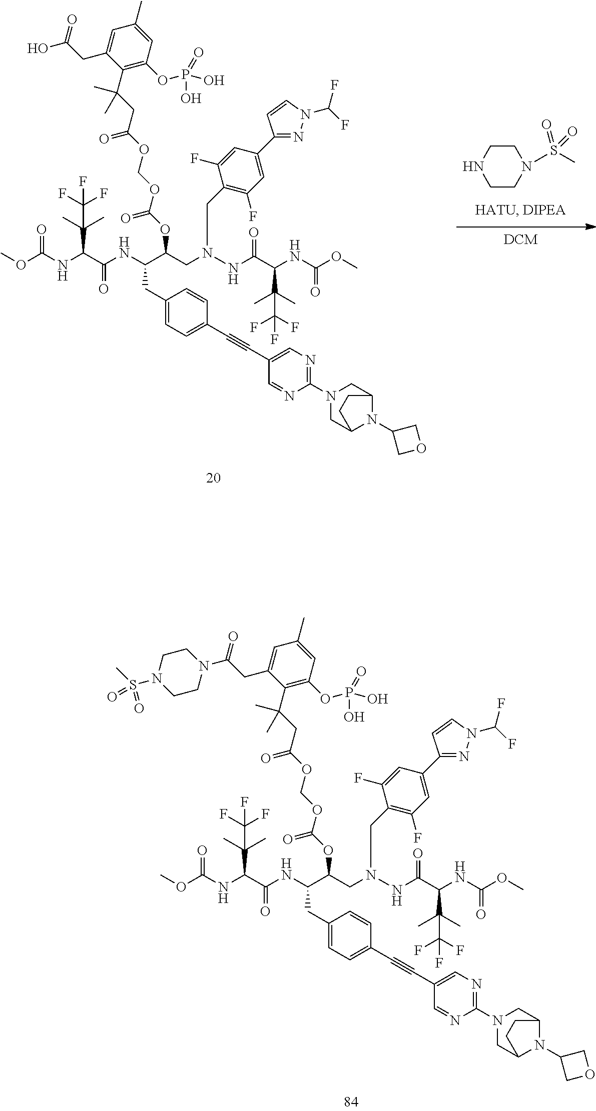

### Example 84

(5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-methyl-3-(4-methyl-2-(2-(4-(methylsulfonyl)piperazin-1-yl)-2-oxoethyl)-6-(phosphonooxy)phenyl)butanoate 84 was synthesized using the method described in Example 66, substituting 1-(methylsulfonyl)piperazine for di-tert-butyl L-glutamate hydrochloride and substituting 20 for 4. MS (m/z): 852.80 [M+2H]²⁺. ¹H NMR (400 MHz, Methanol-d4) δ 8.55 (s, 2H), 8.13 (d, J=2.7 Hz, 1H), 7.73-7.40 (m), 7.39-7.36 (m), 7.29-7.21 (m), 7.04-6.93 (m), 6.88-6.79 (m), 6.64-6.58 (m), 5.78 (d, J=6.0 Hz, 1H), 5.61 (d, J=5.9 Hz, 1H), 5.02-4.94 (m), 4.85-4.76 (m), 4.64-4.54 (m), 4.54-4.44 (m), 4.37-4.26 (m), 4.20-4.13 (m), 4.13-4.03 (m), 3.79-3.65 (m), 3.63-3.55 (m), 3.54-3.45 (m), 3.30-3.20 (m), 3.19-3.12 (m), 3.03-2.94 (m), 2.94-2.86 (m), 2.86-2.83 (m), 2.79-2.68 (m), 2.29-2.19 (m), 2.07-1.96 (m), 1.63-1.57 (m), 1.24-1.13 (m), 1.07 (s, 3H). ¹⁹F NMR (376 MHz, Methanol-d4) δ −77.38 (s), −77.63 (s), −96.84 (d, J=11.4 Hz), −97.00 (d, J=11.4 Hz), −114.77 (s).

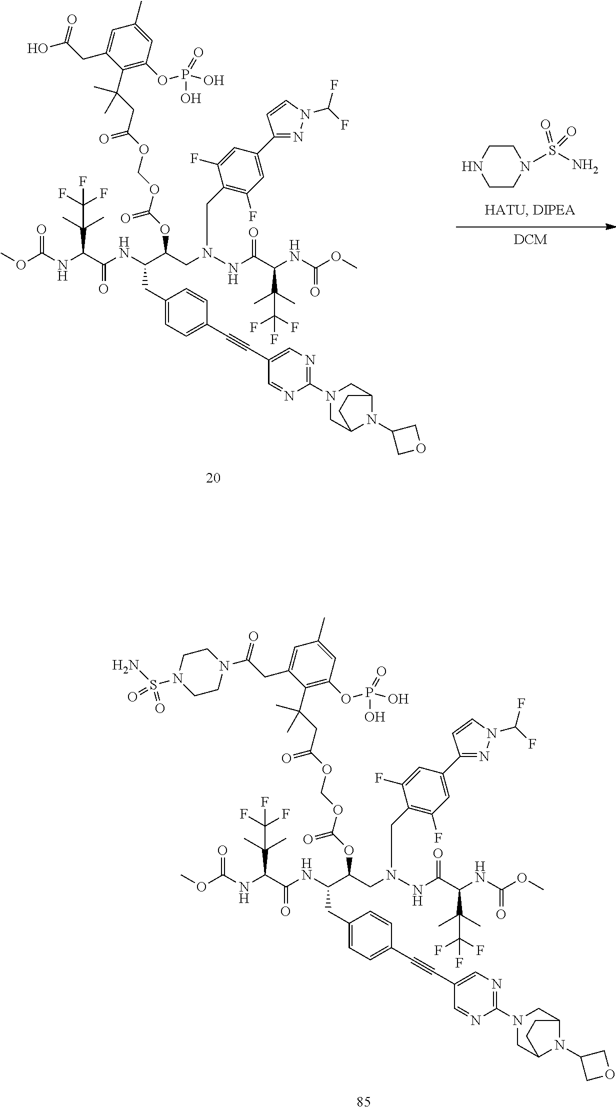

### Example 85

(5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1—((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-methyl-3-(4-methyl-2-(2-oxo-2-(4-sulfamoylpiperazin-1-yl)ethyl)-6-(phosphonooxy)phenyl)butanoate 85 was synthesized using the method described in Example 66, substituting piperazine-1-sulfonamide for di-tert-butyl L-glutamate hydrochloride and substituting 20 for 4. MS (m/z): 853.20 [M+2H]²⁺. ¹H NMR (400 MHz, Methanol-d4) δ 8.55 (s, 2H), 8.13 (d, J=2.7 Hz, 1H), 7.73-7.39 (m), 7.39-7.34 (m), 7.28-7.21 (m), 7.04-6.93 (m), 6.87-6.79 (m), 6.64-6.58 (m), 5.77 (d, J=5.9 Hz, 1H), 5.61 (d, J=5.9 Hz, 1H), 5.03-4.94 (m), 4.86-4.76 (m), 4.64-4.56 (m), 4.52-4.46 (m), 4.38-4.26 (m), 4.19-4.13 (m), 4.13-4.03 (m), 3.84-3.74 (m), 3.74-3.66 (m), 3.62-3.54 (m), 3.54-3.46 (m), 3.30-3.21 (m), 3.20-3.14 (m), 3.14-2.95 (m), 2.95-2.86 (m), 2.79-2.68 (m), 2.28-2.20 (m), 2.06-1.96 (m), 1.64-1.57 (m), 1.25-1.12 (m), 1.07 (s, 3H). ¹⁹F NMR (376 MHz, Methanol-d4) δ −77.40 (s), −77.65 (s), −96.84 (d, J=11.4 Hz), −97.00 (d, J=11.4 Hz), −114.65 (s).

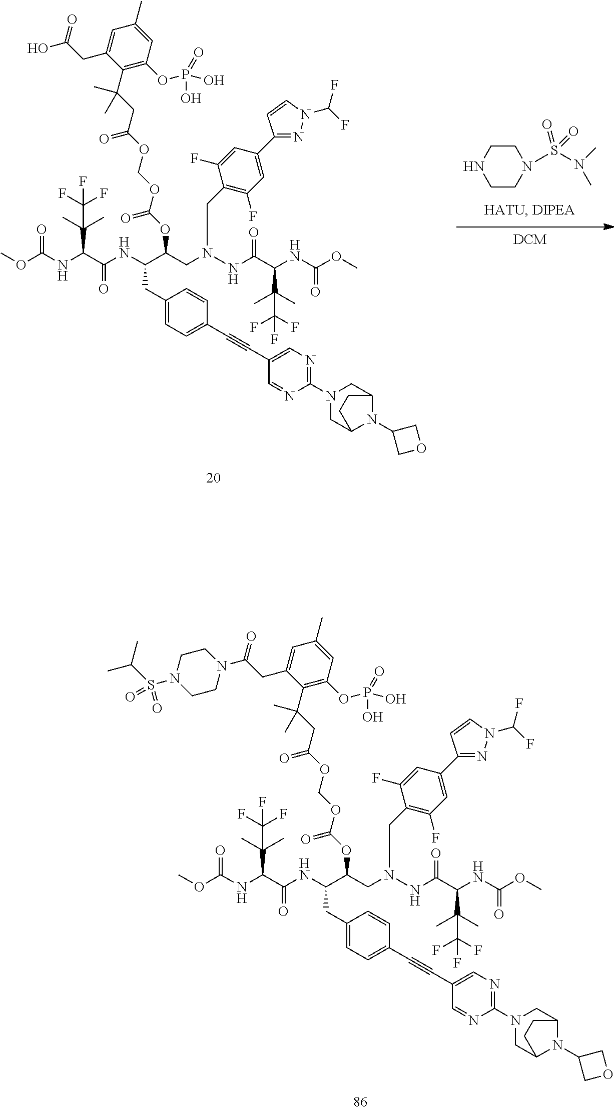

### Example 86

(5S,10S)-8-(4-(1-(difluoromethyl)-1H-pyrazol-3-yl)-2,6-difluorobenzyl)-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11,13-trioxa-4,7,8-triazatetradecan-14-yl 3-(2-(2-(4-(N,N-dimethylsulfamoyl)piperazin-1-yl)-2-oxoethyl)-4-methyl-6-(phosphonooxy)phenyl)-3-methylbutanoate 86 was synthesized using the method described in Example 66, substituting N,N-dimethylpiperazine-1-sulfonamide for di-tert-butyl L-glutamate hydrochloride and substituting 20 for 4. MS (m/z): 866.80 [M+2H]²⁺. ¹H NMR (400 MHz, Methanol-d4) δ 8.55 (s, 2H), 8.13 (d, J=2.7 Hz, 1H), 7.73-7.39 (m), 7.39-7.33 (m), 7.30-7.20 (m), 7.04-6.93 (m), 6.88-6.79 (m), 6.64-6.58 (m), 5.77 (d, J=6.0 Hz, 1H), 5.61 (d, J=5.9 Hz, 1H), 5.02-4.93 (m), 4.84-4.73 (m), 4.64-4.56 (m), 4.56-4.45 (m), 4.38-4.26 (m), 4.19-4.13 (m), 4.13-4.04 (m), 3.75-3.64 (m, 3.58-3.45 (m), 3.27-3.10 (m), 3.03-2.95 (m), 2.95-2.85 (m), 2.85-2.81 (m), 2.79-2.69 (m), 2.27-2.20 (m), 2.03-1.98 (m), 1.64-1.57 (m), 1.25-1.12 (m), 1.06 (s, 3H). ¹⁹F NMR (376 MHz, Methanol-d4) δ −77.38 (s), −77.64 (s), −96.83 (d, J=11.3 Hz), −96.99 (d, J=11.4 Hz), −114.63 (s).

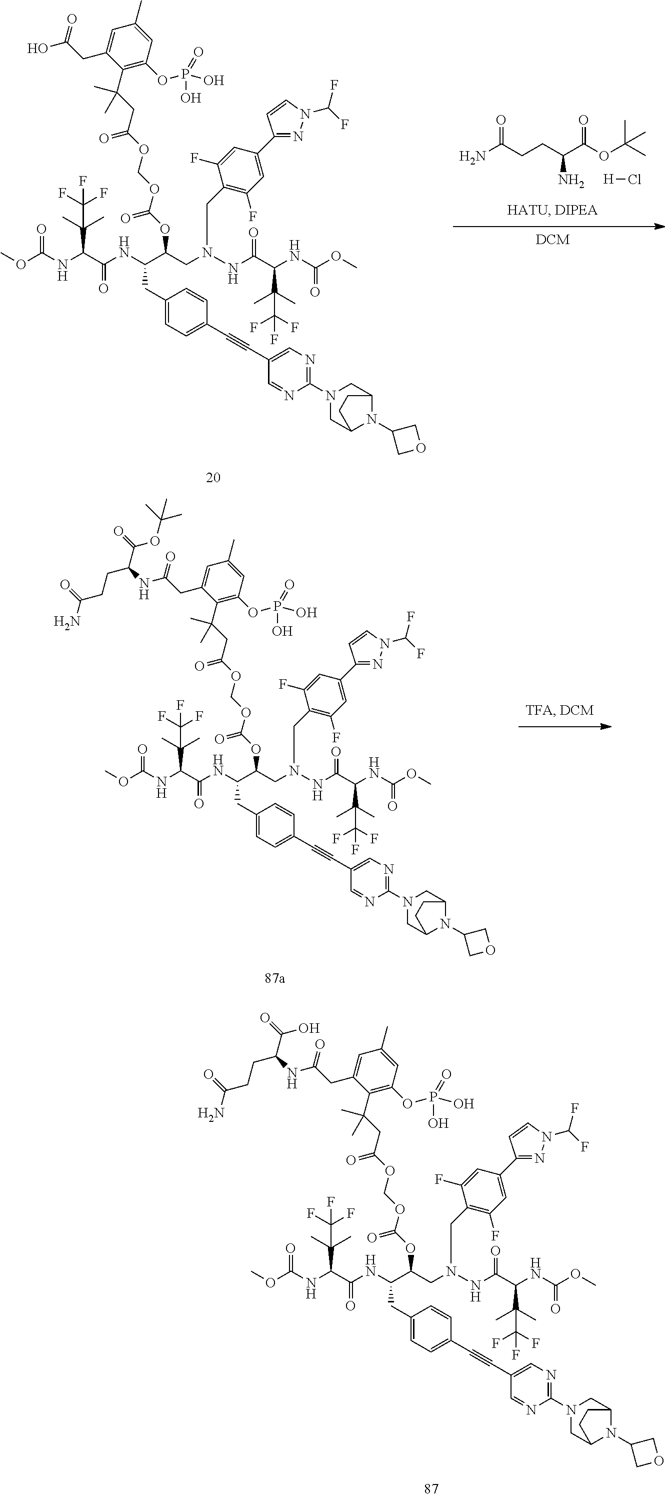

### Example 87

(2-(2-((3S,8S)-6-(4-(1-(difluoromethyl)-1H-pyrazol-3-yl)-2,6-difluorobenzyl)-1,1,1-trifluoro-3-((methoxycarbonyl)amino)-2,2,16-trimethyl-8-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-4,10,14-trioxo-9,11,13-trioxa-5,6-diazaheptadecan-16-yl)-5-methyl-3-(phosphonooxy)phenyl)acetyl)-L-glutamine 87 was synthesized using the method described in Example 66, substituting tert-butyl L-glutaminate hydrochloride for di-tert-butyl L-glutamate hydrochloride and substituting 20 for 4. MS (m/z): 843.80 [M+2H]²⁺. ¹H NMR (400 MHz, Methanol-d4) δ 8.55 (s, 2H), 8.12 (d, J=2.7 Hz, 1H), 7.73-7.39 (m), 7.39-7.33 (m), 7.30-7.21 (m), 7.06-6.98 (m), 6.98-6.92 (m), 6.85-6.77 (m), 5.74 (d, J=5.9 Hz, 1H), 5.65 (d, J=5.9 Hz, 1H), 5.03-4.94 (m), 4.85-4.76 (m), 4.66-4.57 (m), 4.57-4.43 (m), 4.38-4.26 (m), 4.20-4.13 (m), 4.13-4.05 (m), 4.05-3.88 (m), 3.74-3.66 (m), 3.54-3.46 (m), 3.30-3.08 (m), 3.04-2.86 (m), 2.80-2.69 (m), 2.38-2.27 (m), 2.27-2.12 (m), 2.08-1.92 (m), 1.68-1.60 (m), 1.25-1.11 (m), 1.07 (s, 3H). ¹⁹F NMR (376 MHz, Methanol-d4) δ −77.39 (s), −77.68 (s), −96.84 (d, J=6.3 Hz), −97.00 (d, J=6.4 Hz), −114.61 (s).

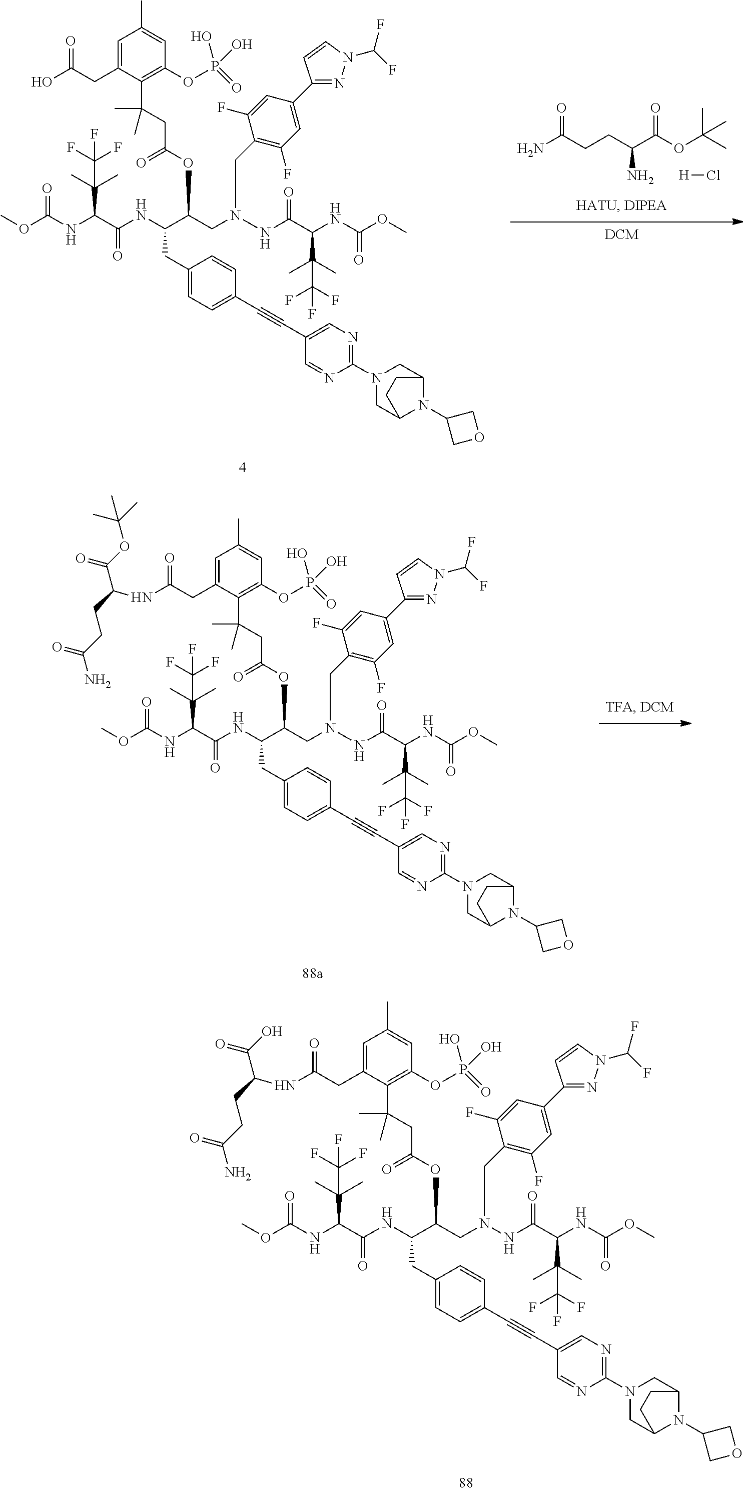

### Example 88

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3, 8-diazabicyclo[13.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-glutamine 88 was synthesized using the method described in Example 66, substituting tert-butyl L-glutaminate hydrochloride for di-tert-butyl L-glutamate hydrochloride. MS (m/z): 806.80 [M+2H]²⁺. ¹H NMR (400 MHz, Methanol-d4) δ 8.54 (s, 2H), 8.12 (d, J=2.7 Hz, 1H), 7.73-7.39 (m), 7.36-7.23 (m), 7.16-7.08 (m), 6.99-6.91 (m), 6.80-6.74 (m), 5.03-4.94 (m), 4.85-4.74 (m), 4.60-4.47 (m), 4.41-4.28 (m), 4.26-4.13 (m), 3.99-3.91 (m), 3.83-3.65 (m), 3.56-3.40 (m), 3.01-2.91 (m), 2.88-2.72 (m), 2.40-2.28 (m), 2.28-2.16 (m), 2.16-2.07 (m), 2.07-1.95 (m), 1.82-1.76 (m), 1.69-1.62 (m), 1.24-1.15 (m), 1.02 (s, 3H). ¹⁹F NMR (376 MHz, Methanol-d4) δ −77.20 (s), −77.80 (s), −96.87 (d, J=6.4 Hz), −97.03 (d, J=6.5 Hz), −114.49 (s).

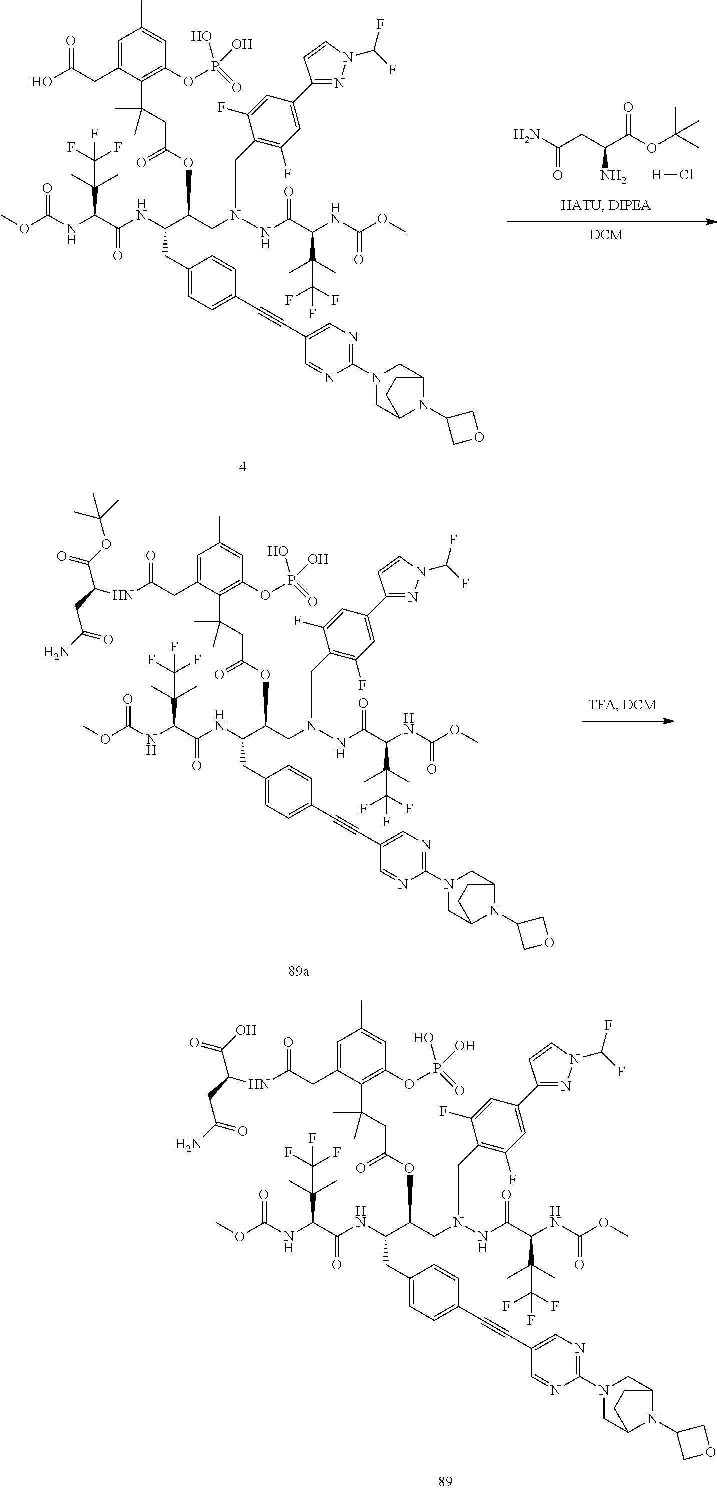

### Example 89

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-asparagine 89 was synthesized using the method described in Example 66, substituting tert-butyl L-asparaginate hydrochloride for di-tert-butyl L-glutamate hydrochloride. MS (m/z): 799.80 [M+2H]²⁺. ¹H NMR (400 MHz, Methanol-d4) δ 8.54 (s, 2H), 8.12 (d, J=2.8 Hz, 1H), 7.72-7.38 (m), 7.35-7.24 (m), 7.34-7.24 (m), 7.15-7.07 (m), 6.99-6.88 (m), 6.78-6.70 (m), 5.03-4.94 (m), 4.85-4.74 (m), 4.60-4.44 (m), 4.40-4.27 (m), 4.27-4.12 (m), 4.00-3.91 (m), 3.81-3.63 (m), 3.56-3.39 (m), 3.00-2.91 (m), 2.88-2.71 (m), 2.37-2.18 (m), 2.15-1.94 (m), 1.79 (s, 3H), 1.65 (s, 3H), 1.24-1.15 (m), 1.03 (s, 3H). ¹⁹F NMR (376 MHz, Methanol-d4) δ −77.21 (s), −77.80 (s), −96.87 (d, J=7.1 Hz), −97.03 (d, J=7.2 Hz), −114.42 (s).

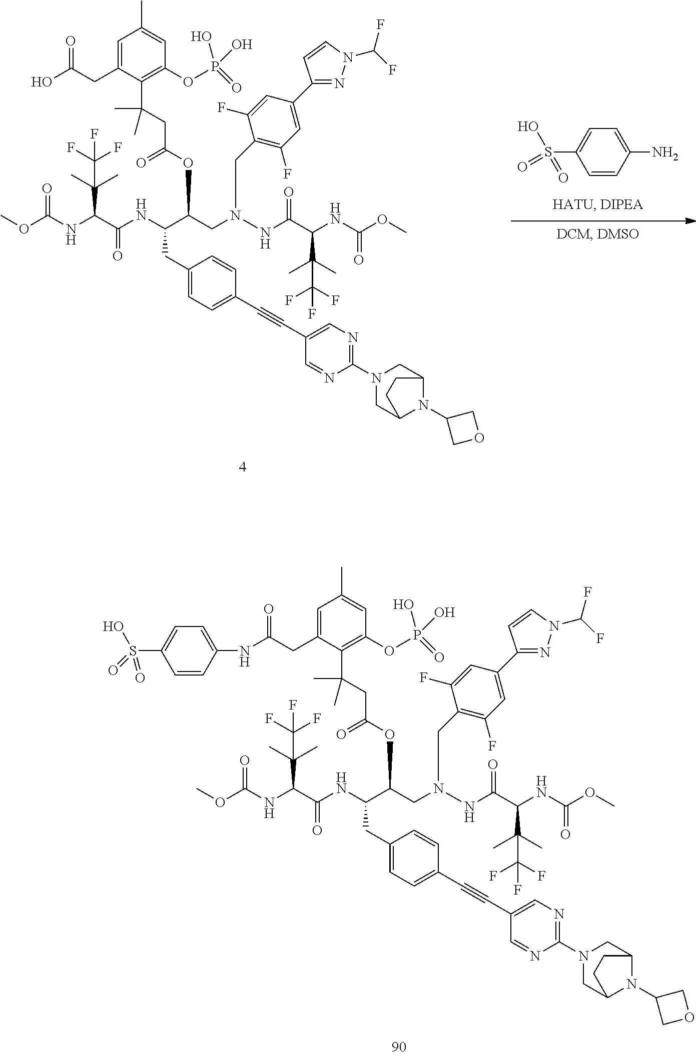

### Example 90

To a suspension of 4 (0.022 mmol) in DCM (1 mL) was added a solution of 4-aminobenzenesulfonic acid (0.055 mmol), N,N-diisopropylethylamine (0.142 mmol) in DMSO (0.75 mL) followed by HATU (0.077 mmol). The reaction was stirred at room temperature for 5 hours, then concentrated. The residue obtained was diluted with DMSO and purified by reverse-phase HPLC (eluent: water/MeCN 0.1% TFA) to afford 4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzenesulfonic acid (90). MS (m/z): 820.20 [M+2H]²⁺. ¹H NMR (400 MHz, Methanol-d4) δ 8.53 (s), 8.11 (d, J=2.7 Hz, 1H), 7.81-7.73 (m), 7.72-7.60 (m), 7.58-7.37 (m), 7.34-7.24 (m), 7.12-7.04 (m), 6.97-6.92 (m), 6.80-6.73 (m), 5.03-4.94 (m), 4.84-4.74 (m), 4.54-4.43 (m), 4.43-4.26 (m), 4.26-4.13 (m), 4.02-3.88 (m), 3.80-3.65 (m), 3.53-3.36 (m), 3.054-2.94 (m), 2.94-2.73 (m), 2.37-2.27 (m), 2.27-2.18 (m), 2.18-2.08 (m), 2.05-1.95 (m), 1.80 (s, 3H), 1.66 (s, 3H), 1.26-1.14 (m), 0.99 (s, 3H). ¹⁹F NMR (376 MHz, Methanol-d4) δ −77.18 (s), −77.78 (s), −96.87 (d, J=9.7 Hz), −97.02 (d, J=9.6 Hz), −114.54 (s).

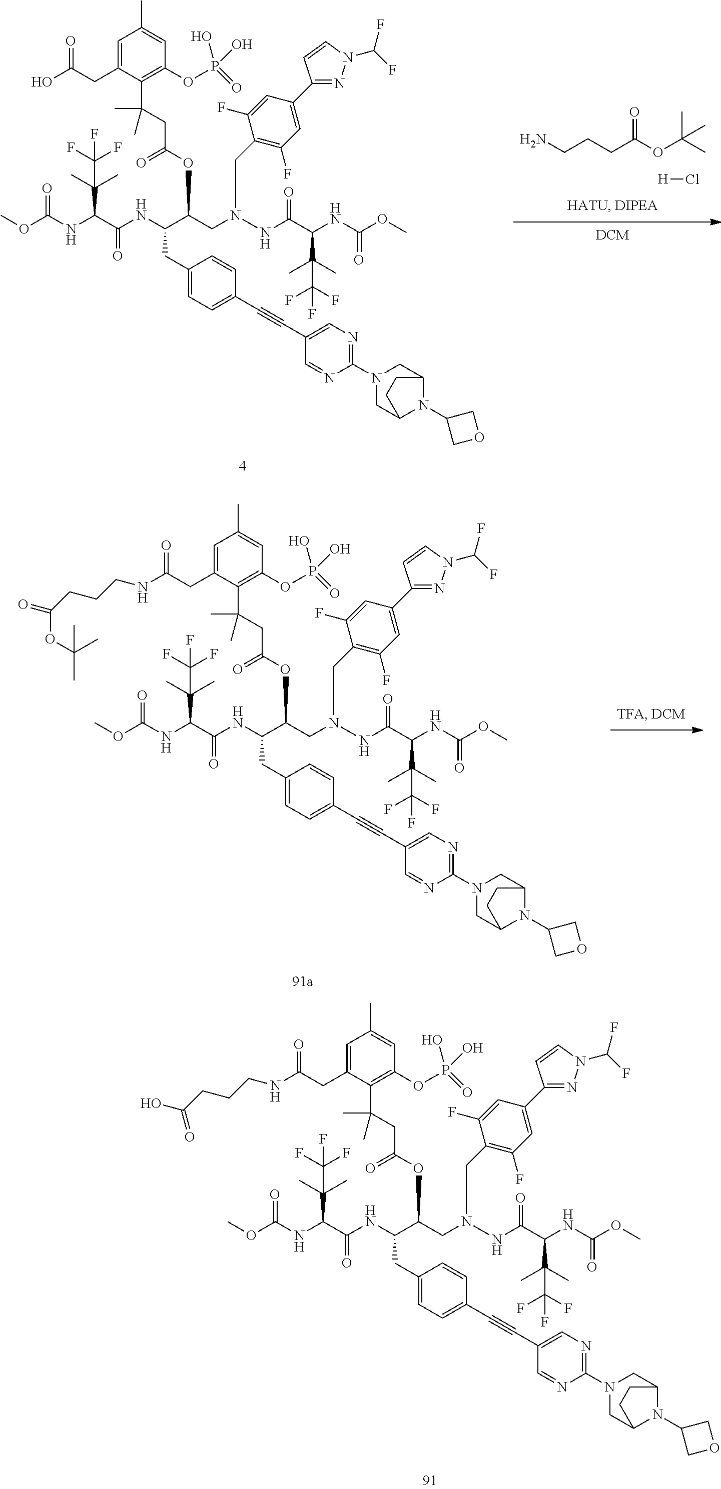

### Example 91

4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)butanoic acid 91 was synthesized using the method described in Example 66, substituting tert-butyl 4-aminobutanoate hydrochloride for di-tert-butyl L-glutamate hydrochloride. MS (m/z): 785.20 [M+2H]²⁺. ¹H NMR (400 MHz, Methanol-d4) 8.54 (s, 2H), 8.12 (d, J=2.8 Hz, 1H), 7.72-7.38 (m), 7.36-7.24 (m), 7.15-7.08 (m), 6.99-6.88 (m), 6.78-6.68 (m), 5.03-4.94 (m), 4.82-4.73 (m), 4.60-4.44 (m), 4.42-4.34 (m), 4.34-4.26 (m), 4.25-4.06 (m), 3.99-3.89 (m), 3.80-3.64 (m), 3.60-3.38 (m), 3.30-3.23 (m), 3.06-3.95 (m), 2.94-2.83 (m), 2.83-2.73 (m), 2.38-2.29 (m), 2.28-2.20 (m), 2.19-2.06 (m), 2.06-1.95 (m), 1.86-1.73 (m), 1.64 (s, 3H), 1.25-1.14 (m), 1.00 (s, 3H). ¹⁹F NMR (376 MHz, Methanol-d4) δ −77.18 (s), −77.79 (s), −96.87 (d, J=7.4 Hz), −97.03 (d, J=7.3 Hz), −114.49 (s).

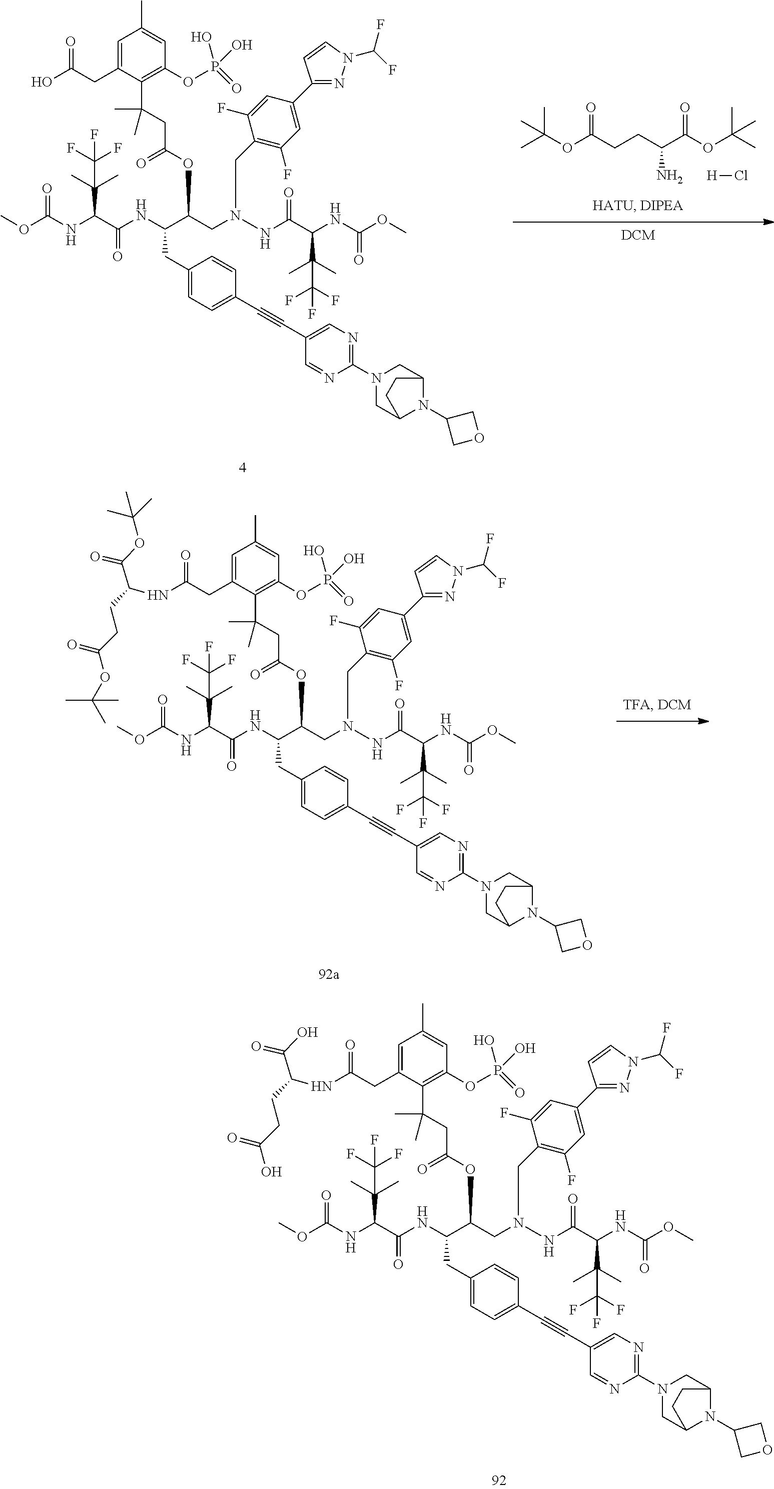

### Example 92

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-D-glutamic acid 92 was synthesized using the method described in Example 66, substituting di-tert-butyl D-glutamate hydrochloride for di-tert-butyl L-glutamate hydrochloride. MS (m/z): 807.20 [M+2H]²⁺. ¹H NMR (400 MHz, Methanol-d4) δ 8.54 (s, 2H), 8.12 (d, J=2.8 Hz, 1H), 7.72-7.38 (m), 7.36-7.22 (m), 7.16-7.08 (m), 6.98-6.88 (m), 6.78-6.70 (m), 5.03-4.94 (m), 4.84-4.75 (m), 4.60-4.48 (m), 4.41-4.33 (m), 4.33-4.26 (m), 4.25-4.12 (m), 3.99-3.91 (m), 3.84-3.76 (m), 3.76-3.65 (m), 3.57-3.47 (m), 3.46-3.37 (m), 3.04-2.95 (m), 2.93-2.85 (m), 2.84-2.74 (m), 2.47-2.38 (m), 2.38-2.28 (m), 2.28-2.17 (m), 2.17-2.05 (m), 2.05-1.93 (m), 1.80 (s, 3H), 1.65 (s, 3H), 1.25-1.13 (m), 1.00 (s, 3H). ¹⁹F NMR (376 MHz, Methanol-d4) δ −77.16 (s), −77.80 (s), −96.86 (d, J=6.9 Hz), −97.02 (d, J=6.9 Hz), −114.46 (s).

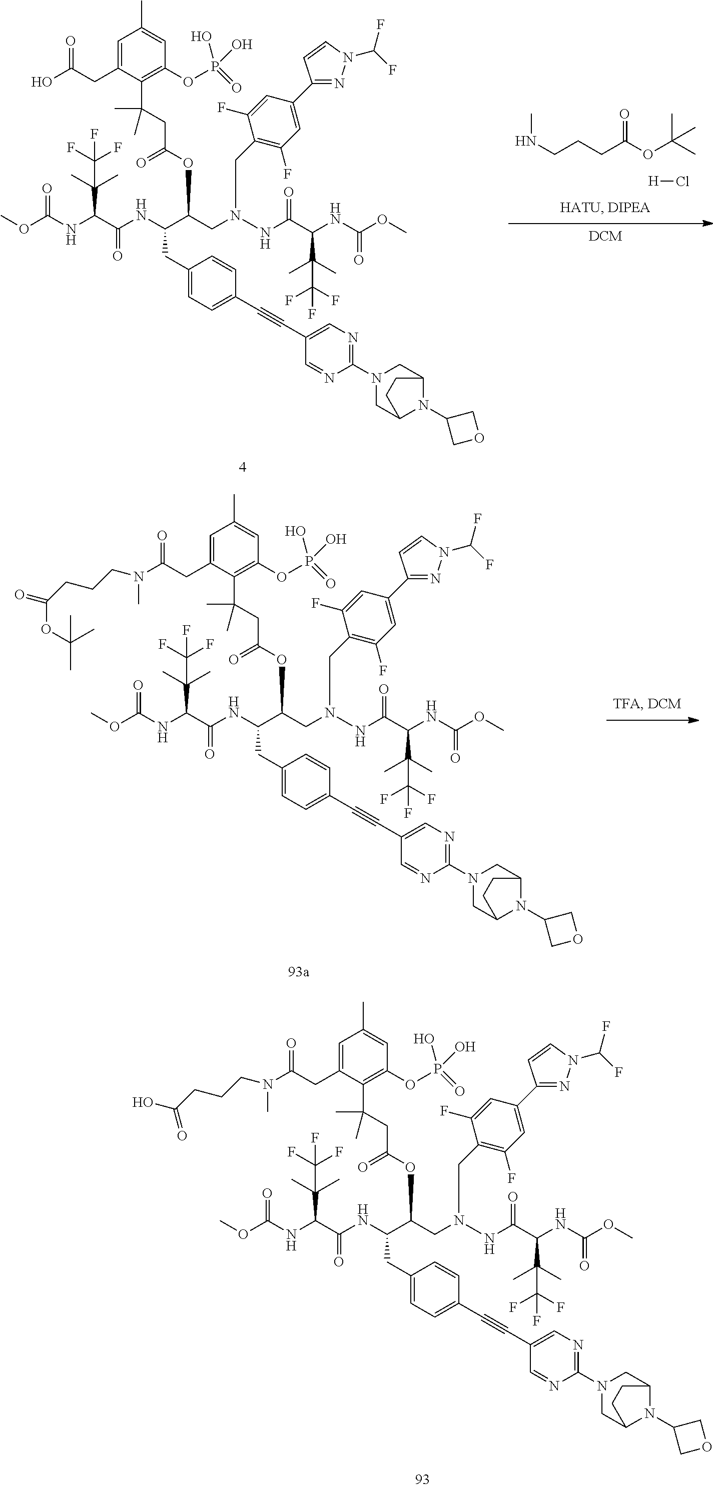

### Example 93

4-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)-N-methylacetamido)butanoic acid 93 was synthesized using the method described in Example 66, substituting tert-butyl 4-(methylamino)butanoate hydrochloride for di-tert-butyl L-glutamate hydrochloride. MS (m/z): 792.30 [M+2H]²⁺. ¹H NMR (400 MHz, Methanol-d4) δ 8.54 (s, 2H), 8.12 (dd, J=2.8, 1.5 Hz, 1H), 7.73-7.39 (m), 7.34-7.23 (m), 7.17-7.06 (m), 7.01-6.90 (m), 6.63-6.55 (m), 5.03-4.94 (m), 4.85-4.76 (m), 4.60-4.50 (m), 4.40-4.25 (m), 4.25-4.13 (m), 3.98-3.84 (m), 3.84-3.65 (m), 3.59-3.39 (m), 3.13-3.07 (m), 3.02-2.93 (m), 2.90-2.69 (m), 2.77-2.74 (m), 2.43-2.20 (m), 2.15-1.96 (m), 1.96-1.83 (m), 1.76-1.69 (m), 1.66-1.60 (m), 1.24-1.14 (m), 1.05-0.97 (m). ¹⁹F NMR (376 MHz, Methanol-d4) δ −77.16 (s), −77.77 (s), −96.77 (d, J=9.1 Hz), −97.01 (d, J=9.1 Hz), −114.44 (s).

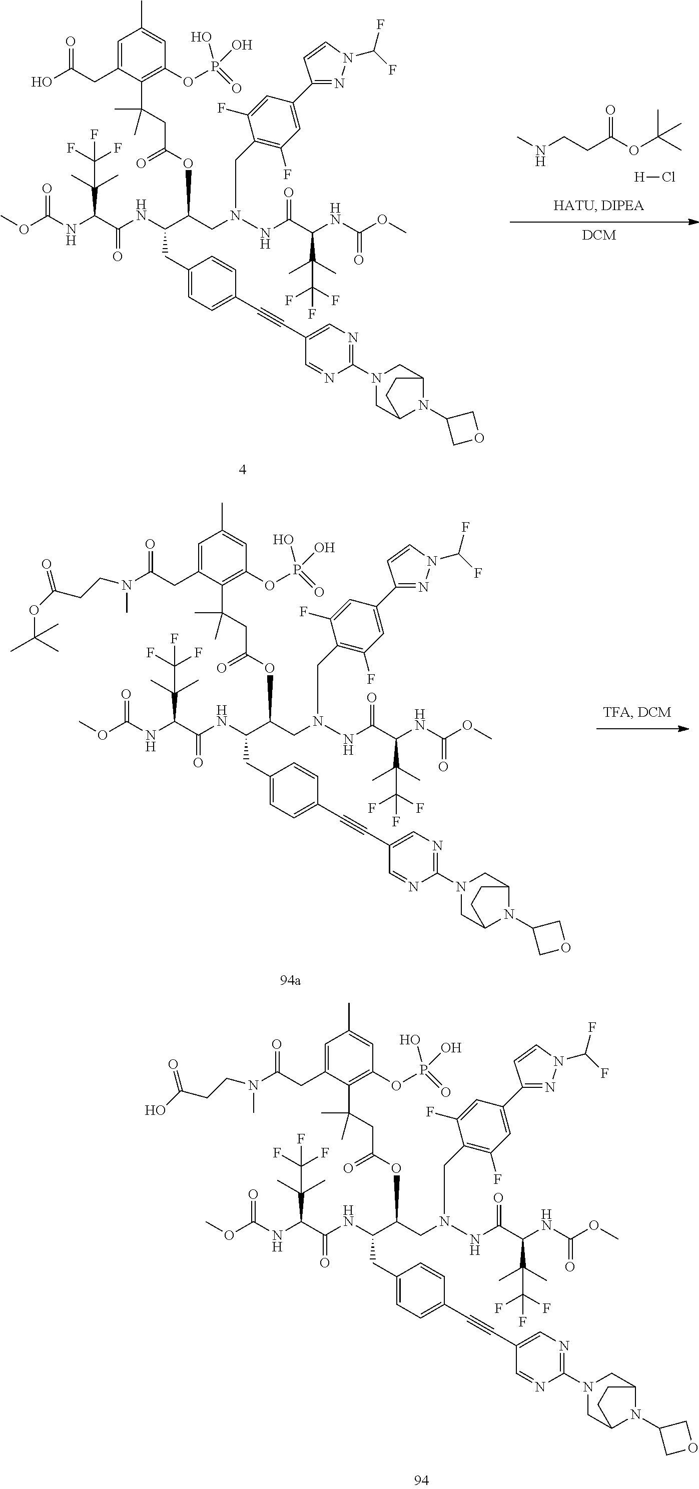

### Example 94

3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)-N-methylacetamido)propanoic acid 94 was synthesized using the method described in Example 66, substituting tert-butyl 3-(methylamino)propanoate hydrochloride for di-tert-butyl L-glutamate hydrochloride. MS (m/z): 785.20 [M+2H]²⁺. ¹H NMR (400 MHz, Methanol-d4) δ 8.54 (s, 2H), 8.12 (d, J=2.7 Hz, 1H), 7.72-7.38 (m), 7.34-7.22 (m), 7.16-7.07 (m), 6.99-6.89 (m), 6.75-6.67 (m), 6.61-6.53 (m), 5.03-4.94 (m), 4.82-4.75 (m), 4.61-4.48 (m), 4.44-4.26 (m), 4.26-4.12 (m), 4.03-3.90 (m), 3.83-3.58 (m), 3.58-3.47 (m), 3.47-3.39 (m), 3.18-3.12 (m), 3.03-2.93 (m), 2.93-2.82 (m), 2.82-2.72 (m), 2.72-2.64 (m), 2.64-2.57 (m), 2.36-2.18 (m), 2.14-1.95 (m), 1.78-1.68 (m), 1.66-1.38 (m), 1.26-1.13 (m), 1.05-0.97 (m). ¹⁹F NMR (376 MHz, Methanol-d4) δ −77.17 (s), −77.80-−77.81 (m), −96.87 (d, J=8.9 Hz), −97.03 (d, J=8.9 Hz), −114.44 (s).

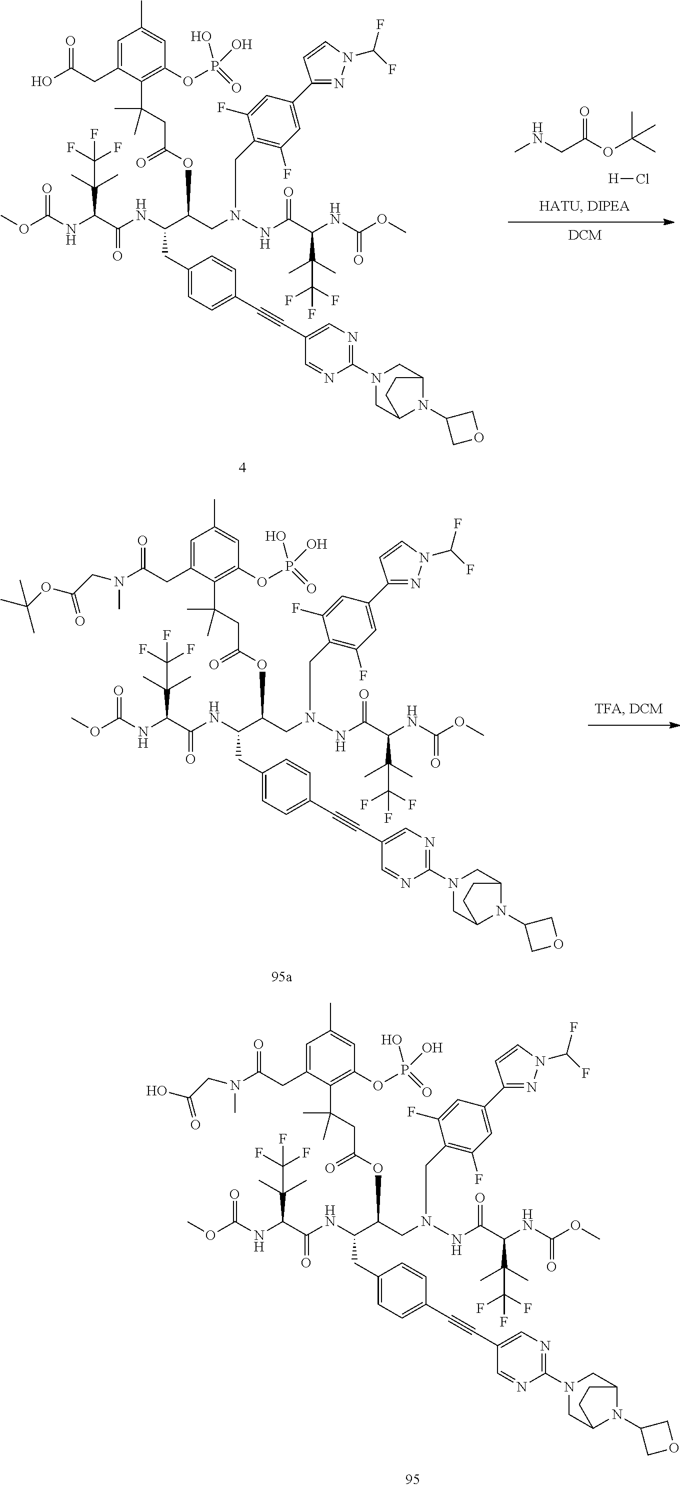

### Example 95

N-(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-N-methylglycine 95 was synthesized using the method described in Example 66, substituting tert-butyl methylglycinate for di-tert-butyl L-glutamate hydrochloride. MS (m/z): 778.20 [M+2H]²⁺. ¹H NMR (400 MHz, Methanol-d4) δ 8.54 (s, 2H), 8.12 (d, J=2.7 Hz, 1H), 7.55 (s, 1H), 7.73-7.39 (m), 7.34-7.23 (m), 7.15-7.07 (m), 6.99-6.87 (m), 6.75-6.66 (m), 6.58-6.47 (m), 5.03-4.94 (m), 4.83-4.72 (m), 4.61-4.47 (m), 4.43-4.25 (m), 4.25-4.10 (m), 4.01-3.86 (m), 3.86-3.71 (m), 3.71-3.64 (m), 3.59-3.40 (m), 3.20-3.12 (m), 3.06-2.92 (m), 2.91-2.70 (m), 2.35-2.19 (m), 2.13-1.95 (m), 1.81-1.69 (m), 1.66-1.60 (m), 1.26-1.14 (m), 1.06-0.97 (m). ¹⁹F NMR (376 MHz, Methanol-d4) δ−77.18 (s), −77.88 (s), −96.87 (d, J=6.7 Hz), −97.03 (d, J=6.6 Hz), −114.44 (s).

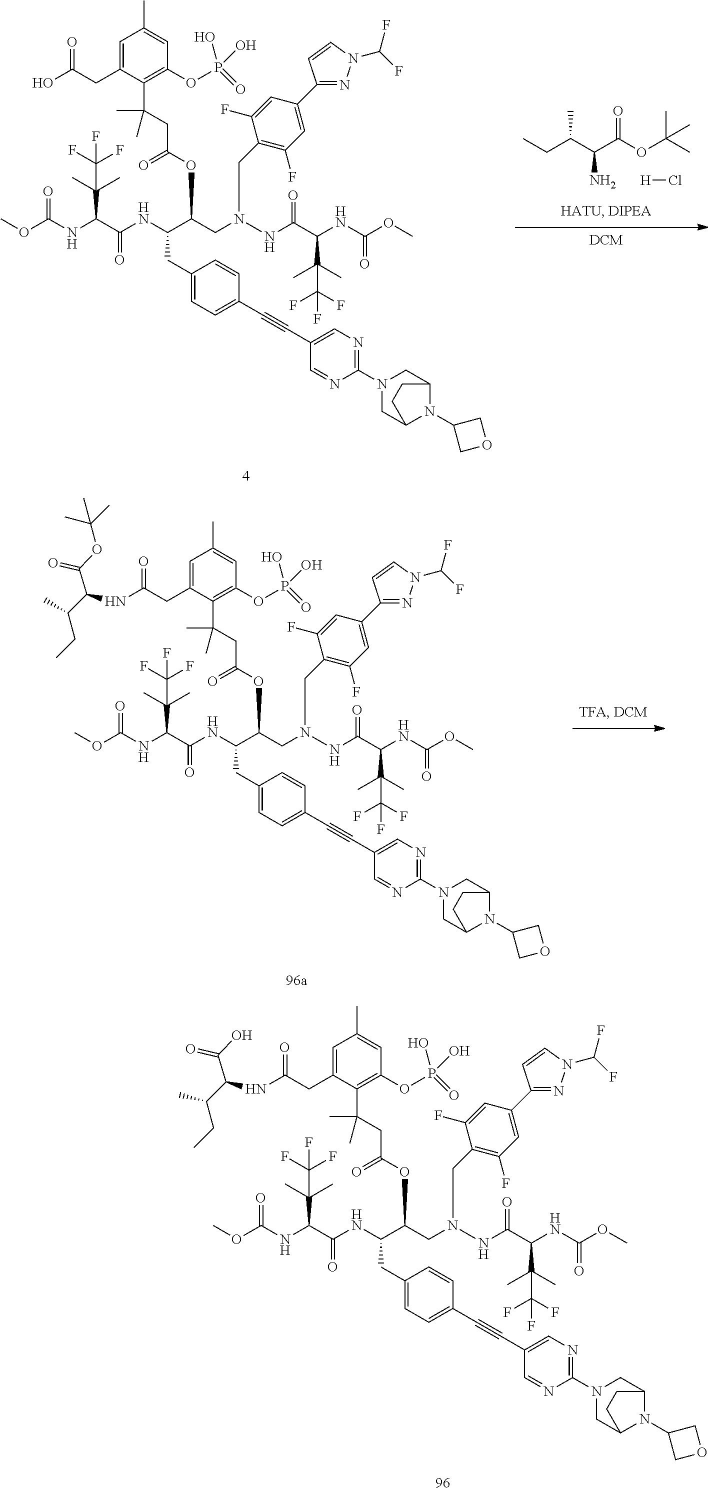

### Example 96

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-isoleucine 96 was synthesized using the method described in Example 66, substituting tert-butyl L-isoleucinate hydrochloride for di-tert-butyl L-glutamate hydrochloride. MS (m/z): 799.20 [M+2H]²⁺. ¹H NMR (400 MHz, Methanol-d4) δ 8.55 (s, 2H), 8.12 (d, J=2.7 Hz, 1H), 7.73-7.38 (m), 7.36-7.25 (m), 7.15-7.07 (m), 6.99-6.89 (m), 6.77-6.67 (m), 5.03-4.94 (m), 4.84-4.75 (m), 4.61-4.45 (m), 4.40-4.25 (m), 4.25-4.11 (m), 4.00-3.91 (m), 3.80-3.65 (m), 3.63-3.41 (m), 3.03-2.92 (m), 2.87-2.74 (m), 2.35-2.18 (m), 2.15-1.98 (m), 1.98-1.87 (m), 1.79 (s, 3H), 1.65 (s, 3H), 1.56-1.45 (m), 1.27-1.12 (m), 1.05-0.89 (m). ¹⁹F NMR (376 MHz, Methanol-d4) δ −77.16 (s), −77.85 (s), −96.87 (d, J=7.1 Hz), −97.03 (d, J=7.1 Hz), −114.44 (s).

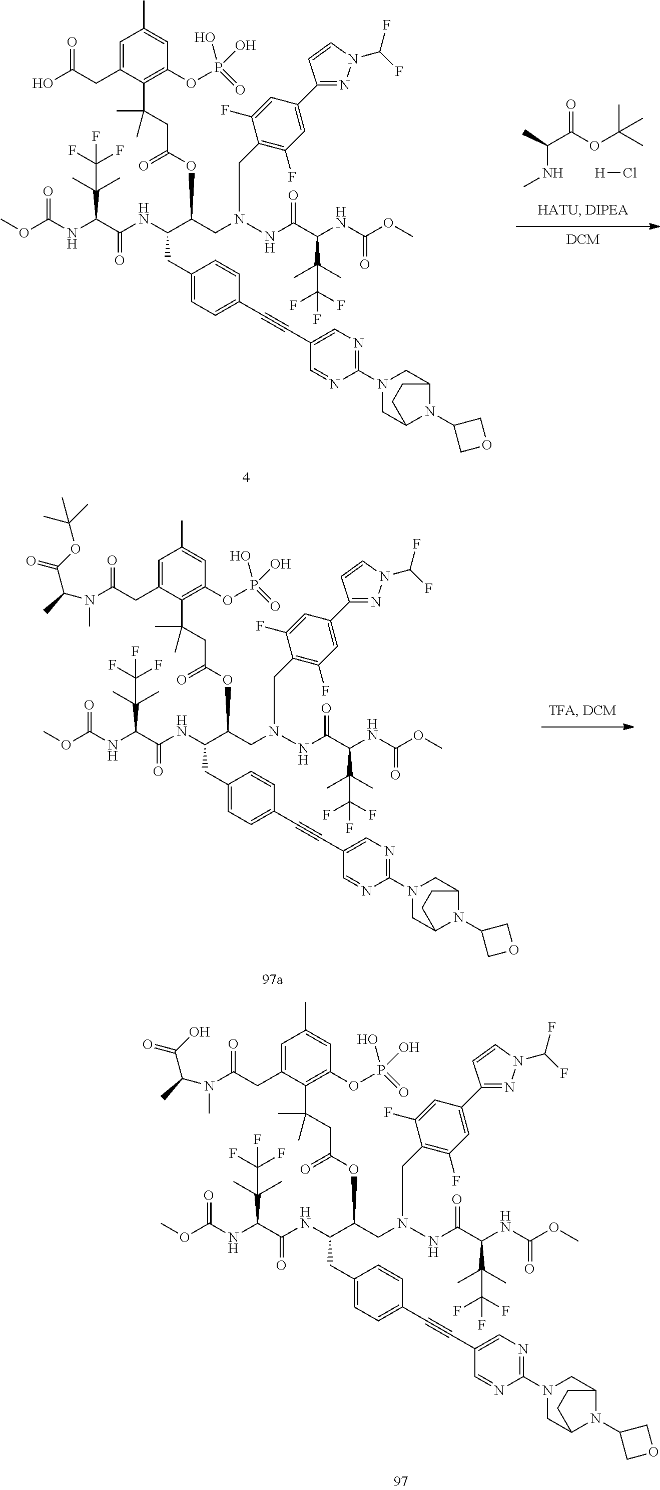

### Example 97

N-(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-N-methyl-L-alanine 97 was synthesized using the method described in Example 66, substituting tert-butyl methyl-L-alaninate hydrochloride for di-tert-butyl L-glutamate hydrochloride. MS (m/z): 785.20 [M+2H]²⁺. ¹H NMR (400 MHz, Methanol-d4) δ 8.55 (s, 2H), 8.12 (d, J=2.8 Hz, 1H), 7.73-7.38 (m), 7.34-7.23 (m), 7.15-7.05 (m), 6.99-6.86 (m), 6.71-6.55 (m), 5.20-5.10 (m), 5.02-4.93 (m), 4.81-4.75 (m), 4.60-4.46 (m), 4.40-4.24 (m), 4.24-4.10 (m), 4.04-3.90 (m), 3.81-3.64 (m), 3.60-3.44 (m), 3.07-2.87 (m), 2.87-2.70 (m), 2.36-2.17 (m), 2.13-1.95 (m), 1.79-1.69 (m), 1.62 (s, 3H), 1.51-1.39 (m), 1.25-1.11 (m), 1.06-0.95 (m). ¹⁹F NMR (376 MHz, Methanol-d4) δ −77.18 (s), −77.77 (s), −96.87 (d, J=5.8 Hz), −97.03 (d, J=5.9 Hz), −114.47 (s).

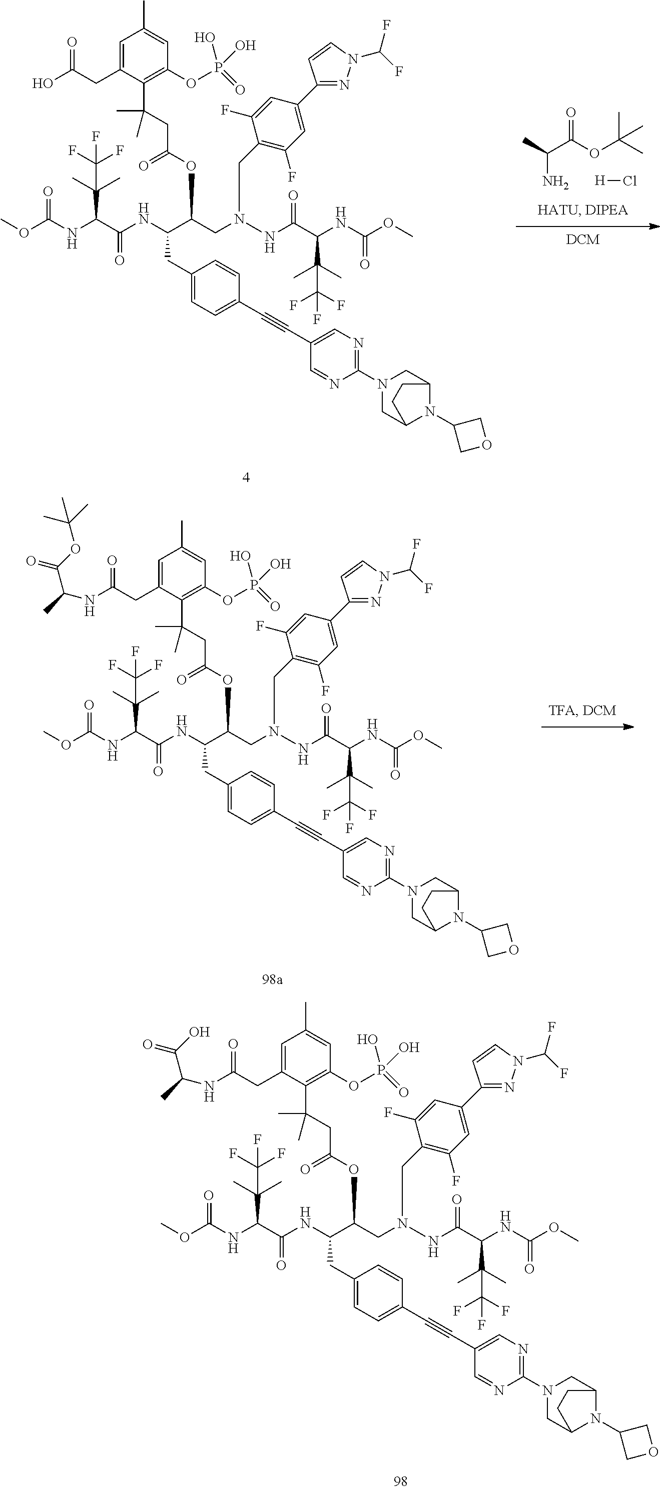

### Example 98

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-alanine 98 was synthesized using the method described in Example 66, substituting tert-butyl L-alaninate hydrochloride for di-tert-butyl L-glutamate hydrochloride. MS (m/z): 778.20 [M+2H]²⁺. ¹H NMR (400 MHz, Methanol-d4) δ 8.54 (s, 2H), 8.12 (d, J=2.8 Hz, 1H), 7.72-7.39 (m), 7.32-7.27 (m), 7.35-7.24 (m, 3H), 7.15-7.08 (m), 6.99-6.89 (m), 6.78-6.72 (m), 5.03-4.94 (m), 4.83-4.73 (m), 4.61-4.44 (m), 4.42-4.33 (m), 4.33-4.27 (m), 4.26-4.10 (m), 4.00-3.89 (m), 3.83-3.71 (m), 3.71-3.64 (m), 3.59-3.48 (m), 3.48-3.38 (m), 3.04-2.92 (m), 2.90-2.81 (m), 2.81-2.70 (m), 2.39-2.28 (m), 2.28-2.19 (m), 2.16-2.05 (m), 2.05-1.95 (m), 1.78 (s, 3H), 1.65 (s, 3H), 1.45 v 1.37 (m), 1.24-1.14 (m), 1.05-0.95 (m). ¹⁹F NMR (376 MHz, Methanol-d4) δ −77.19 (s), −77.97 (s), −96.87 (d, J=6.8 Hz), −97.03 (d, J=6.8 Hz), −114.46 (s).

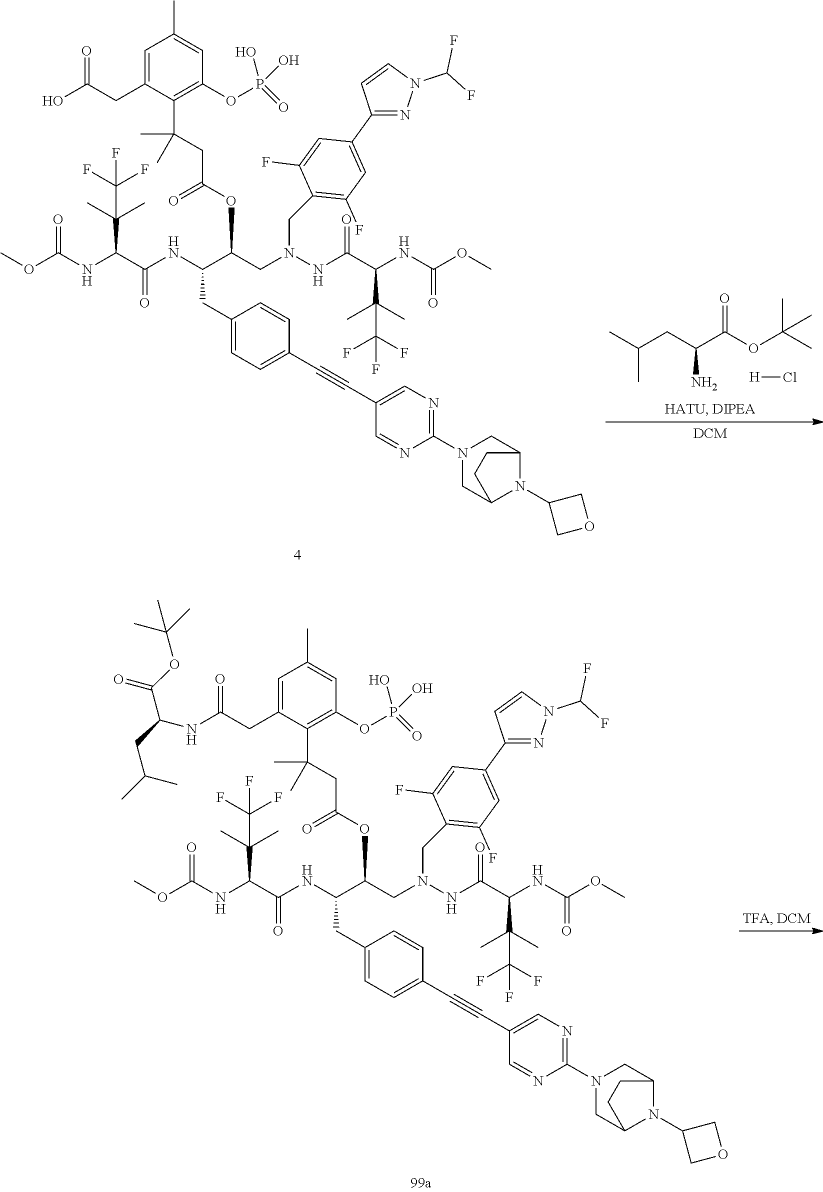

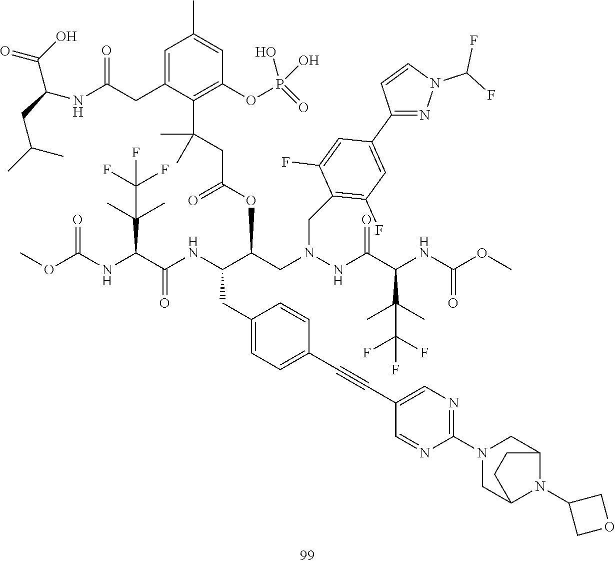 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CC(C)C)C(=O)O)cc(C)cc1OP(=O)(O)O)C(C)(C)C(F)(F)F]

### Example 99

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-L-leucine 99 was synthesized using the method described in Example 66, substituting tert-butyl L-leucinate hydrochloride for di-tert-butyl L-glutamate hydrochloride. MS (m/z): 799.20 [M+2H]²⁺. ¹H NMR (400 MHz, Methanol-d4) δ 8.55 (s, 2H), 8.12 (d, J=2.7 Hz, 1H), 7.72-7.49 (m), 7.35-7.24 (m), 7.15-7.07 (m), 6.99-6.89 (m), 6.77-6.68 (m), 5.02-4.94 (m), 4.84-4.75 (m), 4.59-4.50 (m), 4.50-4.45 (m), 4.39-4.33 (m), 4.33-4.25 (m), 4.25-4.21 (m), 4.21-4.12 (m), 3.99-3.91 (m), 3.80-3.71 (m), 3.71-3.65 (m), 3.62-3.41 (m), 3.03-2.92 (m), 2.87-2.73 (m), 2.35-2.18 (m), 2.15-1.97 (m), 1.97-1.88 (m), 1.79 (s, 3H), 1.65 (s, 3H), 1.56-1.44 (m), 1.27-1.14 (m), 1.04-0.88 (m). ¹⁹F NMR (376 MHz, Methanol-d4) δ −77.17 (s), −77.80 (s), −96.87 (d, J=7.5 Hz), −97.03 (d, J=7.8 Hz), −114.51 (s).

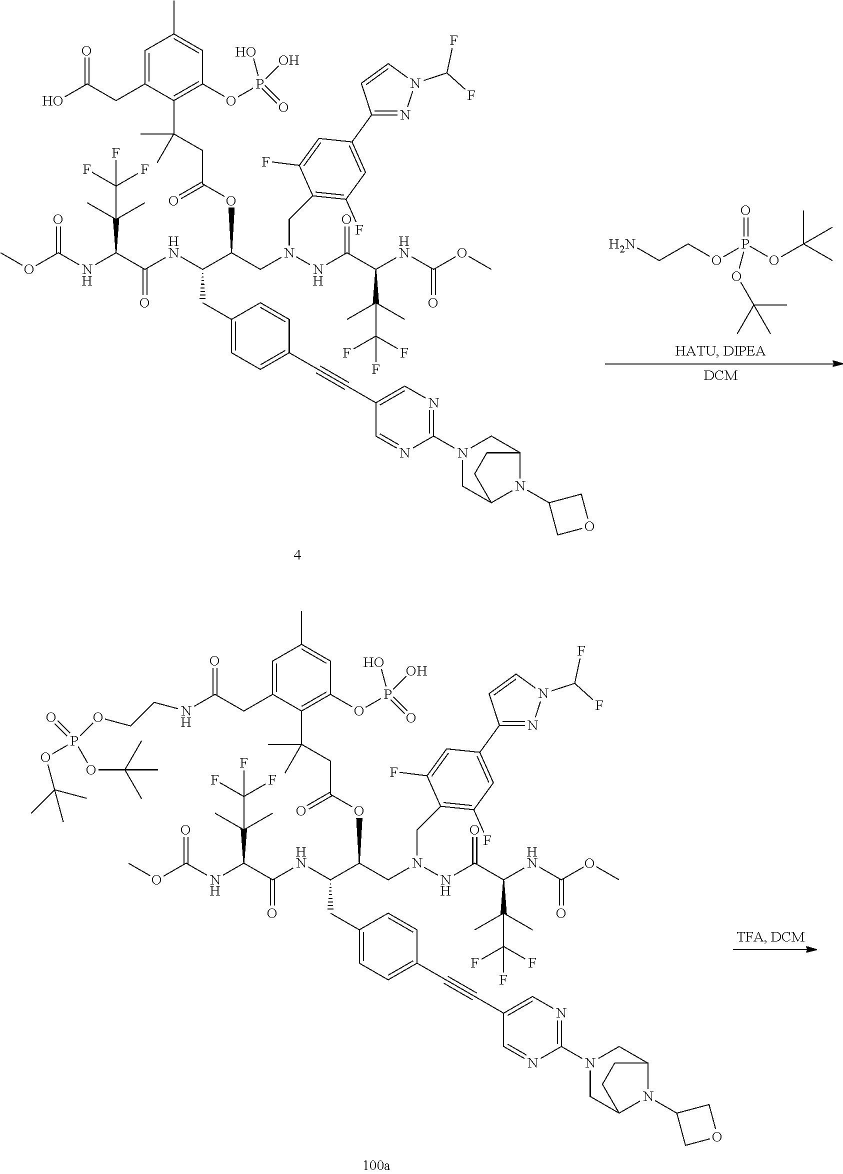 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OP(=O)(OCCN)OC(C)(C)C ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCOP(=O)(OC(C)(C)C)OC(C)(C)C)cc(C)cc1OP(=O)(O)O)C(C)(C)C(F)(F)F]

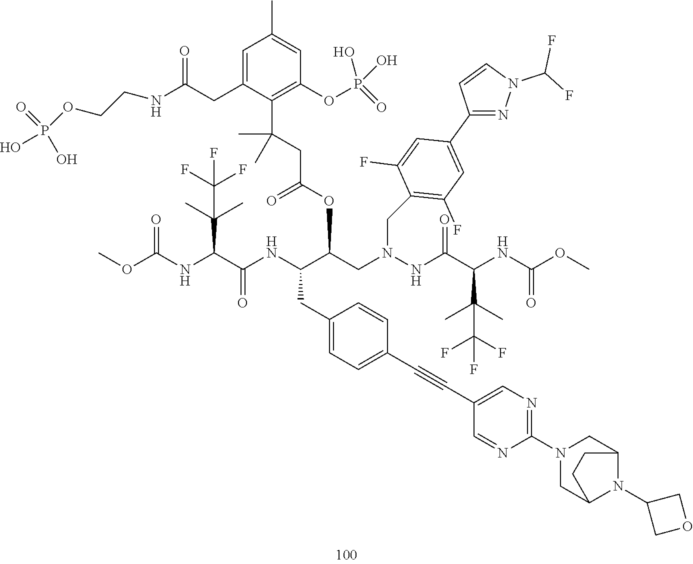 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCCOP(=O)(O)O)cc(C)cc1OP(=O)(O)O)C(C)(C)C(F)(F)F]

### Example 100

(5S,10S,11S,14S)-8-(4-(1-(difluoromethyl)-1H-pyrazol-3-yl)-2,6-difluorobenzyl)-11-(4-((2-(8-(oxetan-3-yl)-3,8-diazabicyclo[3.2.1]octan-3-yl)pyrimidin-5-yl)ethynyl)benzyl)-3,6,13,16-tetraoxo-5,14-bis(1,1,1-trifluoro-2-methylpropan-2-yl)-2,17-dioxa-4,7,8,12,15-pentaazaoctadecan-10-yl 3-methyl-3-(4-methyl-2-(2-oxo-2-((2-(phosphonooxy)ethyl)amino)ethyl)-6-(phosphonooxy)phenyl)butanoate 100 was synthesized using the method described in Example 66, substituting 2-aminoethyl di-tert-butyl phosphate for di-tert-butyl L-glutamate hydrochloride. MS (m/z): 804.20 [M+2H]²⁺. ¹H NMR (400 MHz, Methanol-d4) δ 8.55 (s, 2H), 8.12 (d, J=2.7 Hz, 1H), 7.73-7.38 (m), 7.36-7.25 (m), 7.16-7.07 (m), 6.99-6.87 (m), 6.73-6.67 (m), 5.03-4.94 (m), 4.83-4.75 (m), 4.61-4.46 (m), 4.43-4.34 (m), 4.34-4.26 (m), 4.24-4.11 (m), 4.08-3.99 (m), 3.98-3.90 (m), 3.82-3.64 (m), 3.60-3.40 (m), 3.04-2.91 (m), 2.91-2.81 (m), 2.81-2.70 (m), 2.38-2.280 (m), 2.28-2.19 (m), 2.19-2.05 (m), 2.05-1.95 (m), 1.77 (s, 3H), 1.65 (s, 3H), 1.25-1.13 (m), 1.03-0.93 (m). ¹⁹F NMR (376 MHz, Methanol-d4) δ −77.17 (s), −77.85 (s), −96.87 (d, J=8.3 Hz), −97.03 (d, J=8.3 Hz), −114.47 (s).

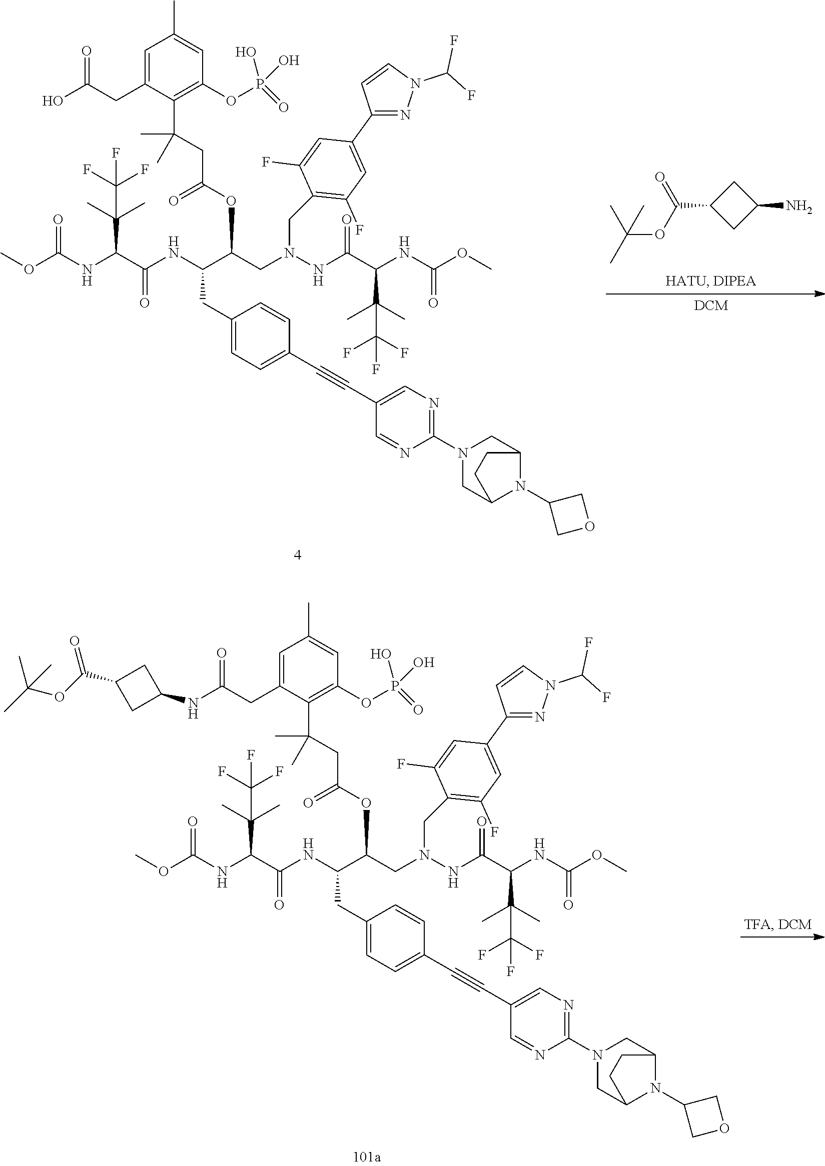 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OC(=O)[C@H]1C[C@H](N)C1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H]2C[C@H](C(=O)OC(C)(C)C)C2)cc(C)cc1OP(=O)(O)O)C(C)(C)C(F)(F)F]

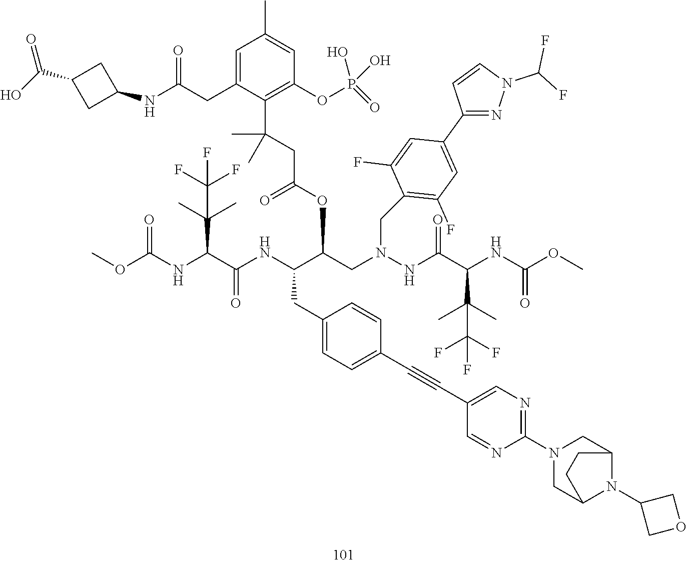 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H]2C[C@H](C(=O)O)C2)cc(C)cc1OP(=O)(O)O)C(C)(C)C(F)(F)F]

### Example 101

(1S,3R)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)cyclobutane-1-carboxylic acid 101 was synthesized using the method described in Example 66, substituting tert-butyl trans-3-aminocyclobutane-1-carboxylate for di-tert-butyl L-glutamate hydrochloride. MS (m/z): 791.20 [M+2H]²⁺. ¹H NMR (400 MHz, Methanol-d4) δ 8.55 (s, 2H), 8.12 (d, J=2.7 Hz, 1H), 7.72-7.39 (m), 7.36-7.23 (m), 7.16-7.08 (m), 6.98-6.88 (m), 6.72-6.67 (m), 5.02-4.93 (m), 4.81-4.75 (m), 4.61-4.48 (m), 4.40-4.33 (m), 4.32-4.25 (m), 4.25-4.14 (m), 4.14-4.05 (m), 3.98-3.90 (m), 3.81-3.71 (m), 3.68 (s, 3H), 3.59-3.41 (m), 3.06-2.96 (m), 2.96-2.85 (m), 2.85-2.75 (m), 2.62-2.52 (m), 2.35-2.20 (m), 2.20-2.06 (m), 2.06-1.95 (m), 1.76 (s, 3H), 1.64 (s, 3H), 1.24-1.17 (m), 0.99 (s, 3H). ¹⁹F NMR (376 MHz, Methanol-d4) δ −77.19 (s), −77.80 (s), −96.87 (d, J=7.7 Hz), −97.03 (d, J=7.7 Hz), −114.49 (s).

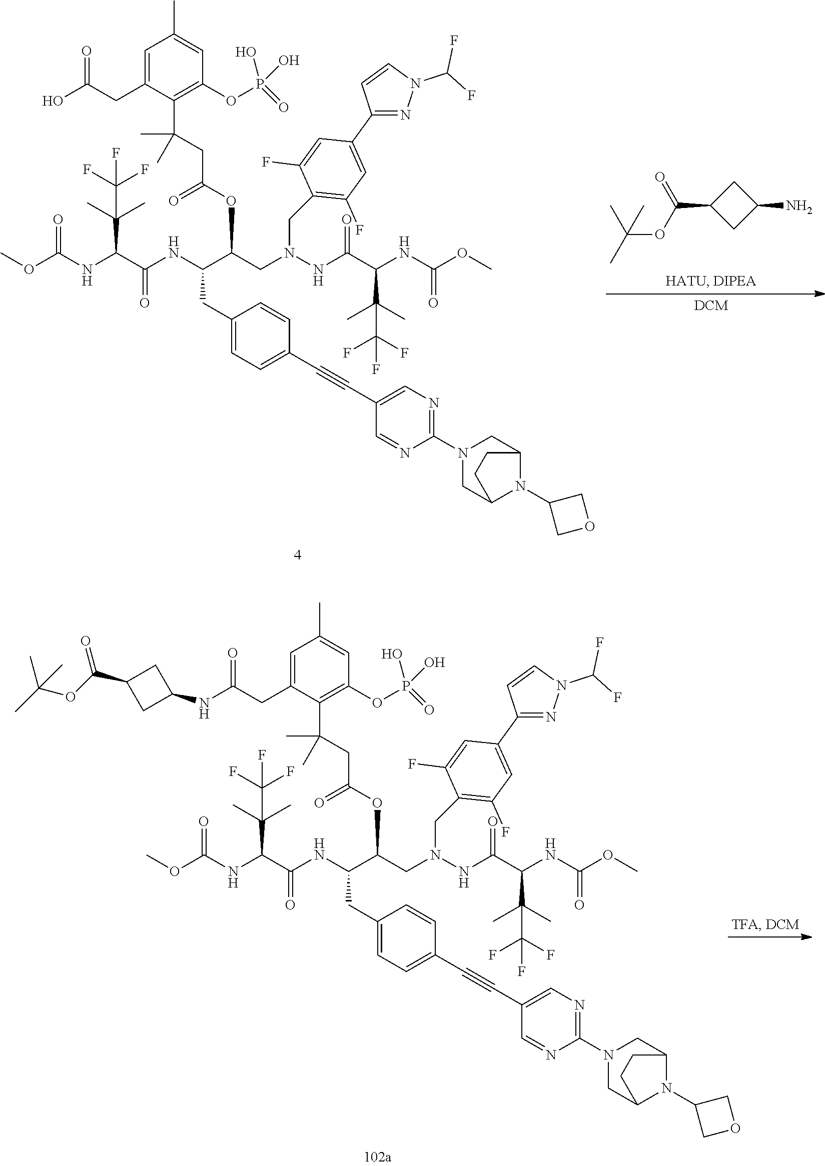 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OC(=O)[C@H]1C[C@@H](N)C1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H]2C[C@@H](C(=O)OC(C)(C)C)C2)cc(C)cc1OP(=O)(O)O)C(C)(C)C(F)(F)F]

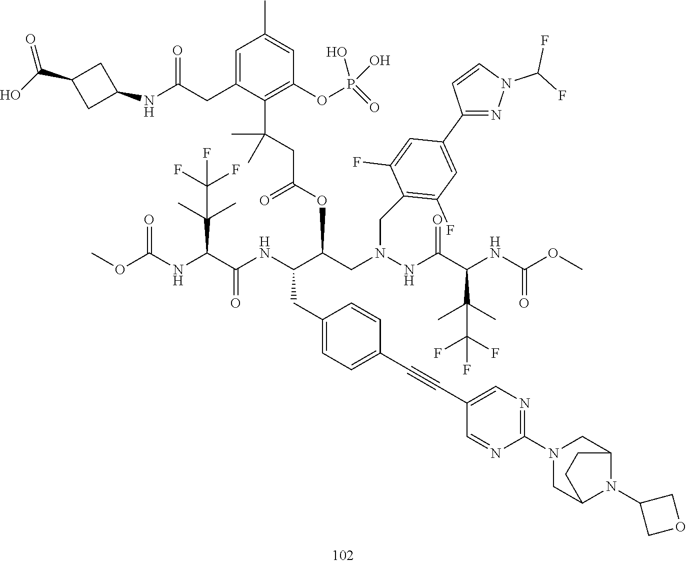 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@H]2C[C@@H](C(=O)O)C2)cc(C)cc1OP(=O)(O)O)C(C)(C)C(F)(F)F]

### Example 102

(1R,3R)-3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)cyclobutane-1-carboxylic acid 102 was synthesized using the method described in Example 66, substituting tert-butyl cis 3-aminocyclobutane-1-carboxylate for di-tert-butyl L-glutamate hydrochloride. MS (m/z): 791.20 [M+2H]²⁺. ¹H NMR (400 MHz, Methanol-d4) δ 8.55 (s, 2H), 8.12 (d, J=2.7 Hz, 1H), 7.72-7.39 (m), 7.35-7.23 (m), 7.15-7.09 (m), 6.96 (d, J=2.8 Hz, 1H), 6.91 (d, J=9.9 Hz, 1H), 6.70-6.65 (m), 5.02-4.93 (m), 4.82-4.75 (m), 4.60-4.48 (m), 4.40-4.27 (m), 4.24-4.14 (m), 4.10-4.01 (m), 3.97-3.89 (m), 3.81-3.70 (m), 3.68 (s, 3H), 3.60-3.47 (m), 3.47-3.37 (m), 3.06-2.95 (m), 2.95-2.73 (m), 2.62-2.51 (m), 2.40-2.30 (m), 2.30-2.11 (m), 2.10-1.93 (m), 1.76 (s, 3H), 1.64 (s, 3H), 1.25-1.15 (m), 1.00 (s, 3H). ¹⁹F NMR (376 MHz, Methanol-d4) δ −77.18 (s), −77.800 (s), −96.87 (d, J=8.3 Hz), −97.03 (d, J=8.0 Hz), −114.49 (s).

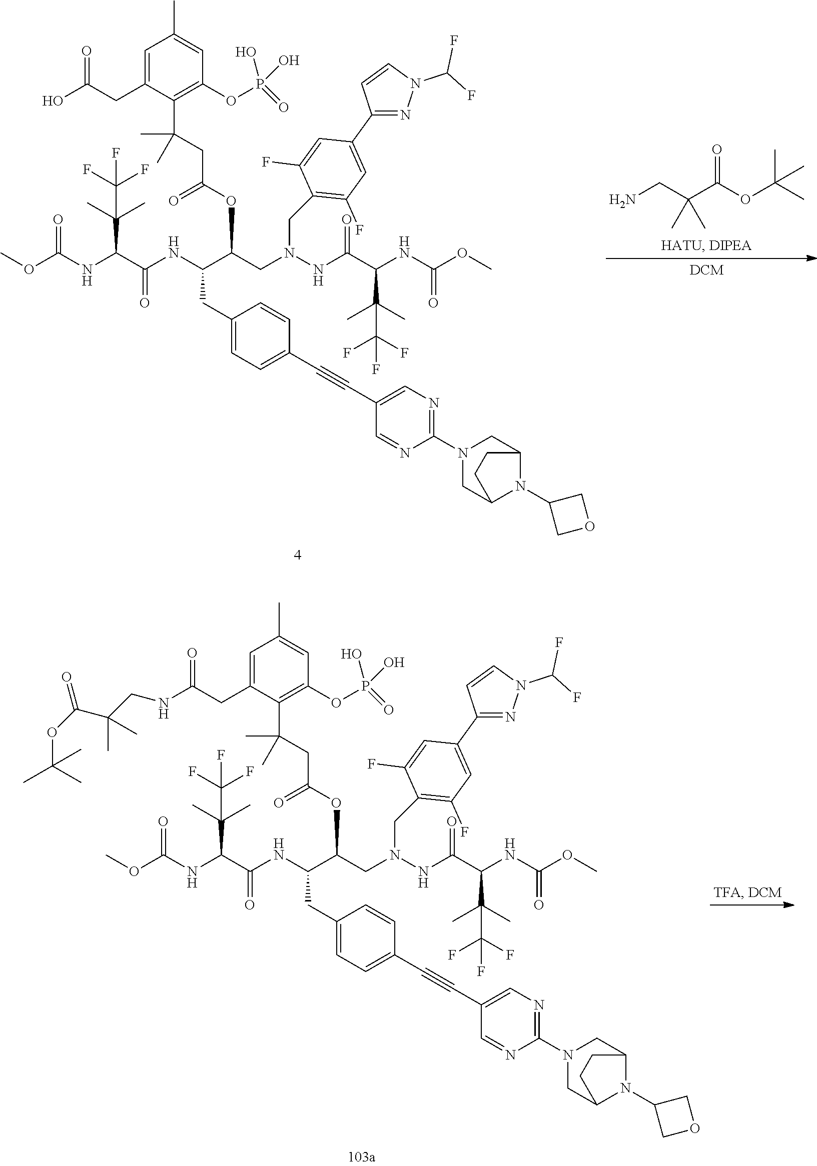 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OC(=O)C(C)(C)CN ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(C)(C)C(=O)OC(C)(C)C)cc(C)cc1OP(=O)(O)O)C(C)(C)C(F)(F)F]

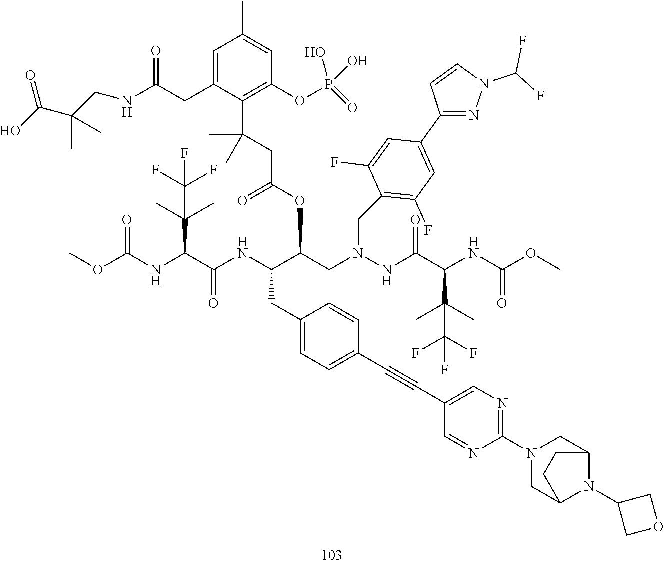 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(C)(C)C(=O)O)cc(C)cc1OP(=O)(O)O)C(C)(C)C(F)(F)F]

### Example 103

3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-2,2-dimethylpropanoic acid 103 was synthesized using the method described in Example 66, substituting tert-butyl 3-amino-2,2-dimethylpropanoate for di-tert-butyl L-glutamate hydrochloride. MS (m/z): 792.20 [M+2H]²⁺. ¹H NMR (400 MHz, Methanol-d4) δ 8.55 (s, 2H), 8.12 (d, J=2.8 Hz, 1H), 7.72-7.39 (m), 7.35-7.26 (m), 7.15-7.08 (m), 6.99-6.90 (m), 6.73-6.68 (m), 5.02-4.93 (m), 4.82-4.74 (m), 4.60-4.49 (m), 4.40-4.27 (m), 4.24-4.12 (m), 3.99-3.91 (m), 3.80-3.64 (m), 3.57-3.42 (m), 3.40-3.35 (m), 3.03-2.94 (m), 2.88-2.74 (m), 2.37-2.28 (m), 2.28-2.19 (m), 2.15-1.95 (m), 1.78 (s, 3H), 1.64 (s, 3H), 1.24-1.13 (m), 1.01 (s, 3H). ¹⁹F NMR (376 MHz, Methanol-d4) δ −77.18 (s), −77.77 (s), −96.87 (d, J=6.5 Hz), −97.03 (d, J=6.6 Hz), −114.45 (s).

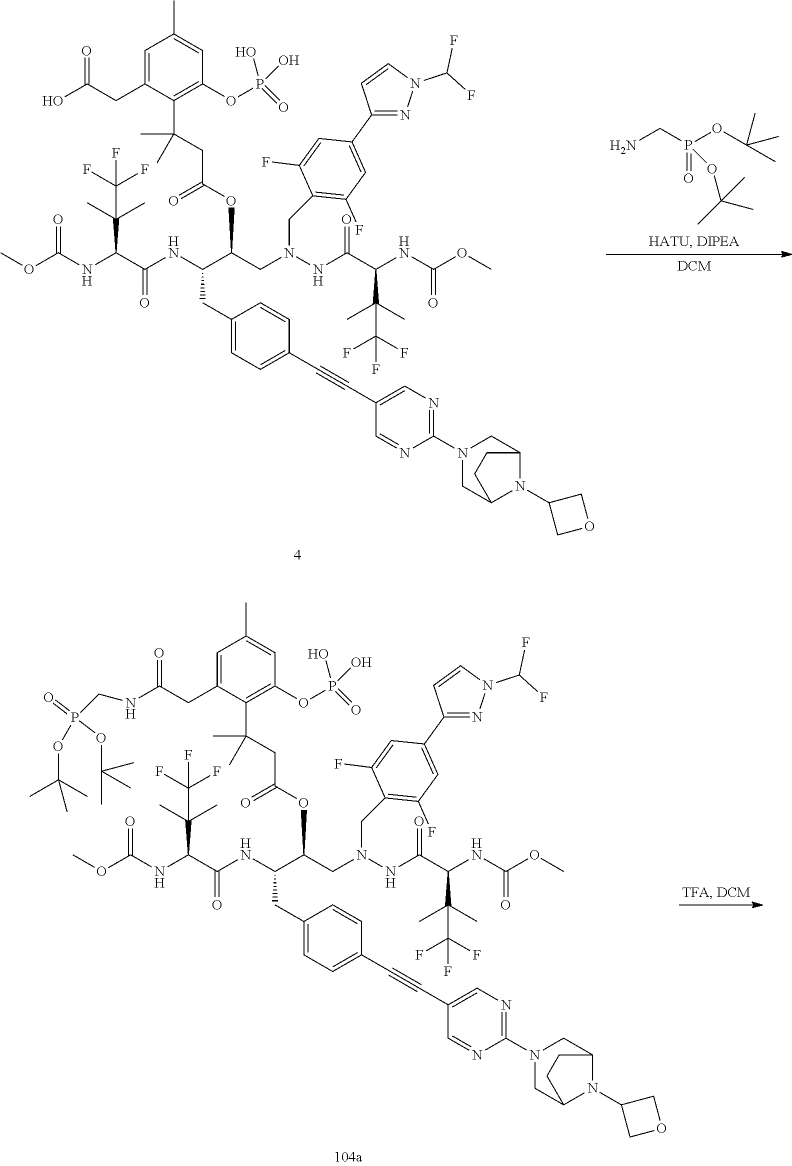 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OP(=O)(CN)OC(C)(C)C ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCP(=O)(OC(C)(C)C)OC(C)(C)C)cc(C)cc1OP(=O)(O)O)C(C)(C)C(F)(F)F]

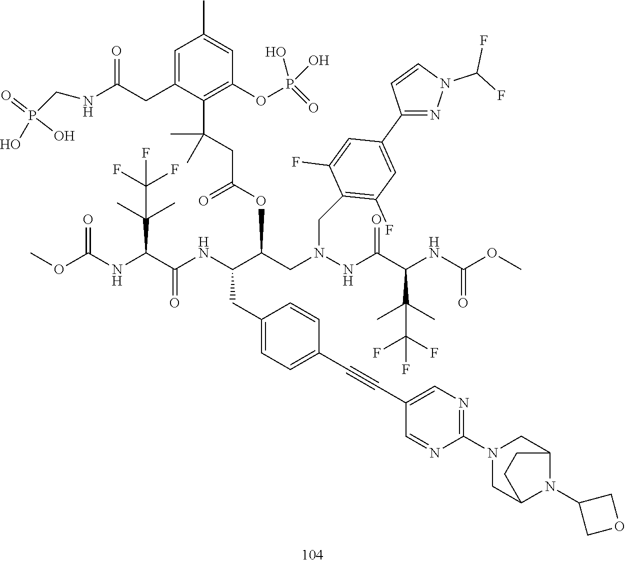 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCP(=O)(O)O)cc(C)cc1OP(=O)(O)O)C(C)(C)C(F)(F)F]

### Example 104

((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)methyl)phosphonic acid 104 was synthesized using the method described in Example 66, substituting di-tert-butyl (aminomethyl)phosphonate for di-tert-butyl L-glutamate hydrochloride. MS (m/z): 789.20 [M+2H]²⁺. ¹H NMR (400 MHz, Methanol-d4) δ 8.55 (s, 2H), 8.12 (d, J=2.8 Hz, 1H), 7.72-7.39 (m), 7.35-7.26 (m), 7.15-7.08 (m), 6.99-6.90 (m), 6.77-6.67 (m), 5.02-4.93 (m), 4.82-4.74 (m), 4.60-4.49 (m), 4.40-4.27 (m), 4.24-4.12 (m), 3.99-3.91 (m), 3.80-3.64 (m), 3.64-3.46 (m), 3.03-2.94 (m), 2.85-2.74 (m), 2.37-2.28 (m), 2.28-2.19 (m), 2.15-1.95 (m), 1.78 (s, 3H), 1.64 (s, 3H), 1.24-1.13 (m), 1.04-0.96 (m). ¹⁹F NMR (376 MHz, Methanol-d4) δ −77.16 (s), −77.81 (s), −96.87 (d, J=6.5 Hz), −97.03 (d, J=6.6 Hz), −114.46 (s).

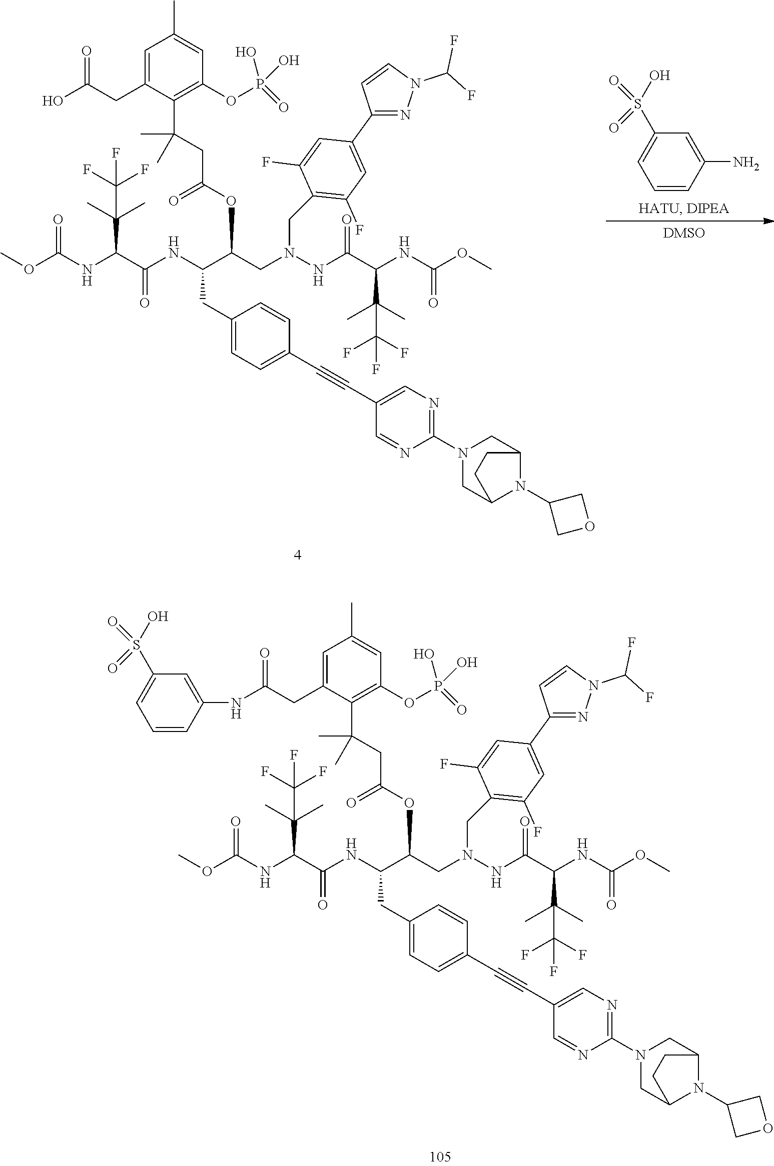 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; Nc1cccc(S(=O)(=O)O)c1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cccc(S(=O)(=O)O)c2)cc(C)cc1OP(=O)(O)O)C(C)(C)C(F)(F)F]

### Example 105

To a suspension of 4 (0.032 mmol) in DMSO (0.75 mL) was added a solution of 3-aminobenzenesulfonic acid (0.097 mmol), N,N-diisopropylethylamine (0.21 mmol) in DMSO (0.75 mL) followed by HATU (0.128 mmol). The reaction was stirred at 45° C. for 4 hours and then at room temperature for 12 hours. The residue obtained was diluted with DMSO and purified by reverse-phase HPLC (eluent: water/MeCN 0.1% TFA) to afford 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)benzenesulfonic acid (105). MS (m/z): 820.20 [M+2H]²⁺. ¹H NMR (400 MHz, Methanol-d4) δ 8.55-8.48 (m), 8.11 (d, J=2.8 Hz, 1H), 7.97-7.87 (m), 7.72-7.52 (m), 7.48-7.41 (m), 7.41-7.33 (m), 7.33-7.24 (m), 7.13-7.01 (m), 6.95 (d, J=2.8 Hz, 1H), 6.80-6.75 (m), 5.03-4.92 (m), 4.86-4.71 (m), 4.53-4.43 (m), 4.42-4.34 (m), 4.34-4.26 (m), 4.25-4.11 (m), 4.03-3.91 (m), 3.81-3.70 (m), 3.67 (s, 3H), 3.52-3.41 (m), 3.05-2.96 (m), 2.93-2.79 (m), 2.40-2.30 (m), 2.29-2.18 (m), 2.18-2.06 (m), 2.03-1.94 (m), 1.83 (s, 3H), 1.67 (s, 3H), 1.25-1.11 (m), 1.00 (s, 3H). ¹⁹F NMR (376 MHz, Methanol-d4) δ−77.20 (s), −77.72 (s), −96.86 (d, J=8.9 Hz), −97.02 (d, J=9.0 Hz), −114.53 (s).

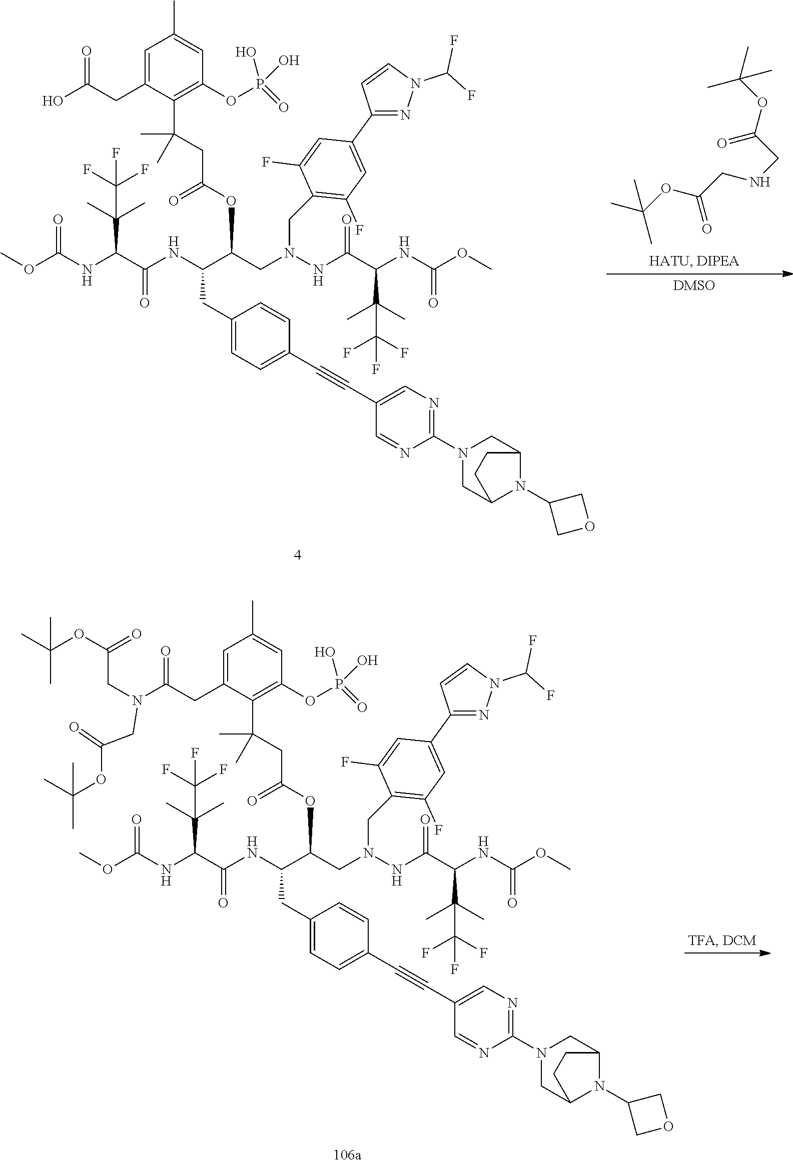 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OC(=O)CNCC(=O)OC(C)(C)C ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(CC(=O)OC(C)(C)C)CC(=O)OC(C)(C)C)cc(C)cc1OP(=O)(O)O)C(C)(C)C(F)(F)F]

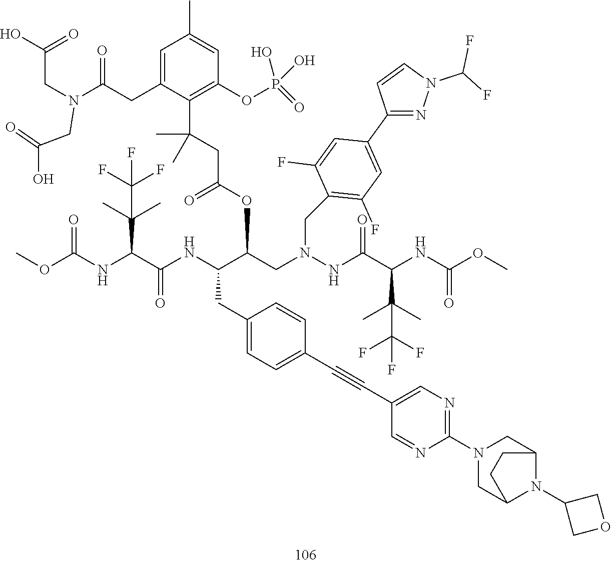 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N(CC(=O)O)CC(=O)O)cc(C)cc1OP(=O)(O)O)C(C)(C)C(F)(F)F]

### Example 106

2,2′-((2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)azanediyl)diacetic acid 106 was synthesized using the method described in Example 66, substituting di-tert-butyl 2,2′-azanediyldiacetate for di-tert-butyl L-glutamate hydrochloride. MS (m/z): 800.20 [M+2H]²⁺. ¹H NMR (400 MHz, Methanol-d4) δ 8.54 (s, 2H), 8.12 (d, J=2.8 Hz, 1H), 7.72-7.39 (m), 7.34-7.22 (m), 7.14-7.07 (m), 6.99-6.88 (m), 6.72-6.65 (m), 5.02-4.95 (m), 4.81-4.71 (m), 4.59-4.48 (m), 4.39-4.25 (m), 4.25-4.13 (m), 4.00-3.88 (m), 3.81-3.71 (m), 3.68 (s, 3H), 3.57-3.40 (m), 3.00-2.90 (m), 2.84-2.74 (m), 2.38-2.29 (m), 2.28-2.19 (m), 2.13-1.95 (m), 1.75 (s, 3H), 1.62 (s, 3H), 1.24-1.11 (m), 1.04 (s, 3H). ¹⁹F NMR (376 MHz, Methanol-d4) δ−77.20 (s), −78.02 (s), −96.87 (d, J=5.2 Hz), −97.03 (d, J=5.2 Hz), −114.40 (s).

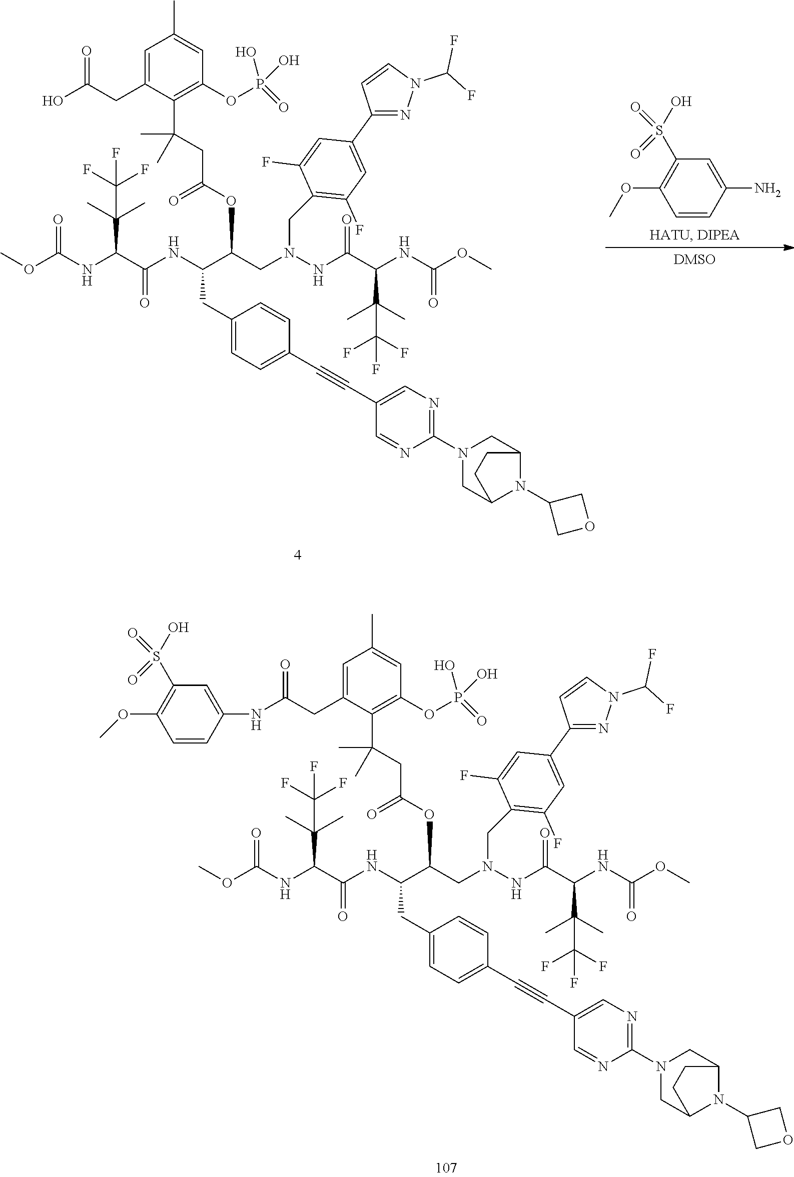 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; COc1ccc(N)cc1S(=O)(=O)O ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2ccc(OC)c(S(=O)(=O)O)c2)cc(C)cc1OP(=O)(O)O)C(C)(C)C(F)(F)F]

### Example 107

To a suspension of 4 (0.063 mmol) in DMSO (1 mL) was added a solution of 5-amino-2-methoxybenzenesulfonic acid (0.131 mmol), N,N-diisopropylethylamine (0.375 mmol) in DMSO (1 mL) followed by HATU (0.067 mmol). The reaction was stirred at 45° C. for 4 hours and then purified by reverse-phase HPLC (eluent: water/MeCN 0.1% TFA) to afford 5-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-2-methoxybenzenesulfonic acid (107). MS (m/z): 835.20 [M+2H]²⁺. ¹H NMR (400 MHz, Methanol-d4) δ 8.54 (s, 2H), 8.15-8.05 (m), 7.72-7.42 (m), 7.42-7.37 (m), 7.34-7.24 (m), 7.12-7.04 (m), 7.00-6.91 (m), 6.90-6.82 (m), 5.02-4.93 (m), 4.86-4.72 (m), 4.53-4.42 (m), 4.42-4.27 (m), 4.25-4.10 (m), 3.98-3.83 (m), 3.81-3.66 (m), 3.53-3.44 (m), 3.01-2.92 (m), 2.84-2.73 (m), 2.36-2.19 (m), 2.17-2.12 (m), 2.05-1.94 (m), 1.85 (s, 3H), 1.66 (s, 3H), 1.25-1.12 (m), 1.02 (s, 3H). ¹⁹F NMR (376 MHz, Methanol-d4) δ −77.17 (s), −77.79 (s), −96.86 (d, J=6.1 Hz), −97.02 (d, J=5.7 Hz), −114.36 (s).

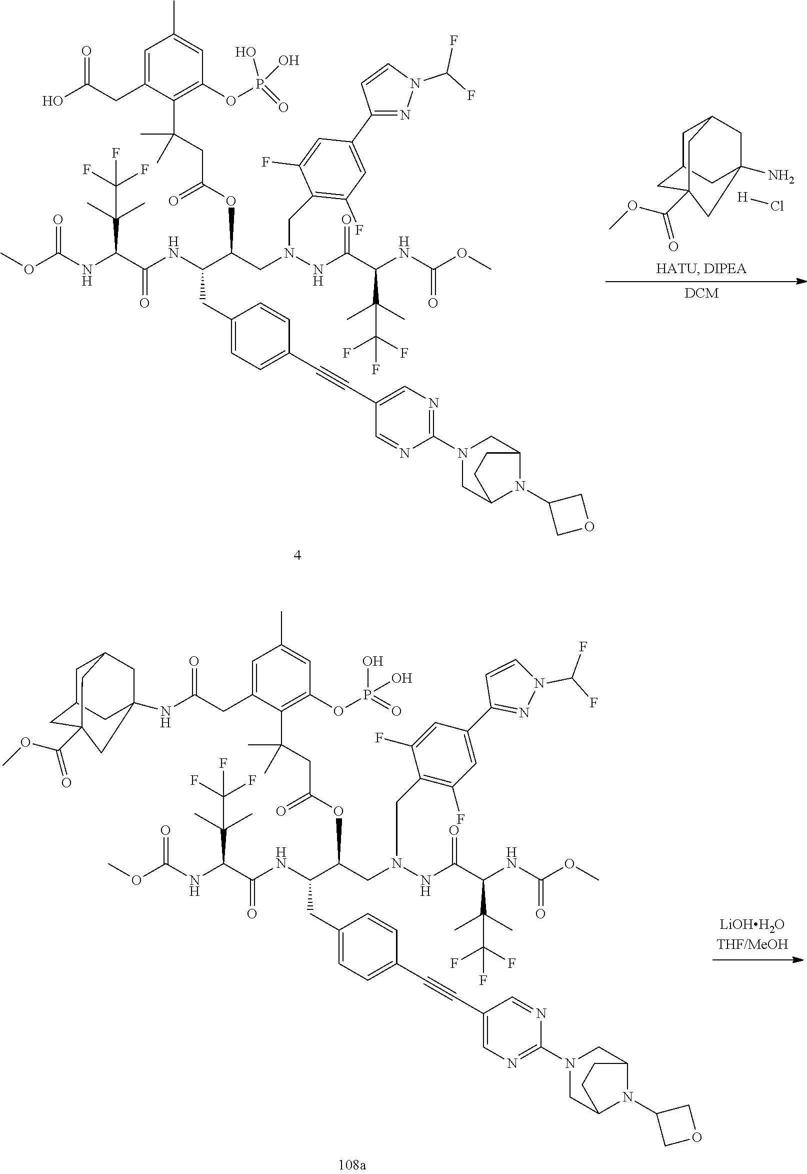

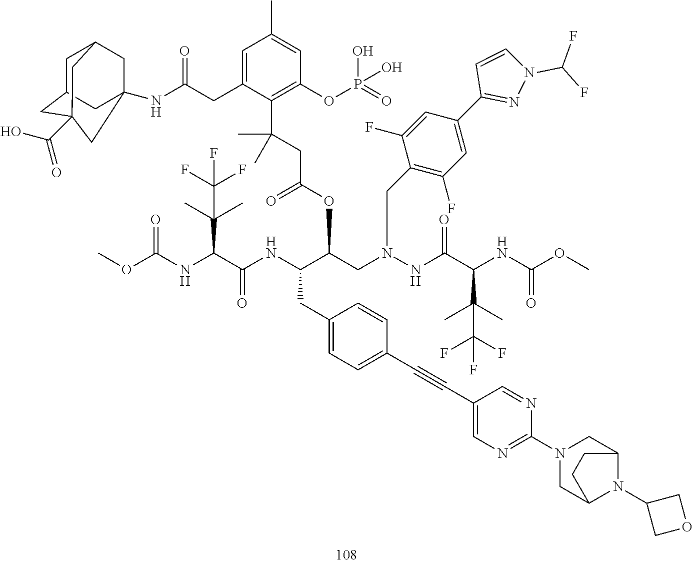 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NC23CC4CC(C2)CC(C(=O)O)(C4)C3)cc(C)cc1OP(=O)(O)O)C(C)(C)C(F)(F)F]

### Example 108

3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)adamantane-1-carboxylic acid 108 was synthesized using the method described in Example 67, substituting methyl 3-aminoadamantane-1-carboxylate hydrochloride for dimethyl piperidine-3,5-dicarboxylate. MS (m/z): 831.30 [M+2H]²⁺. ¹H NMR (400 MHz, Methanol-d4) δ 8.55 (s, 2H), 8.12 (d, J=2.8 Hz, 1H), 7.72-7.39 (m), 7.34-7.24 (m), 7.15-7.08 (m), 6.99-6.87 (m), 6.73-6.67 (m), 5.52-5.50 (m), 5.02-4.95 (m), 4.84-4.74 (m), 4.58-4.48 (m), 4.40-4.33 (m), 4.32-4.26 (m), 4.24-4.14 (m), 4.07-3.90 (m), 3.80-3.64 (m), 3.56-3.42 (m), 3.05-2.94 (m), 2.92-2.76 (m), 2.40-2.30 (m), 2.28-2.20 (m), 2.20-2.07 (m), 2.05-1.97 (m), 1.87-1.76 (m), 1.70-1.62 (m), 1.25-1.13 (m), 1.03-0.95 (m). ¹⁹F NMR (376 MHz, Methanol-d4) δ−77.16 (s), −77.79 (s), −96.86 (d, J=9.3 Hz), −97.02 (d, J=9.2 Hz), −114.45 (s).

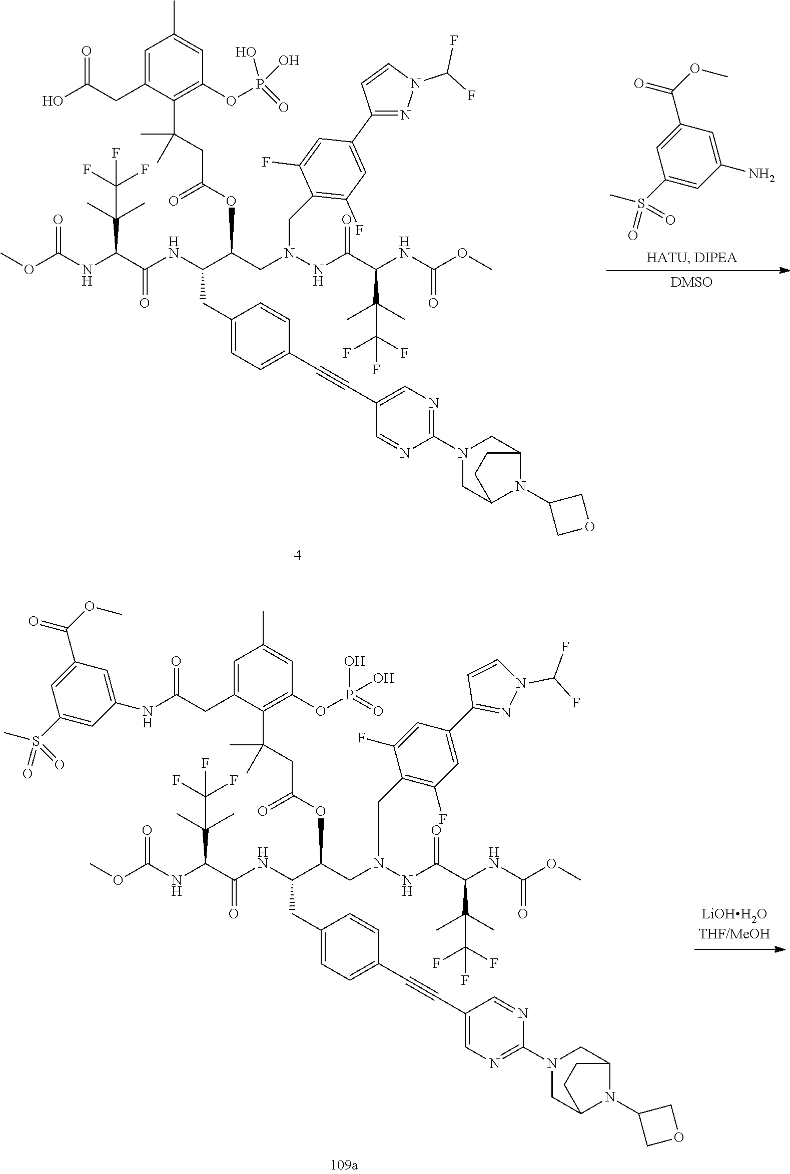 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; COC(=O)c1cc(N)cc(S(C)(=O)=O)c1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)OC)cc(S(C)(=O)=O)c2)cc(C)cc1OP(=O)(O)O)C(C)(C)C(F)(F)F]

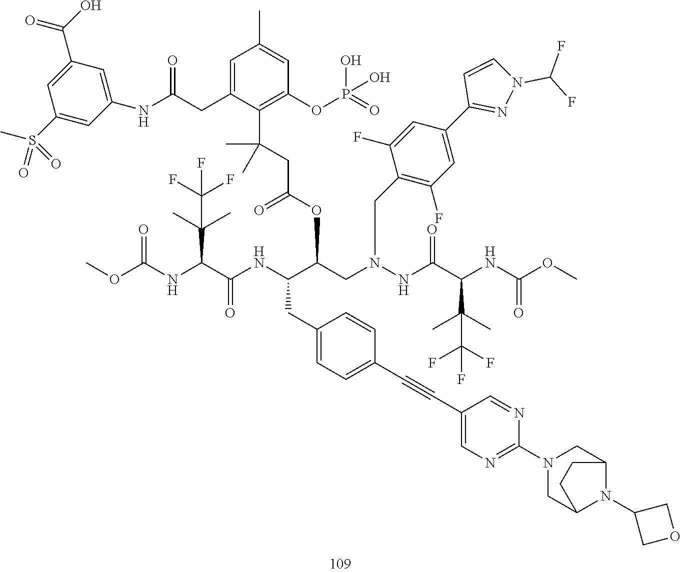 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)Nc2cc(C(=O)O)cc(S(C)(=O)=O)c2)cc(C)cc1OP(=O)(O)O)C(C)(C)C(F)(F)F]

### Example 109

Synthesis of methyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-5-(methylsulfonyl)benzoate (109a): To a suspension of 4 (0.063 mmol) in DMSO (1 mL) was added a solution of methyl 3-amino-5-(methylsulfonyl)benzoate (0.131 mmol), N,N-diisopropylethylamine (0.375 mmol) in DMSO (1 mL) followed by HATU (0.067 mmol). The reaction was stirred at 45° C. for 4 hours and then purified by reverse-phase HPLC (eluent: water/MeCN 0.1% TFA) to afford methyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-5-(methylsulfonyl)benzoate (109a). MS (m/z): 848.20 [M+2H]²⁺.

Synthesis of 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-5-(methylsulfonyl)benzoic acid (109): To a solution of methyl 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3, 8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-5-(methylsulfonyl)benzoate 109a (0.028 mmol) in a mixture of 1:1 MeOH/THF (2 mL) was added 0.5M aqueous lithium hydroxide solution (0.564 mL) and stirred at room temperature for 45 min. The reaction mixture was neutralized with dilute acetic acid and then purified by reverse-phase HPLC (eluent: water/MeCN 0.1% TFA) to afford 3-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetamido)-5-(methylsulfonyl)benzoic acid 109. MS (m/z): 841.20 [M+2H]²⁺. ¹H NMR (400 MHz, Methanol-d4) δ 8.57-8.45 (m), 8.24-8.20 (m), 8.13-8.09 (m), 7.72-7.37 (m), 7.33-7.23 (m), 7.11-7.04 (m), 6.97-6.86 (m), 6.83-6.76 (m), 5.04-4.94 (m), 4.83-4.71 (m), 4.60-4.48 (m), 4.37-4.25 (m), 4.25-4.06 (m), 3.97-3.88 (m), 3.80-3.70 (m), 3.70-3.63 (m), 3.58-3.46 (m), 3.17-3.12 (m), 3.09-2.84 (m), 2.40-2.31 (m), 2.28-2.19 (m), 2.19-2.07 (m), 2.05-1.95 (m), 1.84 (s, 3H), 1.68 (s, 3H), 1.23-1.12 (m), 1.02 (s, 3H). ¹⁹F NMR (376 MHz, Methanol-d4) δ −77.26 (s), −77.73 (s), −96.86 (d, J=13.4 Hz), −97.02 (d, J=13.5 Hz), −114.35 (s).

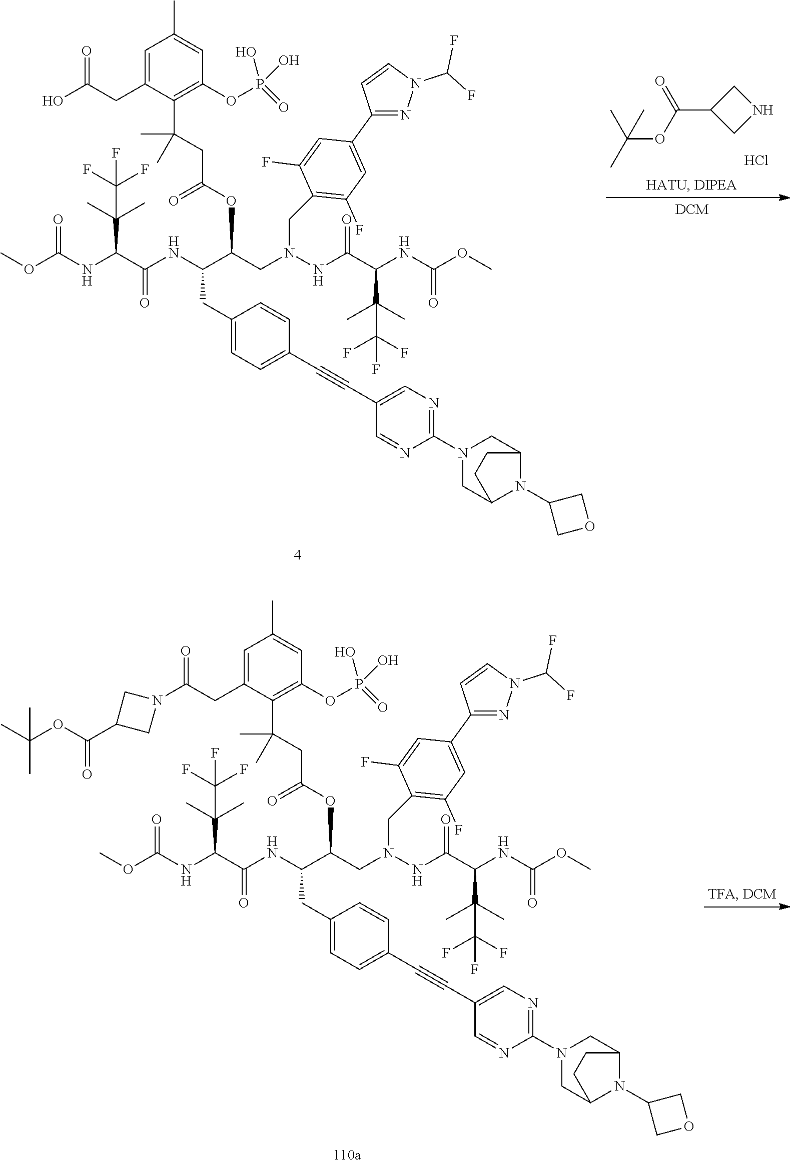 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; CC(C)(C)OC(=O)C1CNC1 ; Cl ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC(C(=O)OC(C)(C)C)C2)cc(C)cc1OP(=O)(O)O)C(C)(C)C(F)(F)F]

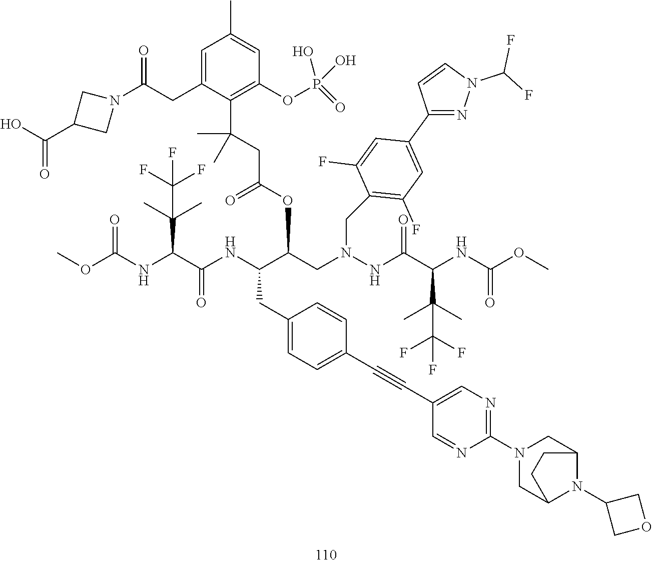 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC(C(=O)O)C2)cc(C)cc1OP(=O)(O)O)C(C)(C)C(F)(F)F]

### Example 110

1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)azetidine-3-carboxylic acid 110 was synthesized using the method described in Example 66, substituting tert-butyl azetidine-3-carboxylate hydrochloride for di-tert-butyl L-glutamate hydrochloride. MS (m/z): 784.20 [M+2H]²⁺. ¹H NMR (400 MHz, Methanol-d4) δ 8.54 (s, 2H), 8.12 (d, J=2.7 Hz, 1H), 7.72-7.39 (m), 7.34-7.22 (m), 7.15-7.07 (m), 6.99-6.89 (m), 6.68-6.63 (m), 5.02-4.95 (m), 4.86-4.74 (m), 4.58-4.50 (m), 4.43-4.33 (m), 4.33-4.27 (m), 4.27-4.11 (m), 4.11-4.00 (m), 3.98-3.90 (m), 3.80-3.65 (m), 3.65-3.44 (m), 3.02-2.85 (m), 2.80-2.70 (m), 2.34-2.19 (m), 2.15-1.95 (m), 1.75 (s, 3H), 1.64 (s, 3H), 1.25-1.11 (m), 1.05-0.95 (m). ¹⁹F NMR (376 MHz, Methanol-d4) δ −77.16 (s), −77.77 (s), −96.84-−96.92 (m), −97.00-−97.09 (m), −114.43 (s).

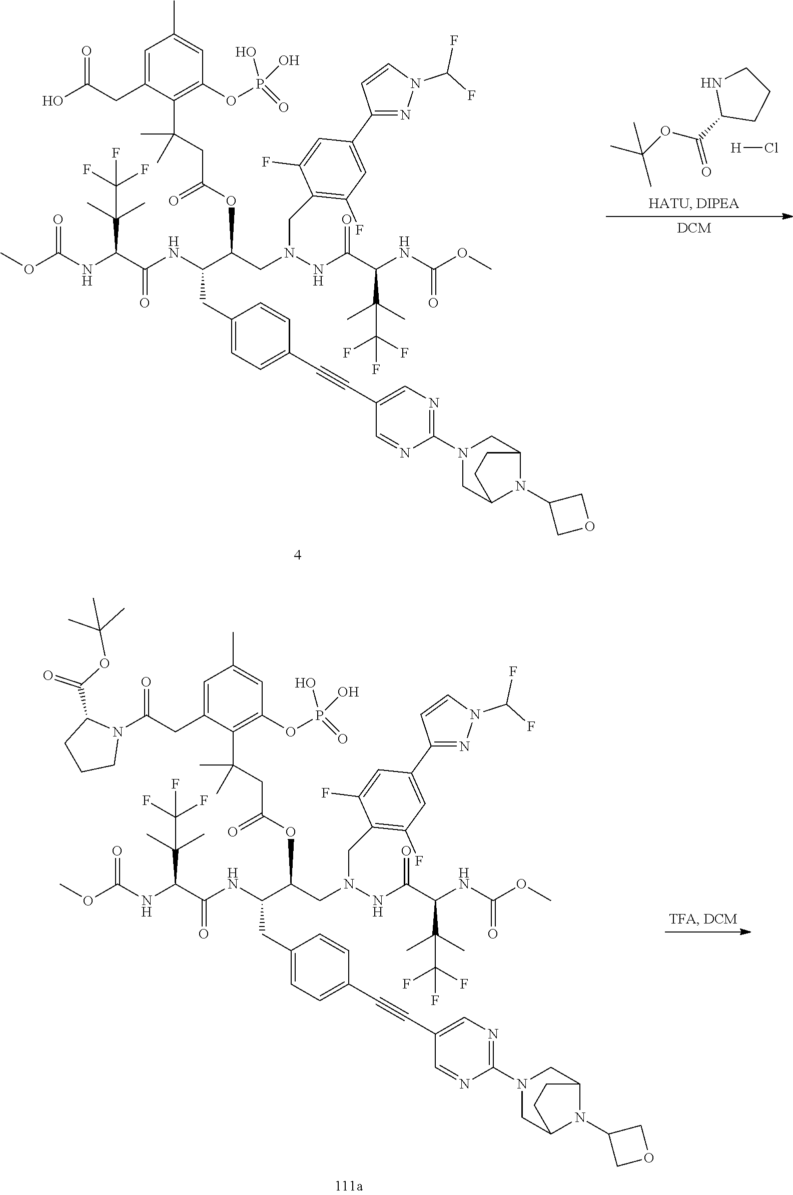

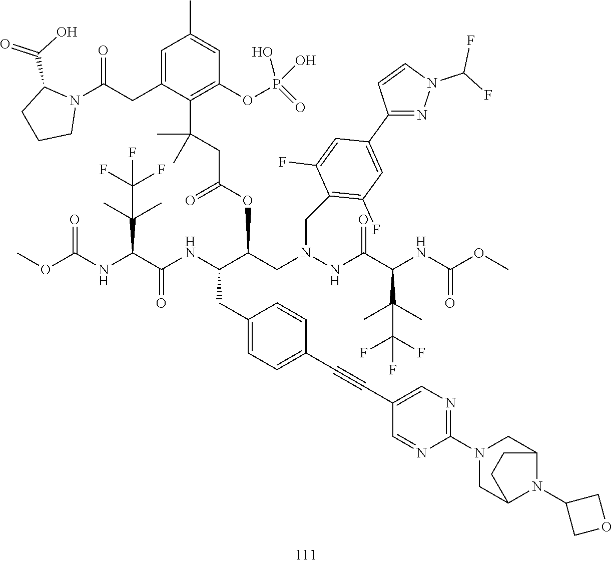 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@@H]2C(=O)O)cc(C)cc1OP(=O)(O)O)C(C)(C)C(F)(F)F]

### Example 111

(2-(2-(4-(((2S,3S)-1-(1-(4-(1-(difluoromethyl)-1H-pyrazol-3-yl)-2,6-difluorobenzyl)-2-((S)-4,4,4-trifluoro-2-((methoxycarbonyl)amino)-3,3-dimethylbutanoyl)hydrazineyl)-4-(4-((2-(8-(oxetan-3-yl)-3,8-diazabicyclo[3.2.1]octan-3-yl)pyrimidin-5-yl)ethynyl)phenyl)-3-((S)-4,4,4-trifluoro-2-((methoxycarbonyl)amino)-3,3-dimethylbutanamido)butan-2-yl)oxy)-2-methyl-4-oxobutan-2-yl)-5-methyl-3-(phosphonooxy)phenyl)acetyl)-D-proline 111 was synthesized using the method described in Example 66, substituting tert-butyl (2R)-pyrrolidine-2-carboxylate hydrochloride for di-tert-butyl L-glutamate hydrochloride. MS (m/z): 791.20 [M+2H]²⁺. ¹H NMR (400 MHz, Methanol-d4) δ 8.54 (s, 2H), 8.12 (d, J=2.8 Hz, 1H), 7.72-7.38 (m), 7.34-7.23 (m), 7.14-7.08 (m), 6.99-6.87 (m), 6.76-6.64 (m), 6.58-6.46 (m), 5.02-4.94 (m), 4.81-4.74 (m), 4.62-4.50 (m), 4.50-4.44 (m), 4.42-4.26 (m), 4.25-4.12 (m), 4.00-3.90 (m), 3.82-3.71 (m), 3.71-3.60 (m), 3.58-3.47 (m), 3.47-3.39 (m), 3.07-2.97 (m), 2.92-2.71 (m), 2.33-2.19 (m), 2.13-1.95 (m), 1.82-1.70 (m), 1.65-1.59 (m), 1.26-1.12 (m), 1.06-0.94 (m). ¹⁹F NMR (376 MHz, Methanol-d4) δ −77.11-−77.27 (m), −77.71-−77.90 (m), −96.82-−96.89 (m), −96.98-−97.05 (m), −114.13-−114.70 (m).

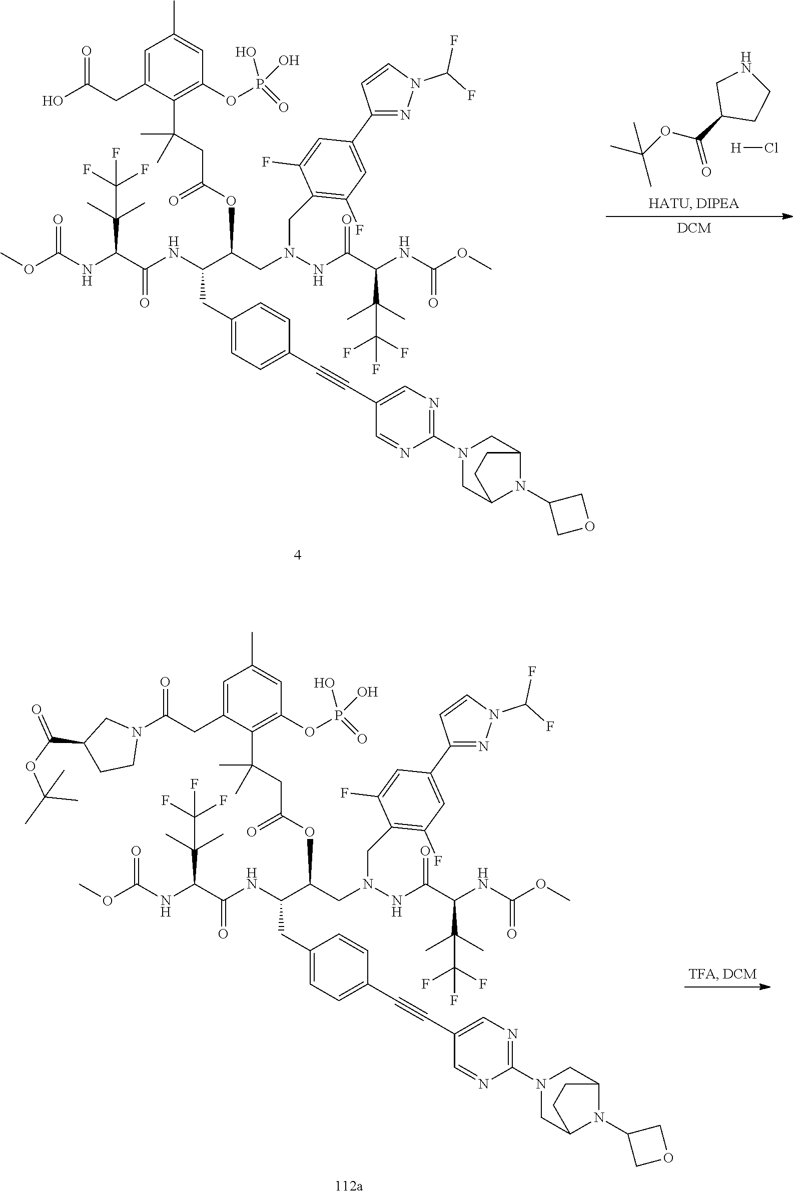

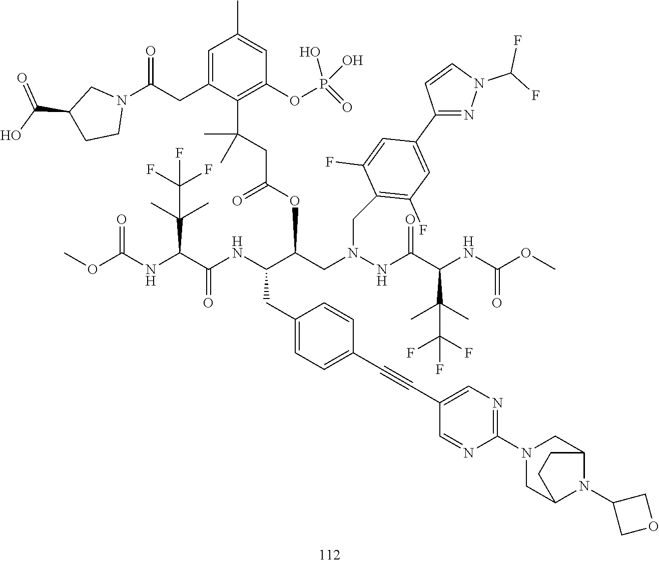 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CC[C@@H](C(=O)O)C2)cc(C)cc1OP(=O)(O)O)C(C)(C)C(F)(F)F]

### Example 112

(3R)-1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)pyrrolidine-3-carboxylic acid 112 was synthesized using the method described in Example 66, substituting tert-butyl (R)-pyrrolidine-3-carboxylate hydrochloride for di-tert-butyl L-glutamate hydrochloride. MS (m/z): 791.20 [M+2H]²⁺. ¹H NMR (400 MHz, Methanol-d4) δ 8.55 (s, 2H), 8.12 (d, J=2.7 Hz, 1H), 7.72-7.39 (m), 7.33-7.23 (m), 7.14-7.08 (m), 6.99-6.86 (m), 6.62 (s, 1H), 5.02-4.93 (m), 4.81-4.74 (m), 4.59-4.47 (m), 4.41-4.27 (m), 4.25-4.13 (m), 3.98-3.88 (m), 3.86-3.77 (m), 3.77-3.72 (m), 3.72-3.66 (m), 3.66-3.56 (m), 3.56-3.46 (m), 3.43-3.37 (m), 3.26-3.09 (m), 3.01-2.85 (m), 2.80-2.72 (m), 2.34-2.13 (m), 2.11-1.95 (m), 1.76-1.70 (m), 1.63-1.62 (m), 1.25-1.11 (m), 1.02 (s, 3H). ¹⁹F NMR (376 MHz, Methanol-d4) δ −77.17 (s), −77.76-−77.78 (m), −96.87 (d, J=8.3 Hz), −97.03 (d, J=8.3 Hz), −114.40 (s).

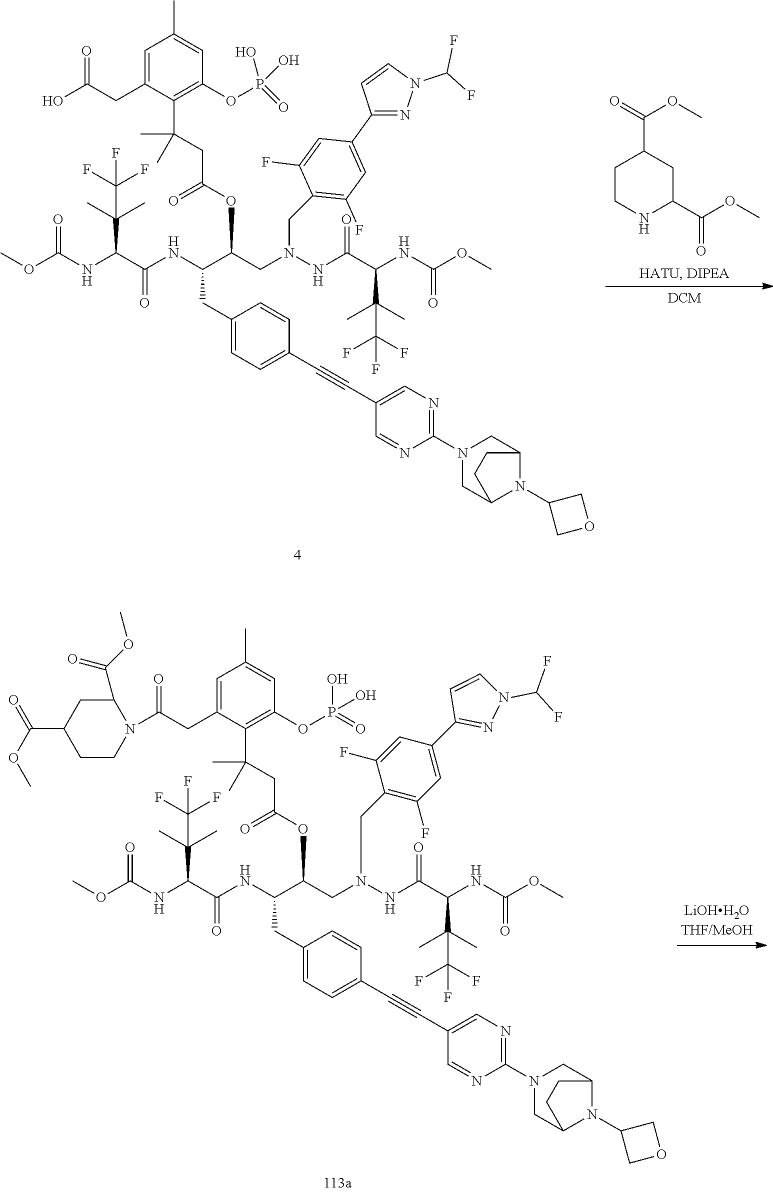 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)O)cc(C)cc1OP(=O)(O)O)C(C)(C)C(F)(F)F ; COC(=O)C1CCNC(C(=O)OC)C1 ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC(C(=O)OC)CC2C(=O)OC)cc(C)cc1OP(=O)(O)O)C(C)(C)C(F)(F)F]

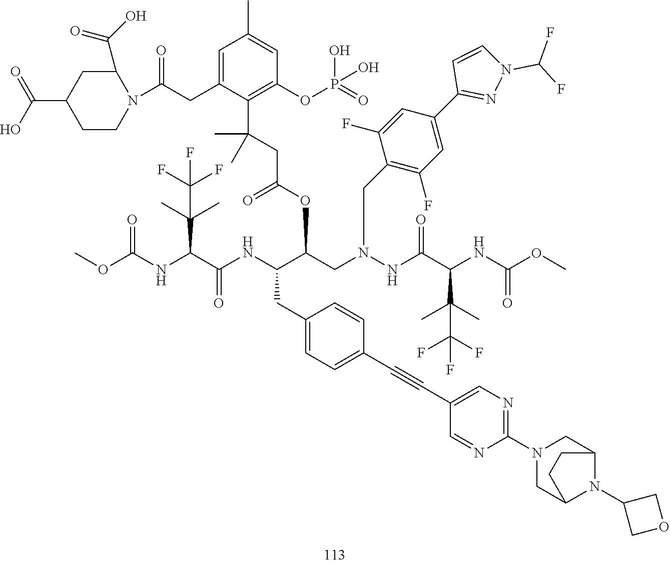 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC(C(=O)O)CC2C(=O)O)cc(C)cc1OP(=O)(O)O)C(C)(C)C(F)(F)F]

### Example 113

1-(2-(2-((5S,10S)-8-(4-(1-(difluoromethyl)-1H-pyrazol-3-yl)-2,6-difluorobenzyl)-14-methyl-10-((1S)-2-(4-((2-(8-(oxetan-3-yl)-3,8-diazabicyclo[3.2.1]octan-3-yl)pyrimidin-5-yl)ethynyl)phenyl)-1-((S)-4,4,4-trifluoro-2-((methoxycarbonyl)amino)-3,3-dimethylbutanamido)ethyl)-3,6,12-trioxo-5-(1,1,1-trifluoro-2-methylpropan-2-yl)-2,11-dioxa-4,7,8-triazapentadecan-14-yl)-5-methyl-3-(phosphonooxy)phenyl)acetyl)piperidine-2,4-dicarboxylic acid 113 was synthesized using the method described in Example 67, substituting dimethyl piperidine-2,4-dicarboxylate for dimethyl piperidine-3,5-dicarboxylate. MS (m/z): 820.20 [M+2H]²⁺. ¹H NMR (400 MHz, Methanol-d4) δ 8.57-8.52 (m), 8.15-8.09 (m), 7.72-7.38 (m), 7.33-7.23 (m), 7.18-7.06 (m), 6.99-6.88 (m), 6.75-6.57 (m), 5.05-4.93 (m), 4.81-4.72 (m), 4.59-4.45 (m), 4.43-4.26 (m), 4.26-4.12 (m), 4.12-3.99 (m), 3.99-3.90 (m), 3.82-3.66 (m), 3.56-3.46 (m), 3.03-2.85 (m), 2.85-2.67 (m), 2.60-2.51 (m), 2.37-2.20 (m), 2.12-1.95 (m), 1.81-1.69 (m), 1.66-1.58 (m), 1.25-1.11 (m), 1.10-0.95 (m). ¹⁹F NMR (376 MHz, Methanol-d4) δ −77.12-−77.28 (m), −77.66-−77.84 (m), −96.83-−96.93 (m), −97.00-−97.08 (m), −114.22-−114.72 (m).

### 3. Biological Assays

### MT-4 HIV Wild Type Virus Infection Assay (IIIB Virus)

Test compounds and controls were serially diluted and spotted in replicate into 384 well black assay plates via acoustic transfer (Echo). MT-4 cells were grown in batch, centrifuged and resuspended into fresh CCM media (RPMI w/10% FBS, 1% PS) at 2×10⁶ cells/ml. MT-4 cells were acutely infected with HIV-1 IIIB strain. The size of each infection mix was scaled by the number of sample plates to be tested. Each infection mix was transferred into 5 mL closed tubes and nutated rapidly on a shaker at 37° C. incubator for 1 hour. The infection mixes were then diluted 25× in fresh cell culture media and then added to assay plates at 40 μL per well using a ViaFlo 384 pipettor. After 5 day incubation at 37° C. in a CO₂ incubator, assay plates were processed with Cell-titer glo reagent using a ViaFlo 384 with an addition/mixing program. Plates were read immediately on Envision reader. Assay signals were plotted and dose response curves generated to determine individual compound EC50s. Results are reported in Table 1 below.

TABLE 1

| Compound | EC₅₀ (nM) |
|---|---|
| 1 | 7.6 |
| 2 | 2.1 |
| 3 | 96.6 |
| 4 | 20.7 |
| 5 | 73.7 |
| 6 | 20.7 |
| 7 | 23.2 |
| 8 | 229.5 |
| 9 | 241.2 |
| 10 | 417.9 |
| 11 | 577.3 |
| 12 | 23.4 |
| 13 | 222.6 |
| 14 | 177.7 |
| 15 | 162.1 |
| 16 | 62.2 |
| 17 | 169.6 |
| 18 | 251 |
| 19 | 38.9 |
| 20 | 0.7 |
| 21 | 3.6 |
| 22 | 9.9 |
| 23 | 203.7 |
| 24 | 130.4 |
| 25 | 62.7 |
| 26 | 96.9 |
| 27 | 172.7 |
| 28 | 306.8 |
| 29 | 212.7 |
| 30 | 197.1 |
| 31 | 276.5 |
| 32 | 136.9 |
| 33 | 65.7 |
| 34 | 179.4 |
| 35 | 65.7 |
| 36 | 97.4 |
| 37 | 41.8 |
| 38 | 304.8 |
| 39 | 64.4 |
| 40 | 40.5 |
| 41 | 43.5 |
| 42 | 49.1 |
| 43 | 85.9 |
| 44 | 527.8 |
| 45 | 208.8 |
| 46 | 190.1 |
| 47 | 593 |
| 48 | 201 |
| 49 | 223.5 |
| 50 | 1716.9 |
| 51 | 636.1 |
| 52 | 67.2 |
| 53 | 113 |
| 54 | 94 |
| 55 | 3478.4 |
| 56 | 254.4 |
| 57 | 289.7 |
| 58 | 370.5 |
| 59 | 657.8 |
| 60 | 217.9 |
| 61 | 1885.2 |
| 62 | 279.9 |
| 63 | 243.5 |
| 64 | 11.1 |
| 65 | 12.6 |
| 66 | 163.5 |
| 67 | 204.7 |
| 68 | 5.9 |
| 69 | 3.5 |
| 70 | 6.6 |
| 71 | 3.9 |
| 72 | 5.4 |
| 73 | 4.6 |
| 74 | 193.7 |
| 75 | 8.1 |
| 76 | 332.8 |
| 77 | 8.3 |
| 78 | 3.1 |
| 79 | 644.8 |
| 80 | 735.2 |
| 81 | 627.1 |
| 82 | 334.8 |
| 83 | 1278.3 |
| 84 | 3.9 |
| 85 | 2.4 |
| 86 | 4.1 |
| 87 | 3.6 |
| 88 | 42.8 |
| 89 | 114.6 |
| 90 | 101.1 |
| 91 | 60 |
| 92 | 37.1 |
| 93 | 55.8 |
| 94 | 59 |
| 95 | 99.1 |
| 96 | 558.9 |
| 97 | 204.1 |
| 98 | 64.6 |
| 99 | 372.7 |
| 100 | 42.4 |
| 101 | 60.6 |
| 102 | 54.1 |
| 103 | 131.5 |
| 104 | 17.6 |
| 105 | 217.2 |
| 106 | 761 |
| 107 | 317.3 |
| 108 | 444 |
| 109 | 90.5 |
| 110 | 138.1 |
| 111 | 451.8 |
| 112 | 224.2 |
| 113 | 341 |

### Pharmacokinetic Profiling

Test compounds were formulated in suspension formulations (0.5% hydroxypropyl methylcellulose (HPMC) in DI water) and administered orally at 10 mg-eq./kg to a dosing group consisting of three non-naïve male beagle dogs. Intermediate A was formulated in a suspension formulation (1% HPMC and 0.05% Tween 20 in DI water) and administered orally at 10 mg-eq./kg to a dosing group consisting of three non-naïve male beagle dogs. The animals were fasted overnight prior to dose administration and up to four hours after dosing. The test articles were administrated by oral gavage at a dose volume of 5 ml/kg. To stimulate gastric secretion, each animal received a single 6-μg/kg intramuscular injection of pentagastrin approximately 30 minutes prior to test article administration. The intramuscular dose was administered in a thigh muscle using a needle and syringe.

Blood samples were collected at predose and 0.25, 0.5, 1, 2, 4, 6, 8, 12, 24 and 48 hours post dose into K2EDTA tubes and stored on wet ice until processed. Whole blood was processed to plasma by centrifugation (3500 rpm for 10 minutes at 5° C.) within 30 minutes of collection. Plasma samples were transferred into Micronic 96 well tubes and stored at −80° C. as soon as possible. Following the measurement of concentration of the test compounds in plasma, maximum observed concentration (Cₘₐₓ) and area under the curve from time of dosing to last measured concentration at 48 hours post dose (AUCₗₐₛₜ) were calculated and are reported in Table 2 below.

TABLE 2

| | | Intermediate |
|---|---|---|
| | Intermediate | A AUCₗₐₛₜ |
| Compound | A Cₘₐₓ (nM) | (nM · h) |
| Intermediate | 44.2 | 775 |
| A | | |
| 4 | 461 | 3610 |
| 16 | 575 | 7590 |
| 20 | 350 | 3860 |
| 21 | 426 | 4840 |
| 23 | 385 | 3870 |
| 24 | 414 | 4060 |
| 32 | 646 | 7500 |
| 33 | 657 | 6930 |
| 49 | 138 | 1470 |
| 66 | 515 | 6010 |
| 68 | 365 | 5610 |
| 71 | 357 | 7510 |
| 74 | 760 | 6940 |
| 79 | 698 | 9870 |
| 104 | 420 | 3970 |

### BiALP

Fosamprenavir and its parent compound (amprenavir) were purchased from Millipore Sigma (St. Louis, MO). Test compounds, Intermediate A, fosamprenavir, and amprenavir were each separately dissolved in dimethyl sulfoxide (DMSO) at a concentration of 10 mM to form stock solutions.

All other chemicals were purchased from Millipore Sigma (St. Louis, MO) and Thermo Fisher Scientific (Waltham, MA). The chemicals included purified bovine intestinal alkaline phosphatase as a lyophilized powder (specific activity: 101 diethanolamine units/mg solid). Internal Standard/Quench (IS/Q) was 100 nM labetalol in 99:1 (v/v) acetonitrile/formic acid.

Incubations were performed with Tris buffer alone (0.1 M Tris-HCl pH 7.8 containing 1 mM MgCl₂ and 1 mM ZnCl₂) or the same Tris buffer further containing 100 ng/mL iALP (0.01 units/mL). Triplicate 100 μL aliquots of Tris buffer±iALP were warmed to 37° C. and the reactions were initiated by the addition of the test compound or fosamprenavir to obtain final substrate concentrations of 2 μM. Incubations were then continued at 37° C. At 60 minutes, reactions were terminated by the addition of 200 μl of IS/Q and mixed by pipette. After quenching, the plates were centrifuged at 4713×gₐᵥ at 4° C. for 20 minutes. Aliquots (150 μL) of the supernatants were transferred to fresh plates containing 150 μL of water. Aliquots (15 μL) of the diluted supernatants were analyzed by mass spectrometry as described below.

Quantification of the test compounds and fosamprenavir was performed by analyte/internal standard peak area ratios (PARs) with comparison to 2 μM Intermediate A and amprenavir, respectively. The LC instrumentation consisted of a Thermo Scientific Vanquish pump and a Thermo Hypersil Gold column (1.9 μm particle size, 2.1×50 mm). Mobile phases were: A: water 99.9% (v/v) containing 0.1% (v/v) formic acid, and B: acetonitrile 99.9% (v/v) containing 0.1% (v/v) formic acid, pumped at 0.5 mL/min. Elution of test compounds, fosamprenavir, Intermediate A, and amprenavir was achieved by a series of linear gradients over 2.83 min followed by re-equilibration for 1.17 min between injections. The MS instrument was a Thermo Q Exactive™ Plus operating in positive ionization mode with mass tolerance of 5 ppm and calibrated on a twice weekly basis. Results are reported in Table 3 below.

TABLE 3

| | | | biALP |
|---|---|---|---|
| | | [Intermediate | Intermediate |
| | | A] or | A or |
| | [Compound] | [Amprenavir] | [Amprenavir] |
| Compound | (nM) | (nM) | (% of total) |
| Fosamprenavir | 1605 | 226 | 12.5 |
| 1 | 1318.1 | 350.1 | 21 |
| 2 | 1359.6 | 329.5 | 19.5 |
| 3 | 1197.7 | 109.8 | 8.4 |
| 4 | 1627.4 | 215.7 | 11.7 |
| 5 | 1638.1 | 234.3 | 12.5 |
| 6 | 1304.1 | 306.6 | 19 |
| 7 | 1590.8 | 164.7 | 9.4 |
| 8 | 1709.4 | 157.3 | 8.4 |
| 9 | 2004.9 | 36.5 | 1.8 |
| 10 | 1868.8 | 95.5 | 4.9 |
| 11 | 1473 | 3.7 | 0.3 |
| 12 | | | |
| 13 | 1660.2 | 1.8 | 0.1 |
| 14 | 2276.6 | 50.3 | 2.2 |
| 15 | 2690.4 | 47.4 | 1.7 |
| 16 | 1784.3 | 239.6 | 11.8 |
| 17 | 1725.9 | 175.6 | 9.2 |
| 18 | 2177.4 | 8.7 | 0.4 |
| 19 | 1405.7 | 317.9 | 18.4 |
| 20 | 1351.2 | 1276.2 | 48.6 |
| 21 | 1281.9 | 1034.6 | 44.7 |
| 22 | 1221.6 | 1468.7 | 54.6 |
| 23 | 2107.1 | 82.9 | 3.8 |
| 24 | 1717.9 | 256.7 | 13 |
| 25 | 2023.7 | 45.6 | 2.2 |
| 26 | 1764 | 38 | 2.1 |
| 27 | 2082.4 | 197.3 | 8.7 |
| 28 | 2478.7 | 0 | 0 |
| 29 | 1878.7 | 61.5 | 3.2 |
| 30 | 1521.8 | 50.8 | 3.2 |
| 31 | 2125.6 | 3.2 | 0.2 |
| 32 | 1435.5 | 353.9 | 19.8 |
| 33 | 1487.1 | 235.6 | 13.7 |
| 34 | 2238.2 | 4.9 | 0.2 |
| 35 | 2145.2 | 19.2 | 0.9 |
| 36 | 1383.3 | 267 | 16.2 |
| 37 | 1518.9 | 198.6 | 11.6 |
| 38 | 1920.9 | 28.2 | 1.4 |
| 39 | 1310.3 | 272.1 | 17.2 |
| 40 | 1458.8 | 173.9 | 10.7 |
| 41 | 2074.8 | 174.9 | 7.8 |
| 42 | 1335 | 173.6 | 11.5 |
| 43 | 1661.5 | 91.5 | 5.2 |
| 44 | 1835.9 | 181.7 | 9 |
| 45 | 1787.4 | 209.8 | 10.5 |
| 46 | 1864.7 | 0 | 0 |
| 47 | 1950.3 | 0 | 0 |
| 48 | 2071.6 | 14.2 | 0.7 |
| 49 | 2175 | 89.6 | 4 |
| 50 | 2228.1 | 0.3 | 0 |
| 51 | 2331.5 | 0.8 | 0 |
| 52 | 2131.6 | 7.6 | 0.4 |
| 53 | 2028.9 | 109 | 5.1 |
| 54 | 1661.6 | 0 | 0 |
| 55 | 1659.2 | 4.7 | 0.3 |
| 56 | 1576.7 | 5.9 | 0.4 |
| 57 | 1612.8 | 9.7 | 0.6 |
| 58 | 1573.2 | 3.5 | 0.2 |
| 59 | 1520.4 | 1.3 | 0.1 |
| 60 | 1457.7 | 19 | 1.3 |
| 61 | 1611.1 | 3.3 | 0.2 |
| 62 | 2213.4 | 0 | 0 |
| 63 | 1778.4 | 41.6 | 2.3 |
| 64 | 1271 | 395 | 23.7 |
| 65 | 1224.6 | 553.4 | 31.1 |
| 66 | 1908 | 197.1 | 9.4 |
| 67 | 1747.2 | 175.4 | 9.1 |
| 68 | 1478.2 | 584 | 28.3 |
| 69 | 1578.5 | 335.6 | 17.5 |
| 70 | 1164.8 | 828.5 | 41.6 |
| 71 | 1036.2 | 924 | 47.1 |
| 72 | 827.8 | 451.8 | 35.3 |
| 73 | 1660.4 | 504 | 23.3 |
| 74 | 2044.2 | 96.7 | 4.5 |
| 75 | 1420.1 | 927.5 | 39.5 |
| 76 | 1777 | 82.8 | 4.5 |
| 77 | 2259 | 422.7 | 15.8 |
| 78 | 2070.6 | 530.3 | 20.4 |
| 79 | 2057.5 | 7.1 | 0.3 |
| 80 | 2507.9 | 0 | 0 |
| 81 | 2385.2 | 0 | 0 |
| 82 | 1224.3 | 1.6 | 0.1 |
| 83 | 1460.2 | 0 | 0 |
| 84 | 1594.8 | 178.9 | 10.1 |
| 85 | 1466.1 | 176.7 | 10.8 |
| 86 | 1890 | 112.9 | 5.6 |
| 87 | 1161.5 | 1378 | 54.3 |
| 88 | 1726.6 | 109.8 | 6 |
| 89 | 1704.6 | 101.7 | 5.6 |
| 90 | 1746 | 68.6 | 3.8 |
| 91 | 2153.3 | 89 | 4 |
| 92 | 1771.4 | 306.2 | 14.7 |
| 93 | 1917.5 | 44.9 | 2.3 |
| 94 | 1912 | 18.1 | 0.9 |
| 95 | 1927.5 | 28.6 | 1.5 |
| 96 | 2114.6 | 37.1 | 1.7 |
| 97 | 1772.9 | 5.3 | 0.3 |
| 98 | 1320.1 | 66.2 | 4.8 |
| 99 | 1928.8 | 38.8 | 2 |
| 100 | 1103.4 | 3.2 | 0.3 |
| 101 | 1688.4 | 35.5 | 2.1 |
| 102 | 1966.3 | 59.1 | 2.9 |
| 103 | 1589 | 33.5 | 2.1 |
| 104 | 1622.8 | 40 | 2.4 |
| 105 | 1626.1 | 120.8 | 6.9 |
| 106 | 1588.2 | 129.5 | 7.5 |
| 107 | 1468.1 | 172.2 | 10.5 |
| 108 | 2296.1 | 39 | 1.7 |
| 109 | 1951.1 | 65.9 | 3.3 |
| 110 | 1852.3 | 10.1 | 0.5 |
| 111 | 1803.5 | 7.6 | 0.4 |
| 112 | 1708.7 | 40 | 2.3 |
| 113 | 1948 | 19.4 | 1 |

### Stability

Reagents. Stock solutions of test compounds in dimethyl sulfoxide (DMSO) having a final concentration of 10 mM were prepared and used in all experiments. Sekisui XenoTech (Kansas City, KS) provided pooled intestinal S9 fractions. All other chemicals were purchased from Sigma-Aldrich (St. Louis, MO) or VWR (West Chester, PA). Internal Standard/Quench (IS/Q) used to stop reactions was 100 nM labetalol in (by volume) formic acid (1%) and acetonitrile (99%).

Intestinal S9 Assay: For each test compound, an aliquot of the test compound was added to S9 stock diluted with TRIS buffer (100 mM TRIS, 1 mM ZnCl₂, 1 mM MgCl₂), pH 7.4, to obtain an incubation protein concentration of 1.0 mg/mL. The incubation was initiated by the addition of the substrate to the S9 reaction mixture to a final concentration of 2 μM. At 0, 10, 20, 30, 60 and 120 min, 25 μL aliquots of the reaction mixture were transferred to plates containing 225 μl of IS/Q solution. After quenching, the plates were centrifuged at 3000×g for 30 minutes. 150 μL aliquots of each supernatant were transferred to new plates and 150 μL water was added. Aliquots (10 μL) of the prepared samples were analyzed on a Thermo Q-Exactive mass spectrometer as described below.

PBS Stability Assay: For each test compound, an aliquot of the test compound was added to phosphate buffered saline (137 mM NaCl, 2.7 mM KCl, 10 mM phosphate buffer), pH 7.4. The incubation was initiated by the addition of the test compound to PBS to a final concentration of 2 μM. At 0, 10, 20, 30, 60 and 120 min, 25 μL aliquots of the incubation mixture were transferred to plates containing 225 μl of IS/Q solution. After quenching, the plates were centrifuged at 3000×g for 30 minutes. 150 μL aliquots of each supernatant were transferred to new plates and 150 μL water was added. Aliquots (10 μL) of the prepared samples were analyzed on a Thermo Q-Exactive mass spectrometer as described below.

Liquid Chromatography-Mass Spectrometry: Quantification of test compounds and control substrate was performed by analyte/internal standard peak area ratio (PAR) values measured on a Thermo Q-Exactive mass spectrometer coupled to a Dionex UltiMate 3000 HPLC with a Leap Technologies HTC PAL autosampler. The column used for analysis was a Thermo Scientific Hypersil GOLD (1.9 μm particle size, 50×2.1 mm). Mobile phase A consisted of 0.1% (v/v) formic acid in water. Mobile phase B consisted of 0.1% (v/v) formic acid in acetonitrile. Elution of analytes was achieved by a series of linear gradients varying the proportions of A and B. The mass spectrometer was calibrated on a weekly basis and mass tolerance of 5 ppm was used.

Data Analysis. Metabolic stabilities were determined by measuring the rates of disappearance of test compound and positive control substrate. Data (% of substrate remaining) were plotted on a semi-log scale and fitted using an exponential decline model:

$$\\ {C}_{t} = {C}_{0} \times {e}^{{}_{} - ln 2 / T 1 / 2 \times t}$$

where
Cₜ% of substrate remaining at time=t
C₀% of substrate remaining at time=0
t time
T½ half-life
The Half-life (T½) is determined by the following equation:

$$\\ T 1 / 2 = - ln 0.5 / k = 0.693 / k$$

Assuming a first-order reaction, the slope (k) is extrapolated from the aforementioned plotted data.

Results are shown in Table 4 below.

TABLE 4

| | Human | Dog | Human | Dog | |
|---|---|---|---|---|---|
| | GI S9 | GI S9 | GI S9 | GI S9 | PBS |
| | T½ | T½ | [Intermediate | [Intermediate | T½ |
| Compound | (min) | (min) | A] (nM), % | A] (nM), % | (min) |
| 1 | | | | | 524.5 |
| 2 | | | | | 391.1 |
| 3 | 118.7 | 41.1 | | | 251.6 |
| 4 | 464.3 | 101.4 | 210.8, 11.1 | 669, 40.2 | 565.8 |
| 5 | 697.4 | 44.4 | 291, 15.8 | 1691.8, 82.4 | 457.2 |
| 6 | 154.2 | 23.6 | 363.6, 28.2 | 1236, 93.8 | 655.8 |
| 7 | 136.1 | 27.4 | 404.7, 31.3 | 1224.2, 91 | 523.5 |
| 8 | | | | | 426.8 |
| 9 | 535.5 | 68.3 | | | 623 |
| 10 | 553.2 | 254.3 | 86.8, 7.3 | 228.1, 18.4 | 595.2 |
| 11 | 789 | 789 | | | |
| 12 | | | | | 295 |
| 13 | | | | | 118.7 |
| 14 | 604.6 | 401.1 | 58.5, 3.9 | 210.8, 13.8 | 334.5 |
| 15 | 537.2 | 236.9 | 71.3, 5.2 | 230, 17 | 288.1 |
| 16 | 287 | 52.2 | 210.4, 15.2 | 890, 66 | 311.4 |
| 17 | | | | | 262.5 |
| 18 | 488.4 | 364.7 | 37.6, 3 | 148.6, 10.9 | |
| 19 | 229.9 | 18 | 323.6, 22.1 | 1045.1, 94.7 | |
| 20 | | | | | 390.1 |
| 21 | | | | | 789 |
| 22 | | | | | 373.3 |
| 23 | | | | | 703.8 |
| 24 | 227.1 | 134.2 | | | 789 |
| 25 | | | | | 82.4 |
| 26 | | | | | 146.2 |
| 27 | 147.8 | 109.5 | | | 384.5 |
| 28 | 789 | 685.6 | | | 789 |
| 29 | 198.3 | 49.3 | | | 517 |
| 30 | 246.8 | 70.3 | | | 513.9 |
| 31 | 494.6 | 222.3 | 101.1, 8.1 | 385.4, 29.1 | |
| 32 | 231.9 | 60.1 | 139.5, 12.2 | 598.3, 54.3 | 310.7 |
| 33 | 223.4 | 90.4 | 122.8, 10.5 | 382.3, 36.7 | 342.5 |
| 34 | | | | | 304.8 |
| 35 | 192.5 | 14.1 | | | 196.4 |
| 36 | | | | | 656.3 |
| 37 | | | | | 439 |
| 38 | 469.3 | 391.5 | 47.1, 3.7 | 103.4, 8.1 | 309.6 |
| 39 | 192.4 | 19.3 | 268, 20.8 | 1060.3, 94.6 | |
| 40 | 285.6 | 32.2 | 264.3, 19.1 | 998.3, 87.6 | |
| 41 | | | | | 301.1 |
| 42 | | | | | 361.5 |
| 43 | | | | | 289 |
| 44 | | | | | 280.3 |
| 45 | 373.6 | 59.6 | | | 284.5 |
| 46 | 444.9 | 305.1 | 17.9, 1.5 | 94.1, 7.5 | |
| 47 | 383.2 | 440.3 | 8.6, 0.7 | 12.5, 1 | |
| 48 | | | | | 285.4 |
| 49 | 330 | 155.5 | 217.9, 17.2 | 314.3, 24.7 | 339.5 |
| 50 | | | | | 495.7 |
| 51 | 479 | 497.4 | | | 402.6 |
| 52 | 304 | 155.3 | | | 355.4 |
| 53 | 203.8 | 144.6 | 159.5, 12.9 | 281.4, 22.4 | 585.4 |
| 54 | 772.7 | 566.6 | 91, 5.8 | 145.9, 9 | |
| 55 | 764.2 | 717.9 | 0, 0 | 2.1, 0.1 | |
| 56 | 789 | 413.2 | 53.1, 3.7 | 107.7, 7.6 | |
| 57 | 657.1 | 431.6 | 42.7, 2.8 | 97.1, 6.3 | |
| 58 | 727 | 433.7 | 23.7, 2 | 61.3, 5.1 | |
| 59 | 789 | 400.1 | 42.3, 3.2 | 45.1, 3.8 | |
| 60 | 720.3 | 348.5 | 37.5, 2.7 | 55.2, 4.2 | |
| 61 | 702 | 670.9 | 4.1, 0.3 | 13.5, 0.9 | |
| 62 | 789 | 789 | 22.2, 1.8 | 38.3, 3.1 | |
| 63 | 789 | 382.1 | 103.4, 7.2 | 328.4, 21.7 | |
| 64 | | | | | 505.9 |
| 65 | | | | | 668.9 |
| 66 | 191.7 | 54.7 | 367.6, 26 | 965.9, 68.6 | 291.4 |
| 67 | | | | | 259.4 |
| 68 | | | | | 376.1 |
| 69 | | | | | 784.2 |
| 70 | | | | | 316.3 |
| 71 | | | | | 471.6 |
| 72 | | | | | 367.3 |
| 73 | | | | | 381.5 |
| 74 | 236 | 62 | 214.4, 15.3 | 904.6, 61.1 | 692.4 |
| 75 | | | | | 286.2 |
| 76 | 369.4 | 264.6 | 72.7, 5.7 | 195.9, 14.3 | 293.3 |
| 77 | | | | | |
| 78 | | | | | |
| 79 | 611.7 | 562.5 | 34.4, 2.4 | 71, 4.8 | |
| 80 | | | | | |
| 81 | | | | | |
| 82 | | | | | 402.5 |
| 83 | | | | | 193.1 |
| 84 | | | | | 181.1 |
| 85 | | | | | 136.1 |
| 86 | | | | | 155.2 |
| 87 | | | | | 324.8 |
| 88 | | | | | 369.9 |
| 89 | | | | | 317.7 |
| 90 | | | | | 327.4 |
| 91 | | | | | 389.6 |
| 92 | | | | | 357 |
| 93 | | | | | 166.1 |
| 94 | | | | | 223.2 |
| 95 | | | | | 253.3 |
| 96 | | | | | 259.5 |
| 97 | | | | | 278.7 |
| 98 | | | | | 388.2 |
| 99 | | | | | 446.9 |
| 100 | | | | | 313.2 |
| 101 | | | | | 485.3 |
| 102 | | | | | 467 |
| 103 | | | | | 331.3 |
| 104 | 125.8 | 9 | | | 285 |
| 105 | 558.1 | 290.9 | 75.4, 5.6 | 155.6, 12.2 | 330.9 |
| 106 | | | | | 271.1 |
| 107 | 246.8 | 132.5 | | | 381.4 |
| 108 | 432.3 | 267.6 | | | |
| 109 | 268.7 | 133.9 | | | |
| 110 | 510.7 | 168.6 | | | |
| 111 | 617.7 | 98.4 | | | |
| 112 | 573.3 | 363.3 | 76.4, 5.3 | 146.2, 10.6 | 391.9 |
| 113 | 393.4 | 202.9 | 48.5, 3.9 | 448.7, 31.7 | |

### Solubility in FaSSIF

Buffer Preparation: Fasted State Simulated Intestinal Fluid (FaSSIF): Simulated Intestinal Fluid (SIF) powder containing a complex of Taurocholate and Lecithin (4:1 molar ratio) was obtained from Biorelevant. 0.056 g of SIF powder was added to 25 mL of buffer, adjusted to pH 6.5, containing 0.011 g of NaOH (pellets), 0.099 g of NaH2PO4 (monohydrate), and 0.155 g of NaCl. The buffer was prepared with HPLC H2O. The SIE powder was stirred until completely dissolved and allowed to stand for 2 hours.

Test Compounds: Each test compound was placed in a vial containing approximately 7 mg of material. Aliquots were weighed out for each assay media at each time point to be analyzed. The buffer was added to each vial such that the final dose concentration of 5 mg/mL was achieved. Samples were then vortexed for 5-10 seconds.

Following a 2-hour incubation on a rotary shaker (200 RPM) at ambient temperature (22.1-23.7° C.), the samples were vacuum filtered through a Millipore solubility filter plate with 0.45 μM polycarbonate filter membrane and the filtrates were collected in a 96 well polypropylene plate. The plate was sealed with a pierceable heat seal and analyzed by HPLC-UV. Results are depicted in Table 5.

TABLE 5

| | FaSSIF (pH 6.5) |
|---|---|
| Compound | (μg/mL) |
| 1 | 5065 |
| 2 | 680 |
| 3 | — |
| 4 | 271.7 |
| 5 | <1000 |
| 6 | 3710 |
| 7 | 5490 |
| 8 | 3260 |
| 9 | 2300 |
| 10 | 3120 |
| 11 | 25 |
| 12 | — |
| 13 | — |
| 14 | <1000 |
| 15 | <1000 |
| 16 | 3785 |
| 17 | 4305 |
| 18 | <1000 |
| 19 | 3650 |
| 20 | 220 |
| 21 | 4340 |
| 22 | 4715 |
| 23 | 4542.5 |
| 24 | 4655 |
| 25 | 2410 |
| 26 | 1435 |
| 27 | 4420 |
| 28 | 15 |
| 29 | 4970 |
| 30 | 5370 |
| 31 | 3507.5 |
| 32 | 3400 |
| 33 | 4245 |
| 34 | 4810 |
| 35 | 3555 |
| 36 | 4255 |
| 37 | 4460 |
| 38 | 3505 |
| 39 | 5097.5 |
| 40 | 4907.5 |
| 41 | — |
| 42 | 4020 |
| 43 | 1925 |
| 44 | 4550 |
| 45 | 4305 |
| 46 | 2867.5 |
| 47 | 3620 |
| 48 | 910 |
| 49 | 1220 |
| 50 | <1000 |
| 51 | <1000 |
| 52 | <1000 |
| 53 | <1000 |
| 54 | 3080 |
| 55 | <1000 |
| 56 | 2395 |
| 57 | 4095 |
| 58 | 4465 |
| 59 | 3600 |
| 60 | 3485 |
| 61 | 2575 |
| 62 | <1000 |
| 63 | 495 |
| 64 | 165 |
| 65 | 1310 |
| 66 | 3889 |
| 67 | 3165 |
| 68 | 4435 |
| 69 | 4950 |
| 70 | 5570 |
| 71 | 4420 |
| 72 | 4980 |
| 73 | 145 |
| 74 | 3335 |
| 75 | 4170 |
| 76 | 2230 |
| 77 | 2755 |
| 78 | 4390 |
| 79 | 2676.4 |
| 80 | <1000 |
| 81 | <1000 |
| 82 | <1000 |
| 83 | <1000 |
| 84 | — |
| 85 | <1000 |
| 86 | <1000 |
| 87 | 4505 |
| 88 | 4645 |
| 89 | 2390 |
| 90 | 4240 |
| 91 | <1000 |
| 92 | 3665 |
| 93 | <1000 |
| 94 | 1585 |
| 95 | 4265 |
| 96 | 1845 |
| 97 | 4665 |
| 98 | 4285 |
| 99 | 1885 |
| 100 | 4450 |
| 101 | 1525 |
| 102 | 2860 |
| 103 | 80 |
| 104 | 2730 |
| 105 | 3285 |
| 106 | 3920 |
| 107 | 3445 |
| 108 | <1000 |
| 109 | <1000 |
| 110 | 4415 |
| 111 | 4760 |
| 112 | 3790 |
| 113 | 4140 |

1-150. (canceled)

151. A compound selected from: 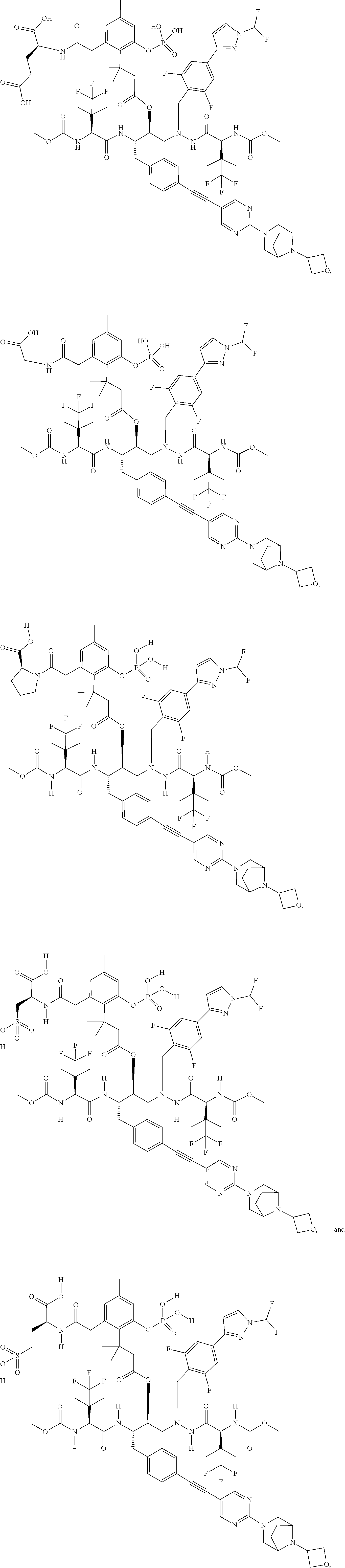
or a pharmaceutically acceptable salt thereof.

152. The compound of claim 151, which is 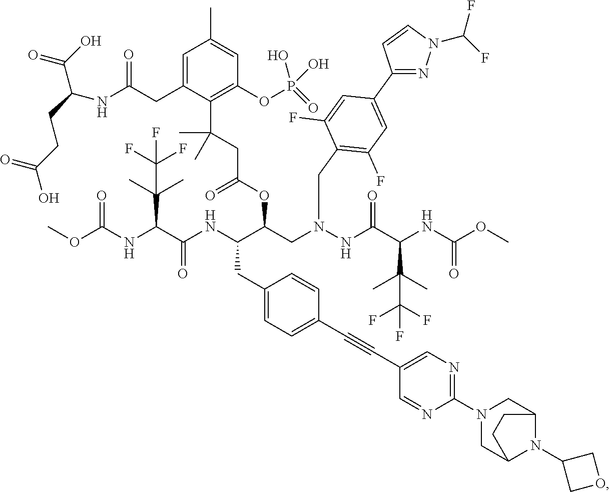 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cc(C)cc1OP(=O)(O)O)C(C)(C)C(F)(F)F]
or a pharmaceutically acceptable salt thereof.

153. The compound of claim 151, which is 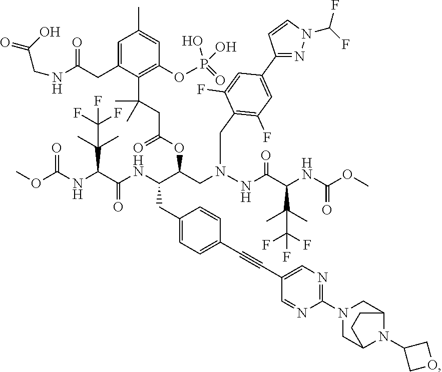 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)O)cc(C)cc1OP(=O)(O)O)C(C)(C)C(F)(F)F]
or a pharmaceutically acceptable salt thereof.

154. The compound of claim 151, which is 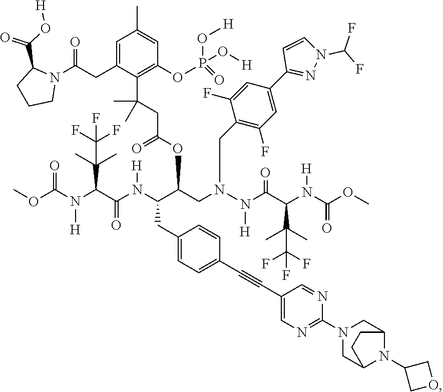 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@H]2C(=O)O)cc(C)cc1OP(=O)(O)O)C(C)(C)C(F)(F)F]
or a pharmaceutically acceptable salt thereof.

155. The compound of claim 151, which is 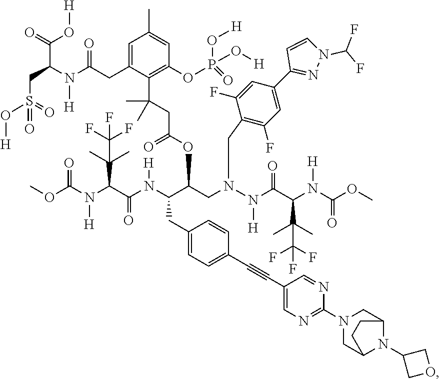 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]
or a pharmaceutically acceptable salt thereof.

156. The compound of claim 151, which is 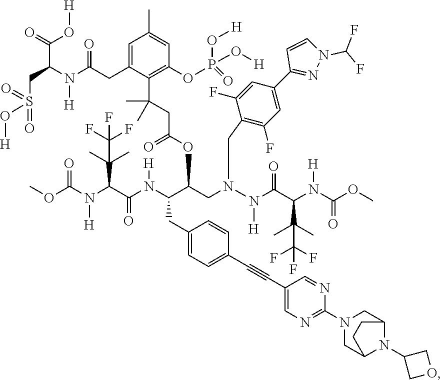 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]
or a pharmaceutically acceptable salt thereof.

157. A compound selected from:

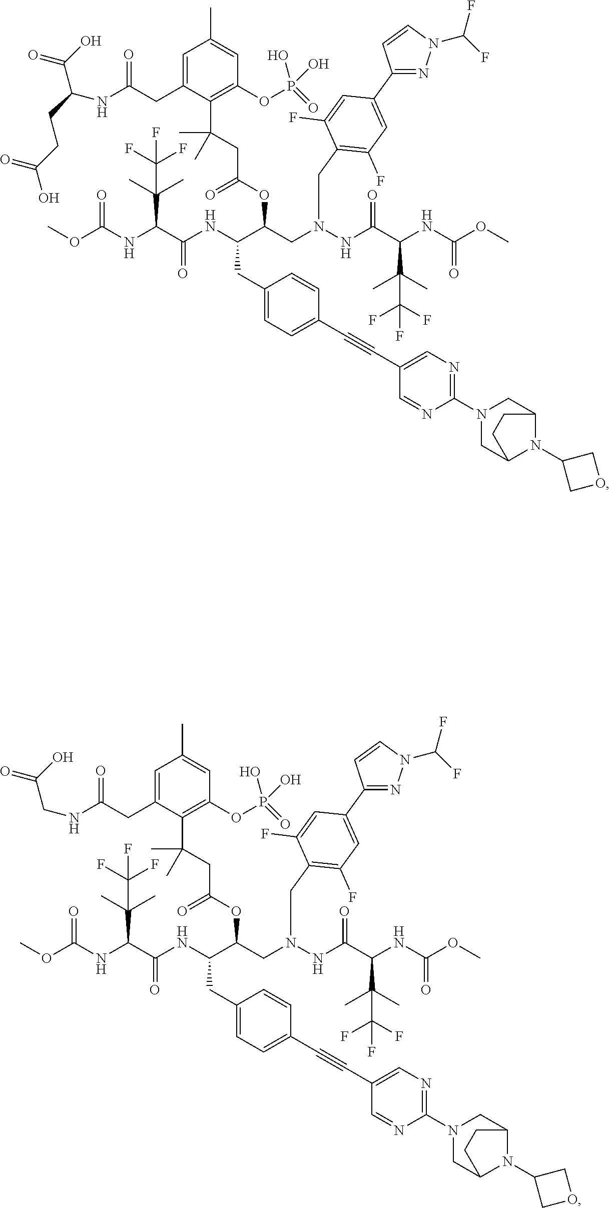 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)O)cc(C)cc1OP(=O)(O)O)C(C)(C)C(F)(F)F]

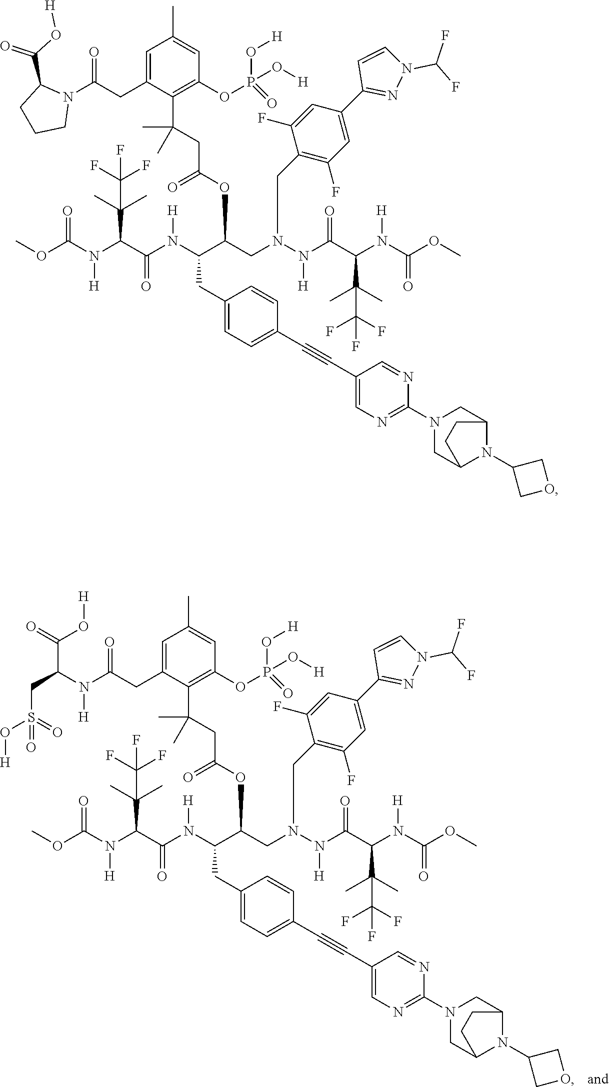 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@H]2C(=O)O)cc(C)cc1OP(=O)(O)O)C(C)(C)C(F)(F)F ; COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

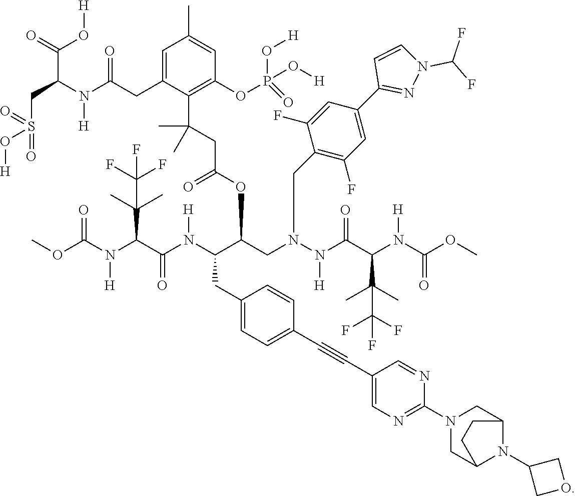 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

158. The compound of claim 157, which is

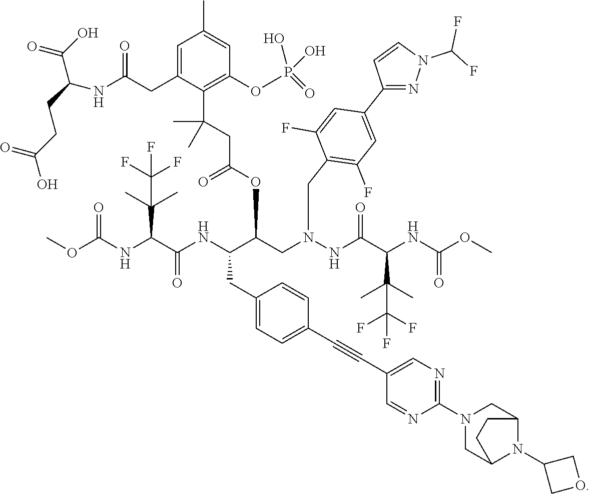 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CCC(=O)O)C(=O)O)cc(C)cc1OP(=O)(O)O)C(C)(C)C(F)(F)F]

159. The compound of claim 157, which is

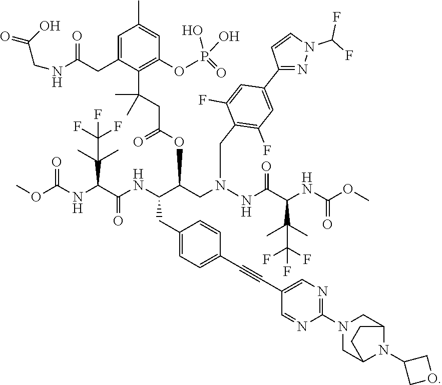 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)NCC(=O)O)cc(C)cc1OP(=O)(O)O)C(C)(C)C(F)(F)F]

160. The compound of claim 157, which is

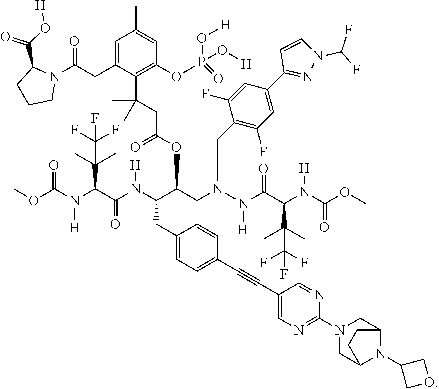 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N2CCC[C@H]2C(=O)O)cc(C)cc1OP(=O)(O)O)C(C)(C)C(F)(F)F]

161. The compound of claim 157, which is

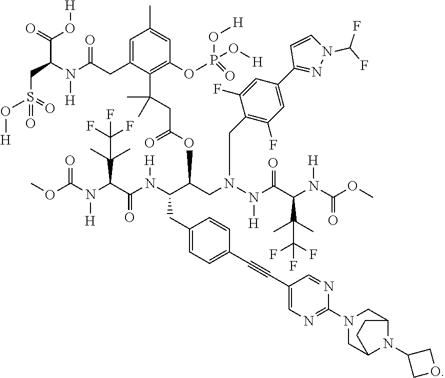 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

162. The compound of claim 157, which is

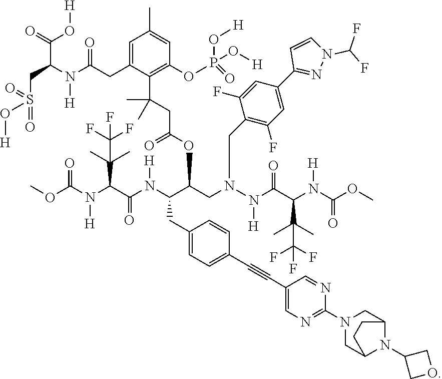 [SMILES: COC(=O)N[C@H](C(=O)N[C@@H](Cc1ccc(C#Cc2cnc(N3CC4CCC(C3)N4C3COC3)nc2)cc1)[C@H](CN(Cc1c(F)cc(-c2ccn(C(F)F)n2)cc1F)NC(=O)[C@@H](NC(=O)OC)C(C)(C)C(F)(F)F)OC(=O)CC(C)(C)c1c(CC(=O)N[C@@H](CS(=O)(=O)O)C(=O)O)cc(C)cc1OP(=O)(O)O)C(C)(C)C(F)(F)F]

163. A pharmaceutical composition comprising the compound of claim 151, or a pharmaceutically acceptable salt thereof, and a pharmaceutically acceptable excipient.

164. A pharmaceutical composition comprising the compound of claim 157, and a pharmaceutically acceptable excipient.
